(19) United States
(12) Patent Application Publication
(10) Pub. No.: US 2011/0111483 A1
(43) Pub. Date: May 12, 2011

(54) Optimizing Expression of Active Botulinum Toxin Type E

(75) Inventors: LANCE E. STEWARD, IRVINE, CA (US); MARCELLA A. GILMORE, SANTA ANA, CA (US); ESTER G. FERNANDEZ-SALAS, FULLERTON, CA (US); SHENGWEN LI, IRVINE, CA (US); RONALD G. MILLER, PUEBLO WEST, CO (US); KEI ROGER AOKI, COTO DE CAZA, CA (US)

(73) Assignee: ALLERGAN, INC., IRVINE, CA (US)

(21) Appl. No.: 12/904,983

(22) Filed: Oct. 14, 2010

Related U.S. Application Data
(63) Continuation-in-part of application No. 11/568,834, filed on Jan. 11, 2007, now Pat. No. 7,825,233.
(60) Provisional application No. 60/584,378, filed on Jun. 30, 2004.
(60) Provisional application No. 60/599,132, filed on Aug. 4, 2004.

(51) Int. Cl. C12N 1/21; C12N 15/31; C12N 15/70

(52) U.S. Cl. 435/252.33; 536/23.7; 435/320.1

(57) ABSTRACT

Nucleic acid molecules that comprise modified open reading frames providing increased expression of the encoded active BoNT/E in a heterologous cell, expression constructs and cells comprising such nucleic acid molecules and methods useful for expressing the encoding active BoNT/E from such nucleic acid molecules, expression constructs and cells.

This is a continuation-in-part application that claims priority pursuant to 35 U.S.C. §120 to U.S. Non-Provisional patent application Ser. No. 11/568,834, filed Jan. 11, 2007, a national stage application under 35 U.S.C. §371 of PCT application PCT/US2005/020578, filed Jun. 9, 2005, which claims priority pursuant to 35 U.S.C. §119(e) to U.S. Provisional Patent Application Ser. No. 60/584,378, filed Jun. 30, 2004, and provisional patent application Ser. No. 60/599,132, filed Aug. 4, 2004, each of which is all hereby incorporated by reference in its entirety.

All of the publications cited in this application are hereby incorporated by reference in their entirety. All GeneBank sequence listings cited this application, as identified by their GenBank accession numbers, are available from the National Center for Biotechnological Information and are all hereby incorporated by reference in their entirety.

The myorelaxant properties of Botulinum neurotoxins (BoNTs), such as, e.g., BoNT/A and BoNT/E, are being exploited in a wide variety of therapeutic and cosmetic applications, see e.g., William J. Lipham, COSMETIC AND CLINICAL APPLICATIONS OF BOTULINUM TOXIN (Slack, Inc., 2004). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder. In addition, BoNTs therapies, including BoNT/A and BoNT/E therapies, are proposed for treating neuromuscular disorders, see e.g., Kei Roger Aoki et al., Method for Treating Neuromuscular Disorders and Conditions with Botulinum Toxin Types A and B, U.S. Pat. No. 6,872,397 (Mar. 29, 2005); Rhett M. Schiffman, Methods for Treating Uterine Disorders, U.S. Patent Publication No. 2004/0175399 (Sep. 9, 2004); and Richard L. Barron, Methods for Treating Ulcers and Gastroesophageal Reflux Disease, U.S. Patent Publication No. 2004/0086531 (May 7, 2004); and Kei Roger Aoki, et al., Method for Treating Dystonia with Botulinum Toxin C to G, U.S. Pat. No. 6,319,505 (Nov. 20, 2001); eye disorders, see e.g., Eric R. First, Methods and Compositions for Treating Eye Disorders, U.S. Patent Publication No. 2004/0234532 (Nov. 25, 2004); Kei Roger Aoki et al., Botulinum Toxin Treatment for Blepharospasm, U.S. Patent Publication No. 2004/0151740 (Aug. 5, 2004); and Kei Roger Aoki et al., Botulinum Toxin Treatment for Strabismus, U.S. Patent Publication No. 2004/0126396 (Jul. 1, 2004); pain, see e.g., Kei Roger Aoki et al., Pain Treatment by Peripheral Administration of a Neurotoxin, U.S. Pat. No. 6,869,610 (Mar. 22, 2005); Stephen Donovan, Clostridial Toxin Derivatives and Methods to Treat Pain, U.S. Pat. No. 6,641,820 (Nov. 4, 2003); Kei Roger Aoki, et al., Method for Treating Pain by Peripheral Administration of a Neurotoxin, U.S. Pat. No. 6,464,986 (Oct. 15, 2002); Kei Roger Aoki and Minglei Cui, Methods for Treating Pain, U.S. Pat. No. 6,113,915 (Sep. 5, 2000); Martin Voet, Botulinum Toxin Therapy for Fibromyalgia, U.S. Patent Publication No. 2004/0062776 (Apr. 1, 2004); and Kei Roger Aoki et al., Botulinum Toxin Therapy for Lower Back Pain, U.S. Patent Publication No. 2004/0037852 (Feb. 26, 2004); muscle injuries, see e.g., Gregory F. Brooks, Methods for Treating Muscle Injuries, U.S. Pat. No. 6,423,319 (Jul. 23, 2002); headache, see e.g., Martin Voet, Methods for Treating Sinus Headache, U.S. Pat. No. 6,838,434 (Jan. 4, 2005); Kei Roger Aoki et al., Methods for Treating Tension Headache, U.S. Pat. No. 6,776,992 (Aug. 17, 2004); and Kei Roger Aoki et al., Method for Treating Headache, U.S. Pat. No. 6,458,365 (Oct. 1, 2002); cardiovascular diseases, see e.g., Gregory F. Brooks and Stephen Donovan, Methods for Treating Cardiovascular Diseases with Botulinum Toxin, U.S. Pat. No. 6,767,544 (Jul. 27, 2004); neurological disorders, see e.g., Stephen Donovan, Parkinson's Disease Treatment, U.S. Pat. No. 6,620,415 (Sep. 16, 2003); and Stephen Donovan, Method for Treating Parkinson's Disease with a Botulinum Toxin, U.S. Pat. No. 6,306,403 (Oct. 23, 2001); neuropsychiatric disorders, see e.g., Stephen Donovan, Botulinum toxin therapy for neuropsychiatric disorders, U.S. Patent Publication No. 2004/0180061 (Sep. 16, 2004); and Steven Donovan, Therapeutic Treatments for Neuropsychiatric Disorders, U.S. Patent Publication No. 2003/0211121 (Nov. 13, 2003); endocrine disorders, see e.g., Stephen Donovan, Method for Treating Endocrine Disorders, U.S. Pat. No. 6,827,931 (Dec. 7, 2004); Stephen Donovan, Method for Treating Thyroid Disorders with a Botulinum Toxin, U.S. Pat. No. 6,740,321 (May 25, 2004); Kei Roger Aoki et al., Method for Treating a Cholinergic Influenced Sweat Gland, U.S. Pat. No. 6,683,049 (Jan. 27, 2004); Stephen Donovan, Neurotoxin Therapy for Diabetes, U.S. Pat. No. 6,416,765 (Jul. 9, 2002); Stephen Donovan, Methods for Treating Diabetes, U.S. Pat. No. 6,337,075 (Jan. 8, 2002); Stephen Donovan, Method for Treating a Pancreatic Disorder with a Neurotoxin, U.S. Pat. No. 6,261,572 (Jul. 17, 2001); Stephen Donovan, Methods for Treating Pancreatic Disorders, U.S. Pat. No. 6,143,306 (Nov. 7, 2000); cancers, see e.g., Stephen Donovan, Methods for Treating Bone Tumors, U.S. Pat. No. 6,565,870 (May 20, 2003); Stephen Donovan, Method for Treating Cancer with a Neurotoxin to Improve Patient Function, U.S. Pat. No. 6,368,605 (Apr. 9, 2002); Stephen Donovan, Method for Treating Cancer with a Neurotoxin, U.S. Pat. No. 6,139,845 (Oct. 31, 2000); and Mitchell F. Brin and Stephen Donovan, Methods for treating diverse cancers, U.S. Patent Publication No. 2005/0031648 (Feb. 10, 2005); otic disorders, see e.g., Stephen Donovan, Neurotoxin therapy for inner ear disorders, U.S. Pat. No. 6,358,926 (Mar. 19, 2002); and Stephen Donovan, Method for Treating Otic Disorders, U.S. Pat. No. 6,265,379 (Jul. 24, 2001); as well as other disorders, see e.g., Stephen Donovan, Use of a Clostridial Toxin to Reduce Appetite, U.S. Patent Publication No. 2004/40253274 (Dec. 16, 2004); and Howard I. Katz and Andrew M. Blumenfeld, Botulinum Toxin Dental Therapies and Procedures, U.S. Patent Publication No. 2004/0115139 (Jun. 17, 2004); Kei Roger Aoki, et al., Treatment of Neuromuscular Disorders and Conditions with Different Botulinum, U.S. Patent Publication No. 2002/0010138 (Jan. 24, 2002); and Kei Roger Aoki, et al., Use of Botulinum Toxins for Treating Various Disorders and Conditions and Associated Pain, U.S. Patent Publication No. 2004/0013692 (Jan. 22, 2004). In addition, the expected use of BoNTs, such as, e.g., BoNT/A and BoNT/E, in both therapeutic and cosmetic treatments of humans is anticipated to expand to an ever widening range of diseases and aliments that can benefit from the myorelaxant properties of these toxins.

The increasing use of BoNTs therapies, such as, e.g., BoNT/A and BoNT/E, in treating a wider range of human afflictions necessitates increasing the efficiency with which these toxins are produced. However, meeting the needs for this ever increasing demand for such BoNT treatments may become difficult. One outstanding problem is that methods previously described to express BoNTs using heterologous organisms have failed to achieve optimal levels of BoNTs in commercial quantities. This inefficiency is a problem not only because the amount of BoNTs, such as, e.g., BoNT/A and BoNT/E, anticipated for future therapies is increasing, but also because this inefficiency leads to higher overall production costs. Furthermore, this difficulty is exacerbated for BoNTs that require in vitro activation by an exogenous protease, such as, e.g., BoNT/E, since the loss of toxin associated with the activation procedure require even larger amounts of starting material. Therefore, the poor yields using previously described methods is a significant obstacle to the overall commercial production of these BoNTs and is thus a major problem since active forms of these toxins are needed for scientific, therapeutic and cosmetic applications.

### BRIEF DESCRIPTION OF THE DRAWINGS

FIG. 1 shows a schematic of the current paradigm of neurotransmitter release and Clostridial toxin intoxication in a central and peripheral neuron. FIG. 1a shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1b shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including changes in the internal pH of the vesicle, formation of a channel pore comprising the H_{N} domain of Clostridial toxin heavy chain, separation of the Clostridial toxin light chain from the heavy chain, enzymatic activation of the light chain; and release of the activated light chain and 4) enzymatic target modification, where the activated light chain of Clostridial toxin proteolytically cleaves its target SNARE substrates, such as, e.g., SNAP-25, VAMP or Syntaxin, thereby preventing vesicle docking and neurotransmitter release.

FIG. 2 shows a plasmid map of prokaryotic expression construct pET28a/His-BoNT/E comprising the modified open reading frame of SEQ ID NO: 122 encoding an active BoNT/E operably-linked to an amino-terminal polyhistidine binding peptide (SEQ ID NO: 123). A Thrombin protease cleavage site is operably-linked between the polyhistidine binding peptide and BoNT/E. Abbreviations are as follows: P_{T7}, a bacteriophage T7 promoter region; 6×His, a region encoding a polyhistidine binding peptide sequence; Thrombin, a region encoding a Thrombin cleavage site; BoNT/E, a modified open reading frame encoding an active BoNT/E; T7 TT, a bacteriophage T7 transcription termination region; f1 origin, a bacteriophage f1 origin of replication; Kanamycin, a region encoding an aminophosphotransferase peptide that confers Kanamycin resistance; pBR322 ori, a pBR322 origin of plasmid replication region; lacI, a region encoding a lactose I peptide.

FIG. 3 shows a plasmid map of prokaryotic expression construct pET29a/BoNT/E-His comprising the modified open reading frame of SEQ ID NO: 124 encoding an active BoNT/E operably-linked to an carboxy-terminal polyhistidine binding peptide (SEQ ID NO: 125). A Thrombin protease cleavage site is operably-linked between the polyhistidine binding peptide and BoNT/E. Abbreviations are as follows: P_{T7}, a bacteriophage T7 promoter region; 6×His, a region encoding a polyhistidine binding peptide sequence; Thrombin, a region encoding a Thrombin cleavage site; BoNT/E, a modified open reading frame encoding an active BoNT/E; T7 TT, a bacteriophage T7 transcription termination region; f1 origin, a bacteriophage f1 origin of replication; Kanamycin, a region encoding an aminophosphotransferase peptide that confers Kanamycin resistance; pBR322 ori, a pBR322 origin of plasmid replication region; lacI, a region encoding a lactose I peptide.

FIG. 4 shows the results of a GFP-SNAP25 activity assay used to identify constructs expressing active His-BoNT/E. His-BoNT/E candidates 5, 6 and 10 showed statistically significant BoNT/E enzymatic activity.

FIG. 5 shows the results of a GFP-SNAP25 activity assay used to identify constructs expressing active BoNT/E-His. BoNT/E-His candidate 7 showed statistically significant BoNT/E enzymatic activity.

FIG. 6 shows IMAC purified BoNT/E expressed from modified open reading frames. FIG. 6a shows an IMAC purification profile of His-BoNT/E expressed from the pET28a/His-BoNT/E expression construct comprising the modified open reading frame of SEQ ID NO: 122. Amounts of His-BoNT/E obtained averaged approximately 12 mg/L and represents a four-fold increase in protein amounts obtained from an unmodified open reading frame encoding the same active BoNT/E. FIG. 6b shows an IMAC purification profile of BoNT/E-His expressed from the pET29a/BoNT/E-His expression construct comprising the modified open reading frame of SEQ ID NO: 124. Amounts of His-BoNT/E obtained averaged approximately 60 mg/L and represents a 20-fold increase in protein amounts obtained from an unmodified open reading frame encoding the same active BoNT/E.

FIG. 7 shows a plasmid map of prokaryotic expression construct pRSETb/His-BoNT/E comprising the modified open reading frame encoding an active BoNT/E operably-linked to amino-terminal polyhistidine and Express™ binding peptides. An Enterokinase protease cleavage site is operably-linked between the polyhistidine and Express™ binding peptides and BoNT/E. Abbreviations are as follows: P_{T7}, a bacteriophage T7 promoter region; 6×His, a region encoding a polyhistidine binding peptide sequence; Express™, a region encoding an Express™ binding peptide sequence; Enterokinase, a region encoding a EnterokinaseMax™ cleavage site; BoNT/E, modified open reading frame of SEQ ID NO: 7 encoding an active BoNT/E; f1 origin, a bacteriophage f1 origin of replication; Ampicillin, a region encoding a β-lactamase peptide that confers Ampicillin resistance; pBR322 ori, a pBR322 origin of plasmid replication region.

FIG. 8 shows a plasmid map of yeast expression construct pPICZ A/BoNT/E-myc-His comprising a modified open reading frame encoding an active BoNT/E operably-linked to carboxy-terminal c-myc and polyhistidine binding peptides. Abbreviations are as follows: P_{AOX1}, an aldehyde oxidase 1 promoter region; BoNT/E, modified open reading frame of SEQ ID NO: 37 encoding an active BoNT/E; c-myc, a region encoding a c-myc binding peptide sequence; 6×His, a region encoding a polyhistidine binding peptide sequence; AOX1 TT, an aldehyde oxidase 1 transcription termination region; Zeocin™, a region encoding a Zeocin™ resistance peptide; pUC ori, a pUC origin of plasmid replication region.

FIG. 9 shows a plasmid map of yeast expression construct pMET/BoNT/E-V5-His comprising a modified open reading frame encoding an active BoNT/E operably-linked to carboxy-terminal V5 and polyhistidine binding peptides. Abbreviations are as follows: P_{AUG1}, an alcohol oxidase promoter region; BoNT/E, modified open reading frame of SEQ ID NO: 37 encoding an active BoNT/E; V5, a region encoding a V5 binding peptide sequence; 6×His, a region encoding a polyhistidine binding peptide sequence; AUG1 TT, an alcohol oxidase transcription termination region; ADE2; ADE2 gene for auxotrophic selection; 3′ AUG1; pUC ori, a pUC origin of plasmid replication region; Ampicillin, a region encoding a β-lactamase peptide that confers Ampicillin resistance.

FIG. 10 shows a plasmid map of yeast expression construct pYES2.1/BoNT/E-V5-His comprising a modified open reading frame encoding an active BoNT/E operably-linked to carboxy-terminal V5 and polyhistidine binding peptides. Abbreviations are as follows: P_{GAL1}, an galactose-inducible promoter region; BoNT/E, modified open reading frame of SEQ ID NO: 40 encoding an active BoNT/E; V5, a region encoding a V5 binding peptide sequence; 6×His, a region encoding a polyhistidine binding peptide sequence; cyc1 TT, an alcohol oxidase transcription termination region; pUC ori, a pUC origin of plasmid replication region; Ampicillin, a region encoding a β-lactamase peptide that confers Ampicillin resistance; URA3; URA3 gene for auxotrophic selection; 2μ origin of replication; a 2μ origin of replication; f1 origin, a bacteriophage f1 origin of replication.

FIG. 11 shows a plasmid map of baculovirus transfer construct pFastBacHT/His-BoNT/E comprising a modified open reading frame encoding an active BoNT/E operably-linked to amino-terminal polyhistidine binding peptide. A tobacco etch virus (TEV) protease cleavage site is operably-linked between the polyhistidine binding peptide and BoNT/E. Abbreviations are as follows: P_{PH}, an polyhedrin promoter region; 6×His, a region encoding a polyhistidine binding peptide sequence; TEV, a region encoding a TEV protease cleavage sequence; BoNT/E, modified open reading frame of SEQ ID NO: 61 encoding an active BoNT/E; SV40 pA, a simian virus 40 polyadenylation site; Ampicillin, a region encoding a β-lactamase peptide that confers Ampicillin resistance; pUC ori, a pUC origin of plasmid replication region; Gentamicin, a region encoding an aminophosphotransferase peptide that confers Gentamicin resistance.

FIG. 12 shows a plasmid map of baculovirus transfer construct pBACgus3/BoNT/E-His comprising a modified open reading frame encoding an active BoNT/E operably-linked to carcoxy-terminal polyhistidine binding peptide. A thrombin protease cleavage site is operably-linked between the BoNT/E and the polyhistidine binding peptide. Abbreviations are as follows: P_{PH}, an polyhedrin promoter region; gp64, a region encoding a gp64 signal peptide; BoNT/E, modified open reading frame of SEQ ID NO: 61 encoding an active BoNT/E; Thrombin, a region encoding a Thrombin protease cleavage sequence; 6×His, a region encoding a polyhistidine binding peptide sequence; pUC ori, a pUC origin of plasmid replication region; Ampicillin, a region encoding a β-lactamase peptide that confers Ampicillin resistance; f1 origin, a bacteriophage f1 origin of replication; gus, a region encoding a β-glucuronidase peptide.

FIG. 13 shows a plasmid map of insect expression construct pMT/BiP-BoNT/E-V5-His comprising a modified open reading frame encoding an active BoNT/E operably-linked to carboxy-terminal V5 and polyhistidine binding peptides. Abbreviations are as follows: P_{MT}, an metallothionein promoter region; BipSS, a region encoding a BiP signal sequence; BoNT/E, modified open reading frame of SEQ ID NO: 58 encoding an active BoNT/E; V5, a region encoding a V5 binding peptide sequence; 6×His, a region encoding a polyhistidine binding peptide sequence; SV40 pA, a simian virus 40 polyadenylation site; pUC ori, a pUC origin of plasmid replication region; Ampicillin, a region encoding a β-lactamase peptide that confers Ampicillin resistance.

FIG. 14 shows a plasmid map of mammalian expression construct pQBI25/BoNT/E-GFP comprising a modified open reading frame encoding an active BoNT/E operably-linked to a carboxy-terminal GFP peptide. Abbreviations are as follows: P_{CMV}, an cytomegalovirus promoter region; BoNT/E, a modified open reading frame of SEQ ID NO: 97 encoding an active BoNT/E; GFP, a region encoding a Green Florescence Protein peptide; BGH pA, a bovine growth hormone polyadenylation site; Neomycin, a region encoding an aminophosphotransferase peptide that confers Neomycin resistance; pUC ori, a pUC origin of plasmid replication region; Ampicillin, a region encoding a β-lactamase peptide that confers Ampicillin resistance.

FIG. 15 shows a plasmid map of mammalian expression construct pcDNA™6/BoNT/E-V5-His comprising a modified open reading frame encoding an active BoNT/E operably-linked to carboxy-terminal V5 and polyhistidine binding peptides. Abbreviations are as follows: P_{CMV}, an cytomegalovirus promoter region; BoNT/E, a modified open reading frame of SEQ ID NO: 97 encoding an active BoNT/E; V5, a region encoding a V5 binding peptide sequence; 6×His, a region encoding a polyhistidine binding peptide sequence; BGH pA, a bovine growth hormone polyadenylation site; Blasticidin, a region encoding an blasticidin resistance peptide; pUC ori, a pUC origin of plasmid replication region; Ampicillin, a region encoding a β-lactamase peptide that confers Ampicillin resistance.

FIG. 16 shows a plasmid map of cell-free expression construct pIVEX2.3d/BoNT/E-His comprising a modified open reading frame encoding an active BoNT/E operably-linked to a carboxy-terminal polyhistidine binding peptide. Abbreviations are as follows: P_{T7}, a bacteriophage T7 promoter region; RBS, a ribosomal binding site region; BoNT/E, a modified open reading frame of SEQ ID NO: 4 encoding an active BoNT/E; 6×His, a region encoding a polyhistidine binding peptide sequence; T7 TT, a bacteriophage T7 transcription termination region; pUC ori, a pUC origin of plasmid replication region; Ampicillin, a region encoding a β-lactamase peptide that confers Ampicillin resistance.

### DETAILED DESCRIPTION

The present invention recognizes the need for the high-level, commercial production of active clostridial toxins using heterologous organisms. All clostridial toxins useful for scientific, therapeutic and cosmetic applications are envisioned including, without limitation, BoNTs, such as, e.g., BoNT/A, BoNT/B, BoNT/C, BoNT/D, BoNT/E, BoNT/F and BoNT/G, and TeNT. Furthermore, BoNTs that require in vitro activation, such as e.g., BoNT/E and BoNT/G, can also benefit from the present invention. High-level production of a clostridial toxin is achieved by using modified nucleic acid molecules which allows for increased expression of the encoded toxin in a heterologous cell and thus higher protein yields. In aspects of the present invention, nucleic acid molecules encoding a clostridial toxin comprise modified open reading frames designed to 1) contain codons typically present in the open reading frames of native nucleic acid molecules found in the heterologous cell selected to express that molecule; 2) contain a G+C content that more closely matches the average G+C content of open reading frames of native nucleic acid molecules found in the heterologous cell selected to express that molecule; 3) reduce polymononucleotide regions found within the open reading frame encoding an active clostridial toxin; and/or 4) eliminate internal regulatory or structural sites found within the open reading frame encoding an active clostridial toxin. Because a large number of production factors can influence the selection of a specific heterologous cell, nucleic acid molecules disclosed in the present specification are directed toward a wide range of prokaryotic and eukaryotic cell including, without limitation, bacteria strains, yeast strains, plant cells and cell lines derived from plants, insect cells and cell lines derived from insects and mammalian cells and cell lines derived from mammals. Aspects of the present invention also provide for expression constructs and cell compositions useful for expressing modified nucleic acid molecules disclosed in the present specification. In addition, aspects of the present invention provide methods for producing Clostridial toxins using the disclosed nucleic acid molecules.

Aspects of the present invention provide nucleic acid molecules comprising a modified open reading frame providing increased expression of the encoded active BoNT/E. The modified open reading frame includes at least one nucleotide change as compared to the unmodified open reading frame encoding the same active BoNT/E. Increased active BoNT/E expression from a modified open reading frame in a heterologous cell is determined by comparing the expression level from an unmodified open reading frame encoding the same active BoNT/E from an otherwise identical nucleic acid molecule in the same type of heterologous cell. A nucleotide change may alter a synonymous codon within the open reading frame in order to agree with the endogenous codon usage found in the heterologous cell selected to express the molecule disclosed in the present specification. Additionally, a nucleotide change may alter the G+C content within the open reading frame to better match the average G+C content of open reading frames found in endogenous nucleic acid molecules present in the heterologous cell. A nucleotide change may also alter a polymononucleotide region or an internal regulatory or structural site found within the native nucleic acid molecule. A wide variety of modified nucleic acid molecules are envisioned including, without limitation, molecules comprising a modified open reading frame providing increased expression of the encoded active BoNT/E in a prokaryotic cell; molecules comprising a modified open reading frame providing increased expression of the encoded active BoNT/E in a yeast cell; molecules comprising a modified open reading frame providing increased expression of the encoded active BoNT/E in an insect cell; and molecules comprising a modified open reading frame providing increased expression of the encoded active BoNT/E in a mammalian cell.

Other aspects of the present invention provide expression constructs comprising a nucleic acid molecule disclosed in the present specification, operably-linked to an expression vector useful for expressing the nucleic acid molecule in a heterologous cell. A wide variety of expression vectors are envisioned, including, without limitation, a prokaryotic expression vector useful for expressing a nucleic acid molecule comprising a modified open reading frame providing increased expression of the encoded active BoNT/E in a prokaryotic cell; a yeast expression vector useful for expressing a nucleic acid molecule comprising a modified open reading frame providing increased expression of the encoded active BoNT/E in a yeast cell; an insect expression vector useful for expressing a nucleic acid molecule comprising a modified open reading frame providing increased expression of the encoded active BoNT/E in an insect cell; a mammalian expression vector useful for expressing a nucleic acid molecule comprising a modified open reading frame providing increased expression of the encoded active BoNT/E in a mammalian cell.

Aspects of the present invention further provide heterologous cells comprising an expression construct disclosed in the present specification. It is envisioned that a cell can include, without limitation, a prokaryotic cell containing a prokaryotic expression construct useful for expressing a nucleic acid molecule comprising a modified open reading frame providing increased expression of the encoded active BoNT/E in a prokaryotic cell; a yeast cell containing a yeast expression construct useful for expressing a nucleic acid molecule comprising a modified open reading frame providing increased expression of the encoded active BoNT/E in a yeast cell; an insect cell containing an insect expression construct useful for expressing a nucleic acid molecule comprising a modified open reading frame providing increased expression of the encoded active BoNT/E in an insect cell; and a mammalian cell containing a mammalian expression construct useful for expressing a nucleic acid molecule comprising a modified open reading frame providing increased expression of the encoded active BoNT/E in a mammalian cell.

Other aspects of the present invention provide methods of producing an active BoNT/E comprising the step of expressing a nucleic acid molecule in a heterologous cell, the nucleic acid molecule comprising a modified open reading frame encoding the active BoNT/E. Aspects of these methods use nucleic acid molecules, expression constructs and cells disclosed in the present specification. It is envisioned that both cell-free and cell-based expression systems can be used to produce an active BoNT/E disclosed in the present specification according to this method.

Aspects of the present invention provide, in part, nucleic acid molecules comprising a modified open reading frame encoding active BoNT/E in a heterologous cell. As used herein, the term “open reading frame” is synonymous with “ORF” and means any nucleotide sequence that is potentially able to encode a protein, or a portion of a protein. An open reading frame usually begins with a start codon (represented as, e.g. AUG for an RNA molecule and ATG in a DNA molecule in the standard code) and is read in codon-triplets until the frame ends with a STOP codon (represented as, e.g. UAA, UGA or UAG for an RNA molecule and TAA, TGA or TAG in a DNA molecule in the standard code). As used herein, the term “codon” means a sequence of three nucleotides in a nucleic acid molecule that specifies a particular amino acid during protein synthesis; also called a triplet or codon-triplet. For example, of the 64 possible codons in the standard genetic code, two codons, GAA and GAG encode the amino acid Glutamine whereas the codons AAA and AAG specify the amino acid Lysine. In the standard genetic code three codons are stop codons, which do not specify an amino acid. As used herein, the term “synonymous codon” means any and all of the codons that code for a single amino acid. Except for Methionine (Met) and Tryptophan (Trp), amino acids are coded by two to six synonymous codons (see e.g., Table 1). For example, in the standard genetic code the four synonymous codons that code for the amino acid Alanine are GCA, GCC, GCG and GCU, the two synonymous codons that specify Glutamine are GAA and GAG and the two synonymous codons that encode Lysine are AAA and AAG (for other non-limiting examples see Table 1).

Thus in an embodiment, a modified open reading frame that encodes an active BoNT/E is changed by altering the nucleotide sequence of native Clostridia botulinum codons to better match the synonymous codons used by the heterologous cell selected to express nucleic acid molecules disclosed in the present specification. The C. botulinum strain that expresses BoNT/E exhibits a specific preference or bias for one synonymous codon over the others and there is a direct correlation between this C. botulinum strain-specific codon usage and the cellular concentration of the corresponding isoacceptor tRNA. This unequal presence of synonymous codons in a known or predicted open reading frame in an organism-, cell-, or functional class-specific manner is a phenomenon called codon bias or codon preference. Thus, it can be said that a heterologous cell has a bias for one synonymous codon over another synonymous codon, or that a heterologous cell prefers one synonymous codon over another synonymous codon. In addition, the synonymous codon to which the most abundant isoacceptor tRNA equates is often different between organisms, and, in some cases, between cells comprising different tissue types of the same organism, or between functional classes of proteins of the same organism, e.g., proteins expressed during exponential growth phase of a bacterium relative to proteins expressed during stationary growth phase of a bacterium. Different codon bias may also occur through the length of the open reading frame, such as, e.g., codons from the 5′ third of the open reading frame may use different codons relative the remainind 3′ two-thirds of the same open reading frame. For example, as mentioned above, GCA, GCC, GCG and GCU are the four synonymous codons that encode Alanine (Ala). While the most abundant Ala isoacceptor representative in C. botulinum recognizes the GCA codon, the bacterium Escherichia coli recognizes GCG, the yeast Pichia pastoris recognizes GCT and most multicellular eukaryotes appear to recognizes GCC (see e.g., Table 1). Thus, certain codons that are normally used in the Clostridia botulinum strain that expresses BoNT/E may be rarely present in heterologous cells commonly used in the commercial expression of BoNT/E. Because these heterologous organisms do not produce the corresponding isoacceptor tRNAs at a concentration sufficient to support high-level BoNT/E expression, optimal protein yields are not achieved. Therefore, a modified open reading frame comprising nucleotide changes that increase the number of synonymous codons preferred by a heterologous cell will provide increased expression of the encoded active BoNT/E as compared to an unmodified open reading frame encoding the same active BoNT/E. A synonymous codon of the open reading frame can be changed by substituting a nucleotide at the third position of a codon with a different nucleotide, while still retaining the identity of the amino acid coded by that codon. As a non-limiting example, a 5′-AAATACTTA-3′ (SEQ ID NO: 126) open reading frame encoding the tripeptide NH2-lysine-tyrosine-leucine-COOH can be changed to 5′-AAGTATCTG-3′ (SEQ ID NO: 127) and still encode the tripeptide NH2-lysine-tyrosine-leucine-COOH.

Thus, in an aspect of this embodiment, at least one nucleotide change is made to a nucleic acid molecule that substitutes a codon in the open reading frame for a synonymous codon providing increased expression of the encoded active BoNT/E in a heterologous cell. In another aspect of this embodiment, a plurality of nucleotide changes are made to a nucleic acid molecule that substitutes a plurality of codons in the open reading frame for a plurality of synonymous codon providing increased expression of the encoded active BoNT/E in a heterologous cell. Thus, aspects of this embodiment can include a modified open reading frame comprises nucleotide changes that alter, e.g., at least 10 synonymous codons, at least 25 synonymous codons, at least 50 synonymous codons, at least 75 synonymous codons, at least 100 synonymous codons, at least 200 synonymous codons, at least 300 synonymous codons, at least 400 synonymous codons, at least 500 synonymous codons, at least 600 synonymous codons, at least 700 synonymous codons, at least 800 synonymous codons, at least 900 synonymous codons, at least 1000 synonymous codons, at least 1100 synonymous codons or at least 1200 synonymous codons. In other aspects of this embodiment a modified open reading frame comprises nucleotide changes that alter, e.g., at most 10 synonymous codons, at most 25 synonymous codons, at most 50 synonymous codons, at most 75 synonymous codons, at most 100 synonymous codons, at most 200 synonymous codons, at most 300 synonymous codons, at most 400 synonymous codons, at most 500 synonymous codons, at most 600 synonymous codons, at most 700 synonymous codons, at most 800 synonymous codons, at most 900 synonymous codons, at most 1000 synonymous codons, at most 1100 synonymous codons or at most 1200 synonymous codons.

In another embodiment, a modified open reading frame encoding an active BoNT/E is changed by altering the native Clostridial botulinum G+C content to better match the G+C content found in the heterologous cell selected to express nucleic acid molecules disclosed in the present specification. The average guanine and cytosine content (referred to as the G+C content) of the C. botulinum nucleic acid molecule comprising the open reading frame encoding BoNT/E is approximately 25%. This very low G+C content is in contrast to the approximately 50% G+C content of endogenous nucleic acid molecules encoding proteins found in heterologous cells commonly used in the commercial expression of BoNT/E (see e.g. Table 2). This unequal G+C content in a known or predicted open reading frame in an organism-specific manner is a phenomenon called G+C content bias or G+C content preference. Thus, it can be said that a heterologous cell has a bias for a certain G+C content level as compared to a different G+C content level, or that a heterologous cell prefers a certain G+C content level as compared to a different G+C content. The low G+C content of the open reading frame encoding BoNT/E conversely results in higher regions of adenine and thymidine content (A+T content). Higher A+T content appears to disrupt protein expression in a heterologous cell because these regions may, for example, mimic regulatory signals that could terminate transcriptional or translational expression, form secondary structures that could hinder transcriptional or translational read-through, or comprise repetitive sequences that could promote transcriptional or translational slippage. Thus, the average G+C content of the open reading frame can influence the expression levels of BoNT/E in a heterologous cell. Therefore, a modified open reading frame comprising nucleotide changes that increase the total G+C content to a level preferred by a heterologous cell will provide increased expression of the encoded active BoNT/E as compared to an unmodified open reading frame encoding the same active BoNT/E. The G+C content of the sequence can be increased by substituting an adenine or thymidine at the third position of a codon with a guanine or cytosine, while still retaining the same amino acid coded by that codon. As a non-limiting example, a 5′-AAATATTTA-3′ (SEQ ID NO: 128) region in frame with the open reading frame could be changed to 5′-AAGTACCTG-3′ (SEQ ID NO: 129) and still code for the tripeptide NH2-lysine-tyrosine-leucine-COOH. Conversely, the G+C content of the sequence can be decreased by substituting a guanine or cytosine at the third position of a codon with an adenine or thymidine, while still retaining the same amino acid coded by that codon. As a non-limiting example, a 5′-AAGTACCTG-3′ (SEQ ID NO: 129) open reading frame encoding NH2-lysine-tyrosine-leucine-COOH can be changed to 5′-AAATATTTA-3′ (SEQ ID NO: 128) and still encode the tripeptide NH2-lysine-tyrosine-leucine-COOH.

Thus in an aspect of this embodiment, at least one nucleotide change is made to a nucleic acid molecule that alters the G+C content of an open reading frame providing increased expression of the encoded active BoNT/E in a heterologous cell. In another aspect of this embodiment, a plurality of nucleotide substitutions are made to a nucleic acid molecule that alters the G+C content of an open reading frame providing increased expression of the encoded active BoNT/E in a heterologous cell. Therefore, aspects of this embodiment include a modified open reading frame comprising nucleotide changes that increase the total G+C content level to, e.g., at least 30% total G+C content, at least 40% total G+C content, at least 50% total G+C content, at least 60% total G+C content or at least 70% total G+C content. Other aspects of this embodiment include a modified open reading frame comprising nucleotide changes that increase the total G+C content level to, e.g., at most 30% total G+C content, at most 40% total G+C content, at most 50% total G+C content, at most 60% total G+C content or at most 70% total G+C content. Furthermore, such an open reading frame can include altering the total G+C content to any 50 consecutive nucleotides by, e.g., at least 30% total G+C content, at least 40% total G+C content, at least 50% total G+C content, at least 60% total G+C content or at least 25% total G+C content. In other aspects, a modified open reading frame can include altering the total G+C content to any 75 consecutive nucleotides by, e.g., at least 30% total G+C content, at least 40% total G+C content, at least 50% total G+C content, at least 60% total G+C content or at least 25% total G+C content. In yet other aspects, a modified open reading frame can include altering the total G+C content to any 100 consecutive nucleotides by, e.g., at least 30% total G+C content, at least 40% total G+C content, at least 50% total G+C content, at least 60% total G+C content or at least 25% total G+C content.

In another embodiment, a modified open reading frame encoding an active BoNT/E is changed by altering a polymononucleotide region. Polymononucleotide regions (i.e., polyadenine, polyA; polythymidine, polyT; polyguanine, polyG; and polycytosine, polyC) can be detrimental to protein synthesis, especially if these regions are composed of five or more nucleotides. These regions can, for example, 1) contribute to translational staling which reduces the rate of protein synthesis as well as increase the numbers of incomplete/partial peptides synthesized; and 2) participate in translational skipping where the translational apparatus becomes misaligned with the open reading frame thereby producing aberrant proteins that are, e.g., truncated or contain a different amino acid sequence due to a frame shift. A polymononucleotide region can be changed by substituting a nucleotide different from the one contained in the polymononucleotide region at the third position of a codon that interrupts the region while still maintaining the same amino acid coded by the codon. As a non-limiting example, a polyA region containing nine adenosines (i.e., 5′-AAAAAAAAA-3′; SEQ ID NO: 130) encoding the tripeptide NH2-lysine-lysine-lysine-COOH can be eliminated by changing the sequence to 5′-AAGAAGAAG-3′ (SEQ ID NO: 131) and still encode the tripeptide NH2-lysine-lysine-lysine-COOH.

Thus in an aspect of this embodiment, at least one nucleotide change may be made to a nucleic acid molecule that alters a polymononucleotide region found in an open reading frame providing increased expression of the encoded active BoNT/E. In another aspect of this embodiment, a plurality of nucleotide changes are made to a nucleic acid molecule that alter a plurality of polymononucleotide regions in an open reading frame providing increased expression of the encoded active BoNT/E. In aspects of this embodiment an open reading frame can include, e.g., at least one nucleotide change, at least two nucleotide changes, at least three nucleotide changes, at least four nucleotide changes, at least five nucleotide changes, at least 10 nucleotide, at least 20 nucleotide, or at least 30 nucleotide changes.

In other aspects of this embodiment an open reading frame can include, e.g., at most one nucleotide change, at most two nucleotide changes, at most three nucleotide changes, at most four nucleotide changes, at most five nucleotide changes, at most 10 nucleotide changes, at most 20 nucleotide changes, or at most 30 nucleotide changes.

In another embodiment, a modified open reading frame is changed by altering the nucleotide sequence that alters an internal regulatory or structural site. Internal regulatory or structural sites, include, without limitation, internal or cryptic translational start sites, RNase cleavage sites, out-of-frame stop codons, methylation sites and hairpin-loop structures Internal translational start sites can misdirected the translational apparatus to an incorrect start site, thereby increasing the number of incomplete/partial or abnormal proteins synthesized. The presence of out-of-frame stop codons in the second and third reading frames of an open reading frame can increase translational efficiency and thus protein yields. For example, if the translational apparatus shifts to a reading frame not encoding the desired protein, time, resources and energy will be wasted translating defective proteins. The presence of out-of-frame stop codons reduces the cellular efforts expended in translating these aberrant peptides. RNases are enzymes that cleave RNA molecules, thereby destroying transcripts encoding a protein of interest and reducing yields. Hairpin-loop structures can physically block or disrupt the translational apparatus, thereby preventing protein synthesis or increasing the number of incomplete/partial or abnormal peptides synthesized. An internal regulatory or structural site can be changed by substituting a nucleotide different from the one contained in the consensus sequence, altering the nucleotide identity to the consensus sequence while still maintaining the same amino acid coded by the codon present in the in-frame reading frame.

In an aspect of this embodiment, a modified open reading frame is changed by altering the nucleotide sequence that alters an internal translational start site. An internal translational start site can be changed by substituting a nucleotide different from the one contained in the consensus sequence at the third position of a codon, reducing the nucleotide identity to the consensus sequence while still maintaining the same amino acid coded by the codon. As a non-limiting example, the typical translational start site in the insect Drosophila melanogaster is 5′-ACAACCAAAATG-3′, (SEQ ID NO: 132) and is present within an open reading frame would encode the peptide NH2-threonine-threonine-lysine-methionine-COOH (SEQ ID NO: 133). This translational start site can be eliminated by changing the sequence to 5′-ACGACTAAGATG-3′ (SEQ ID NO: 134) and still encode the peptide NH2-threonine-threonine-lysine-methionine-COOH (SEQ ID NO: 133). In another aspect of this embodiment, at least one nucleotide change may be made to a nucleic acid molecule altering the consensus sequence of an internal translational start site found in an open reading frame providing increased expression of the encoded active BoNT/E. In another aspect of this embodiment, a plurality of nucleotide changes are made to a nucleic acid molecule altering one or more internal translational start sites of an open reading frame providing increased expression of the encoded active BoNT/E. Therefore, aspects of this embodiment an open reading frame can include, e.g., at least one nucleotide change, at least two nucleotide changes, at least three nucleotide changes, at least four nucleotide changes, at least five nucleotide changes or at least 10 nucleotide. In other aspects of this embodiment an open reading frame can include, e.g., at most one nucleotide change, at most two nucleotide changes, at most three nucleotide changes, at most four nucleotide changes, at most five nucleotide changes, or at most 10 nucleotide changes.

In another aspect of this embodiment, a modified open reading frame is changed by altering the nucleotide sequence that alters a RNase cleavage site. A RNase cleavage site can be changed by substituting a nucleotide different from the one contained in the consensus sequence at the third position of a codon, reducing the nucleotide identity to the consensus sequence while still maintaining the same amino acid coded by the codon. As a non-limiting example, the typical RNase E cleavage site is 5′-GGTAATTGC-3′ (SEQ ID NO: 135) is present within an open reading frame and encodes the peptide NH2-glycine-isoleucine-cysteine-COOH. This RNase cleavage site can be eliminated by changing the sequence to 5′-GGCAACTGC-3′ (SEQ ID NO: 136) and still encode the peptide NH₂-threonine-threonine-lysine-methionine-COOH. In another aspect of this embodiment, at least one nucleotide change may be made to a nucleic acid molecule altering the consensus sequence of a RNase cleavage site found in an open reading frame providing increased expression of the encoded active BoNT/E. In another aspect of this embodiment, a plurality of nucleotide changes are made to a nucleic acid molecule altering one or more RNase cleavage sites of an open reading frame providing increased expression of the encoded active BoNT/E. Therefore, aspects of this embodiment an open reading frame can include, e.g., at least one nucleotide change, at least two nucleotide changes, at least three nucleotide changes, at least four nucleotide changes, at least five nucleotide changes or at least 10 nucleotide. In other aspects of this embodiment an open reading frame can include, e.g., at most one nucleotide change, at most two nucleotide changes, at most three nucleotide changes, at most four nucleotide changes, at most five nucleotide changes, or at most 10 nucleotide changes.

In another aspect of this embodiment, a modified open reading frame is changed by altering the nucleotide sequence to add a stop codon to an out-of-frame reading frame. A stop codon in an out-of-frame reading frame can be added by substituting a nucleotide different from the one contained in the consensus sequence of the stop codon, reducing the nucleotide identity to the consensus sequence while still maintaining the same amino acid coded by the in-frame codon. As a non-limiting example, the in-frame open reading frame of the nucleotide sequence 5′-GGCAACTGC-3′ (SEQ ID NO: 137) encodes the peptide NH2-glycine-isoleucine-cysteine-COOH. An out of frame stop codon can be added by changing the sequence to 5′-GGTAACTGC-3′ (SEQ ID NO: 138) (underlined sequence) and still encode the peptide NH₂-glycine-isoleucine-cysteine-COOH. In another aspect of this embodiment, at least one nucleotide change may be made to a nucleic acid molecule adding a stop codon to an out-of-frame reading frame providing increased expression of the encoded active BoNT/E. In another aspect of this embodiment, a plurality of nucleotide changes are made to a nucleic acid molecule adding one or more stop codons to an out-of-frame reading frame providing increased expression of the encoded active BoNT/E. Therefore, aspects of this embodiment an out of frame reading frame can include, e.g., at least one nucleotide change, at least two nucleotide changes, at least three nucleotide changes, at least four nucleotide changes, at least five nucleotide changes, at least 10 nucleotide, at least 20 nucleotide, or at least 30 nucleotide changes. In other aspects of this embodiment an out of frame reading frame can include, e.g., at most one nucleotide change, at most two nucleotide changes, at most three nucleotide changes, at most four nucleotide changes, at most five nucleotide changes, at most 10 nucleotide changes, at most 20 nucleotide changes, or at most 30 nucleotide changes.

In another aspect of this embodiment, a modified open reading frame is changed by altering the nucleotide sequence that alters a hairpin-loop structure. A hairpin-loop structure can be changed by substituting a nucleotide different from the one contained in the consensus sequence at the third position of a codon, reducing the nucleotide identity to the consensus sequence while still maintaining the same amino acid coded by the codon. As a non-limiting example, the hairpin-loop structure 5′-GCTTGGCCAAGC-3′ (SEQ ID NO: 139) is present within an open reading frame and encodes the peptide NH2-alanine-tryptophan-proline-serine-COOH. This hairpin-loop structure can be eliminated by changing the sequence to 5′-GCATGGCCTAGC-3′ (SEQ ID NO: 140) and still encode the peptide NH₂-alanine-tryptophan-proline-serine-COOH. In another aspect of this embodiment, at least one nucleotide change may be made to a nucleic acid molecule altering the consensus sequence of a hairpin-loop structure found in an open reading frame providing increased expression of the encoded active BoNT/E. In another aspect of this embodiment, a plurality of nucleotide changes are made to a nucleic acid molecule altering the consensus sequence of a hairpin-loop structure found in an open reading frame providing increased expression of the encoded active BoNT/E. Therefore, aspects of this embodiment an open reading frame can include, e.g., at least one nucleotide change, at least two nucleotide changes, at least three nucleotide changes, at least four nucleotide changes, at least five nucleotide changes, at least 10 nucleotide, at least 20 nucleotide, or at least 30 nucleotide changes. In other aspects of this embodiment an open reading frame can include, e.g., at most one nucleotide change, at most two nucleotide changes, at most three nucleotide changes, at most four nucleotide changes, at most five nucleotide changes, at most 10 nucleotide changes, at most 20 nucleotide changes, or at most 30 nucleotide changes.

In yet another embodiment, a modified open reading frame is changed, as compared to the open reading frame of SEQ ID NO: 3, altering synonymous codons, G+C content, polymononucleotide regions and internal regulatory or structural sites, or any combination thereof, providing increased expression of the encoded active BoNT/E.

In an aspect of this embodiment, at least one nucleotide change is made to a nucleic acid molecule that substitutes a codon in the open reading frame for a synonymous codon and alters the G+C content of an open reading frame providing increased expression of the encoded active BoNT/E. In another aspect of this embodiment, a plurality of nucleotide changes are made to a nucleic acid molecule that substitutes a plurality of codons in the open reading frame for a plurality of synonymous codon and alters the G+C content of an open reading frame providing increased expression of the encoded active BoNT/E. In another aspect of this embodiment, at least one nucleotide change is made to a nucleic acid molecule that substitutes a codon in the open reading frame for a synonymous codon and alters a polymononucleotide region found in an open reading frame providing increased expression of the encoded active BoNT/E. In another aspect of this embodiment, a plurality of nucleotide changes are made to a nucleic acid molecule that substitutes a plurality of codons in the open reading frame for a plurality of synonymous codon and alters a plurality of polymononucleotide region found in an open reading frame providing increased expression of the encoded active BoNT/E. In a further aspect of this embodiment, at least one nucleotide change is made to a nucleic acid molecule that substitutes a codon in the open reading frame for a synonymous codon and alters an internal regulatory or structural site found in an open reading frame providing increased expression of the encoded active BoNT/E. In another aspect of this embodiment, a plurality of nucleotide changes are made to a nucleic acid molecule that substitutes a plurality of codons in the open reading frame for a plurality of synonymous codon and alters a plurality of internal regulatory or structural sites found in an open reading frame providing increased expression of the encoded active BoNT/E.

In still another aspect of this embodiment, at least one nucleotide change is made to a nucleic acid molecule that substitutes a codon in the open reading frame for a synonymous codon, alters the G+C content of an open reading frame and alters a polymononucleotide region providing increased expression of the encoded active BoNT/E. In another aspect of this embodiment, a plurality of nucleotide changes are made to a nucleic acid molecule that substitutes a plurality of codons in the open reading frame for a plurality of synonymous codon, alters the G+C content of an open reading frame and alters a plurality of polymononucleotide region providing increased expression of the encoded active BoNT/E. In yet another aspect of this embodiment, at least one nucleotide change is made to a nucleic acid molecule that substitutes a codon in the open reading frame for a synonymous codon, alters the G+C content of an open reading frame and alters an internal regulatory or structural site providing increased expression of the encoded active BoNT/E. In another aspect of this embodiment, a plurality of nucleotide changes are made to a nucleic acid molecule that substitutes a plurality of codons in the open reading frame for a plurality of synonymous codon, alters the G+C content of an open reading frame and alters a plurality of internal regulatory or structural sites providing increased expression of the encoded active BoNT/E.

In an aspect of this embodiment, at least one nucleotide change is made to a nucleic acid molecule that substitutes a codon in the open reading frame for a synonymous codon, alters the G+C content of an open reading frame, alters a polymononucleotide region and alters an internal regulatory or structural site providing increased expression of the encoded active BoNT/E. In another aspect of this embodiment, a plurality of nucleotide changes are made to a nucleic acid molecule that substitutes a plurality of codons in the open reading frame for a plurality of synonymous codon, alters the G+C content of an open reading frame, alters a plurality of polymononucleotide region and alters a plurality of internal regulatory or structural sites providing increased expression of the encoded active BoNT/E.

Non-limiting examples of nucleic acid molecules disclosed in the present specification include the nucleic acid sequence molecules comprising SEQ ID NO: 4 through SEQ ID NO: 97, SEQ ID NO: 117, SEQ ID NO: 122 and SEQ ID NO: 124.

In aspects of this embodiment, a nucleic acid sequence molecule has, e.g., about 70%, about 75%, about 80%, about 85%, about 90%, about 95%, or about 97% nucleic acid sequence identity to SEQ ID NO: 4, SEQ ID NO: 5, SEQ ID NO: 6 SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33 or SEQ ID NO: 34, SEQ ID NO: 35, SEQ ID NO: 36, SEQ ID NO: 37 SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 or SEQ ID NO: 46, SEQ ID NO: 56, SEQ ID NO: 57, SEQ ID NO: 58 SEQ ID NO: 59, SEQ ID NO: 60 or SEQ ID NO: 61, SEQ ID NO: 74, SEQ ID NO: 75, SEQ ID NO: 76 SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88 SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 or SEQ ID NO: 97, SEQ ID NO: 117, SEQ ID NO: 122, or SEQ ID NO: 124.

In aspects of this embodiment, a nucleic acid sequence molecule has, e.g., at least 70%, at least 75%, at least 80%, at least 85%, at least 90%, at least 95%, or at least 97% nucleic acid sequence identity to SEQ ID NO: 4, SEQ ID NO: 5, SEQ ID NO: 6 SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33 or SEQ ID NO: 34, SEQ ID NO: 35, SEQ ID NO: 36, SEQ ID NO: 37 SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 or SEQ ID NO: 46, SEQ ID NO: 56, SEQ ID NO: 57, SEQ ID NO: 58 SEQ ID NO: 59, SEQ ID NO: 60 or SEQ ID NO: 61, SEQ ID NO: 74, SEQ ID NO: 75, SEQ ID NO: 76 SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88 SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 or SEQ ID NO: 97, SEQ ID NO: 117, SEQ ID NO: 122, or SEQ ID NO: 124.

In aspects of this embodiment, a nucleic acid sequence molecule has, e.g., about 85% to about 97%, about 90% to about 97%, about 95% to about 97%, about 85% to about 99%, about 90% to about 99%, about 95% to about 99%, or about 97% to about 99%, about 85% to about 100%, about 90% to about 100%, about 95% to about 100%, or about 97% to about 100% nucleic acid sequence identity to SEQ ID NO: 4, SEQ ID NO: 5, SEQ ID NO: 6 SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33 or SEQ ID NO: 34, SEQ ID NO: 35, SEQ ID NO: 36, SEQ ID NO: 37 SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 or SEQ ID NO: 46, SEQ ID NO: 56, SEQ ID NO: 57, SEQ ID NO: 58 SEQ ID NO: 59, SEQ ID NO: 60 or SEQ ID NO: 61, SEQ ID NO: 74, SEQ ID NO: 75, SEQ ID NO: 76 SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88 SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96 or SEQ ID NO: 97, SEQ ID NO: 117, SEQ ID NO: 122, or SEQ ID NO: 124.

It is envisioned that any of a variety of additional nucleotide modifications can be done to assist in the making and using of a nucleic acid molecule and the active BoNT/E encoded by such molecules. In one embodiment, a nucleic acid molecule disclosed in the present specification can be modified to add at least one nucleotide sequence region comprising a restriction endonuclease binding site. In another aspect of this embodiment, a molecule disclosed in the present specification can include a plurality of restriction endonuclease binding sites. Therefore, aspects of this embodiment can include a nucleic acid molecule that includes a nucleic acid region comprising one or more restriction endonuclease binding sites, two or more restriction endonuclease sites, three or more restriction endonuclease sites, four or more restriction endonuclease sites, or five or more restriction endonuclease enzyme sites. It is envisioned that the location of a nucleic acid region comprising a restriction endonuclease binding site can be at the 5′ end of a molecule, the 3′ end of the molecule, within the molecule, or any combination thereof. In another aspect of this embodiment, regions comprising restriction endonuclease sites are added to both the 5′ and 3′ ends of the open reading frame contained in a nucleic acid molecule. In another aspect of this embodiment, restriction endonuclease sites flank each end of an open reading frame encoding the BoNT/E of SEQ ID NO: 1. Any of a wide variety of restriction endonuclease binding sites can be used with nucleic acid molecules disclosed in the present specification. The selection, making and use of restriction endonuclease binding sites are routine procedures well within the scope of one skilled in the art and from the teaching herein.

In another embodiment, nucleic acid molecules disclosed in the present specification can include at least one nucleotide change that eliminates a restriction endonuclease binding site from within an open reading frame. In another aspect of this embodiment, a molecule disclosed in the present specification can include a plurality of nucleotide substitutions that eliminate a restriction endonuclease binding site from within an open reading frame. Therefore, aspects of this embodiment can include a nucleic acid molecule that alters the recognition sequence of a restriction endonuclease binding site found within an open reading frame by one or more nucleotides, two or more nucleotides, three or more nucleotides, or four or more nucleotides. A restriction endonuclease binding site can be altered by substituting a nucleotide different from the one contained in the palindrome recognition sequence of that enzyme at the third position of a codon that interrupted the site while still maintaining the same amino acid coded by the codon. As a non-limiting example, an EcoRI recognition site of 5′-GAATTC-3′, found in the open reading frame, encoding for the dipeptide NH₂-glutamate-phenylalanine-COOH can be changed to 5′-GAGTTC-3′ to eliminate the EcoRI recognition site and still code for the dipeptide NH₂-glutamate-phenylalanine-COOH. In yet another aspect of this embodiment, a nucleic acid molecule disclosed in the present specification can include the elimination of at least one restriction endonuclease site from an open reading frame. In yet another aspect of this embodiment, a molecule disclosed in the present specification can include the elimination of a plurality of restriction endonuclease binding sites from an open reading frame. Thus, aspects of this embodiment can eliminate one or more restriction endonuclease binding sites from an open reading frame, two or more restriction endonucleases binding site from an open reading frame, three or more restriction endonucleases binding site from an open reading frame, or four or more restriction endonucleases binding site from an open reading frame.

In yet another embodiment, nucleic acid molecules disclosed in the present specification can include at least one nucleic acid region encoding a binding peptide. Such a binding peptide is operably-linked in-frame to an open reading frame encoding a BoNT/E as a fusion protein. In another aspect of this embodiment, a nucleic acid molecule disclosed in the present specification can include a plurality of nucleic acid regions encoding multiple operably-linked binding peptides. Therefore, aspects of this embodiment can include a nucleic acid molecule including a one nucleic acid region encoding one or more operably-linked binding peptides, two or more operably-linked binding peptides, three or more operably-linked binding peptides, four or more operably-linked binding peptides, or five or more operably-linked binding peptides. In another aspect of this embodiment, nucleic acid regions comprising multiple binding peptides can encode multiple copies of the same binding peptide, different binding peptides, or any combination thereof. The location of a nucleic acid region encoding a binding peptide may be in various positions, including, without limitation, before the amino terminus of the BoNT/E, within the BoNT/E, or after the carboxy terminus of the BoNT/E and a binding peptide. Examples of binding peptides that can be encoded by a nucleic acid region disclosed in the present specification include, without limitation, epitope-binding peptides such as FLAG, Express™, human Influenza virus hemagluttinin (HA), human p62^{c-Myc} protein (c-MYC), Vesicular Stomatitis Virus Glycoprotein (VSV-G), glycoprotein-D precursor of Herpes simplex virus (HSV), V5, and AU1; affinity-binding peptides such as polyhistidine (HIS), streptavidin binding peptide (strep), and biotin; and peptide-binding domains such as the glutathione binding domain of glutathione-S-transferase, the calmodulin binding domain of the calmodulin binding protein, and the maltose binding domain of the maltose binding protein. Non-limiting examples of specific protocols for selecting, making and using an appropriate binding peptide are described in, e.g., MOLECULAR CLONING A LABORATORY MANUAL (Joseph Sambrook & David W. Russell eds., Cold Spring Harbor Laboratory Press, 3^{rd} ed. 2001); ANTIBODIES: A LABORATORY MANUAL (Edward Harlow & David Lane, eds., Cold Spring Harbor Laboratory Press, 2^{nd} ed. 1998); and USING ANTIBODIES: A LABORATORY MANUAL: PORTABLE PROTOCOL No. I (Edward Harlow & David Lane, Cold Spring Harbor Laboratory Press, 1998), which are hereby incorporated by reference. In addition, non-limiting examples of binding peptides as well as well-characterized reagents, conditions and protocols are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; and Stratagene, La Jolla, Calif. These protocols are routine procedures within the scope of one skilled in the art and from the teaching herein.

In yet another embodiment, a nucleic acid molecules disclosed in the present specification can include at least one nucleic acid region encoding a protease cleavage site. Such a protease cleavage site is operably-linked in-frame to an open reading frame encoding an active BoNT/E and a binding peptide as a fusion protein. In another aspect of this embodiment, a molecule disclosed in the present specification can comprise a plurality of one nucleic acid regions encoding multiple protease cleavage sites. It is further envisioned that in a molecule containing two or more one nucleic acid regions, these regions may encode the same protease cleavage sites or may encode for different protease cleavage sites. The location of the one nucleic acid region encoding the cleavage site may be in various positions, including, without limitation, between a binding peptide and the amino terminus of the active BoNT/E or between the carboxy terminus of the active BoNT/E and a binding peptide element. Examples of protease cleavage sites that can be encoded by a nucleic acid region disclosed in the present specification include, without limitation, an enterokinase cleavage site, a thrombin cleavage site, a Factor Xa cleavage site, a tobacco etch virus (TEV) protease cleavage site, a dipeptidyl aminopeptidase cleavage site and a small ubiquitin-like modifier (SUMO)/ubiquitin-like protein-1(ULP-1) protease cleavage site. Non-limiting examples of protease cleavage site as well as well-characterized reagents, conditions and protocols are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; and Stratagene, La Jolla, Calif. The selection, making and use of an appropriate protease cleavage site are routine procedures within the scope of one skilled in the art and from the teaching herein.

It is envisioned that any of a variety of means can be used to identify appropriate nucleotides to change in order to make a modified open reading frame providing increased expression of an active BoNT/E. Appropriate nucleotide changes can be identified manually using published codon usage tables, see e.g., Codon Usage Database, supra, (2004), or codon usage tables developed by one skilled in the art. In addition, computer programs designed to assist in the selection of nucleotide changes. Non-limiting examples of such software include eCodonOpt, Gregory L. Moore and Costas D. Maranas, eCodonOpt: A Systematic Computational Framework for Optimizing Codon Usage in Directed Evolution Experiments, 30(11) Nucleic Acids Res. 2407-2416 (2002); DNA Works, see, e.g., David M. Hoover and Jacek Lubkowski, DNAWorks: An Automated Method for Designing Oligonucleotides for PCR-Based Gene Synthesis, 30(10) Nucleic Acids Res. e43 (2002); DNA2.0, see, e.g., Claes Gustafsson et al., Codon Bias and Heterologous Protein Expression, 22(7) Trends Biotechnol. 346-353 (2004); GeMS, see, e.g., Sarah J. Kodumal et al., Total Synthesis of Long DNA Sequences: Synthesis of a Contiguous 32-Kb Polyketide Synthase Gene Cluster, 101(44) Proc. Natl. Acad. Sci. U.S.A. 15573-15578 (2004); CAD PAM, see, e.g., Lance Stewart and Alex B. Burgin, supra, 2005; and Gene Composer, see, e.g., Lance Stewart and Alex B. Burgin, supra, 2005. In addition, publicly available internet sites useful for identifying codon bias are available, such as, Graphical Codon User Analyzer at gcua.schoedl.de, see, e.g., Markus Fuhrmann et al., Monitoring Dynamic Expression of Nuclear Genes in Chlamydomonas Reinhardtii by Using a Synthetic Luciferase Reporter Gene, 55(6) Plant Mol. Biol. 869-881 (2004); and UpGene at URL address vectorcore.pitt.edu/upgene/upgene.html, see, e.g., Wentao Gao et al., UpGene: Application of a Web-based DNA Codon Optimization Algorithm, 20 BIOTECHNOL. PROG. 443-448, (2004). Alternatively, a variety of commercial vendors provide nucleotide optimization services including, but not limited, to Aptagen, Inc. (Herndon, Va.); BLUEHERON® Biotechnology (Bothell, Wash.); deCODE Biostructures, Inc. (Bainbridge Island, Wash.); DNA 2.0 (Menlo Park, Calif.); Entelechon, GmbH. (Regensburg, Germany); Genscript Corp. (Piscataway, N.J.); Modular Genetics, Inc. (Woburn, Mass.); and QIAGEN, Inc. (Valencia, Calif.). The identification of appropriate nucleotide changes to make in a modified open reading frame disclosed in the present specification is a routine procedure within the scope of one skilled in the art and from the teachings herein.

A variety of methods can be used to make a nucleic acid molecule comprising a modified open reading frame disclosed in the present specification, see, e.g., Lance Stewart and Alex B. Burgin, supra, 2005. Non-limiting examples of methods include, oligonucleotide ligation methods, in vivo repair methods and PCR-based methods. The synthesis of nucleic acid molecules is a routine procedure within the scope of one skilled in the art and from the teachings herein.

Nucleic acid synthesis by sequential assembly of complementary oligonucleotides is a solid phase method involving the sequential hybridization of overlapping complementary oligonucleotides to a starting oligonucleotide that is chemically coupled to an insert support, see, e.g., Zdenek Hostomsky and Jiri Smrt, Solid-phase assembly of DNA duplexes from synthetic oligonucleotides, 18 Nucleic Acids Symp Ser. 241-244 (1987); and K L. Beattie and R. F. Fowler, Solid-phase gene assembly, 352(6335) Nature 548-549 (1991). In this oligonucleotide ligation method, oligonucleotide building blocks of approximately 30 nucleotides in length that correspond to the top and bottom strands of the entire gene are individually denatured and purified by denaturing polyacrylamide gel elctrophoresis. These purified oligonucleotides are phosphorylation of the 5′ end, divided into subgroups and then hybridized to form subassemblies on the solid-phase support. Sequential rounds of sub assembly hybridizations to the solid-phase support extend the attached DNA molecule until the full-length gene is constructed.

Nucleic acid synthesis by the FokI method utilizes the E. coli in vivo repair mechanism of DNA synthesis to construct a synthetic gene from oligonucleotides, see e.g., Wlodek Mandecki & Timothy J. Bolling, FokI Method of Gene Synthesis, 68(1) GENE 101-107, (1988), The method is based on the observation that large (approx. 100 bp long) inserts can be cloned into a plasmid using a technique of oligodeoxynucleotide (oligo)-directed double-strand break repair. The method involves transforming a denatured mixture of oligonucleotides of approximately 40 to 90 nucleotides in length and a linearized plasmid into E. coli. The oligonucleotides are designed with terminal sequences which contain a FokI restriction endonuclease site and complement the ends of the linearized plasmid, which also has sites for FokI. The nucleotide (nt) sequences are inserted between the two FokI sites of the plasmid. FokI is a class IIs endonuclease which makes a staggered double strand break at a site 9 and 13 nucleotides away from its recognition site. Upon cleavage of the plasmid DNA with FokI, a restriction fragment is liberated that by design contains unique four nucleotide FokI 5′-overhang sequences that can serve as cohesive ends for subsequent assembly of larger fragments of synthetic DNA until the gene of interest is constructed.

Nucleic acid synthesis by polymerase cycling assembly (PCA) or assembly PCR uses the polymerase chain reaction to construct a gene from oligonucleotides instead of methods involving the ligation of overlapping oligonucleotide, see e.g., Patrick J. Dillon & Craig A. Rosen, A Rapid Method for the Construction of Synthetic Genes Using the Polymerase Chain Reaction, 9(3) BIOTECHNIQUES 298-300, (1990); and Willem P. Stemmer et al., Single-Step Assembly of a Gene and Entire Plasmid from Large Numbers of Oligodeoxyribonucleotides, 164(1) GENE 49-53, (1995). In this method, overlapping, complementary oligonucleotides of approximately 40 to 60 nucleotides in length that correspond to the top and bottom strands of the entire gene are pooled and subjected to multiple cycles of denaturation, renaturation and polymerization. The resulting PCR products are then subjected to PCR amplification using outside flanking primers containing restriction endonuclease sites that facilitate cloning of the final PCR product.

Alternatively, a variety of commercial vendors provide nucleic acid synthesis services through the use of high throughput gene synthesis platforms including, but not limited, to Aptagen, Inc. (Herndon, Va.); BLUEHERON® Biotechnology (Bothell, Wash.); DNA 2.0 (Menlo Park, Calif.); Entelechon, GmbH. (Regensburg, Germany); Genscript Corp. (Piscataway, N.J.); Modular Genetics, Inc. (Woburn, Mass.); and QIAGEN, Inc. (Valencia, Calif.). A method of nucleic acid synthesis is illustrated in Example 3. The synthesis of a modified open reading frame disclosed in the present specification is a routine procedure within the scope of one skilled in the art and from the teachings herein.

Seven antigenically-distinct types of Botulinum toxins (BoNTs) have been identified by investigating botulism outbreaks in man (BoNT/A, /B, /E and /F), animals (BoNT/C1 and /D), or isolated from soil (BoNT/G). BoNTs possess approximately 35% amino acid identity with each other and share the same functional domain organization and overall structural architecture. The amino acid sequences of eight Clostridial toxin serotypes have been derived from the corresponding genes (Niemann, “Molecular Biology of Clostridial Neurotoxins” in Sourcebook of Bacterial Protein Toxins Alouf and Freer (Eds.) pp. 303-348 London: Academic Press 1991). It is recognized by those of skill in the art that within each type of Clostridial toxin there can be various strains differing somewhat in their amino acid sequence, and also in the nucleic acids encoding these proteins. While all seven BoNT serotypes have similar structure and pharmacological properties, each also displays heterogeneous bacteriological characteristics. In contrast, tetanus toxin (TeNT) is produced by a uniform group of C. tetani. Two other species of clostridia, C. baratii and C. butyricum, also produce toxins similar to BoNT/F and BoNT/E, respectively.

Clostridia toxins (CoNTs) are each translated as a single chain polypeptide of approximately 150 kDa that is subsequently cleaved by proteolytic scission within a disulphide loop by bacterial or tissue proteases. This posttranslational processing yields a di-chain molecule comprising an approximately 50 kDa light chain (LC) and an approximately 100 kDa heavy chain (HC) held together by a single disulphide bond and noncovalent interactions. Each mature di-chain molecule comprises three functionally distinct domains: 1) an enzymatic domain located in the LC that includes a metalloprotease region containing a zinc-dependent endopeptidase activity which specifically targets core components of the neurotransmitter release apparatus; 2) a translocation domain contained within the amino-terminal half of the HC(H_{N}) that facilitates release of the toxin from intracellular vesicles into the cytoplasm of the target cell; and 3) a binding domain found within the carboxy-terminal half of the HC(H_{C}) that determines the binding activity and binding specificity of the toxin to the receptor complex located at the surface of the target cell.

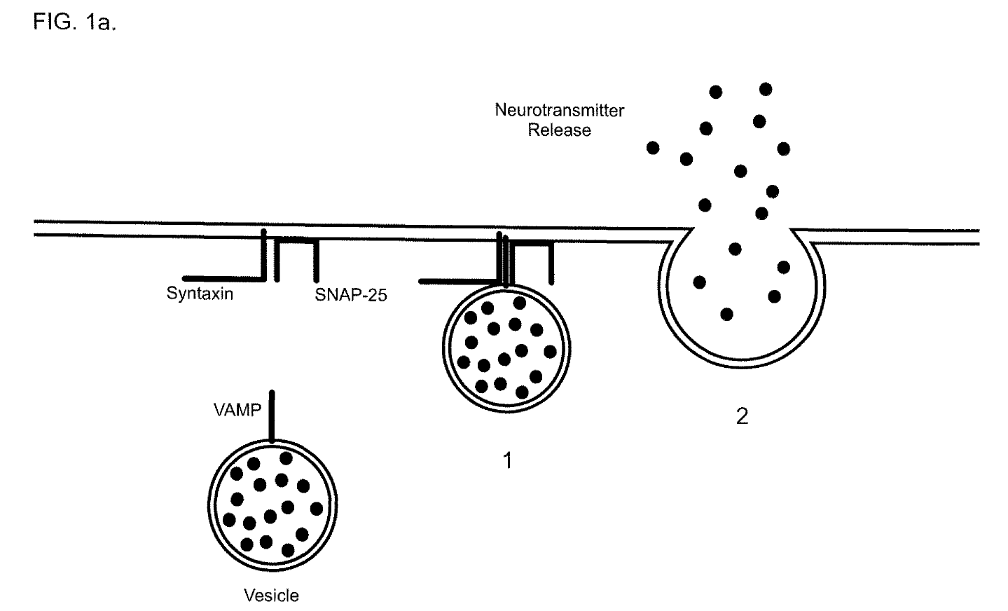

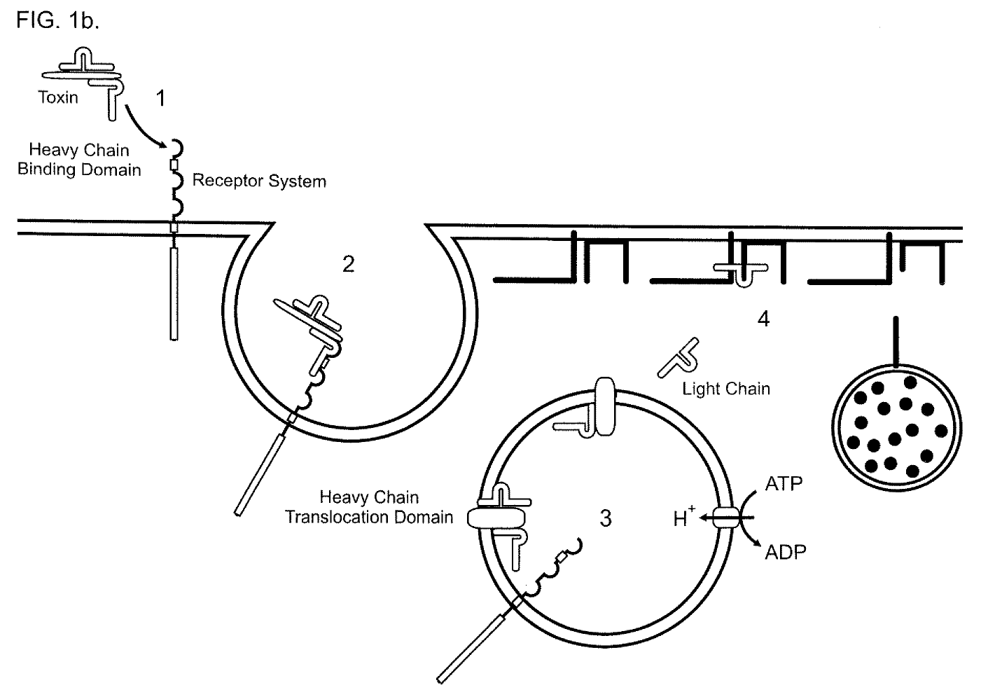

The binding, translocation and enzymatic activity of these three functional domains are all necessary for toxicity. While all details of this process are not yet precisely known, the overall cellular intoxication mechanism whereby CoNTs enter a neuron and inhibit neurotransmitter release is similar, regardless of type. Although the applicants have no wish to be limited by the following description, the intoxication mechanism can be described as comprising at least four steps: 1) receptor binding, 2) complex internalization, 3) light chain translocation, and 4) enzymatic target modification (see FIG. 1). The process is initiated when the H_{C} domain of a CoNT binds to CoNT-specific receptor complex located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the CoNT/receptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote enzymatic activation of the toxin. Once activated, light chain endopeptidase of the toxin is released from the intracellular vesicle into the cytosol where it specifically targets one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxy-terminal region, releasing a nine or twenty-six amino acid segment, respectively, and BoNT/C1 also cleaves SNAP-25 near the carboxy-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. The selective proteolysis of synaptic SNAREs accounts for the total block of neurotransmitter release caused by Clostridial toxins in vivo. The SNARE protein targets of Clostridial toxins are common to exocytosis in a variety of non-neuronal types; in these cells, as in neurons, light chain peptidase activity inhibits exocytosis, see, e.g., Yann Humeau et al., How Botulinum and Tetanus Neurotoxins Block Neurotransmitter Release, 82(5) Biochimie. 427-446 (2000); Kathryn Turton et al., Botulinum and Tetanus Neurotoxins: Structure, Function and Therapeutic Utility, 27(11) Trends Biochem. Sci. 552-558. (2002); M. Zouhair Atassi, Basic and Therapeutic Aspects of Botulinum and Tetanus Toxins, (Dirk W. Dressler & Joseph J. Jankovic eds., 2003); Giovanna Lalli et al., The Journey of Tetanus and Botulinum Neurotoxins in Neurons, 11(9) Trends Microbiol. 431-437, (2003) which are hereby incorporated by reference.

Aspects of the present invention provide, in part, an active BoNT/E. As used herein, the term “active BoNT/E” means any protein, or fragment thereof, that can execute the overall cellular mechanism whereby BoNT/E enter a neuron and inhibit neurotransmitter release and encompasses the binding of a BoNT/E to a low or high affinity receptor complex, the internalization of the toxin/receptor complex, the translocation of the BoNT/E light chain into the cytoplasm and the enzymatic modification of a BoNT/E substrate. Thus, active BoNT/E encompass without limitation, naturally occurring active BoNT/E variants, such as, e.g., active BoNT/E isoforms, non-naturally occurring active BoNT/E variants, such as, e.g., conservative BoNT/E variants, non-conservative BoNT/E variants and active BoNT/E fragments thereof, or any combination thereof. As used herein, the term “BoNT/E variant,” whether naturally-occurring or non-naturally-occurring, means an active BoNT/E that has at least one amino acid change from the corresponding region of SEQ ID NO: 1 and can be described in percent identity to the corresponding region of SEQ ID NO: 1. As a non-limiting example, an active BoNT/E variant comprising amino acids 1-1252 of SEQ ID NO: 1 will have at least one amino acid difference, such as, e.g., an amino acid substitution, deletion or addition, as compared to the amino acid region 1-1252 of SEQ ID NO: 1. As another non-limiting example, an active BoNT/E variant comprising amino acids 15-1240 of SEQ ID NO: 1 will have at least one amino acid difference, such as, e.g., an amino acid substitution, deletion or addition, as compared to the amino acid region 15-1240 of SEQ ID NO: 1.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: improving the sensitivity of progressive multiple sequence alignment through sequence weighting, position-specific gap penalties and weight matrix choice, 22(22) Nucleic Acids Research 4673-4680 (1994); and iterative refinement, see, e.g., Osamu Gotoh, Significant improvement in accuracy of multiple protein sequence alignments by iterative refinement as assessed by reference to structural alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-box: a fundamentally new algorithm for the simultaneous alignment of several protein sequences, 8(5) CABIOS 501-509 (1992); Gibbs sampling, see, e.g., C. E. Lawrence et al., Detecting subtle sequence signals: a gibbs sampling strategy for multiple alignment, 262(5131) Science 208-214 (1993); Align-M, see, e.g., Ivo Van Walle et al., Align-m—a new algorithm for multiple alignment of highly divergent sequences, 20(9) Bioinformatics: 1428-1435 (2004).

Hybrid methods combine functional aspects of both global and local alignment methods. Non-limiting methods include, e.g., segment-to-segment comparison, see, e.g., Burkhard Morgenstern et al., Multiple DNA and protein sequence alignment based on segment-to-segment comparison, 93(22) Proc. Natl. Acad. Sci. U.S.A. 12098-12103 (1996); T-Coffee, see, e.g., Cédric Notredame et al., T-Coffee: a novel algorithm for multiple sequence alignment, 302(1) J. Mol. Biol. 205-217 (2000); MUSCLE, see, e.g., Robert C. Edgar, MUSCLE: Multiple sequence alignment with high score accuracy and high throughput, 32(5) Nucleic Acids Res. 1792-1797 (2004); and DIALIGN-T, see, e.g., Amarendran R Subramanian et al., DIALIGN-T: An improved algorithm for segment-based multiple sequence alignment, 6(1) BMC Bioinformatics 66 (2005).

As used herein, the term “naturally occurring BoNT/E variant” means any active BoNT/E produced without the aid of any human manipulation, including, without limitation, BoNT/E isoforms produced from alternatively-spliced transcripts and BoNT/E isoforms produced by spontaneous mutation. As used herein, the term “non-naturally occurring BoNT/E variant” means any active BoNT/E produced with the aid of human manipulation, including, without limitation, active BoNT/E produced by genetic engineering using random mutagenesis or rational designed and active BoNT/E produced by chemical synthesis.

As used herein, the term “conservative BoNT/E variant” means an active BoNT/E that has at least one amino acid substituted by another amino acid or an amino acid analog that has at least one property similar to that of the original amino acid. Examples of properties include, without limitation, similar size, topography, charge, hydrophobicity, hydrophilicity, lipophilicity covalent-bonding capacity, hydrogen-bonding capacity, a physicochemically property, of the like, or any combination thereof. A conservative BoNT/E variant can function in substantially the same manner as the active BoNT/E on which the conservative BoNT/E variant is based, and can be substituted for the active BoNT/E in any aspect of the present invention. A conservative BoNT/E variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the active BoNT/E on which the DAGL conservative variant is based. A conservative BoNT/E variant can also substitute at least 10 contiguous amino acids, at least 15 contiguous amino acids, at least 20 contiguous amino acids, or at least 25 contiguous amino acids from the active BoNT/E on which the conservative BoNT/E variant is based, that possess at least 50% amino acid identity, 65% amino acid identity, 75% amino acid identity, 85% amino acid identity or 95% amino acid identity to the active BoNT/E on which the conservative BoNT/E variant is based.

As used herein, the term “non-conservative BoNT/E variant” means an active BoNT/E in which 1) at least one amino acid is deleted from the active BoNT/E on which the non-conservative BoNT/E variant is based; 2) at least one amino acid added to the active BoNT/E on which the non-conservative BoNT/E variant is based; or 3) at least one amino acid is substituted by another amino acid or an amino acid analog that does not share any property similar to that of the original amino acid. A non-conservative BoNT/E variant can function in substantially the same manner as the active BoNT/E on which the non-conservative BoNT/E variant is based, and can be substituted for the active BoNT/E in any aspect of the present invention. A non-conservative BoNT/E variant can delete one or more amino acids, two or more amino acids, three or more amino acids, four or more amino acids, five or more amino acids, and ten or more amino acids from the active BoNT/E on which the non-conservative BoNT/E variant is based. A non-conservative BoNT/E variant can add one or more amino acids, two or more amino acids, three or more amino acids, four or more amino acids, five or more amino acids, and ten or more amino acids to the active BoNT/E on which the non-conservative BoNT/E variant is based. A non-conservative BoNT/E variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the active BoNT/E on which the non-conservative BoNT/E variant is based. A non-conservative BoNT/E variant can also substitute at least 10 contiguous amino acids, at least 15 contiguous amino acids, at least 20 contiguous amino acids, or at least 25 contiguous amino acids from the active BoNT/E on which the non-conservative BoNT/E variant is based, that possess at least 50% amino acid identity, 65% amino acid identity, 75% amino acid identity, 85% amino acid identity or 95% amino acid identity to the active BoNT/E on which the non-conservative BoNT/E variant is based.

It is also envisioned that any of a variety of active BoNT/E fragments can be useful in aspects of the present invention with the proviso that these active fragments can execute the overall cellular mechanism whereby an active BoNT/E proteolytically cleaves a substrate. Thus, aspects of this embodiment can include active BoNT/E fragments having a length of, e.g., at least 300 amino acids, at least 400 amino acids, at least 500 amino acids, at least 600 amino acids, at least 700 amino acids, at least 800 amino acids, at least 900 amino acids, at least 1000 amino acids, at least 1100 amino acids and at least 1200 amino acids. Other aspects of this embodiment, can include active BoNT/E fragments having a length of, e.g., at most 300 amino acids, at most 400 amino acids, at most 500 amino acids, at most 600 amino acids, at most 700 amino acids, at most 800 amino acids, at most 900 amino acids, at most 1000 amino acids, at most 1100 amino acids and at most 1200 amino acids.

Thus, in an embodiment, a nucleic acid molecule comprising a modified open reading frame disclosed in the present specification encodes an active BoNT/E. Other aspects of this embodiment include, without limitation, naturally occurring BoNT/E variants, such as, e.g., BoNT/E isoforms, non-naturally occurring BoNT/E variants, such as, e.g., conservative BoNT/E variants, non-conservative BoNT/E variants and active BoNT/E fragments, or any combination thereof. In another embodiment, a nucleic acid molecule comprising a modified open reading frame disclosed in the present specification encodes an active BoNT/E comprising SEQ ID NO:1. Other aspects of this embodiment include, without limitation, naturally occurring BoNT/E variants of SEQ ID NO: 1, such as, e.g., BoNT/E isoforms of SEQ ID NO: 1, non-naturally occurring BoNT/E variants of SEQ ID NO: 1, such as, e.g., conservative BoNT/E variants of SEQ ID NO: 1, non-conservative BoNT/E variants of SEQ ID NO: 1 and active BoNT/E fragments of SEQ ID NO: 1, or any combination thereof.

In still other aspects of this embodiment, an active BoNT/E has, e.g., at least 70% amino acid identity with SEQ ID NO:1, at least 75% amino acid identity with the SEQ ID NO:1, at least 80% amino acid identity with SEQ ID NO:1, at least 85% amino acid identity with SEQ ID NO:1, at least 90% amino acid identity with SEQ ID NO:1 or at least 95% amino acid identity with SEQ ID NO:1. In yet other aspects of this embodiment, an active BoNT/E has, e.g., at most 70% amino acid identity with SEQ ID NO:1, at most 75% amino acid identity with the SEQ ID NO:1, at most 80% amino acid identity with SEQ ID NO:1, at most 85% amino acid identity with SEQ ID NO:1, at most 90% amino acid identity with SEQ ID NO:1 or at most 95% amino acid identity with SEQ ID NO:1.

In other aspects of this embodiment, an active BoNT/E has, e.g., at most one, two, three, four, five, six, seven, eight, nine, 10, 20, 30, 40 or 50 non-contiguous amino acid substitutions relative to SEQ ID NO:1. In other aspects of this embodiment, an active BoNT/E has, e.g., at least one, two, three, four, five, six, seven, eight, nine, 10, 20, 30, 40 or 50 non-contiguous amino acid substitutions relative to SEQ ID NO:1. In yet other aspects of this embodiment, an active BoNT/E has, e.g., at most one, two, three, four, five, six, seven, eight, nine, 10, 20, 30, 40 or 50 non-contiguous amino acid deletions relative to SEQ ID NO:1. In other aspects of this embodiment, an active BoNT/E has, e.g., at least one, two, three, four, five, six, seven, eight, nine, 10, 20, 30, 40 or 50 non-contiguous amino acid deletions relative to SEQ ID NO:1. In still other aspects of this embodiment, an active BoNT/E has, e.g., at most one, two, three, four, five, six, seven, eight, nine, 10, 20, 30, 40 or 50 non-contiguous amino acid additions relative to SEQ ID NO:1. In other aspects of this embodiment, an active BoNT/E has, e.g., at least one, two, three, four, five, six, seven, eight, nine, 10, 20, 30, 40 or 50 non-contiguous amino acid additions relative to SEQ ID NO:1.

In other aspects of this embodiment, an active BoNT/E has, e.g., at most one, two, three, four, five, six, seven, eight, nine, 10, 20, 30, 40 or 50 contiguous amino acid substitutions relative to SEQ ID NO:1. In other aspects of this embodiment, an active BoNT/E has, e.g., at least one, two, three, four, five, six, seven, eight, nine, 10, 20, 30, 40 or 50 contiguous amino acid substitutions relative to SEQ ID NO:1. In yet other aspects of this embodiment, an active BoNT/E has, e.g., at most one, two, three, four, five, six, seven, eight, nine, 10, 20, 30, 40 or 50 contiguous amino acid deletions relative to SEQ ID NO:1. In other aspects of this embodiment, an active BoNT/E has, e.g., at least one, two, three, four, five, six, seven, eight, nine, 10, 20, 30, 40 or 50 contiguous amino acid deletions relative to SEQ ID NO:1. In still other aspects of this embodiment, an active BoNT/E has, e.g., at most one, two, three, four, five, six, seven, eight, nine, 10, 20, 30, 40 or 50 contiguous amino acid additions relative to SEQ ID NO:1. In other aspects of this embodiment, an active BoNT/E has, e.g., at least one, two, three, four, five, six, seven, eight, nine, 10, 20, 30, 40 or 50 contiguous amino acid additions relative to SEQ ID NO:1.

Aspects of the present invention provide, in part, a heterologous cell. As used herein, the term “heterologous cell” means any cell other than the native strain of Clostridium from which the Clostridial toxin was discovered. that expresses, or can be engineered to express an active BoNT/E disclosed in the present specification. Thus, for example, a heterologous cell that expresses a nucleic acid molecule comprising a modified open reading frame encoding an active BoNT/E would be any prokaryotic or eukaryotic cell other than the C. botulinum strain that produces the E serotype. The term heterologous cell encompasses cells from a variety of organisms, including, without limitation, bacteria strains, yeast strains, plant cells and cell lines derived from plants, insect cells and cell lines derived from insects and mammalian cells and cell lines derived from mammals. It is understood that cells useful in aspects of the invention can included, without limitation, primary cells; cultured cells; established cells; normal cells; transformed cells; tumor cells; infected cells; proliferating and terminally differentiated cells; and stably or transiently transfected cells, including stably and transiently transfected cells. It is further understood that cells useful in aspects of the invention can be in any state such as proliferating or quiescent; intact or permeabilized such as through chemical-mediated transfection such as, e.g., calcium phosphate-mediated, diethyl-laminoethyl (DEAE) dextran-mediated, lipid-mediated, polyethyleneimine (PEI)-mediated and polybrene-mediated; physical-mediated tranfection, such as, e.g., biolistic particle delivery, microinjection and electroporation; and viral-mediated transfection, such as, e.g., retroviral-mediated transfection. It is further understood that cells useful in aspects of the invention may include those which express an active BoNT/E under control of a constitutive, tissue-specific, cell-specific or inducible promoter element, enhancer element or both.

Because a wide variety of factors could influence the selection of a specific heterologous cell, nucleic acid molecules comprising a modified open reading frame providing increased expression of the encoded active BoNT/E can be designed to be expressed in a range of prokaryotic and eukaryotic cells. Codon usage tables and G+C content information for prokaryotic and eukaryotic organisms are publicly maintained by the Codon Usage Database, The First Laboratory for Plant Gene Research, Kazusa DNA Research Institute (2004).

Thus in an embodiment, nucleic acid molecules comprising a modified open reading frame providing increased expression of the encoded active BoNT/E are expressed in a prokaryotic cell. Non-limiting examples of prokaryotic cells include strains of aerobic, microaerophilic, capnophilic, facultative, anaerobic, gram-negative and gram-positive bacterial cells such as those derived from, e.g., Escherichia coli, Salmonella typhimurium, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls and Neisseria meningitidis. In an aspect of this embodiment, a nucleic acid molecule disclosed in the present specification is expressed in an E. coli strain. In other aspects of this embodiment, a nucleic acid molecule is expressed in an E. coli strain comprises, e.g., the open reading frame of SEQ ID NO: 4, SEQ ID NO: 5, SEQ ID NO: 6, SEQ ID NO: 7, SEQ ID NO: 117, SEQ ID NO: 122 or SEQ ID NO: 124. In an aspect of this embodiment, a nucleic acid molecule disclosed in the present specification is expressed in a B. fragilis strain. In other aspects of this embodiment, a nucleic acid molecule expressed in a B. fragilis strain comprises, e.g., the open reading frame of SEQ ID NO: 8, SEQ ID NO: 9 or SEQ ID NO: 10. In an aspect of this embodiment, a nucleic acid molecule disclosed in the present specification is expressed in a B. licheniformis strain. In other aspects of this embodiment, a nucleic acid molecule expressed in a B. licheniformis strain comprises, e.g., the open reading frame of SEQ ID NO: 11, SEQ ID NO: 12 or SEQ ID NO: 13. In an aspect of this embodiment, a nucleic acid molecule disclosed in the present specification is expressed in a B. subtilis strain. In other aspects of this embodiment, a nucleic acid molecule expressed in an B. subtilis strain comprises, e.g., the open reading frame of SEQ ID NO: 14, SEQ ID NO: 15 or SEQ ID NO: 16. In an aspect of this embodiment, a nucleic acid molecule disclosed in the present specification is expressed in a C. difficile strain. In another aspect of this embodiment, a nucleic acid molecule expressed in a C. difficile strain comprises, e.g., the open reading frame of SEQ ID NO: 17, SEQ ID NO: 18 or SEQ ID NO: 19. In an aspect of this embodiment, a nucleic acid molecule disclosed in the present specification is expressed in a C. perfringens strain. In other aspects of this embodiment, a nucleic acid molecule expressed in a C. perfringens strain comprises, e.g., the open reading frame of SEQ ID NO: 20, SEQ ID NO: 21 or SEQ ID NO: 22. In an aspect of this embodiment, a nucleic acid molecule disclosed in the present specification is expressed in a C. crescentus strain. In other aspects of this embodiment, a nucleic acid molecule expressed in a C. crescentus strain comprises, e.g., the open reading frame of SEQ ID NO: 23, SEQ ID NO: 24 or SEQ ID NO: 25. In an aspect of this embodiment, a nucleic acid molecule disclosed in the present specification is expressed in a L. lactis strain. In another aspect of this embodiment, a nucleic acid molecule expressed in a L. lactis strain comprises, e.g., the open reading frame of SEQ ID NO: 26, SEQ ID NO: 27 or SEQ ID NO: 28. In an aspect of this embodiment, a nucleic acid molecule disclosed in the present specification is expressed in a M. extorquens strain. In another aspect of this embodiment, a nucleic acid molecule expressed in a M. extorquens strain comprises, e.g., the open reading frame of SEQ ID NO: 29, SEQ ID NO: 30 or SEQ ID NO: 31. In an aspect of this embodiment, a nucleic acid molecule disclosed in the present specification is expressed in an N. meningirulls strain. In an aspect of this embodiment, a nucleic acid molecule disclosed in the present specification is expressed in a S. typhimurium strain. In other aspects of this embodiment, a nucleic acid molecule expressed in a S. typhimurium strain comprises, e.g., the open reading frame of SEQ ID NO: 32, SEQ ID NO: 33 or SEQ ID NO: 34.

In another embodiment, nucleic acid molecules comprising a modified open reading frame providing increased expression of the encoded active BoNT/E is expressed in an eukaryotic cell or cell line derived from an eukaryotic cell. In aspects of this embodiment, a nucleic acid molecule expressed in an eukaryotic cell or cell line derived from an eukaryotic cell comprises, e.g., any one of the open reading frames of SEQ ID NO: 35 through SEQ ID NO: 97.

In yet another embodiment, nucleic acid sequence molecules comprising a modified open reading frame providing increased expression of the encoded active BoNT/E is expressed in a yeast strain. Non-limiting examples of yeast strains include those derived from, e.g., Pichia pastoris, Pichia methanolica, Pichia angusta, Schizosaccharomyces pombe, Saccharomyces cerevisiae and Yarrowia lipolytica. In an aspect of this embodiment, a nucleic acid molecule disclosed in the present specification is expressed in a P. pastoris strain. In other aspects of this embodiment, a nucleic acid molecule expressed in a P. pastoris strain comprises, e.g., the open reading frame of SEQ ID NO: 35, SEQ ID NO: 36 or SEQ ID NO: 37. In an aspect of this embodiment, a nucleic acid molecule disclosed in the present specification is expressed in a P. methanolica strain. In other aspects of this embodiment, a nucleic acid molecule expressed in a P. methanolica strain comprises, e.g., the open reading frame of SEQ ID NO: 35, SEQ ID NO: 36 or SEQ ID NO: 37. In an aspect of this embodiment, a nucleic acid molecule disclosed in the present specification is expressed in a P. angusta strain. In other aspects of this embodiment, a nucleic acid molecule expressed in a P. angusta strain comprises, e.g., the open reading frame of SEQ ID NO: 35, SEQ ID NO: 36 or SEQ ID NO: 37. In an aspect of this embodiment, a nucleic acid molecule disclosed in the present specification is expressed in a S. cerevisiae strain. In other aspects of this embodiment, a nucleic acid molecule expressed in a S. cerevisiae strain comprises, e.g., the open reading frame of SEQ ID NO: 38, SEQ ID NO: 39 or SEQ ID NO: 40. In an aspect of this embodiment, a nucleic acid molecule disclosed in the present specification is expressed in a S. pombe strain. In other aspects of this embodiment, a nucleic acid molecule expressed in a S. pombe strain comprises, e.g., the open reading frame of SEQ ID NO: 41, SEQ ID NO: 42 or SEQ ID NO: 43. In an aspect of this embodiment, a nucleic acid molecule disclosed in the present specification is expressed in a Y. lipolytica strain. In other aspects of this embodiment, a nucleic acid molecule expressed in a Y. lipolytica strain comprises, e.g., the open reading frame of SEQ ID NO: 44, SEQ ID NO: 45 or SEQ ID NO: 46.

In yet another embodiment, nucleic acid sequence molecules comprising a modified open reading frame providing increased expression of the encoded active BoNT/E is expressed in a slime mold strain. Non-limiting examples of slime mold strains include those derived from, e.g., Dictyostelium discoideum. In an aspect of this embodiment, a nucleic acid molecule disclosed in the present specification is expressed in a D. discoideum strain. In other aspects of this embodiment, a nucleic acid molecule expressed in a D. discoideum strain comprises, e.g., the open reading frame of SEQ ID NO: 47, SEQ ID NO: 48 or SEQ ID NO: 49.

In yet another embodiment, nucleic acid sequence molecules comprising a modified open reading frame providing increased expression of the encoded active BoNT/E is expressed in a plant cell. Non-limiting examples of plant cells and cell lines derived from plant cells include those derived from, e.g., species of monocots, such as, e.g., Zea mays and species of dicots, such as, e.g., Arabidopsis thaliana, Lemna gibba and Lemna minor. In an aspect of this embodiment, a nucleic acid molecule disclosed in the present specification is expressed in a monocot cell or cell line derived from a monocot cell. In an aspect of this embodiment, a nucleic acid molecule disclosed in the present specification is expressed in a dicot cell or cell line derived from a dicot cell. In an aspect of this embodiment, a nucleic acid molecule disclosed in the present specification is expressed in a Z. mays cell or cell line derived from a Z. mays cell. In other aspects of this embodiment, a nucleic acid molecule expressed in a Z. mays cell or cell line derived from a Z. mays cell comprises, e.g., the open reading frame of SEQ ID NO: 50, SEQ ID NO: 51 or SEQ ID NO: 52. In an aspect of this embodiment, a nucleic acid molecule disclosed in the present specification is expressed in an A. thaliana cell or cell line derived from an A. thaliana cell. In other aspects of this embodiment, a nucleic acid molecule expressed in an A. thaliana cell or cell line derived from an A. thaliana cell comprises, e.g., the open reading frame of SEQ ID NO: 53, SEQ ID NO: 54 or SEQ ID NO: 55.

In yet another embodiment, nucleic acid sequence molecules comprising a modified open reading frame providing increased expression of the encoded active BoNT/E is expressed in an insect cell or a cell line derived from insects. Non-limiting examples of insect cells and cell lines derived from insects such as those derived from, e.g., Spodoptera frugiperda, Trichoplusia ni, Drosophila melanogaster and Manduca sexta. In an aspect of this embodiment, a nucleic acid molecule disclosed in the present specification is expressed in a D. melanogaster cell or a cell line derived from D. melanogaster. In other aspects of this embodiment, a nucleic acid molecule expressed in a D. melanogaster cell or a cell line derived from D. melanogaster comprises, e.g., the open reading frame of SEQ ID NO: 56, SEQ ID NO: 57 or SEQ ID NO: 58. In an aspect of this embodiment, a nucleic acid molecule disclosed in the present specification is expressed in a S. frugiperda strain or a cell line derived from S. frugiperda. In other aspects of this embodiment, a nucleic acid molecule expressed in a S. frugiperda cell or a cell line derived from S. frugiperda comprises, e.g., the open reading frame of SEQ ID NO: 59, SEQ ID NO: 60 or SEQ ID NO: 61. In an aspect of this embodiment, a nucleic acid molecule disclosed in the present specification is expressed in a T. ni cell or a cell line derived from T. ni. In other aspects of this embodiment, a nucleic acid molecule expressed in a T. ni cell or a cell line derived from T. ni comprises, e.g., the open reading frame of SEQ ID NO: 59, SEQ ID NO: 60 or SEQ ID NO: 61. In an aspect of this embodiment, a nucleic acid molecule disclosed in the present specification is expressed in a M. sexta strain or a cell line derived from M. sexta. In an aspect of this embodiment, a nucleic acid molecule disclosed in the present specification is expressed in a Sf9 cell line. In other aspects of this embodiment, a nucleic acid molecule expressed in a Sf9 cell line comprises, e.g., the open reading frame of SEQ ID NO: 59, SEQ ID NO: 60 or SEQ ID NO: 61. In an aspect of this embodiment, a nucleic acid molecule disclosed in the present specification is expressed in a Sf21 cell line. In other aspects of this embodiment, a nucleic acid molecule expressed in a Sf21 cell line comprises, e.g., the open reading frame of SEQ ID NO: 59, SEQ ID NO: 60 or SEQ ID NO: 61. In an aspect of this embodiment, a nucleic acid molecule disclosed in the present specification is expressed in a High-Five cell line. In other aspects of this embodiment, a nucleic acid molecule expressed in a High-Five cell line comprises, e.g., the open reading frame of SEQ ID NO: 59, SEQ ID NO: 60 or SEQ ID NO: 61. In an aspect of this embodiment, a nucleic acid molecule disclosed in the present specification is expressed in a Schneider's Drosophila line 2 (S2) cell line. In other aspects of this embodiment, a nucleic acid molecule expressed in a Schneider's Drosophila line 2 (S2) cell line comprises, e.g., the open reading frame of SEQ ID NO: 56, SEQ ID NO: 57 or SEQ ID NO: 58. In an aspect of this embodiment, a nucleic acid molecule disclosed in the present specification is expressed in a Kc cell line. In other aspects of this embodiment, a nucleic acid molecule expressed in a Kc cell line comprises, e.g., the open reading frame of SEQ ID NO: 56, SEQ ID NO: 57 or SEQ ID NO: 58.

In yet another embodiment, nucleic acid sequence molecules comprising a modified open reading frame providing increased expression of the encoded active BoNT/E is expressed in a fish cell or a cell line derived from a fish cell. Non-limiting examples of fish cells and cell lines derived from fish cells include those derived from, e.g., Danio rerio. In an aspect of this embodiment, a nucleic acid molecule disclosed in the present specification is expressed in a D. rerio cell or a cell line derived from D. rerio. In other aspects of this embodiment, a nucleic acid molecule expressed in a D. rerio cell or a cell line derived from D. rerio comprises, e.g., the open reading frame of SEQ ID NO: 62, SEQ ID NO: 63 or SEQ ID NO: 64.

In yet another embodiment, nucleic acid sequence molecules comprising a modified open reading frame providing increased expression of the encoded active BoNT/E is expressed in an amphibian cell. Non-limiting examples of amphibian cells and cell lines derived from amphibian cells include those derived from, e.g., Xenopus. In an aspect of this embodiment, a nucleic acid molecule disclosed in the present specification is expressed in a X. laevis cell or a cell line derived from X. laevis. In other aspects of this embodiment, a nucleic acid molecule expressed in a X. laevis cell or a cell line derived from X. laevis comprises, e.g., the open reading frame of SEQ ID NO: 65, SEQ ID NO: 66 or SEQ ID NO: 67. In another aspect of this embodiment, a nucleic acid molecule disclosed in the present specification is expressed in a X. tropicalis cell or a cell line derived from X. tropicalis. In other aspects of this embodiment, a nucleic acid molecule expressed in a X. tropicalis cell or a cell line derived from X. tropicalis comprises, e.g., the open reading frame of SEQ ID NO: 68, SEQ ID NO: 69 or SEQ ID NO: 70.

In yet another embodiment, nucleic acid sequence molecules comprising a modified open reading frame providing increased expression of the encoded active BoNT/E is expressed in a bird cell. Non-limiting examples of bird cells and cell lines derived from bird cells include those derived from, e.g., Gallus gallus. In an aspect of this embodiment, a nucleic acid molecule disclosed in the present specification is expressed in a G. gallus cell or a cell line derived from G. gallus. In other aspects of this embodiment, a nucleic acid molecule expressed in a G. gallus cell or a cell line derived from G. gallus comprises, e.g., the open reading frame of SEQ ID NO: 71, SEQ ID NO: 72 or SEQ ID NO: 73.

In yet another embodiment, nucleic acid sequence molecules comprising a modified open reading frame providing increased expression of the encoded active BoNT/E is expressed in a mammalian cell. Non-limiting examples of mammalian cells and cell lines derived from mammalian cells include those derived from, e.g., mouse, rat, hamster, porcine, bovine, equine, primate and human. In an aspect of this embodiment, a nucleic acid molecule disclosed in the present specification is expressed in a mouse cell or a cell line derived from mouse. In other aspects of this embodiment, a nucleic acid molecule expressed in a mouse cell or a cell line derived from mouse comprises, e.g., the open reading frame of SEQ ID NO: 74, SEQ ID NO: 75 or SEQ ID NO: 76. In yet another aspect of this embodiment, a nucleic acid molecule disclosed in the present specification is expressed in a Mus musculus cell or a cell line derived from M. musculus. In yet other aspects of this embodiment, a nucleic acid molecule expressed in a M. musculus cell or a cell line derived from M. musculus comprises, e.g., the open reading frame of SEQ ID NO: 74, SEQ ID NO: 75 or SEQ ID NO: 76. In an aspect of this embodiment, a nucleic acid molecule disclosed in the present specification is expressed in a 10T1/2 cell line. In other aspects of this embodiment, a nucleic acid molecule expressed in a 10T1/2 cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a BALB/3T3 cell line. In yet other aspects of this embodiment, a nucleic acid molecule expressed in a BALB/3T3 cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a L-M cell line. In yet other aspects of this embodiment, a nucleic acid molecule expressed in a L-M cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a NB4 cell line. In yet other aspects of this embodiment, a nucleic acid molecule expressed in a NB4 cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a 1A3 cell line. In yet other aspects of this embodiment, a nucleic acid molecule expressed in a 1A3 cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a NIE-115 cell line. In yet other aspects of this embodiment, a nucleic acid molecule expressed in a NIE-115 cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a NG108-15 cell line. In yet other aspects of this embodiment, a nucleic acid molecule expressed in a NG108-15 cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a NIH3T3 cell line. In yet other aspects of this embodiment, a nucleic acid molecule expressed in a NIH3T3 cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a NCTC cell line. In yet other aspects of this embodiment, a nucleic acid molecule expressed in a NCTC cell line comprises, e.g., the open reading frame of SEQ ID NO: 74, SEQ ID NO: 75 or SEQ ID NO: 76. In another aspect of this embodiment, a nucleic acid molecule disclosed in the present specification is expressed in a Neuro-2A cell line. In yet other aspects of this embodiment, a nucleic acid molecule expressed in a Neuro-2A cell line comprises, e.g., the open reading frame of SEQ ID NO: 74, SEQ ID NO: 75 or SEQ ID NO: 76.

In an aspect of this embodiment, a nucleic acid molecule disclosed in the present specification is expressed in a rat cell or a cell line derived from rat. In other aspects of this embodiment, a nucleic acid molecule expressed in a rat cell or a cell line derived from rat comprises, e.g., the open reading frame of SEQ ID NO: 77, SEQ ID NO: 78 or SEQ ID NO: 79. In yet another aspect of this embodiment, a nucleic acid molecule disclosed in the present specification is expressed in a Rattus norvegicus cell or a cell line derived from R. norvegicus. In yet another aspect of this embodiment, a nucleic acid molecule expressed in a R. norvegicus cell or a cell line derived from R. norvegicus comprises, e.g., the open reading frame of SEQ ID NO: 77, SEQ ID NO: 78 or SEQ ID NO: 79. In an aspect of this embodiment, a nucleic acid molecule disclosed in the present specification is expressed in a PC12 cell line. In other aspects of this embodiment, a nucleic acid molecule expressed in a PC12 cell line comprises, e.g., the open reading frame of SEQ ID NO: 77, SEQ ID NO: 78 or SEQ ID NO: 79. In another aspect of this embodiment, a nucleic acid molecule disclosed in the present specification is expressed in a GH1 cell line. In yet other aspects of this embodiment, a nucleic acid molecule expressed in a GH1 cell line comprises, e.g., the open reading frame of SEQ ID NO: 77, SEQ ID NO: 78 or SEQ ID NO: 79. In another aspect of this embodiment, a nucleic acid molecule disclosed in the present specification is expressed in a GH3 cell line. In yet other aspects of this embodiment, a nucleic acid molecule expressed in a GH3 cell line comprises, e.g., the open reading frame of SEQ ID NO: 77, SEQ ID NO: 78 or SEQ ID NO: 79. In another aspect of this embodiment, a nucleic acid molecule disclosed in the present specification is expressed in a C6 cell line. In yet other aspects of this embodiment, a nucleic acid molecule expressed in a C6 cell line comprises, e.g., the open reading frame of SEQ ID NO: 77, SEQ ID NO: 78 or SEQ ID NO: 79. In another aspect of this embodiment, a nucleic acid molecule disclosed in the present specification is expressed in a L2 cell line. In yet other aspects of this embodiment, a nucleic acid molecule expressed in a L2 cell line comprises, e.g., the open reading frame of SEQ ID NO: 77, SEQ ID NO: 78 or SEQ ID NO: 79.

In an aspect of this embodiment, a nucleic acid molecule disclosed in the present specification is expressed in a hamster cell or a cell line derived from hamster. In other aspects of this embodiment, a nucleic acid molecule expressed in a hamster cell or a cell line derived from hamster comprises, e.g., the open reading frame of SEQ ID NO: 80, SEQ ID NO: 81 or SEQ ID NO: 82. In yet another aspect of this embodiment, a nucleic acid molecule disclosed in the present specification is expressed in a Cricetulus griseus cell or a cell line derived from C. griseus. In yet other aspects of this embodiment, a nucleic acid molecule expressed in a C. griseus cell or a cell line derived from C. griseus comprises, e.g., the open reading frame of SEQ ID NO: 80, SEQ ID NO: 81 or SEQ ID NO: 82. In an aspect of this embodiment, a nucleic acid molecule disclosed in the present specification is expressed in a CHO cell line. In other aspects of this embodiment, a nucleic acid molecule expressed in a CHO cell line comprises, e.g., the open reading frame of SEQ ID NO: 80, SEQ ID NO: 81 or SEQ ID NO: 82. In another aspect of this embodiment, a nucleic acid molecule disclosed in the present specification is expressed in a 6E6 cell line. In yet other aspects of this embodiment, a nucleic acid molecule expressed in a 6E6 cell line comprises, e.g., the open reading frame of SEQ ID NO: 80, SEQ ID NO: 81 or SEQ ID NO: 82.

In an aspect of this embodiment, a nucleic acid molecule disclosed in the present specification is expressed in a porcine cell or a cell line derived from porcine. In other aspects of this embodiment, a nucleic acid molecule expressed in a porcine cell or a cell line derived from porcine comprises, e.g., the open reading frame of SEQ ID NO: 83, SEQ ID NO: 84 or SEQ ID NO: 85. In yet another aspect of this embodiment, a nucleic acid molecule disclosed in the present specification is expressed in a Sus scrofa cell or a cell line derived from S. scrofa. In yet other aspects of this embodiment, a nucleic acid molecule expressed in a S. scrofa cell or a cell line derived from S. scrofa comprises, e.g., the open reading frame of SEQ ID NO: 83, SEQ ID NO: 84 or SEQ ID NO: 85. In an aspect of this embodiment, a nucleic acid molecule disclosed in the present specification is expressed in a PK15 cell line. In other aspects of this embodiment, a nucleic acid molecule expressed in a PK15 cell line comprises, e.g., the open reading frame of SEQ ID NO: 83, SEQ ID NO: 84 or SEQ ID NO: 85. In another aspect of this embodiment, a nucleic acid molecule disclosed in the present specification is expressed in a LLC-PK1 cell line. In yet other aspects of this embodiment, a nucleic acid molecule expressed in a LLC-PK1 cell line comprises, e.g., the open reading frame of SEQ ID NO: 83, SEQ ID NO: 84 or SEQ ID NO: 85. In another aspect of this embodiment, a nucleic acid molecule disclosed in the present specification is expressed in a ST cell line. In yet other aspects of this embodiment, a nucleic acid molecule expressed in a ST cell line comprises, e.g., the open reading frame of SEQ ID NO: 83, SEQ ID NO: 84 or SEQ ID NO: 85. In another aspect of this embodiment, a nucleic acid molecule disclosed in the present specification is expressed in a ESK-4 cell line. In yet other aspects of this embodiment, a nucleic acid molecule expressed in a ESK-4 cell line comprises, e.g., the open reading frame of SEQ ID NO: 83, SEQ ID NO: 84 or SEQ ID NO: 85.

In an aspect of this embodiment, a nucleic acid molecule disclosed in the present specification is expressed in a bovine cell or a cell line derived from bovine. In other aspects of this embodiment, a nucleic acid molecule expressed in a bovine cell or a cell line derived from bovine comprises, e.g., the open reading frame of SEQ ID NO: 86, SEQ ID NO: 87 or SEQ ID NO: 88. In yet another aspect of this embodiment, a nucleic acid molecule disclosed in the present specification is expressed in a Bos taurus cell or a cell line derived from B. taurus. In yet other aspects of this embodiment, a nucleic acid molecule expressed in a B. taurus cell or a cell line derived from B. taurus comprises, e.g., the open reading frame of SEQ ID NO: 86, SEQ ID NO: 87 or SEQ ID NO: 88. In an aspect of this embodiment, a nucleic acid molecule disclosed in the present specification is expressed in a CPAE cell line. In other aspects of this embodiment, a nucleic acid molecule expressed in a CPAE cell line comprises, e.g., the open reading frame of SEQ ID NO: 86, SEQ ID NO: 87 or SEQ ID NO: 88. In another aspect of this embodiment, a nucleic acid molecule disclosed in the present specification is expressed in a BT cell line. In yet other aspects of this embodiment, a nucleic acid molecule expressed in a BT cell line comprises, e.g., the open reading frame of SEQ ID NO: 86, SEQ ID NO: 87 or SEQ ID NO: 88. In another aspect of this embodiment, a nucleic acid molecule disclosed in the present specification is expressed in a SBAC cell line. In yet other aspects of this embodiment, a nucleic acid molecule expressed in a SBAC cell line comprises, e.g., the open reading frame of SEQ ID NO: 86, SEQ ID NO: 87 or SEQ ID NO: 88. In another aspect of this embodiment, a nucleic acid molecule disclosed in the present specification is expressed in a FB2 cell line. In yet other aspects of this embodiment, a nucleic acid molecule expressed in a FB2 cell line comprises, e.g., the open reading frame of SEQ ID NO: 86, SEQ ID NO: 87 or SEQ ID NO: 88.

In an aspect of this embodiment, a nucleic acid molecule disclosed in the present specification is expressed in a equine cell or a cell line derived from equine. In other aspects of this embodiment, a nucleic acid molecule expressed in a equine cell or a cell line derived from equine comprises, e.g., the open reading frame of SEQ ID NO: 89, SEQ ID NO: 90 or SEQ ID NO: 91. In yet another aspect of this embodiment, a nucleic acid molecule disclosed in the present specification is expressed in a Equus caballus cell or a cell line derived from E. caballus. In yet other aspects of this embodiment, a nucleic acid molecule expressed in a E. caballus cell or a cell line derived from E. caballus comprises, e.g., the open reading frame of SEQ ID NO: 89, SEQ ID NO: 90 or SEQ ID NO: 91. In an aspect of this embodiment, a nucleic acid molecule disclosed in the present specification is expressed in a NBL-6 cell line. In other aspects of this embodiment, a nucleic acid molecule expressed in a NBL-6 cell line comprises, e.g., the open reading frame of SEQ ID NO: 89, SEQ ID NO: 90 or SEQ ID NO: 91.

In an aspect of this embodiment, a nucleic acid molecule disclosed in the present specification is expressed in a primate cell or a cell line derived from primate. In other aspects of this embodiment, a nucleic acid molecule expressed in a primate cell or a cell line derived from primate comprises, e.g., the open reading frame of SEQ ID NO: 92, SEQ ID NO: 93 or SEQ ID NO: 94. In yet another aspect of this embodiment, a nucleic acid molecule disclosed in the present specification is expressed in a Cercopithecus aethiops cell or a cell line derived from C. aethiops. In yet other aspects of this embodiment, a nucleic acid molecule expressed in a C. aethiops cell or a cell line derived from C. aethiops comprises, e.g., the open reading frame of SEQ ID NO: 92, SEQ ID NO: 93 or SEQ ID NO: 94. In an aspect of this embodiment, a nucleic acid molecule disclosed in the present specification is expressed in a COS-1 cell line. In other aspects of this embodiment, a nucleic acid molecule expressed in a COS-1 cell line comprises, e.g., the open reading frame of SEQ ID NO: 92, SEQ ID NO: 93 or SEQ ID NO: 94. In another aspect of this embodiment, a nucleic acid molecule disclosed in the present specification is expressed in a COS-7 cell line. In yet other aspects of this embodiment, a nucleic acid molecule expressed in a COS-7 cell line comprises, e.g., the open reading frame of SEQ ID NO: 92, SEQ ID NO: 93 or SEQ ID NO: 94. In another aspect of this embodiment, a nucleic acid molecule disclosed in the present specification is expressed in a VV-1 cell line. In yet other aspects of this embodiment, a nucleic acid molecule expressed in a VV-1 cell line comprises, e.g., the open reading frame of SEQ ID NO: 92, SEQ ID NO: 93 or SEQ ID NO: 94.

In an aspect of this embodiment, a nucleic acid molecule disclosed in the present specification is expressed in a human cell or a cell line derived from human. In another aspect of this embodiment, a nucleic acid molecule expressed in a human cell or a cell line derived from human comprises, e.g., the open reading frame of SEQ ID NO: 95, SEQ ID NO: 96 or SEQ ID NO: 97. In yet another aspect of this embodiment, a nucleic acid molecule disclosed in the present specification is expressed in a Homo sapiens cell or a cell line derived from H. sapiens. In another aspect of this embodiment, a nucleic acid molecule expressed in a H. sapiens cell or a cell line derived from H. sapiens comprises, e.g., the open reading frame of SEQ ID NO: 95, SEQ ID NO: 96 or SEQ ID NO: 97. In an aspect of this embodiment, a nucleic acid molecule disclosed in the present specification is expressed in a SH-SY5Y cell line. In other aspects of this embodiment, a nucleic acid molecule expressed in a SH-SY5Y cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a SK-N-DZ cell line. In yet other aspects of this embodiment, a nucleic acid molecule expressed in a SK-N-DZ cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a SK-N-SH cell line. In yet other aspects of this embodiment, a nucleic acid molecule expressed in a SK-N-SH cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a BE(2)-C cell line. In yet other aspects of this embodiment, a nucleic acid molecule expressed in a BE(2)-C cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a HeLa cell line. In yet other aspects of this embodiment, a nucleic acid molecule expressed in a HeLa cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a HEK 293 cell line. In yet other aspects of this embodiment, a nucleic acid molecule expressed in a HEK 293 cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a MCF-7 cell line. In yet other aspects of this embodiment, a nucleic acid molecule expressed in a MCF-7 cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a HepG2 cell line. In yet other aspects of this embodiment, a nucleic acid molecule expressed in a HepG2 cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a HL-60 cell line. In yet other aspects of this embodiment, a nucleic acid molecule expressed in a HL-60 cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a IMR-32 cell line. In yet other aspects of this embodiment, a nucleic acid molecule expressed in a IMR-32 cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a SW-13 cell line. In yet other aspects of this embodiment, a nucleic acid molecule expressed in a SW-13 cell line comprises, e.g., the open reading frame of SEQ ID NO: 95, SEQ ID NO: 96 or SEQ ID NO: 97. In another aspect of this embodiment, a nucleic acid molecule disclosed in the present specification is expressed in a CHP3 cell line. In yet other aspects of this embodiment, a nucleic acid molecule expressed in a CHP3 cell line comprises, e.g., the open reading frame of SEQ ID NO: 95, SEQ ID NO: 96 or SEQ ID NO: 97.

The nucleic acid molecules disclosed in the present specification include, in part, a modified open reading frame providing increased expression of an encoded active BoNT/E. Increased expression of an active BoNT/E is determined by comparing the amount of an active BoNT/E expressed from a modified open reading frame with the amount of the same active BoNT/E expressed from an unmodified open reading frame in an otherwise identical nucleic acid molecule. As used herein, the term “modified open reading frame” means an open reading frame that contains at least one nucleotide change providing increased expression of the encoded active BoNT/E. As used herein, the term “unmodified open reading frame” means an open reading frame that does not contain any nucleotide changes providing increased expression of the encoded active BoNT/E. As a non-limiting example, SEQ ID NO: 3 and SEQ ID NO: 98 are unmodified open reading frames that will not provide increased expression of the encoded active BoNT/E in a heterologous cell and SEQ ID NO: 4 through SEQ ID NO: 97, SEQ ID NO: 117, SEQ ID NO: 122 and SEQ ID NO: 124 are modified open reading frames that can provide increased expression of the encoded active BoNT/E in the appropriate heterologous cell. It is further understood by one skilled in the art that the methods and procedures used to express the nucleic acid molecules comprising the modified open reading frame should be the same or similar to the methods and procedures used to express the nucleic acid molecules comprising the unmodified open reading frame to ensure accurate and consistent comparisons.

A wide variety of well-established methods can be used to compare the amount of expressed active BoNT/E from a modified open reading frame to the amount of the same active BoNT/E expressed from an unmodified open reading frame in an otherwise identical nucleic acid molecule. Comparisons of amounts of an active BoNT/E expressed can be either qualitative or quantitative.

Active BoNT/E amounts can be measured using any procedure that can separate and visualize proteins from a cell lystae, such as, e.g., procedures involving gel electrophoresis and protein staining, western blotting, protein-labeling, as well as, other procedures involving protein separation and visualization. Thus, amounts of active BoNT/E can be appraised by labeling active BoNT/E using a radioactive amino acid tracer and visualizing expression by autoradiography after gel electrophoresis. Likewise, incorporation of radiolabeled amino acids into active BoNT/E can be measured by scintillation counting after Trichloroacetic Acid (TCA) precipitation. Amounts of active BoNT/E can also be assessed by staining proteins separated by gel electrophoresis using, e.g., dye staining procedures like Coomassie Brilliant Blue and Colloidal Coomassie Brilliant Blue; fluorescence staining procedures like SYPRO® Ruby and ruthenium II; or silver staining procedures. Amounts of active BoNT/E can likewise be determined by antibody staining after Western blot analysis. Furthermore, functional assays that measure the biological activity of active BoNT/E can be used to compare amounts of active BoNT/E expressed from a modified open reading frame to the amount of the same active BoNT/E expressed from an unmodified open reading frame in an otherwise identical nucleic acid molecule, such as, e.g., SNAP25 cleavage assay and the GFP-SNAP25 Fluorescence Release Assay. Non-limiting examples of specific procedures to separate and visualize protein amounts, as well as well-characterized reagents, conditions and protocols are readily available from commercial vendors that include, without limitation, Amersham Biosciences, Piscataway, N.J.; Bio-Rad Laboratories, Hercules, Calif.; Pierce Biotechnology, Inc., Rockford, Ill.; Promega Corporation, Madison, Wis., and Stratagene, La Jolla, Calif. In addition, non-limiting examples of specific protocols necessary to separate, visualize and quantify a protein are described in e.g., MOLECULAR CLONING A LABORATORY MANUAL, supra, (2001); and CURRENT PROTOCOLS IN MOLECULAR BIOLOGY, supra, (2004). These protocols are routine procedures within the scope of one skilled in the art and from the teaching herein.

Active BoNT/E amounts can be measured after one or more purification steps using, without limitation, gel electrophoresis and protein staining, western blotting, protein-labeling, UV absorbance, the Lowry assay, the biuret assay, the Smith copper/bicinchoninic (BCA) assay, and the Bradford dye assay, see e.g., Christine V. Sapan et al., Colorimetric Protein Assay Techniques, 29(2) BIOTECHNOL. APPL. BIOCHEM. 99-108, (1999). Any of a variety of methods can be used for purifying an active BoNT/E disclosed in the present specification. Examples of purification methods include, without limitation, ammonium sulfate or ethanol precipitation, acid extraction, ion exchange chromatography, phosphocellulose chromatography, lectin chromatography, affinity chromatography, hydrophobic interaction chromatography, size exclusion chromatography, gel-filtration chromatography, adsorption chromatography, hydroxyapatite chromatography, fast performance liquid chromatography (FPLC), and high performance liquid (HPLC) chromatography. Binding moieties of the target peptide of interest may be attached to any of a variety of substances including, without limitation resins, agarose, and magnetic beads. In addition, any of a variety of processing techniques can be used including, without limitation, batch-wise processing, and gravity-feed columns. Protein refolding steps may also be necessary to ensure recovery of a functionally active BoNT/E encoded by nucleic acid molecules disclosed in the specification. Non-limiting examples of specific protocols for purifying and recovering proteins are described in, e.g., John Abelson et al., GUIDE TO PROTEIN PURIFICATION, (Academic Press, 1990), PROTEIN PURIFICATION: PRINCIPLES AND PRACTICE, (Robert K. Scopes et al. eds., Springer Verlag, 3^{rd} ed. 1994), PROTEIN PURIFICATION TECHNIQUES: A PRACTICAL APPROACH, (Simon Roe ed., Oxford University Press, 2^{nd} ed. 2001), MOLECULAR CLONING A LABORATORY MANUAL, supra, (2001), Ian M. Rosenberg, PROTEIN ANALYSIS & PURIFICATION: BENCHTOP TECHNIQUES, (Springer Verlag, 2002). These protocols are routine procedures within the scope of one skilled in the art and from the teaching herein.

Thus, in an embodiment, the amount of an active BoNT/E expressed from a modified open reading frame is increased as compared to the amount of the same active BoNT/E expressed from an unmodified open reading frame in an otherwise identical nucleic acid molecule. In aspects of this embodiment, the amount of an active BoNT/E expressed from a modified open reading frame is, e.g., increased at least 1.5-fold as compared to the amount of the same BoNT/E expressed from an unmodified open reading frame in an otherwise identical nucleic acid molecule; increased at least 2-fold as compared to the amount of the same BoNT/E expressed from an unmodified open reading frame in an otherwise identical nucleic acid molecule; increased at least 3-fold as compared to the amount of the same BoNT/E expressed from an unmodified open reading frame in an otherwise identical nucleic acid molecule; increased at least 4-fold as compared to the amount of the same BoNT/E expressed from an unmodified open reading frame in an otherwise identical nucleic acid molecule; increased at least 5-fold as compared to the amount of the same BoNT/E expressed from an unmodified open reading frame in an otherwise identical nucleic acid molecule; increased at least 10-fold as compared to the amount of the same BoNT/E expressed from an unmodified open reading frame in an otherwise identical nucleic acid molecule; increased at least 25-fold as compared to the amount of the same BoNT/E expressed from an unmodified open reading frame in an otherwise identical nucleic acid molecule; increased at least 50-fold as compared to the amount of the same BoNT/E expressed from an unmodified open reading frame in an otherwise identical nucleic acid molecule; increased at least 100-fold as compared to the amount of the same BoNT/E expressed from an unmodified open reading frame in an otherwise identical nucleic acid molecule; or increased at least 200-fold as compared to the amount of the same BoNT/E expressed from an unmodified open reading frame in an otherwise identical nucleic acid molecule.

In aspects of this embodiment, the amount of an active BoNT/E expressed from a modified open reading frame is, e.g., increased at most 1.5-fold as compared to the amount of the same BoNT/E expressed from an unmodified open reading frame in an otherwise identical nucleic acid molecule; increased at most 2-fold as compared to the amount of the same BoNT/E expressed from an unmodified open reading frame in an otherwise identical nucleic acid molecule; increased at most 3-fold as compared to the amount of the same BoNT/E expressed from an unmodified open reading frame in an otherwise identical nucleic acid molecule; increased at most 4-fold as compared to the amount of the same BoNT/E expressed from an unmodified open reading frame in an otherwise identical nucleic acid molecule; increased at most 5-fold as compared to the amount of the same BoNT/E expressed from an unmodified open reading frame in an otherwise identical nucleic acid molecule; increased at most 10-fold as compared to the amount of the same BoNT/E expressed from an unmodified open reading frame in an otherwise identical nucleic acid molecule; increased at most 25-fold as compared to the amount of the same BoNT/E expressed from an unmodified open reading frame in an otherwise identical nucleic acid molecule; increased at most 50-fold as compared to the amount of the same BoNT/E expressed from an unmodified open reading frame in an otherwise identical nucleic acid molecule; increased at most 100-fold as compared to the amount of the same BoNT/E expressed from an unmodified open reading frame in an otherwise identical nucleic acid molecule; or increased at most 200-fold as compared to the amount of the same BoNT/E expressed from an unmodified open reading frame in an otherwise identical nucleic acid molecule.

Other aspects of the present invention provide expression constructs comprising a nucleic acid molecule disclosed in the present specification, operably-linked to an expression vector useful for expressing the nucleic acid molecule in a heterologous cell. A wide variety of expression vectors are envisioned, including, without limitation, a prokaryotic expression vector useful for expressing a nucleic acid molecule comprising a modified open reading frame providing increased expression of the encoded active BoNT/E in a prokaryotic cell; a yeast expression vector useful for expressing a nucleic acid molecule comprising a modified open reading frame providing increased expression of the encoded active BoNT/E in a yeast cell; an insect expression vector useful for expressing a nucleic acid molecule comprising a modified open reading frame providing increased expression of the encoded active BoNT/E in an insect cell; a mammalian expression vector useful for expressing a nucleic acid molecule comprising a modified open reading frame providing increased expression of the encoded active BoNT/E in a mammalian cell.

The expression constructs disclosed in the present specification include, in part, a nucleic acid molecule. In is envisioned that any and all nucleic acid molecules disclosed in the present specification can be used. Thus, aspects of this embodiment include, without limitation, nucleic acid molecules comprising a modified open reading frame providing increased expression of the encoded active BoNT/E in a prokaryotic cell; nucleic acid molecules comprising a modified open reading frame providing increased expression of the encoded active BoNT/E in a yeast cell; nucleic acid molecules comprising a modified open reading frame providing increased expression of the encoded active BoNT/E in an slime mold cell; nucleic acid molecules comprising a modified open reading frame providing increased expression of the encoded active BoNT/E in a plant cell or cell line derived from a plant cell; nucleic acid molecules comprising a modified open reading frame providing increased expression of the encoded active BoNT/E in an insect cell or cell line derived from an insect cell; nucleic acid molecules comprising a modified open reading frame providing increased expression of the encoded active BoNT/E in an fish cell or cell line derived from a fish cell; nucleic acid molecules comprising a modified open reading frame providing increased expression of the encoded active BoNT/E in an amphibian cell or cell line derived from an amphibian cell; nucleic acid molecules comprising a modified open reading frame providing increased expression of the encoded active BoNT/E in a bird cell or cell line derived from a bird cell; and nucleic acid molecules comprising a modified open reading frame providing increased expression of the encoded active BoNT/E in a mammalian cell or cell line derived from a mammalian cell, such as, e.g., mouse, rat, hamster, porcine, bovine, equine, primate and human.

The expression constructs disclosed in the present specification include, in part, a heterologous cell. In is envisioned that any and all heterologous cells disclosed in the present specification can be used. Thus, aspects of this embodiment include, without limitation, prokaryotic cells prokaryotic cells including, without limitation, strains of aerobic, microaerophilic, capnophilic, facultative, anaerobic, gram-negative and gram-positive bacterial cells such as those derived from, e.g., Escherichia coli, Salmonella typhimurium, Bacillus subtilis, Bacillus licheniformis, Clostridia perfringens, Clostridia difficile, Bacteroides fragilis, Caulobacter crescentus, Methylobacterium extorquens, Lactococcus lactis and Neisseria meningirulls; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; slime mold strains, such as, e.g., those derived from, e.g., Dictyostelium discoideum; plant cells and cell lines derived from plant cells, such as, e.g., those derived from species of monocots, species of dicots, Zea mays and Arabidopsis thaliana; insect cells and cell lines derived from insects, such as, e.g., those derived from Spodoptera frugiperda, Trichoplusia ni, Drosophila melanogaster and Manduca sexta; fish cells and cell lines derived from fish cells, such as, e.g., those derived from Denio renia; amphibian cells and cell lines derived from amphibian cells, such as, e.g., those derived from Xenopus laevis and Xenopus tropicalis; bird cells and cell lines derived from bird cells, such as, e.g., those derived from Gallus gallus; mammalian cells and cell lines derived from mammalian cells, such as, e.g., those derived from mouse, rat, hamster, porcine, bovine, equine, primate and human.

The expression constructs disclosed in the present specification include, in part, a nucleic acid molecule disclosed in the present specification, operably-linked to an expression vector. As used herein, the term “operably linked” means any of a variety of cloning methods that can join a nucleic acid molecule disclosed in the present specification to an expression vector such that a peptide encoded by the nucleic acid molecule is expressed when introduced into a heterologous cell. Well-established molecular biology techniques that may be necessary to make an expression construct disclosed in the present specification including, but not limited to, procedures involving polymerase chain reaction (PCR) amplification restriction enzyme reactions, agarose gel electrophoresis, nucleic acid ligation, bacterial transformation, nucleic acid purification, nucleic acid sequencing are routine procedures well within the scope of one skilled in the art and from the teaching herein. Non-limiting examples of specific protocols necessary to make an expression construct are described in e.g., MOLECULAR CLONING A LABORATORY MANUAL, supra, (2001); and CURRENT PROTOCOLS IN MOLECULAR BIOLOGY (Frederick M. Ausubel et al., eds. John Wiley & Sons, 2004), which are hereby incorporated by reference. These protocols are routine procedures well within the scope of one skilled in the art and from the teaching herein.

A wide variety of expression vectors can be employed for expressing an open reading frame encoding an active BoNT/E and include without limitation, viral expression vectors, prokaryotic expression vectors and eukaryotic expression vectors including yeast, insect, plant and mammalian expression vectors. Non-limiting examples of expression vectors, along with well-established reagents and conditions for making and using an expression construct from such expression vectors are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; EMD Biosciences-Novagen, Madison, Wis.; QIAGEN, Inc., Valencia, Calif.; and Stratagene, La Jolla, Calif. The selection, making and use of an appropriate expression vector are routine procedures well within the scope of one skilled in the art and from the teachings herein.

It is envisioned that any of a variety of expression systems may be useful for expressing constructs disclosed in the present specification. An expression system encompasses both cell-based systems and cell-free expression systems. Cell-based systems include, without limited, viral expression systems, prokaryotic expression systems, yeast expression systems, baculoviral expression systems, insect expression systems and mammalian expression systems. Cell-free systems include, without limitation, wheat germ extracts, rabbit reticulocyte extracts and E. coli extracts and generally are equivalent to the method disclosed herein. Expression using an expression system can include any of a variety of characteristics including, without limitation, inducible expression, non-inducible expression, constitutive expression, viral-mediated expression, stably-integrated expression, and transient expression. Expression systems that include well-characterized vectors, reagents, conditions and cells are well-established and are readily available from commercial vendors that include, without limitation, Ambion, Inc. Austin, Tex.; BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; Roche Applied Science, Indianapolis, Ind.; and Stratagene, La Jolla, Calif. Non-limiting examples on the selection and use of appropriate heterologous expression systems are described in e.g., PROTEIN EXPRESSION. A PRACTICAL APPROACH(S. J. Higgins and B. David Hames eds., Oxford University Press, 1999); Joseph M. Fernandez & James P. Hoeffler, GENE EXPRESSION SYSTEMS. USING NATURE FOR THE ART OF EXPRESSION (Academic Press, 1999); and Meena Rai & Harish Padh, Expression Systems for Production of Heterologous Proteins, 80(9) CURRENT SCIENCE 1121-1128, (2001), which are hereby incorporated by reference. These protocols are routine procedures well within the scope of one skilled in the art and from the teaching herein.

Thus, in an embodiment disclosed in the present invention, a nucleic acid molecule disclosed in the present specification is operably linked to control sequences from a viral expression vector useful for expressing an encoded active BoNT/E in a viral expression system. Non-limiting examples of viral expression vector include lentivirus vectors, fowl pox virus, pseudorabies virus, retrovirus vectors, semliki forest virus vectors, sindbis virus vectors, vaccinia virus vectors, and adenovirus vectors. In an aspect of this embodiment, an expression construct comprises a viral expression vector operably linked to a modified open reading frame providing increased expression of an encoded active BoNT/E in a mammalian cell.

In another embodiment disclosed in the present invention, a nucleic acid molecule disclosed in the present specification is operably linked to control sequences from a prokaryotic expression vector useful for expressing an encoded active BoNT/E in a prokaryotic cell. Non-limiting examples of prokaryotic expression vectors include an Escherichia coli expression vector, a Salmonella typhimurium expression vector, a Caulobacter crescentus expression vector, a Methylobacterium extorquens expression vector, a Lactococcus lactis expression vector, a Neisseria meningirulls expression vector, a Bacillus subtilis expression vector and a Bacillus licheniformis expression vector. In an aspect of this embodiment, an expression construct comprises a prokaryotic expression vector operably linked to a modified open reading frame providing increased expression of an encoded active BoNT/E in a prokaryotic cell. In an aspect of this embodiment, an expression construct comprises a pET28 expression vector and a modified open reading frame providing increased expression of an encoded active BoNT/E in an E. coli cell. In another aspect of this embodiment, an expression construct comprises a pET28 expression vector operably linked to a modified open reading frame of SEQ ID NO: 4 providing increased expression of the encoded active BoNT/E in an E. coli cell. In another aspect of this embodiment, an expression construct comprises a pET28 expression vector operably linked to a modified open reading frame of SEQ ID NO: 5 providing increased expression of the encoded active BoNT/E in an E. coli cell. In another aspect of this embodiment, an expression construct comprises a pET28 expression vector operably linked to a modified open reading frame of SEQ ID NO: 6 providing increased expression of the encoded active BoNT/E in an E. coli cell. In another aspect of this embodiment, an expression construct comprises a pET28 expression vector operably linked to a modified open reading frame of SEQ ID NO: 7 providing increased expression of the encoded active BoNT/E in an E. coli cell. In another aspect of this embodiment, an expression construct comprises a pET29 expression vector operably linked to a modified open reading frame of SEQ ID NO: 4 providing increased expression of the encoded active BoNT/E in an E. coli cell. In another aspect of this embodiment, an expression construct comprises a pET29 expression vector operably linked to a modified open reading frame of SEQ ID NO: 5 providing increased expression of the encoded active BoNT/E in an E. coli cell. In another aspect of this embodiment, an expression construct comprises a pET29 expression vector operably linked to a modified open reading frame of SEQ ID NO: 6 providing increased expression of the encoded active BoNT/E in an E. coli cell. In another aspect of this embodiment, an expression construct comprises a pET29 expression vector operably linked to a modified open reading frame of SEQ ID NO: 7 providing increased expression of the encoded active BoNT/E in an E. coli cell. In another aspect of this embodiment, an expression construct comprises a pRSET expression vector operably linked to a modified open reading frame of SEQ ID NO: 4 providing increased expression of the encoded active BoNT/E in an E. coli cell. In another aspect of this embodiment, an expression construct comprises a pRSET expression vector operably linked to a modified open reading frame of SEQ ID NO: 5 providing increased expression of the encoded active BoNT/E in an E. coli cell. In another aspect of this embodiment, an expression construct comprises a pRSET expression vector operably linked to a modified open reading frame of SEQ ID NO: 6 providing increased expression of the encoded active BoNT/E in an E. coli cell. In another aspect of this embodiment, an expression construct comprises a pRSET expression vector operably linked to a modified open reading frame of SEQ ID NO: 7 providing increased expression of the encoded active BoNT/E in an E. coli cell.

In yet another embodiment disclosed in the present invention, expression constructs disclosed in the present specification are operably linked to control sequences from a eukaryotic expression vector useful for expressing an encoded active BoNT/E in an eukaryotic cell. In an aspect of this embodiment, a nucleic acid molecule disclosed in the present specification is operably linked to control sequences from a yeast expression vector useful for expressing an encoded BoNT/E in a yeast cell. Non-limiting examples of yeast expression vectors include a Pichia pastoris expression vector, a Pichia methanolica expression vector, a Pichia angusta expression vector, a Schizosaccharomyces pombe expression vector, a Saccharomyces cerevisiae expression vector and a Yarrowia lipolytica expression vector. In an aspect of this embodiment, an expression construct comprises a yeast expression vector operably linked to a modified open reading frame providing increased expression of an encoded active BoNT/E in a yeast cell. In an aspect of this embodiment, an expression construct comprises a pPICZ A expression vector and a modified open reading frame providing increased expression of an encoded active BoNT/E in a P. pastoris cell. In another aspect of this embodiment, an expression construct comprises a pPICZ A expression vector operably linked to a modified open reading frame of SEQ ID NO: 35 providing increased expression of the encoded active BoNT/E in a P. pastoris cell. In another aspect of this embodiment, an expression construct comprises a pPICZ A expression vector operably linked to a modified open reading frame of SEQ ID NO: 36 providing increased expression of the encoded active BoNT/E in a P. pastoris cell. In another aspect of this embodiment, an expression construct comprises a pPICZ A expression vector operably linked to a modified open reading frame of SEQ ID NO: 37 providing increased expression of the encoded active BoNT/E in a P. pastoris cell.

In yet another aspect of this embodiment, a nucleic acid molecule disclosed in the present specification is operably linked to control sequences from an insect expression vector useful for expressing an encoded active BoNT/E in an insect cell. Non-limiting examples of an insect expression vector include a Spodoptera frugiperda expression vector, a Trichoplusia ni expression vector, a Drosophila melanogaster expression vector and a Manduca sexta expression vector. In an aspect of this embodiment, an expression construct comprises an insect expression vector operably linked to a modified open reading frame providing increased expression of an encoded active BoNT/E in an insect cell or cell line derived from an insect cell. In an aspect of this embodiment, an expression construct comprises a pFastBac™HT expression vector and a modified open reading frame providing increased expression of an encoded active BoNT/E in an insect cell line, such as, e.g., Sf9, Sf21 and High-Five. In another aspect of this embodiment, an expression construct comprises a pFastBac™HT expression vector operably linked to a modified open reading frame of SEQ ID NO: 59 providing increased expression of the encoded active BoNT/E in an insect cell line, such as, e.g., Sf9, Sf21 and High-Five. In another aspect of this embodiment, an expression construct comprises a pFastBac™HT expression vector operably linked to a modified open reading frame of SEQ ID NO: 60 providing increased expression of the encoded active BoNT/E in an insect cell line, such as, e.g., Sf9, Sf21 and High-Five. In another aspect of this embodiment, an expression construct comprises a pFastBac™HT expression vector operably linked to a modified open reading frame of SEQ ID NO: 61 providing increased expression of the encoded active BoNT/E in an insect cell line, such as, e.g., Sf9, Sf21 and High-Five.

In an aspect of this embodiment, an expression construct comprises a pMT/BiP-V5-His/GFP expression vector and a modified open reading frame providing increased expression of an encoded active BoNT/E in an insect cell line, such as, e.g., Schneider's Drosophila line 2 (S2) and Kc. In another aspect of this embodiment, an expression construct comprises a pMT/BiP-V5-His/GFP expression vector operably linked to a modified open reading frame of SEQ ID NO: 56 providing increased expression of the encoded active BoNT/E in an insect cell line, such as, e.g., Schneider's Drosophila line 2 (S2) and Kc. In another aspect of this embodiment, an expression construct comprises a pMT/BiP-V5-His/GFP expression vector operably linked to a modified open reading frame of SEQ ID NO: 57 providing increased expression of the encoded active BoNT/E in an insect cell line, such as, e.g., Schneider's Drosophila line 2 (S2) and Kc. In another aspect of this embodiment, an expression construct comprises a pMT/BiP-V5-His/GFP expression vector operably linked to a modified open reading frame of SEQ ID NO: 58 providing increased expression of the encoded active BoNT/E in an insect cell line, such as, e.g., Schneider's Drosophila line 2 (S2) and Kc.

In yet another aspect of this embodiment, a nucleic acid molecule disclosed in the present specification is operably linked to control sequences from a mammalian expression vector useful for expressing an encoded active BoNT/E in a mammalian cell or cell line derived from a mammalian cell. Non-limiting examples of mammalian expression vectors include a mouse expression vector, a rat expression vector, a hamster expression vector, a porcine expression vector, a bovine expression vector, an equine expression vector, a primate expression vector and a human expression vector. In an aspect of this embodiment, an expression construct comprises a mammalian expression vector operably linked to a modified open reading frame providing increased expression of an encoded active BoNT/E in a mammalian cell or cell line derived from a mammalian cell.

In an aspect of this embodiment, an expression construct comprises a mouse expression vector operably linked to a modified open reading frame providing increased expression of an encoded active BoNT/E in a mouse cell or cell line derived from a mouse cell. In an aspect of this embodiment, an expression construct comprises a pQBI25fC1 expression vector and a modified open reading frame providing increased expression of an encoded active BoNT/E in a mouse cell line, such as, e.g., 10T1/2, BALB/3T3, L-M, NB4 1A3, NIE-115, NG108-15, NIH3T3, NCTC and Neuro 2A. In another aspect of this embodiment, an expression construct comprises a pQBI25fC1 expression vector operably linked to a modified open reading frame of SEQ ID NO: 74 providing increased expression of the encoded active BoNT/E in a mouse cell line, such as, e.g., 10T1/2, BALB/3T3, L-M, NB4 1A3, NIE-115, NG108-15, NIH3T3, NCTC and Neuro 2A. In another aspect of this embodiment, an expression construct comprises a pQBI25fC1 expression vector operably linked to a modified open reading frame of SEQ ID NO: 75 providing increased expression of the encoded active BoNT/E in a mouse cell line, such as, e.g., 10T1/2, BALB/3T3, L-M, NB4 1A3, NIE-115, NG108-15, NIH3T3, NCTC and Neuro 2A. In another aspect of this embodiment, an expression construct comprises a pQBI25fC1 expression vector operably linked to a modified open reading frame of SEQ ID NO: 76 providing increased expression of the encoded active BoNT/E in a mouse cell line, such as, e.g., 10T1/2, BALB/3T3, L-M, NB4 1A3, NIE-115, NG108-15, NIH3T3, NCTC and Neuro 2A.

In an aspect of this embodiment, an expression construct comprises a rat expression vector operably linked to a modified open reading frame providing increased expression of an encoded active BoNT/E in a rat cell or cell line derived from a rat cell. In an aspect of this embodiment, an expression construct comprises a pQBI25fC1 expression vector and a modified open reading frame providing increased expression of an encoded active BoNT/E in a rat cell line, such as, e.g., PC12, GH1, GH3, C6 and L2. In another aspect of this embodiment, an expression construct comprises a pQBI25fC1 expression vector operably linked to a modified open reading frame of SEQ ID NO: 77 providing increased expression of the encoded active BoNT/E in a rat cell line, such as, e.g., PC12, GH1, GH3, C6 and L2. In another aspect of this embodiment, an expression construct comprises a pQBI25fC1 expression vector operably linked to a modified open reading frame of SEQ ID NO: 78 providing increased expression of the encoded active BoNT/E in a rat cell line, such as, e.g., PC12, GH1, GH3, C6 and L2. In another aspect of this embodiment, an expression construct comprises a pQBI25fC1 expression vector operably linked to a modified open reading frame of SEQ ID NO: 79 providing increased expression of the encoded active BoNT/E in a rat cell line, such as, e.g., PC12, GH1, GH3, C6 and L2.

In an aspect of this embodiment, an expression construct comprises a hamster expression vector operably linked to a modified open reading frame providing increased expression of an encoded active BoNT/E in a hamster cell or cell line derived from a hamster cell. In an aspect of this embodiment, an expression construct comprises a pQBI25fC1 expression vector and a modified open reading frame providing increased expression of an encoded active BoNT/E in a hamster cell line, such as, e.g., CHO and 6E6. In another aspect of this embodiment, an expression construct comprises a pQBI25fC1 expression vector operably linked to a modified open reading frame of SEQ ID NO: 80 providing increased expression of the encoded active BoNT/E in a hamster cell line, such as, e.g., CHO and 6E6. In another aspect of this embodiment, an expression construct comprises a pQBI25fC1 expression vector operably linked to a modified open reading frame of SEQ ID NO: 81 providing increased expression of the encoded active BoNT/E in a hamster cell line, such as, e.g., CHO and 6E6. In another aspect of this embodiment, an expression construct comprises a pQBI25fC1 expression vector operably linked to a modified open reading frame of SEQ ID NO: 82 providing increased expression of the encoded active BoNT/E in a hamster cell line, such as, e.g., CHO and 6E6.

In an aspect of this embodiment, an expression construct comprises a primate expression vector operably linked to a modified open reading frame providing increased expression of an encoded active BoNT/E in a primate cell or cell line derived from a primate cell. In an aspect of this embodiment, an expression construct comprises a pQBI25fC1 expression vector and a modified open reading frame providing increased expression of an encoded active BoNT/E in a primate cell line, such as, e.g., COS-1, COS-7 and VV-1. In another aspect of this embodiment, an expression construct comprises a pQBI25fC1 expression vector operably linked to a modified open reading frame of SEQ ID NO: 92 providing increased expression of the encoded active BoNT/E in a primate cell line, such as, e.g., COS-1, COS-7 and VV-1. In another aspect of this embodiment, an expression construct comprises a pQBI25fC1 expression vector operably linked to a modified open reading frame of SEQ ID NO: 93 providing increased expression of the encoded active BoNT/E in a primate cell line, such as, e.g., COS-1, COS-7 and VV-1. In another aspect of this embodiment, an expression construct comprises a pQBI25fC1 expression vector operably linked to a modified open reading frame of SEQ ID NO: 94 providing increased expression of the encoded active BoNT/E in a primate cell line, such as, e.g., COS-1, COS-7 and W-1.

In an aspect of this embodiment, an expression construct comprises a human expression vector operably linked to a modified open reading frame providing increased expression of an encoded active BoNT/E in a human cell or cell line derived from a human cell. In an aspect of this embodiment, an expression construct comprises a pQBI25fC1 expression vector and a modified open reading frame providing increased expression of an encoded active BoNT/E in a primate cell line, such as, e.g., SH-SY5Y, SK-N-DZ, SK-N-F1, SK-N-SH, BE (2)-C, HeLa, HEK 293, MCF-7, HepG2, HL-60, IMR-32, SW-13 and CHP3. In another aspect of this embodiment, an expression construct comprises a pQBI25fC1 expression vector operably linked to a modified open reading frame of SEQ ID NO: 95 providing increased expression of the encoded active BoNT/E in a primate cell line, such as, e.g., SH-SY5Y, SK-N-DZ, SK-N-F1, SK-N-SH, BE (2)-C, HeLa, HEK 293, MCF-7, HepG2, HL-60, IMR-32, SW-13 and CHP3. In another aspect of this embodiment, an expression construct comprises a pQBI25fC1 expression vector operably linked to a modified open reading frame of SEQ ID NO: 96 providing increased expression of the encoded active BoNT/E in a primate cell line, such as, e.g., SH-SY5Y, SK-N-DZ, SK-N-F1, SK-N-SH, BE (2)-C, HeLa, HEK 293, MCF-7, HepG2, HL-60, IMR-32, SW-13 and CHP3. In another aspect of this embodiment, an expression construct comprises a pQBI25fC1 expression vector operably linked to a modified open reading frame of SEQ ID NO: 97 providing increased expression of the encoded active BoNT/E in a primate cell line, such as, e.g., SH-SY5Y, SK-N-DZ, SK-N-F1, SK-N-SH, BE (2)-C, HeLa, HEK 293, MCF-7, HepG2, HL-60, IMR-32, SW-13 and CHP3.

Aspects of the present invention further provide cells comprising an expression construct disclosed in the present specification. It is envisioned that a cell can include, without limitation, a prokaryotic cell containing a prokaryotic expression construct useful for expressing a nucleic acid molecule comprising a modified open reading frame providing increased expression of the encoded active BoNT/E in a prokaryotic cell; a yeast cell containing a yeast expression construct useful for expressing a nucleic acid molecule comprising a modified open reading frame providing increased expression of the encoded active BoNT/E in a yeast cell; an insect cell containing an insect expression construct useful for expressing a nucleic acid molecule comprising a modified open reading frame providing increased expression of the encoded active BoNT/E in an insect cell; and a mammalian cell containing a mammalian expression construct useful for expressing a nucleic acid molecule comprising a modified open reading frame providing increased expression of the encoded active BoNT/E in a mammalian cell.

The cells disclosed in the present specification include, in part, an expression construct. In is envisioned that any and all expression constructs disclosed in the present specification can be used. Thus, aspects of this embodiment include, without limitation, cells comprising a viral expression vector operably linked to a modified open reading frame providing increased expression of an encoded active BoNT/E in a mammalian cell; a prokaryotic expression vector operably linked to a modified open reading frame providing increased expression of an encoded active BoNT/E in a prokaryotic cell; cells comprising a yeast expression vector operably linked to a modified open reading frame providing increased expression of an encoded active BoNT/E in a yeast cell; cells comprising a slime mold expression vector operably linked to a modified open reading frame providing increased expression of an encoded active BoNT/E in an slime mold cell; cells comprising a plant expression vector operably linked to a modified open reading frame providing increased expression of an encoded active BoNT/E in a plant cell or cell line derived from a plant cell; cells comprising an insect expression vector operably linked to a modified open reading frame providing increased expression of the encoded active BoNT/E in an insect cell or cell line derived from an insect cell; cells comprising a fish expression vector operably linked to a modified open reading frame providing increased expression of an encoded active BoNT/E in an fish cell or cell line derived from a fish cell; cells comprising an amphibian expression vector operably linked to a modified open reading frame providing increased expression of an encoded active BoNT/E in an amphibian cell or cell line derived from an amphibian cell; cells comprising a bird expression vector operably linked to a modified open reading frame providing increased expression of an encoded active BoNT/E in a bird cell or cell line derived from a bird cell; and cells comprising a mammalian expression vector operably linked to a modified open reading frame providing increased expression of an encoded active BoNT/E in a mammalian cell or cell line derived from a mammalian cell, such as, e.g., mouse, rat, hamster, porcine, bovine, equine, primate and human. Other aspects of this embodiment include, without limitation, expression constructs comprising a modified open reading frame that comprises any one of SEQ ID NO: 4 through SEQ ID NO: 97, SEQ ID NO: 117, SEQ ID NO: 122 or SEQ ID NO: 124.

The cells disclosed in the present specification include, in part, a heterologous cell. In is envisioned that any and all heterologous cells disclosed in the present specification can be used. Thus, aspects of this embodiment include, without limitation, prokaryotic cells prokaryotic cells including, without limitation, strains of aerobic, microaerophilic, capnophilic, facultative, anaerobic, gram-negative and gram-positive bacterial cells such as those derived from, e.g., Escherichia coli, Salmonella typhimurium, Bacillus subtilis, Bacillus licheniformis, Clostridia perfringens, Clostridia difficile, Bacteroides fragilis, Caulobacter crescentus, Methylobacterium extorquens, Lactococcus lactis and Neisseria meningirulls; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; slime mold strains, such as, e.g., those derived from, e.g., Dictyostelium discoideum; plant cells and cell lines derived from plant cells, such as, e.g., those derived from species of monocots, species of dicots, Zea mays and Arabidopsis thaliana; insect cells and cell lines derived from insects, such as, e.g., those derived from Spodoptera frugiperda, Trichoplusia ni, Drosophila melanogaster and Manduca sexta; fish cells and cell lines derived from fish cells, such as, e.g., those derived from Denio renia; amphibian cells and cell lines derived from amphibian cells, such as, e.g., those derived from Xenopus laevis and Xenopus tropicalis; bird cells and cell lines derived from bird cells, such as, e.g., those derived from Gallus gallus; mammalian cells and cell lines derived from mammalian cells, such as, e.g., those derived from mouse, rat, hamster, porcine, bovine, equine, primate and human. Cell lines may be obtained from the American Type Culture Collection (2004); European Collection of Cell Cultures (2204); and the German Collection of Microorganisms and Cell Cultures (2004). Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rae, GENERAL TECHNIQUES OF CELL CULTURE (Cambridge University Press, 1997); CELL AND TISSUE CULTURE: LABORATORY PROCEDURES (Alan Doyle et al eds., John Wiley and Sons, 1998); R. Ian Freshney, CULTURE OF ANIMAL CELLS: A MANUAL OF BASIC TECHNIQUE (Wiley-Liss, 4^{th} ed. 2000); ANIMAL CELL CULTURE: A PRACTICAL APPROACH (John R. W. Masters ed., Oxford University Press, 3^{rd} ed. 2000); MOLECULAR CLONING A LABORATORY MANUAL, supra, (2001); BASIC CELL CULTURE: A PRACTICAL APPROACH (John M. Davis, Oxford Press, 2^{nd} ed. 2002); and CURRENT PROTOCOLS IN MOLECULAR BIOLOGY, supra, (2004). These protocols are routine procedures within the scope of one skilled in the art and from the teaching herein.

It is envisioned that any and all methods for introducing an expression construct disclosed in the present specification into a cell can be used. A cell disclosed in the present specification can maintain an expression construct transiently or stably. Stably-maintained constructs may be extra-chromosomal and replicate autonomously, or they may be integrated into the chromosomal material of the cell and replicate non-autonomously. Methods useful for introducing a nucleic acid molecule into a cell including, without limitation, calcium phosphate-mediated, DEAE dextran-mediated, lipid-mediated, polybrene-mediated, polylysine-mediated, viral-mediated, microinjection, protoplast fusion, biolistic, and electroporation, see, e.g., Introducing Cloned Genes into Cultured Mammalian Cells, pp. 16.1-16.62 (Sambrook & Russell, eds., Molecular Cloning A Laboratory Manual, Vol. 3, 3^{rd} ed. 2001). One skilled in the art understands that selection of a specific method to introduce an expression construct into a cell will depend, in part, on whether the cell will transiently contain an expression construct or whether the cell will stably contain an expression construct. These protocols are routine procedures within the scope of one skilled in the art and from the teaching herein.

In an aspect of this embodiment, a chemical-mediated method, termed transfection, is used to introduce a construct expressing an active BoNT/E into a heterologous cell. In chemical-mediated methods of transfection the chemical reagent forms a complex with the nucleic acid that facilitates its uptake into the cells. Such chemical reagents include, without limitation, calcium phosphate-mediated, see, e.g., Martin Jordan & Florian Worm, Transfection of adherent and suspended cells by calcium phosphate, 33(2) Methods 136-143 (2004); diethyl-laminoethyl (DEAE) dextran-mediated, lipid-mediated, cationic polymer-mediated like polyethyleneimine (PEI)-mediated and polylysine-mediated and polybrene-mediated, see, e.g., Chun Zhang et al., Polyethylenimine strategies for plasmid delivery to brain-derived cells, 33(2) Methods 144-150 (2004). Such chemical-mediated delivery systems can be prepared by standard methods and are commercially available, see, e.g., CellPhect Transfection Kit, a DEAE-Dextran reagent, (Amersham Biosciences, Piscataway, N.J.); Mammalian Transfection Kit, Calcium phosphate and DEAE Dextran, (Stratagene, Inc., La Jolla, Calif.); LIPOFECTAMINE™ Transfection Reagent, a cationic liposome based reagent, (Invitrogen, Inc., Carlsbad, Calif.); EXGEN™ 500 Transfection kit, a 22 kDa linear polyethylenimine (PEI) reagent, (Fermentas, Inc., Hanover, Md.), and SUPERFECT®, an activated-dendrimer reagent, and EFFECTENE™, a non-liposomal lipid reagent, Transfection Kits (Qiagen, Inc., Valencia, Calif.).

In another aspect of this embodiment, a physical-mediated method is used to introduce a construct expressing an active BoNT/E into a heterologous cell. Physical reagents include, without limitation, electroporation, biolistic and microinjection. Biolistics and microinjection techniques perforate the cell wall in order to introduce the nucleic acid molecule into the cell, see, e.g., Jeike E. Biewenga et al., Plasmid-mediated gene transfer in neurons using the biolistics technique, 71(1) J. Neurosci. Methods. 67-75 (1997); and John O'Brien & Sarah C. R. Lummis, Biolistic and diolistic transfection: using the gene gun to deliver DNA and lipophilic dyes into mammalian cells, 33(2) Methods 121-125 (2004). Electroporation, also termed electropermeabilization, uses brief, high-voltage, electrical pulses to create transient pores in the membrane through which the nucleic acid molecules enter and be used effectively for stable and transient transfections of all cell types, see, e.g., M. Golzio et al., In vitro and in vivo electric field-mediated permeabilization, gene transfer, and expression, 33(2) Methods 126-135 (2004); and Oliver Greschet al., New non-viral method for gene transfer into primary cells, 33(2) Methods 151-163 (2004).

In another aspect of this embodiment, a viral-mediated method, termed transduction, is used to introduce a construct expressing an active BoNT/E into a heterologous cell. In viral-mediated methods of transient transduction, the process by which viral particles infect and replicate in a host cell has been manipulated in order to use this mechanism to introduce a nucleic acid molecule into the cell. Viral-mediated methods have been developed from a wide variety of viruses including, without limitation, retroviruses, adenoviruses, adeno-associated viruses, herpes simplex viruses, picornaviruses, alphaviruses and baculoviruses, see, e.g., Armin Blesch, Lentiviral and MLV based retroviral vectors for ex vivo and in vivo gene transfer, 33(2) Methods 164-172 (2004); and Maurizio Federico, From lentiviruses to lentivirus vectors, 229 Methods Mol. Biol. 3-15 (2003); E. M. Poeschla, Non-primate lentiviral vectors, 5(5) Curr. Opin. Mol. Ther. 529-540 (2003); Karim Benihoud et al, Adenovirus vectors for gene delivery, 10(5) Curr. Opin. Biotechnol. 440-447 (1999); H. Bueler, Adeno-associated viral vectors for gene transfer and gene therapy, 380(6) Biol. Chem. 613-622 (1999); Chooi M. Lai et al., Adenovirus and adeno-associated virus vectors, 21(12) DNA Cell Biol. 895-913 (2002); Edward A. Burton et al., Gene delivery using herpes simplex virus vectors, 21(12) DNA Cell Biol. 915-936 (2002); Paola Grandi et al., Targeting HSV amplicon vectors, 33(2) Methods 179-186 (2004); Ilya Frolov et al., Alphavirus-based expression vectors: strategies and applications, 93(21) Proc. Natl. Acad. Sci. U.S.A. 11371-11377 (1996); Markus U. Ehrengruber, Alphaviral gene transfer in neurobiology, 59(1) Brain Res. Bull. 13-22 (2002); Thomas A. Kost & J. Patrick Condreay, Recombinant baculoviruses as mammalian cell gene-delivery vectors, 20(4) Trends Biotechnol. 173-180 (2002); and A. Huser & C. Hofmann, Baculovirus vectors: novel mammalian cell gene-delivery vehicles and their applications, 3(1) Am. J. Pharmacogenomics 53-63 (2003).

Adenoviruses, which are non-enveloped, double-stranded DNA viruses, are often selected for mammalian cell transduction because adenoviruses handle relatively large nucleic acid molecules of about 36 kd, are produced at high titer, and can efficiently infect a wide variety of both dividing and non-dividing cells, see, e.g., Wim T. J. M. C. Hermens et al., Transient gene transfer to neurons and glia: analysis of adenoviral vector performance in the CNS and PNS, 71(1) J. Neurosci. Methods 85-98 (1997); and Hiroyuki Mizuguchi et al., Approaches for generating recombinant adenovirus vectors, 52(3) Adv. Drug Deliv. Rev. 165-176 (2001). Transduction using adenoviral-based system do not support prolonged protein expression because the nucleic acid molecule is carried from an episome in the cell nucleus, rather than being integrated into the host cell chromosome. Adenovirual vector systems and specific protocols for how to use such vectors are disclosed in, e.g., VIRAPOWER™ Adenoviral Expression System, a E1 and E3-deleted, pDEST-based expression vector controlled by a human cytomegalovirus (CMV) promoter, (Invitrogen, Inc., Carlsbad, Calif.) and VIRAPOWER™ Adenoviral Expression System Instruction Manual 25-0543 version A, Invitrogen, Inc., (Jul. 15, 2002); and ADEASY™ Adenoviral Vector System, an Ad5 virus-based expression vector, (Stratagene, Inc., La Jolla, Calif.) and ADEASY™ Adenoviral Vector System Instruction Manual 064004f, Stratagene, Inc.

Nucleic acid molecule delivery can also use single-stranded RNA retroviruses viruses, such as, e.g., oncoretroviruses and lentiviruses. Retroviral-mediated transduction often produce transduction efficiencies close to 100%, can easily control the proviral copy number by varying the multiplicity of infection (MOI), and can be used to either transiently or stably transduce cells, see, e.g., Tiziana Tonini et al., Transient production of retroviral- and lentiviral-based vectors for the transduction of Mammalian cells, 285 Methods Mol. Biol. 141-148 (2004); Armin Blesch, Lentiviral and MLV based retroviral vectors for ex vivo and in vivo gene transfer, 33(2) Methods 164-172 (2004); Félix Recillas-Targa, Gene transfer and expression in mammalian cell lines and transgenic animals, 267 Methods Mol. Biol. 417-433 (2004); and Roland Wolkowicz et al., Lentiviral vectors for the delivery of DNA into mammalian cells, 246 Methods Mol. Biol. 391-411 (2004). Retroviral particles consist of an RNA genome packaged in a protein capsid, surrounded by a lipid envelope. The retrovirus infects a host cell by injecting its RNA into the cytoplasm along with the reverse transcriptase enzyme. The RNA template is then reverse transcribed into a linear, double stranded cDNA that replicates itself by integrating into the host cell genome. Viral particles are spread both vertically (from parent cell to daughter cells via the provirus) as well as horizontally (from cell to cell via virions). This replication strategy enables long-term persist expression since the nucleic acid molecules of interest are stably integrated into a chromosome of the host cell, thereby enabling long-term expression of the protein. For instance, animal studies have shown that lentiviral vectors injected into a variety of tissues produced sustained protein expression for more than 1 year, see, e.g., Luigi Naldini et al., In vivo gene delivery and stable transduction of non-dividing cells by a lentiviral vector, 272(5259) Science 263-267 (1996). The Oncoretroviruses-derived vector systems, such as, e.g., Moloney murine leukemia virus (MoMLV), are widely used and infect many different non-dividing cells. Lentiviruses can also infect many different cell types, including dividing and non-dividing cells and possess complex envelope proteins, which allows for highly specific cellular targeting.

Retroviral vectors and specific protocols for how to use such vectors are disclosed in, e.g., U.S. Patent Nos. Manfred Gossen & Hermann Bujard, Tight control of gene expression in eukaryotic cells by tetracycline-responsive promoters, U.S. Pat. No. 5,464,758 (Nov. 7, 1995) and Hermann Bujard & Manfred Gossen, Methods for regulating gene expression, U.S. Pat. No. 5,814,618 (Sep. 29, 1998) David S. Hogness, Polynucleotides encoding insect steroid hormone receptor polypeptides and cells transformed with same, U.S. Pat. No. 5,514,578 (May 7, 1996) and David S. Hogness, Polynucleotide encoding insect ecdysone receptor, U.S. Pat. No. 6,245,531 (Jun. 12, 2001); Elisabetta Vegeto et al., Progesterone receptor having C. terminal hormone binding domain truncations, U.S. Pat. No. 5,364,791 (Nov. 15, 1994), Elisabetta Vegeto et al., Mutated steroid hormone receptors, methods for their use and molecular switch for gene therapy, U.S. Pat. No. 5,874,534 (Feb. 23, 1999) and Elisabetta Vegeto et al., Mutated steroid hormone receptors, methods for their use and molecular switch for gene therapy, U.S. Pat. No. 5,935,934 (Aug. 10, 1999). Furthermore, such viral delivery systems can be prepared by standard methods and are commercially available, see, e.g., BD™ Tet-Off and Tet-On Gene Expression Systems, a tetracycline-inducible mammalian adenovirus-based expression vector, (BD Biosciences-Clonetech, Palo Alto, Calif.) and BD™ Tet-Off and Tet-On Gene Expression Systems User Manual, PT3001-1, BD Biosciences Clonetech, (Mar. 14, 2003), GENESWITCH™ System, an inducible mammalian adenovirus-based expression vector, (Invitrogen, Inc., Carlsbad, Calif.) and GENESWITCH™ System A Mifepristone-Regulated Expression System for Mammalian Cells version D, 25-0313, Invitrogen, Inc., (Nov. 4, 2002); VIRAPOWER™ Lentiviral Expression System, a replication-incomplete, HIV-1-based lentivirus expression vector, (Invitrogen, Inc., Carlsbad, Calif.) and VIRAPOWER™ Lentiviral Expression System Instruction Manual 25-0501 version E, Invitrogen, Inc., (Dec. 8, 2003); and COMPLETE CONTROL® Retroviral Inducible Mammalian Expression System, an ecdysone-inducible lentivirus-based mammalian expression vector, (Stratagene, La Jolla, Calif.) and Complete Control® COMPLETE CONTROL® Retroviral Inducible Mammalian Expression System Instruction Manual, 064005e.

Thus, in an embodiment, a cell comprises a mammalian cell comprising an expression construct operably linked to a nucleic acid molecule comprising a modified open reading frame providing increased expression of the encoded active BoNT/E in a mammalian cell. In an aspect of this embodiment, a cell comprises a mammalian cell transiently containing an expression construct operably linked to a nucleic acid molecule comprising a modified open reading frame providing increased expression of the encoded active BoNT/E in a mammalian cell. In another aspect of this embodiment, a cell comprises a mammalian cell stably containing an expression construct operably linked to a nucleic acid molecule comprising a modified open reading frame providing increased expression of the encoded active BoNT/E in a mammalian cell. In yet another aspect of this embodiment, an expression construct is a viral expression construct. In further aspect of this embodiment, a viral expression construct is a lentivirus expression construct, a fowl pox virus expression construct, a pseudorabies virus expression construct, a retrovirus expression construct, a semliki forest virus expression construct, a sindbis virus expression construct, a vaccinia virus expression construct, or an adenovirus expression construct. In yet other aspect of this embodiment, a nucleic acid molecule comprises any of the modified open reading frames of SEQ ID NO: 56 through SEQ ID NO: 97.

Thus, in an embodiment, a cell comprises a prokaryotic cell comprising an expression construct operably linked to a nucleic acid molecule comprising a modified open reading frame providing increased expression of the encoded active BoNT/E in a prokaryotic cell. In an aspect of this embodiment, a cell comprises a prokaryotic cell transiently containing an expression construct operably linked to a nucleic acid molecule comprising a modified open reading frame providing increased expression of the encoded active BoNT/E in a prokaryotic cell. In another aspect of this embodiment, a cell comprises a prokaryotic cell stably containing an expression construct operably linked to a nucleic acid molecule comprising a modified open reading frame providing increased expression of the encoded active BoNT/E in a prokaryotic cell. In a further aspect of this embodiment, a prokaryotic cell is derived from an aerobic bacterium, a microaerophilic bacterium, a capnophilic bacterium, a facultative bacterium, an anaerobic bacterium, a gram-negative bacterium or a gram-positive bacterium. In a further aspect of this embodiment, a prokaryotic cell is a prokaryotic strain derived from Escherichia coli, Salmonella typhimurium, Bacillus subtilis, Bacillus licheniformis, Clostridia perfringens, Clostridia difficile, Bacteroides fragilis, Caulobacter crescentus, Methylobacterium extorquens, Lactococcus lactis or Neisseria meningirulls. In yet another aspect of this embodiment, an expression construct is a prokaryotic expression construct. In yet another aspect of this embodiment, a nucleic acid molecule comprises any of the modified open reading frames of SEQ ID NO: 4 through SEQ ID NO: 34, SEQ ID NO: 117, SEQ ID NO: 122 and SEQ ID NO: 124.

In an embodiment, a cell comprises an eukaryotic cell comprising an expression construct operably linked to a nucleic acid molecule comprising a modified open reading frame providing increased expression of the encoded active BoNT/E in an eukaryotic cell. In an aspect of this embodiment, a cell comprises an eukaryotic cell transiently containing an expression construct operably linked to a nucleic acid molecule comprising a modified open reading frame providing increased expression of the encoded active BoNT/E in an eukaryotic cell. In another aspect of this embodiment, a cell comprises an eukaryotic cell stably containing an expression construct operably linked to a nucleic acid molecule comprising a modified open reading frame providing increased expression of the encoded active BoNT/E in an eukaryotic cell. In yet another aspect of this embodiment, an expression construct is an eukaryotic expression construct. In yet other aspect of this embodiment, a nucleic acid molecule comprises any of the modified open reading frames of SEQ ID NO: 35 through SEQ ID NO: 97.

In an embodiment, a cell comprises a yeast cell comprising an expression construct operably linked to a nucleic acid molecule comprising a modified open reading frame providing increased expression of the encoded active BoNT/E in a yeast cell. In an aspect of this embodiment, a cell comprises a yeast cell transiently containing an expression construct operably linked to a nucleic acid molecule comprising a modified open reading frame providing increased expression of the encoded active BoNT/E in a yeast cell. In another aspect of this embodiment, a cell comprises a yeast cell stably containing an expression construct operably linked to a nucleic acid molecule comprising a modified open reading frame providing increased expression of the encoded active BoNT/E in a yeast cell. In a further aspect of this embodiment, a yeast cell is a yeast strain derived from Pichia pastoris, Pichia methanolica, Pichia angusta, Schizosaccharomyces pombe, Saccharomyces cerevisiae or Yarrowia lipolytica. In yet another aspect of this embodiment, an expression construct is a yeast expression construct. In yet another aspect of this embodiment, a nucleic acid molecule comprises any of the modified open reading frames of SEQ ID NO: 35 through SEQ ID NO: 46.

In an embodiment, a cell comprises a slime mold cell comprising an expression construct operably linked to a nucleic acid molecule comprising a modified open reading frame providing increased expression of the encoded active BoNT/E in a slime mold cell. In an aspect of this embodiment, a cell comprises a slime mold cell transiently containing an expression construct operably linked to a nucleic acid molecule comprising a modified open reading frame providing increased expression of the encoded active BoNT/E in a slime mold cell. In another aspect of this embodiment, a cell comprises a slime mold cell stably containing an expression construct operably linked to a nucleic acid molecule comprising a modified open reading frame providing increased expression of the encoded active BoNT/E in a slime mold cell. In a further aspect of this embodiment, a slime mold cell is a slime mold strain derived from Dictyostelium discoideum. In yet another aspect of this embodiment, an expression construct is a slime mold expression construct. In yet another aspect of this embodiment, a nucleic acid molecule comprises any of the modified open reading frames of SEQ ID NO: 47 through SEQ ID NO: 49.

In an embodiment, a cell comprises a plant cell comprising an expression construct operably linked to a nucleic acid molecule comprising a modified open reading frame providing increased expression of the encoded active BoNT/E in a plant cell. In an aspect of this embodiment, a cell comprises a plant cell transiently containing an expression construct operably linked to a nucleic acid molecule comprising a modified open reading frame providing increased expression of the encoded active BoNT/E in a plant cell. In another aspect of this embodiment, a cell comprises a plant cell stably containing an expression construct operably linked to a nucleic acid molecule comprising a modified open reading frame providing increased expression of the encoded active BoNT/E in a plant cell. In a further aspect of this embodiment, a plant cell is derived from a monocot cell or cell line derived from a monocot cell or a dicot cell or cell line derived from a dicot cell. In a further aspect of this embodiment, a plant cell or cell line derived from a plant cell is from Zea mays or Arabidopsis thaliana. In yet another aspect of this embodiment, an expression construct is a plant expression construct. In yet another aspect of this embodiment, a nucleic acid molecule comprises any of the modified open reading frames of SEQ ID NO: 50 through SEQ ID NO: 55.

In an embodiment, a cell comprises an insect cell comprising an expression construct operably linked to a nucleic acid molecule comprising a modified open reading frame providing increased expression of the encoded active BoNT/E in an insect cell. In an aspect of this embodiment, a cell comprises an insect cell transiently containing an expression construct operably linked to a nucleic acid molecule comprising a modified open reading frame providing increased expression of the encoded active BoNT/E in an insect cell. In another aspect of this embodiment, a cell comprises an insect cell stably containing an expression construct operably linked to a nucleic acid molecule comprising a modified open reading frame providing increased expression of the encoded active BoNT/E in an insect cell. In a further aspect of this embodiment, an insect cell is an insect strain derived from Spodoptera frugiperda, Trichoplusia ni, Drosophila melanogaster or Manduca sexta. In a further aspect of this embodiment, an insect cell is an insect cell line derived from Sf9, Sf21, High-five, S2 and Kc. In yet another aspect of this embodiment, an expression construct is an insect expression construct. In yet another aspect of this embodiment, a nucleic acid molecule In yet another aspect of this embodiment, a nucleic acid molecule comprises any of the modified open reading frames of SEQ ID NO: 56 through SEQ ID NO: 61. In additional aspects of this embodiment, a Sf9 cell line contains an expression construct operably linked to a nucleic acid molecule comprising a modified open reading frame of SEQ ID NO: 59, SEQ ID NO: 60 or SEQ ID NO: 61; a Sf21 cell line contains an expression construct operably linked to a nucleic acid molecule comprising a modified open reading frame of SEQ ID NO: 59, SEQ ID NO: 60 or SEQ ID NO: 61; a High-Five cell line contains an expression construct operably linked to a nucleic acid molecule comprising a modified open reading frame of SEQ ID NO: 59, SEQ ID NO: 60 or SEQ ID NO: 61; a S2 cell line contains an expression construct operably linked to a nucleic acid molecule comprising a modified open reading frame of SEQ ID NO: 56, SEQ ID NO: 57 or SEQ ID NO: 58; or a Kc cell line contains an expression construct operably linked to a nucleic acid molecule comprising a modified open reading frame of SEQ ID NO: 56, SEQ ID NO: 57 or SEQ ID NO: 58.

In an embodiment, a cell comprises a fish cell comprising an expression construct operably linked to a nucleic acid molecule comprising a modified open reading frame providing increased expression of the encoded active BoNT/E in a fish cell. In an aspect of this embodiment, a cell comprises a fish cell transiently containing an expression construct operably linked to a nucleic acid molecule comprising a modified open reading frame providing increased expression of the encoded active BoNT/E in a fish cell. In another aspect of this embodiment, a cell comprises a fish cell stably containing an expression construct operably linked to a nucleic acid molecule comprising a modified open reading frame providing increased expression of the encoded active BoNT/E in a fish cell. In a further aspect of this embodiment, a fish cell is a fish cell or cell line derived from a fish cell from Denio renia. In yet another aspect of this embodiment, an expression construct is a fish expression construct. In yet another aspect of this embodiment, a nucleic acid molecule comprises any of the modified open reading frames of SEQ ID NO: 62 through SEQ ID NO: 64.

In an embodiment, a cell comprises an amphibian cell comprising an expression construct operably linked to a nucleic acid molecule comprising a modified open reading frame providing increased expression of the encoded active BoNT/E in an amphibian cell. In an aspect of this embodiment, a cell comprises an amphibian cell transiently containing an expression construct operably linked to a nucleic acid molecule comprising a modified open reading frame providing increased expression of the encoded active BoNT/E in an amphibian cell. In another aspect of this embodiment, a cell comprises an amphibian cell stably containing an expression construct operably linked to a nucleic acid molecule comprising a modified open reading frame providing increased expression of the encoded active BoNT/E in an amphibian cell. In a further aspect of this embodiment, an amphibian cell is an amphibian cell or cell line derived from an amphibian cell from Xenopus laevis. In a further aspect of this embodiment, an amphibian cell is an amphibian cell or cell line derived from an amphibian cell from Xenopus tropicalis. In yet another aspect of this embodiment, an expression construct is an amphibian expression construct. In yet another aspect of this embodiment, a nucleic acid molecule comprises any of the modified open reading frames of SEQ ID NO: 65 through SEQ ID NO: 70.

In an embodiment, a cell comprises a bird cell comprising an expression construct operably linked to a nucleic acid molecule comprising a modified open reading frame providing increased expression of the encoded active BoNT/E in a bird cell. In an aspect of this embodiment, a cell comprises a bird cell transiently containing an expression construct operably linked to a nucleic acid molecule comprising a modified open reading frame providing increased expression of the encoded active BoNT/E in a bird cell. In another aspect of this embodiment, a cell comprises a bird cell stably containing an expression construct operably linked to a nucleic acid molecule comprising a modified open reading frame providing increased expression of the encoded active BoNT/E in a bird cell. In a further aspect of this embodiment, a bird cell is a bird cell or cell line derived from a bird cell from Gallus gallus. In yet another aspect of this embodiment, an expression construct is a bird expression construct. In yet another aspect of this embodiment, a nucleic acid molecule comprises any of the modified open reading frames of SEQ ID NO: 71 through SEQ ID NO: 73.

In an embodiment, a cell comprises a mammalian cell comprising an expression construct operably linked to a nucleic acid molecule comprising a modified open reading frame providing increased expression of the encoded active BoNT/E in a mammalian cell. In an aspect of this embodiment, a cell comprises a mammalian cell transiently containing an expression construct operably linked to a nucleic acid molecule comprising a modified open reading frame providing increased expression of the encoded active BoNT/E in a mammalian. In another aspect of this embodiment, a cell comprises a mammalian cell stably containing an expression construct operably linked to a nucleic acid molecule comprising a modified open reading frame providing increased expression of the encoded active BoNT/E in a mammalian cell. In a further aspect of this embodiment, a mammalian cell is a mammalian cell or cell line derived from a mammalian cell from a mouse, a rat, a hamster, a porcine, a bovine, an equine, a primate or a human. In yet another aspect of this embodiment, an expression construct is a mammalian expression construct. In yet another aspect of this embodiment, a nucleic acid molecule comprises any of the modified open reading frames of SEQ ID NO: 74 through SEQ ID NO: 97.

In an embodiment, a cell comprises a mouse cell comprising an expression construct operably linked to a nucleic acid molecule comprising a modified open reading frame providing increased expression of the encoded active BoNT/E in a mouse cell. In an aspect of this embodiment, a cell comprises a mouse cell transiently containing an expression construct operably linked to a nucleic acid molecule comprising a modified open reading frame providing increased expression of the encoded active BoNT/E in a mouse cell. In another aspect of this embodiment, a cell comprises a mouse cell stably containing an expression construct operably linked to a nucleic acid molecule comprising a modified open reading frame providing increased expression of the encoded active BoNT/E in a mouse cell. In further aspect of this embodiment, a mouse cell is a mouse cell or cell line derived from a mouse cell from M. musculus. In a further aspect of this embodiment, a mouse cell is a mouse cell line derived from 10T1/2, BALB/3T3, L-M, NB4 1A3, NIE-115, NG108-15, NIH3T3, NCTC or Neuro 2A. In yet another aspect of this embodiment, an expression construct is a mouse expression construct. In yet another aspect of this embodiment, a nucleic acid molecule comprises any of the modified open reading frames of SEQ ID NO: 74 through SEQ ID NO: 76. In additional aspects of this embodiment, a Neuro 2A cell line contains an expression construct operably linked to a nucleic acid molecule comprising a modified open reading frame of SEQ ID NO: 74, SEQ ID NO: 75 or SEQ ID NO: 76; a 10T1/2 cell line contains an expression construct operably linked to a nucleic acid molecule comprising a modified open reading frame of SEQ ID NO: 74, SEQ ID NO: 75 or SEQ ID NO: 76; a BALB/3T3 cell line contains an expression construct operably linked to a nucleic acid molecule comprising a modified open reading frame of SEQ ID NO: 74, SEQ ID NO: 75 or SEQ ID NO: 76; a NG108-15 cell line contains an expression construct operably linked to a nucleic acid molecule comprising a modified open reading frame of SEQ ID NO: 74, SEQ ID NO: 75 or SEQ ID NO: 76; or a NIE-115 cell line contains an expression construct operably linked to a nucleic acid molecule comprising a modified open reading frame of SEQ ID NO: 74, SEQ ID NO: 75 or SEQ ID NO: 76.

In an embodiment, a cell comprises a rat cell comprising an expression construct operably linked to a nucleic acid molecule comprising a modified open reading frame providing increased expression of the encoded active BoNT/E in a rat cell. In an aspect of this embodiment, a cell comprises a rat cell transiently containing an expression construct operably linked to a nucleic acid molecule comprising a modified open reading frame providing increased expression of the encoded active BoNT/E in a rat cell. In another aspect of this embodiment, a cell comprises a rat cell stably containing an expression construct operably linked to a nucleic acid molecule comprising a modified open reading frame providing increased expression of the encoded active BoNT/E in a rat cell. In further aspect of this embodiment, a rat cell is a rat cell or cell line derived from a rat cell from R. norvegicus. In a further aspect of this embodiment, a rat cell is a rat cell line derived from PC12, GH1, GH3, C6 or L2. In yet another aspect of this embodiment, an expression construct is a rat expression construct. In yet another aspect of this embodiment, a nucleic acid molecule comprises any of the modified open reading frames of SEQ ID NO: 77 through SEQ ID NO: 79. In additional aspects of this embodiment, a PC12 cell line contains an expression construct operably linked to a nucleic acid molecule comprising a modified open reading frame of SEQ ID NO: 77, SEQ ID NO: 78 or SEQ ID NO: 79; a GH1 cell line contains an expression construct operably linked to a nucleic acid molecule comprising a modified open reading frame of SEQ ID NO: 77, SEQ ID NO: 78 or SEQ ID NO: 79; a GH3 cell line contains an expression construct operably linked to a nucleic acid molecule comprising a modified open reading frame of SEQ ID NO: 77, SEQ ID NO: 78 or SEQ ID NO: 79; a C6 cell line contains an expression construct operably linked to a nucleic acid molecule comprising a modified open reading frame of SEQ ID NO: 77, SEQ ID NO: 78 or SEQ ID NO: 79; or a L2 cell line contains an expression construct operably linked to a nucleic acid molecule comprising a modified open reading frame of SEQ ID NO: 77, SEQ ID NO: 78 or SEQ ID NO: 79.

In an embodiment, a cell comprises a hamster cell comprising an expression construct operably linked to a nucleic acid molecule comprising a modified open reading frame providing increased expression of the encoded active BoNT/E in a hamster cell. In an aspect of this embodiment, a cell comprises a hamster cell transiently containing an expression construct operably linked to a nucleic acid molecule comprising a modified open reading frame providing increased expression of the encoded active BoNT/E in a hamster cell. In another aspect of this embodiment, a cell comprises a hamster cell stably containing an expression construct operably linked to a nucleic acid molecule comprising a modified open reading frame providing increased expression of the encoded active BoNT/E in a hamster cell. In further aspect of this embodiment, a hamster cell is a hamster cell or cell line derived from a hamster cell from C. griseus. In a further aspect of this embodiment, a hamster cell is a hamster cell line derived from CHO or 6E6. In yet another aspect of this embodiment, an expression construct is a hamster expression construct. In yet another aspect of this embodiment, a nucleic acid molecule comprises any of the modified open reading frames of SEQ ID NO: 80 through SEQ ID NO: 81. In additional aspects of this embodiment, a CHO cell line contains an expression construct operably linked to a nucleic acid molecule comprising a modified open reading frame of SEQ ID NO: 80, SEQ ID NO: 81 or SEQ ID NO: 82; or a 6E6 cell line contains an expression construct operably linked to a nucleic acid molecule comprising a modified open reading frame of SEQ ID NO: 80, SEQ ID NO: 81 or SEQ ID NO: 82.

In an embodiment, a cell comprises a porcine cell comprising an expression construct operably linked to a nucleic acid molecule comprising a modified open reading frame providing increased expression of the encoded active BoNT/E in a porcine cell. In an aspect of this embodiment, a cell comprises a porcine cell transiently containing an expression construct operably linked to a nucleic acid molecule comprising a modified open reading frame providing increased expression of the encoded active BoNT/E in a porcine cell. In another aspect of this embodiment, a cell comprises a porcine cell stably containing an expression construct operably linked to a nucleic acid molecule comprising a modified open reading frame providing increased expression of the encoded active BoNT/E in a porcine cell. In further aspect of this embodiment, a porcine cell is a porcine cell or cell line derived from a porcine cell from S. scrofa. In a further aspect of this embodiment, a porcine cell is a porcine cell line derived from PK15, LLC-PK1, ST or ESK-4. In yet another aspect of this embodiment, an expression construct is a porcine expression construct. In yet another aspect of this embodiment, a nucleic acid molecule comprises any of the modified open reading frames of SEQ ID NO: 83 through SEQ ID NO: 85.

In an embodiment, a cell comprises a bovine cell comprising an expression construct operably linked to a nucleic acid molecule comprising a modified open reading frame providing increased expression of the encoded active BoNT/E in a bovine cell. In an aspect of this embodiment, a cell comprises a bovine cell transiently containing an expression construct operably linked to a nucleic acid molecule comprising a modified open reading frame providing increased expression of the encoded active BoNT/E in a bovine cell. In another aspect of this embodiment, a cell comprises a bovine cell stably containing an expression construct operably linked to a nucleic acid molecule comprising a modified open reading frame providing increased expression of the encoded active BoNT/E in a bovine cell. In further aspect of this embodiment, a bovine cell is a bovine cell or cell line derived from a bovine cell from B. taurus. In a further aspect of this embodiment, a bovine cell is a bovine cell line derived from CPAE, BT, SBAC or FB2. In yet another aspect of this embodiment, an expression construct is a bovine expression construct. In yet another aspect of this embodiment, a nucleic acid molecule comprises any of the modified open reading frames of SEQ ID NO: 86 through SEQ ID NO: 88.

In an embodiment, a cell comprises an equine cell comprising an expression construct operably linked to a nucleic acid molecule comprising a modified open reading frame providing increased expression of the encoded active BoNT/E in an equine cell. In an aspect of this embodiment, a cell comprises an equine cell transiently containing an expression construct operably linked to a nucleic acid molecule comprising a modified open reading frame providing increased expression of the encoded active BoNT/E in an equine cell. In another aspect of this embodiment, a cell comprises an equine cell stably containing an expression construct operably linked to a nucleic acid molecule comprising a modified open reading frame providing increased expression of the encoded active BoNT/E in an equine cell. In further aspect of this embodiment, an equine cell is an equine cell or cell line derived from an equine cell from E. caballus. In a further aspect of this embodiment, an equine cell is an equine cell line derived from NBL-6. In yet another aspect of this embodiment, an expression construct is an equine expression construct. In yet another aspect of this embodiment, a nucleic acid molecule comprises any of the modified open reading frames of SEQ ID NO: 89 through SEQ ID NO: 92.

In an embodiment, a cell comprises a primate cell comprising an expression construct operably linked to a nucleic acid molecule comprising a modified open reading frame providing increased expression of the encoded active BoNT/E in a primate cell. In an aspect of this embodiment, a cell comprises a primate cell transiently containing an expression construct operably linked to a nucleic acid molecule comprising a modified open reading frame providing increased expression of the encoded active BoNT/E in a primate cell. In another aspect of this embodiment, a cell comprises a primate cell stably containing an expression construct operably linked to a nucleic acid molecule comprising a modified open reading frame providing increased expression of the encoded active BoNT/E in a primate cell. In further aspect of this embodiment, a primate cell is a primate cell or cell line derived from a primate cell from C. aethiops. In a further aspect of this embodiment, a primate cell is a primate cell line derived from COS-1, COS-7 or VV-1. In yet another aspect of this embodiment, a nucleic acid molecule comprises any of the modified open reading frames of SEQ ID NO: 92 through SEQ ID NO: 94. In additional aspects of this embodiment, a COS-1 cell line contains an expression construct operably linked to a nucleic acid molecule comprising a modified open reading frame of SEQ ID NO: 92, SEQ ID NO: 93 or SEQ ID NO: 94; a COS-7 cell line contains an expression construct operably linked to a nucleic acid molecule comprising a modified open reading frame of SEQ ID NO: 92, SEQ ID NO: 93 or SEQ ID NO: 94; or a VV-1 cell line contains an expression construct operably linked to a nucleic acid molecule comprising a modified open reading frame of SEQ ID NO: 92, SEQ ID NO: 93 or SEQ ID NO: 94.

In an embodiment, a cell comprises a human cell comprising an expression construct operably linked to a nucleic acid molecule comprising a modified open reading frame providing increased expression of the encoded active BoNT/E in a human cell. In an aspect of this embodiment, a cell comprises a human cell transiently containing an expression construct operably linked to a nucleic acid molecule comprising a modified open reading frame providing increased expression of the encoded active BoNT/E in a human cell. In another aspect of this embodiment, a cell comprises a human cell stably containing an expression construct operably linked to a nucleic acid molecule comprising a modified open reading frame providing increased expression of the encoded active BoNT/E in a human cell. In further aspect of this embodiment, a human cell is a human cell or cell line derived from a human cell from H. sapiens. In a further aspect of this embodiment, a human cell is a human cell line derived from SH-SY5Y, SK-N-DZ, SK-N-F1, SK-N-SH, BE (2)-C, HeLa, HEK 293, MCF-7, HepG2, HL-60, IMR-32, SW-13 or CHP3. In yet another aspect of this embodiment, an expression construct is a human expression construct. In yet another aspect of this embodiment, a nucleic acid molecule comprises any of the modified open reading frames of SEQ ID NO: 95 through SEQ ID NO: 97. In additional aspects of this embodiment, a SH-SY5Y cell line contains an expression construct operably linked to a nucleic acid molecule comprising a modified open reading frame of SEQ ID NO: 95, SEQ ID NO: 96 or SEQ ID NO: 97; a SK-N-DZ cell line contains an expression construct operably linked to a nucleic acid molecule comprising a modified open reading frame of SEQ ID NO: 95, SEQ ID NO: 96 or SEQ ID NO: 97; a SK-N-F1 cell line contains an expression construct operably linked to a nucleic acid molecule comprising a modified open reading frame of SEQ ID NO: 95, SEQ ID NO: 96 or SEQ ID NO: 97; a SK-N-SH cell line contains an expression construct operably linked to a nucleic acid molecule comprising a modified open reading frame of SEQ ID NO: 95, SEQ ID NO: 96 or SEQ ID NO: 97; a BE (2)-C cell line contains an expression construct operably linked to a nucleic acid molecule comprising a modified open reading frame of SEQ ID NO: 95, SEQ ID NO: 96 or SEQ ID NO: 97; a HeLa cell line contains an expression construct operably linked to a nucleic acid molecule comprising a modified open reading frame of SEQ ID NO: 95, SEQ ID NO: 96 or SEQ ID NO: 97; a HEK 293 cell line contains an expression construct operably linked to a nucleic acid molecule comprising a modified open reading frame of SEQ ID NO: 95, SEQ ID NO: 96 or SEQ ID NO: 97; a MCF-7 cell line contains an expression construct operably linked to a nucleic acid molecule comprising a modified open reading frame of SEQ ID NO: 95, SEQ ID NO: 96 or SEQ ID NO: 97; a HepG2 cell line contains an expression construct operably linked to a nucleic acid molecule comprising a modified open reading frame of SEQ ID NO: 95, SEQ ID NO: 96 or SEQ ID NO: 97; a HepG2 cell line contains an expression construct operably linked to a nucleic acid molecule comprising a modified open reading frame of SEQ ID NO: 95, SEQ ID NO: 96 or SEQ ID NO: 97; a HL-60 cell line contains an expression construct operably linked to a nucleic acid molecule comprising a modified open reading frame of SEQ ID NO: 95, SEQ ID NO: 96 or SEQ ID NO: 97; a IMR-32 cell line contains an expression construct operably linked to a nucleic acid molecule comprising a modified open reading frame of SEQ ID NO: 95, SEQ ID NO: 96 or SEQ ID NO: 97; a SW-13 cell line contains an expression construct operably linked to a nucleic acid molecule comprising a modified open reading frame of SEQ ID NO: 95, SEQ ID NO: 96 or SEQ ID NO: 97; or a CHP3 cell line contains an expression construct operably linked to a nucleic acid molecule comprising a modified open reading frame of SEQ ID NO: 95, SEQ ID NO: 96 or SEQ ID NO: 97.

Another aspect of the present invention provides a method of producing an active BoNT/E comprising the step of expressing an expression construct comprising a modified open reading frame providing increased expression of an encoded active BoNT/E in a heterologous cell. In another aspect of the present invention provides a method of producing an active BoNT/E comprising the steps of introducing an expression construct comprising a modified open reading frame providing increased expression of an encoded active BoNT/E into a heterologous cell and expressing the expression construct in the heterologous cell.

The methods disclosed in the present specification include, in part, an active BoNT/E. In is envisioned that any and all active BoNT/E disclosed in the present specification can be produced using the methods disclosed in the present specification. Thus, aspects of this embodiment include producing, without limitation, active BoNT/E, naturally occurring active BoNT/E variants, such as, e.g., BoNT/E isoforms, non-naturally occurring active BoNT/E variants, such as, e.g., conservative BoNT/E variants, non-conservative BoNT/E variants and active BoNT/E fragments thereof, or any combination thereof. Other aspects of this embodiment include, without limitation, active BoNT/E of SEQ ID NO:1, naturally occurring active BoNT/E variants of SEQ ID NO: 1, such as, e.g., active BoNT/E isoforms of SEQ ID NO: 1, non-naturally occurring active BoNT/E variants of SEQ ID NO: 1, such as, e.g., conservative BoNT/E variants of SEQ ID NO: 1, non-conservative BoNT/E variants of SEQ ID NO: 1 and active BoNT/E fragments of SEQ ID NO: 1, or any combination thereof.

The methods disclosed in the present specification include, in part, an expression construct. In is envisioned that any and all expression constructs disclosed in the present specification can be used. Thus, aspects of this embodiment include, without limitation, cells comprising a viral expression vector operably linked to a modified open reading frame providing increased expression of an encoded active BoNT/E in a mammalian cell; a prokaryotic expression vector operably linked to a modified open reading frame providing increased expression of an encoded active BoNT/E in a prokaryotic cell; cells comprising a yeast expression vector operably linked to a modified open reading frame providing increased expression of an encoded active BoNT/E in a yeast cell; cells comprising a slime mold expression vector operably linked to a modified open reading frame providing increased expression of an encoded active BoNT/E in an slime mold cell; cells comprising a plant expression vector operably linked to a modified open reading frame providing increased expression of an encoded active BoNT/E in a plant cell or cell line derived from a plant cell; cells comprising an insect expression vector operably linked to a modified open reading frame providing increased expression of the encoded active BoNT/E in an insect cell or cell line derived from an insect cell; cells comprising a fish expression vector operably linked to a modified open reading frame providing increased expression of an encoded active BoNT/E in an fish cell or cell line derived from a fish cell; cells comprising an amphibian expression vector operably linked to a modified open reading frame providing increased expression of an encoded active BoNT/E in an amphibian cell or cell line derived from an amphibian cell; cells comprising a bird expression vector operably linked to a modified open reading frame providing increased expression of an encoded active BoNT/E in a bird cell or cell line derived from a bird cell; and cells comprising a mammalian expression vector operably linked to a modified open reading frame providing increased expression of an encoded active BoNT/E in a mammalian cell or cell line derived from a mammalian cell, such as, e.g., mouse, rat, hamster, porcine, bovine, equine, primate and human. Other aspects of this embodiment include, without limitation, expression constructs comprising a modified open reading frame that comprises any one of SEQ ID NO: 4 through SEQ ID NO: 97, SEQ ID NO: 117, SEQ ID NO: 122 or SEQ ID NO: 124.

The methods disclosed in the present specification include, in part, a heterologous cell. In is envisioned that any and all heterologous cells disclosed in the present specification can be used. Thus, aspects of this embodiment include, without limitation, prokaryotic cells prokaryotic cells including, without limitation, strains of aerobic, microaerophilic, capnophilic, facultative, anaerobic, gram-negative and gram-positive bacterial cells such as those derived from, e.g., Escherichia coli, Salmonella typhimurium, Bacillus subtilis, Bacillus licheniformis, Clostridia perfringens, Clostridia difficile, Bacteroides fragilis, Caulobacter crescentus, Methylobacterium extorquens, Lactococcus lactis and Neisseria meningirulls; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; slime mold strains, such as, e.g., those derived from, e.g., Dictyostelium discoideum; plant cells and cell lines derived from plant cells, such as, e.g., those derived from species of monocots, species of dicots, Zea mays and Arabidopsis thaliana; insect cells and cell lines derived from insects, such as, e.g., those derived from Spodoptera frugiperda, Trichoplusia ni, Drosophila melanogaster and Manduca sexta; fish cells and cell lines derived from fish cells, such as, e.g., those derived from Denio renia; amphibian cells and cell lines derived from amphibian cells, such as, e.g., those derived from Xenopus laevis and Xenopus tropicalis; bird cells and cell lines derived from bird cells, such as, e.g., those derived from Gallus gallus; mammalian cells and cell lines derived from mammalian cells, such as, e.g., those derived from mouse, rat, hamster, porcine, bovine, equine, primate and human. Cell lines may be obtained from the American Type Culture Collection (2004); European Collection of Cell Cultures (2204); and the German Collection of Microorganisms and Cell Cultures (2004). Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rae, GENERAL TECHNIQUES OF CELL CULTURE (Cambridge University Press, 1997); CELL AND TISSUE CULTURE: LABORATORY PROCEDURES (Alan Doyle et al eds., John Wiley and Sons, 1998); R. Ian Freshney, CULTURE OF ANIMAL CELLS: A MANUAL OF BASIC TECHNIQUE (Wiley-Liss, 4^{th} ed. 2000); ANIMAL CELL CULTURE: A PRACTICAL APPROACH (John R. W. Masters ed., Oxford University Press, 3^{rd} ed. 2000); MOLECULAR CLONING A LABORATORY MANUAL, supra, (2001); BASIC CELL CULTURE: A PRACTICAL APPROACH (John M. Davis, Oxford Press, 2^{nd} ed. 2002); and CURRENT PROTOCOLS IN MOLECULAR BIOLOGY, supra, (2004). These protocols are routine procedures within the scope of one skilled in the art and from the teaching herein.

The methods disclosed in the present specification include, in part, introducing an expression construct into a heterologous cell. It is envisioned that any and all methods for introducing an expression construct disclosed in the present specification into a cell can be used. A cell disclosed in the present specification can maintain an expression construct transiently or stably. Stably-maintained constructs may be extra-chromosomal and replicate autonomously, or they may be integrated into the chromosomal material of the cell and replicate non-autonomously. Methods useful for introducing a nucleic acid molecule into a cell including, without limitation, calcium phosphate-mediated, DEAE dextran-mediated, lipid-mediated, polybrene-mediated, polylysine-mediated, viral-mediated, microinjection, protoplast fusion, biolistic, and electroporation, see, e.g., Introducing Cloned Genes into Cultured Mammalian Cells, pp. 16.1-16.62 (Sambrook & Russell, eds., Molecular Cloning A Laboratory Manual, Vol. 3, 3^{rd} ed. 2001). One skilled in the art understands that selection of a specific method to introduce an expression construct into a cell will depend, in part, on whether the cell will transiently contain an expression construct or whether the cell will stably contain an expression construct. These protocols are routine procedures within the scope of one skilled in the art and from the teaching herein.

It is envisioned that both cell-free and cell-based procedures can be used to produce an active BoNT/E using methods disclosed in the present specification. These procedures involve the use of well-characterized vectors, reagents, conditions and cells that are readily available from commercial vendors including, without limitation, BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; Roche Applied Science, Indianapolis, Ind.; and Stratagene, La Jolla, Calif. The selection and use of appropriate procedures to produce an active BoNT/E are described in e.g., PROTEIN EXPRESSION. A PRACTICAL APPROACH, supra, (1999) and Fernandez & Hoeffler, supra, (1999). These protocols are routine procedures within the scope of one skilled in the art and from the teaching herein.

One procedure of producing active BoNT/E employs a cell-free expression system such as, without limitation, prokaryotic extracts and eukaryotic extracts. Non-limiting examples of prokaryotic cell extracts include the RTS 100 E. coli HY Kit (Roche Applied Science, Indianapolis, Ind.), the ACTIVEPRO™ In Vitro Translation Kit (Ambion, Inc., Austin, Tex.), a prokaryotic S30-based in vitro translation and translation, the ECOPRO™ System, a prokaryotic S30-based in vitro translation and translation, (EMD Biosciences-Novagen, Madison, Wis.) and the EXPRESSWAY™ Plus Expression System, a prokaryotic S30-based in vitro translation and translation, (Invitrogen, Inc., Carlsbad, Calif.). Eukaryotic cell extract include, without limitation, the RTS 100 Wheat Germ CECF Kit (Roche Applied Science, Indianapolis, Ind.), the TnT® Coupled Wheat Germ Extract Systems, an eukaryotic wheat germ extract-based in vitro translation and translation, (Promega Corp., Madison, Wis.), the WHEAT GERM IVT™ Kit, an eukaryotic wheat germ extract-based in vitro translation and translation, (Ambion, Inc., Austin, Tex.), the RETIC LYSATE IVT™ Kit, an eukaryotic rabbit reticulocyte extract-based in vitro translation and translation, (Ambion, Inc., Austin, Tex.), the PROTEINSCRIPT® II System, an eukaryotic ITV-based in vitro translation and translation, (Ambion, Inc., Austin, Tex.) and the TNT® Coupled Reticulocyte Lysate Systems, an eukaryotic rabbit reticulocyte extract-based in vitro translation and translation, (Promega Corp., Madison, Wis.).

It is also envisioned that any of a variety of cell-based expression procedures are useful for expressing nucleic acid molecules encoding an active BoNT/E disclosed in the present specification. Examples included, without limitation, viral expression systems, prokaryotic expression systems, baculoviral expression systems, insect expression systems and mammalian expression systems. Viral expression systems include, without limitation, the-VIRAPOWER™ Lentiviral, a replication-incomplete, HIV-1-based lentivirus expression vector, (Invitrogen, Inc., Carlsbad, Calif.), the Adenoviral Expression Systems, a E1 and E3-deleted, pDEST-based expression vector controlled by a human cytomegalovirus (CMV) promoter, (Invitrogen, Inc., Carlsbad, Calif.), the ADEASY™ XL Adenoviral Vector System, an Ad5 virus-based expression vector, (Stratagene, La Jolla, Calif.) and the VIRAPORT® Retroviral Gene Expression System, a mammalian retrovirus expression vector, (Stratagene, La Jolla, Calif.). Non-limiting examples of prokaryotic expression systems include the CHAMPION™ pET Expression System, a prokaryotic expression vector, (EMD Biosciences-Novagen, Madison, Wis.), the TRIEX™ Bacterial Expression Systems, a prokaryotic expression vector, (EMD Biosciences-Novagen, Madison, Wis.), the QIAEXPRESS® Expression System, a prokaryotic expression vector, (QIAGEN, Inc.), and the AFFINITY® Protein Expression and Purification System, a prokaryotic expression vector, (Stratagene, La Jolla, Calif.). Yeast expression systems include, without limitation, the EASYSELECT™ Pichia Expression Kit, a yeast expression vector, (Invitrogen, Inc., Carlsbad, Calif.), the YES-ECHO™ Expression Vector Kits, a yeast expression vector, (Invitrogen, Inc., Carlsbad, Calif.) and the SPECTRA™ S. pombe Expression System, a yeast expression vector, (Invitrogen, Inc., Carlsbad, Calif.). Non-limiting examples of baculoviral expression systems include the BACULODIRECT™, a baculovirus-based expression vector, (Invitrogen, Inc., Carlsbad, Calif.), the BAC-TO-BAC®, a baculovirus-based expression vector, (Invitrogen, Inc., Carlsbad, Calif.), and the BD BACULOGOLD™, a baculovirus-based expression vector, (BD Biosciences-Pharmigen, San Diego, Calif.). Insect expression systems include, without limitation, the Drosophila Expression System (DES®) (Invitrogen, Inc., Carlsbad, Calif.), INSECTSELECT™ System, an insect expression vector, (Invitrogen, Inc., Carlsbad, Calif.) and INSECTDIRECT™ System, an insect expression vector, (EMD Biosciences-Novagen, Madison, Wis.). Non-limiting examples of mammalian expression systems include the T-REX™ (Tetracycline-Regulated Expression) System, a tetracycline-inducible mammalian expression vector, (Invitrogen, Inc., Carlsbad, Calif.), the FLP-IN™ T-REX™ System, a tetracycline-inducible mammalian expression vector, (Invitrogen, Inc., Carlsbad, Calif.), the pcDNA™ system, a constitutive mammalian expression vector, (Invitrogen, Inc., Carlsbad, Calif.), the-EXCHANGER® System, INTERPLAY™ mammalian TAP System, a mammalian expression vector, (Stratagene, La Jolla, Calif.), COMPLETE CONTROL® Inducible Mammalian Expression System, an ecdysone-inducible mammalian expression vector, (Stratagene, La Jolla, Calif.) and LACSWITCH® II Inducible Mammalian Expression System, a IPTG-inducible mammalian expression vector, (Stratagene, La Jolla, Calif.).

### EXAMPLES

The following non-limiting examples are provided for illustrative purposes only in order to facilitate a more complete understanding of disclosed embodiments and are in no way intended to limit any of the embodiments disclosed in the present specification.

### Example 1

### Selection of Nucleotide Alterations for an Open Reading Frame Providing Increased Expression of the Encoded Active BoNT/E in a Heterologous Cell

1. Manual Selection of Nucleotide Alterations

To determine codon use of a particular heterologous cell and how it compares to the codon usage found in C. botulinum, codon usage for C. botulinum and selected heterologous cells were tabulated using information obtained from the publicly maintained Codon Usage Database to facilitate comparisons among organisms (Table 1).

TABLE 1
Codon Usage Frequency

| | | Codon Usage Frequency (%) | | | | | | |
|---|---|---|---|---|---|---|---|---|
| Amino | | Clostridia | Escherichia | Pichia | Yarrowia | Spodoptera | Drosophila | Mus |
| Acid | Codon | botulinum | coli K12 | pastoris | lipolytica | frugiperda | melanogaster | musculus |
| Gly | GGG | 0.10 | 0.15 | 0.10 | 0.05 | 0.05 | 0.07 | 0.23 |
| Gly | GGA | 0.50 | 0.11 | 0.32 | 0.29 | 0.28 | 0.28 | 0.26 |
| Gly | GGT | 0.33 | 0.34 | 0.44 | 0.32 | 0.37 | 0.21 | 0.18 |
| Gly | GGC | 0.07 | 0.40 | 0.14 | 0.34 | 0.31 | 0.43 | 0.33 |
| Glu | GAG | 0.17 | 0.31 | 0.43 | 0.77 | 0.59 | 0.67 | 0.60 |
| Glu | GAA | 0.83 | 0.69 | 0.57 | 0.23 | 0.41 | 0.33 | 0.40 |
| Asp | GAT | 0.90 | 0.63 | 0.58 | 0.34 | 0.37 | 0.53 | 0.44 |
| Asp | GAC | 0.10 | 0.37 | 0.42 | 0.66 | 0.63 | 0.47 | 0.56 |
| Val | GTG | 0.07 | 0.37 | 0.19 | 0.33 | 0.35 | 0.47 | 0.46 |
| Val | GTA | 0.47 | 0.15 | 0.15 | 0.05 | 0.15 | 0.11 | 0.12 |
| Val | GTT | 0.45 | 0.26 | 0.42 | 0.25 | 0.20 | 0.18 | 0.17 |
| Val | GTC | 0.02 | 0.22 | 0.23 | 0.37 | 0.30 | 0.24 | 0.25 |
| Ala | GCG | 0.04 | 0.35 | 0.06 | 0.08 | 0.17 | 0.19 | 0.10 |
| Ala | GCA | 0.46 | 0.21 | 0.24 | 0.11 | 0.15 | 0.17 | 0.23 |
| Ala | GCT | 0.45 | 0.16 | 0.45 | 0.35 | 0.36 | 0.19 | 0.29 |
| Ala | GCC | 0.06 | 0.27 | 0.26 | 0.46 | 0.31 | 0.45 | 0.38 |
| Arg | AGG | 0.12 | 0.02 | 0.15 | 0.04 | 0.21 | 0.11 | 0.22 |
| Arg | AGA | 0.73 | 0.04 | 0.47 | 0.13 | 0.16 | 0.09 | 0.21 |
| Ser | AGT | 0.30 | 0.15 | 0.15 | 0.07 | 0.11 | 0.14 | 0.15 |
| Ser | AGC | 0.06 | 0.28 | 0.09 | 0.11 | 0.17 | 0.25 | 0.24 |
| Lys | AAG | 0.19 | 0.23 | 0.54 | 0.85 | 0.69 | 0.71 | 0.61 |
| Lys | AAA | 0.81 | 0.77 | 0.46 | 0.15 | 0.31 | 0.29 | 0.39 |
| Asn | AAT | 0.90 | 0.45 | 0.47 | 0.17 | 0.29 | 0.44 | 0.43 |
| Asn | AAC | 0.10 | 0.55 | 0.53 | 0.83 | 0.71 | 0.56 | 0.57 |
| Met | ATG | 1.00 | 1.00 | 1.00 | 1.00 | 1.00 | 1.00 | 1.00 |
| Ile | ATA | 0.52 | 0.07 | 0.18 | 0.03 | 0.12 | 0.19 | 0.16 |
| Ile | ATT | 0.43 | 0.51 | 0.50 | 0.44 | 0.29 | 0.34 | 0.34 |
| Ile | ATC | 0.05 | 0.42 | 0.32 | 0.53 | 0.60 | 0.47 | 0.51 |
| Thr | ACG | 0.04 | 0.27 | 0.11 | 0.11 | 0.16 | 0.26 | 0.11 |
| Thr | ACA | 0.44 | 0.13 | 0.24 | 0.13 | 0.21 | 0.19 | 0.29 |
| Thr | ACT | 0.46 | 0.17 | 0.40 | 0.26 | 0.27 | 0.17 | 0.25 |
| Thr | ACC | 0.06 | 0.43 | 0.25 | 0.50 | 0.36 | 0.38 | 0.35 |
| Trp | TGG | 1.00 | 1.00 | 1.00 | 1.00 | 1.00 | 1.00 | 1.00 |
| End | TGA | 0.03 | 0.29 | 0.18 | 0.15 | 0.16 | 0.26 | 0.50 |
| Cys | TGT | 0.80 | 0.45 | 0.66 | 0.45 | 0.35 | 0.29 | 0.48 |
| Cys | TGC | 0.20 | 0.55 | 0.34 | 0.55 | 0.65 | 0.71 | 0.52 |
| End | TAG | 0.23 | 0.07 | 0.28 | 0.39 | 0.16 | 0.33 | 0.23 |
| End | TAA | 0.74 | 0.64 | 0.54 | 0.46 | 0.69 | 0.41 | 0.27 |
| Tyr | TAT | 0.90 | 0.57 | 0.44 | 0.17 | 0.25 | 0.37 | 0.42 |
| Tyr | TAC | 0.10 | 0.43 | 0.56 | 0.83 | 0.75 | 0.63 | 0.58 |
| Leu | TTG | 0.10 | 0.13 | 0.33 | 0.09 | 0.20 | 0.18 | 0.13 |
| Leu | TTA | 0.65 | 0.13 | 0.15 | 0.01 | 0.07 | 0.05 | 0.06 |
| Phe | TTT | 0.88 | 0.57 | 0.54 | 0.37 | 0.24 | 0.37 | 0.43 |
| Phe | TTC | 0.12 | 0.43 | 0.46 | 0.63 | 0.76 | 0.63 | 0.57 |
| Ser | TCG | 0.02 | 0.15 | 0.09 | 0.16 | 0.13 | 0.20 | 0.05 |
| Ser | TCA | 0.28 | 0.12 | 0.19 | 0.08 | 0.15 | 0.09 | 0.14 |
| Ser | TCT | 0.30 | 0.15 | 0.29 | 0.28 | 0.19 | 0.08 | 0.19 |
| Ser | TCC | 0.04 | 0.15 | 0.20 | 0.31 | 0.25 | 0.24 | 0.22 |
| Arg | CGG | 0.01 | 0.10 | 0.05 | 0.11 | 0.05 | 0.15 | 0.19 |
| Arg | CGA | 0.04 | 0.06 | 0.11 | 0.55 | 0.07 | 0.15 | 0.12 |
| Arg | CGT | 0.09 | 0.38 | 0.16 | 0.10 | 0.26 | 0.16 | 0.09 |
| Arg | CGC | 0.01 | 0.40 | 0.05 | 0.07 | 0.24 | 0.33 | 0.18 |
| Gln | CAG | 0.14 | 0.65 | 0.39 | 0.82 | 0.60 | 0.70 | 0.75 |
| Gln | CAA | 0.86 | 0.35 | 0.61 | 0.18 | 0.40 | 0.30 | 0.25 |
| His | CAT | 0.87 | 0.57 | 0.54 | 0.32 | 0.32 | 0.40 | 0.40 |
| His | CAC | 0.13 | 0.43 | 0.46 | 0.68 | 0.68 | 0.60 | 0.60 |
| Leu | CTG | 0.01 | 0.50 | 0.16 | 0.38 | 0.31 | 0.43 | 0.40 |
| Leu | CTA | 0.10 | 0.04 | 0.11 | 0.05 | 0.07 | 0.09 | 0.08 |
| Leu | CTT | 0.13 | 0.10 | 0.16 | 0.18 | 0.13 | 0.10 | 0.13 |
| Leu | CTC | 0.01 | 0.10 | 0.08 | 0.29 | 0.22 | 0.15 | 0.20 |
| Pro | CCG | 0.03 | 0.52 | 0.09 | 0.09 | 0.16 | 0.29 | 0.10 |
| Pro | CCA | 0.44 | 0.19 | 0.40 | 0.10 | 0.23 | 0.25 | 0.28 |
| Pro | CCT | 0.46 | 0.16 | 0.35 | 0.32 | 0.30 | 0.13 | 0.30 |
| Pro | CCC | 0.07 | 0.13 | 0.15 | 0.49 | 0.31 | 0.33 | 0.31 |
| | | Codon Usage Frequency (%) | | | | | | |
| Amino | | Rattus | Cricetulus | Sus | Bos | Equus | Cercopithecus | Homo |
| Acid | Codon | norvegicus | griseus | scrofa | taurus | caballus | aethiops | sapiens |
| Gly | GGG | 0.24 | 0.21 | 0.26 | 0.25 | 0.24 | 0.26 | 0.25 |
| Gly | GGA | 0.25 | 0.25 | 0.23 | 0.24 | 0.23 | 0.24 | 0.25 |
| Gly | GGT | 0.17 | 0.20 | 0.14 | 0.16 | 0.17 | 0.15 | 0.16 |
| Gly | GGC | 0.34 | 0.34 | 0.37 | 0.35 | 0.37 | 0.35 | 0.34 |
| Glu | GAG | 0.61 | 0.60 | 0.64 | 0.60 | 0.64 | 0.62 | 0.58 |
| Glu | GAA | 0.39 | 0.40 | 0.36 | 0.40 | 0.36 | 0.38 | 0.42 |
| Asp | GAT | 0.42 | 0.47 | 0.39 | 0.42 | 0.41 | 0.42 | 0.46 |
| Asp | GAC | 0.58 | 0.53 | 0.61 | 0.58 | 0.59 | 0.58 | 0.54 |
| Val | GTG | 0.48 | 0.46 | 0.51 | 0.49 | 0.50 | 0.46 | 0.47 |
| Val | GTA | 0.11 | 0.12 | 0.08 | 0.10 | 0.08 | 0.08 | 0.12 |
| Val | GTT | 0.16 | 0.18 | 0.14 | 0.16 | 0.14 | 0.17 | 0.18 |
| Val | GTC | 0.26 | 0.24 | 0.27 | 0.26 | 0.28 | 0.29 | 0.24 |
| Ala | GCG | 0.10 | 0.07 | 0.12 | 0.11 | 0.12 | 0.10 | 0.11 |
| Ala | GCA | 0.22 | 0.24 | 0.18 | 0.20 | 0.18 | 0.19 | 0.23 |
| Ala | GCT | 0.28 | 0.33 | 0.24 | 0.26 | 0.25 | 0.26 | 0.26 |
| Ala | GCC | 0.40 | 0.37 | 0.46 | 0.43 | 0.44 | 0.45 | 0.40 |
| Arg | AGG | 0.21 | 0.19 | 0.20 | 0.21 | 0.22 | 0.20 | 0.21 |
| Arg | AGA | 0.19 | 0.19 | 0.19 | 0.20 | 0.20 | 0.16 | 0.21 |
| Ser | AGT | 0.15 | 0.16 | 0.12 | 0.14 | 0.13 | 0.14 | 0.15 |
| Ser | AGC | 0.25 | 0.22 | 0.27 | 0.25 | 0.26 | 0.26 | 0.24 |
| Lys | AAG | 0.63 | 0.61 | 0.63 | 0.61 | 0.63 | 0.61 | 0.57 |
| Lys | AAA | 0.37 | 0.39 | 0.37 | 0.39 | 0.37 | 0.39 | 0.43 |
| Asn | AAT | 0.40 | 0.45 | 0.39 | 0.40 | 0.39 | 0.41 | 0.47 |
| Asn | AAC | 0.60 | 0.55 | 0.61 | 0.60 | 0.61 | 0.59 | 0.53 |
| Met | ATG | 1.00 | 1.00 | 1.00 | 1.00 | 1.00 | 1.00 | 1.00 |
| Ile | ATA | 0.14 | 0.14 | 0.13 | 0.14 | 0.13 | 0.12 | 0.16 |
| Ile | ATT | 0.32 | 0.35 | 0.29 | 0.33 | 0.29 | 0.30 | 0.36 |
| Ile | ATC | 0.54 | 0.51 | 0.57 | 0.53 | 0.53 | 0.58 | 0.48 |
| Thr | ACG | 0.12 | 0.08 | 0.15 | 0.13 | 0.13 | 0.18 | 0.12 |
| Thr | ACA | 0.28 | 0.29 | 0.22 | 0.25 | 0.22 | 0.27 | 0.28 |
| Thr | ACT | 0.23 | 0.26 | 0.20 | 0.22 | 0.22 | 0.22 | 0.24 |
| Thr | ACC | 0.37 | 0.37 | 0.43 | 0.39 | 0.43 | 0.34 | 0.36 |
| Trp | TGG | 1.00 | 1.00 | 1.00 | 1.00 | 1.00 | 1.00 | 1.00 |
| End | TGA | 0.49 | 0.49 | 0.55 | 0.49 | 0.42 | 0.48 | 0.49 |
| Cys | TGT | 0.44 | 0.47 | 0.39 | 0.42 | 0.43 | 0.39 | 0.45 |
| Cys | TGC | 0.56 | 0.53 | 0.61 | 0.58 | 0.57 | 0.61 | 0.55 |
| End | TAG | 0.23 | 0.26 | 0.19 | 0.23 | 0.23 | 0.32 | 0.23 |
| End | TAA | 0.28 | 0.25 | 0.25 | 0.29 | 0.35 | 0.20 | 0.28 |
| Tyr | TAT | 0.40 | 0.44 | 0.36 | 0.39 | 0.35 | 0.41 | 0.44 |
| Tyr | TAC | 0.60 | 0.56 | 0.64 | 0.61 | 0.65 | 0.59 | 0.56 |
| Leu | TTG | 0.12 | 0.15 | 0.11 | 0.12 | 0.11 | 0.12 | 0.13 |
| Leu | TTA | 0.06 | 0.06 | 0.05 | 0.06 | 0.05 | 0.06 | 0.07 |
| Phe | TTT | 0.41 | 0.47 | 0.38 | 0.41 | 0.39 | 0.40 | 0.46 |
| Phe | TTC | 0.59 | 0.53 | 0.62 | 0.59 | 0.61 | 0.60 | 0.54 |
| Ser | TCG | 0.06 | 0.05 | 0.06 | 0.06 | 0.06 | 0.05 | 0.06 |
| Ser | TCA | 0.14 | 0.14 | 0.12 | 0.13 | 0.12 | 0.13 | 0.15 |
| Ser | TCT | 0.19 | 0.22 | 0.17 | 0.18 | 0.18 | 0.19 | 0.19 |
| Ser | TCC | 0.23 | 0.22 | 0.26 | 0.23 | 0.25 | 0.23 | 0.22 |
| Arg | CGG | 0.20 | 0.20 | 0.21 | 0.20 | 0.18 | 0.22 | 0.21 |
| Arg | CGA | 0.12 | 0.14 | 0.10 | 0.11 | 0.10 | 0.11 | 0.11 |
| Arg | CGT | 0.09 | 0.11 | 0.07 | 0.08 | 0.10 | 0.08 | 0.08 |
| Arg | CGC | 0.19 | 0.17 | 0.22 | 0.20 | 0.20 | 0.22 | 0.19 |
| Gln | CAG | 0.76 | 0.77 | 0.78 | 0.76 | 0.76 | 0.73 | 0.74 |
| Gln | CAA | 0.24 | 0.23 | 0.22 | 0.24 | 0.24 | 0.27 | 0.26 |
| His | CAT | 0.38 | 0.44 | 0.35 | 0.36 | 0.38 | 0.36 | 0.42 |
| His | CAC | 0.62 | 0.56 | 0.65 | 0.64 | 0.62 | 0.64 | 0.58 |
| Leu | CTG | 0.42 | 0.40 | 0.45 | 0.43 | 0.46 | 0.41 | 0.40 |
| Leu | CTA | 0.07 | 0.08 | 0.05 | 0.06 | 0.06 | 0.06 | 0.07 |
| Leu | CTT | 0.12 | 0.13 | 0.11 | 0.12 | 0.11 | 0.15 | 0.13 |
| Leu | CTC | 0.21 | 0.19 | 0.23 | 0.21 | 0.22 | 0.20 | 0.20 |
| Pro | CCG | 0.11 | 0.08 | 0.14 | 0.13 | 0.11 | 0.11 | 0.11 |
| Pro | CCA | 0.27 | 0.28 | 0.24 | 0.25 | 0.23 | 0.33 | 0.27 |
| Pro | CCT | 0.30 | 0.32 | 0.26 | 0.27 | 0.30 | 0.24 | 0.28 |
| Pro | CCC | 0.32 | 0.32 | 0.37 | 0.35 | 0.35 | 0.33 | 0.33 |

To determine G+C content of a particular heterologous cell and how it compares to the codon usage found in C. botulinum, G+C content for C. botulinum and selected heterologous cells were tabulated using information obtained from the publicly maintained Codon Usage Database to facilitate comparisons among organisms (Table 2).

TABLE 2
G + C content

| | | First Codon | Second Codon | Third Codon |
|---|---|---|---|---|
| | Total | Position G + C | Position | Position |
| | G + C Content | Content | G + C Content | G + C Content |
| Organism | (%) | (%) | (%) | (%) |
| Clostridium botulinum | 25.29 | 33.44 | 28.38 | 14.04 |
| Escherichia coli | 51.80 | 58.89 | 40.72 | 55.79 |
| Pichia pastoris | 42.99 | 49.16 | 37.49 | 42.32 |
| Yarrowia lipolytica | 54.69 | 58.17 | 41.18 | 64.71 |
| Zea mays | 54.60 | 57.46 | 43.03 | 63.31 |
| Spodoptera frugiperda | 51.44 | 53.92 | 39.52 | 60.88 |
| Drosophila melanogaster | 53.99 | 55.90 | 41.51 | 64.57 |
| Mus musculus | 52.33 | 55.57 | 42.19 | 59.24 |
| Rattus norvegicus | 52.82 | 55.64 | 41.64 | 61.19 |
| Cricetulus griseus | 51.26 | 55.29 | 40.43 | 58.07 |
| Sus scrofa | 54.68 | 56.47 | 41.95 | 65.63 |
| Bos taurus | 53.14 | 55.43 | 41.46 | 62.53 |
| Equus caballus | 53.63 | 55.96 | 40.71 | 64.21 |
| Cercopithecus aethiops | 52.81 | 53.80 | 42.36 | 62.26 |
| Homo sapiens | 52.54 | 56.10 | 42.55 | 58.99 |

Using Tables 1 and 2, one skilled in the art can manually select which nucleotides to alter to the open reading frame of SEQ ID NO: 3 so that the open reading frame now provides synonymous codons preferred by the heterologous cell selected to express this open reading frame and increase the G+C content to better match the G+C content of this heterologous cell.

2. Computer-Assisted Selection of Nucleotide Alterations

To alter the open reading frame of SEQ ID NO: 3 in order to provide increased expression of the encoded BoNT/E in a heterologous cell, synonymous codon usage for each organism was determined using the publicly available Backtranslate Tool, version 2 (Entelechon, GmbH, Regensburg, Germany, at URL address entelechon.com/eng/backtranslation). The active BoNT/E amino acid sequence of SEQ ID NO: 1 was submitted to this web-based program and prospective modified open reading frames were generated. These modified sequences were subsequently analyzed for G+C content, and substitutions that better matched the G+C content of a specific heterologous cell were made. This procedure resulted in the modified open reading frames SEQ ID NO: 5 through SEQ ID NO: 97, each encoding an active BoNT/E of SEQ ID NO: 1, but optimized to be expressed in a heterologous cell.

### Example 2

### Synthesis of a Nucleic Acid Molecule

A nucleic acid molecule of SEQ ID NO: 98 encoding a BoNT/E was modified so that particular synonymous codons preferred by E. coli were incorporated and the G+C content was increased from about 25% to approximately 40%. Initially, an algorithm generated the modified open reading frame of SEQ ID NO: 98 that encoded the BoNT/E of SEQ ID NO: 2 (BLUEHERON® Biotechnology, Bothell Wash.). This program 1) reduced the mRNA secondary structure (based on a free energy calculation) of the nucleic acid molecule and 2) altered the synonymous codon usage of the open reading frame of the nucleic acid molecule to an overall codon usage preferred by E. coli. The algorithm uses a statistical model to search for improved solutions (i.e., combinations of representative codon usage and lower free energy) through an iterative process. This sequence was then modified by one skilled in the art at Allergan, Inc. to add unique restriction endonuclease sites at the 5′-termini (e.g., EcoRV, BamHI, EcoRI, SacI and NdeI) and 3′-termini (e.g., SalI, HindIII, NotI, EagI, XhoI and AvaI) of the nucleic acid molecule in order to facilitate cloning into expression vectors, reduce polymononucleotide regions and remove internal regulatory or structural site sequences.

Based on this sequence information above, BLUEHERON® Biotechnology synthesized a nucleic acid molecule of SEQ ID NO: 98, designated BoNT/E (8m). Oligonucleotides of 20 to 50 bases in length were synthesized using standard phosphoramidite synthesis. These oligonucleotides were hybridized into double stranded duplexes that were ligated together to assemble the full-length nucleic acid molecule. This nucleic acid molecule was cloned using standard molecular biology methods into a pUCBHB1 vector at the SmaI site to generate pUCBHB1-BoNT/E (8m). The synthesized nucleic acid molecule was verified by sequencing using BIG DYE TERMINATOR™ Chemistry 3.1, a fluorescently-labeled dideoxynucleotide chain-terminator sequencing method, (Applied Biosystems, Foster City, Calif.) and an ABI 3100 sequencer (Applied Biosystems, Foster City, Calif.).

### Example 3

### Construction of pET28a/His-BoNT/E (8m)

To construct pET28a/His-BoNT/E (8m), a pUCBHB1/BoNT/E (8m) construct was digested with NdeI and HindIII at 37° C. for 2.5 hours to excise the BoNT/E (8m) insert. The resulting restriction fragment was purified by the QIAquick Gel Extraction Kit (QIAGEN, Inc., Valencia, Calif.), and the fragment containing the entire open reading frame was subcloned into the pET28a vector (EMD Biosciences-Novagen, Madison, Wis.) that had been digested with restriction endonucleases NdeI and HindIII. The fragment and vector were ligated using T4 DNA ligase protocol to yield pET28a/His-BoNT/E (8m). Both 1 μL and 2 μL samples from this ligation mixture were transformed by a standard heat-shock protocol into two separate vials of competent TOP10 cells (Invitrogen, Inc, Carlsbad, Calif.), plated onto 1.5% Luria-Bertani agar plates (pH 7.0) containing 50 μg/mL of Kanamycin, and placed in a 37° C. incubator for overnight growth. Candidate expression constructs were selected as Kanamycin-resistant colonies. Resistant colonies were used to inoculate 2 mL of Luria-Bertani media containing 50 μg/mL of Kanamycin that were then grown in a 37° C. incubator, shaking at 250 rpm, for 5 hours. The bacteria cells were harvested by microcentrifugation and the plasmid DNA was isolated using QIAGEN miniprep kits (QIAGEN, Inc., Valencia, Calif.). Candidate expression constructs were screened by restriction digestion with NdeI and HindIII to determine the presence and orientation of the correct insert fragment. Cultures containing the desired expression construct were used to inoculate 1 L baffled flasks containing 200 mL of Luria-Bertani media containing 50 μg/mL of Kanamycin and placed in a 37° C. incubator, shaking at 250 rpm, for overnight growth. Purified plasmid DNA corresponding to an expression construct was isolated using the QIAGEN Maxi-prep method (QIAGEN, Inc., Valencia, Calif.) and sequenced to verify that the correct expression construct was made (service contract with Sequetech Corp., Mountain View, Calif.). This cloning strategy yielded a prokaryotic expression construct encoding a BoNT/E (8m) peptide containing an amino-terminal thrombin cleavable polyhistidine affinity binding peptide.

To test for enzymatic activity of His-BoNT/E (8m), the light chain from His-BoNT/E (8m) was tested using a SNAP-25 Cleavage Assay. Because of regulatory and safety considerations, initial activity assays were performed using pQBI25/GFP-LC-BoNT/E (8m), a construct comprising amino acids 3 to 422 of SEQ ID NO: 2, the light chain of BoNT/E (8m), operationally linked to an amino-terminal Green Fluorescent Protein (GFP). To test for enzymatic activity using a SNAP-25 Cleavage Assay, about 1.0×10⁶ SH-SY5Y cells were plated in a 60 mm tissue culture dish containing 5 mL of complete Dulbecco's Modified Eagle Media (DMEM), supplemented with 10% fetal bovine serum (FBS), 1× penicillin/streptomycin solution (Invitrogen, Inc, Carlsbad, Calif.) and 1× MEM non-essential amino acids solution (Invitrogen, Inc, Carlsbad, Calif.), and grown in a 37° C. incubator under 5% carbon dioxide until cells reach a density of about 5×10⁶ cells/ml (6-16 hours). A 500 μL transfection solution is prepared by adding 250 μL of OPTI-MEM Reduced Serum Medium containing 15 μL of LIPOFECTAMINE 2000, a cationic liposome based reagent, (Invitrogen, Carlsbad, Calif.) incubated at room temperature for 5 minutes to 250 μL of OPTI-MEM Reduced Serum Medium containing 5 μg of pQBI25/GFP-LC-BoNT/E. This transfection was incubated at room temperature for approximately 20 minutes. The complete, supplemented DMEM media was replaced with 2 mL of OPTI-MEM Reduced Serum Medium and the 500 μL transfection solution was added to the SH-SY5Y cells and the cells incubated in a 37° C. incubator under 5% carbon dioxide for approximately 6 to 18 hours. Transfection media was replaced with 3 mL of fresh complete, supplemented DMEM and incubate cells in a 37° C. incubator under 5% carbon dioxide for an additional 24 hours. Cells were harvest by rinsing cells once with 3.0 mL of 100 mM phosphate-buffered saline, pH 7.4 and rinsed cells were lysed with 500 μL of Lysis Buffer containing 50 mM N-(2-hydroxyethyl)piperazine-N′-(2-ethanesulfonic acid) (HEPES), 150 mM sodium chloride, 1.5 mM magnesium chloride, 1 mM ethylene glycol bis(β-aminoethyl ether) N,N, N′,N′-tetraacetic acid (EGTA), 10% (v/v) glycerol and 1% (v/v) TRITON-X® 100 (4-octylphenol polyethoxylate) at 4° C. with rotation for 60 minutes. Lysed cells were centrifuged (5000 rpm at 4° C. for 10 min) to pellet debris and the supernatant was transferred to fresh siliconized tubes.

The protein concentration of a sample was measured by a Bradford dye assay and 1×LDS Sample Buffer was added to bring the protein concentration to 1 mg/ml. Protein samples were then added to 2×LDS Sample Buffer (Invitrogen, Inc, Carlsbad, Calif.) and separated by MOPS polyacrylamide gel electrophoresis using NUPAGE® Novex 4-12% Bis-Tris precast polyacrylamide gels (Invitrogen, Inc, Carlsbad, Calif.) under denaturing, reducing conditions. Separated peptides were transferred from the gel onto polyvinylidene fluoride (PVDF) membranes (Invitrogen, Inc, Carlsbad, Calif.) by Western blotting using a TRANS-BLOT® SD semi-dry electrophoretic transfer cell apparatus (Bio-Rad Laboratories, Hercules, Calif.). PVDF membranes were blocked by incubating at room temperature for 2 hours in a solution containing 25 mM Tris-Buffered Saline (25 mM 2-amino-2-hydroxymethyl-1,3-propanediol hydrochloric acid (Tris-HCl), pH 7.4, 137 mM sodium chloride, 2.7 mM potassium chloride), 0.1% TWEEN-20®, polyoxyethylene (20) sorbitan monolaureate, 2% (w/v) bovine serum albumin, 5% (w/v) nonfat dry milk. Blocked membranes were incubated at 4° C. for overnight in Tris-Buffered Saline TWEEN-20® (25 mM Tris-Buffered Saline, 0.1% TWEEN-20®, polyoxyethylene (20) sorbitan monolaureate) containing the following primary antibodies as a probe: a 1:50,000 dilution of mouse monoclonal anti-BoNT/E SMI-81 antibody (Sternberger Monoclonals, Lutherville, Md.). Primary antibody probed blots were washed three times for 15 minutes each time in Tris-Buffered Saline TWEEN-20®. Washed membranes were incubated at room temperature for 2 hours in Tris-Buffered Saline TWEEN-20® containing a 1:20,000 dilution of goat polyclonal anti-mouse immunoglobulin G, heavy and light chains (IgG, H+L) antibody conjugated to horseradish peroxidase (HRP; Pierce Biotechnology, Inc., Rockford, Ill.) as a secondary antibody. Secondary antibody-probed blots were washed three times for 15 minutes each time in Tris-Buffered Saline TWEEN-20®. Signal detection of labeled His-iBoNT/E was visualized using the ECL Plus™ Western Blot Detection System (Amersham Biosciences, Piscataway, N.J.) and imaged with a Typhoon 9410 Variable Mode Imager (Amersham Biosciences, Piscataway, N.J.) for quantification of peptide expression levels.

Unexpectedly, this initial analysis revealed that while the positive controls showed significant protease activity, GFP-LC-BoNT/E expressed at the same level as the controls from modified nucleic acid molecules derived from SEQ ID NO: 98 did not exhibit appreciable levels of enzymatic activity. Sequence alignment of the full-length BoNT/E revealed eight non-conserved amino acid polymorphisms that were unique to the native C. botulinum sequence deposited in GenBank as accession # X62089 (SEQ ID NO: 2) relative to another native C. botulinum sequence deposited as accession # X62683 (SEQ ID NO: 1). There were three amino acid substitutions located in the LC, while four substitutions and a single amino acid deletion were present in the HC. The amino acid changes that were unique to the Genbank C. botulinum (accession # X62089; SEQ ID NO: 2) are summarized in Table 3.

Using a SNAP-25 Cleavage Assay as described above, the presence of either G177 or A340 in the LC resulted in enzymatic inactivity of the His-BoNT/E (8m). Furthermore, any combination of the two LC amino acid mutations from SEQ ID NO: 2 inactivated the BoNT/E (i.e., G177/5198, G177/A340, 5198/A340). Thus, the nucleic acid molecule of SEQ ID NO: 98 encodes an inactive BoNT/E (SEQ ID NO: 2) containing eight mutations and is referred to as: BoNT/E (G177R/S198C/A340R/L7731/L963F/Q964E/A967R/ΔN1196) or BoNT/E (8m).

TABLE 3
Eight mutations found in BoNT/E

| | BoNT/E | BoNT/E | | |
|---|---|---|---|---|
| Position | SEQ ID NO: 1 | SEQ ID NO: 2 | Abbreviation | Conserved |
| 177 (LC) | Glycine | Arginine | G177R | No |
| 198 (LC) | Serine | Cysteine | S198C | Weak |
| 340 (LC) | Alanine | Arginine | A340R | No |
| 773 (HC) | Leucine | Isoleucine | L773I | Strong |
| 963 (HC) | Leucine | Phenylalanine | L963F | No |
| 964 (HC) | Glutamine | Glutamate | Q964E | Weak |
| 967 (HC) | Alanine | Arginine | A967R | No |
| 1196 (HC) | Asparagine | Deleted | ΔN1196 | No |

### Example 4

### Construction of pET28a/His-iBoNT/E (H216Y) and pET28a/His-iBoNT/E (E213Q)

Because of regulatory and safety considerations, initial expression of a construct comprising a modified open reading frame encoding BoNT/E was performed using enzymatically inactive BoNT/E (iBoNT/E). These initial expression attempts allowed development of the protocols and strategies necessary for expressing the constructs encoding an active BoNT/E. Several iBoNT/Es were designed based on the knowledge that mutation of the zinc binding motif within the LC disrupts enzymatic activity. Substitution of residue Histidine-227 in BoNT/A with tyrosine (H227Y) is inactivating, see, e.g., Zhou et al., supra, (1995). Since the zinc binding motif of the BoNT light chain endopeptidases is absolutely conserved, the equivalent BoNT/E point mutation was designed to yield iBoNT/E (H216Y) (numbering based on the native BoNT/E sequence without any affinity binding peptide). A second point mutation, one in which glutamine replaces Glutamate-213 (E213Q), was also constructed. Unlike the H216Y mutant, in which a zinc binding residue is mutated, the E213Q mutation replaces the residue responsible for coordinating and activating the nucleophilic water molecule that adds to the scissile peptide bond. Both of these inactivating mutations are within the highly conserved zinc binding motif (Table 4).

TABLE 4
Zinc-binding motif inactivating mutations

| Consensus motif: | HExxH |
|---|---|
| Native BoNT/E: | HELIH |
| iBoNT/E(H216Y) | HELIY |
| iBoNT/E(E213Q) | HQLIH |

The pET28a/His-BoNT/E (8m) of Example 3 was used as the starting construct for a series of site-directed in vitro mutagenesis experiments that resulted in the construction of the prokaryotic expression constructs pET28a/His-iBoNT/E (H216Y) and pET28a/His-iBoNT/E (E213Q). These experiments both corrected the eight mutations discussed in Example 3 and introduced one of the two zinc binding motif mutations. A 50 μL reaction was assembled with the pET28a/His-BoNT/E (8m) expression construct as a template, primers specified below and reagents included with the QuickChange® II XL Site-Directed Mutagenesis kit (Stratagene, La Jolla, Calif.). The polymerase chain reaction (PCR) mix contained 5 μL of 10× Buffer, 1 μL of deoxyribonucleotides (dNTPs), 1 μL of PfuUltra™ High Fidelity DNA polymerase (2.5 units/μL), 125 ng of each primer, 100 ng of template DNA, and nuclease-free water to a final volume of 50 μL. The thermocycler conditions were: one cycle of 95° C. for 60 seconds; 16 cycles of 95° C. for 30 seconds, 55° C. for 60 seconds, and 72° C. for 10 minutes; one cycle of 72° C. for 5 minutes; and 4° C. to hold. Following thermocycling, 1 μL of DpnI restriction enzyme (Stratagene, La Jolla, Calif.) was added to the reaction and incubated for 1 hour at 37° C. to digest the template DNA. The reaction was purified by QIAquick kit (QIAGEN, Inc., Valencia, Calif.) and analysis by agarose gel electrophoresis showed that the reaction produced full-length plasmid. The mutagenesis products were transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Candidate mutagenesis constructs were isolated as Ampicillin resistant colonies and analyzed using an alkaline lysis plasmid mini-preparation procedure to isolate the expression construct and restriction endonuclease digests to determine the presence of the insert. The incorporation of the point mutation was determined by sequence analysis (service contract with Sequetech Corp., Mountain View, Calif.) of candidate plasmid constructs.

In the initial round of site-directed mutagenesis the following oligonucleotide primer pairs were used to alter the nucleic acid molecule encoding iBoNT/E (H216Y): R177G Primer Pair, sense oligonucleotide, 5′-TATATGCCGTCGAACCATGGCTTTGGCTCAATCGCAATTG-3′ (SEQ ID NO: 99) and antisense oligonucleotide, 5′-CAATTGCGATTGAGCCAAAGCCATGGTTCGACGGCATATA-3′ (SEQ ID NO: 100); C198S Primer Pair, sense oligonucleotide, 5′-CGTTTTAACGACAACAGCATGAATGAATTTAT CC-3′ (SEQ ID NO: 101) and antisense oligonucleotide, 5′-GGATAAATTCATTCATGCTGTTGTCGTTAA ACG-3′ (SEQ ID NO: 102); R340A Primer Pair, sense oligonucleotide, 5′-CCTTCACCGAATTTGATTTGG CCACCAAATTCCAGGTCAA-3′ (SEQ ID NO: 103) and antisense oligonucleotide, 5′-TTGACCTGGAATT TGGTGGCCAAATCAAATTCGGTGAAGG-3′ (SEQ ID NO: 104); 1773L Primer Pair, sense oligonucleotide, 5′-CTATTTCCTATTTGATGAAACTTATCAATGAAGTCAAA-3′ (SEQ ID NO: 105) and antisense, 5′-TTTGACTTCATTGATAAGTTTCATCAAATAGGAAATAG-3′ (SEQ ID NO: 106); F963L/E964Q/R967A Primer Pair, sense primer, 5′-TATCTGGA CTCTTCAGGACAATGCTGGTATCAACCAAAA ATTAGC-3′ (SEQ ID NO: 107) and antisense oligonucleotide 5′-GCTAATTTTTGGTTGATACCAGCATTG TCCTGAAGAGTCCAGATA-3′ (SEQ ID NO: 108); +N1196 Primer Pair, sense oligonucleotide, 5′-GTTAT GAACTCGGTCGGCAACAATTGTACTATGAAT-3′ (SEQ ID NO: 109) and antisense oligonucleotide, 5′-ATTCATAGTACAATTGTTGCCGACCGAGTTCATAAC-3′ (SEQ ID NO: 110) The nucleotides that were changed to correct the coding sequence are shown in bold and underlined. Sequence analysis of the resulting plasmid revealed that three corrections (C198S, R340A and Δ1196N) were incorporated to yield pET28a/His-iBoNT/E (G177R/L773I/L963F/Q964E/A967R).

In the second round of site-directed mutagenesis the zinc-binding residue, His-216, was mutated to tyrosine using the following oligonucleotide primer pair to yield pET28a/His-iBoNT/E (G177R/H216Y/L7731/L963F/Q964E/A967R): H216Y Primer Pair, sense oligonucleotide, 5′-GCTGACTTTGATGCATGAA CTGATCTATAGCTTGCACGGCCTG-3′ (SEQ ID NO: 111) and antisense oligonucleotide, 5′-CAGGCCG TGCAAGCTATAGATCAGTTCATGCATCAAAGTCAGC-3′ (SEQ ID NO: 112). The nucleotides that were mutated are shown in bold and underlined.

In a third round of site-directed mutagenesis, the 1773L primer pair (SEQ ID NO: 105 and SEQ ID NO: 106) and the F963L/E964Q/R967A primer pair (SEQ ID NO: 107 and SEQ ID NO: 108) were utilized to yield pET28a/His-iBoNT/E (G177R/H216Y). In the final round of site-directed mutagenesis, the R177G primer pair (SEQ ID NO: 99 and SEQ ID NO: 100) was used to correct the G177R substitution and produce the expression construct pET28a/His-iBoNT/E (H216Y), containing the modified nucleic acid sequence of SEQ ID NO: 118 encoding iBoNT/E (H216Y).

The plasmid coding for a second iBoNT/E, iBoNT/E (E213Q), was prepared by site-directed mutagenesis of pET28a/His-iBoNT/E (H216Y) using the procedure as described above. Correction of the inactivating H216Y mutation and incorporation of the inactivating E213Q mutation were accomplished in a single site-directed mutagenesis step using the procedure described above and the following two oligonucleotides to yield pET28a/His-iBoNT/E (E213Q): E213Q Primer Pair, sense oligonucleotide, 5′-GCTGACTTTGATGCATCAACTGATCCATAGCTTGCACGGCCTG-3′ (SEQ ID NO: 113) and antisense oligonucleotide, 5′-CAGGCCGTGCAAGCTATGGATCAGTTGATGCATCAAAGTCAGC-3′ (SEQ ID NO: 114). The amino acid numbering corresponds to native sequence lacking an amino-terminal polyhistidine tag. The nucleotides that were changed to correct H216Y are shown in bold and nucleotides that were mutated to produce E213Q are shown in bold and underlined. The nucleic acid molecule of SEQ ID NO: 119 encodes a iBoNT/E (E213Q).

### Example 5

### Expression of pET28a/His-iBoNT/E (E213Q)

The following example illustrates a procedure useful for expressing a BoNT/E from an expression construct disclosed in the present specification. An pET28a/His-iBoNT/E (E213Q) expression construct was introduced into chemically competent E. coli BL21 (DE3) cells (Invitrogen, Inc, Carlsbad, Calif.) using a heat-shock transformation protocol. The heat-shock reaction was plated onto 1.5% Luria-Bertani agar plates (pH 7.0) containing 50 μg/mL of Kanamycin and placed in a 37° C. incubator for overnight growth. Kanamycin-resistant colonies of transformed E. coli containing pET28a/His-iBoNT/E (E213Q) were used to inoculate baffled flask containing 3.0 mL of PA-0.5G media containing 50 μg/mL of Kanamycin which was then placed in a 37° C. incubator, shaking at 250 rpm, for overnight growth. The resulting overnight starter culture was in turn used to inoculate a 3 L baffled flask containing ZYP-5052 autoinducing media containing 50 μg/mL of Kanamycin at a dilution of 1:1000. Culture volumes ranged from about 600 mL (20% flask volume) to about 750 mL (25% flask volume). These cultures were grown in a 37° C. incubator shaking at 250 rpm for approximately 5.5 hours until mid-log phase was reached (OD₆₀₀ of about 0.6-0.8). Cultures were then transferred to a 16° C. incubator shaking at 250 rpm for overnight expression. Cells were harvested by centrifugation (4,000 rpm at 4° C. for 20-30 minutes) and used immediately, or stored dry at −80° C. until needed.

### Example 6

### Purification and Quantification of His-iBoNT/E (E213Q)

The following example illustrates methods useful for purification and quantification of BoNT/E disclosed in the present specification. For immobilized metal affinity chromatography (IMAC) protein purification, E. coli BL21 (DE3) cell pellets used to express His-iBoNT/E (E213Q), as described in Example 5, were resuspended in Column Binding Buffer (25 mM N-(2-hydroxyethyl)piperazine-N′-(2-ethanesulfonic acid) (HEPES), pH 7.8; 500 mM sodium chloride; 10 mM imidazole; 2× Protease Inhibitor Cocktail Set III (EMD Biosciences-Calbiochem, San Diego Calif.); 5 units/mL of Benzonase (EMD Biosciences-Novagen, Madison, Wis.); 0.1% (v/v) TRITON-X® 100, 4-octylphenol polyethoxylate; 10% (v/v) glycerol), and then transferred to a cold Oakridge centrifuge tube. The cell suspension was sonicated on ice (10-12 pulses of 10 seconds at 40% amplitude with 60 seconds cooling intervals on a Branson Digital Sonifier) in order to lyse the cells and release the His-iBoNT/E, and then centrifuged (16,000 rpm at 4° C. for 20 minutes) to clarify the lysate. An immobilized metal affinity chromatography column was prepared using a 20 mL Econo-Pac column support (Bio-Rad Laboratories, Hercules, Calif.) packed with 2.5-5.0 mL of TALON™ SuperFlow Co²⁺ affinity resin (BD Biosciences-Clontech, Palo Alto, Calif.), which was then equilibrated by rinsing with 5 column volumes of deionized, distilled water, followed by 5 column volumes of Column Binding Buffer. The clarified lysate was applied slowly to the equilibrated column by gravity flow (approximately 0.25-0.3 mL/minute). The column was then washed with 5 column volumes of Column Wash Buffer (N-(2-hydroxyethyl)piperazine-N′-(2-ethanesulfonic acid) (HEPES), pH 7.8; 500 mM sodium chloride; 10 mM imidazole; 0.1% (v/v) TRITON-X® 100, 4-octylphenol polyethoxylate; 10% (v/v) glycerol). His-iBoNT/E was eluted with 20-30 mL of Column Elution Buffer (25 mM N-(2-hydroxyethyl)piperazine-N′-(2-ethanesulfonic acid) (HEPES), pH 7.8; 500 mM sodium chloride; 500 mM imidazole; 0.1% (v/v) TRITON-X® 100, 4-octylphenol polyethoxylate; 10% (v/v) glycerol) and collected in approximately twelve 1 mL fractions. The amount of His-iBoNT/E (E213Q) peptide contained in each elution fraction was determined by a Bradford dye assay. In this procedure, 20 μL aliquots of each 1.0 mL fraction was combined with 200 μL of Bio-Rad Protein Reagent (Bio-Rad Laboratories, Hercules, Calif.), diluted 1 to 4 with deionized, distilled water, and then the intensity of the colorimetric signal was measured using a spectrophotometer. The five fractions with the strongest signal were considered the elution peak and pooled. Total protein yield was determined by estimating the total protein concentration of the pooled peak elution fractions using bovine gamma globulin as a standard (Bio-Rad Laboratories, Hercules, Calif.).

For purification of a BoNT/E using a FPLC desalting column, a HIPREP™ 26/10 size exclusion column (Amersham Biosciences, Piscataway, N.J.) was pre-equilibrated with 80 mL of 4° C. Column Buffer (50 mM sodium phosphate, pH 6.5). After the column was equilibrated, a His-iBoNT/E (E213Q) sample was applied to the size exclusion column with an isocratic mobile phase of 4° C. Column Buffer and at a flow rate of 10 mL/minute using a BioLogic DuoFlow chromatography system (Bio-Rad Laboratories, Hercules, Calif.). The desalted His-iBoNT/E (E213Q) sample was collected as a single fraction of approximately 7-12 mL.

For purification of a BoNT/E using a FPLC ion exchange column, a His-iBoNT/E (E213Q) sample that had been desalted following elution from an IMAC column was applied to a 1 mL UNO-S1™ cation exchange column (Bio-Rad Laboratories, Hercules, Calif.) using a BioLogic DuoFlow chromatography system (Bio-Rad Laboratories, Hercules, Calif.). The sample was applied to the column in 4° C. Column Buffer (50 mM sodium phosphate, pH 6.5) and eluted by linear gradient with 4° C. Elution Buffer (50 mM sodium phosphate, 1 M sodium chloride, pH 6.5) as follows: step 1, 5.0 mL of 5% Elution Buffer at a flow rate of 1 mL/minute; step 2, 20.0 mL of 5-30% Elution Buffer at a flow rate of 1 mL/minute; step 3, 2.0 mL of 50% Elution Buffer at a flow rate of 1.0 mL/minute; step 4, 4.0 mL of 100% Elution Buffer at a flow rate of 1.0 mL/minute; and step 5, 5.0 mL of 0% Elution Buffer at a flow rate of 1.0 mL/minute. Elution of peptides from the column was monitored at 280, 260, and 214 nm, and peaks absorbing above a minimum threshold (0.01 au) at 280 nm were collected. Most of the His-iBoNT/E eluted at a sodium chloride concentration of approximately 100 to 200 mM. Average total yields of His-iBoNT/E (E213Q) were approximately 5-12 mg/L as determined by a Bradford assay.

Expression of the His-iBoNT/E (E213Q) was analyzed by polyacrylamide gel electrophoresis. Samples purified using the procedure described above were added to 2×LDS Sample Buffer (Invitrogen, Inc, Carlsbad, Calif.) and peptides separated by MOPS polyacrylamide gel electrophoresis using NUPAGE® Novex 4-12% Bis-Tris precast polyacrylamide gels (Invitrogen, Inc, Carlsbad, Calif.) under denaturing, reducing conditions. Gels were stained with SYPRO® Ruby (Bio-Rad Laboratories, Hercules, Calif.) and the separated peptides imaged using a Fluor-S MAX Multilmager (Bio-Rad Laboratories, Hercules, Calif.) for quantification of peptide expression levels. The size and amount of the His-iBoNT/E (E213Q) was determined by comparison to MagicMark™ protein molecular weight standards (Invitrogen, Inc, Carlsbad, Calif.). The gels revealed what appeared to be a full-length His-iBoNT/E (E213Q).

Expression of the His-iBoNT/E (E213Q) was also analyzed by Western blot analysis. Protein samples purified using the procedure described above were added to 2×LDS Sample Buffer (Invitrogen, Inc, Carlsbad, Calif.) and separated by MOPS polyacrylamide gel electrophoresis using NUPAGE® Novex 4-12% Bis-Tris precast polyacrylamide gels (Invitrogen, Inc, Carlsbad, Calif.) under denaturing, reducing conditions. Separated peptides were transferred from the gel onto polyvinylidene fluoride (PVDF) membranes (Invitrogen, Inc, Carlsbad, Calif.) by Western blotting using a TRANS-BLOT® SD semi-dry electrophoretic transfer cell apparatus (Bio-Rad Laboratories, Hercules, Calif.). PVDF membranes were blocked by incubating at room temperature for 2 hours in a solution containing 25 mM Tris-Buffered Saline (25 mM 2-amino-2-hydroxymethyl-1,3-propanediol hydrochloric acid (Tris-HCl)(pH 7.4), 137 mM sodium chloride, 2.7 mM potassium chloride), 0.1% TWEEN-20®, polyoxyethylene (20) sorbitan monolaureate, 2% bovine serum albumin, 5% nonfat dry milk. Blocked membranes were incubated at 4° C. for overnight in Tris-Buffered Saline TWEEN-20® (25 mM Tris-Buffered Saline, 0.1% TWEEN-20®, polyoxyethylene (20) sorbitan monolaureate) containing one of the following primary antibodies as a probe: a 1:50,000 dilution of rabbit polyclonal anti-BoNT/E antiserum (Metabiologics, Inc., Madison, Wis.); a 1:10,000 dilution of rabbit polyclonal anti-LC/E 3a antiserum (Allergan, Inc., generated under contract with Zymed Laboratories Inc., South San Francisco, Calif.); a 1:10,000 dilution of rabbit polyclonal anti-H_{c}/E 12 antiserum (Allergan, Inc., generated under contract with Zymed Laboratories Inc., South San Francisco, Calif.); or a 1:10,000 dilution of rabbit polyclonal anti-polyhistidine antiserum (Abcam Inc., Cambridge, Mass.). Primary antibody probed blots were washed three times for 15 minutes each time in Tris-Buffered Saline TWEEN-20®. Washed membranes were incubated at room temperature for 2 hours in Tris-Buffered Saline TWEEN-20® containing a 1:20,000 dilution of goat polyclonal anti-rabbit immunoglobulin G, heavy and light chains (IgG, H+L) antibody conjugated to horseradish peroxidase (HRP; Pierce Biotechnology, Inc., Rockford, Ill.) as a secondary antibody. Secondary antibody-probed blots were washed three times for 15 minutes each time in Tris-Buffered Saline TWEEN-20®. Signal detection of the labeled His-iBoNT/E (E213Q) was visualized using the ECL Plus™ Western Blot Detection System (Amersham Biosciences, Piscataway, N.J.) and imaged with a Typhoon 9410 Variable Mode Imager (Amersham Biosciences, Piscataway, N.J.) for quantification of His-iBoNT/E (E213Q) expression levels.

### Example 7

### Construction of pET28a/His-BoNT/E

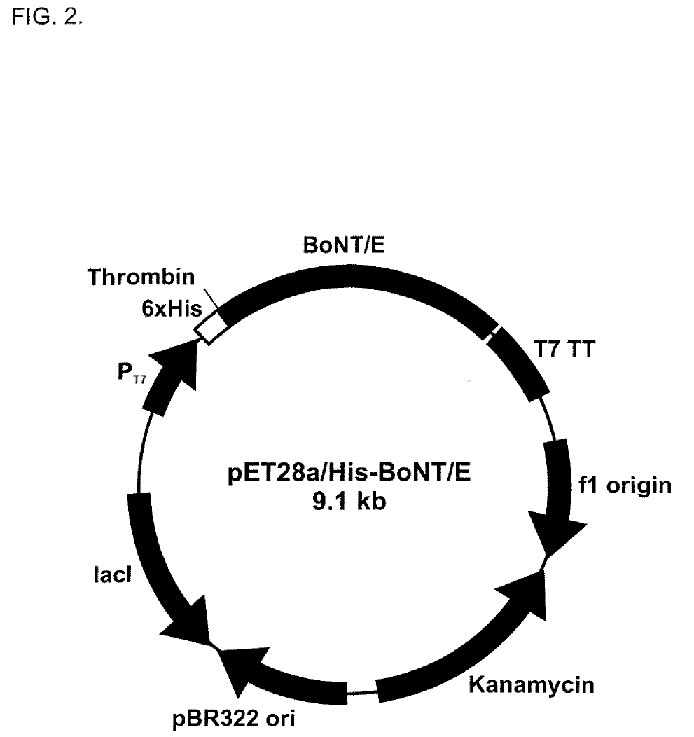

A plasmid comprising a modified open reading frame encoding an active BoNT/E (FIG. 2), was prepared by in vitro site-directed mutagenesis of pET28a/His-iBoNT/E (E213Q). Correction of the inactivating E213Q mutation was accomplished in a single site-directed mutagenesis step using the procedure described in Example 4 and the following two oligonucleotides to yield pET28a/His-BoNT/E: Q213E Primer Pair, sense oligonucleotide, 5′-GCTGACTTTGATGCATGAACTGATCCATAGCTTGCACG GCCTG-3′ (SEQ ID NO: 115) and antisense oligonucleotide, 5′-CAGGCCGTGCAAGCTATGGATCAGTT CATGCATCAAAGTCAGC-3′ (SEQ ID NO: 116). The amino acid numbering corresponds to native sequence lacking an amino-terminal polyhistidine tag. The nucleotides that were changed to correct E213Q are shown in bold and underlined. This mutagenesis resulted in the modified open reading frame of SEQ ID NO: 122 encoding the active His-BoNT/E of SEQ ID NO: 123.

Activity was identified by proteolytic cleavage of a GFP-SNAP25 substrate using a GFP-SNAP25 Fluorescence Release Assay, see, e.g., Lance E. Steward et al., GFP-SNAP25 Fluorescence Release Assay for Botulinum Neurotoxin Protease Activity, U.S. Patent Publication No. 2005/0100973 (May 12, 2005). Candidate pET28a/His-BoNT/E expression constructs were transformed into chemically competent E. coli BL21 (DE3) cells (Invitrogen, Inc, Carlsbad, Calif.) using a heat-shock method, plated onto 1.5% Luria-Bertani agar plates (pH 7.0) containing 50 μg/mL of Kanamycin, and placed in a 37° C. incubator for overnight growth. Kanamycin-resistant colonies containing the pET28a/His-BoNT/E candidates were used to inoculate 1 mL cultures of ZYP-5052 autoinducing media containing 50 μg/mL of Kanamycin in Eppendorf Lid-Bac tubes fitted with membrane lids. The cultures were incubated in a thermomixer (1,400 rpm at 37° C.) located in a biosafety cabinet until turbid (approximately 7-8 hours). The temperature was then reduced to 22° C. and the cultures incubated for approximately 16 hours. The cells were collected by centrifugation (6,000×g at 4° C. for 30 minutes), decanted and frozen briefly at −80° C. to improve lysis. The cell pellets were defrosted on ice, each was resuspended in 350 μL of BugBuster® lysis solution (EMD Biosciences-Novagen, Madison, Wis.) containing 25 units/mL of benzonase nuclease (EMD Biosciences-Novagen, Madison, Wis.), 1 KU/mL rLysozyme (EMD Biosciences-Novagen, Madison, Wis.) and 2× Protease Inhibitor Cocktail III (EMD Biosciences-Novagen, Madison, Wis.) and the mixtures were incubated for 30 minutes at 22° C., 400 rpm in the thermomixer. The lysates were clarified by centrifugation (36,000×g at 4° C. for 15 minutes) and the supernatant solutions transferred to low-retention microcentrifuge tubes and placed on ice.

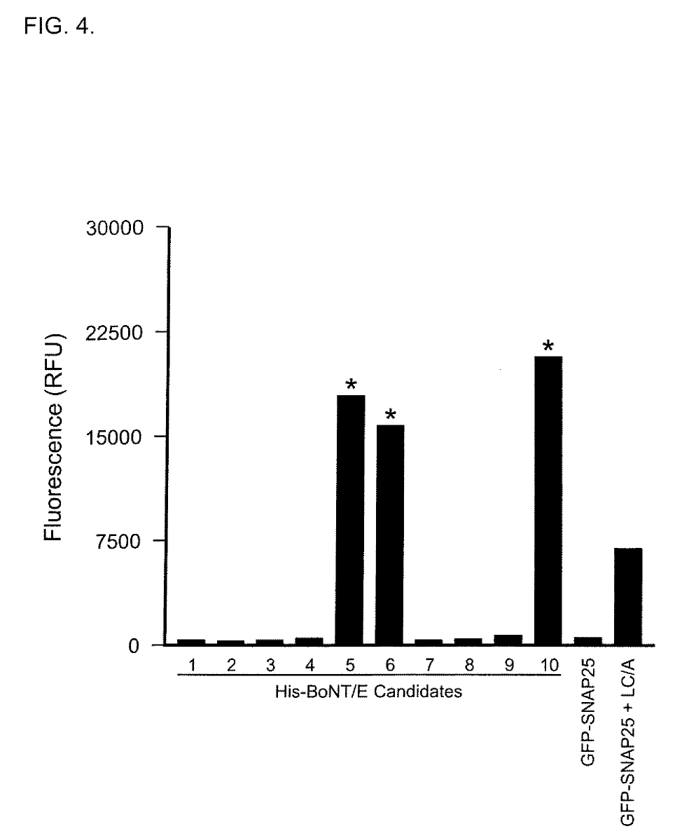

Activity of His-BoNT/E candidates was identified by proteolytic cleavage of a GFP-SNAP25 substrate. Each assay reaction contained 25 μL of 2× Toxin Reaction Buffer (100 mM N-(2-hydroxyethyl)piperazine-N′-(2-ethanesulfonic acid) (HEPES), pH 7.4; 20 μM zinc chloride; 20 mM dithiothreitol; 0.2% (v/v) TWEEN-20®, polyoxyethylene (20) sorbitan monolaureate), 10 μL of clarified lysate, and 15 μL of 50 μM GFP-SNAP25₍₁₃₄₋₂₀₆₎ substrate. The control reactions contained 10 μL of either water or 0.2 μg/mL of LC/A in lieu of lysate. The reactions were assembled in triplicate, incubated at 37° C. for 1 hour and then quenched with 20 μL of 8 M guanidine hydrochloride. The quenched reactions were transferred to filter-plate wells containing 75 μL of TALON™ SuperFlow Co²⁺ affinity resin (BD Biosciences-Clontech, Palo Alto, Calif.) that had been conditioned by rinsing with 200 μL of deionized, distilled water and 200 μL of Assay Rinse Buffer (50 mM N-(2-hydroxyethyl)piperazine-N′-(2-ethanesulfonic acid) (HEPES), pH 7.4). Following 15 minutes incubation on the resin, the reaction solutions were eluted by vacuum filtration, collected in a black 96-well plate, passed over the resin beds twice more and collected after the final pass. Each resin bed was then rinsed with 210 μL of Assay Rinse Buffer which was eluted into the plate containing the reaction solutions. The fluorescence of the eluant reaction solutions was measured with a SpectraMax Gemini XS spectrophotometer (Molecular Devices, λ_{EX} 474 nm; λ_{Em} 509 nm; 495 nm cutoff filter). The control reactions contained 10 μL of either water or 0.2 μg/mL of LC/A in lieu of lysate. Positive His-BoNT/E candidates showed significant protease activity (see FIG. 4).

TABLE 5
Activities of Native and Recombinant BoNT/E expressed as
ip LD₅₀ (ng/kg)

| BoNT/E | Single Chain | Dichain | Fold Activation |
|---|---|---|---|
| BTX-516 | 126 | 4.3 | 29 |
| BTX-541 | 3066 | 2.9 | 1057 |
| BTX-565 | 226 | 2.0 | 113 |
| His-BoNT/E | 7880 | 140 | 56 |
| BoNT/E-His | 1821 | 2.7 | 674 |

### Example 8

### Comparison of his-BoNT/E Amounts Expressed from Modified and Unmodified Open Reading Frames

The amount of increased BoNT/E expressed from a modified open reading frame as compared to an unmodified open reading frame can be determined as follows. In separate reactions, a pET28a/His-BoNT/E expression construct comprising the modified open reading frame of SEQ ID NO: 4 and a pET28a/His-BoNT/E construct comprising the unmodified open reading frame of SEQ ID NO: 3 are introduced into chemically competent E. coli BL21 (DE3) cells (Invitrogen, Inc, Carlsbad, Calif.) using a heat-shock transformation protocol. The heat-shock reactions are plated onto 1.5% Luria-Bertani agar plates (pH 7.0) containing 50 μg/mL of Kanamycin and are placed in a 37° C. incubator for overnight growth. Kanamycin-resistant colonies of transformed E. coli containing pET28a/His-BoNT/E constructs from both expression construct are used to inoculate separate 15 mL tubes containing 3.0 mL Kanamycin-resistance selective PA-0.5G media that are then placed in a 37° C. incubator, shaking at 250 rpm, for overnight growth. Approximately 600 μL of the resulting overnight starter culture from each construct is used to inoculate a 3.0 L baffled flask containing 600 mL Kanamycin-resistance, ZYP-5052 autoinducing media. The inoculated cultures are grown in a 37° C. incubator shaking at 250 rpm for approximately 5.5 hours until mid-log phase is reached (OD₆₀₀ of about 0.6-0.8). The flasks are then transferred to a 16° C. incubator shaking at 250 rpm for overnight expression. Cells are harvested by centrifugation (4,000 rpm at 4° C. for 20-30 minutes).

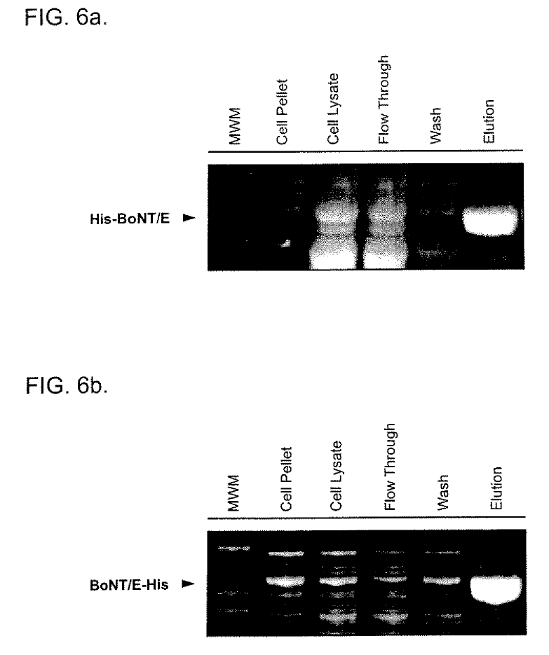

To analyze the His-BoNT/E expression levels obtained from both the modified and unmodified open reading frames, His-BoNT/E is purified using the IMAC procedure, as described in Example 6 (see FIG. 6a). Expression from each culture is evaluated by a Bradford dye assay, polyacrylamide gel electrophoresis and Western blot analysis (as described in Example 6) in order to determine whether the amounts of His-BoNT/E produced from the modified open reading frame of SEQ ID NO: 4 is greater when compared to the amount of His-BoNT/E expressed from the unmodified open reading frame of SEQ ID NO: 3. A four-fold increase in the amount of active His-iBoNT/E expressed from a modified open reading frame is anticipated. Average amounts of IMAC purified active His-iBoNT/E expressed from a modified open reading frame is expected to be approximately 9 mg/L, while IMAC purified active His-iBoNT/E expressed from a unmodified open reading frame in an otherwise identical nucleic acid molecule is expected to be approximately 3 mg/L.

### Example 9

### Construction of pET29a/iBoNT/E-His (E213Q)

To construct pET29a/iBoNT/E-His (E213Q), pET29a/iBoNT/E (E213Q) construct was made by digesting a pET28a/His-iBoNT/E (E213Q) construct with HindIII and NdeI at 37° C. for 2.5 hours to excise the iBoNT/E (E213Q) insert. The resulting restriction fragment was purified by the QIAquick Gel Extraction Kit (QIAGEN, Inc., Valencia, Calif.), and the fragment containing the open reading frame comprising SEQ ID NO: 119 was subcloned into the pET29a vector (EMD Biosciences-Novagen, Madison, Wis.) that had been digested with restriction endonucleases HindIII and NdeI. The fragment and vector were ligated using T4 DNA ligase protocol to yield pET29a/BoNT/E (E213Q). An aliquot from this ligation mixture was transformed by a standard heat-shock protocol into competent TOP10 cells (Invitrogen, Inc, Carlsbad, Calif.), plated onto 1.5% Luria-Bertani agar plates (pH 7.0) containing 50 μg/mL of Kanamycin, and placed in a 37° C. incubator for overnight growth. Candidate expression constructs were selected as Kanamycin-resistant colonies. Resistant colonies were used to inoculate 2 mL of Luria-Bertani media containing 50 μg/mL of Kanamycin that were then grown in a 37° C. incubator, shaking at 250 rpm, for 5 hours. The bacteria cells were harvested by microcentrifugation and the plasmid DNA was isolated using QIAGEN miniprep kits (QIAGEN, Inc., Valencia, Calif.). Candidate expression constructs were screened by restriction digestion with NdeI and HindIII to determine the presence of the correct insert fragment. Cultures containing the desired expression construct were used to inoculate 1 L baffled flasks containing 200 mL of Luria-Bertani media containing 50 μg/mL of Kanamycin and placed in a 37° C. incubator, shaking at 250 rpm, for overnight growth. Purified plasmid DNA corresponding to an expression construct was isolated using the QIAGEN Maxi-prep method (QIAGEN, Inc., Valencia, Calif.) and sequenced to verify that the correct expression construct was made (service contract with Sequetech Corp., Mountain View, Calif.). This cloning strategy yielded a prokaryotic expression construct encoding an iBoNT/E (E213Q).

A polyhistidine affinity binding peptide was fused in frame to iBoNT/E (E213Q) using a site-directed mutagenesis protocol that eliminated a stop codon immediately following the iBoNT/E (E213Q) open reading frame. A 50 μL reaction was assembled with the pET29a/iBoNT/E (E213Q) expression construct as a template, reagents included with the QuickChange® II XL Site-Directed Mutagenesis kit (Stratagene, La Jolla, Calif.) and the following two oligonucleotide primers: CtermHis Primer Pair, sense oligonucleotide, 5′-CCGCCAGCTTGTCGACTTTTTCTTGCCAGCCGTGC-3′ (SEQ ID NO: 120) and antisense oligonucleotide, 5′-GCACGGCTGGCAAGAAAAAGTCGACAAGCTGGCGG-3′ (SEQ ID NO: 121). A polymerase chain reaction (PCR) mix contained 5 μL of 10× Buffer, 1 μL of deoxyribonucleotides (dNTPs), 1 μL of PfuUltra™ High Fidelity DNA polymerase (2.5 units/μL), 125 ng of each primer, 50 ng of template DNA, and nuclease-free water to a final volume of 50 μL. The thermocycler conditions were: one cycle of 95° C. for 120 seconds; 20 cycles of 95° C. for 60 seconds, 55° C. for 30 seconds, and 72° C. for 20 minutes; one cycle of 72° C. for 9 minutes; and 10° C. to hold. Following thermocycling, 1 μL of DpnI restriction enzyme was added to the reaction and incubated for 2 hour at 37° C. to digest the template DNA. The reaction was purified by QIAquick kit (QIAGEN, Inc., Valencia, Calif.) and analysis by agarose gel electrophoresis showed that the reaction produced full-length plasmid. The mutagenesis products were transformed into chemically competent E. coli TOP10 cells (Invitrogen, Inc, Carlsbad, Calif.) using a heat shock method, plated on 1.5% Luria-Bertani agar plates (pH 7.0) containing 50 μg/mL of Kanamycin, and placed in a 37° C. incubator for overnight growth. Candidate constructs were isolated as Kanamycin-resistant colonies and analyzed using an alkaline lysis plasmid mini-preparation procedure to isolate the expression construct and restriction endonuclease digests to determine the presence of the insert. Deletion of the stop codon and confirmation of fusion protein construction were determined by sequence analysis (service contract with Sequetech Corp., Mountain View, Calif.) of candidate plasmid constructs. This cloning strategy yielded a prokaryotic expression construct encoding a BoNT/E-His (E213Q) containing an carboxy-terminal thrombin cleavable polyhistidine affinity binding peptide.

### Example 10

### Construction of pET29a/BoNT/E-His

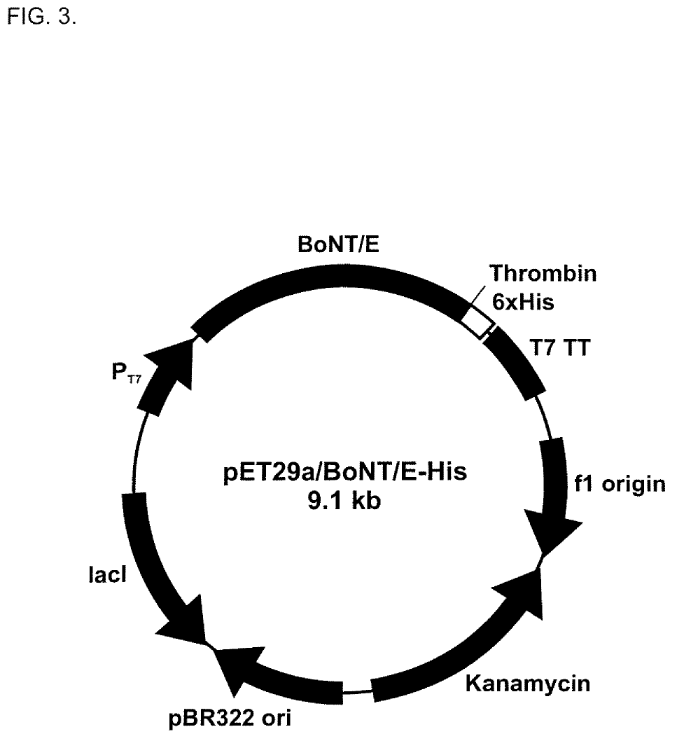

A plasmid comprising a modified open reading frame encoding an active BoNT/E (FIG. 3), was prepared by in vitro site-directed mutagenesis of pET29a/iBoNT/E-His (E213Q). Correction of the inactivating E213Q mutation was accomplished in a single site-directed mutagenesis step using the procedure described in Example 4 and the following two oligonucleotides to yield pET29a/BoNT/E-His: Q213E Primer Pair, sense oligonucleotide, 5′-GCTGACTTTGATGCATGAACTGATCCATAGCTTGCACG GCCTG-3′ (SEQ ID NO: 115) and antisense oligonucleotide, 5′-CAGGCCGTGCAAGCTATGGATCAGTT CATGCATCAAAGTCAGC-3′ (SEQ ID NO: 116). The nucleotides that were changed to correct E213Q are shown in bold and underlined. This mutagenesis resulted in the modified open reading frame of SEQ ID NO: 123 encoding the active His-BoNT/E of SEQ ID NO: 125.

Activity of BoNT/-His candidates was identified by proteolytic cleavage of a GFP-SNAP25 substrate using a GFP-SNAP25 Fluorescence Release Assay, see, e.g., Lance E. Steward et al., GFP-SNAP25 Fluorescence Release Assay for Botulinum Neurotoxin Protease Activity, U.S. Patent Publication No. 2005/0100973 (May 12, 2005). Candidate pET29a/BoNT/E-His expression constructs were transformed into chemically competent E. coli BL21 (DE3) cells (Invitrogen, Inc, Carlsbad, Calif.) using a heat-shock method, plated onto 1.5% Luria-Bertani agar plates (pH 7.0) containing 50 μg/mL of Kanamycin, and placed in a 37° C. incubator for overnight growth. Kanamycin-resistant colonies containing the pET29a/BoNT/E-His candidates were used to inoculate 1 mL cultures of ZYP-5052 autoinducing media containing 50 μg/mL of Kanamycin in Eppendorf Lid-Bac tubes fitted with membrane lids. The cultures were incubated in a thermomixer (1,400 rpm at 37° C.) located in a biosafety cabinet until turbid (approximately 7-8 hours). The temperature was then reduced to 22° C. and the cultures incubated for approximately 16 hours. The cells were collected by centrifugation (6,000×g at 4° C. for 30 minutes), decanted and frozen briefly at −80° C. to improve lysis. The cell pellets were defrosted on ice, each was resuspended in 350 μL of BugBuster® lysis solution (EMD Biosciences-Novagen, Madison, Wis.) containing 25 units/mL of benzonase nuclease (EMD Biosciences-Novagen, Madison, Wis.), 1 KU/mL rLysozyme (EMD Biosciences-Novagen, Madison, Wis.) and 2× Protease Inhibitor Cocktail III (EMD Biosciences-Novagen, Madison, Wis.) and the mixtures were incubated for 30 minutes at 22° C., 400 rpm in the thermomixer. The lysates were clarified by centrifugation (36,000×g at 4° C. for 15 minutes) and the supernatant solutions transferred to low-retention microcentrifuge tubes and placed on ice.

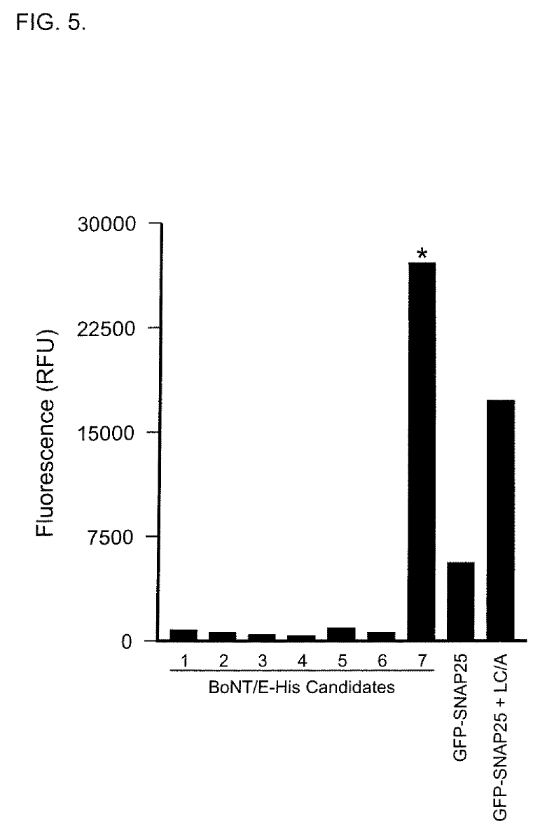

Activity of BoNT/E-His candidates was identified by proteolytic cleavage of a GFP-SNAP25 substrate. Each assay reaction contained 25 μL of 2× Toxin Reaction Buffer (100 mM N-(2-hydroxyethyl)piperazine-N′-(2-ethanesulfonic acid) (HEPES), pH 7.4; 20 μM zinc chloride; 20 mM dithiothreitol; 0.2% (v/v) TWEEN-20®, polyoxyethylene (20) sorbitan monolaureate), 10 μL of clarified lysate, and 15 μL of 50 μM GFP-SNAP25₍₁₃₄₋₂₀₆₎ substrate. The control reactions contained 10 μL of either water or 0.2 μg/mL of LC/A in lieu of lysate. The reactions were assembled in triplicate, incubated at 37° C. for 1 hour and then quenched with 20 μL of 8 M guanidine hydrochloride. The quenched reactions were transferred to filter-plate wells containing 75 μL of TALON™ SuperFlow Co²⁺ affinity resin (BD Biosciences-Clontech, Palo Alto, Calif.) that had been conditioned by rinsing with 200 μL of deionized, distilled water and 200 μL of Assay Rinse Buffer (50 mM N-(2-hydroxyethyl)piperazine-N′-(2-ethanesulfonic acid) (HEPES), pH 7.4). Following 15 minutes incubation on the resin, the reaction solutions were eluted by vacuum filtration, collected in a black 96-well plate, passed over the resin beds twice more and collected after the final pass. Each resin bed was then rinsed with 210 μL of Assay Rinse Buffer which was eluted into the plate containing the reaction solutions. The fluorescence of the eluant reaction solutions was measured with a SpectraMax Gemini XS spectrophotometer (Molecular Devices, λ_{EX} 474 nm; λ_{Em} 509 nm; 495 nm cutoff filter). The control reactions contained 10 μL of either water or 0.2 μg/mL of LC/A in lieu of lysate. Positive BoNT/E-His candidates showed significant protease activity (see FIG. 5).

### Example 11

### Expression of pET29a/BoNT/E-His

The following example illustrates a procedure useful for expressing a BoNT/E from an expression construct disclosed in the present specification. An pET29a/BoNT/E-His expression construct was introduced into chemically competent E. coli BL21 (DE3) cells (Invitrogen, Inc, Carlsbad, Calif.) using a heat-shock transformation protocol. The heat-shock reaction was plated onto 1.5% Luria-Bertani agar plates (pH 7.0) containing 50 pg/mL of Kanamycin and placed in a 37° C. incubator for overnight growth. Kanamycin-resistant colonies of transformed E. coli containing pET29a/BoNT/E-His were used to inoculate baffled flask containing 3.0 mL of PA-0.5G media containing 50 μg/mL of Kanamycin which was then placed in a 37° C. incubator, shaking at 250 rpm, for overnight growth. The resulting overnight starter culture was in turn used to inoculate a 3 L baffled flask containing ZYP-5052 autoinducing media containing 50 μg/mL of Kanamycin at a dilution of 1:1000. Culture volumes ranged from about 600 mL (20% flask volume) to about 750 mL (25% flask volume). These cultures were grown in a 37° C. incubator shaking at 250 rpm for approximately 5.5 hours until mid-log phase was reached (OD₆₀₀ of about 0.6-0.8). Cultures were then transferred to a 16° C. incubator shaking at 250 rpm for overnight expression. Cells were harvested by centrifugation (4,000 rpm at 4° C. for 20-30 minutes) and used immediately, or stored dry at −80° C. until needed.

### Example 12

### Purification and Quantification of BoNT/E-His

The following example illustrates methods useful for purification and quantification of BoNT/E disclosed in the present specification. For immobilized metal affinity chromatography (IMAC) protein purification, E. coli BL21 (DE3) cell pellets used to express BoNT/E-His, as described in Example 11, were resuspended in Column Binding Buffer (25 mM N-(2-hydroxyethyl)piperazine-N′-(2-ethanesulfonic acid) (HEPES), pH 7.8; 500 mM sodium chloride; 10 mM imidazole; 2× Protease Inhibitor Cocktail Set III (EMD Biosciences-Calbiochem, San Diego Calif.); 5 units/mL of Benzonase (EMD Biosciences-Novagen, Madison, Wis.); 0.1% (v/v) TRITON-X® 100, 4-octylphenol polyethoxylate; 10% (v/v) glycerol), and then transferred to a cold Oakridge centrifuge tube. The cell suspension was sonicated on ice (10-12 pulses of 10 seconds at 40% amplitude with 60 seconds cooling intervals on a Branson Digital Sonifier) in order to lyse the cells and release the BoNT/E-His, and then centrifuged (16,000 rpm at 4° C. for 20 minutes) to clarify the lysate. An immobilized metal affinity chromatography column was prepared using a 20 mL Econo-Pac column support (Bio-Rad Laboratories, Hercules, Calif.) packed with 2.5-5.0 mL of TALON™ SuperFlow Co²⁺ affinity resin (BD Biosciences-Clontech, Palo Alto, Calif.), which was then equilibrated by rinsing with 5 column volumes of deionized, distilled water, followed by 5 column volumes of Column Binding Buffer. The clarified lysate was applied slowly to the equilibrated column by gravity flow (approximately 0.25-0.3 mL/minute). The column was then washed with 5 column volumes of Column Wash Buffer (N-(2-hydroxyethyl)piperazine-N′-(2-ethanesulfonic acid) (HEPES), pH 7.8; 500 mM sodium chloride; 10 mM imidazole; 0.1% (v/v) TRITON-X® 100, 4-octylphenol polyethoxylate; 10% (v/v) glycerol). BoNT/E-His was eluted with 20-30 mL of Column Elution Buffer (25 mM N-(2-hydroxyethyl)piperazine-N′-(2-ethanesulfonic acid) (HEPES), pH 7.8; 500 mM sodium chloride; 500 mM imidazole; 0.1% (v/v) TRITON-X® 100, 4-octylphenol polyethoxylate; 10% (v/v) glycerol) and collected in approximately twelve 1 mL fractions. The amount of BoNT/E-His contained in each elution fraction was determined by a Bradford dye assay and the five fractions with the strongest signal were considered the elution peak and pooled (see FIG. 6b). Total protein yield was determined by estimating the total protein concentration of the pooled peak elution fractions using bovine gamma globulin as a standard (Bio-Rad Laboratories, Hercules, Calif.).

For purification of a BoNT/E using a FPLC desalting column, a HIPREP™ 26/10 size exclusion column (Amersham Biosciences, Piscataway, N.J.) was pre-equilibrated with 80 mL of 4° C. Column Buffer (50 mM sodium phosphate, pH 6.5). After the column was equilibrated, a BoNT/E-His sample was applied to the size exclusion column with an isocratic mobile phase of 4° C. Column Buffer and at a flow rate of 10 mL/minute using a BioLogic DuoFlow chromatography system (Bio-Rad Laboratories, Hercules, Calif.). The desalted BoNT/E-His sample was collected as a single fraction of approximately 7-12 mL.

For purification of a BoNT/E using a FPLC ion exchange column, a BoNT/E-His sample that had been desalted following elution from an IMAC column was applied to a 1 mL UNO-S1™ cation exchange column (Bio-Rad Laboratories, Hercules, Calif.) using a BioLogic DuoFlow chromatography system (Bio-Rad Laboratories, Hercules, Calif.). The sample was applied to the column in 4° C. Column Buffer (50 mM sodium phosphate, pH 6.5) and eluted by linear gradient with 4° C. Elution Buffer (50 mM sodium phosphate, 1 M sodium chloride, pH 6.5) as follows: step 1, 5.0 mL of 5% Elution Buffer at a flow rate of 1 mL/minute; step 2, 20.0 mL of 5-30% Elution Buffer at a flow rate of 1 mL/minute; step 3, 2.0 mL of 50% Elution Buffer at a flow rate of 1.0 mL/minute; step 4, 4.0 mL of 100% Elution Buffer at a flow rate of 1.0 mL/minute; and step 5, 5.0 mL of 0% Elution Buffer at a flow rate of 1.0 mL/minute. Elution of peptides from the column was monitored at 280, 260, and 214 nm, and peaks absorbing above a minimum threshold (0.01 au) at 280 nm were collected. Most of the BoNT/E-His eluted at a sodium chloride concentration of approximately 100 to 200 mM. Average total yields of BoNT/E-His were approximately 50-60 mg/L as determined by a Bradford assay.

Expression of BoNT/E-His was analyzed by polyacrylamide gel electrophoresis. Samples purified using the procedure described above were added to 2×LDS Sample Buffer (Invitrogen, Inc, Carlsbad, Calif.) and peptides separated by MOPS polyacrylamide gel electrophoresis using NUPAGE® Novex 4-12% Bis-Tris precast polyacrylamide gels (Invitrogen, Inc, Carlsbad, Calif.) under denaturing, reducing conditions. Gels were stained with SYPRO® Ruby (Bio-Rad Laboratories, Hercules, Calif.) and the separated peptides imaged using a Fluor-S MAX Multilmager (Bio-Rad Laboratories, Hercules, Calif.) for quantification of peptide expression levels. The size and amount of the BoNT/E-His was determined by comparison to MagicMark™ protein molecular weight standards (Invitrogen, Inc, Carlsbad, Calif.). The gels revealed what appeared to be a full-length BoNT/E-His.

Expression of BoNT/E-His was also analyzed by Western blot analysis. Protein samples purified using the procedure described above were added to 2×LDS Sample Buffer (Invitrogen, Inc, Carlsbad, Calif.) and separated by MOPS polyacrylamide gel electrophoresis using NUPAGE® Novex 4-12% Bis-Tris precast polyacrylamide gels (Invitrogen, Inc, Carlsbad, Calif.) under denaturing, reducing conditions. Separated peptides were transferred from the gel onto polyvinylidene fluoride (PVDF) membranes (Invitrogen, Inc, Carlsbad, Calif.) by Western blotting using a TRANS-BLOT® SD semi-dry electrophoretic transfer cell apparatus (Bio-Rad Laboratories, Hercules, Calif.). PVDF membranes were blocked by incubating at room temperature for 2 hours in a solution containing 25 mM Tris-Buffered Saline (25 mM 2-amino-2-hydroxymethyl-1,3-propanediol hydrochloric acid (Tris-HCl)(pH 7.4), 137 mM sodium chloride, 2.7 mM potassium chloride), 0.1% TWEEN-20®, polyoxyethylene (20) sorbitan monolaureate, 2% bovine serum albumin, 5% nonfat dry milk. Blocked membranes were incubated at 4° C. for overnight in Tris-Buffered Saline TWEEN-20® (25 mM Tris-Buffered Saline, 0.1% TWEEN-20®, polyoxyethylene (20) sorbitan monolaureate) containing one of the following primary antibodies as a probe: a 1:50,000 dilution of rabbit polyclonal anti-BoNT/E antiserum (Metabiologics, Inc., Madison, Wis.); a 1:10,000 dilution of rabbit polyclonal anti-LC/E 3a antiserum (Allergan, Inc., generated under contract with Zymed Laboratories Inc., South San Francisco, Calif.); a 1:10,000 dilution of rabbit polyclonal anti-H_{c}/E 12 antiserum (Allergan, Inc., generated under contract with Zymed Laboratories Inc., South San Francisco, Calif.); or a 1:10,000 dilution of rabbit polyclonal anti-polyhistidine antiserum (Abcam Inc., Cambridge, Mass.). Primary antibody probed blots were washed three times for 15 minutes each time in Tris-Buffered Saline TWEEN-20®. Washed membranes were incubated at room temperature for 2 hours in Tris-Buffered Saline TWEEN-20® containing a 1:20,000 dilution of goat polyclonal anti-rabbit immunoglobulin G, heavy and light chains (IgG, H+L) antibody conjugated to horseradish peroxidase (HRP; Pierce Biotechnology, Inc., Rockford, Ill.) as a secondary antibody. Secondary antibody-probed blots were washed three times for 15 minutes each time in Tris-Buffered Saline TWEEN-20®. Signal detection of the labeled BoNT/E-His was visualized using the ECL Plus™ Western Blot Detection System (Amersham Biosciences, Piscataway, N.J.) and imaged with a Typhoon 9410 Variable Mode Imager (Amersham Biosciences, Piscataway, N.J.) for quantification of peptide expression levels.

### Example 13

### Comparison of BoNT/E-His Amounts Expressed from Modified and Unmodified Open Reading Frames

The amount of increased BoNT/E expressed from a modified open reading frame as compared to an unmodified open reading frame can be determined as follows. In separate reactions, a pET29a/BoNT/E-His expression construct comprising the modified open reading frame of SEQ ID NO: 4 and a pET29a/BoNT/E-His construct comprising the unmodified open reading frame of SEQ ID NO: 3 are introduced into chemically competent E. coli BL21 (DE3) cells (Invitrogen, Inc, Carlsbad, Calif.) using a heat-shock transformation protocol. The heat-shock reactions are plated onto 1.5% Luria-Bertani agar plates (pH 7.0) containing 50 μg/mL of Kanamycin and are placed in a 37° C. incubator for overnight growth. Kanamycin-resistant colonies of transformed E. coli containing pET29a/BoNT/E-His constructs from both expression construct are used to inoculate separate 15 mL tubes containing 3.0 mL Kanamycin-resistance selective PA-0.5G media that are then placed in a 37° C. incubator, shaking at 250 rpm, for overnight growth. Approximately 600 μL of the resulting overnight starter culture from each construct are used to inoculate a 3.0 L baffled flask containing 600 mL Kanamycin-resistance, ZYP-5052 autoinducing media. The inoculated cultures are grown in a 37° C. incubator shaking at 250 rpm for approximately 5.5 hours until mid-log phase is reached (OD₆₀₀ of about 0.6-0.8). The flasks are then transferred to a 16° C. incubator shaking at 250 rpm for overnight expression. Cells are harvested by centrifugation (4,000 rpm at 4° C. for 20-30 minutes).

To analyze the BoNT/E-His expression amounts obtained from both the unmodified and modified open reading frames, BoNT/E-His is purified using the IMAC procedure (as described in Example 12). Expression from each culture is evaluated by a Bradford dye assay, polyacrylamide gel electrophoresis and Western blot analysis (as described in Example 12) in order to determine whether the amounts of His-BoNT/E produced from the modified open reading frame of SEQ ID NO: 4 is greater as compared to the amount of His-BoNT/E expressed from the unmodified open reading frame of SEQ ID NO: 3. An approximately 20-fold increase in the amount of active His-iBoNT/E expressed from a modified open reading frame is anticipated. Average amounts of IMAC purified active His-iBoNT/E expressed from a modified open reading frame is expected to be approximately 60 mg/L, while IMAC purified active BoNT/E-His expressed from a unmodified open reading frame in an otherwise identical nucleic acid molecule is expected to be approximately 3 mg/L.

### Example 14

### Construction and Expression of pRSET/BoNT/E-His

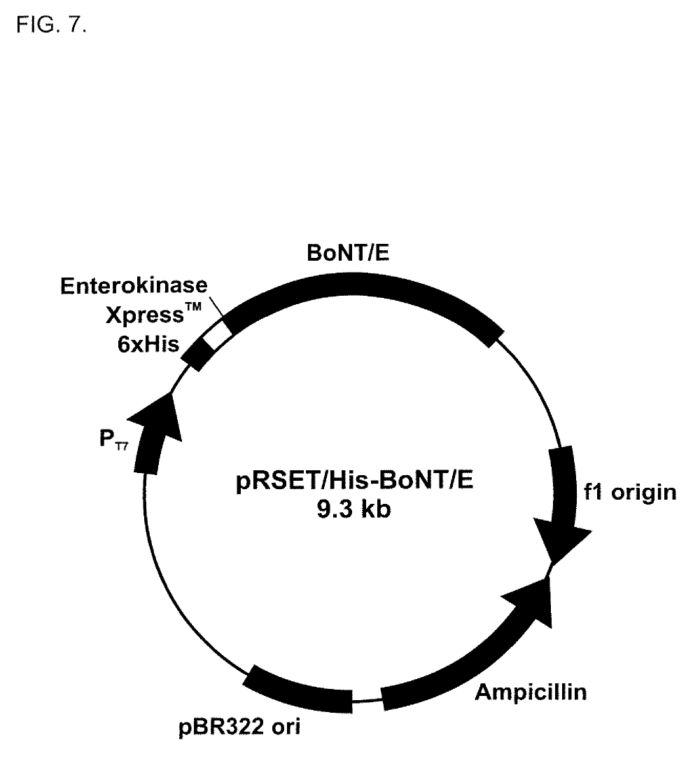

Restriction endonuclease sites suitable for cloning an operably linked nucleic acid molecule into a pRSET vector (Invitrogen, Inc, Carlsbad, Calif.) are incorporated into the 5′- and 3′ ends of modified open reading frame SEQ ID NO: 4. This nucleic acid molecule is synthesized and a pUCBHB1/BoNT/E construct obtained as described in Example 3. This construct is digested with restriction enzymes that 1) excise the insert containing the open reading frame of SEQ ID NO: 4 encoding an active BoNT/E; and 2) enable this insert to be operably-linked to a pRSET vector. This insert is subcloned using a T4 DNA ligase procedure into a pRSET vector that is digested with appropriate restriction endonucleases to yield pRSET/BoNT/E-His (FIG. 7). The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yields a prokaryotic expression construct encoding an active BoNT/E operably linked to carboxy-terminal polyhistidine binding peptide. A similar cloning strategy is used to make a pRSET construct containing the unmodified open reading frame of SEQ ID NO: 3 used as a control for expression levels, as well as, to produce pRSET expression constructs in which any one of the modified open reading frames of SEQ ID NO: 5 through SEQ ID NO: 34 is operably linked to a pRSET vector.

The amount of increased BoNT/E expression from a modified open reading frame is determined as follows. In separate reactions, a pRSET/BoNT/E-His expression construct comprising a modified open reading frame, such as, e.g., SEQ ID NO: 4 through SEQ ID NO: 34, and a pRSET/BoNT/E-His construct comprising an unmodified open reading frame, such as, e.g., SEQ ID NO: 3 are introduced into chemically competent bacterial cells suitable for expression of the pRET expression construct using a standard transformation protocol, such as, e.g., a heat-shock transformation protocol. The transformation reactions are plated onto 1.5% Luria-Bertani agar plates (pH 7.0) containing suitable antibiotics and placed in a 37° C. incubator for overnight growth. Antibiotic-resistant colonies of transformed cells containing pRSET/BoNT/E-His constructs from both nucleic acid molecules are used to inoculate separate 15 mL tubes containing 3.0 mL antibiotic-resistance selective PA-0.5G media that are then placed in a 37° C. incubator, shaking at 250 rpm, for overnight growth. Approximately 600 μL of the resulting overnight starter culture from each construct is used to inoculate a 3.0 L baffled flask containing 600 mL of a suitable antibiotic-resistance growth media. The inoculated cultures are grown in a 37° C. incubator shaking at 250 rpm for approximately 5.5 hours until mid-log phase is reached (OD₆₀₀ of about 0.6-0.8). The cultures are then induced by adding IPTG to a final concentration of 0.5-1.0 mM, and the cultures are transferred to a 16° C. incubator shaking at 250 rpm for overnight expression. Cells are harvested by centrifugation (4,000 rpm at 4° C. for 20-30 minutes).

To analyze the BoNT/E-His expression levels obtained from both the native and modified nucleic acid molecules, BoNT/E-His is purified using the IMAC procedure (as described in Example 12). Expression from each culture is evaluated by a Bradford dye assay, polyacrylamide gel electrophoresis and Western blot analysis using either anti-BoNT/E or anti-His antibodies (as described in Example 12) in order to determine whether the amounts of BoNT/E-His produced from the modified open reading frame was greater relative to the amount of BoNT/E-His expressed from the unmodified open reading frame of SEQ ID NO: 3.

### Example 15

### Construction and Expression of pPICZ A/BoNT/E-myc-His

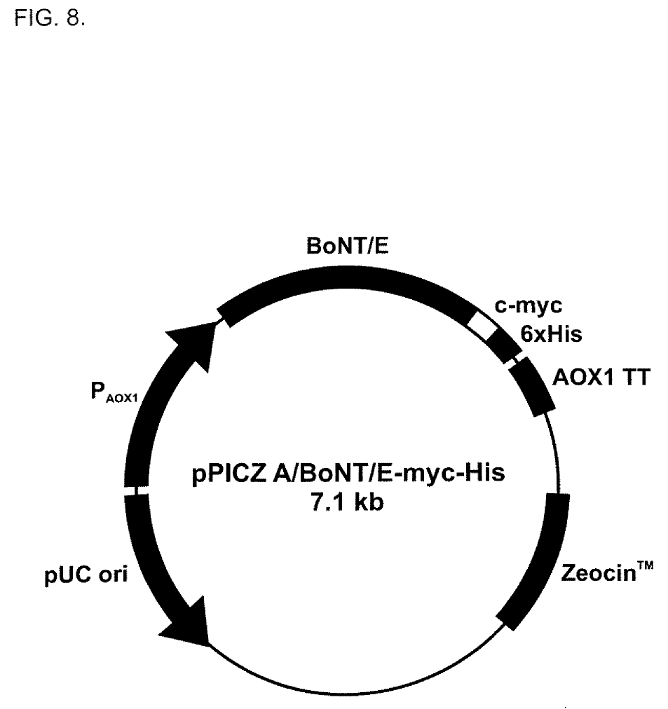

Restriction endonuclease sites suitable for cloning an operably linked nucleic acid molecule into a pPIC A vector (Invitrogen, Inc, Carlsbad, Calif.) are incorporated into the 5′- and 3′ ends of modified open reading frame SEQ ID NO: 37. This nucleic acid molecule is synthesized and a pUCBHB1/BoNT/E construct is obtained as described in Example 3. This construct is digested with restriction enzymes that 1) excise the insert containing the open reading frame of SEQ ID NO: 37 encoding an active BoNT/E; and 2) enable this insert to be operably-linked to a pPIC A vector. This insert is subcloned using a T4 DNA ligase procedure into a pPIC A vector that is digested with appropriate restriction endonucleases to yield pPIC A/BoNT/E-myc-His (FIG. 8). The ligation mixture is transformed into chemically competent E. coli DH5α cells (Invitrogen, Inc, Carlsbad, Calif.) using a heat shock method, plated on 1.5% low salt Luria-Bertani agar plates (pH 7.5) containing 25 μg/mL of Zeocin™, and placed in a 37° C. incubator for overnight growth. Bacteria containing expression constructs are identified as Zeocin™ resistant colonies. Candidate constructs are isolated using an alkaline lysis plasmid mini-preparation procedure and analyzed by restriction endonuclease digest mapping to determine the presence and orientation of the insert. This cloning strategy yields a yeast expression construct encoding an active BoNT/E operably linked to carboxy-terminal c-myc and polyhistidine binding peptides. A similar cloning strategy is used to make a pPIC A expression construct containing the unmodified open reading frame of SEQ ID NO: 3 used as a control for expression levels, as well as, to produce pPIC A expression constructs in which any one of the modified open reading frames of SEQ ID NO: 35, SEQ ID NO: 36, SEQ ID NO: 38 through SEQ ID NO: 46 is operably linked to a pPIC A vector.

To construct a yeast cell line expressing an active BoNT/E, pPICZ A/BoNT/E-myc-His is digested with a suitable restriction endonuclease (i.e., SacI, PmeI or BstXI) and the resulting linearized expression construct is transformed into an appropriate P. pastoris Mut^{S} strain KM71H using an electroporation method. The transformation mixture is plated on 1.5% YPDS agar plates (pH 7.5) containing 100 μg/mL of Zeocin™ and placed in a 28-30° C. incubator for 1-3 days of growth. Selection of transformants integrating the pPICZ A/BoNT/E-myc-His at the 5′ AOX1 locus is determined by colony resistance to Zeocin™. A similar strategy is used to make a cell line containing a pPICZ A expression construct containing SEQ ID NO: 3 used as a control for expression levels. Cell lines integrating a pPICZ A/BoNT/E-myc-His construct is tested for BoNT/E-myc-His expression using a small-scale expression test. Isolated colonies from test cell lines that have integrated pPICZ A/BoNT/E-myc-His are used to inoculate 1.0 L baffled flasks containing 100 mL of MGYH media and grown at about 28-30° C. in a shaker incubator (250 rpm) until the culture reaches an OD₆₀₀=2-6 (approximately 16-18 hours). Cells are harvested by centrifugation (3,000×g at 22° C. for 5 minutes). To induce expression, the cell pellet is resuspended in 15 mL of MMH media and 100% methanol is added to a final concentration of 0.5%. Cultures are grown at about 28-30° C. in a shaker incubator (250 rpm) for six days. Additional 100% methanol is added to the culture every 24 hours to a final concentration of 0.5%. A 1.0 mL test aliquot is taken from the culture every 24 hours starting at time zero and ending at time 144 hours. Cells are harvested from the aliquots by microcentrifugation to pellet the cells and lysed using three freeze-thaw rounds consisting of −80° C. for 5 minutes, then 37° C. for 5 minutes. Lysis samples are added to 2×LDS Sample Buffer (Invitrogen, Inc, Carlsbad, Calif.) and expression from established cell lines is measured by Western blot analysis (as described in Example 12) using either anti-BoNT/E, anti-myc or anti-His antibodies in order to identify lines expressing increased amounts of BoNT/E-myc-His produced from SEQ ID NO: 37 relative to established cell lines expressing BoNT/E-myc-His from the SEQ ID NO: 3 control. The P. pastoris Mut^{S} KM71H cell line showing the highest expression level of BoNT/E-myc-His relative to the SEQ ID NO: 3 control is selected for large-scale expression using commercial fermentation procedures. Procedures for large-scale expression are as outlined above except the culture volume is approximately 2.5 L MGYH media grown in a 5 L BioFlo 3000 fermentor and concentrations of all reagents will be proportionally increased for this volume. For greater details on all procedures described in this example, see EasySelect™ Pichia Expression Kit, version G, A Manual of Methods for Expression of Recombinant Proteins Using pPICZ and pPICZα in Pichia pastoris, 122701, 25-0172 (Invitrogen, Inc, Carlsbad, Calif.).

### Example 16

### Construction and Expression of pMET/BoNT/E-V5-His

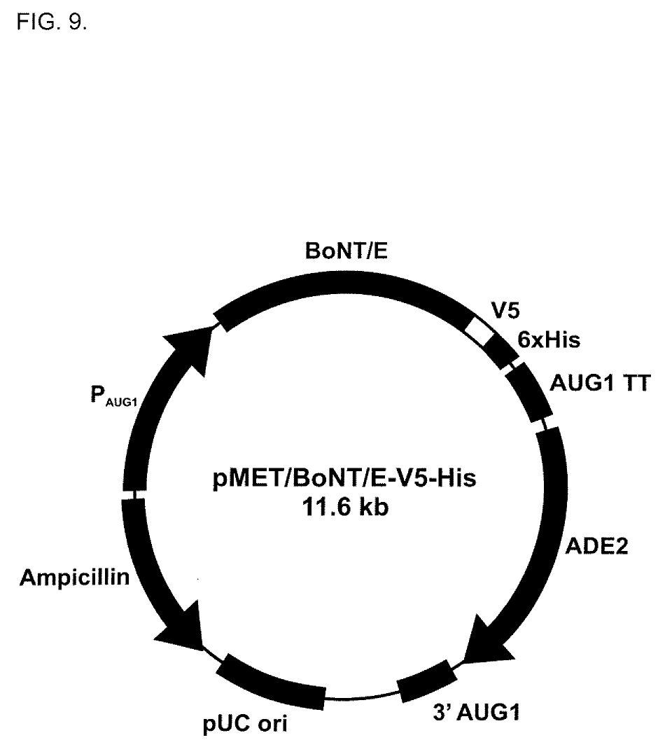

Restriction endonuclease sites suitable for cloning an operably linked nucleic acid molecule into a pMET vector (Invitrogen, Inc, Carlsbad, Calif.) are incorporated into the 5′- and 3′ ends of modified open reading frame SEQ ID NO: 37. This nucleic acid molecule is synthesized and a pUCBHB1/BoNT/E construct is obtained as described in Example 3. This construct is digested with restriction enzymes that 1) excise the insert containing the open reading frame of SEQ ID NO: 37 encoding an active BoNT/E; and 2) enable this insert to be operably-linked to a pMET vector. This insert is subcloned using a T4 DNA ligase procedure into a pMET vector that is digested with appropriate restriction endonucleases to yield pMET/BoNT/E-V5-His (FIG. 9). The ligation mixture is transformed into chemically competent E. coli DH5α cells (Invitrogen, Inc, Carlsbad, Calif.) using a heat shock method, plated on 1.5% low salt Luria-Bertani agar plates (pH 7.5) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yields a yeast expression construct encoding an active BoNT/E operably linked to carboxy-terminal V5 and polyhistidine binding peptides. A similar cloning strategy is used to make a pMET expression construct containing the unmodified open reading frame of SEQ ID NO: 3 used as a control for expression levels, as well as, to produce pMET expression constructs in which any one of the modified open reading frames of SEQ ID NO: 35, SEQ ID NO: 36, SEQ ID NO: 38 through SEQ ID NO: 46 is operably linked to a pMET vector.

To construct a yeast cell line expressing an active BoNT/E, pMET/BoNT/E-V5-His is digested with a suitable restriction endonuclease (i.e., ApaI, AscI, FseI, PacI, KpnI or PstI) and the resulting linearized expression construct is transformed into an appropriate P. methanolica Mut^{S} strain PMAD16 using an electroporation method. The transformation mixture is plated on 1.5% MD agar plates (pH 7.5) lacking adenine and grown in a 28-30° C. incubator for 3-4 days. Selection of transformants integrating the pMET/BoNT/E-V5-His is determined by colony growth on adenine-deficient media. A similar strategy is used to make a cell line containing a pMET expression construct containing SEQ ID NO: 3 used as a control for expression levels. Ade⁺ cell lines integrating a pMET/BoNT/E-V5-His construct are tested for BoNT/E-myc-His expression using a small-scale expression test. Isolated Ade⁺ colonies from test cell lines that have integrated pMET/BoNT/E-V5-His are used to inoculate 15 mL of BMDY media and cells are grown at about 28-30° C. in a shaker incubator (250 rpm) until the culture reaches an OD₆₀₀=2-10 (approximately 16-18 hours). Cells are harvested by centrifugation (1,500×g at 22° C. for 5 minutes). To induce expression, cell pellets are resuspended in 5 mL of BMMY media and cultures are grown at about 28-30° C. in a shaker incubator (250 rpm). After 24 hours, a 500 μL aliquot is removed, methanol is added to a final concentration of 0.5% and the cultures are grown at about 28-30° C. in a shaker incubator (250 rpm). A 500 μL aliquot is removed and additional methanol is added to a final concentration of 0.5% to the culture every 24 hours for 3-5 days. Harvested cells are centrifuged (1,500×g at 4° C. for 5 minutes), washed once in water and cell pellets stored at −80° C. until needed. To detect expression of the induced BoNT/E-V5-His, the cell pellets of each time point are lysed using an acid-washed glass bead method. Lysis samples are added to 2×LDS Sample Buffer (Invitrogen, Inc, Carlsbad, Calif.) and expression from established cell lines is measured by Western blot analysis (as described in Example 12) using either anti-BoNT/E, anti-V5 or anti-His antibodies in order to identify lines expressing increased amounts of BoNT/E-V5-His produced from SEQ ID NO: 37 relative to established cell lines expressing BoNT/E-V5-His from the SEQ ID NO: 3 control. The P. methanolica Mut^{s} PMAD16 cell line showing the highest expression level of BoNT/E-V5-His relative to the SEQ ID NO: 3 control is selected for large-scale expression using commercial fermentation procedures. Procedures for large-scale expression are as outlined above except the culture volume is approximately 2.5 L BMDY/BMMY media grown in a 5 L BioFlo 3000 fermentor and concentrations of all reagents will be proportionally increased for this volume. For greater details on all procedures described in this example, see P. methanolica Expression Kit, version C, A Manual of Methods for Expression of Recombinant Proteins in Pichia methanolica, 062101, 25-0288 (Invitrogen, Inc, Carlsbad, Calif.).

### Example 17

### Construction and Expression of pYES2/BoNT/E-V5-His

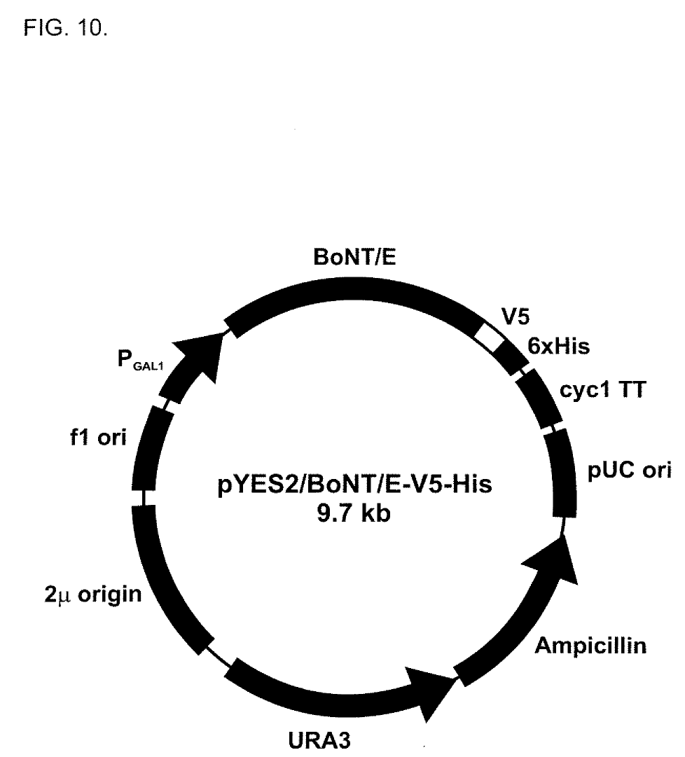

Restriction endonuclease sites suitable for cloning an operably linked nucleic acid molecule into a pYES2 vector (Invitrogen, Inc, Carlsbad, Calif.) are incorporated into the 5′ and 3′ ends of open reading frame SEQ ID NO: 40. This nucleic acid molecule is synthesized and a pUCBHB1/BoNT/E construct is obtained as described in Example 3. This construct is digested with restriction enzymes that 1) excise the insert containing the open reading frame of SEQ ID NO: 40 encoding an active BoNT/E; and 2) enable this insert to be operably-linked to a pYES2 vector. This insert is subcloned using a T4 DNA ligase procedure into a pYES2 vector that is digested with appropriate restriction endonucleases to yield pYES2/BoNT/E-V5-His (FIG. 10). The ligation mixture is transformed into chemically competent E. coli DH5α cells (Invitrogen, Inc, Carlsbad, Calif.) using a heat shock method, plated on 1.5% low salt Luria-Bertani agar plates (pH 7.5) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yields a yeast expression construct encoding an active BoNT/E operably linked to carboxy-terminal V5 and polyhistidine binding peptides. A similar cloning strategy is used to make a pYES2 expression construct containing the unmodified open reading frame of SEQ ID NO: 3 used as a control for expression levels, as well as, to produce pYES2 expression constructs in which any one of the modified open reading frames of SEQ ID NO: 35 through SEQ ID NO: 39 and SEQ ID NO: 41 through SEQ ID NO: 46 is operably linked to a pYES2 vector.

To construct a yeast cell line expressing an active BoNT/E, pYES2/BoNT/E-V5-His is transformed into competent S. cerevisiae strain INVSc1 using a Lithium-based transformation method. The transformation mixture is plated on 2% SC minimal media agar plates (pH 7.5) containing 2% glucose, that either have 0.01% uracil or lack uracil and placed in a 28-30° C. incubator for 1-3 days of growth. Selection of transformants containing pYES2/BoNT/E-V5-His is determined by colony growth only on plates containing uracil. A similar strategy is used to make cells containing a pYES2 expression construct containing SEQ ID NO: 3 used as a control for expression levels. Cells containing a pYES2/BoNT/E-V5-His construct are tested for BoNT/E-V5-His expression using a small-scale expression test. Isolated colonies from test cells containing pYES2/BoNT/E-V5-His are used to inoculate 50 mL tubes containing 15 mL of SC media containing 2% glucose and 0.01% uracil and grown overnight at about 28-30° C. in a shaker incubator (250 rpm). The OD₆₀₀ of overnight cultures are determined and aliquoted to obtain a cell concentration of OD₆₀₀ of 0.4 in a 50 mL volume. These aliquots are centrifuged (1,500×g at 22° C. for 5 minutes) and the resulting cell pellet resuspended in SC media containing 20% galactose and 10% raffinose. Cells are grown at about 28-30° C. in a shaker incubator (250 rpm) and 5 mL aliquots are taken at 0 hours, 4 hours, 8 hours, 12 hours, 16 hours and 24 hours and OD₆₀₀ concentrations are determined for each sample. Harvested cells are centrifuged (1,500×g at 4° C. for 5 minutes), washed once in water and cell pellets stored at −80° C. until needed. To detect expression of the induced BoNT/E-V5-His, the cell pellets of each time point are lysed using an acid-washed glass bead method. Lysis samples are added to 2×LDS Sample Buffer (Invitrogen, Inc, Carlsbad, Calif.) and expression from each time point is measured by Western blot analysis (as described in Example 12) using either anti-BoNT/E, anti-V5 or anti-His antibodies to identify the optimal induction time necessary to obtain maximal BoNT/E-V5-His expression. The induction conditions resulting in the highest expression level of BoNT/E-V5-His encoded by the modified open reading frame as compared to the unmodified open reading frame of SEQ ID NO: 3 control are selected for large-scale expression using commercial fermentation procedures. Procedures for large-scale expression are as outlined above except the culture volume is approximately 2.5 L SC media grown in a 5 L BioFlo 3000 fermentor and concentrations of all reagents will be proportionally increased for this volume. For greater details on all procedures described in this example, see pYES2/CT, pYES3/CT, and pYC2/CT Yeast Expression Vectors with C-terminal Tags and Auxotrophic Selection Markers, version E, 25-0304, Jan. 27, 2003 (Invitrogen, Inc, Carlsbad, Calif.).

### Example 18

### Construction and Expression of pFastBacHT/His-BoNT/E

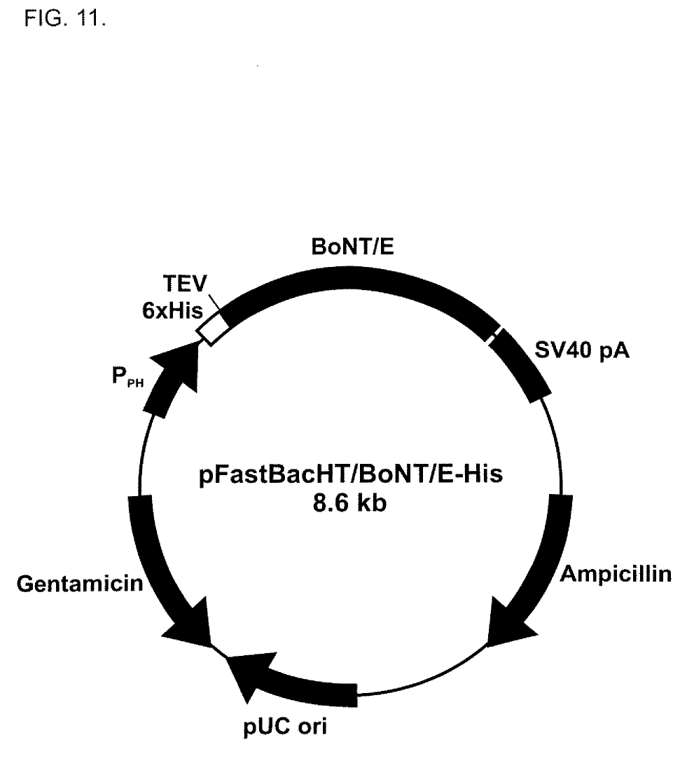

Restriction endonuclease sites suitable for cloning an operably linked nucleic acid molecule into a pFastBacHT vector (Invitrogen, Inc, Carlsbad, Calif.) are incorporated into the 5′- and 3′ ends of modified open reading frame SEQ ID NO: 61. This nucleic acid molecule is synthesized and a pUCBHB1/BoNT/E construct is obtained as described in Example 3. This construct is digested with restriction enzymes that 1) excise the insert containing the open reading frame of SEQ ID NO: 61 encoding an active BoNT/E; and 2) enable this insert to be operably-linked to a pFastBacHT vector. This insert is subcloned using a T4 DNA ligase procedure into a pFastBacHT vector that is digested with appropriate restriction endonucleases to yield pFastBacHT/His-BoNT/E (FIG. 11). The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yields a baculovirus transfer construct encoding an active BoNT/E operably linked to an amino-terminal, TEV cleavable, polyhistidine affinity binding peptide. A similar cloning strategy is used to make a pFastBacHT construct containing the unmodified open reading frame of SEQ ID NO: 3 used as a control for expression levels, as well as, to produce pFastBacHT expression constructs in which any one of the modified open reading frames of SEQ ID NO: 56, SEQ ID NO: 57, SEQ ID NO: 58, SEQ ID NO: 59 or SEQ ID NO: 60 is operably linked to a pFastBacHT vector.

To make a bacmid construct expressing an active BoNT/E, pFastBacHT/His-BoNT/E constructs are transformed by a heat shock method into MAX Efficiency® DH10Bac™ E. coli cells for transposition into a bacmid. The transformation mixture is plated on 1.5% Luria-Bertani agar plates (pH 7.0) containing 50 μg/mL of Kanamycin, 7 μg/mL of Gentamicin, 10 μg/mL of Tetracycline, 100 μg/mL of Bluo-gal and 40 μg/mL of IPTG and is grown for approximately 48 hours to isolate recombinant bacmid DNA. Candidate bacmid constructs are isolated as white colonies that are Kanamycin, Gentamicin and Tetracycline resistant. Candidate bacmid constructs are isolated using an alkaline lysis plasmid mini-preparation procedure and analyzed by restriction endonuclease digest mapping to determine the presence and orientation of the insert. A similar strategy is used to generate a recombinant baculoviral stock containing the unmodified open reading frame of SEQ ID NO: 3 construct. A P1 recombinant baculovirus stock is isolated by transfecting approximately 5×10⁵ Sf9 cells plated in a 35 mm tissue culture dish containing 2 mL of complete Sf-900 II SFM media with 50 units/mL of penicillin and 50 μg/mL of streptomycin, with 1.0 mL of transfection solution. The transfection solution is prepared by adding 800 μL of unsupplemented Grace's media to 200 μL of unsupplemented Grace's media, containing 1.0 μg of a purified bacmid His-BoNT/E construct and 6 μL of Cellfectin® Reagent preincubated for 30 minutes to allow formation of DNA:lipid complexes. Cells are incubated with this transfection solution for 5 hours in a 27° C. incubator, after which time this solution is replaced with 2.0 mL of complete Sf-900 II SFM media with 50 units/mL of penicillin and 50 μg/mL of streptomycin. Sf9 cells are grown for approximately 72 hours in a 27° C. incubator to allow for the release of virus into the medium. The virus is harvested by transferring the media from virally-infected insect cells to 15 mL snap-cap tubes and centrifuging tubes at 500×g for 5 minutes to remove debris. The clarified supernatant is transferred to fresh 15 mL snap-cap tubes and should contain approximately 1×10⁶ to 10⁷ plaque forming units (pfu) of baculovirus. This P1 viral stock is then amplified to generate a P2 recombinant baculovirus stock. About 2×10⁶ Sf9 cells are plated in a 35 mm culture dish containing 2 mL of Sf-900 II SFM media, supplemented with 50 units/mL of penicillin and 50 μg/mL of streptomycin, are inoculated with 400 μL of the P1 recombinant baculovirus stock (approximately 5×10⁶ pfu/ml) and incubated for approximately 48 hours in a 27° C. incubator. The virus is harvested by transferring the media to 15 mL snap-cap tubes and centrifuging tubes at 500×g for 5 minutes to remove debris. The clarified supernatant is transferred to fresh 15 mL snap-cap tubes and should contain approximately 1×10⁷ to 10⁸ pfu of baculovirus.

To express His-BoNT/E using a baculoviral expression system, about 2×10⁶ Sf9 cells are plated in a 35 mm culture dish containing 2 mL of Sf-900 II SFM media, supplemented with 50 units/mL of penicillin and 50 μg/mL of streptomycin, are inoculated with approximately 4 μL of the P1 recombinant baculovirus stock (approximately 5×10⁷ pfu/ml) and incubated for approximately 48 hours in a 27° C. incubator. Both media and cells are collected for BoNT/E-His expression. Media is harvested by transferring the media to 15 mL snap-cap tubes and centrifuging tubes at 500×g for 5 minutes to remove debris. Cells are harvested by rinsing cells once with 3.0 mL of 100 mM phosphate-buffered saline, pH 7.4 and lysing cells with a buffer containing 62.6 mM 2-amino-2-hydroxymethyl-1,3-propanediol hydrochloric acid (Tris-HCl), pH 6.8 and 2% sodium lauryl sulfate (SDS). Both media and cell samples are added to 2×LDS Sample Buffer (Invitrogen, Inc, Carlsbad, Calif.) and expression is measured by Western blot analysis (as described in Example 12) using either anti-BoNT/E or anti-His antibodies in order to identify P2 baculoviral stocks expressing increased amounts of His-BoNT/E produced from SEQ ID NO: 61 relative to stocks expressing His-BoNT/E from the SEQ ID NO: 3 control. For greater details on all procedures described in this example, see Bac-to-Bac® Baculovirus Expression System, version D, An Efficient Site-specific Transposition System to Generate Baculovirus for High-level Expression of Recombinant Proteins, 10359 (Invitrogen, Inc, Carlsbad, Calif.).

### Example 19

### Construction and Expression of pBACgus3/gp64-BoNT/E-His

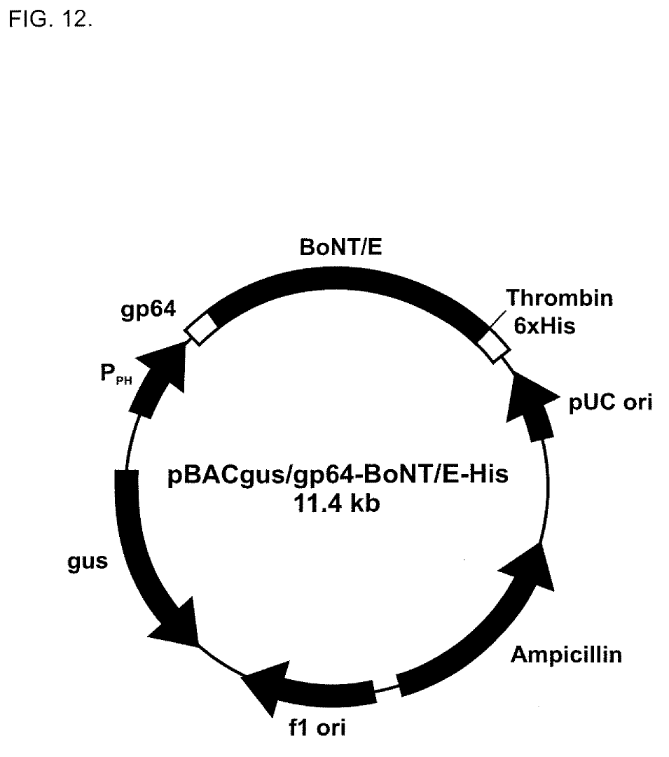

Restriction endonuclease sites suitable for cloning an operably linked nucleic acid molecule into a pBACgus3 vector (EMD Biosciences-Novagen, Madison, Wis.) are incorporated into the 5′- and 3′ ends of modified open reading frame SEQ ID NO: 61. This nucleic acid molecule is synthesized and a pUCBHB1/BoNT/E construct is obtained as described in Example 3. This construct is digested with restriction enzymes that 1) excise the insert containing the open reading frame of SEQ ID NO: 61 encoding an active BoNT/E; and 2) enable this insert to be operably-linked to a pBACgus3 vector. This insert is subcloned using a T4 DNA ligase procedure into a pBACgus3 vector that is digested with appropriate restriction endonucleases to yield pBACgus3/BoNT/E-His (FIG. 12). The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yields a baculovirus transfer construct encoding an active BoNT/E operably linked to an amino-terminal gp64 signal peptide and a carboxy-terminal, thrombin cleavable, polyhistidine affinity binding peptide. A similar cloning strategy is used to make a pBACgus3 construct containing the unmodified open reading frame of SEQ ID NO: 3 used as a control for expression levels, as well as, to produce pBACgus3 expression constructs in which any one of the modified open reading frames of SEQ ID NO: 56, SEQ ID NO: 57, SEQ ID NO: 58, SEQ ID NO: 59 or SEQ ID NO: 60 is operably linked to a pBACgus3 vector.

To express BoNT/E-His using a baculoviral expression system, about 2.5×10⁶ Sf9 cells are plated in four 60 mm culture dishes containing 2 mL of BACVECTOR® Insect media, a serum-free S19 insect cell growth media, (EMD Biosciences-Novagen, Madison, Wis.) and incubated for approximately 20 minutes in a 28° C. incubator. For each transfection, a 50 μL transfection solution is prepared in a 6 mL polystyrene tube by adding 25 μL of BACVECTOR® Insect media, a serum-free S19 insect cell growth media, containing 100 ng pBACgus3/gp64-BoNT/E-His and 500 ng TIowE transfer plasmid to 25 μL of diluted Insect GENEJUICE®, a cationic liposome based reagent, containing 5 μL Insect GENEJUICE®, a cationic liposome based reagent, (EMD Biosciences-Novagen, Madison, Wis.) and 20 μL nuclease-free water and this solution is incubated for approximately 15 minutes. After the 15 minute incubation, add 450 μL BACVECTOR® media, a serum-free S19 insect cell growth media, to the transfection solution and mix gently. Using this stock transfection solution as the 1/10 dilution make additional transfection solutions of 1/50, 1/250 and 1/1250 dilutions. Add 100 μL of a transfection solution to the Sf9 cells from one of the four 60 mm culture dishes, twice washed with antibiotic-free, serum-free BACVECTOR® Insect media, a serum-free S19 insect cell growth media, and incubate at 22° C. After one hour, add 6 mL of 1% BacPlaque agarose BACVECTOR® Insect media, a serum-free S19 insect cell growth media, containing 5% bovine serum albumin. After the agarose is solidified, add 2 mL BACVECTOR® Insect media, a serum-free S19 insect cell growth media, containing 5% bovine serum albumin to the transfected cells and transfer the cells to a 28° C. incubator for 3-5 days until plaques are visible. After 3-5 days post-transfection, plaques in the monolayer will be stained for R-glucuronidase reporter gene activity to test for the presence of recombinant virus plaques containing pBACgus3/BoNT/E-His by incubating the washed monolayer with 2 mL of BACVECTOR® Insect media, a serum-free S19 insect cell growth media, containing 30 μL of 20 mg/mL X-Gluc Solution (EMD Biosciences-Novagen, Madison, Wis.) for approximately 2 hours in a 28° C. incubator.

After identifying candidate recombinant virus plaques, several candidate virus plaques are eluted and plaque purified. To elute a recombinant virus, transfer a plug containing a recombinant virus plaque with a sterile Pasteur pipet to 1 mL BACVECTOR® Insect media, a serum-free S19 insect cell growth media, (EMD Biosciences-Novagen, Madison, Wis.) in a sterile screw-cap vial. Incubate the vial for approximately 2 hours at 22° C. or for approximately 16 hours at 4° C. For each recombinant virus plaque, 2.5×10⁵ Sf9 cells are plated in 35 mm culture dishes containing 2 mL of BACVECTOR® Insect media, a serum-free S19 insect cell growth media, (EMD Biosciences-Novagen, Madison, Wis.) and incubated for approximately 20 minutes in a 28° C. incubator. Remove the media and add 200 μL of eluted recombinant virus. After one hour, add 2 mL of 1% BacPlaque agarose-BACVECTOR® Insect media, a serum-free S19 insect cell growth media, containing 5% bovine serum albumin. After the agarose is solidified, add 1 mL BACVECTOR® Insect media, a serum-free S19 insect cell growth media, containing 5% bovine serum albumin to the transfected cells and transfer the cells to a 28° C. incubator for 3-5 days until plaques are visible. After 3-5 days post-transfection, plaques in the monolayer will be stained for β-glucuronidase reporter gene activity to test for the presence of recombinant virus plaques containing pBACgus3/BoNT/E-His by incubating the washed monolayer with 2 mL of BACVECTOR® Insect media, a serum-free S19 insect cell growth media, containing 30 μL of 20 mg/mL X-Gluc Solution (EMD Biosciences-Novagen, Madison, Wis.) for approximately 2 hours in a 28° C. incubator.

To prepare a seed stock of virus, elute a recombinant virus by transferring a plug containing a recombinant virus plaque with a sterile Pasteur pipet to 1 mL BACVECTOR® Insect media, a serum-free S19 insect cell growth media, (EMD Biosciences-Novagen, Madison, Wis.) in a sterile screw-cap vial. Incubate the vial for approximately 16 hours at 4° C. Approximately 5×10⁵ Sf9 cells are plated in T-25 flask containing 5 mL of BACVECTOR® Insect media, a serum-free S19 insect cell growth media, (EMD Biosciences-Novagen, Madison, Wis.) and are incubated for approximately 20 minutes in a 28° C. incubator. Remove the media and add 300 μL of eluted recombinant virus. After one hour, add 5 mL BACVECTOR® Insect media, a serum-free S19 insect cell growth media, containing 5% bovine serum albumin to the transfected cells and transfer the cells to a 28° C. incubator for 3-5 days until the majority of cells become unattached and unhealthy. The virus is harvested by transferring the media to 15 mL snap-cap tubes and centrifuging tubes at 1000×g for 5 minutes to remove debris. The clarified supernatant is transferred to fresh 15 mL snap-cap tubes and are stored at 4° C.

To prepare a high titer stock of virus, approximately 2×10⁷ Sf9 cells are plated in T-75 flask containing 10 mL of BACVECTOR® Insect media, a serum-free S19 insect cell growth media, (EMD Biosciences-Novagen, Madison, Wis.) and are incubated for approximately 20 minutes in a 28° C. incubator. Remove the media and add 500 μL of virus seed stock. After one hour, add 10 mL BACVECTOR® Insect media, a serum-free S19 insect cell growth media, containing 5% bovine serum albumin to the transfected cells and transfer the cells to a 28° C. incubator for 3-5 days until the majority of cells become unattached and unhealthy. The virus is harvested by transferring the media to 15 mL snap-cap tubes and centrifuging tubes at 1000×g for 5 minutes to remove debris. The clarified supernatant is transferred to fresh 15 mL snap-cap tubes and are stored at 4° C. High titer virus stocks should contain approximately 2×10⁸ to 3×10⁹ pfu of baculovirus.

To express gp64-BoNT/E-His using a baculoviral expression system, about 1.25×10⁸ Sf9 cells are seeded in a 1 L flask containing 250 mL of BACVECTOR® Insect media, a serum-free S19 insect cell growth media, and are grown in an orbital shaker (150 rpm) to a cell density of approximately 5×10⁸. The culture is inoculated with inoculated with approximately 2.5×10⁹ of high titer stock recombinant baculovirus and incubated for approximately 48 hours in a 28° C. orbital shaker (150 rpm). Media is harvested by transferring the media to tubes and centrifuging tubes at 500×g for 5 minutes to remove debris. Media samples are added to 2×LDS Sample Buffer (Invitrogen, Inc, Carlsbad, Calif.) and expression is measured by Western blot analysis (as described in Example 12) using either anti-BoNT/E or anti-His antibodies in order to identify baculoviral stocks expressing increased amounts of His-BoNT/E produced from SEQ ID NO: 61 relative to stocks expressing gp64-BoNT/E-His from the SEQ ID NO: 3 control. For greater details on all procedures described in this example, see BACVECTOR® Transfection Kits, TB216, revision A 1203, a baculovirus expression vector and a cationic liposome based reagent, (EMD Biosciences-Novagen, Madison, Wis.).

### Example 20

### Construction and Expression of pMT/BiP-BoNT/E-V5-His

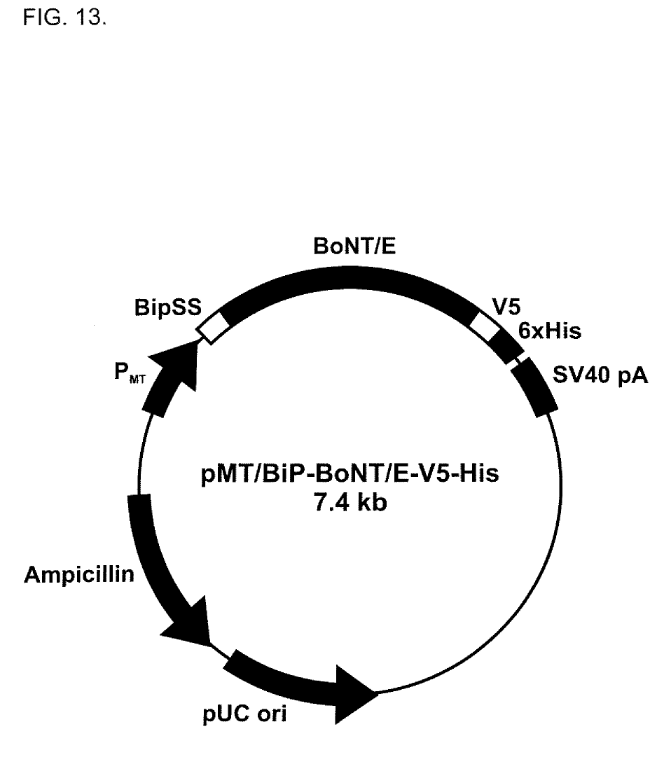

Restriction endonuclease sites suitable for cloning an operably linked nucleic acid molecule into a pMT vector (Invitrogen, Inc, Carlsbad, Calif.) are incorporated into the 5′- and 3′ ends of modified open reading frame SEQ ID NO: 58. This nucleic acid molecule is synthesized and a pUCBHB1/BoNT/E construct obtained as described in Example 3. This construct is digested with restriction enzymes that 1) excise the insert containing the open reading frame of SEQ ID NO: 58 encoding an active BoNT/E; and 2) enable this insert to be operably-linked to a pMT vector. This insert is subcloned using a T4 DNA ligase procedure into a pMT vector that is digested with appropriate restriction endonucleases to yield pMT/BiP-BoNT/E-V5-His (FIG. 13). The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy will yield an insect expression construct encoding an active BoNT/E operably linked to carboxy-terminal V5 and polyhistidine binding peptides. A similar cloning strategy is used to make a pMT construct containing the unmodified open reading frame of SEQ ID NO: 3 used as a control for expression levels, as well as, to produce pMT expression constructs in which any one of the modified open reading frames of SEQ ID NO: 56, SEQ ID NO: 57, SEQ ID NO: 59, SEQ ID NO: 60 or SEQ ID NO: 61 is operably linked to a pMT vector.

To transiently express active BoNT/E-V5-His in insect cells, about 3×10⁶ S2 cells are plated in a 35 mm tissue culture dish containing 3 mL of Schneider's Drosophila media and are grown in a 28° C. incubator until cells reach a density of approximately 9×10⁶ cells/ml (6-16 hours). A 600 μL transfection solution is prepared by adding 300 μL of 2×HEPES-Buffered Saline, pH 7.1 (50 mM N-(2-hydroxyethyl)piperazine-N′-(2-ethanesulfonic acid) (HEPES), pH 7.4; 1.5 mM sodium phosphate (monobasic); 280 mM sodium chloride) to 300 μL of 240 mM calcium chloride containing 19 μg of pMT/BiP-BoNT/E-V5-His and this solution is incubated for approximately 30 minutes. The transfection solution is added to S2 cells and the cells are incubated in a 28° C. incubator for approximately 16-24 hours. The transfection media is replaced with 3 mL of fresh Schneider's Drosophila media containing 500 μM copper sulfate to induce expression. Cells are incubated in a 28° C. incubator for an additional 48 hours. Media is harvested by transferring the media to 15 mL snap-cap tubes and centrifuging tubes at 500×g for 5 minutes to remove debris. Samples are added to 2×LDS Sample Buffer (Invitrogen, Inc, Carlsbad, Calif.) and expression is measured by Western blot analysis (as described in Example 12) using either anti-BoNT/E, anti-V5 or anti-His antibodies in order to identify pMT constructs expressing increased amounts of BiP-BoNT/E-V5-His produced from SEQ ID NO: 58 relative to constructs expressing BoNT/E-V5-His from the SEQ ID NO: 3 control.

To generate a stably-integrated insect cell line expressing active BoNT/E-V5-His, approximately 3×10⁶ S2 cells are plated in a 35 mm tissue culture dish containing 3 mL of Schneider's Drosophila media and grown in a 28° C. incubator until cells reach a density of about 9×10⁶ cells/ml (6-16 hours). A 600 μL transfection solution is prepared by adding 300 μL of 2×HEPES-Buffered Saline, pH 7.1 (50 mM N-(2-hydroxyethyl)piperazine-N′-(2-ethanesulfonic acid) (HEPES), pH 7.4; 1.5 mM sodium phosphate (monobasic); 280 mM sodium chloride) to 300 μL of 240 mM calcium chloride containing 19 μg of pMT/BiP-BoNT/E-V5-His and 1 μg of pCoHygro, and this solution is incubated for approximately 30 minutes. The transfection solution is added to S2 cells and incubate in a 28° C. incubator for approximately 16-24 hours. Transfection media is replaced with 3 mL of fresh Schneider's Drosophila media and the cells are incubated in a 28° C. incubator for approximately 48 hours. Media is replaced with 3 mL of fresh Schneider's Drosophila media containing approximately 500 μg/mL of hygromycin-B. Cells are incubated in a 28° C. incubator for approximately 3-4 weeks, and old media is replaced with fresh hygromycin-B selective media every 4 to 5 days. Once hygromycin-B-resistant colonies are established, resistant clones are replated to new 35 mm culture plates containing fresh Schneider's Drosophila media supplemented with approximately 500 μg/mL of hygromycin-B until these cells reach a density of about 6 to 20×10⁶ cells/mL. To test for expression of BoNT/E-V5-His from S2 cell lines that have stably-integrated a pMT/BiP-BoNT/E-V5-His, approximately 3×10⁶ S2 cells from each cell line are plated in a 35 mm tissue culture dish containing 3 mL of Schneider's Drosophila media and are grown in a 28° C. incubator until cells reach a density of about 9×10⁶ cells/ml (6-16 hours). Transfection media is replaced with 3 mL of fresh Schneider's Drosophila media containing 500 μM copper sulfate to induce expression. Cells are incubated in a 28° C. incubator for an additional 48 hours. Media is harvested by transferring the media to 15 mL snap-cap tubes and centrifuging tubes at 500×g for 5 minutes to remove debris. Samples are added to 2×LDS Sample Buffer (Invitrogen, Inc, Carlsbad, Calif.) and expression is measured by Western blot analysis (as described in Example 12) using either anti-BoNT/E, anti-V5 or anti-His antibodies in order to identify S2 cell lines expressing increased amounts of BoNT/E-V5-His produced from SEQ ID NO: 58 relative to cell lines expressing BoNT/E-V5-His from the SEQ ID NO: 3 control. The established S2 cell line showing the highest expression level of BoNT/E-V5-His relative to the SEQ ID NO: 3 control is selected for large-scale expression using 3 L spinner flasks. Procedures for large-scale expression are as outlined above except the culture volume is approximately 800-1000 mL of Schneider's Drosophila media and concentrations of all reagents are proportionally increased for this volume. For greater details on all procedures described in this example, see Drosophila Expression System, version H, For the Stable Expression and Purification of Heterologous Proteins in Schneider 2 Cells, 25-0191 (Invitrogen, Inc, Carlsbad, Calif.).

### Example 21

### Construction and Expression of pQBI25/BoNT/E-GFP

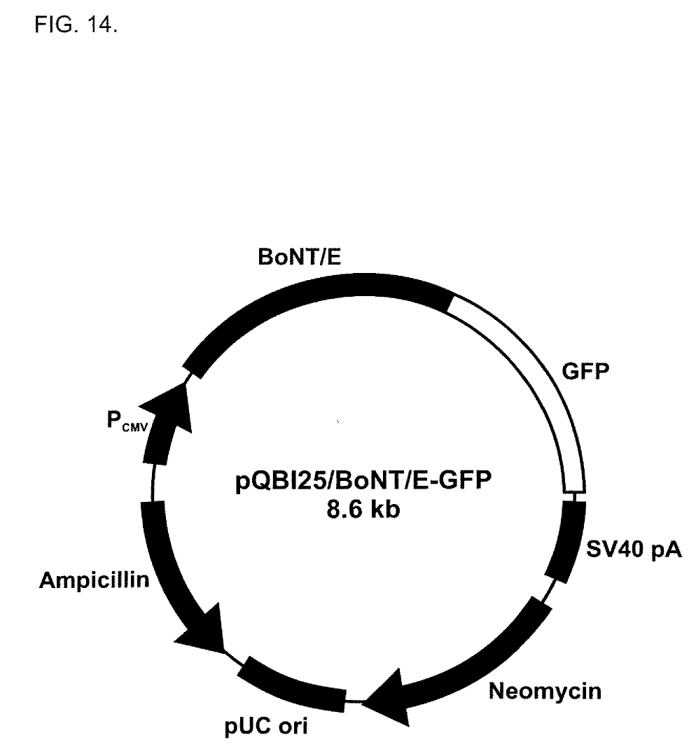

Restriction endonuclease sites suitable for cloning an operably linked nucleic acid molecule into a pQBI25 vector (Qbiogene, Inc., Carlsbad, Calif.) are incorporated into the 5′- and 3′ ends of modified open reading frame SEQ ID NO: 97. This nucleic acid molecule is synthesized and a pUCBHB1/BoNT/E construct is obtained as described in Example 3. This construct is digested with restriction enzymes that 1) excise the insert containing the open reading frame of SEQ ID NO: 97 encoding an active BoNT/E; and 2) enable this insert to be operably-linked to a pQBI25 vector. This insert is subcloned using a T4 DNA ligase procedure into a pQBI25 vector that is digested with appropriate restriction endonucleases to yield pQBI25/BoNT/E-GFP (FIG. 14). The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yields a mammalian expression construct encoding an active BoNT/E operably linked to carboxy-terminal GFP peptide. A similar cloning strategy is used to make a pQBI 25 construct containing the unmodified open reading frame of SEQ ID NO: 3 used as a control for expression levels, as well as, to produce pQBI25 expression constructs in which any one of the modified open reading frames of SEQ ID NO: 74 through SEQ ID NO: 96 is operably linked to a pQBI25 vector.

To transiently express an active BoNT/E-GFP in a cell line, about 1.5×10⁵ SH-SY5Y cells are plated in a 35 mm tissue culture dish containing 3 mL of complete Dulbecco's Modified Eagle Media (DMEM), supplemented with 10% fetal bovine serum (FBS), 1× penicillin/streptomycin solution (Invitrogen, Inc, Carlsbad, Calif.) and 1× MEM non-essential amino acids solution (MEM) (Invitrogen, Inc, Carlsbad, Calif.), and grown in a 37° C. incubator under 5% carbon dioxide until cells reach a density of about 5×10⁵ cells/ml (6-16 hours). A 500 μL transfection solution is prepared by adding 250 μL of OPTI-MEM Reduced Serum Medium containing 15 μL of LIPOFECTAMINE 2000, a cationic liposome based reagent, (Invitrogen, Carlsbad, Calif.) incubated at room temperature for 5 minutes to 250 μL of OPTI-MEM Reduced Serum Medium containing 5 μg of a pQBI25/BoNT/E-GFP. This transfection was incubated at room temperature for approximately 20 minutes. The complete, supplemented DMEM media was replaced with 2 mL of OPTI-MEM Reduced Serum Medium and the 500 μL transfection solution was added to the SH-SY5Y cells and the cells incubated in a 37° C. incubator under 5% carbon dioxide for approximately 6 to 18 hours. Transfection media is replaced with 3 mL of fresh complete, supplemented DMEM and incubate cells in a 37° C. incubator under 5% carbon dioxide for 48 hours. Cells are harvest by rinsing cells once with 3.0 mL of 100 mM phosphate-buffered saline, pH 7.4 and lysing cells with a buffer containing 50 mM N-(2-hydroxyethyl)piperazine-N′-(2-ethanesulfonic acid) (HEPES), pH 6.8 150 mM sodium chloride, 1.5 mM magnesium chloride, 10% (v/v) glycerol, 1 mM ethylene glycol bis(β-aminoethyl ether) N,N,N′,N′-tetraacetic acid (EGTA), 2% (v/v) TRITON-X® 100 (4-octylphenol polyethoxylate) and 1× Complete protease inhibitor cocktail (Roche Applied Science, Indianapolis, Ind.). Cell samples are added to 2×LDS Sample Buffer (Invitrogen, Inc, Carlsbad, Calif.) and expression is measured by Western blot analysis (as described in Example 12) using either anti-BoNT/E or anti-GFP antibodies in order to identify pQBI25 constructs expressing increased amounts of BoNT/E-GFP produced from SEQ ID NO: 97 relative to constructs expressing BoNT/E-GFP from the SEQ ID NO: 3 control.

### Example 22

### Construction and Expression of pcDNA™6/BoNT/E-V5-His

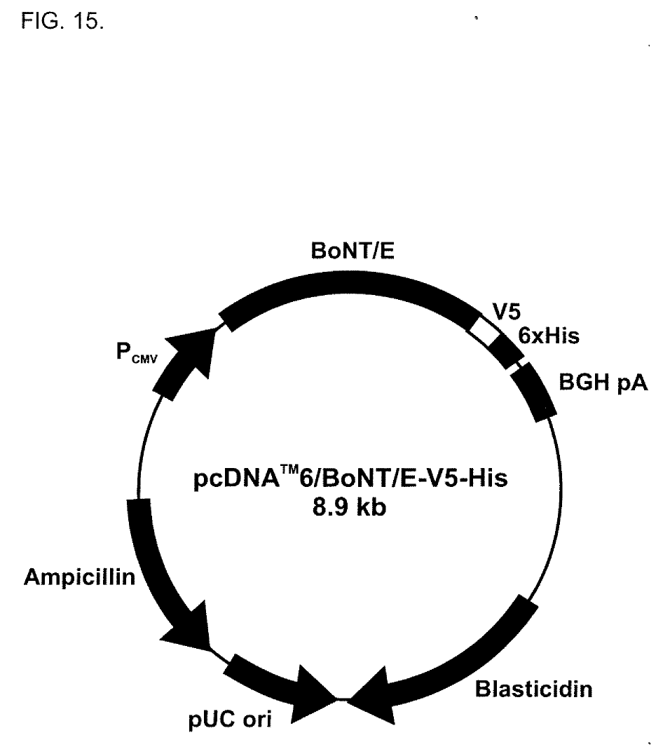

Restriction endonuclease sites suitable for cloning an operably linked nucleic acid molecule into a pcDNA™6 vector (Invitrogen, Inc, Carlsbad, Calif.) are incorporated into the 5′- and 3′ ends of modified open reading frame SEQ ID NO: 97. This nucleic acid molecule is synthesized and a pUCBHB1/BoNT/E construct obtained as described in Example 3. This construct is digested with restriction enzymes that 1) excise the insert containing the open reading frame of SEQ ID NO: 97 encoding an active BoNT/E; and 2) enable this insert to be operably-linked to a pcDNA™6 vector. This insert is subcloned using a T4 DNA ligase procedure into a pcDNA™6 vector that is digested with appropriate restriction endonucleases to yield pcDNA™6/BoNT/E-V5-His (FIG. 15). The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yields a mammalian expression construct encoding an active BoNT/E operably linked to carboxy-terminal V5 and polyhistidine binding peptides. A similar cloning strategy is used to make a pcDNA™6 construct containing the unmodified open reading frame of SEQ ID NO: 3 used as a control for expression levels, as well as, to produce pcDNA™6 expression constructs in which any one of the modified open reading frames of SEQ ID NO: 74 through SEQ ID NO: 96 is operably linked to a pcDNA™6 vector.

To transiently express BoNT/E-V5-His in a cell line, about 1.5×10⁵ SH-SY5Y cells are plated in a 35 mm tissue culture dish containing 3 mL of complete Dulbecco's Modified Eagle Media (DMEM), supplemented with 10% fetal bovine serum (FBS), 1× penicillin/streptomycin solution (Invitrogen, Inc, Carlsbad, Calif.) and 1× MEM non-essential amino acids solution (MEM) non-essential amino acids solution (Invitrogen, Inc, Carlsbad, Calif.), and grown in a 37° C. incubator under 5% carbon dioxide until cells reach a density of about 5×10⁵ cells/ml (6-16 hours). A 500 μL transfection solution is prepared by adding 250 μL of OPTI-MEM Reduced Serum Medium containing 15 μL of LIPOFECTAMINE 2000, a cationic liposome based reagent, (Invitrogen, Carlsbad, Calif.) incubated at room temperature for 5 minutes to 250 μL of OPTI-MEM Reduced Serum Medium containing 5 μg of a pcDNA™6/BoNT/E-V5-His. This transfection was incubated at room temperature for approximately 20 minutes. The complete, supplemented DMEM media was replaced with 2 mL of OPTI-MEM Reduced Serum Medium and the 500 μL transfection solution was added to the SH-SY5Y cells and the cells incubated in a 37° C. incubator under 5% carbon dioxide for approximately 6 to 18 hours. Transfection media is replaced with 3 mL of fresh complete, supplemented DMEM and cells are incubated in a 37° C. incubator under 5% carbon dioxide for 48 hours. Both media and cells are collected for expression analysis of the BoNT/E-V5-His peptide. Media is harvested by transferring the media to 15 mL snap-cap tubes and centrifuging tubes at 500×g for 5 minutes to remove debris. Cells are harvested by rinsing cells once with 3.0 mL of 100 mM phosphate-buffered saline, pH 7.4 and lysing cells with a buffer containing 62.6 mM 2-amino-2-hydroxymethyl-1,3-propanediol hydrochloric acid (Tris-HCl), pH 6.8 and 2% sodium lauryl sulfate (SDS). Both media and cell samples are added to 2×LDS Sample Buffer (Invitrogen, Inc, Carlsbad, Calif.) and expression is measured by Western blot analysis (as described in Example 12) using either anti-BoNT/E, anti-V5 or anti-His antibodies in order to identify pcDNA™6 constructs expressing increased amounts of BoNT/E-V5-His produced from SEQ ID NO: 97 relative to constructs expressing BoNT/E-V5-His from the SEQ ID NO: 3 control.

To generate a stably-integrated cell line expressing BoNT/E-V5-His, approximately 1.5×10⁵ SH-SY5Y cells are plated in a 35 mm tissue culture dish containing 3 mL of complete DMEM, supplemented with 10% FBS, 1× penicillin/streptomycin solution (Invitrogen, Inc, Carlsbad, Calif.) and 1×MEM non-essential amino acids solution (Invitrogen, Inc, Carlsbad, Calif.), and grown in a 37° C. incubator under 5% carbon dioxide until cells reach a density of about 5×10⁵ cells/ml (6-16 hours). A 500 μL transfection solution is prepared by adding 250 μL of OPTI-MEM Reduced Serum Medium containing 15 μL of LIPOFECTAMINE 2000, a cationic liposome based reagent, (Invitrogen, Carlsbad, Calif.) incubated at room temperature for 5 minutes to 250 μL of OPTI-MEM Reduced Serum Medium containing 5 μg of a pcDNA™6/BoNT/E-V5-His. This transfection was incubated at room temperature for approximately 20 minutes. The complete, supplemented DMEM media was replaced with 2 mL of OPTI-MEM Reduced Serum Medium and the 500 μL transfection solution was added to the SH-SY5Y cells and the cells incubated in a 37° C. incubator under 5% carbon dioxide for approximately 6 to 18 hours. Transfection media is replaced with 3 mL of fresh complete, supplemented DMEM and cells are incubated in a 37° C. incubator under 5% carbon dioxide for approximately 48 hours. Media is replaced with 3 mL of fresh complete DMEM, containing approximately 5 μg/mL of blasticidin, 10% FBS, 1× penicillin/streptomycin solution (Invitrogen, Inc, Carlsbad, Calif.) and 1× MEM non-essential amino acids solution (Invitrogen, Inc, Carlsbad, Calif.). Cells are incubated in a 37° C. incubator under 5% carbon dioxide for approximately 3-4 weeks, with old media being replaced with fresh blasticidin selective, complete, supplemented DMEM every 4 to 5 days. Once blasticidin-resistant colonies are established, resistant clones are replated to new 35 mm culture plates containing fresh complete DMEM, supplemented with approximately 5 μg/mL of blasticidin, 10% FBS, 1× penicillin/streptomycin solution (Invitrogen, Inc, Carlsbad, Calif.) and 1× MEM non-essential amino acids solution (Invitrogen, Inc, Carlsbad, Calif.), until these cells reach a density of 6 to 20×10⁵ cells/mL. To test for expression of BoNT/E-V5-His from SH-SY5Y cell lines that have stably-integrated a pcDNA™6/BoNT/E-V5-His, approximately 1.5×10⁵ SH-SY5Y cells from each cell line are plated in a 35 mm tissue culture dish containing 3 mL of blasticidin selective, complete, supplemented DMEM and grown in a 37° C. incubator under 5% carbon dioxide until cells reach a density of about 5×10⁵ cells/ml (6-16 hours). Media is replaced with 3 mL of fresh blasticidin selective, complete, supplemented DMEM and cells are incubated in a 37° C. incubator under 5% carbon dioxide for 48 hours. Both media and cells are collected for expression analysis of BoNT/E-V5-His. Media is harvested by transferring the media to 15 mL snap-cap tubes and centrifuging tubes at 500×g for 5 minutes to remove debris. Cells are harvest by rinsing cells once with 3.0 mL of 100 mM phosphate-buffered saline, pH 7.4 and lysing cells with a buffer containing 62.6 mM 2-amino-2-hydroxymethyl-1,3-propanediol hydrochloric acid (Tris-HCl), pH 6.8 and 2% sodium lauryl sulfate (SDS). Both media and cell samples are added to 2×LDS Sample Buffer (Invitrogen, Inc, Carlsbad, Calif.) and expression is measured by Western blot analysis (as described in Example 12) using either anti-BoNT/E, anti-V5 or anti-His antibodies in order to identify SH-SY5Y cell lines expressing increased amounts of BoNT/E-V5-His produced from SEQ ID NO: 97 relative to cell lines expressing BoNT/E-V5-His from the SEQ ID NO: 3 control. The established SH-SY5Y cell line showing the highest expression level of BoNT/E-V5-His relative to the SEQ ID NO: 3 control is selected for large-scale expression using 3 L flasks. Procedures for large-scale expression are as outlined above except the starting volume is approximately 800-1000 mL of complete DMEM and concentrations of all reagents are proportionally increased for this volume. For greater details on all procedures described in this example, see pcDNA™6/V5-His A, B, and C, version C, 28-0183 (Invitrogen, Inc, Carlsbad, Calif.).

### Example 23

### Construction and Expression of pIVEX2.3d/BoNT/E-His

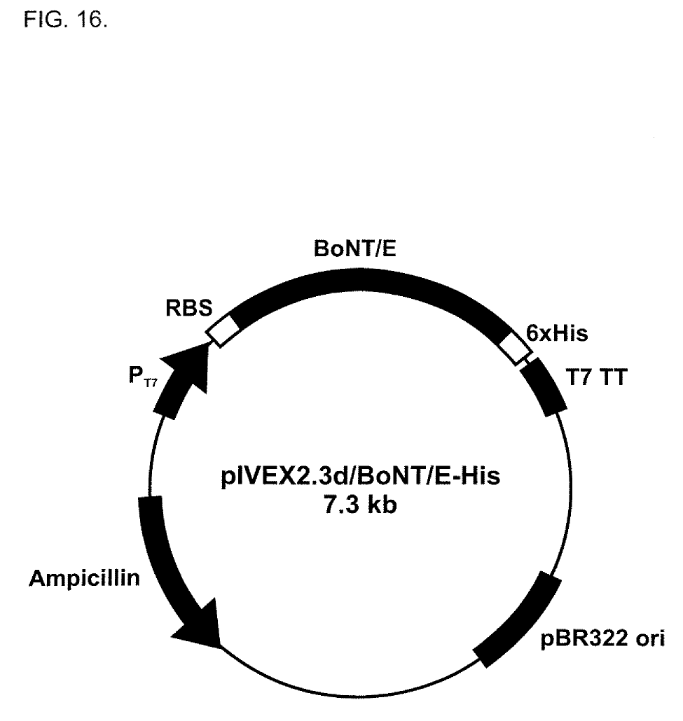

Restriction endonuclease sites suitable for cloning an operably linked nucleic acid molecule into a pIVEX2.3d vector (Roche Applied Science, Indianapolis, Ind.) are incorporated into the 5′- and 3′ ends of modified open reading frame SEQ ID NO: 4. This nucleic acid molecule is synthesized and a pUCBHB1/BoNT/E construct obtained as described in Example 3. This construct is digested with restriction enzymes that 1) excise the insert containing the open reading frame of SEQ ID NO: 4 encoding an active BoNT/E; and 2) enable this insert to be operably-linked to a pIVEX2.3d vector. This insert is subcloned using a T4 DNA ligase procedure into a pIVEX2.3d vector that is digested with appropriate restriction endonucleases to yield pIVEX2.3d/BoNT/E-His (FIG. 16). The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yields a prokaryotic expression construct encoding an active BoNT/E operably linked to a carboxy-terminal polyhistidine binding peptide. A similar cloning strategy is used to make a pIVEX2.3d construct containing the unmodified open reading frame of SEQ ID NO: 3 used as a control for expression levels, as well as, to produce pIVEX2.3d expression constructs in which any one of the modified open reading frames of SEQ ID NO: 5 through SEQ ID NO: 34 is operably linked to a pIVEX2.3d vector.

The RTS 100 E. coli HY Kit (Roche Applied Science, Indianapolis, Ind.) is used to express an active BoNT/E using a cell-free expression system. A 50 μl reaction mixture consisting of 12 μl E. coli lysate, 10 μl reaction mix, 12 μl amino acids, 1 μl methionine, 5 μl reconstitution buffer and 0.5 μg of pIVEX2.3d/BoNT/E-His is incubated in a 30° C. thermomixer for 4-6 hours. A 5 μl sample from this reaction mixture is added to 2×LDS Sample Buffer (Invitrogen, Inc, Carlsbad, Calif.) and expression is measured by Western blot analysis (as described in Example 12) using either anti-BoNT/E or anti-His antibodies in order to identify pIVEX2.3d constructs expressing increased amounts of BoNT/E-His produced from SEQ ID NO: 4 relative to constructs expressing BoNT/E-His from SEQ ID NO: 3. Procedures for large-scale expression are as outlined above except the RTS 9000 E. coli HY Kit (Roche Applied Science, Indianapolis, Ind.) is used. For greater details on all procedures described in this example, see RTS 100 E. coli HY Kit, In vitro protein synthesis system based on E. coli lysate, Instruction Manual, version 3, October 2003 (Roche Applied Science, Indianapolis, Ind.) and Rapid Translation System RTS 9000 E. coli HY Kit, In vitro protein synthesis system based on an enhanced E. coli lysate, Instruction Manual, version 3, November 2001 (Roche Applied Science, Indianapolis, Ind.).

Although aspects of the present invention have been described with reference to the disclosed embodiments, one skilled in the art will readily appreciate that the specific experiments disclosed are only illustrative of these aspects and in no way limit the present invention. Various modifications can be made without departing from the spirit of the present invention.

What is claimed:

1. A nucleic acid molecule comprising a modified open reading frame encoding an active BoNT/E;
wherein the active BoNT/E is SEQ ID NO: 1; and
wherein the modified open reading frame comprises a nucleic acid sequence having at least 95% nucleic acid sequence identity to SEQ ID NO: 4.

2. The molecule according to claim 1, wherein the molecule is an expression construct.

3. An Escherichia coli cell comprising an expression construct, wherein the expression construct comprises the open reading frame of claim 1.

4. The cell according to claim 3, wherein the expression construct is transiently contained in the Escherichia coli cell.

5. The cell according to claim 3, wherein the expression construct is stably contained in the Escherichia coli cell.


SEQUENCE LISTING (140 sequences)

SEQ ID NO: 1 - protein, 1252 aa, Clostridium botulinum
  DOMAIN (1)...(422): Light Chain; enzymatic domain; therapeutic domain; catalytic domain (active)
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 2 - protein, 1251 aa, Clostridium botulinum
  DOMAIN (1)...(422): Light Chain; enzymatic domain; therapeutic domain; catalytic domain (inactive)
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 3 - DNA, 3759 bp, Clostridium botulinum
  mat_peptide (1)...(3756): BoNT/E, unmodified
atgccaaaaattaatagttttaattataatgatcctgttaatgatagaacaattttatatattaaaccaggcggttgtcaagaattttataaatcatttaatattatgaaaaatatttggataattccagagagaaatgtaattggtacaaccccccaagattttcatccgcctacttcattaaaaaatggagatagtagttattatgaccctaattatttacaaagtgatgaagaaaaggatagatttttaaaaatagtcacaaaaatatttaatagaataaataataatctttcaggagggattttattagaagaactgtcaaaagctaatccatatttagggaatgataatactccagataatcaattccatattggtgatgcatcagcagttgagattaaattctcaaatggtagccaagacatactattacctaatgttattataatgggagcagagcctgatttatttgaaactaacagttccaatatttctctaagaaataattatatgccaagcaatcacggttttggatcaatagctatagtaacattctcacctgaatattcttttagatttaatgataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggaaatcaatatgtatcgaaataaataatggtgagttattttttgtggcttccgagaatagttataatgatgataatataaatactcctaaagaaattgacgatacagtaacttcaaataataattatgaaaatgatttagatcaggttattttaaattttaatagtgaatcagcacctggactttcagatgaaaaattaaatttaactatccaaaatgatgcttatataccaaaatatgattctaatggaacaagtgatatagaacaacatgatgttaatgaacttaatgtatttttctatttagatgcacagaaagtgcccgaaggtgaaaataatgtcaatctcacctcttcaattgatacagcattattagaacaacctaaaatatatacatttttttcatcagaatttattaataatgtcaataaacctgtgcaagcagcattatttgtaagctggatacaacaagtgttagtagattttactactgaagctaaccaaaaaagtactgttgataaaattgcagatatttctatagttgttccatatataggtcttgctttaaatataggaaatgaagcacaaaaaggaaattttaaagatgcacttgaattattaggagcaggtattttattagaatttgaacccgagcttttaattcctacaattttagtattcacgataaaatcttttttaggttcatctgataataaaaataaagttattaaagcaataaataatgcattgaaagaaagagatgaaaaatggaaagaagtatatagttttatagtatcgaattggatgactaaaattaatacacaatttaataaaagaaaagaacaaatgtatcaagctttacaaaatcaagtaaatgcaattaaaacaataatagaatctaagtataatagttatactttagaggaaaaaaatgagcttacaaataaatatgatattaagcaaatagaaaatgaacttaatcaaaaggtttctatagcaatgaataatatagacaggttcttaactgaaagttctatatcctatttaatgaaattaataaatgaagtaaaaattaataaattaagagaatatgatgagaatgtcaaaacgtatttattgaattatattatacaacatggatcaatcttgggagagagtcagcaagaactaaattctatggtaactgataccctaaataatagtattccttttaagctttcttcttatacagatgataaaattttaatttcatattttaataaattctttaagagaattaaaagtagttcagttttaaatatgagatataaaaatgataaatacgtagatacttcaggatatgattcaaatataaatattaatggagatgtatataaatatccaactaataaaaatcaatttggaatatataatgataaacttagtgaagttaatatatctcaaaatgattacattatatatgataataaatataaaaattttagtattagtttttgggtaagaattcctaactatgataataagatagtaaatgttaataatgaatacactataataaattgtatgagagataataattcaggatggaaagtatctcttaatcataatgaaataatttggacattgcaagataatgcaggaattaatcaaaaattagcatttaactatggtaacgcaaatggtatttctgattatataaataagtggatttttgtaactataactaatgatagattaggagattctaaactttatattaatggaaatttaatagatcaaaaatcaattttaaatttaggtaatattcatgttagtgacaatatattatttaaaatagttaattgtagttatacaagatatattggtattagatattttaatatttttgataaagaattagatgaaacagaaattcaaactttatatagcaatgaacctaatacaaatattttgaaggatttttggggaaattatttgctttatgacaaagaatactatttattaaatgtgttaaaaccaaataactttattgataggagaaaagattctactttaagcattaataatataagaagcactattcttttagctaatagattatatagtggaataaaagttaaaatacaaagagttaataatagtagtactaacgataatcttgttagaaagaatgatcaggtatatattaattttgtagccagcaaaactcacttatttccattatatgctgatacagctaccacaaataaagagaaaacaataaaaatatcatcatctggcaatagatttaatcaagtagtagttatgaattcagtaggaaataattgtacaatgaattttaaaaataataatggaaataatattgggttgttaggtttcaaggcagatactgtagttgctagtacttggtattatacacatatgagagatcatacaaacagcaatggatgtttttggaactttatttctgaagaacatggatggcaagaaaaataa

SEQ ID NO: 4 - DNA, 3759 bp, Artificial Sequence
  mat_peptide (1)...(3756): BoNT/E, E. coli-modified 1
atgcctaaaatcaattcgttcaactataatgacccggttaacgatcgcacgatcctgtatatcaagccaggtggatgtcaagaattttataaatcattcaacatcatgaaaaatatttggattatcccggaacgcaacgtgatcggcacgacgcctcaagattttcacccgccgacctccctgaaaaatggcgacagttcctactatgacccgaattatttacaatcggatgaagaaaaagatcgtttcctcaagatcgtcacgaaaattttcaaccgcatcaataacaatctgtccggtggcatcttacttgaggaattatctaaagctaatccgtatctggggaacgataataccccggataatcagttccacattggcgatgcgagcgctgtggaaattaaattcagcaacggcagtcaagatattcttctcccaaacgtgattatcatgggggctgaacctgatcttttcgaaactaatagttccaatatttcactgcgcaataattatatgccgtcgaaccatggctttggctcaatcgcaattgtgacgttctcacctgaatatagttttcgttttaacgacaacagcatgaatgaatttatccaagacccggcgctgactttgatgcatgaactgatccatagcttgcacggcctgtatggcgctaaaggcatcactaccaaatacacgattacgcaaaaacaaaatcccttaatcaccaacatccgcggcaccaacattgaagaatttctgaccttcggcggaacggatctgaacatcattacatctgcccaaagcaacgacatctataccaatctgttagcagattataagaaaatcgccagcaaattatctaaagttcaggtcagcaatccgctgttaaacccgtataaagatgtgttcgaagcgaaatacggcttggacaaagacgctagtggcatctattccgtcaatattaataaatttaacgatattttcaaaaaattatattccttcaccgaatttgatttggccaccaaattccaggtcaaatgtcgtcaaacctatattggccaatacaaatattttaaactgagcaacctgcttaatgattccatctacaatattagtgaaggttacaatattaataacctgaaagttaactttcgtgggcaaaatgcgaatctgaacccccgcatcattacacccatcacgggccgtgggttggtcaaaaaaattattcgcttttgtaagaatatcgtgagcgtgaagggtattcgcaaaagtatctgtatcgaaatcaataatggcgaactgtttttcgtcgcatctgaaaactcgtataacgatgacaatatcaacacaccgaaagaaattgatgacactgtcacttctaacaacaattacgaaaacgacctggaccaggtgatcctcaatttcaatagcgaaagcgcacccggcctgagcgatgaaaaacttaatctcacgattcagaacgacgcctacattccaaaatacgatagtaatggtacatctgatattgaacagcatgatgtcaacgaattaaatgttttcttttacctcgatgcccagaaagtgccggaaggtgagaacaacgtaaatctgacctcttcgattgatacggcattattagaacagccgaaaatttatactttcttttcgtccgaatttattaacaatgttaacaaaccggttcaagcggcgttattcgtttcctggattcagcaagttcttgtagattttacaaccgaggctaatcagaagagcacggtggataagatcgccgacatcagcatcgtcgtgccctacattggtttggcattaaacattggtaatgaggcgcaaaaggggaactttaaagacgccctggaattattaggagcaggtattctgctggagttcgaacctgagctgctgattccgactattttagtgttcaccattaaatccttcttaggctctagtgacaacaaaaataaagtgattaaagcgatcaataatgcccttaaagaacgtgatgagaaatggaaagaagtctactccttcattgtctcaaattggatgacgaaaatcaacacgcagtttaataaacgcaaagaacagatgtatcaggcgctgcaaaaccaggttaatgcgatcaagacaattattgaatctaagtacaactcgtacaccctggaggagaaaaatgaactgactaataagtacgatattaaacaaatcgaaaacgaattgaatcagaaagtctccatcgctatgaacaatatcgatcgctttctgaccgaaagctctatttcctatttgatgaaacttatcaatgaagtcaaaatcaacaaacttcgcgaatatgatgagaacgtaaaaacgtacctgctcaattatattattcaacatgggtcgattctgggcgagtctcaacaagaattgaactcgatggtgacggatactttgaataactcgattccgtttaaattatcgtcatacaccgatgataaaattcttatctcgtacttcaacaaattctttaagcggatcaaaagcagcagcgtccttaatatgcgctataaaaacgataagtacgtagatacgtctggatacgacagtaacattaatattaatggggacgtctataaatatccgacaaataaaaaccaattcgggatttataatgataaactttcggaggtgaacatcagccagaacgattatattatttacgataataaatacaaaaacttcagcatttctttttgggtgcgtatcccaaattacgacaacaaaattgtgaacgtgaataacgaatacacgatcattaattgcatgcgcgataacaattctggttggaaagttagcctgaatcacaatgagattatctggactcttcaggacaatgctggtatcaaccaaaaattagcgttcaactacggtaatgccaacggtatttctgactacatcaataagtggatctttgtgaccatcaccaatgaccgcctcggcgatagcaagctgtacattaacggtaacctgatcgaccagaaatctattctgaacctgggtaacattcacgtaagtgacaacatcctttttaaaattgtcaattgctcgtatactcgttatatcggcattcgctatttcaatattttcgacaaagaactggatgagacggaaatccagactctgtattctaacgaaccgaacaccaacatcctgaaggacttttgggggaattatcttctctacgataaagagtactaccttcttaacgtgttgaagccgaacaacttcattgatcgtcgtaaggatagcaccttgagcattaacaacattcgtagcaccattttactggcaaaccgcctgtacagcggcattaaagtcaaaattcagcgtgtcaataactccagtacgaatgacaatctggtgcggaaaaatgaccaagtctatattaactttgtcgcaagcaaaactcacctctttccattatatgcggatacagctaccaccaataaagaaaaaactattaaaatctcctcttccgggaaccgctttaatcaggtggtagttatgaactcggtcggcaacaattgtactatgaattttaaaaataataacggcaataacatcggcctgctgggcttcaaagctgatacagttgtggccagcacctggtattacacccacatgcgtgatcataccaatagtaatggctgcttttggaattttatttctgaagagcacggctggcaagaaaaataa

SEQ ID NO: 5 - DNA, 3759 bp, Artificial Sequence
  mat_peptide (1)...(3756): BoNT/E, E. coli-modified 2
atgcccaaaattaattcctttaactataatgacccggtaaacgatcggacgattctatacatcaaaccgggtggctgtcaggaattctataagtcttttaatatcatgaaaaacatctggattatcccggaacgaaatgtgattggcactacaccacaagattttcatcccccaactagcctgaaaaatggtgattctagctattacgatccgaattatttacagagtgatgaagagaaagataggttcctgaaaatcgtgacgaaaattttcaaccggattaataacaatctgagtggcggtattctgttagaagagttaagtaaagccaatccgtacttaggtaatgataataccccagataatcagttccatatcggcgatgcttcggcggtcgagattaaatttagtaacggcagccaggatattttactccccaacgtaattatcatgggggcagagcctgatctctttgaaaccaatagttctaacataagcctgcgcaacaattatatgccctccaaccacggcttcggttcaattgcgattgttacgttttcgcctgagtactcttttaggttcaacgacaatagcatgaacgaatttattcaggatccggccctgaccttgatgcacgaactaatccacagtctccatggactgtacggagcgaaaggaattaccacaaagtacaccataacccagaaacagaatccgctcataaccaatattcgtggcaccaacattgaagagtttcttacgtttggtggcacagatcttaatattatcacctctgctcagagcaacgatatatatacgaacttattggcggactacaagaaaatcgcatcgaaactttcaaaagttcaggtctccaacccgctgctcaatccgtataaggatgtcttcgaagcgaaatatggccttgacaaagatgcgtcgggcatatacagcgtgaatattaacaaattcaacgatatctttaaaaagctgtactctttcaccgagtttgatctggccacaaaatttcaagtgaaatgtcgccagacgtacattggtcagtacaaatattttaaactgtcaaaccttctgaatgactccatctataatatcagtgaagggtataatatcaataacctgaaggtaaattttcgtggccaaaacgcgaacttgaacccgcgcatcattactccgatcacggggagaggcctggtaaaaaagattatccgcttctgcaaaaacattgtaagcgtgaaaggtatccggaaaagcatttgcattgaaatcaataacggggagttatttttcgtggcctcagaaaatagctataacgatgacaatattaacaccccaaaagaaatcgatgacacagtcacgagcaataacaattatgaaaatgatctggatcaggtcatcctgaatttcaattctgaaagcgccccaggtttatcagatgaaaaactgaatctgaccatacaaaatgatgcgtatattccgaaatacgactcaaatggaacctcggacatcgaacagcatgacgtgaatgaattaaacgtttttttctacctggacgcacagaaagttccagaaggtgaaaataacgtgaatctgactagctctattgatactgccttattggaacagcctaaaatttatacatttttctcttcagaatttatcaacaatgttaacaaaccggtccaggcggctctgtttgtcagttggattcagcaagtactggttgattttaccacagaagctaatcaaaagtctactgttgacaaaatcgcggacatctcgatagttgtaccatacattggcctcgccctgaatataggcaatgaagcacaaaaggggaatttcaaagatgcactcgaattgctgggggcggggattctgttggagtttgaaccggagcttctgattccgacaatccttgtttttacgattaagagttttctgggaagctccgacaataaaaataaagtgattaaagcgatcaacaatgctctgaaagaacgcgacgaaaaatggaaggaagtctattccttcattgtttcgaactggatgactaaaattaacacacaattcaacaagcgtaaagaacaaatgtatcaagcattgcagaatcaagttaatgccatcaaaaccattatcgagtctaaatataacagttataccctggaagagaaaaacgagctgactaataaatatgacatcaaacagattgagaatgaattgaaccaaaaggtgtcgattgcaatgaacaatattgaccgtttcctgactgagagtagcatctcttatctgatgaaactgattaacgaggttaaaatcaacaagctgcgcgaatatgatgagaatgtcaagacttatctgctaaactacatcattcagcacggaagtatacttggagagagccagcaagaactcaacagtatggttaccgataccctcaacaattcaattcctttcaaattaagctcgtacactgatgacaagattctgataagctattttaataagttctttaaacgaatcaagtcctcttcagttcttaatatgcgttacaaaaacgacaaatacgtggatacgtcaggttacgacagcaatatcaacattaacggggatgtttataaataccccacgaataagaaccagtttggtatatataatgataagctgtccgaagtaaatatctctcagaacgattatattatatacgacaacaaatacaaaaatttctcgatcagtttctgggtcagaattccgaattatgataacaaaatcgtgaacgtgaacaatgaatatacgattataaattgcatgcgtgacaacaattcaggctggaaagtgtctttgaatcataatgaaattatatggacgttacaggataacgctggcattaaccagaaattggcctttaattatggcaacgctaacggtatctcagactatataaataaatggattttcgtcaccatcaccaatgatcgcctgggtgattcgaaactgtatattaacggcaacttgatcgaccagaaatcgattcttaacttaggcaacattcatgtctccgataatatcctatttaagatcgttaattgctcctatacccgttatattggtatacgctacttcaacatttttgacaaggaactagatgagaccgaaattcagactctgtatagcaatgagcctaatacaaatatcctgaaagatttttggggtaactacctgttgtacgataaagaatactatctgttaaacgtgttgaaacctaataactttattgatcgtcgcaaagattcgactctgtccatcaacaatattcgcagtacgatcctgcttgccaatcgtctttacagcggtattaaggtgaagatccagcgtgttaacaatagttccacaaacgacaacctcgttcgtaagaacgatcaggtctatattaacttcgtggcatccaaaacacacctgttcccgctgtatgcggacacagccacgaccaacaaagaaaagaccatcaaaatcagctcttcaggtaaccggtttaaccaagtggtagtgatgaactcagtgggtaataactgtaccatgaattttaaaaacaataacggtaacaatattgggctgttaggctttaaagctgataccgtagtggcatccacctggtattatacgcacatgcgcgatcatacgaacagcaacggatgtttttggaattttatttctgaagagcatggctggcaagaaaaataa

SEQ ID NO: 6 - DNA, 3759 bp, Artificial Sequence
  mat_peptide (1)...(3756): BoNT/E, E. coli-modified 3
atgccgaaaattaatagctttaattataatgatccggtgaatgatcgtaccattctgtatattaaaccgggcggctgtcaggaattttataaaagctttaatattatgaaaaatatttggattattccggaacgtaatgtgattggcaccaccccgcaggattttcatccgccgaccagcctgaaaaatggcgatagcagctattatgatccgaattatctgcagagcgatgaagaaaaagatcgttttctgaaaattgtgaccaaaatttttaatcgtattaataataatctgagcggcggcattctgctggaagaactgagcaaagcgaatccgtatctgggcaatgataataccccggataatcagtttcatattggcgatgcgagcgcggtggaaattaaatttagcaatggcagccaggatattctgctgccgaatgtgattattatgggcgcggaaccggatctgtttgaaaccaatagcagcaatattagcctgcgtaataattatatgccgagcaatcatggctttggcagcattgcgattgtgacctttagcccggaatatagctttcgttttaatgataatagcatgaatgaatttattcaggatccggcgctgaccctgatgcatgaactgattcatagcctgcatggcctgtatggcgcgaaaggcattaccaccaaatataccattacccagaaacagaatccgctgattaccaatattcgtggcaccaatattgaagaatttctgacctttggcggcaccgatctgaatattattaccagcgcgcagagcaatgatatttataccaatctgctggcggattataaaaaaattgcgagcaaactgagcaaagtgcaggtgagcaatccgctgctgaatccgtataaagatgtgtttgaagcgaaatatggcctggataaagatgcgagcggcatttatagcgtgaatattaataaatttaatgatatttttaaaaaactgtatagctttaccgaatttgatctggcgaccaaatttcaggtgaaatgtcgtcagacctatattggccagtataaatattttaaactgagcaatctgctgaatgatagcatttataatattagcgaaggctataatattaataatctgaaagtgaattttcgtggccagaatgcgaatctgaatccgcgtattattaccccgattaccggccgtggcctggtgaaaaaaattattcgtttttgtaaaaatattgtgagcgtgaaaggcattcgtaaaagcatttgtattgaaattaataatggcgaactgttttttgtggcgagcgaaaatagctataatgatgataatattaataccccgaaagaaattgatgataccgtgaccagcaataataattatgaaaatgatctggatcaggtgattctgaattttaatagcgaaagcgcgccgggcctgagcgatgaaaaactgaatctgaccattcagaatgatgcgtatattccgaaatatgatagcaatggcaccagcgatattgaacagcatgatgtgaatgaactgaatgtgtttttttatctggatgcgcagaaagtgccggaaggcgaaaataatgtgaatctgaccagcagcattgataccgcgctgctggaacagccgaaaatttataccttttttagcagcgaatttattaataatgtgaataaaccggtgcaggcggcgctgtttgtgagctggattcagcaggtgctggtggattttaccaccgaagcgaatcagaaaagcaccgtggataaaattgcggatattagcattgtggtgccgtatattggcctggcgctgaatattggcaatgaagcgcagaaaggcaattttaaagatgcgctggaactgctgggcgcgggcattctgctggaatttgaaccggaactgctgattccgaccattctggtgtttaccattaaaagctttctgggcagcagcgataataaaaataaagtgattaaagcgattaataatgcgctgaaagaacgtgatgaaaaatggaaagaagtgtatagctttattgtgagcaattggatgaccaaaattaatacccagtttaataaacgtaaagaacagatgtatcaggcgctgcagaatcaggtgaatgcgattaaaaccattattgaaagcaaatataatagctataccctggaagaaaaaaatgaactgaccaataaatatgatattaaacagattgaaaatgaactgaatcagaaagtgagcattgcgatgaataatattgatcgttttctgaccgaaagcagcattagctatctgatgaaactgattaatgaagtgaaaattaataaactgcgtgaatatgatgaaaatgtgaaaacctatctgctgaattatattattcagcatggcagcattctgggcgaaagccagcaggaactgaatagcatggtgaccgataccctgaataatagcattccgtttaaactgagcagctataccgatgataaaattctgattagctattttaataaattttttaaacgtattaaaagcagcagcgtgctgaatatgcgttataaaaatgataaatatgtggataccagcggctatgatagcaatattaatattaatggcgatgtgtataaatatccgaccaataaaaatcagtttggcatttataatgataaactgagcgaagtgaatattagccagaatgattatattatttatgataataaatataaaaattttagcattagcttttgggtgcgtattccgaattatgataataaaattgtgaatgtgaataatgaatataccattattaattgtatgcgtgataataatagcggctggaaagtgagcctgaatcataatgaaattatttggaccctgcaggataatgcgggcattaatcagaaactggcgtttaattatggcaatgcgaatggcattagcgattatattaataaatggatttttgtgaccattaccaatgatcgtctgggcgatagcaaactgtatattaatggcaatctgattgatcagaaaagcattctgaatctgggcaatattcatgtgagcgataatattctgtttaaaattgtgaattgtagctatacccgttatattggcattcgttattttaatatttttgataaagaactggatgaaaccgaaattcagaccctgtatagcaatgaaccgaataccaatattctgaaagatttttggggcaattatctgctgtatgataaagaatattatctgctgaatgtgctgaaaccgaataattttattgatcgtcgtaaagatagcaccctgagcattaataatattcgtagcaccattctgctggcgaatcgtctgtatagcggcattaaagtgaaaattcagcgtgtgaataatagcagcaccaatgataatctggtgcgtaaaaatgatcaggtgtatattaattttgtggcgagcaaaacccatctgtttccgctgtatgcggataccgcgaccaccaataaagaaaaaaccattaaaattagcagcagcggcaatcgttttaatcaggtggtggtgatgaatagcgtgggcaataattgtaccatgaattttaaaaataataatggcaataatattggcctgctgggctttaaagcggataccgtggtggcgagcacctggtattatacccatatgcgtgatcataccaatagcaatggctgtttttggaattttattagcgaagaacatggctggcaggaaaaataa

SEQ ID NO: 7 - DNA, 3759 bp, Artificial Sequence
  mat_peptide (1)...(3756): BoNT/E, E. coli-modified 4
atgccaaaaattaattcgttcaattataatgatccagttaacgatcgcacaatcctctacatcaaacctggtggctgccaggaattctacaaatcattcaacattatgaagaatatttggatcattcccgaacgtaacgtcattgggaccactccgcaggacttccatccgccaacgtctctgaaaaacggggacagctcttattacgatcctaactatctgcagtcggatgaagagaaagatcgtttcctgaaaattgtaactaaaattttcaaccgcattaacaataacttgtcaggtgggattctgttagaagagctgagcaaagcgaacccctatctcggtaacgataatacaccggacaaccagtttcatattggagacgcgtcggcagtggaaattaaattcagcaatggttctcaggatattttactgcctaatgtgattatcatgggggcggagccggatctgtttgaaaccaatagctccaatattagtttacgtaacaattacatgccgagcaaccacggtttcggaagcattgccatcgttacctttagtcccgaatatagttttcgttttaatgataatagtatgaatgaatttattcaagaccctgccctgacacttatgcacgagcttatccactcactgcacggcctgtatggagccaagggcattaccactaagtatactattacgcaaaaacagaatccgctgattacaaatatccgtggcactaatattgaagagttcttgaccttcggcggtaccgatctcaatattatcacatctgcacagtcaaatgacatctacacaaacctgctcgcagactataaaaagattgcgtcgaaactgtctaaagtccaagtctctaacccactgttgaacccctacaaagatgtgttcgaagcaaagtacgggttggataaggatgcatcaggtatttatagtgtcaatattaacaagtttaatgacatcttcaaaaagttgtatagctttacggaatttgacctggcgaccaaatttcaggtgaaatgccggcaaacctacattggtcagtacaaatattttaaactgagcaacttgctgaatgattcgatttataatatttccgaagggtacaatatcaacaatcttaaagtgaactttcgcggtcagaatgcgaatctgaatccgcgtatcattacaccgattactggccgcggcctcgtgaaaaagatcattcgcttctgcaaaaatatcgtgagcgttaaaggcatccgtaaatccatttgtatcgagattaataacggcgaattgtttttcgttgcttccgagaacagttacaacgacgataatatcaacacgccgaaagagatcgacgatactgttaccagtaacaataactacgaaaatgatcttgatcaggtaattcttaatttcaactcggaatctgccccaggacttagcgacgaaaaactgaacctgacgatccagaacgatgcctatatcccgaaatatgattcaaacggtacatcagatatcgagcagcatgacgttaacgaattgaatgtgtttttctatctggacgctcagaaagtgcctgaaggcgagaacaatgtgaatctgacatcctctattgatacggcgttacttgaacaaccgaaaatctatacctttttcagttctgaatttattaacaatgttaataaaccggtgcaggcagcgctgttcgtctcatggattcagcaagtgcttgtagattttactaccgaggctaatcaaaaatctacggtggacaaaatcgcggacatcagcattgtggtcccttacatcggtctggccctgaacattgggaatgaagcacagaaaggtaacttcaaggatgccttggaactcctgggcgcagggatcttacttgaatttgaaccggaactgcttattccgacgatcctggtgtttaccattaagagttttctgggcagttcagacaataaaaacaaagtgatcaaagcgattaataacgcgcttaaagaacgtgatgaaaaatggaaagaagtatattcgtttattgtatcgaattggatgaccaaaatcaatacgcagtttaacaaacgtaaagagcagatgtaccaggcgctgcaaaaccaggtcaacgctattaagaccatcattgagagtaaatataatagctatacgctcgaagagaaaaacgaattaacgaacaagtatgatattaagcaaatcgaaaacgagttaaatcaaaaagtttctatcgctatgaacaatatcgaccgtttcctgaccgaatcaagcattagctacttaatgaagctgattaatgaagtgaagattaataaactgcgggaatacgatgagaatgtaaaaacatatttactgaactacattatccagcacggaagcatcctgggcgaatctcaacaggagctgaacagtatggtgaccgatactttaaacaattctatcccctttaaactgagcagttacacggatgacaaaatcctgatttcatatttcaataaatttttcaaacgcattaaatcttccagtgtattgaacatgcgctataaaaatgacaagtatgtcgatacttctggttatgatagcaacatcaacattaacggcgatgtttacaaatacccaaccaataaaaaccaatttggcatttataacgataagctgtccgaggttaacatctcacagaacgattatattatctatgacaacaaatacaagaacttttcaatttccttttgggtccgcatcccgaactacgacaataaaatcgtcaacgttaataacgaatatacaattatcaattgtatgcgtgataataactccggttggaaggtcagcctgaatcataacgaaattatctggacgttacaggataacgctggaatcaaccagaagctggcctttaattatggtaatgcgaacggaattagcgattatattaacaagtggatcttcgtgacaattactaatgatcgtctgggtgactccaagctgtacatcaatggaaatttaattgatcagaaatccattttaaacctgggtaacattcatgtatccgacaatattttgtttaaaattgtaaactgttcctatacccggtatatcggcattcgttacttcaacatttttgataaagaattagatgagacggaaattcaaaccctctattcgaacgagccgaatactaatattctgaaagatttttggggcaactatttgctttatgacaaagaatactatttactgaacgtcctgaaaccaaataacttcattgatcgtcgcaaggactccaccctgagtattaacaatatccgttcgaccattctgttggccaatcgcctgtactcgggtattaaagttaaaattcaacgggttaacaatagcagtacaaatgataacctcgttcgcaaaaatgatcaagtttatattaatttcgtcgccagcaaaacccatctgtttccgctgtatgctgataccgccactacgaataaggaaaaaacgattaagatttcgtcttcgggcaatcgttttaaccaggtggttgtgatgaattcagttggtaacaattgtaccatgaactttaaaaataacaatggcaataacattggcctgctcggttttaaagcagacaccgtagttgctagcacgtggtattacacccacatgcgtgatcataccaactctaatgggtgcttttggaattttattagcgaagagcatggctggcaggaaaaataa

SEQ ID NO: 8 - DNA, 3759 bp, Artificial Sequence
  mat_peptide (1)...(3756): BoNT/E, B. fragilis-modified 1
atgcccaagataaattcgtttaattataatgaccctgtgaatgatcgaactatcctgtatatcaaacctggcggatgtcaagaattttacaaatctttcaatataatgaaaaacatttggataatccccgagcggaatgtgataggaacaacgccccaggattttcatccgccaacgtctttgaagaatggtgactcgagttattacgatccgaactatctgcagtccgacgaagagaaagatcgttttcttaagattgtaacaaaaatttttaacagaataaataacaatttgtcaggaggcatccttctggaagagctgtcgaaagccaacccttatttgggaaatgataatactccagataatcagtttcatataggcgatgccagcgcggttgagatcaaattttccaatggttctcaagatattttgttacccaacgtgataattatgggcgccgaacctgacctgtttgaaaccaattcctctaatatatctttacgtaacaattatatgcccagtaaccatggattcggctctatcgctattgttacgttcagcccggagtattcatttagattcaacgataattcgatgaatgagttcattcaagatccagcacttacacttatgcacgaacttatccactcgctgcatggactttatggtgcgaaaggcataacgactaagtatacaatcactcaaaaacagaatccgctgattactaacattcgtggtacaaatatcgaagagtttttaaccttcggaggtacggatctaaatataattacaagcgcgcagagtaacgatatctatacaaatttgctggctgattataaaaagatagcttctaaattgagcaaagtccaggtgagcaaccctttgctgaatccgtacaaagatgttttcgaagcaaaatatggactcgataaggatgcctcaggaatctattctgtcaatataaataagtttaacgacatcttcaaaaagctgtattccttcactgagttcgatctcgccaccaagttccaggtaaagtgccgccaaacatatatcggtcagtataagtacttcaagttatcgaacctcttgaacgactcaatttataatattagcgaaggatacaatataaacaatcttaaagtaaacttccgcggccaaaacgccaatttaaacccgcgtattataacccctattaccgggcgggggttagtaaagaaaataatccgtttctgtaagaatatcgtctcggttaaaggcattagaaaatcgatctgtatcgaaatcaacaatggtgaattgttctttgtagcatcagaaaactcatataatgatgacaatatcaacacccctaaagagattgacgatactgttacatctaataacaattacgaaaacgatcttgaccaagttattcttaacttcaattctgaaagcgccccgggtttgagtgatgagaaattgaatctgactattcagaatgatgcctatatcccaaaatatgactccaacggtacctctgacatcgaacaacatgacgttaacgaactaaacgtctttttctaccttgacgctcagaaagtaccggaaggagaaaacaatgtgaacttaacgtcttcaatagacactgcattgctggagcagcctaagatttatacatttttctcgagtgaattcataaataacgtgaataaaccggttcaagcagccttattcgtgagttggatacagcaagtgctggttgatttcacaaccgaagccaatcaaaaatcgacagtagacaaaattgctgatatcagtatagtagttccctacatcggcctagctttgaatataggtaatgaagcccagaaaggcaatttcaaagatgcactggaacttctcggcgcaggtatcctgttggagttcgaacctgaattgcttatcccgaccattctcgtatttacaattaaaagctttctgggatcatcggacaataaaaataaagtgattaaggccattaataacgcattaaaggagcgcgacgaaaagtggaaagaagtctacagttttattgtctcaaattggatgaccaaaattaacacgcagtttaacaaacgcaaagagcagatgtaccaagctttacagaatcaggtgaatgcaataaagaccatcattgaaagtaagtacaacagttatacgttggaagagaaaaatgaattgactaataaatacgatatcaaacagattgaaaacgagctgaaccaaaaagtatctattgcaatgaataacatcgataggtttctaactgaaagtagcatcagctatctcatgaaactgatcaatgaagtaaaaatcaataagctgcgtgagtatgatgaaaatgtgaagacgtacttgttaaattacattatacaacatggttcgattctgggagaaagccaacaggaattgaatagtatggtaactgacacgctgaacaattccatcccgtttaaactctcgagctacacagacgataagatcctcatttcatattttaacaagttctttaaaaggattaaaagttcgagtgtactaaacatgcggtataaaaatgataagtatgtggacacatccggttacgattccaacattaacatcaatggagacgtgtataagtatccgactaataagaatcaatttgggatctataatgataaattatccgaggtcaacattagtcagaatgactacattatctatgataataaatacaagaacttttctataagcttttgggttcgcatccctaattacgacaacaaaattgtcaatgtaaataacgaatacaccataatcaattgcatgcgagataacaattccgggtggaaagtatctttaaaccataatgaaattatctggaccctgcaagataacgctggaataaatcaaaagcttgctttcaattatggaaatgctaacggaatctcagactatataaacaaatggatctttgtgacaataacgaacgatcggttgggggactctaagctgtatattaacggaaatctgattgatcagaagagtatcttgaacctgggtaatattcacgtatctgataacatattgttcaaaatagtaaattgttcgtatacgcgttatataggaatccgatattttaatatctttgacaaggaactggacgagactgaaatacaaacattatattcaaacgagcccaatacaaacattttaaaagatttctggggaaattatctcttgtatgacaaggaatattacctgctcaatgtgctgaaaccgaataactttatcgacagacggaaagatagtaccctttcgatcaataacattcgttctaccattttgcttgctaatcgcctgtattccggtattaaagtaaaaattcagcgtgtgaataactcatctactaacgataatctggtgcgtaagaatgatcaagtctatatcaactttgtcgcgagcaaaactcacctatttcccctttatgcagatactgcgaccacgaataaagagaaaaccataaaaatttccagttcaggtaacagattcaatcaagttgtagtgatgaactctgttggtaataactgcacaatgaatttcaaaaataacaatggtaataacattggattgttgggatttaaagccgataccgtagtagcttccacctggtattatacccacatgcgtgatcatactaattccaatgggtgtttttggaattttattagcgaagaacatgggtggcaggaaaagtaa

SEQ ID NO: 9 - DNA, 3759 bp, Artificial Sequence
  mat_peptide (1)...(3756): BoNT/E, B. fragilis-modified 2
atgccgaaaattaattcttttaattataatgatccggtaaatgatcgtaccattctgtatattaaaccgggaggatgtcaggaattttataaatcttttaatattatgaaaaatatttggattattccggaacgtaatgtaattggaaccaccccgcaggattttcatccgccgacctctctgaaaaatggagattcttcttattatgatccgaattatctgcagtctgatgaagaaaaagatcgttttctgaaaattgtaaccaaaatttttaatcgtattaataataatctgtctggaggaattctgctggaagaactgtctaaagccaatccgtatctgggaaatgataataccccggataatcagtttcatattggagatgcctctgccgtagaaattaaattttctaatggatctcaggatattctgctgccgaatgtaattattatgggagccgaaccggatctgtttgaaaccaattcttctaatatttctctgcgtaataattatatgccgtctaatcatggatttggatctattgccattgtaaccttttctccggaatattcttttcgttttaatgataattctatgaatgaatttattcaggatccggccctgaccctgatgcatgaactgattcattctctgcatggactgtatggagccaaaggaattaccaccaaatataccattacccagaaacagaatccgctgattaccaatattcgtggaaccaatattgaagaatttctgacctttggaggaaccgatctgaatattattacctctgcccagtctaatgatatttataccaatctgctggccgattataaaaaaattgcctctaaactgtctaaagtacaggtatctaatccgctgctgaatccgtataaagatgtatttgaagccaaatatggactggataaagatgcctctggaatttattctgtaaatattaataaatttaatgatatttttaaaaaactgtattcttttaccgaatttgatctggccaccaaatttcaggtaaaatgtcgtcagacctatattggacagtataaatattttaaactgtctaatctgctgaatgattctatttataatatttctgaaggatataatattaataatctgaaagtaaattttcgtggacagaatgccaatctgaatccgcgtattattaccccgattaccggacgtggactggtaaaaaaaattattcgtttttgtaaaaatattgtatctgtaaaaggaattcgtaaatctatttgtattgaaattaataatggagaactgttttttgtagcctctgaaaattcttataatgatgataatattaataccccgaaagaaattgatgataccgtaacctctaataataattatgaaaatgatctggatcaggtaattctgaattttaattctgaatctgccccgggactgtctgatgaaaaactgaatctgaccattcagaatgatgcctatattccgaaatatgattctaatggaacctctgatattgaacagcatgatgtaaatgaactgaatgtatttttttatctggatgcccagaaagtaccggaaggagaaaataatgtaaatctgacctcttctattgataccgccctgctggaacagccgaaaatttatacctttttttcttctgaatttattaataatgtaaataaaccggtacaggccgccctgtttgtatcttggattcagcaggtactggtagattttaccaccgaagccaatcagaaatctaccgtagataaaattgccgatatttctattgtagtaccgtatattggactggccctgaatattggaaatgaagcccagaaaggaaattttaaagatgccctggaactgctgggagccggaattctgctggaatttgaaccggaactgctgattccgaccattctggtatttaccattaaatcttttctgggatcttctgataataaaaataaagtaattaaagccattaataatgccctgaaagaacgtgatgaaaaatggaaagaagtatattcttttattgtatctaattggatgaccaaaattaatacccagtttaataaacgtaaagaacagatgtatcaggccctgcagaatcaggtaaatgccattaaaaccattattgaatctaaatataattcttataccctggaagaaaaaaatgaactgaccaataaatatgatattaaacagattgaaaatgaactgaatcagaaagtatctattgccatgaataatattgatcgttttctgaccgaatcttctatttcttatctgatgaaactgattaatgaagtaaaaattaataaactgcgtgaatatgatgaaaatgtaaaaacctatctgctgaattatattattcagcatggatctattctgggagaatctcagcaggaactgaattctatggtaaccgataccctgaataattctattccgtttaaactgtcttcttataccgatgataaaattctgatttcttattttaataaattttttaaacgtattaaatcttcttctgtactgaatatgcgttataaaaatgataaatatgtagatacctctggatatgattctaatattaatattaatggagatgtatataaatatccgaccaataaaaatcagtttggaatttataatgataaactgtctgaagtaaatatttctcagaatgattatattatttatgataataaatataaaaatttttctatttctttttgggtacgtattccgaattatgataataaaattgtaaatgtaaataatgaatataccattattaattgtatgcgtgataataattctggatggaaagtatctctgaatcataatgaaattatttggaccctgcaggataatgccggaattaatcagaaactggcctttaattatggaaatgccaatggaatttctgattatattaataaatggatttttgtaaccattaccaatgatcgtctgggagattctaaactgtatattaatggaaatctgattgatcagaaatctattctgaatctgggaaatattcatgtatctgataatattctgtttaaaattgtaaattgttcttatacccgttatattggaattcgttattttaatatttttgataaagaactggatgaaaccgaaattcagaccctgtattctaatgaaccgaataccaatattctgaaagatttttggggaaattatctgctgtatgataaagaatattatctgctgaatgtactgaaaccgaataattttattgatcgtcgtaaagattctaccctgtctattaataatattcgttctaccattctgctggccaatcgtctgtattctggaattaaagtaaaaattcagcgtgtaaataattcttctaccaatgataatctggtacgtaaaaatgatcaggtatatattaattttgtagcctctaaaacccatctgtttccgctgtatgccgataccgccaccaccaataaagaaaaaaccattaaaatttcttcttctggaaatcgttttaatcaggtagtagtaatgaattctgtaggaaataattgtaccatgaattttaaaaataataatggaaataatattggactgctgggatttaaagccgataccgtagtagcctctacctggtattatacccatatgcgtgatcataccaattctaatggatgtttttggaattttatttctgaagaacatggatggcaggaaaaataa

SEQ ID NO: 10 - DNA, 3759 bp, Artificial Sequence
  mat_peptide (1)...(3756): BoNT/E, B. fragilis-modified 3
atgcctaagattaacagtttcaattacaatgacccggtgaacgatagaaccattctgtacataaagccgggaggctgtcaagaattttataagtcatttaatattatgaaaaacatttggatcataccggaaagaaatgtaattggaacaactccgcaagattttcacccccctacgtccctgaaaaatggagacagttcttattacgatcctaactatcttcagtcggacgaagagaaagaccgttttttgaaaatagtaacgaaaatcttcaaccgcatcaataacaatttgtccggtggaatcctccttgaagagttgtcaaaagcaaacccgtatcttggaaacgataatactcccgataaccagttccatataggagacgcctcggccgtagagataaagttttctaacggaagtcaggatatcttattgcccaatgtaatcataatgggcgcagagcctgatctgtttgaaactaacagttccaacatttctctgcgcaataactatatgccgtccaaccatggtttcggcagcattgcaatcgttactttctcccctgaatatagttttcgttttaacgataacagtatgaatgagttcatccaagaccctgccctgacacttatgcatgagcttatacactcgcttcacggattatatggcgcaaagggaattaccacaaagtataccataacccaaaagcagaatcccctgattactaacatacgtggtactaacatagaagagtttttgacgttcggaggtacagacttgaatataatcacgtcagcccagtccaacgatatctacacgaatctgttggcagattacaaaaagatcgctagtaaactgtccaaggtacaggtctctaacccgttactgaatccttacaaagatgtttttgaggctaaatatggacttgacaaagatgcttctggtatctattctgttaatatcaacaaatttaacgatatttttaagaaactttatagttttacggagtttgaccttgccacaaaattccaagttaaatgtcgtcagacttatattggtcaatacaaatatttcaaattatcaaacttactgaatgatagcatctataatatctcggagggatataatattaataacttgaaagttaatttccgtggacaaaatgccaatttgaatccgcggattataacaccgattaccggacgtggtctcgtaaaaaagatcattcgtttttgcaaaaacatcgttagcgtaaagggtattcgtaaatcaatttgtatcgaaattaataacggagagttgtttttcgttgccagcgaaaatagctataatgacgataatataaatacccccaaagaaatcgatgacacagtgacctcgaataacaattatgaaaacgatctggatcaagtcatactgaattttaacagtgagtctgctccgggactgtcagacgagaaactgaacttgactatccaaaatgatgcatacattccgaagtatgacagcaacggtacttctgatatagaacagcacgatgtaaatgaactcaatgtgttcttttacctggatgcccaaaaagtgcctgagggagaaaacaatgtaaacctcacttcctcgattgacacagcactgttagaacagccgaaaatatatacctttttctcttcggagtttataaataacgtaaataagcctgtacaagctgccctgttcgtgtcctggatccaacaggtcttagtggacttcactacggaagccaaccaaaagtcgacagtggacaagattgccgatatctctattgtggtcccttacataggtctggcactgaatataggtaatgaagcacaaaaaggaaactttaaagacgccctggaactgttgggcgccggcattcttctcgaatttgaaccggaattgctcatcccgacaatactggtatttacgattaaatcgtttctgggtagttcagataataaaaacaaggtcattaaagctatcaataacgctctgaaagagcgggatgaaaagtggaaagaggtctacagctttatcgtatctaactggatgacgaaaataaatacgcaattcaataaacgtaaagaacagatgtaccaagctttgcagaaccaggtaaatgccatcaagacaatcatagaatcaaagtacaatagttataccttggaagagaaaaatgaattgactaacaaatatgatatcaaacagatagaaaatgaattaaatcagaaagtttcgatcgcaatgaacaatatagatcggtttctgaccgaaagctccattagctatctgatgaaacttataaacgaagtaaaaattaacaaacttcgcgaatatgacgaaaatgtcaagacttacctcttaaattacattatccaacatggttccatcctcggagaaagccagcaagaactgaattccatggtgacagatactctgaataactcgattcccttcaaattgagcagttatacggacgataaaattctgatttcttatttcaataaatttttcaaacggataaaatcgtctagcgttctcaatatgcgttataaaaacgataagtatgttgacaccagtggttatgattctaatattaatattaacggagatgtatataaatatccgacaaataaaaatcagttcggaatctataatgataaacttagtgaagttaatatttcgcaaaacgactatatcatttatgataataaatataaaaacttttcaattagtttctgggtgcgtattccgaattatgataacaagattgtcaatgtaaataacgaatataccatcataaactgcatgcgcgacaataactctggatggaaggtgtctttgaatcataatgaaattatatggactttacaggacaatgcaggaattaaccagaaactggctttcaactatggaaatgctaatggcatcagtgattacattaataaatggatattcgtgactattacaaacgatcgtttgggagattctaaactgtatatcaatggaaatttgatcgatcaaaagtccatattgaatctgggtaacatccatgtgtccgacaacatcttatttaagatcgtgaattgcagttacacccgttacattggcatcagatatttcaacattttcgacaaagaactggatgaaacagaaattcagaccctctattctaatgaacccaatacaaatatattgaaggatttctggggcaattatctgttatacgataaggagtattacttacttaacgttttgaagcccaacaattttatcgaccgccgtaaagattctaccttgagcatcaataacattcgctcaactatcttgcttgccaatcggctgtactcaggcataaaagtgaaaatccaaagagtaaataacagttcgaccaatgataacttggtccgtaagaatgaccaggtgtatatcaacttcgtagctagcaagacgcatcttttcccgttatatgccgataccgctacgactaataaagaaaagacaatcaagatttcttcatcgggaaacagatttaatcaggttgtggtaatgaacagcgtgggaaataactgtaccatgaattttaagaataataatggaaataatattggtttactgggttttaaggccgacacagtagtcgcctcgacctggtattatacacacatgcgtgaccataccaattcaaatggttgtttttggaatttcatttcagaagagcatggctggcaggagaaataa

SEQ ID NO: 11 - DNA, 3759 bp, Artificial Sequence
  mat_peptide (1)...(3756): BoNT/E, B. licheniformis-modified 1
atgccgaaaattaacagctttaattataacgatccggtgaatgatcgcaccattctttatattaaaccgggcggatgtcaggaattctataaatctttcaacataatgaaaaatatctggattatccctgaacggaacgtaattgggacaacgccgcaggatttccaccctccgacatcgctaaaaaacggagactcgtcatattacgatccaaattatctccagtcagacgaggaaaaggatcgcttccttaagattgtcactaagatttttaacaggataaataacaatctttcaggcggaattctgctagaggaactctcgaaggcgaatccgtacctcggaaatgataatacgccggataaccagttccacatcggcgacgcctctgctgtggagatcaaatttagcaatggctcgcaagatatcttgctgccaaacgtcatcattatgggagcggaacctgacttgtttgaaacgaactcctctaatatctcactgcgcaacaattacatgcctagcaaccacgggtttggatcgatcgcgatcgtaacgttttctcccgagtacagctttcgttttaatgacaactcaatgaacgagttcattcaagatccggccctgacgttaatgcacgaactcatccactctttgcatggactgtatggcgccaaaggaatcaccacaaaatatacgattacacagaaacagaaccccctgattacgaacatcagaggtaccaatatcgaggaatttctgacatttggcggtacggacttgaacattataacgtcagcacaaagcaatgacatttataccaatttattggctgattacaagaaaatcgcatcaaaactctcaaaagtccaggtttcaaatcctttattgaatccgtacaaagatgtgttcgaagctaaatacggactagataaggatgccagcggcatatattcggtaaacatcaacaaattcaacgacatcttcaaaaagttgtatagctttactgaatttgacttggcaaccaaatttcaggtcaaatgtagacaaacttacattgggcagtacaaatatttcaaattgtctaatctccttaacgatagtatctataacatcagcgaaggatataatattaacaatctgaaagttaattttagaggccaaaacgccaacctcaatccgcgaatcattaccccgatcacagggcgcggactggttaagaaaatcattcgtttttgcaaaaacattgtgagcgtgaagggcattagaaaatcaatctgcatcgaaattaataacggcgagctttttttcgtcgctagcgaaaattcatacaatgatgacaatattaacacccctaaggagattgatgacacggtcacctcgaacaataactatgaaaacgacttagaccaggttattctgaattttaattcggaatccgcgccgggcctgtcagacgaaaaactcaacctgaccattcagaatgatgcgtacatccccaagtacgactctaatggcacatcggatattgaacaacatgatgtgaatgaacttaatgtgtttttctatctggatgcccagaaagtcccggaaggtgagaacaatgtaaatcttacaagctcaatcgataccgcacttctggagcagccaaaaatctatacgtttttcagctcggaattcataaataacgtcaataaaccggtgcaagctgcccttttcgtcagctggattcaacaggttctcgttgattttacgacagaagccaaccagaaatctactgttgacaaaatagcggacatttcaattgtagttccctacatcggactggcattgaacataggcaatgaggcacaaaaaggcaactttaaagatgctttggaacttctgggtgcggggatactcctggaatttgaacctgaactcctgatcccgacgatcctggtgtttacaattaagtcttttttaggatcctcagataataaaaataaggtgataaaagcgatcaataacgcacttaaagagcgcgacgaaaaatggaaagaagtctacagctttattgtttctaactggatgacaaaaatcaacacgcagtttaacaaaagaaaggaacagatgtaccaagctttacagaaccaagtcaatgcgatcaaaaccatcattgagagtaaatataactcctatactctggaagagaagaacgaactcacaaataagtatgatatcaagcaaattgagaacgaacttaaccagaaagtctcgatcgcaatgaataacattgatcgctttcttacggagtcaagcatcagctatcttatgaagctgatcaacgaggtaaagattaataagctgcgcgaatacgatgaaaatgtgaaaacatatttacttaattatatcattcagcatggttctattttaggcgaaagccaacaggagttaaactccatggtaacagacactttaaacaatagcattccatttaaattatcatcgtacacagacgataaaattcttatttcgtactttaacaaatttttcaagagaatcaaatcctcaagtgttcttaatatgcgctataagaacgataaatatgttgatacaagcggatatgattccaatatcaatattaatggtgatgtctataaatatcctacaaacaaaaatcaatttgggatatacaacgacaaactcagcgaagttaacatctcccagaatgactacatcatttacgacaataaatataagaacttttctatttcgttttgggtcagaatcccgaactacgataataagatcgtgaatgttaacaatgaatatacgatcattaactgcatgcgggataacaattccgggtggaaagtttccttgaatcataatgagatcatttggacgttgcaggataacgccggaattaaccagaaacttgcgtttaactatggcaacgccaacggcatttccgactacatcaataagtggatcttcgtcacgatcacaaatgatcggctcggagactccaagctttatattaacggaaatcttattgatcaaaagagtatcttgaacctgggtaatatccatgtctcagataacatcctgtttaaaattgtcaattgttcgtacactaggtatatcgggattaggtatttcaacatctttgacaaagaattagacgaaacagaaatccaaacgctgtacagcaatgaacctaacacaaacatcctcaaagatttctggggcaattatctcttgtatgacaaagaatattacttactgaacgtcttgaaaccgaataactttatcgaccgtaggaaagactctacgttgagtataaacaatatccggtcaacaatccttttagcgaataggctgtatagcggcataaaagtcaagatccaacgggtgaataacagttcgacgaacgacaacttggtgcgaaaaaacgatcaagtatacatcaatttcgtcgcgagcaaaacgcatttattcccgctttatgccgacacagcaaccacgaataaagaaaaaacgatcaagatatctagctccggtaatcggttcaatcaagttgtggtaatgaatagcgttggaaataactgcaccatgaactttaagaacaataacggcaataacattgggcttcttggctttaaagctgatactgtcgtcgcatccacatggtattatacgcatatgcgtgatcatacgaatagcaatggctgcttttggaactttatttccgaagaacatgggtggcaagaaaaataa

SEQ ID NO: 12 - DNA, 3759 bp, Artificial Sequence
  mat_peptide (1)...(3756): BoNT/E, B. licheniformis-modified 2
atgccgaaaatcaacagctttaactataacgatccggtcaacgatcgcacgatcctgtatatcaaaccgggcggctgccaggaattttataaaagctttaacatcatgaaaaacatctggatcatcccggaacgcaacgtcatcggcacgacgccgcaggattttcatccgccgacgagcctgaaaaacggcgatagcagctattatgatccgaactatctgcagagcgatgaagaaaaagatcgctttctgaaaatcgtcacgaaaatctttaaccgcatcaacaacaacctgagcggcggcatcctgctggaagaactgagcaaagcgaacccgtatctgggcaacgataacacgccggataaccagtttcatatcggcgatgcgagcgcggtcgaaatcaaatttagcaacggcagccaggatatcctgctgccgaacgtcatcatcatgggcgcggaaccggatctgtttgaaacgaacagcagcaacatcagcctgcgcaacaactatatgccgagcaaccatggctttggcagcatcgcgatcgtcacgtttagcccggaatatagctttcgctttaacgataacagcatgaacgaatttatccaggatccggcgctgacgctgatgcatgaactgatccatagcctgcatggcctgtatggcgcgaaaggcatcacgacgaaatatacgatcacgcagaaacagaacccgctgatcacgaacatccgcggcacgaacatcgaagaatttctgacgtttggcggcacggatctgaacatcatcacgagcgcgcagagcaacgatatctatacgaacctgctggcggattataaaaaaatcgcgagcaaactgagcaaagtccaggtcagcaacccgctgctgaacccgtataaagatgtctttgaagcgaaatatggcctggataaagatgcgagcggcatctatagcgtcaacatcaacaaatttaacgatatctttaaaaaactgtatagctttacggaatttgatctggcgacgaaatttcaggtcaaatgccgccagacgtatatcggccagtataaatattttaaactgagcaacctgctgaacgatagcatctataacatcagcgaaggctataacatcaacaacctgaaagtcaactttcgcggccagaacgcgaacctgaacccgcgcatcatcacgccgatcacgggccgcggcctggtcaaaaaaatcatccgcttttgcaaaaacatcgtcagcgtcaaaggcatccgcaaaagcatctgcatcgaaatcaacaacggcgaactgttttttgtcgcgagcgaaaacagctataacgatgataacatcaacacgccgaaagaaatcgatgatacggtcacgagcaacaacaactatgaaaacgatctggatcaggtcatcctgaactttaacagcgaaagcgcgccgggcctgagcgatgaaaaactgaacctgacgatccagaacgatgcgtatatcccgaaatatgatagcaacggcacgagcgatatcgaacagcatgatgtcaacgaactgaacgtctttttttatctggatgcgcagaaagtcccggaaggcgaaaacaacgtcaacctgacgagcagcatcgatacggcgctgctggaacagccgaaaatctatacgttttttagcagcgaatttatcaacaacgtcaacaaaccggtccaggcggcgctgtttgtcagctggatccagcaggtcctggtcgattttacgacggaagcgaaccagaaaagcacggtcgataaaatcgcggatatcagcatcgtcgtcccgtatatcggcctggcgctgaacatcggcaacgaagcgcagaaaggcaactttaaagatgcgctggaactgctgggcgcgggcatcctgctggaatttgaaccggaactgctgatcccgacgatcctggtctttacgatcaaaagctttctgggcagcagcgataacaaaaacaaagtcatcaaagcgatcaacaacgcgctgaaagaacgcgatgaaaaatggaaagaagtctatagctttatcgtcagcaactggatgacgaaaatcaacacgcagtttaacaaacgcaaagaacagatgtatcaggcgctgcagaaccaggtcaacgcgatcaaaacgatcatcgaaagcaaatataacagctatacgctggaagaaaaaaacgaactgacgaacaaatatgatatcaaacagatcgaaaacgaactgaaccagaaagtcagcatcgcgatgaacaacatcgatcgctttctgacggaaagcagcatcagctatctgatgaaactgatcaacgaagtcaaaatcaacaaactgcgcgaatatgatgaaaacgtcaaaacgtatctgctgaactatatcatccagcatggcagcatcctgggcgaaagccagcaggaactgaacagcatggtcacggatacgctgaacaacagcatcccgtttaaactgagcagctatacggatgataaaatcctgatcagctattttaacaaattttttaaacgcatcaaaagcagcagcgtcctgaacatgcgctataaaaacgataaatatgtcgatacgagcggctatgatagcaacatcaacatcaacggcgatgtctataaatatccgacgaacaaaaaccagtttggcatctataacgataaactgagcgaagtcaacatcagccagaacgattatatcatctatgataacaaatataaaaactttagcatcagcttttgggtccgcatcccgaactatgataacaaaatcgtcaacgtcaacaacgaatatacgatcatcaactgcatgcgcgataacaacagcggctggaaagtcagcctgaaccataacgaaatcatctggacgctgcaggataacgcgggcatcaaccagaaactggcgtttaactatggcaacgcgaacggcatcagcgattatatcaacaaatggatctttgtcacgatcacgaacgatcgcctgggcgatagcaaactgtatatcaacggcaacctgatcgatcagaaaagcatcctgaacctgggcaacatccatgtcagcgataacatcctgtttaaaatcgtcaactgcagctatacgcgctatatcggcatccgctattttaacatctttgataaagaactggatgaaacggaaatccagacgctgtatagcaacgaaccgaacacgaacatcctgaaagatttttggggcaactatctgctgtatgataaagaatattatctgctgaacgtcctgaaaccgaacaactttatcgatcgccgcaaagatagcacgctgagcatcaacaacatccgcagcacgatcctgctggcgaaccgcctgtatagcggcatcaaagtcaaaatccagcgcgtcaacaacagcagcacgaacgataacctggtccgcaaaaacgatcaggtctatatcaactttgtcgcgagcaaaacgcatctgtttccgctgtatgcggatacggcgacgacgaacaaagaaaaaacgatcaaaatcagcagcagcggcaaccgctttaaccaggtcgtcgtcatgaacagcgtcggcaacaactgcacgatgaactttaaaaacaacaacggcaacaacatcggcctgctgggctttaaagcggatacggtcgtcgcgagcacgtggtattatacgcatatgcgcgatcatacgaacagcaacggctgcttttggaactttatcagcgaagaacatggctggcaggaaaaataa

SEQ ID NO: 13 - DNA, 3759 bp, Artificial Sequence
  mat_peptide (1)...(3756): BoNT/E, B. licheniformis-modified 3
atgccgaaaatcaactccttcaactacaacgaccctgtcaatgatcgcacgattctctatattaaacctggcgggtgccaggagttctataaatcatttaacatcatgaaaaatatctggatcattccggaaagaaacgtaatcggtacaaccccgcaagacttccatccgcctacgtcattaaaaaatggcgattcatcgtattacgatccgaactacttgcagagcgatgaggaaaaagacagatttcttaagatcgtgacgaaaatcttcaatcggattaacaataacctgagcggcggaattcttttagaagagttatcaaaggctaacccgtacttgggaaacgataatacgccggataatcagttccatatcggagatgccagcgctgtcgaaatcaagttctccaatggctctcaggacatcttgctgccgaacgttattatcatgggcgctgagccggatttgtttgaaacgaatagctcgaatatcagcttgcgcaataactacatgccgtcaaaccatggatttggaagcatcgcgatcgttacattttccccggagtattcatttagatttaacgacaatagcatgaacgaatttattcaggatccggccctgacgttgatgcacgaattgattcactcgctccacggactgtatggcgcgaaagggatcacgacaaagtatacaattacacagaagcagaatccgcttatcacgaacattcgtgggacaaatatcgaagagttcttaacgtttggcggaacagacttgaacatcattacgtcggcacaaagcaacgacatttatacgaaccttctggctgattataagaaaatcgcatcgaagctcagcaaagtccaggtctccaatccgctgttaaatccttacaaagatgtttttgaagcaaaatatggtctggataaagatgcgtcaggaatctattctgtcaacatcaataaatttaatgacattttcaaaaagctctatagctttaccgaatttgaccttgcgaccaaatttcaggtcaaatgccgccaaacatatatcggccaatataaatacttcaaactgtccaacctgcttaacgactcgatctacaatatcagcgagggatacaatattaataacctgaaagtcaactttcgggggcaaaacgcaaacctcaacccgaggatcattacgccgattaccggccgcggcttggttaaaaagatcattcggttttgtaaaaatatcgtgtctgttaaagggatccggaaatccatttgtattgagatcaacaatggtgaattgtttttcgtggcgagcgaaaacagctacaacgatgacaacatcaatacgcctaaggaaattgacgatacggtcacgtctaataacaattacgaaaatgatcttgaccaggtcattttaaactttaactccgaaagcgcccctggactgagcgacgaaaagctcaatttgacgattcaaaatgatgcctatatcccgaaatacgatagcaatgggacatcagacattgaacagcatgatgtcaatgaacttaatgtcttcttttatctggatgcgcaaaaagtcccggagggcgagaacaatgtcaacctgacctccagcatcgatacagcccttctcgagcaaccgaaaatctatacattcttttcttcggaattcatcaacaatgtaaacaaaccggtgcaagctgccctctttgtttcttggattcagcaagtgctcgttgactttacgacagaagccaatcagaaatcaacagttgacaaaatcgcagacatttccatcgtggttccttatattggcctcgcactgaatatcggcaacgaagcccaaaaaggcaactttaaagatgcgctggagctgcttggcgcgggtatcctgcttgaatttgaacctgaactcttaatcccgacgatccttgtctttacaatcaaaagctttctcggatcgtctgataataaaaataaagtaatcaaagcgatcaataacgctcttaaagaaagagacgaaaaatggaaagaggtgtactcatttattgtcagcaattggatgacgaaaattaacacacagtttaacaagcgcaaagaacagatgtaccaggctctccagaaccaggttaatgcgatcaaaaccattatcgaatcaaaatacaattcttatacgctggaagagaagaacgagctgacgaataagtacgatattaaacagatcgaaaacgaactgaaccagaaagtgtcgatcgccatgaataacatcgataggtttcttacagaaagctcgatctcatacttgatgaaactgatcaatgaggtgaagattaataaattgcgcgagtacgatgaaaacgtcaagacctatctgttaaattatattatccaacatggaagcatccttggcgaaagccaacaggaactgaattctatggtgacagatacgttaaataactcaattccgtttaaattgagctcgtacacggacgataagattcttatcagctatttcaataagttctttaagaggattaaatcgagctccgttttgaatatgagatacaaaaacgataagtatgtggatacgagcggctatgattcaaacatcaatatcaatggtgatgtgtacaaatacccgacgaataagaaccaatttgggatttataacgacaagctttcggaagttaatatttcccaaaacgactatattatctacgataacaaatataaaaattttagcatctcattctgggtccggatcccgaattacgacaacaaaattgtaaacgtcaacaatgaatataccattatcaactgcatgagggataataacagcggctggaaggtatccctgaaccacaatgagattatctggacgctgcaagacaacgcaggaattaaccaaaaactggcgtttaattacggcaatgccaatggcatttctgattatatcaacaaatggatcttcgtaacgattacaaacgatcgccttggagattcaaagctctatattaatggtaacctgatcgaccagaagtccattcttaatcttgggaatattcatgtttctgacaacatcctctttaagattgtgaattgttcgtatacgcggtatatcggcatccgttactttaatattttcgacaaagaattagatgaaaccgaaattcaaacactttattcgaatgaacctaatacgaacattcttaaggacttctggggtaattatttgttatatgataaggagtattaccttctgaatgtgctgaaaccgaacaatttcatcgaccgtcgcaaagactccaccctgtcaatcaataacatccgcagcacgattttacttgcgaacaggttatatagcgggatcaaagtcaaaatccaaagagtaaataactcttccacaaatgacaatttagttagaaaaaatgaccaggtttatatcaactttgtagcatcaaagacccatttgtttccgttatatgccgatacagctacgaccaataaggaaaaaacgatcaaaatttcatctagcggaaaccgctttaatcaagtcgttgtgatgaacagcgtcggcaataactgcacaatgaacttcaaaaataacaatggaaacaacatcggattgctcggttttaaagcagataccgtagtcgcatctacgtggtattatacacatatgcgtgatcatacgaactcaaacgggtgcttctggaactttatctcggaagaacacggatggcaggaaaaataa

SEQ ID NO: 14 - DNA, 3759 bp, Artificial Sequence
  mat_peptide (1)...(3756): BoNT/E, B. subtilis-modified 1
atgcccaagataaattcgtttaactacaacgatcctgtgaatgatcgtacgattctttatataaaaccgggaggctgtcaggagttttataaatcgttcaacattatgaaaaatatttggattatcccggagcgcaatgtgataggaacaacgcctcaagactttcatccacctacctcactgaaaaacggtgattcgagttattacgatcccaattatttacagagtgatgaggaaaaagacagatttcttaaaattgttactaaaatttttaaccgtatcaataacaatctgtcaggagggatcttactggaagagcttagtaaagcgaacccgtatcttgggaatgataatactcctgacaatcagttccatattggagacgcttcagcagtcgagataaaattttctaatgggagccaagacattctgcttccgaacgttattatcatgggtgcagaacccgatctgtttgaaaccaatagcagtaatatctctctgagaaataactatatgccgtccaaccacggctttgggagcattgcaattgttacgttttctcctgaatattcttttcgcttcaatgacaatagcatgaacgaatttatccaggacccggcgttaacgttaatgcacgaactcatccatagccttcatggcctctatggagcaaagggaattacaacgaaatatactatcactcaaaagcaaaacccattgataactaacatcagaggcacaaacattgaagagttcctgacattcggcggaaccgatttaaatatcattacaagtgctcagagtaatgatatttatacgaacctgctcgctgattataaaaagattgcatctaagctgtctaaagtccaggtgtctaatcctctactcaacccgtacaaagatgtgtttgaagctaagtatggactagacaaagatgcctccggtatctatagcgtcaacattaacaaatttaatgatatcttcaaaaagttatattcttttacagagtttgacttagcgacaaaatttcaggttaagtgcaggcagacctacattggccagtataaatactttaagctttcaaacctgcttaacgattcgatttacaacatcagcgagggctataatattaacaatttaaaagtaaatttccgaggtcaaaacgcgaaccttaatccgcgcattataacaccgattacaggacggggcctggtgaaaaagatcattagattttgtaagaacattgtatccgtgaaaggaatccggaaaagtatatgtatcgaaatcaacaatggtgagttatttttcgtagcgtctgaaaattcttacaacgatgacaacatcaacacaccaaaagaaattgacgatacagtcacttcaaataacaattatgaaaatgatttagatcaagtcattctgaacttcaattcggaaagcgcgccaggactttcagacgaaaaattaaatctgacgatccaaaatgatgcgtatattccgaagtatgattctaacggcacatcagacatcgaacaacatgatgttaatgaactgaatgtctttttctatctggacgctcaaaaggtcccagagggcgaaaataacgttaatcttacgtcgtcaatagacacagcacttttggagcaaccgaagatttatactttcttttcgagcgaatttattaataacgtgaataaaccggtacaagctgccctatttgtaagctggatccaacaggttttggtggattttacaacggaggccaaccagaagagcacagttgacaaaatcgctgatatatctatcgttgtaccatatatcggacttgcgttgaacatcggcaacgaagcacagaaagggaacttcaaggatgccctagagctcctgggagcagggattttgttagaattcgaacctgagttgcttatacctacaattttagtttttactataaaatcttttttgggctccagcgacaataaaaataaggtcatcaaagcaatcaacaatgctctcaaggagcgggatgaaaaatggaaggaagtctacagcttcattgtttctaattggatgacaaagattaatacccaattcaataaacgaaaagaacaaatgtaccaagcgcttcagaatcaggtaaatgccatcaagactattatcgagagcaaatacaactcctatacacttgaagagaaaaatgaactgacaaataaatatgatattaaacaaattgagaatgaattgaaccagaaggttagcattgcgatgaataacattgacagatttctcacagaaagctcaatctcatatttaatgaaattgattaacgaggtaaaaattaataaattgcgcgaatatgatgaaaatgtcaaaacgtacctcctgaactatatcattcaacatggaagcatcttgggagaatcacaacaggaattgaattcaatggtaaccgatacgttaaataactccatcccgtttaaactgtcatcctacacagatgacaaaatcttgatcagttattttaacaagttctttaagcgaatcaagtcctctagcgttttaaatatgcgctacaagaacgataaatatgttgacacgtcagggtacgattcaaatattaatattaacggggatgtatacaaatatcctactaacaaaaaccaattcggcatatataacgataagttatcggaagtcaatatttcacaaaatgattatattatatatgataataaatataaaaacttttctatcagtttctgggtgagaattccaaactatgataacaaaatcgtgaacgttaataacgaatacacgattatcaattgcatgagagataataacagcggctggaaagtgtcactgaatcacaatgaaatcatttggacgttgcaagacaatgcaggtattaaccaaaagctcgcttttaattatggtaacgccaatggtattagcgactacattaataaatggattttcgtgacaatcaccaatgaccgcctgggagactccaaactgtatatcaatggcaaccttatagaccagaaatcgatactcaatcttggtaacattcatgtgtccgataacattctgtttaaaattgtgaattgctcatatacccggtacatcggcattaggtattttaatatttttgataaagaattggatgaaacagaaatccaaacactgtactcaaatgaaccgaatacgaatattttgaaagatttttggggcaactatttactttatgataaagaatattacttgttaaatgtattaaaaccgaataacttcattgaccgtaggaaagatagcacacttagcataaataacattcgttctacaatacttttagccaatcggctatactccggcattaaagtgaaaattcagcgcgtcaataactccagtacaaacgataaccttgttcgtaaaaatgatcaggtctacatcaattttgtcgcgtctaaaacgcacctctttcctctttatgcagatacagccaccacaaataaagaaaagacgattaaaatctcatcttcaggcaacagatttaatcaggttgtcgttatgaactcagtaggtaataactgtacgatgaattttaaaaacaataacggcaacaatatcgggcttctgggatttaaagccgataccgtggtcgcttcgacgtggtactatactcacatgcgtgaccataccaattccaatggttgcttttggaattttatttctgaggaacatggatggcaggaaaaataa

SEQ ID NO: 15 - DNA, 3759 bp, Artificial Sequence
  mat_peptide (1)...(3756): BoNT/E, B. subtilis-modified 2
atgccgaaaattaatagctttaattataatgatccggttaatgatagaacaattctgtatattaaaccgggcggctgtcaagaattttataaaagctttaatattatgaaaaatatttggattattccggaaagaaatgttattggcacaacaccgcaagattttcatccgccgacaagcctgaaaaatggcgatagcagctattatgatccgaattatctgcaaagcgatgaagaaaaagatagatttctgaaaattgttacaaaaatttttaatagaattaataataatctgagcggcggcattctgctggaagaactgagcaaagcaaatccgtatctgggcaatgataatacaccggataatcaatttcatattggcgatgcaagcgcagttgaaattaaatttagcaatggcagccaagatattctgctgccgaatgttattattatgggcgcagaaccggatctgtttgaaacaaatagcagcaatattagcctgagaaataattatatgccgagcaatcatggctttggcagcattgcaattgttacatttagcccggaatatagctttagatttaatgataatagcatgaatgaatttattcaagatccggcactgacactgatgcatgaactgattcatagcctgcatggcctgtatggcgcaaaaggcattacaacaaaatatacaattacacaaaaacaaaatccgctgattacaaatattagaggcacaaatattgaagaatttctgacatttggcggcacagatctgaatattattacaagcgcacaaagcaatgatatttatacaaatctgctggcagattataaaaaaattgcaagcaaactgagcaaagttcaagttagcaatccgctgctgaatccgtataaagatgtttttgaagcaaaatatggcctggataaagatgcaagcggcatttatagcgttaatattaataaatttaatgatatttttaaaaaactgtatagctttacagaatttgatctggcaacaaaatttcaagttaaatgtagacaaacatatattggccaatataaatattttaaactgagcaatctgctgaatgatagcatttataatattagcgaaggctataatattaataatctgaaagttaattttagaggccaaaatgcaaatctgaatccgagaattattacaccgattacaggcagaggcctggttaaaaaaattattagattttgtaaaaatattgttagcgttaaaggcattagaaaaagcatttgtattgaaattaataatggcgaactgttttttgttgcaagcgaaaatagctataatgatgataatattaatacaccgaaagaaattgatgatacagttacaagcaataataattatgaaaatgatctggatcaagttattctgaattttaatagcgaaagcgcaccgggcctgagcgatgaaaaactgaatctgacaattcaaaatgatgcatatattccgaaatatgatagcaatggcacaagcgatattgaacaacatgatgttaatgaactgaatgtttttttttatctggatgcacaaaaagttccggaaggcgaaaataatgttaatctgacaagcagcattgatacagcactgctggaacaaccgaaaatttatacattttttagcagcgaatttattaataatgttaataaaccggttcaagcagcactgtttgttagctggattcaacaagttctggttgattttacaacagaagcaaatcaaaaaagcacagttgataaaattgcagatattagcattgttgttccgtatattggcctggcactgaatattggcaatgaagcacaaaaaggcaattttaaagatgcactggaactgctgggcgcaggcattctgctggaatttgaaccggaactgctgattccgacaattctggtttttacaattaaaagctttctgggcagcagcgataataaaaataaagttattaaagcaattaataatgcactgaaagaaagagatgaaaaatggaaagaagtttatagctttattgttagcaattggatgacaaaaattaatacacaatttaataaaagaaaagaacaaatgtatcaagcactgcaaaatcaagttaatgcaattaaaacaattattgaaagcaaatataatagctatacactggaagaaaaaaatgaactgacaaataaatatgatattaaacaaattgaaaatgaactgaatcaaaaagttagcattgcaatgaataatattgatagatttctgacagaaagcagcattagctatctgatgaaactgattaatgaagttaaaattaataaactgagagaatatgatgaaaatgttaaaacatatctgctgaattatattattcaacatggcagcattctgggcgaaagccaacaagaactgaatagcatggttacagatacactgaataatagcattccgtttaaactgagcagctatacagatgataaaattctgattagctattttaataaattttttaaaagaattaaaagcagcagcgttctgaatatgagatataaaaatgataaatatgttgatacaagcggctatgatagcaatattaatattaatggcgatgtttataaatatccgacaaataaaaatcaatttggcatttataatgataaactgagcgaagttaatattagccaaaatgattatattatttatgataataaatataaaaattttagcattagcttttgggttagaattccgaattatgataataaaattgttaatgttaataatgaatatacaattattaattgtatgagagataataatagcggctggaaagttagcctgaatcataatgaaattatttggacactgcaagataatgcaggcattaatcaaaaactggcatttaattatggcaatgcaaatggcattagcgattatattaataaatggatttttgttacaattacaaatgatagactgggcgatagcaaactgtatattaatggcaatctgattgatcaaaaaagcattctgaatctgggcaatattcatgttagcgataatattctgtttaaaattgttaattgtagctatacaagatatattggcattagatattttaatatttttgataaagaactggatgaaacagaaattcaaacactgtatagcaatgaaccgaatacaaatattctgaaagatttttggggcaattatctgctgtatgataaagaatattatctgctgaatgttctgaaaccgaataattttattgatagaagaaaagatagcacactgagcattaataatattagaagcacaattctgctggcaaatagactgtatagcggcattaaagttaaaattcaaagagttaataatagcagcacaaatgataatctggttagaaaaaatgatcaagtttatattaattttgttgcaagcaaaacacatctgtttccgctgtatgcagatacagcaacaacaaataaagaaaaaacaattaaaattagcagcagcggcaatagatttaatcaagttgttgttatgaatagcgttggcaataattgtacaatgaattttaaaaataataatggcaataatattggcctgctgggctttaaagcagatacagttgttgcaagcacatggtattatacacatatgagagatcatacaaatagcaatggctgtttttggaattttattagcgaagaacatggctggcaagaaaaataa

SEQ ID NO: 16 - DNA, 3759 bp, Artificial Sequence
  mat_peptide (1)...(3756): BoNT/E, B. subtilis-modified 3
atgccgaagatcaattcatttaactataacgatccggtcaatgatcgtacaattctgtatattaaaccaggcgggtgtcaagagttctataaatcgtttaatattatgaaaaatatctggatcattcctgaacgcaatgttattggtacaacgcctcaagactttcatcctccgacttctcttaaaaacggggactctagctactatgacccgaactacttgcaatcggacgaggaaaaagaccggtttttgaagattgtcaccaaaattttcaatcgcattaacaataacctgtccggcggtatcttactggaagagctttcaaaagccaatccttacctgggaaatgataatacaccggataatcaatttcatattggagatgccagtgctgtcgaaatcaaatttagcaatgggagccaagatattctgttaccgaatgttatcattatgggagctgagccggatctgtttgaaaccaattcttcgaatattagccttcggaataactatatgccgagcaatcacggatttggctccattgcaattgtcacgttcagcccggaatactctttcagatttaacgataattccatgaatgagtttatccaagatccggcgctcacactgatgcacgaactgattcattcgttgcacggcctgtatggagcaaagggaatcacgacaaaatacactattacacaaaaacagaatcctcttattacgaatattcgaggaactaacattgaagagttcttaacctttggaggcacagacttaaatattatcacatcagctcagagtaacgacatctacacgaatctgcttgctgactataagaaaatcgcctctaaattatcaaaagttcaagtatctaatccgttactgaacccgtacaaagacgtgtttgaagccaaatatgggctggacaaagatgcttctggaatttattctgtaaacatcaataagttcaacgacatttttaagaaattatactccttcaccgagttcgatttggcgacgaaatttcaggtcaaatgcaggcaaacgtacattggacaatataagtacttcaaattgtctaatcttctgaacgacagtatctataacatttcagaagggtataatattaacaatttaaaggttaatttccgcggacaaaacgctaatcttaatccaagaatcattacgccgatcacaggtaggggtcttgtgaaaaagattatccgcttttgtaaaaatatcgtatctgtgaaaggtatccgtaaatcaatttgtatcgaaattaataacggagaactgttctttgtagcctcagaaaattcatataacgacgataacatcaacactccgaaggagatcgatgacaccgtcacatctaataacaattatgaaaacgatctcgatcaggttattttaaattttaacagtgaatccgctccaggattgagcgacgaaaagctcaaccttacgattcagaacgatgcctacatcccgaaatatgattcgaacggtacgagtgatattgaacagcatgatgttaacgagctcaatgtctttttctatcttgatgcacagaaagtacctgaaggcgagaacaatgtgaatctcacatccagcattgacacagcgctgctcgaacaaccgaagatttacacgttctttagttcagagtttattaataacgtgaataaacctgtgcaggctgcactttttgtatcttggatccaacaggttctcgtggacttcacaactgaagcaaaccaaaaaagtaccgtcgacaaaattgctgatatttcaatcgtcgtaccatatattgggctggcgttaaatattggtaacgaagcacagaaaggcaattttaaagacgcgcttgaactgcttggtgcaggcatcctccttgaattcgaaccggaattacttatcccaacaatcctggtttttactattaaatcatttctgggtagctcggataacaagaacaaagtgatcaaagcgattaataacgcgcttaaggaacgagatgagaagtggaaagaagtgtatagctttattgttagcaattggatgacaaaaattaatacacagttcaataagcggaaagaacaaatgtaccaagccctgcaaaatcaggttaacgcaatcaaaacgattatcgagtcgaagtacaactcttacacactggaagagaaaaatgagctgacaaacaaatacgacattaaacaaattgagaatgaacttaatcagaaagtgtccattgcgatgaataacattgataggtttctgaccgaaagcagtatttcctatctgatgaaattgattaacgaagttaaaatcaacaaactcagagaatacgatgaaaacgtgaagacatatcttttgaattatattatccaacacggcagtattctgggggaaagccagcaagaattgaacagcatggtaacagacacgttaaataactccattccttttaaacttagctcctatacagatgacaaaattttaattagctactttaacaagtttttcaaaagaattaaatcgagctctgtgcttaatatgcgatacaaaaatgataagtatgtggacacttctggctacgactccaacattaacatcaatggcgatgtctataaatatccaacaaataaaaatcagtttggcatttataacgataaactgtcagaagtcaatatctctcagaacgattatatcatttatgataacaaatataaaaacttctcaatctcattttgggtaagaattccgaactatgataataaaatcgttaacgttaacaatgaatatacaatcattaattgtatgagagataataactcaggatggaaggtaagccttaatcataatgagatcatttggacattgcaagataatgctggcattaaccaaaagctggcatttaattatggtaatgccaacggaattagcgattacattaataaatggatttttgtcactattaccaatgatcgtttgggcgactccaagctttatattaacggcaacttgattgatcagaaatctattctgaatttgggcaatatccatgtatcagataacatcttatttaaaattgtgaattgcagctacactcgctatatcgggattcggtattttaatattttcgataaggaattagatgagacggaaattcagaccctgtattcaaacgagcctaataccaacattctgaaggatttttggggcaattatttattgtatgataaagaatattacttgcttaacgtcctcaagcctaacaattttatcgatcgcagaaaggattctacattaagcatcaacaatattcgctcaacaattttgttagcaaaccgtctttattctggtattaaggttaaaattcagagagttaacaatagttcaactaacgataacttagttcggaaaaatgaccaggtgtatatcaattttgttgccagcaaaacacatttgttcccattatatgcggatactgcgacgacaaataaagaaaaaacaatcaagatctcctcgtcaggaaaccgttttaatcaggtcgttgtgatgaattcagtcggcaataactgcaccatgaattttaaaaacaataacggaaataacattggactcttaggctttaaagcggacacggtcgtagcatcgacgtggtattatacgcatatgcgtgatcacacgaattcaaatggctgcttttggaattttatttctgaagagcatgggtggcaagaaaaataa

SEQ ID NO: 17 - DNA, 3759 bp, Artificial Sequence
  mat_peptide (1)...(3756): BoNT/E, C. difficile-modified 1
atgcctaaaattaattcttttaattataatgatcctgttaacgatagaacaatattatatattaaaccgggtgggtgtcaggagttttataaatcttttaatataatgaagaatatatggataattcctgaaagaaatgttataggaacgacacctcaagattttcacccacctacgagtcttaaaaatggggactctagttattacgatccaaattatctacagtcagatgaagagaaggacagatttttaaaaattgttactaaaatttttaatcgtattaataacaatttatcaggaggtattttacttgaagagctatctaaagcaaacccatatctggggaatgataatacaccagataatcaatttcatattggagacgctagtgccgtagaaattaaatttagcaatggttctcaggatatcttgctgccaaatgtaattatcatgggagctgaaccagatttatttgagaccaattcttccaatataagccttagaaacaattatatgccttctaatcacggttttggatcaattgcaattgttacattttcgccagaatattcttttagatttaatgacaactctatgaacgaatttatacaagatcctgctttaacactaatgcacgagttaatacactctcttcatggattgtatggtgcaaaaggtattactacaaagtatactattacacaaaaacagaatcctctaataacaaacatacgtggtacaaatatagaagagtttctaacatttggaggtacagatcttaatataattacttcagctcaatcaaacgatatatatacgaatttgttagctgattacaaaaagattgctagcaaattaagcaaagtacaagtctcaaatccattacttaatccatataaagatgtatttgaagctaaatatggtttagataaggatgcatcaggtatatattcagtgaacataaacaaatttaatgatatatttaaaaagctctattcattcactgaatttgacttagcaacaaaatttcaagtaaaatgtagacaaacttatattggacagtataagtattttaagctttctaacttactaaatgattctatttataatatatctgaaggatataacattaataaccttaaagttaattttagaggccaaaatgctaacttgaatcctagaattataactccaataactggaagaggattagtaaaaaagattataagattttgtaaaaatattgtatcagttaaaggaattagaaaatcaatttgcatcgaaatcaataacggagaactatttttcgtagcatccgaaaactcatacaatgatgacaacataaatacacctaaagaaatagatgacactgtaacaagtaataacaattatgaaaatgatctagatcaagtaatactaaattttaattctgaatcagctccaggtttatcagatgaaaaattaaatctaacaatacagaatgacgcttatatcccaaaatatgattcaaatggtactagtgatatagagcaacatgatgtaaatgaattaaatgtgtttttctatttagatgctcagaaggtacctgaaggagaaaacaatgttaatctgactagctcaattgataccgcattactcgaacaacctaaaatttatacatttttcagttcagaatttataaataacgttaataaacctgtacaagcagcgttatttgtatcgtggattcaacaggtattagtcgattttactacagaagcaaatcaaaaatcaactgtagataaaatagccgatataagtattgtggtaccatatataggattagcactaaacataggcaatgaagctcaaaaagggaattttaaagatgcattagagctgttaggcgctggaattttattggagtttgagccagaattacttatacctactatcttagttttcacaataaaaagtttcttaggaagttctgacaataaaaataaagttatcaaagcaataaacaatgctttaaaagaaagagacgaaaaatggaaggaagtttatagttttatagtatccaattggatgactaaaattaatacacaatttaataagcgcaaggaacaaatgtaccaagcacttcaaaatcaagtgaatgctattaaaactataattgagtctaaatataatagttatacattggaagagaaaaatgaattaacaaataagtatgatattaaacaaatagaaaatgaattaaatcaaaaagttagtattgctatgaataacatagatagattccttactgaatctagtatatcatatttaatgaaattaataaatgaagtaaagattaataaattaagagaatatgacgaaaatgtaaaaacttatttattgaattatataatccaacatggttctattttgggtgaaagtcaacaagaattaaattctatggtaactgatactttaaataacagtataccatttaaattaagttcttatacagatgacaaaattttaatttcttattttaataaatttttcaaaagaataaaatctagctcagttcttaatatgaggtataaaaatgataagtatgttgatacctctggatacgattcaaatattaatattaacggtgatgtgtataaatatccaacaaataaaaatcaattcggtatatataatgataaattaagtgaagttaatataagtcaaaatgattatatcatatacgataataaatataaaaattttagtatatctttttgggtgagaatacccaattatgataacaaaatagttaatgtaaataacgagtatactattataaactgtatgcgagataataactctggatggaaagtttctttgaatcataatgaaataatctggactttacaagataatgcaggaattaatcaaaaattagcctttaattatggtaatgcaaatggaattagtgattacattaataaatggatatttgttacgataactaacgataggttaggtgatagtaaactttacataaatggaaatttaattgatcaaaagtcaattttaaatcttggtaatatacatgttagtgataatatattatttaaaatagtaaattgttcatatactagatatataggaataagatattttaatatatttgataaagaacttgatgaaacagaaattcaaacactttactcaaatgaacctaatactaatattttaaaagatttttggggaaattatttattgtacgataaagaatactatcttttaaatgtactaaaaccaaataattttatagatagacgaaaagatagtactttatcaataaataatataagaagtacaatattattggcaaatcgtttatacagtggaataaaggtaaagatacaacgtgttaataattcatctactaatgataatttagttcggaaaaatgatcaagtatatataaattttgtagcatctaaaactcatttattccctttatatgctgatactgcaactaccaataaagaaaaaactataaagataagttctagtggtaatagatttaatcaagtagttgtcatgaattctgttggcaataattgtactatgaatttcaaaaataataatggtaataatattggtttattaggatttaaggctgatacagttgttgcaagtacttggtattatacacatatgagagatcatactaatagtaatggttgcttttggaattttatatcagaagaacatggatggcaagaaaaataa

SEQ ID NO: 18 - DNA, 3759 bp, Artificial Sequence
  mat_peptide (1)...(3756): BoNT/E, C. difficile-modified 2
atgccaaaaataaattcttttaattataatgatccagtaaatgatagaactatattatatataaaaccaggaggatgtcaagaattttataaatcttttaatataatgaaaaatatatggataataccagaaagaaatgtaataggaactactccacaagattttcatccaccaacttctttaaaaaatggagattcttcttattatgatccaaattatttacaatctgatgaagaaaaagatagatttttaaaaatagtaactaaaatatttaatagaataaataataatttatctggaggaatattattagaagaattatctaaagcaaatccatatttaggaaatgataatactccagataatcaatttcatataggagatgcatctgcagtagaaataaaattttctaatggatctcaagatatattattaccaaatgtaataataatgggagcagaaccagatttatttgaaactaattcttctaatatatctttaagaaataattatatgccatctaatcatggatttggatctatagcaatagtaactttttctccagaatattcttttagatttaatgataattctatgaatgaatttatacaagatccagcattaactttaatgcatgaattaatacattctttacatggattatatggagcaaaaggaataactactaaatatactataactcaaaaacaaaatccattaataactaatataagaggaactaatatagaagaatttttaacttttggaggaactgatttaaatataataacttctgcacaatctaatgatatatatactaatttattagcagattataaaaaaatagcatctaaattatctaaagtacaagtatctaatccattattaaatccatataaagatgtatttgaagcaaaatatggattagataaagatgcatctggaatatattctgtaaatataaataaatttaatgatatatttaaaaaattatattcttttactgaatttgatttagcaactaaatttcaagtaaaatgtagacaaacttatataggacaatataaatattttaaattatctaatttattaaatgattctatatataatatatctgaaggatataatataaataatttaaaagtaaattttagaggacaaaatgcaaatttaaatccaagaataataactccaataactggaagaggattagtaaaaaaaataataagattttgtaaaaatatagtatctgtaaaaggaataagaaaatctatatgtatagaaataaataatggagaattattttttgtagcatctgaaaattcttataatgatgataatataaatactccaaaagaaatagatgatactgtaacttctaataataattatgaaaatgatttagatcaagtaatattaaattttaattctgaatctgcaccaggattatctgatgaaaaattaaatttaactatacaaaatgatgcatatataccaaaatatgattctaatggaacttctgatatagaacaacatgatgtaaatgaattaaatgtatttttttatttagatgcacaaaaagtaccagaaggagaaaataatgtaaatttaacttcttctatagatactgcattattagaacaaccaaaaatatatacttttttttcttctgaatttataaataatgtaaataaaccagtacaagcagcattatttgtatcttggatacaacaagtattagtagattttactactgaagcaaatcaaaaatctactgtagataaaatagcagatatatctatagtagtaccatatataggattagcattaaatataggaaatgaagcacaaaaaggaaattttaaagatgcattagaattattaggagcaggaatattattagaatttgaaccagaattattaataccaactatattagtatttactataaaatcttttttaggatcttctgataataaaaataaagtaataaaagcaataaataatgcattaaaagaaagagatgaaaaatggaaagaagtatattcttttatagtatctaattggatgactaaaataaatactcaatttaataaaagaaaagaacaaatgtatcaagcattacaaaatcaagtaaatgcaataaaaactataatagaatctaaatataattcttatactttagaagaaaaaaatgaattaactaataaatatgatataaaacaaatagaaaatgaattaaatcaaaaagtatctatagcaatgaataatatagatagatttttaactgaatcttctatatcttatttaatgaaattaataaatgaagtaaaaataaataaattaagagaatatgatgaaaatgtaaaaacttatttattaaattatataatacaacatggatctatattaggagaatctcaacaagaattaaattctatggtaactgatactttaaataattctataccatttaaattatcttcttatactgatgataaaatattaatatcttattttaataaattttttaaaagaataaaatcttcttctgtattaaatatgagatataaaaatgataaatatgtagatacttctggatatgattctaatataaatataaatggagatgtatataaatatccaactaataaaaatcaatttggaatatataatgataaattatctgaagtaaatatatctcaaaatgattatataatatatgataataaatataaaaatttttctatatctttttgggtaagaataccaaattatgataataaaatagtaaatgtaaataatgaatatactataataaattgtatgagagataataattctggatggaaagtatctttaaatcataatgaaataatatggactttacaagataatgcaggaataaatcaaaaattagcatttaattatggaaatgcaaatggaatatctgattatataaataaatggatatttgtaactataactaatgatagattaggagattctaaattatatataaatggaaatttaatagatcaaaaatctatattaaatttaggaaatatacatgtatctgataatatattatttaaaatagtaaattgttcttatactagatatataggaataagatattttaatatatttgataaagaattagatgaaactgaaatacaaactttatattctaatgaaccaaatactaatatattaaaagatttttggggaaattatttattatatgataaagaatattatttattaaatgtattaaaaccaaataattttatagatagaagaaaagattctactttatctataaataatataagatctactatattattagcaaatagattatattctggaataaaagtaaaaatacaaagagtaaataattcttctactaatgataatttagtaagaaaaaatgatcaagtatatataaattttgtagcatctaaaactcatttatttccattatatgcagatactgcaactactaataaagaaaaaactataaaaatatcttcttctggaaatagatttaatcaagtagtagtaatgaattctgtaggaaataattgtactatgaattttaaaaataataatggaaataatataggattattaggatttaaagcagatactgtagtagcatctacttggtattatactcatatgagagatcatactaattctaatggatgtttttggaattttatatctgaagaacatggatggcaagaaaaataa

SEQ ID NO: 19 - DNA, 3759 bp, Artificial Sequence
  mat_peptide (1)...(3756): BoNT/E, C. difficile-modified 3
atgcctaaaattaattcttttaattataatgatccagtaaatgatagaacaatactatatattaaacctggtggatgtcaagaattttataaatcatttaatattatgaaaaatatatggataattcctgaaagaaatgttataggaactacaccacaagattttcatcctccaactagtcttaaaaatggtgattcttcatattatgatccaaattatctacaatctgatgaagaaaaagatagatttttaaaaattgtaactaaaatatttaatagaataaataataatttatctggtggaattttactagaagaattatcaaaagctaatccatatttaggaaatgataatactcctgataatcaatttcatattggagatgcaagtgctgtagaaataaaattttctaatggtagtcaagatatattattgccaaatgtaattataatgggtgcagaaccagatttatttgaaacaaatagttcaaatattagtttaagaaataattatatgccatctaatcatggatttggttctatagctattgtaacttttagtccagaatatagttttagatttaatgataattctatgaatgaatttatacaagatcctgctttaacattaatgcacgaattaatacattctctacatggtttatatggtgctaaaggaataactacaaaatatactattactcaaaaacaaaatcctctaattacaaatattagaggaactaatatagaagaatttttaacttttggtggaactgatctaaatattataacttcagcacaatctaatgatatatatactaatttacttgctgattataaaaaaattgcttctaaactttctaaagttcaagtttcaaatccattactaaatccatataaagatgtatttgaagctaaatatggacttgataaagatgcaagtggaatttattcagttaatattaataaatttaatgatatttttaaaaaattatattcatttacagaatttgatttagcaactaaatttcaagttaaatgtagacaaacatatattggtcaatataaatattttaaattaagtaatcttttaaatgattctatatataatatatcagaaggttataatataaataatcttaaagtaaattttagaggtcaaaatgcaaatttgaatcctcgtataattactcctataacaggtagaggattagttaaaaaaataattagattttgtaaaaatatagtaagtgtaaaaggtattagaaaaagtatatgtattgaaattaataatggagaattattttttgtagcatctgaaaattcatataatgatgataatataaatactcctaaagaaatagatgatactgtaacttcaaataataattatgaaaatgatttagatcaagttatattaaattttaatagtgaatctgctcctggactttctgatgaaaaattaaatttaactattcaaaatgatgcatatattccaaaatatgattcaaatggtacttctgatatagaacaacacgatgtaaatgaattaaatgtatttttttatttagatgcacaaaaagtaccagaaggagaaaataatgttaatttaacttcttcaatagatactgcattgttagaacaaccaaaaatatatacatttttttcatctgaatttataaataatgttaataaacctgtacaagctgcactatttgttagttggattcaacaagttttagtagattttacaactgaagcaaatcaaaaatcaactgtagataaaatagcagatatttctatagtagttccttatattggtttggcattgaatattggaaatgaagcacaaaaaggaaattttaaagatgcattggaattactaggagctggaatacttttagaatttgaacctgaattgcttataccaactattttagtatttacaataaaatcttttcttggatcaagtgataataaaaataaagtaataaaagcaataaataatgcattaaaagaaagagatgaaaaatggaaagaagtttatagttttatagtttctaattggatgactaaaataaatactcaatttaataaaagaaaagaacaaatgtatcaagctttacaaaatcaagtaaatgctataaaaacaataattgaatcaaaatataattcttatactttagaagaaaaaaatgaattaacaaataaatatgatataaaacaaatagaaaatgaattaaatcaaaaagtaagtatagctatgaataatatagatagatttttaactgaatcttcaatttcttatcttatgaaacttataaatgaagtaaaaattaataaattgagagaatatgatgaaaatgtaaaaacttatttacttaattatattatacaacatggatctatattaggtgaatctcaacaagaattaaattcaatggttactgatacattaaataattcaattccttttaaattatcttcatatactgatgataaaatacttatatcttattttaataaattttttaaacgtattaaatcttcatctgttttaaatatgagatataaaaatgataaatatgttgatacaagtggatatgatagtaatataaatataaatggagatgtatataaatatccaacaaataaaaatcaatttggaatttataatgataaattatcagaagttaatatatctcaaaatgattatattatatatgataataaatataaaaatttttcaataagtttttgggtaagaattccaaattatgataataaaatagtaaatgtaaataatgaatatacaataattaattgtatgcgtgataataatagtggttggaaagttagtcttaatcacaatgaaataatttggactcttcaagataatgcaggaattaatcaaaaattagcatttaattatggtaatgctaatggaatatctgattatattaataaatggatatttgttacaataactaatgatagattaggagatagtaaattatatattaatggaaatttaatagatcaaaaaagtattttaaatttgggaaatatacatgtaagtgataatatattatttaaaatagttaattgttcttatactagatatataggaattagatattttaatatatttgataaagaactagatgaaacagaaattcaaactttatattctaatgaaccaaatactaatatattaaaagatttttggggtaattatctattatatgataaagaatattatcttttgaatgtattgaaacctaataattttatagatcgtagaaaagatagtacattatctataaataatataagatctactatactattagctaatagattatatagtggaataaaagttaaaatacaaagagtaaataattcttcaactaatgataatttagttagaaaaaatgatcaagtttatattaattttgtagcaagtaaaacacatttatttccattatatgctgatacagcaacaactaataaagaaaaaactataaaaataagttcttcaggtaatagatttaatcaagtagttgtaatgaattctgtaggtaataattgtacaatgaattttaaaaataataatggaaataatataggattattaggatttaaagcagatactgtagtagcttcaacatggtattatacacatatgagagatcacacaaatagtaatggatgtttttggaattttatatctgaagaacatggttggcaagaaaaataa

SEQ ID NO: 20 - DNA, 3759 bp, Artificial Sequence
  mat_peptide (1)...(3756): BoNT/E, C. perfringens-modified 1
atgccaaaaattaattcttttaactataatgatccagttaatgataggactatcttatatataaaacctggaggttgccaagaattttataaaagttttaacataatgaaaaatatttggataattcctgaaagaaatgttataggaacaacccctcaagattttcatcctccaacaagtcttaagaatggagattcaagctattacgacccaaattatttacaaagtgatgaagagaaggatagatttttaaagatagttactaaaatatttaacagaattaataacaatttaagcggcggaattcttttagaggaattatctaaagcaaatccatatttaggtaatgataatactcctgataatcaatttcacattggtgatgctagcgctgtagagataaaattttctaatggaagtcaagatatacttctaccaaacgtaattataatgggtgctgaaccagatttattcgaaacaaattcatctaatatatcattaagaaataactatatgccatccaatcacggattcggaagtatagctatagttacattttcacctgaatattcatttagatttaatgataacagtatgaatgaatttatacaagatcctgctttaacattaatgcatgagttaatacattcattacatggattatatggagcaaaaggaattactaccaaatatacaataacacaaaagcaaaaccctttaataacaaatataagaggaactaatattgaagagtttttaacttttggaggtacagatttaaatattataacatctgcacaatctaacgatatatatactaatcttttagctgattataaaaagattgcatcaaaactgagcaaagttcaagttagtaatccacttttaaatccatataaggatgtgtttgaagctaaatatgggttagataaagatgcatcaggaatttacagcgttaatataaataagtttaatgatatattcaaaaagttatatagtttcacagaatttgatttagcaacaaaatttcaagtaaaatgtagacagacttatattggacaatataaatattttaaattaagtaatttacttaatgatagtatctacaatatatcagaaggatataacattaataacttgaaagtgaattttagaggccaaaatgctaatttaaatccaagaataattactcctattactggaagaggtttagtaaaaaagattataagattttgtaaaaatatagtgtcagttaaaggaataagaaagagtatttgtatagaaataaataacggagaactattctttgttgcctcagaaaatagttacaatgatgacaatattaacactccaaaagagatagatgacacagtaacaagcaataacaattatgaaaacgatttagaccaagttatacttaattttaattctgaatcagctcctgggctatctgatgagaaacttaatttaactatacaaaacgatgcatatataccaaaatacgatagtaatggtacatcagatatcgaacaacatgatgtaaatgaattaaatgtatttttctaccttgatgcccaaaaagttcctgagggagaaaataacgttaatttaacttcttccatagatacagcattactagaacaacctaagatatacacttttttcagttctgagtttataaataacgttaataaacctgtacaagctgcactttttgtatcttggattcaacaggttttagtagattttactacagaagcaaaccaaaaaagtactgtagataaaattgctgatatatcaattgtagttccatatattgggcttgctttaaatatcggaaatgaagcacaaaaaggaaattttaaagatgctttagaattactaggagctggaatattacttgagtttgaaccagagttacttataccaacaatattagtatttactattaagagttttttaggttcttcagataataaaaataaagttataaaagctattaataacgctcttaaagaaagagatgaaaagtggaaagaagtttatagttttatagtatcaaattggatgacaaagataaatactcaatttaataagagaaaagagcaaatgtatcaggctctacaaaaccaagtaaatgctataaaaacgataatcgaatctaagtataacagttatacattagaggaaaagaatgaactaactaataaatatgatataaagcaaatagaaaacgaattaaatcaaaaggtttcaatagcaatgaataacattgatagatttttaactgaatcaagcatatcatatttaatgaagttaataaatgaagttaaaattaataaattaagggaatatgatgaaaatgtaaagacttaccttttaaattacataattcaacatggttcaattttaggtgaatcacaacaggaattaaattctatggtcacagacactttgaataactctataccatttaagttatcaagttatactgatgacaagatattaataagttattttaataaatttttcaaaagaataaaatcttcatctgttcttaatatgagatataagaatgataaatatgtagatacatctggttatgatagtaatattaatataaatggtgatgtatataaatatcccactaataaaaaccagtttggaatttataatgataaattatcagaagtgaacatatctcaaaatgattacataatttatgataataaatataaaaattttagtattagtttctgggttagaatacctaattatgacaataaaatagtaaatgtaaataacgaatatacaataattaattgtatgagagataataactctgggtggaaagtttcattaaatcataatgaaataatttggacattacaagataatgctggaataaatcaaaaactagcctttaattatggaaatgctaatggcatatctgattatataaataagtggatatttgttactattactaatgatagattaggagattcaaagttatatataaatggaaatttaatagatcaaaaaagtattttaaatttaggtaatatacatgtttccgataatatactttttaagatagttaattgttcttacacaagatatataggtataagatatttcaatatatttgataaagaactagatgagactgaaattcagactttatattccaatgaaccaaatactaatatattaaaagatttttggggtaattatcttttatatgataaagaatattatttattgaatgttttgaaaccaaataattttatagacaggagaaaagattcaactttatcaataaataatattagaagtactatacttttagcaaatagattatatagtggaataaaagtaaagatacaaagagttaataattcaagcactaatgataatcttgttagaaaaaatgaccaagtatatattaattttgtagcttcgaaaacccatttatttcctttatatgctgatacagcgacaactaataaagaaaagacaataaaaatttctagttctggtaatagatttaatcaagttgtagttatgaattcagtaggaaataattgtactatgaatttcaaaaataataatggaaataatatcggattacttgggtttaaagcagataccgttgttgcaagtacttggtattatacacatatgcgtgatcacactaattcaaatggatgcttttggaatttcatttctgaggaacatggatggcaagaaaaataa

SEQ ID NO: 21 - DNA, 3759 bp, Artificial Sequence
  mat_peptide (1)...(3756): BoNT/E, C. perfringens-modified 2
atgccaaaaataaattcatttaattataatgatccagttaatgatagaactatattatatataaaaccaggaggatgtcaagaattttataaatcatttaatataatgaaaaatatatggataataccagaaagaaatgttataggaactactccacaagattttcatccaccaacttcattaaaaaatggagattcatcatattatgatccaaattatttacaatcagatgaagaaaaagatagatttttaaaaatagttactaaaatatttaatagaataaataataatttatcaggaggaatattattagaagaattatcaaaagctaatccatatttaggaaatgataatactccagataatcaatttcatataggagatgcttcagctgttgaaataaaattttcaaatggatcacaagatatattattaccaaatgttataataatgggagctgaaccagatttatttgaaactaattcatcaaatatatcattaagaaataattatatgccatcaaatcatggatttggatcaatagctatagttactttttcaccagaatattcatttagatttaatgataattcaatgaatgaatttatacaagatccagctttaactttaatgcatgaattaatacattcattacatggattatatggagctaaaggaataactactaaatatactataactcaaaaacaaaatccattaataactaatataagaggaactaatatagaagaatttttaacttttggaggaactgatttaaatataataacttcagctcaatcaaatgatatatatactaatttattagctgattataaaaaaatagcttcaaaattatcaaaagttcaagtttcaaatccattattaaatccatataaagatgtttttgaagctaaatatggattagataaagatgcttcaggaatatattcagttaatataaataaatttaatgatatatttaaaaaattatattcatttactgaatttgatttagctactaaatttcaagttaaatgtagacaaacttatataggacaatataaatattttaaattatcaaatttattaaatgattcaatatataatatatcagaaggatataatataaataatttaaaagttaattttagaggacaaaatgctaatttaaatccaagaataataactccaataactggaagaggattagttaaaaaaataataagattttgtaaaaatatagtttcagttaaaggaataagaaaatcaatatgtatagaaataaataatggagaattattttttgttgcttcagaaaattcatataatgatgataatataaatactccaaaagaaatagatgatactgttacttcaaataataattatgaaaatgatttagatcaagttatattaaattttaattcagaatcagctccaggattatcagatgaaaaattaaatttaactatacaaaatgatgcttatataccaaaatatgattcaaatggaacttcagatatagaacaacatgatgttaatgaattaaatgtttttttttatttagatgctcaaaaagttccagaaggagaaaataatgttaatttaacttcatcaatagatactgctttattagaacaaccaaaaatatatacttttttttcatcagaatttataaataatgttaataaaccagttcaagctgctttatttgtttcatggatacaacaagttttagttgattttactactgaagctaatcaaaaatcaactgttgataaaatagctgatatatcaatagttgttccatatataggattagctttaaatataggaaatgaagctcaaaaaggaaattttaaagatgctttagaattattaggagctggaatattattagaatttgaaccagaattattaataccaactatattagtttttactataaaatcatttttaggatcatcagataataaaaataaagttataaaagctataaataatgctttaaaagaaagagatgaaaaatggaaagaagtttattcatttatagtttcaaattggatgactaaaataaatactcaatttaataaaagaaaagaacaaatgtatcaagctttacaaaatcaagttaatgctataaaaactataatagaatcaaaatataattcatatactttagaagaaaaaaatgaattaactaataaatatgatataaaacaaatagaaaatgaattaaatcaaaaagtttcaatagctatgaataatatagatagatttttaactgaatcatcaatatcatatttaatgaaattaataaatgaagttaaaataaataaattaagagaatatgatgaaaatgttaaaacttatttattaaattatataatacaacatggatcaatattaggagaatcacaacaagaattaaattcaatggttactgatactttaaataattcaataccatttaaattatcatcatatactgatgataaaatattaatatcatattttaataaattttttaaaagaataaaatcatcatcagttttaaatatgagatataaaaatgataaatatgttgatacttcaggatatgattcaaatataaatataaatggagatgtttataaatatccaactaataaaaatcaatttggaatatataatgataaattatcagaagttaatatatcacaaaatgattatataatatatgataataaatataaaaatttttcaatatcattttgggttagaataccaaattatgataataaaatagttaatgttaataatgaatatactataataaattgtatgagagataataattcaggatggaaagtttcattaaatcataatgaaataatatggactttacaagataatgctggaataaatcaaaaattagcttttaattatggaaatgctaatggaatatcagattatataaataaatggatatttgttactataactaatgatagattaggagattcaaaattatatataaatggaaatttaatagatcaaaaatcaatattaaatttaggaaatatacatgtttcagataatatattatttaaaatagttaattgttcatatactagatatataggaataagatattttaatatatttgataaagaattagatgaaactgaaatacaaactttatattcaaatgaaccaaatactaatatattaaaagatttttggggaaattatttattatatgataaagaatattatttattaaatgttttaaaaccaaataattttatagatagaagaaaagattcaactttatcaataaataatataagatcaactatattattagctaatagattatattcaggaataaaagttaaaatacaaagagttaataattcatcaactaatgataatttagttagaaaaaatgatcaagtttatataaattttgttgcttcaaaaactcatttatttccattatatgctgatactgctactactaataaagaaaaaactataaaaatatcatcatcaggaaatagatttaatcaagttgttgttatgaattcagttggaaataattgtactatgaattttaaaaataataatggaaataatataggattattaggatttaaagctgatactgttgttgcttcaacttggtattatactcatatgagagatcatactaattcaaatggatgtttttggaattttatatcagaagaacatggatggcaagaaaaataa

SEQ ID NO: 22 - DNA, 3759 bp, Artificial Sequence
  mat_peptide (1)...(3756): BoNT/E, C. perfringens-modified 3
atgcctaaaataaattcatttaattataatgatcctgttaatgatagaacaatactatatataaaaccaggaggttgtcaagaattttataagagttttaatattatgaaaaatatatggataattccagaaagaaatgtaataggaactacacctcaagattttcatccacctacatcattaaaaaatggagattcaagttattatgatcctaattatttacaaagtgatgaagaaaaagatagatttctaaaaattgttactaaaatttttaatagaataaataataatttaagtggtggaattttactagaagaattatcaaaagctaatccatatttaggaaatgataatactccagataatcaatttcatataggagatgctagcgctgtagaaattaaattttcaaatggatcacaagatatattacttcctaatgtaataattatgggtgctgaacctgatttatttgaaactaattcatctaatatttctttaaggaataattatatgccaagcaatcatggttttggatcaatagctatagtaacattttcaccagaatattcttttagatttaatgataattcaatgaatgaatttatacaagatccagctttaactcttatgcatgaattaattcattcattacatggactttatggtgctaaaggaataacaactaaatatacaataacacaaaaacaaaatccacttattacaaatattagaggtacaaatatagaagaatttttaacttttggaggtactgatttaaatataattacaagtgctcaatcaaatgatatatatacaaatttacttgctgattataaaaagatagcatcaaagcttagtaaagttcaagtttcaaatccattactaaatccatataaagatgtatttgaagcaaaatatggtttagataaagatgcaagcggaatatatagcgtaaatattaataagtttaatgatatttttaaaaagttatatagttttactgaatttgatcttgctactaaatttcaagttaaatgtaggcaaacttatataggacaatataagtattttaaacttagtaatttacttaatgattcaatatataatatatcagaaggttataatataaataatttaaaagttaattttagaggacaaaatgcaaatttaaatccaagaataattactccaataacaggaagaggtttagttaaaaagataattagattttgtaaaaatatagtatctgtaaaaggaataagaaaatctatatgtatagaaataaataatggagaacttttttttgttgcttctgaaaatagttataatgatgataatataaatacacctaaagaaatagatgatactgtaacttcaaataataattatgaaaatgatttagatcaagtaattttaaattttaatagtgaatcagctcctggattaagcgatgaaaaattaaatttaacaatacaaaatgatgcatatataccaaaatatgatagtaatggaacttcagatatagaacaacatgatgttaatgaattaaatgtttttttttatttagatgctcaaaaagtacctgaaggagaaaataatgttaatcttactagttctatagatactgcactattagaacaaccaaaaatatatacttttttttcatctgaatttattaataatgttaataaaccagttcaagcagctttatttgtttcttggatacaacaagttttagttgattttacaactgaagcaaatcaaaagagtactgttgataagattgctgatataagtattgtagttccttatataggtttagctttaaatataggaaatgaagctcaaaaaggaaattttaaagatgctttagaattacttggagctggaatattacttgaatttgaaccagaattacttatacctacaattctagtttttactattaagagttttttaggatcaagcgataataagaataaagttataaaggcaattaataatgctttaaaagaaagagatgaaaaatggaaggaagtatattcatttattgtttcaaattggatgactaaaataaatactcaatttaataaaagaaaggaacaaatgtatcaagctttacaaaatcaagttaatgcaataaaaactataattgaaagcaagtataattcatatacacttgaagaaaaaaatgaattaactaataaatatgatataaagcaaatagaaaatgaattaaatcaaaaggtaagtatagcaatgaataatatagatagatttttaactgaaagttcaatatcttatttaatgaagttaataaatgaagtaaaaataaataaattaagagaatatgatgaaaatgttaaaacatatcttttaaattatattatacaacatggaagtattttaggtgaatcacaacaagaattaaatagtatggttacagatactttaaataatagtattccttttaaattaagttcttatactgatgataaaatattaatatcatattttaataaattttttaaaagaataaaatcatcttcagtattaaatatgagatataagaatgataaatatgttgatacaagtggttatgattctaatataaatataaatggtgatgtttataaatatcctacaaataaaaatcaatttggaatttataatgataagttatctgaagttaatatttctcaaaatgattatattatatatgataataagtataaaaatttttcaataagtttttgggttagaataccaaattatgataataaaattgtaaatgttaataatgaatatactataattaattgtatgagagataataatagcggatggaaggtttctctaaatcataatgaaattatatggacattacaagataatgctggaataaatcaaaaattagcatttaattatggaaatgcaaatggaatatctgattatataaataagtggatatttgttactataacaaatgatagactaggtgattctaagttatatataaatggaaatttaatagatcaaaaatctatattaaatttaggaaatatacatgtatcagataatatattatttaaaatagttaattgtagttatactaggtatataggaattagatattttaatatttttgataaggaattagatgaaactgaaatacaaactctttattcaaatgaaccaaatacaaatattctaaaggatttttggggaaattatcttttatatgataaagaatattatttacttaatgttttaaaaccaaataattttattgatagaagaaaggattctacattatcaattaataatataagatcaactattttacttgcaaatagattatatagtggaataaaagtaaaaatacaaagagtaaataattctagtactaatgataatttagtaagaaagaatgatcaagtatatataaattttgttgcatctaaaacacatttatttcctctttatgctgatactgctactacaaataaagaaaaaactataaagatttcatctagtggaaatagatttaatcaagtagttgtaatgaattctgtaggaaataattgtacaatgaattttaaaaataataatggaaataatattggattattaggatttaaagctgatactgttgttgcttcaacttggtattatacacatatgagagatcatactaatagtaatggttgtttttggaattttatatctgaagaacatggatggcaagaaaaataa

SEQ ID NO: 23 - DNA, 3759 bp, Artificial Sequence
  mat_peptide (1)...(3756): BoNT/E, C. crescentus-modified 1
atgccgaagatcaactccttcaactataacgacccggtcaatgaccgcaccatcctctacatcaagccgggcgggtgccaggagttctacaagagcttcaacatcatgaagaacatctggatcattccagaacgcaacgttatcggcacgaccccccaggacttccatcctccgacctcgctcaagaacggcgactccagttattacgacccgaactatctgcagagcgacgaggaaaaggaccgcttcctgaagatcgtgacgaagatcttcaatcgtatcaacaataacctcagcggcgggatcctcctggaggaactgagcaaggcgaacccgtatctgggaaatgataacacgccggacaaccagttccacatcggcgacgcctcggccgtcgagatcaaattctcgaacggttcgcaggacatcctgcttccgaacgtgatcattatgggcgcggagccggacctgttcgagaccaactcctcgaacatatcgctgcgcaacaattacatgccgtcgaaccacggcttcgggtccatcgcgatcgtcaccttcagcccggagtattcgttccgatttaacgacaacagcatgaacgagttcatccaggacccggccctgacgctgatgcacgaactcatccattccctgcacggcttgtacggcgcgaagggcatcaccacaaagtacacgatcacgcaaaagcagaacccgctcatcaccaacatccggggtaccaacatcgaagagtttctgaccttcggcgggaccgatctgaacatcattacgtcggcgcagtcgaacgatatctacaccaacctgcttgcggactataagaaaatcgcctcgaagctgagcaaggtccaggtgagcaaccccctgctaaatccctataaggacgtcttcgaggccaagtacggcctggataaagacgcctctggcatctactcggtcaacatcaacaagttcaacgatattttcaagaaactgtattcctttaccgagttcgacctggcgaccaagttccaggtcaagtgtcgccagacctatatcggccagtataagtacttcaagctgagcaacctgctcaatgactcgatctacaacatcagcgaggggtataacatcaacaatctgaaggtcaacttccgcggccagaacgcgaaccttaacccccgcatcattacgccgatcaccggccggggcctggtcaagaaaatcatacgcttctgcaagaatatcgtgtccgtcaagggcatccgcaagagcatctgcatcgagatcaacaatggcgagctgttctttgtcgccagcgagaactcgtataacgatgacaacatcaacacccccaaggagatcgatgacacggtgacctcgaacaataactacgaaaatgacctggaccaagtcatcttgaacttcaactcggagtcggcgcccggcctgtccgacgagaagctgaatctgacgatccagaacgacgcctacatccccaagtacgacagcaacggtacgtcggatatcgagcagcacgacgtcaacgagctgaatgtgttcttttacctggatgcccagaaggttcccgagggcgagaacaatgtgaacctgacgtcgagcatcgacaccgccctcctggagcagccgaagatctatacgttctttagtagcgagttcatcaacaatgtgaacaagccggtccaagccgcgctcttcgtctcttggatccagcaagtgctggtcgacttcaccacggaagcgaaccagaagtcgacggtggacaagatcgccgacatcagcatcgtcgtgccgtatatcggcctggcgctgaacatcggcaacgaggctcagaagggcaacttcaaggacgcgctggagctgctcggcgccggcatcctgctagagttcgagcctgagctgttgatccccaccatccttgtgttcaccattaagagcttcctcggctcgagcgacaataagaataaggtcattaaggccatcaacaatgccctgaaggagcgcgacgaaaagtggaaagaagtttactccttcatcgtgtcgaactggatgaccaagatcaacactcagttcaacaagcgcaaggaacagatgtaccaagccttgcagaatcaggtcaatgcgatcaagaccatcattgaatcgaagtataactcatacacgctcgaggaaaagaacgaactgaccaacaagtatgatatcaagcagatcgagaatgagctgaaccagaaggtctccatcgctatgaacaatatcgaccgcttccttactgagagctcgatctcgtacctgatgaagttgatcaatgaagtgaagatcaacaagctgcgggagtatgatgaaaacgtcaagacctacttgctgaactacatcatccagcacggttccatcctgggcgagagccagcaagagctgaactccatggtaaccgacaccctgaacaatagcatccccttcaagctgtcgagctacaccgatgacaagatcctgatcagctatttcaataagttctttaagcgcatcaagagctcctcggtgctgaacatgcggtacaagaacgataagtatgtcgatacctcgggctatgactccaacatcaacatcaatggcgacgtgtataagtaccccacgaacaagaaccagttcgggatctataacgacaagctgagcgaggtgaacatctcccagaacgactacatcatctacgacaacaagtacaagaacttcagcatcagcttctgggttcgcatcccgaactatgacaacaagatcgtgaatgtgaataacgagtataccatcatcaactgcatgcgcgacaacaatagtggctggaaggtctcactgaaccacaacgagatcatctggacactgcaggacaacgccggcatcaaccagaagctggccttcaactatggcaacgccaacggaatctcggactatatcaacaagtggatcttcgtgaccatcacgaacgaccgcctcggcgacagcaagctgtacatcaacggcaacctcatcgaccagaagtcgatcctgaacctgggcaacatccacgtctcggacaacatcctgttcaagatcgtgaattgctcgtacacccggtatatcggcatccggtatttcaacatcttcgacaaagaactggacgagaccgagatccagacgctgtacagcaacgaaccgaacacgaacatcttgaaggatttctggggaaactacctgctctatgacaaggagtactatctcctgaacgtcctgaagccaaacaatttcatcgatcgccggaaggattcgaccctcagcatcaacaatatccgctccaccatcctgcttgccaaccgtctgtactcaggcatcaaggtcaagatccagcgtgtgaacaactcgtccaccaacgacaacctggtgcgaaagaacgaccaggtctacatcaacttcgtggcatcgaagacgcacctgttccccctctacgccgacaccgccaccacgaacaaggagaagaccatcaagatctcgtctagcggcaacaggttcaaccaggtcgtggtcatgaactcggtcggcaacaactgcaccatgaacttcaagaacaacaacggcaacaacatcggtctgctgggtttcaaggccgacaccgtcgtcgcctcgacctggtactatacccatatgcgcgaccacaccaactcgaacggctgcttctggaactttatctcggaggaacatgggtggcaggagaagtaa

SEQ ID NO: 24 - DNA, 3759 bp, Artificial Sequence
  mat_peptide (1)...(3756): BoNT/E, C. crescentus-modified 2
atgccgaagatcaactcgttcaactacaacgacccggtcaacgaccgcaccatcctgtacatcaagccgggcggctgccaggagttctacaagtcgttcaacatcatgaagaacatctggatcatcccggagcgcaacgtcatcggcaccaccccgcaggacttccacccgccgacctcgctgaagaacggcgactcgtcgtactacgacccgaactacctgcagtcggacgaggagaaggaccgcttcctgaagatcgtcaccaagatcttcaaccgcatcaacaacaacctgtcgggcggcatcctgctggaggagctgtcgaaggccaacccgtacctgggcaacgacaacaccccggacaaccagttccacatcggcgacgcctcggccgtcgagatcaagttctcgaacggctcgcaggacatcctgctgccgaacgtcatcatcatgggcgccgagccggacctgttcgagaccaactcgtcgaacatctcgctgcgcaacaactacatgccgtcgaaccacggcttcggctcgatcgccatcgtcaccttctcgccggagtactcgttccgcttcaacgacaactcgatgaacgagttcatccaggacccggccctgaccctgatgcacgagctgatccactcgctgcacggcctgtacggcgccaagggcatcaccaccaagtacaccatcacccagaagcagaacccgctgatcaccaacatccgcggcaccaacatcgaggagttcctgaccttcggcggcaccgacctgaacatcatcacctcggcccagtcgaacgacatctacaccaacctgctggccgactacaagaagatcgcctcgaagctgtcgaaggtccaggtctcgaacccgctgctgaacccgtacaaggacgtcttcgaggccaagtacggcctggacaaggacgcctcgggcatctactcggtcaacatcaacaagttcaacgacatcttcaagaagctgtactcgttcaccgagttcgacctggccaccaagttccaggtcaagtgccgccagacctacatcggccagtacaagtacttcaagctgtcgaacctgctgaacgactcgatctacaacatctcggagggctacaacatcaacaacctgaaggtcaacttccgcggccagaacgccaacctgaacccgcgcatcatcaccccgatcaccggccgcggcctggtcaagaagatcatccgcttctgcaagaacatcgtctcggtcaagggcatccgcaagtcgatctgcatcgagatcaacaacggcgagctgttcttcgtcgcctcggagaactcgtacaacgacgacaacatcaacaccccgaaggagatcgacgacaccgtcacctcgaacaacaactacgagaacgacctggaccaggtcatcctgaacttcaactcggagtcggccccgggcctgtcggacgagaagctgaacctgaccatccagaacgacgcctacatcccgaagtacgactcgaacggcacctcggacatcgagcagcacgacgtcaacgagctgaacgtcttcttctacctggacgcccagaaggtcccggagggcgagaacaacgtcaacctgacctcgtcgatcgacaccgccctgctggagcagccgaagatctacaccttcttctcgtcggagttcatcaacaacgtcaacaagccggtccaggccgccctgttcgtctcgtggatccagcaggtcctggtcgacttcaccaccgaggccaaccagaagtcgaccgtcgacaagatcgccgacatctcgatcgtcgtcccgtacatcggcctggccctgaacatcggcaacgaggcccagaagggcaacttcaaggacgccctggagctgctgggcgccggcatcctgctggagttcgagccggagctgctgatcccgaccatcctggtcttcaccatcaagtcgttcctgggctcgtcggacaacaagaacaaggtcatcaaggccatcaacaacgccctgaaggagcgcgacgagaagtggaaggaggtctactcgttcatcgtctcgaactggatgaccaagatcaacacccagttcaacaagcgcaaggagcagatgtaccaggccctgcagaaccaggtcaacgccatcaagaccatcatcgagtcgaagtacaactcgtacaccctggaggagaagaacgagctgaccaacaagtacgacatcaagcagatcgagaacgagctgaaccagaaggtctcgatcgccatgaacaacatcgaccgcttcctgaccgagtcgtcgatctcgtacctgatgaagctgatcaacgaggtcaagatcaacaagctgcgcgagtacgacgagaacgtcaagacctacctgctgaactacatcatccagcacggctcgatcctgggcgagtcgcagcaggagctgaactcgatggtcaccgacaccctgaacaactcgatcccgttcaagctgtcgtcgtacaccgacgacaagatcctgatctcgtacttcaacaagttcttcaagcgcatcaagtcgtcgtcggtcctgaacatgcgctacaagaacgacaagtacgtcgacacctcgggctacgactcgaacatcaacatcaacggcgacgtctacaagtacccgaccaacaagaaccagttcggcatctacaacgacaagctgtcggaggtcaacatctcgcagaacgactacatcatctacgacaacaagtacaagaacttctcgatctcgttctgggtccgcatcccgaactacgacaacaagatcgtcaacgtcaacaacgagtacaccatcatcaactgcatgcgcgacaacaactcgggctggaaggtctcgctgaaccacaacgagatcatctggaccctgcaggacaacgccggcatcaaccagaagctggccttcaactacggcaacgccaacggcatctcggactacatcaacaagtggatcttcgtcaccatcaccaacgaccgcctgggcgactcgaagctgtacatcaacggcaacctgatcgaccagaagtcgatcctgaacctgggcaacatccacgtctcggacaacatcctgttcaagatcgtcaactgctcgtacacccgctacatcggcatccgctacttcaacatcttcgacaaggagctggacgagaccgagatccagaccctgtactcgaacgagccgaacaccaacatcctgaaggacttctggggcaactacctgctgtacgacaaggagtactacctgctgaacgtcctgaagccgaacaacttcatcgaccgccgcaaggactcgaccctgtcgatcaacaacatccgctcgaccatcctgctggccaaccgcctgtactcgggcatcaaggtcaagatccagcgcgtcaacaactcgtcgaccaacgacaacctggtccgcaagaacgaccaggtctacatcaacttcgtcgcctcgaagacccacctgttcccgctgtacgccgacaccgccaccaccaacaaggagaagaccatcaagatctcgtcgtcgggcaaccgcttcaaccaggtcgtcgtcatgaactcggtcggcaacaactgcaccatgaacttcaagaacaacaacggcaacaacatcggcctgctgggcttcaaggccgacaccgtcgtcgcctcgacctggtactacacccacatgcgcgaccacaccaactcgaacggctgcttctggaacttcatctcggaggagcacggctggcaggagaagtaa

SEQ ID NO: 25 - DNA, 3759 bp, Artificial Sequence
  mat_peptide (1)...(3756): BoNT/E, C. crescentus-modified 3
atgccgaagatcaacagcttcaactacaacgaccccgtcaacgaccggaccatcctgtatatcaagccgggcgggtgccaggagttctacaagtccttcaacatcatgaagaacatctggatcatccccgaacggaacgtgatcgggaccacgccgcaggacttccaccccccgacctcgctcaagaacggcgacagctcgtactatgacccgaactacctccagagcgacgaagagaaggaccgcttcctgaagatcgtcacgaagatcttcaaccgtatcaacaacaacctcagcgggggcatcctgctcgaggaactgtcgaaggccaacccgtatctggggaacgacaacacgcccgacaaccagttccatatcggcgacgcctccgccgtcgagatcaagttcagcaacggcagccaggacatcctgctccccaacgtcatcatcatgggcgccgaaccggacctgttcgagaccaactcgtccaacatcagcctgcgcaacaactacatgccgtccaaccacggcttcggctcgatcgccatcgtgaccttcagcccggagtactcgttccgcttcaacgacaactcgatgaacgagttcatccaggaccccgcgctgacgctgatgcacgagctgatccacagcctgcacggcctgtatggcgccaaggggatcacgaccaagtacaccatcacccagaagcagaaccccctgatcaccaacatccggggcaccaacatcgaggaattcctcaccttcggcgggaccgacctgaacatcatcacctcggcgcagagcaacgacatctacaccaacctgctcgcggactataagaagatcgcgtcgaagctgagcaaggtgcaggtgagcaacccgctgctcaacccgtacaaggacgtgttcgaggcgaagtacggcctggacaaggacgcctcgggcatctactcggtgaacatcaacaagttcaacgacatcttcaagaagctgtactcgttcaccgagttcgacctggccaccaagttccaggtgaagtgccggcagacctacatcggccagtacaagtacttcaagctgtccaacctgctcaacgacagcatctataacatcagcgagggctacaacatcaacaacctgaaggtcaacttccgcggccagaacgccaacctgaacccgcgcatcatcacgcccatcaccggccgcggcctcgtgaagaagatcatccgtttctgcaagaacatcgtctcggtgaagggcatccgcaagtcgatctgcatcgaaatcaacaacggcgagctgttcttcgtcgcgtcggagaactcgtataacgacgacaacatcaacacgccgaaggagatcgacgacacggtgacgagcaacaacaactacgagaacgacctggaccaggtcatcctgaacttcaactccgaatccgccccgggcctgtccgacgagaagctgaacctgaccatccagaacgacgcgtatatcccgaagtacgactcgaacggcaccagcgacatcgaacagcacgacgtcaacgagctcaacgtcttcttctacctggacgcccagaaggtcccggagggcgagaacaacgtcaacctgacctcgtccatcgacaccgccctgctcgagcagcccaagatctataccttcttcagctcggagttcatcaacaacgtgaacaagccggtccaggccgcgctgttcgtgtcgtggatccagcaggtgctggtggacttcacgaccgaagcgaaccagaagtcgaccgtcgacaagatcgccgacatcagcatcgtcgtgccgtatatcggcctggccctgaacatcggcaacgaggcccagaagggcaacttcaaggacgcgctggaactgctcggcgccggcatcctgctcgagttcgagcccgagctgctcatccccacgatcctggtcttcaccatcaagtccttcctgggctcgagcgacaacaagaacaaggtcatcaaggccatcaacaacgccctgaaggagcgcgacgagaagtggaaggaagtctactccttcatcgtctcgaactggatgaccaagatcaacacccagttcaacaagcgcaaggaacagatgtaccaggccctgcagaaccaggtcaacgcgatcaagacgatcatcgagtcgaagtacaactcgtatacgctggaggaaaagaacgagctgacgaacaagtatgacatcaagcagatcgagaacgagctgaaccagaaggtctccatcgccatgaacaacatcgaccggttcctgacggagtcgagcatctcgtatctcatgaagctgatcaacgaggtcaagatcaacaagctgcgcgagtacgacgagaacgtcaagacctacctgctcaactacatcatccagcacggctcgatcctgggcgagagccagcaggagctgaactcgatggtgacggacaccctgaacaactcgatcccgttcaagctgagctcgtataccgacgacaagatcctgatcagctacttcaacaagttcttcaagcgcatcaagtcgagctcggtgctgaacatgcgctataagaacgacaagtatgtcgacacgagcggctatgactccaacatcaacatcaacggcgacgtgtataagtacccgaccaacaagaaccagttcggcatctataacgacaagctgtccgaagtcaacatcagccagaacgactatatcatctatgacaacaagtacaagaacttctcgatcagcttctgggtccgcatcccgaactatgacaacaagatcgtgaacgtcaacaacgagtataccatcatcaactgcatgcgcgacaacaacagcggctggaaggtctcgctgaaccataacgaaatcatctggaccctgcaggacaacgccggcatcaaccagaagctggccttcaactacggcaacgccaacggcatctcggactatatcaacaagtggatcttcgtgaccatcacgaacgaccgtctgggcgactcgaagctgtatatcaacggcaacctgatcgaccagaagtcgatcctgaacctgggcaacatccatgtgtcggacaacatcctgttcaagatcgtcaactgctcgtacacgcgctatatcggcatccgctatttcaacatcttcgacaaggaactggacgaaaccgagatccagacgctgtattcgaacgagccgaacaccaacatcctgaaggacttctggggcaactatctgctctatgacaaggagtactatctgctgaacgtgctgaagccgaacaacttcatcgaccgccggaaggactcgaccctgtcgatcaacaacatccggagcaccatcctgctggcgaaccgcctgtactccggcatcaaggtcaagatccagcgcgtgaacaactcgagcaccaacgacaacctggtccgcaagaacgaccaggtctacatcaacttcgtcgccagcaagacgcatctgttccccctgtacgccgacaccgccaccacgaacaaggagaagaccatcaagatctcctcgtccggcaaccgcttcaaccaggtggtcgtgatgaactcggtcgggaacaactgcaccatgaacttcaagaacaacaacggcaacaacatcggcctgctgggcttcaaggcggacaccgtcgtcgcgtcgacctggtactacacccacatgcgcgaccacaccaactcgaacggctgcttctggaacttcatctcggaagagcacggctggcaggagaagtaa

SEQ ID NO: 26 - DNA, 3759 bp, Artificial Sequence
  mat_peptide (1)...(3756): BoNT/E, L. lactis-modified 1
atgccaaaaatcaatagcttcaattataatgatccggttaatgatagaacaatcttatatattaaaccaggaggttgtcaggaattttataaatcttttaatattatgaagaatatttggattatacctgaaagaaatgttattggtacaactccacaggattttcatccaccgacttcattaaaaaatggcgattcttcatattacgatcctaattatcttcaatcggatgaagagaaagacagatttcttaagattgttactaaaatttttaatcgaatcaataacaatctttcaggtggaatattattggaagagttatcgaaagctaacccctaccttggtaacgacaataccccagataaccaatttcacattggtgatgcctccgcggtcgaaattaaattttcaaatggctctcaagacattttacttcctaacgttattatcatgggagctgaacctgatctgtttgaaacaaacagtagcaacatcagtctgcgtaataactatatgccaagtaatcatggttttggctcgatcgctattgttacatttagtcctgaatattcattccgctttaatgacaatagtatgaatgagtttattcaagatcctgctttgactttaatgcatgaactaatacacagcctacatggtctttatggtgccaaaggcattactacgaagtatacaataacacaaaaacaaaatcccctaatcacaaatatacggggaactaacattgaagagttcctcactttcggcggtacagatcttaatattataacatcagcgcaaagtaacgatatatacacgaatcttttagcagactacaaaaagattgcatctaagcttagtaaagtccaagtatcaaatccattgttaaacccttataaagatgtttttgaggctaaatatggtttggataaagacgcgagtggtatttattcagttaatattaataaattcaatgacatttttaaaaagctatatagttttactgaatttgatcttgctacaaaattccaagtaaaatgtagacaaacctatatcgggcaatacaaatattttaaacttagtaatcttttaaatgattcgatttataatatttcagaagggtataatataaataacctaaaggttaactttagaggtcagaatgcgaatcttaatcctcgtataattactcctattactgggcgtggtttagttaaaaagattatccgtttttgtaaaaatatagtttccgtcaaaggtattaggaaatcaatttgtattgaaattaataacggagaattgttctttgtagcatcagaaaacagttataatgacgataacattaatacaccaaaagaaatagatgacaccgttacttcaaataacaattatgaaaatgacctagatcaagtaattttgaattttaattcagaatctgctccaggactctctgatgaaaaattaaatttaacaattcaaaacgatgcatatattccaaaatacgatagtaacggaacatcagatattgaacaacatgatgtaaatgaacttaatgtttttttctatctagatgctcaaaaagtgccagaaggagaaaataacgtcaatctcacaagctctattgatactgcattgttagaacaaccaaaaatttatacgttcttttcttcagaatttataaacaatgtgaataaacctgtacaagcagccttgtttgtatcatggattcaacaggttttagttgattttacaaccgaagcaaatcaaaaaagcactgtagataaaatcgctgatatttctattgtggttccttatattggactggctttaaatattggtaacgaagctcaaaaaggtaactttaaagatgccctcgaactgttaggtgcaggaatattattggaatttgagccagagttattgatccccacaattttagtgtttacaattaaatcattcttaggatcttcagataataaaaataaagtcattaaagcaattaataacgcacttaaggaacgtgacgaaaaatggaaagaagtatactcttttattgtttcgaattggatgacgaagataaatacacaatttaataaaagaaaagaacaaatgtatcaagccctacaaaatcaagtcaacgcaattaaaaccattatagagagtaaatacaacagttacactttggaagagaaaaatgaattgactaataaatacgatattaaacaaatcgaaaatgaattgaatcaaaaagtttcaattgctatgaataacatagatcgattcttgacggaatcttcaatttcttatttaatgaaacttataaatgaagtaaaaattaacaaattacgtgagtatgatgaaaatgttaagacatatttacttaattatatcattcaacacgggagtatcttaggagaatctcaacaggaactcaattcaatggttacagatacgctcaataactcaattcctttcaaattaagttcatatactgatgacaaaattctgatttcctatttcaataagtttttcaagagaatcaaatctagctctgttttgaatatgcgatacaaaaacgataaatatgttgacacaagcgggtatgattctaacatcaatattaatggagatgtctacaaatatccaactaataaaaaccaatttggaatttacaatgataaactttctgaagtaaatatcagtcaaaatgattatattatctatgataataaatacaaaaattttagtatttcattttgggttcgtattcctaattatgacaataaaattgtaaatgttaataacgagtatactattatcaattgtatgcgagataacaatagcggatggaaagtgtcccttaatcataatgaaattatctggactttgcaagacaacgctgggatcaatcaaaaattggctttcaattatgggaatgcaaatggaatttcagattacatcaataaatggatttttgtaactattacaaatgatcgtttgggtgattctaaattatatattaacggtaatttaatagaccaaaaatcaatcttaaatctcggaaatattcacgtatcagataatattctttttaaaatagttaactgctcttatacgcgatatattggtattcgttattttaatatttttgataaggaattggatgaaaccgaaattcaaactttatattctaatgagccaaatactaatattcttaaggacttttggggtaattacttattgtatgataaagaatattaccttttaaatgttttaaaaccgaacaattttatagatagacgcaaggacagtactctttccattaataacattagaagcactattttgttagccaatcgcctttatagtggcattaaagtcaaaatacaaagggttaataacagttcaaccaatgataatttagttcggaaaaatgaccaagtgtatatcaattttgttgctagtaagacgcatctttttccactatatgctgacacagcaactacaaataaagaaaagaccattaaaatttcttcatctggaaatcgtttcaatcaggtggtcgttatgaattctgttggtaataattgtacaatgaattttaaaaataataatggtaataatatcggcttgttaggatttaaggcagataccgtcgtggctagcacatggtattatacacatatgcgtgatcatacaaattctaatggatgcttttggaattttatctcagaagaacatggatggcaggaaaaataa

SEQ ID NO: 27 - DNA, 3759 bp, Artificial Sequence
  mat_peptide (1)...(3756): BoNT/E, L. lactis-modified 2
atgccaaaaattaattcatttaattataatgatccagttaatgatcgtacaattttatatattaaaccaggtggttgtcaagaattttataaatcatttaatattatgaaaaatatttggattattccagaacgtaatgttattggtacaacaccacaagattttcatccaccaacatcattaaaaaatggtgattcatcatattatgatccaaattatttacaatcagatgaagaaaaagatcgttttttaaaaattgttacaaaaatttttaatcgtattaataataatttatcaggtggtattttattagaagaattatcaaaagctaatccatatttaggtaatgataatacaccagataatcaatttcatattggtgatgcttcagctgttgaaattaaattttcaaatggttcacaagatattttattaccaaatgttattattatgggtgctgaaccagatttatttgaaacaaattcatcaaatatttcattacgtaataattatatgccatcaaatcatggttttggttcaattgctattgttacattttcaccagaatattcatttcgttttaatgataattcaatgaatgaatttattcaagatccagctttaacattaatgcatgaattaattcattcattacatggtttatatggtgctaaaggtattacaacaaaatatacaattacacaaaaacaaaatccattaattacaaatattcgtggtacaaatattgaagaatttttaacatttggtggtacagatttaaatattattacatcagctcaatcaaatgatatttatacaaatttattagctgattataaaaaaattgcttcaaaattatcaaaagttcaagtttcaaatccattattaaatccatataaagatgtttttgaagctaaatatggtttagataaagatgcttcaggtatttattcagttaatattaataaatttaatgatatttttaaaaaattatattcatttacagaatttgatttagctacaaaatttcaagttaaatgtcgtcaaacatatattggtcaatataaatattttaaattatcaaatttattaaatgattcaatttataatatttcagaaggttataatattaataatttaaaagttaattttcgtggtcaaaatgctaatttaaatccacgtattattacaccaattacaggtcgtggtttagttaaaaaaattattcgtttttgtaaaaatattgtttcagttaaaggtattcgtaaatcaatttgtattgaaattaataatggtgaattattttttgttgcttcagaaaattcatataatgatgataatattaatacaccaaaagaaattgatgatacagttacatcaaataataattatgaaaatgatttagatcaagttattttaaattttaattcagaatcagctccaggtttatcagatgaaaaattaaatttaacaattcaaaatgatgcttatattccaaaatatgattcaaatggtacatcagatattgaacaacatgatgttaatgaattaaatgtttttttttatttagatgctcaaaaagttccagaaggtgaaaataatgttaatttaacatcatcaattgatacagctttattagaacaaccaaaaatttatacatttttttcatcagaatttattaataatgttaataaaccagttcaagctgctttatttgtttcatggattcaacaagttttagttgattttacaacagaagctaatcaaaaatcaacagttgataaaattgctgatatttcaattgttgttccatatattggtttagctttaaatattggtaatgaagctcaaaaaggtaattttaaagatgctttagaattattaggtgctggtattttattagaatttgaaccagaattattaattccaacaattttagtttttacaattaaatcatttttaggttcatcagataataaaaataaagttattaaagctattaataatgctttaaaagaacgtgatgaaaaatggaaagaagtttattcatttattgtttcaaattggatgacaaaaattaatacacaatttaataaacgtaaagaacaaatgtatcaagctttacaaaatcaagttaatgctattaaaacaattattgaatcaaaatataattcatatacattagaagaaaaaaatgaattaacaaataaatatgatattaaacaaattgaaaatgaattaaatcaaaaagtttcaattgctatgaataatattgatcgttttttaacagaatcatcaatttcatatttaatgaaattaattaatgaagttaaaattaataaattacgtgaatatgatgaaaatgttaaaacatatttattaaattatattattcaacatggttcaattttaggtgaatcacaacaagaattaaattcaatggttacagatacattaaataattcaattccatttaaattatcatcatatacagatgataaaattttaatttcatattttaataaattttttaaacgtattaaatcatcatcagttttaaatatgcgttataaaaatgataaatatgttgatacatcaggttatgattcaaatattaatattaatggtgatgtttataaatatccaacaaataaaaatcaatttggtatttataatgataaattatcagaagttaatatttcacaaaatgattatattatttatgataataaatataaaaatttttcaatttcattttgggttcgtattccaaattatgataataaaattgttaatgttaataatgaatatacaattattaattgtatgcgtgataataattcaggttggaaagtttcattaaatcataatgaaattatttggacattacaagataatgctggtattaatcaaaaattagcttttaattatggtaatgctaatggtatttcagattatattaataaatggatttttgttacaattacaaatgatcgtttaggtgattcaaaattatatattaatggtaatttaattgatcaaaaatcaattttaaatttaggtaatattcatgtttcagataatattttatttaaaattgttaattgttcatatacacgttatattggtattcgttattttaatatttttgataaagaattagatgaaacagaaattcaaacattatattcaaatgaaccaaatacaaatattttaaaagatttttggggtaattatttattatatgataaagaatattatttattaaatgttttaaaaccaaataattttattgatcgtcgtaaagattcaacattatcaattaataatattcgttcaacaattttattagctaatcgtttatattcaggtattaaagttaaaattcaacgtgttaataattcatcaacaaatgataatttagttcgtaaaaatgatcaagtttatattaattttgttgcttcaaaaacacatttatttccattatatgctgatacagctacaacaaataaagaaaaaacaattaaaatttcatcatcaggtaatcgttttaatcaagttgttgttatgaattcagttggtaataattgtacaatgaattttaaaaataataatggtaataatattggtttattaggttttaaagctgatacagttgttgcttcaacatggtattatacacatatgcgtgatcatacaaattcaaatggttgtttttggaattttatttcagaagaacatggttggcaagaaaaataa

SEQ ID NO: 28 - DNA, 3759 bp, Artificial Sequence
  mat_peptide (1)...(3756): BoNT/E, L. lactis-modified 3
atgccaaaaattaattcatttaattacaatgaccctgtcaatgatcgtacaattctttatattaaaccaggaggttgtcaagaattttacaaatcttttaatatcatgaaaaatatttggattatccctgaacgaaatgttattggtactacaccacaagattttcatccacctacatcattaaaaaatggagatagttcatactatgatccaaattatcttcaatctgatgaagaaaaagatcgatttttaaaaatcgttactaaaatttttaatcgtattaataataatttatcaggtggaatcttactagaagaattatctaaagcaaatccttatttaggtaatgataataccccagataatcaatttcacattggtgacgcaagcgctgttgaaattaaattttctaatggttctcaagatattttacttccaaatgtgattatcatgggagccgaaccagatttatttgaaactaattcaagtaatatttctttacgtaataattacatgccaagcaatcatggatttggaagcattgctattgttactttttctccagaatactcttttcgctttaatgataatagtatgaatgaatttattcaagacccagccctaactctaatgcatgaattaatccatagtttacatggtttatatggagctaaaggtattacaaccaaatatacaattacacaaaaacaaaatcctcttatcacaaatattcgtggaacaaatattgaagaatttttaacctttggtggaaccgatttaaatattatcactagtgctcaatcaaatgacatttatacaaatcttttagccgattataaaaaaattgcttcaaaattgagtaaagtccaagtgtcaaatcctttattgaatccatataaagatgtatttgaagccaaatacggattagataaagatgcttcaggtatttacagtgtgaatattaataaatttaatgatatttttaaaaaactttattcatttactgaatttgatctagctactaaatttcaagtgaaatgtcgtcaaacttatattggtcaatataaatattttaaacttagtaatttacttaatgattctatttataatattagtgaaggttacaatattaataatttaaaagtgaattttagaggtcaaaatgctaatttaaatcctagaattatcacaccaattacaggtcgaggtttggtcaaaaaaattatccgtttttgtaaaaatattgtaagtgttaaaggtattcgtaaatcaatttgtattgaaattaataatggtgaacttttttttgtagcttcagaaaattcatataatgatgacaatattaatactcctaaagaaattgatgacacagtaacatcaaataataattatgaaaatgacttagatcaagttattcttaattttaattctgaaagtgcacctggtttgtcagatgaaaaattgaatttgactattcaaaatgatgcttacattccaaaatatgattctaatggaacatcagatattgaacaacatgatgtaaatgaactaaatgtttttttttatttagatgcacaaaaagttccagaaggagaaaataatgttaatttaacttctagtattgatactgctttgctagaacaaccaaaaatctacacatttttttcatctgaatttattaataatgtgaataaaccagtacaagcagccttatttgtctcatggatccaacaagttttggtagactttacaactgaagctaatcaaaaatctacagttgacaaaattgctgatatttctatcgttgtaccatacattggtttggctctaaatattggaaatgaagcacaaaaaggaaattttaaagatgcattagaattattgggtgcaggtatcttacttgaatttgaacctgaattacttattcctactatcttagtttttactatcaaatcatttcttggttcttcagataataaaaataaagtcattaaagccattaataatgcattaaaagaaagagatgaaaaatggaaagaagtttatagttttattgtttcaaattggatgacaaaaattaatacacaatttaataaacgcaaagaacaaatgtaccaagctttgcaaaatcaagttaatgcaatcaaaacaattatcgaaagtaaatacaattcttatacccttgaagaaaaaaatgaattaacaaataaatatgatattaaacaaattgaaaatgaattgaatcaaaaagtaagcattgcaatgaataatattgatagatttttaactgaaagttctatttcatatcttatgaaattgattaatgaagtaaaaattaataaattgcgtgaatatgatgaaaatgtgaaaacatatcttttaaattatatcattcaacacggaagcattttaggtgaatctcaacaagaattaaattcaatggtcactgatacactaaataattcaattccttttaaattaagctcatatactgatgacaaaattttgatctcatattttaataaattttttaaacgaattaaatctagttcagttttgaatatgagatataaaaatgacaaatatgtcgacaccagcggttacgactctaatattaatattaatggtgatgtgtacaaatatccaacaaataaaaatcaatttggtatttataatgataaattgagtgaagtcaatatctcacaaaatgattatatcatttatgataataaatataaaaatttttcaattagtttttgggtacgtattccaaattatgacaataaaattgttaatgtcaataatgaatatacaattatcaattgtatgcgagataataattcaggttggaaagtttcacttaatcacaatgaaattatctggacccttcaagacaatgctggaattaatcaaaaattagcatttaattatggaaatgctaatggtatttcagattatattaataaatggatttttgttacaattaccaatgatagattgggtgattctaaattatatattaatggtaatcttattgaccaaaaaagcattcttaatttgggaaatattcatgtttcagacaatattttatttaaaattgttaattgttcttatacacgttatattggtattagatattttaatatttttgataaagaattagatgaaacagaaattcaaacattatattcaaatgaacctaatacaaatattttaaaagacttttggggtaattatctattatatgataaagaatactatttgcttaatgttttaaaaccaaataattttattgatcgtagaaaagatagtaccttgtcaattaataatatccgtagtactattttgcttgctaatcgtttatactctggtattaaagttaaaattcaacgcgttaataattcaagtacaaatgacaatttagtacgtaaaaatgaccaagtttatatcaattttgtagcaagtaaaactcatttatttccactttatgctgatactgctacaactaataaagaaaaaacaattaaaatttcatctagcggtaatagatttaatcaagttgtagttatgaattcagtcggtaataattgtacaatgaattttaaaaataataatggtaataatattggtcttcttggatttaaagctgatacagttgttgcatcaacttggtattatacacatatgcgtgatcatactaatagcaatggatgtttttggaattttatttcagaagaacacggatggcaagaaaaataa

SEQ ID NO: 29 - DNA, 3759 bp, Artificial Sequence
  mat_peptide (1)...(3756): BoNT/E, M. extorquens-modified 1
atgccgaagatcaactccttcaactacaacgacccggtgaacgaccgcaccatcctgtacatcaagccgggcgggtgccaggagttctacaagtcgttcaatatcatgaagaacatctggatcattccggagcgcaacgtgatcggtacgaccccacaggacttccaccccccgacctccctcaagaacggcgacagctcctactatgacccgaactacctgcagtcggacgaggaaaaggaccggttcctgaagatagtcaccaagatctttaaccggatcaacaataacctctctggcgggatcctgctcgaggaactgagcaaggccaacccgtacctcggcaacgacaacaccccggataatcagttccacatcggcgatgcctcggccgtggagatcaagttctcgaacgggtcccaggatatcctgttgccgaacgtcatcattatgggtgcggagcccgacctgttcgagactaactcatcgaacatctcgctccgcaacaattacatgccgagtaaccatggcttcggcagcatcgccatcgtgaccttcagccccgagtacagcttccgattcaacgacaactcgatgaacgaattcatccaggatccggccctcacgctcatgcatgagctgatccacagcctgcacggcctctacggcgctaaggggatcaccacgaagtacacaatcacccagaagcagaacccgctgatcaccaacatccggggaaccaacatcgaggaattcctcaccttcggcggaaccgacctgaatatcattaccagcgcccagtcgaacgacatctacacgaacctcctggcggactacaagaaaatcgccagcaagctgtcgaaggtccaggtcagcaacccgctcctgaacccgtacaaggacgtcttcgaggcgaaatacggcctcgacaaggacgcgtcaggcatctacagcgtgaacatcaacaagtttaacgacatcttcaagaaactctacagcttcaccgagttcgacctggctaccaagttccaagttaagtgccgccagacctacattggccagtacaagtacttcaagctctccaatcttctcaacgactccatctacaacatcagcgagggctataacatcaacaatctgaaggtcaacttccggggccagaacgcgaacctgaacccgcgcatcattacgccgatcaccggccgcggcctcgtgaagaaaatcattcgcttctgcaagaatatcgtgtccgtgaagggcatccgcaagtcgatctgcatcgagatcaacaatggcgagctgtttttcgtcgcctcggagaactcgtacaacgacgataacatcaataccccgaaggagatcgacgataccgtcacctcgaacaataactacgagaacgatctggatcaggtcatcctgaacttcaactcggagagcgcaccgggcctgtcggatgagaagctgaaccttacgatccagaacgacgcctacatccccaagtacgacagcaacggcacctcggacatcgagcagcacgatgtgaacgaactgaacgtgttcttttacctcgacgcccagaaggtgcccgagggggagaacaatgtcaacctcacctcctctatcgacaccgcgctactggagcaaccgaagatctatacgttcttctcgtccgagttcatcaacaatgtcaacaagcccgtccaggcggccctgttcgtctcctggatccagcaagttctcgtggacttcaccacggaggcgaatcagaagtcgacggtcgacaagatcgccgatatctcgatcgtggtcccctacatcggtctcgcgctcaacatcggcaacgaggcccaaaagggcaacttcaaggatgccctcgaactgctcggcgccgggatcctgctcgagttcgagccggaactgctcatccccaccatcctcgtcttcaccatcaagtcgttcctcggcagctcggataataagaacaaggtgatcaaggcgatcaacaatgcgctcaaggaacgcgacgagaagtggaaggaggtctacagcttcatcgtgtcgaactggatgacgaagatcaacacccagttcaacaagcggaaggagcagatgtaccaggccttgcagaaccaggtgaacgccatcaagacgatcatcgagtccaaatataactcgtacacccttgaggaaaagaacgagctcaccaacaagtatgatatcaagcagatcgagaacgaactcaaccagaaggtgagcatcgccatgaacaatatcgaccggttcctgaccgagagctcgatctcgtacctcatgaagctcatcaacgaagtgaagatcaacaagctccgcgagtatgatgagaacgtcaagacctatctcctgaactatatcatccagcatggctcgatcctcggcgagtcgcagcaggaactgaacagcatggtcaccgacacactcaacaattccatcccgttcaagctctcgtcctataccgacgataagatcctgatcagctatttcaacaagttcttcaagcggatcaagtcgtcctcggtcctcaacatgcgctataagaacgacaagtacgtcgacacgtccggctacgacagcaatatcaacatcaacggcgatgtctacaagtaccctacgaacaagaaccagttcggcatctataacgataaactgtccgaggtgaacatctcgcagaacgactatatcatctatgacaacaagtacaaaaacttcagcatctccttctgggtccgcatccccaactacgacaacaagatcgtgaacgtgaacaatgagtataccatcatcaactgcatgcgcgacaacaattccggctggaaggtcagcctcaaccacaacgagatcatctggaccctccaggacaacgccggcatcaaccagaagctcgccttcaactacggcaacgccaacggcatctcggactacatcaacaagtggatcttcgtgacgatcaccaacgatcgcctcggtgactcgaagctctacatcaacgggaatctcatcgaccagaagagcatcctgaacctgggcaacatccacgtgtcggacaacatcctgttcaagatcgtgaactgctcctacacccgttacatcggcatccgctacttcaacatcttcgataaggagctcgatgagacggagatccagacgctctactccaacgaacccaacacgaacatcctgaaggacttctggggcaactacctgctctacgacaaggagtactatctgttgaacgtcctgaagcccaacaacttcatcgaccgtcggaaggactccacgttgtcgatcaacaacatccgctcgaccatcctgctcgcgaaccgcctttactcgggtatcaaggtgaagatccagcgcgtgaacaactcgtccaccaacgacaacctggtacgcaagaacgaccaggtgtacatcaacttcgttgcctccaagacgcatctgttccccctctacgcggacaccgccaccacgaacaaggagaagacgatcaagatctcgagctcgggcaacaggttcaaccaggtcgtggtcatgaactccgtcggcaacaactgcaccatgaacttcaagaacaacaacggcaacaacatcggcctgctgggcttcaaggcggacaccgtcgtcgcgtccacgtggtactacacgcacatgcgcgaccacaccaacagcaacggctgcttctggaacttcatctcggaggagcacggctggcaggagaagtaa

SEQ ID NO: 30 - DNA, 3759 bp, Artificial Sequence
  mat_peptide (1)...(3756): BoNT/E, M. extorquens-modified 2
atgccgaagatcaactcgttcaactacaacgacccggtcaacgaccgcaccatcctctacatcaagccgggcggctgccaggagttctacaagtcgttcaacatcatgaagaacatctggatcatcccggagcgcaacgtcatcggcaccaccccgcaggacttccacccgccgacctcgctcaagaacggcgactcgtcgtactacgacccgaactacctccagtcggacgaggagaaggaccgcttcctcaagatcgtcaccaagatcttcaaccgcatcaacaacaacctctcgggcggcatcctcctcgaggagctctcgaaggccaacccgtacctcggcaacgacaacaccccggacaaccagttccacatcggcgacgcctcggccgtcgagatcaagttctcgaacggctcgcaggacatcctcctcccgaacgtcatcatcatgggcgccgagccggacctcttcgagaccaactcgtcgaacatctcgctccgcaacaactacatgccgtcgaaccacggcttcggctcgatcgccatcgtcaccttctcgccggagtactcgttccgcttcaacgacaactcgatgaacgagttcatccaggacccggccctcaccctcatgcacgagctcatccactcgctccacggcctctacggcgccaagggcatcaccaccaagtacaccatcacccagaagcagaacccgctcatcaccaacatccgcggcaccaacatcgaggagttcctcaccttcggcggcaccgacctcaacatcatcacctcggcccagtcgaacgacatctacaccaacctcctcgccgactacaagaagatcgcctcgaagctctcgaaggtccaggtctcgaacccgctcctcaacccgtacaaggacgtcttcgaggccaagtacggcctcgacaaggacgcctcgggcatctactcggtcaacatcaacaagttcaacgacatcttcaagaagctctactcgttcaccgagttcgacctcgccaccaagttccaggtcaagtgccgccagacctacatcggccagtacaagtacttcaagctctcgaacctcctcaacgactcgatctacaacatctcggagggctacaacatcaacaacctcaaggtcaacttccgcggccagaacgccaacctcaacccgcgcatcatcaccccgatcaccggccgcggcctcgtcaagaagatcatccgcttctgcaagaacatcgtctcggtcaagggcatccgcaagtcgatctgcatcgagatcaacaacggcgagctcttcttcgtcgcctcggagaactcgtacaacgacgacaacatcaacaccccgaaggagatcgacgacaccgtcacctcgaacaacaactacgagaacgacctcgaccaggtcatcctcaacttcaactcggagtcggccccgggcctctcggacgagaagctcaacctcaccatccagaacgacgcctacatcccgaagtacgactcgaacggcacctcggacatcgagcagcacgacgtcaacgagctcaacgtcttcttctacctcgacgcccagaaggtcccggagggcgagaacaacgtcaacctcacctcgtcgatcgacaccgccctcctcgagcagccgaagatctacaccttcttctcgtcggagttcatcaacaacgtcaacaagccggtccaggccgccctcttcgtctcgtggatccagcaggtcctcgtcgacttcaccaccgaggccaaccagaagtcgaccgtcgacaagatcgccgacatctcgatcgtcgtcccgtacatcggcctcgccctcaacatcggcaacgaggcccagaagggcaacttcaaggacgccctcgagctcctcggcgccggcatcctcctcgagttcgagccggagctcctcatcccgaccatcctcgtcttcaccatcaagtcgttcctcggctcgtcggacaacaagaacaaggtcatcaaggccatcaacaacgccctcaaggagcgcgacgagaagtggaaggaggtctactcgttcatcgtctcgaactggatgaccaagatcaacacccagttcaacaagcgcaaggagcagatgtaccaggccctccagaaccaggtcaacgccatcaagaccatcatcgagtcgaagtacaactcgtacaccctcgaggagaagaacgagctcaccaacaagtacgacatcaagcagatcgagaacgagctcaaccagaaggtctcgatcgccatgaacaacatcgaccgcttcctcaccgagtcgtcgatctcgtacctcatgaagctcatcaacgaggtcaagatcaacaagctccgcgagtacgacgagaacgtcaagacctacctcctcaactacatcatccagcacggctcgatcctcggcgagtcgcagcaggagctcaactcgatggtcaccgacaccctcaacaactcgatcccgttcaagctctcgtcgtacaccgacgacaagatcctcatctcgtacttcaacaagttcttcaagcgcatcaagtcgtcgtcggtcctcaacatgcgctacaagaacgacaagtacgtcgacacctcgggctacgactcgaacatcaacatcaacggcgacgtctacaagtacccgaccaacaagaaccagttcggcatctacaacgacaagctctcggaggtcaacatctcgcagaacgactacatcatctacgacaacaagtacaagaacttctcgatctcgttctgggtccgcatcccgaactacgacaacaagatcgtcaacgtcaacaacgagtacaccatcatcaactgcatgcgcgacaacaactcgggctggaaggtctcgctcaaccacaacgagatcatctggaccctccaggacaacgccggcatcaaccagaagctcgccttcaactacggcaacgccaacggcatctcggactacatcaacaagtggatcttcgtcaccatcaccaacgaccgcctcggcgactcgaagctctacatcaacggcaacctcatcgaccagaagtcgatcctcaacctcggcaacatccacgtctcggacaacatcctcttcaagatcgtcaactgctcgtacacccgctacatcggcatccgctacttcaacatcttcgacaaggagctcgacgagaccgagatccagaccctctactcgaacgagccgaacaccaacatcctcaaggacttctggggcaactacctcctctacgacaaggagtactacctcctcaacgtcctcaagccgaacaacttcatcgaccgccgcaaggactcgaccctctcgatcaacaacatccgctcgaccatcctcctcgccaaccgcctctactcgggcatcaaggtcaagatccagcgcgtcaacaactcgtcgaccaacgacaacctcgtccgcaagaacgaccaggtctacatcaacttcgtcgcctcgaagacccacctcttcccgctctacgccgacaccgccaccaccaacaaggagaagaccatcaagatctcgtcgtcgggcaaccgcttcaaccaggtcgtcgtcatgaactcggtcggcaacaactgcaccatgaacttcaagaacaacaacggcaacaacatcggcctcctcggcttcaaggccgacaccgtcgtcgcctcgacctggtactacacccacatgcgcgaccacaccaactcgaacggctgcttctggaacttcatctcggaggagcacggctggcaggagaagtaa

SEQ ID NO: 31 - DNA, 3759 bp, Artificial Sequence
  mat_peptide (1)...(3756): BoNT/E, M. extorquens-modified 3
atgcccaagatcaactccttcaactacaacgacccggtcaacgatcggacgatcctctacatcaagccgggcggctgccaggagttctacaagtcgttcaacatcatgaagaacatctggatcatccccgagcgcaacgtcatcggcaccacgccccaggacttccatcccccgacgtccctcaagaacggcgactcgagctactacgacccgaactacctccagtccgacgaggagaaggatcgcttcctcaagatcgtgacgaagatcttcaaccgcatcaacaacaacctgagcggcggcatcctgctcgaggagctgtcgaaggccaacccctacctgggcaacgacaacacgccggacaaccagttccacatcggcgacgccagcgccgtcgagatcaagttctcgaacggctcccaggacatcctcctgccgaacgtgatcatcatgggcgccgagccggacctgttcgagacgaactcctcgaacatctccctccgcaacaactacatgccgtccaaccacggcttcggctcgatcgcgatcgtgaccttcagcccggagtactccttccgcttcaacgataactccatgaacgagttcatccaggacccggcgctcacgctcatgcatgagctcatccacagcctccatggcctctacggcgcgaagggcatcacgaccaagtacaccatcacccagaagcagaaccccctgatcaccaacatccgcggcaccaacatcgaggagttcctgacgttcggcggcacggatctcaacatcatcacgtcggcgcagagcaacgacatctacaccaacctgctcgccgactacaagaagatcgcctcgaagctctcgaaggtccaggtctcgaacccgctcctgaacccctacaaggacgtgttcgaggcgaagtacggcctcgataaggatgcgtccggcatctactccgtcaacatcaacaagttcaacgacatcttcaagaagctctacagcttcaccgagttcgacctcgccacgaagttccaggtgaagtgccgccagacctacatcggccagtacaagtacttcaagctctcgaacctgctcaacgactcgatctacaacatctcggagggctacaacatcaacaacctgaaggtcaacttccgcggccagaacgccaacctgaacccgcgcatcatcaccccgatcaccggccggggcctcgtgaagaagatcatccgcttctgcaagaacatcgtcagcgtgaagggcatccgcaagagcatctgcatcgagatcaacaacggcgagctgttcttcgtggcctcggagaactcctacaacgatgacaacatcaacacgccgaaggagatcgacgataccgtcacctccaacaacaactacgagaacgacctggatcaggtcatcctgaacttcaacagcgagagcgccccgggcctcagcgatgagaagctcaacctcaccatccagaacgatgcgtacatccccaagtacgattcgaacggcaccagcgatatcgagcagcacgacgtgaacgagctcaacgtcttcttctacctggacgcgcagaaggtcccggagggcgagaacaacgtcaacctcacctcgtccatcgacacggccctcctggagcagcccaagatctacaccttcttctcgagcgagttcatcaacaacgtgaacaagccggtccaggccgcgctgttcgtgtcctggatccagcaggtcctggtggacttcaccacggaggcgaaccagaagtccaccgtcgacaagatcgccgatatcagcatcgtcgtgccctacatcggcctggccctcaacatcggcaacgaggcgcagaagggcaacttcaaggacgcgctggagctgctcggcgccggcatcctcctggagttcgagccggagctgctcatcccgaccatcctcgtcttcaccatcaagtcgttcctcggctcgtccgacaacaagaacaaggtcatcaaggccatcaacaacgccctgaaggagcgggatgagaagtggaaggaggtctactcgttcatcgtgtcgaactggatgaccaagatcaacacccagttcaacaagcggaaggagcagatgtaccaggccctgcagaaccaggtgaacgccatcaagaccatcatcgagtcgaagtacaactcctacaccctcgaggagaagaacgagctgacgaacaagtacgacatcaagcagatcgagaacgagctcaaccagaaggtgtcgatcgccatgaacaacatcgatcgcttcctcaccgagagctcgatctcgtacctgatgaagctcatcaacgaggtcaagatcaacaagctgcgcgagtacgacgagaacgtgaagacgtacctcctgaactacatcatccagcatggctcgatcctgggcgagtcgcagcaggagctcaactcgatggtcaccgacaccctcaacaactccatccccttcaagctgtcgagctacaccgacgataagatcctcatctcgtacttcaacaagttcttcaagcgcatcaagagctcgtccgtcctcaacatgcgctacaagaacgacaagtacgtcgacacctccggctacgactcgaacatcaacatcaacggcgacgtgtacaagtacccgacgaacaagaaccagttcggcatctacaacgacaagctgtcggaggtgaacatcagccagaacgactacatcatctacgataacaagtacaagaacttctcgatctcgttctgggtccggatcccgaactacgacaacaagatcgtcaacgtcaacaacgagtacacgatcatcaactgcatgcgcgacaacaactccggctggaaggtgagcctgaaccacaacgagatcatctggaccctccaggacaacgcgggcatcaaccagaagctggccttcaactacggcaacgccaacggcatcagcgattacatcaacaagtggatcttcgtcacgatcaccaacgaccgcctgggcgactcgaagctgtacatcaacggcaacctcatcgatcagaagtcgatcctcaacctcggcaacatccacgtgtcggacaacatcctcttcaagatcgtgaactgcagctacacgcggtacatcggcatccggtacttcaacatcttcgacaaggagctggacgagaccgagatccagacgctgtactcgaacgagcccaacaccaacatcctcaaggatttctggggcaactacctcctgtacgacaaggagtactacctgctcaacgtgctgaagccgaacaacttcatcgaccgccggaaggactccaccctctcgatcaacaacatccgctcgaccatcctgctcgccaaccgcctctacagcggcatcaaggtgaagatccagcgcgtcaacaacagctccaccaacgacaacctcgtccgcaagaacgaccaggtctacatcaacttcgtcgcctccaagacccacctgttcccgctctacgcggacaccgccacgaccaacaaggagaagaccatcaagatctcgagctcgggcaaccgcttcaaccaggtggtcgtgatgaactcggtcggcaacaactgcaccatgaacttcaagaacaacaacggcaacaacatcggcctcctcggcttcaaggccgacaccgtggtggcgtcgacctggtactacacccacatgcgcgaccacaccaactcgaacggctgcttctggaacttcatcagcgaggagcacggctggcaggagaagtaa

SEQ ID NO: 32 - DNA, 3759 bp, Artificial Sequence
  mat_peptide (1)...(3756): BoNT/E, S. typhimurium-modified 1
atgccgaaaattaacagctttaattataatgacccggtaaacgatcgcaccattctctatattaaaccaggcggatgccaggagttttacaaatcttttaacatcatgaaaaatatctggataattcctgaaagaaatgtgatcggtaccactccccaagattttcatccgcctacctcactaaaaaacggagatagcagttattacgatccgaattacttacagagcgacgaggaaaaagaccgttttttgaaaatagtcaccaaaattttcaatcgcataaataacaatctgagcggcggtatccttctggaggaattgtccaaggccaatccgtacttaggtaacgataacacgcccgataatcagtttcatattggcgatgcctccgcagtagagattaagttcagcaatggaagccaagacatcctgctcccgaatgtgattatcatgggcgcagagccagatctgtttgagacaaacagcagtaacatttctctacgtaacaattacatgcctagtaaccacggctttggttcgattgcgattgtgacgttctcaccggagtattcatttcgtttcaatgataattcaatgaacgagtttattcaggatcccgcgctgaccctgatgcatgaacttattcattctctgcatggcctgtacggggcgaaaggcattaccacaaaatacaccattacgcagaaacaaaatcctttgatcaccaacattcgtggcacgaatatagaagagttcctgacgttcggtgggaccgacctgaatattatcaccagcgcgcaatcgaatgatatctatacgaatctactggccgattataaaaagatcgcgtctaaattaagcaaagtgcaggttagcaacccgctcctgaacccatataaagatgtcttcgaagcaaaatatggtttagataaagatgcctcgggcatttattcagtcaacattaacaaattcaacgacatcttcaagaaactgtactcgtttaccgaatttgatctggcgactaagttccaggtgaaatgccgtcaaacgtatatcgggcaatacaaatattttaagctgtccaatctactgaacgactccatatacaatattagcgaaggatataatattaacaatctgaaggttaatttccgcggccagaatgcaaatctgaacccgcgtattatcaccccgattacgggccgggggctcgtcaagaaaatcattcgcttctgcaagaacattgtttcagtcaagggcatccgcaaaagcatttgcattgaaattaataacggcgagctgttctttgttgcgagcgaaaactcgtataatgacgataatatcaacacgcccaaagagatcgatgacacggttaccagtaataacaattatgaaaacgatttagaccaagtgatcctgaactttaatagcgaaagcgctccgggcctgagcgacgaaaaactgaaccttactatccagaacgatgcgtatatcccgaagtatgactctaacggcacgtccgatatcgaacagcacgatgtaaacgaactgaacgtctttttctacttagatgcccaaaaagtgccggaaggtgaaaacaatgttaatcttacttcttcgatcgatacggcgctgttggagcaaccgaagatttacacattcttttcttcggagtttatcaacaatgtgaacaaaccagtacaggccgcgctgtttgtgtcctggattcaacaggtattagttgattttaccacggaagcgaaccagaagagcaccgtcgataaaatcgccgatatatcgatcgtcgtgccgtatattggcctggcgctgaatattggcaacgaagcccagaaaggcaatttcaaagacgcattagaactgcttggtgccggtatcttgctggaattcgaaccggaactcttaattcctacgatcctggtttttactattaaatcgtttctgggctccagcgataataaaaacaaagttatcaaagccattaataacgccttgaaagagcgcgacgaaaaatggaaagaagtgtactccttcattgtgtcgaactggatgaccaaaatcaacacgcagtttaacaaacgtaaagaacagatgtatcaggctttacagaatcaggtcaacgcaattaaaacaatcattgagtcgaaatacaattcctatacccttgaggaaaaaaacgagctgaccaacaaatatgatattaaacagatcgaaaatgagctgaaccagaaggtctcgatcgcgatgaacaatatcgatcgttttttgaccgaaagtagcatatcatacctgatgaaactgattaatgaagtaaaaataaacaaacttcgagagtacgatgaaaatgttaagacgtatttactgaattatattatccagcatggcagcatcctgggggaatctcagcaagagctgaactcgatggttaccgatactctgaacaatagcataccattcaaactgtccagttacaccgacgataaaatactgatctcatattttaacaaatttttcaagcggattaagtcgagctccgtgctgaatatgcgttacaaaaatgacaaatatgtcgacacttccggttacgacagcaatatcaacattaacggagatgtatacaaatacccgacgaacaagaatcagtttggcatttacaatgataagcttagcgaagtgaatatttctcagaatgattatattatctatgacaacaagtataaaaatttttctattagtttttgggttcgtattccgaactacgataataagattgttaacgtcaataacgaatatacaatcattaactgtatgcgggacaacaattcaggatggaaagtgagcttaaaccacaatgagatcatttggacgttgcaggataatgccggcatcaatcagaaactagcttttaattatggtaacgcgaacggcatctccgactatattaataaatggattttcgtgaccatcaccaacgatcgccttggtgatagcaagctgtatattaacggcaacctcattgaccagaaatcgatcttgaaccttgggaacattcacgtgagcgataacatcctctttaaaattgtcaattgctcttatacacgctacatcggcattcgctattttaatatttttgacaaagagttagatgaaacagaaatccagaccctctattcgaacgagccgaacacgaacatactgaaagatttttggggcaattatctgttgtacgacaaagaatactatctgctcaatgtgctgaaacccaataactttattgaccgtaggaaggattctaccctgtccattaacaatatccgcagcacgatccttctcgctaaccgcctgtactcaggcataaaggtaaaaatccagcgagtgaacaatagtagcacgaacgacaacttggtccgcaaaaatgaccaagtgtatatcaatttcgttgcgagtaaaactcatctgtttcctctgtatgcggacacggcgacgaccaacaaggagaaaaccattaaaatctcctcaagtgggaatcgcttcaaccaagtggtcgtaatgaacagtgtgggtaacaattgtaccatgaactttaaaaataacaatggaaataacatcgggttgctgggttttaaagcggataccgtcgtagcttccacctggtattatacgcacatgcgggaccatactaacagtaacggctgtttctggaacttcatttccgaagaacacgggtggcaggaaaaataa

SEQ ID NO: 33 - DNA, 3759 bp, Artificial Sequence
  mat_peptide (1)...(3756): BoNT/E, S. typhimurium-modified 2
atgccgaaaattaacagctttaactataacgatccggtgaacgatcgcaccattctgtatattaaaccgggcggctgccaggaattttataaaagctttaacattatgaaaaacatttggattattccggaacgcaacgtgattggcaccaccccgcaggattttcatccgccgaccagcctgaaaaacggcgatagcagctattatgatccgaactatctgcagagcgatgaagaaaaagatcgctttctgaaaattgtgaccaaaatttttaaccgcattaacaacaacctgagcggcggcattctgctggaagaactgagcaaagcgaacccgtatctgggcaacgataacaccccggataaccagtttcatattggcgatgcgagcgcggtggaaattaaatttagcaacggcagccaggatattctgctgccgaacgtgattattatgggcgcggaaccggatctgtttgaaaccaacagcagcaacattagcctgcgcaacaactatatgccgagcaaccatggctttggcagcattgcgattgtgacctttagcccggaatatagctttcgctttaacgataacagcatgaacgaatttattcaggatccggcgctgaccctgatgcatgaactgattcatagcctgcatggcctgtatggcgcgaaaggcattaccaccaaatataccattacccagaaacagaacccgctgattaccaacattcgcggcaccaacattgaagaatttctgacctttggcggcaccgatctgaacattattaccagcgcgcagagcaacgatatttataccaacctgctggcggattataaaaaaattgcgagcaaactgagcaaagtgcaggtgagcaacccgctgctgaacccgtataaagatgtgtttgaagcgaaatatggcctggataaagatgcgagcggcatttatagcgtgaacattaacaaatttaacgatatttttaaaaaactgtatagctttaccgaatttgatctggcgaccaaatttcaggtgaaatgccgccagacctatattggccagtataaatattttaaactgagcaacctgctgaacgatagcatttataacattagcgaaggctataacattaacaacctgaaagtgaactttcgcggccagaacgcgaacctgaacccgcgcattattaccccgattaccggccgcggcctggtgaaaaaaattattcgcttttgcaaaaacattgtgagcgtgaaaggcattcgcaaaagcatttgcattgaaattaacaacggcgaactgttttttgtggcgagcgaaaacagctataacgatgataacattaacaccccgaaagaaattgatgataccgtgaccagcaacaacaactatgaaaacgatctggatcaggtgattctgaactttaacagcgaaagcgcgccgggcctgagcgatgaaaaactgaacctgaccattcagaacgatgcgtatattccgaaatatgatagcaacggcaccagcgatattgaacagcatgatgtgaacgaactgaacgtgtttttttatctggatgcgcagaaagtgccggaaggcgaaaacaacgtgaacctgaccagcagcattgataccgcgctgctggaacagccgaaaatttataccttttttagcagcgaatttattaacaacgtgaacaaaccggtgcaggcggcgctgtttgtgagctggattcagcaggtgctggtggattttaccaccgaagcgaaccagaaaagcaccgtggataaaattgcggatattagcattgtggtgccgtatattggcctggcgctgaacattggcaacgaagcgcagaaaggcaactttaaagatgcgctggaactgctgggcgcgggcattctgctggaatttgaaccggaactgctgattccgaccattctggtgtttaccattaaaagctttctgggcagcagcgataacaaaaacaaagtgattaaagcgattaacaacgcgctgaaagaacgcgatgaaaaatggaaagaagtgtatagctttattgtgagcaactggatgaccaaaattaacacccagtttaacaaacgcaaagaacagatgtatcaggcgctgcagaaccaggtgaacgcgattaaaaccattattgaaagcaaatataacagctataccctggaagaaaaaaacgaactgaccaacaaatatgatattaaacagattgaaaacgaactgaaccagaaagtgagcattgcgatgaacaacattgatcgctttctgaccgaaagcagcattagctatctgatgaaactgattaacgaagtgaaaattaacaaactgcgcgaatatgatgaaaacgtgaaaacctatctgctgaactatattattcagcatggcagcattctgggcgaaagccagcaggaactgaacagcatggtgaccgataccctgaacaacagcattccgtttaaactgagcagctataccgatgataaaattctgattagctattttaacaaattttttaaacgcattaaaagcagcagcgtgctgaacatgcgctataaaaacgataaatatgtggataccagcggctatgatagcaacattaacattaacggcgatgtgtataaatatccgaccaacaaaaaccagtttggcatttataacgataaactgagcgaagtgaacattagccagaacgattatattatttatgataacaaatataaaaactttagcattagcttttgggtgcgcattccgaactatgataacaaaattgtgaacgtgaacaacgaatataccattattaactgcatgcgcgataacaacagcggctggaaagtgagcctgaaccataacgaaattatttggaccctgcaggataacgcgggcattaaccagaaactggcgtttaactatggcaacgcgaacggcattagcgattatattaacaaatggatttttgtgaccattaccaacgatcgcctgggcgatagcaaactgtatattaacggcaacctgattgatcagaaaagcattctgaacctgggcaacattcatgtgagcgataacattctgtttaaaattgtgaactgcagctatacccgctatattggcattcgctattttaacatttttgataaagaactggatgaaaccgaaattcagaccctgtatagcaacgaaccgaacaccaacattctgaaagatttttggggcaactatctgctgtatgataaagaatattatctgctgaacgtgctgaaaccgaacaactttattgatcgccgcaaagatagcaccctgagcattaacaacattcgcagcaccattctgctggcgaaccgcctgtatagcggcattaaagtgaaaattcagcgcgtgaacaacagcagcaccaacgataacctggtgcgcaaaaacgatcaggtgtatattaactttgtggcgagcaaaacccatctgtttccgctgtatgcggataccgcgaccaccaacaaagaaaaaaccattaaaattagcagcagcggcaaccgctttaaccaggtggtggtgatgaacagcgtgggcaacaactgcaccatgaactttaaaaacaacaacggcaacaacattggcctgctgggctttaaagcggataccgtggtggcgagcacctggtattatacccatatgcgcgatcataccaacagcaacggctgcttttggaactttattagcgaagaacatggctggcaggaaaaataa

SEQ ID NO: 34 - DNA, 3759 bp, Artificial Sequence
  mat_peptide (1)...(3756): BoNT/E, S. typhimurium-modified 3
atgccaaaaattaattccttcaattataatgacccggtaaacgatcgcacgattttgtacatcaaaccgggcggttgccaggaattttataaaagctttaatattatgaagaatatctggattatccctgagcgtaacgtcattggcacgacccctcaggacttccatccacctacctcgctgaaaaatggcgactcctcatattacgacccgaactacctgcagagcgatgaagagaaagatcgctttcttaaaattgtgacgaagatctttaaccgtattaataacaatctgagcggtggcattctgctcgaagagctgagcaaagcgaacccctacctgggcaatgacaacacccctgataaccagtttcacattggtgacgcgtctgcggttgaaatcaaatttagtaatggctcccaggatatcttgcttcctaatgttattatcatgggcgcagagccggatttatttgaaaccaacagttcaaacatttcgctgcggaataactacatgccgtcgaaccacgggttcggcagcattgcgattgtgaccttttctccggaatattccttccgtttcaacgacaactcaatgaatgaatttatccaggatccggcgctgacccttatgcacgaactgattcatagtctgcatggcctctatggcgcgaaaggcattaccacgaaatataccattacgcagaagcagaatccgctcattaccaatattcgcggcacgaatatcgaagagtttctgacgtttgggggtaccgacttgaatatcattacgagcgcgcaaagcaacgatatttataccaacctgttggcggattataaaaagattgctagcaagctgtcaaaggtacaggtatctaaccccttactgaacccgtacaaagatgtgtttgaagcaaagtatggccttgataaggatgcatcgggcatttacagcgtgaatattaacaaatttaacgacattttcaaaaagctgtatagcttcaccgagtttgatttagccacgaaattccaggttaaatgccgccagacctacattggtcagtataaatactttaaactgagcaatctcttgaatgattcaatctataatatctcggaaggctataacatcaacaatctgaaagtcaactttcgtggtcaaaatgctaatctcaatccgcgcatcattacccccatcaccggccgcggcctggtgaaaaagattatccgcttttgtaaaaacatcgtgtcggtaaaaggcatccgcaaatcaatctgcatcgagatcaacaatggcgagctgtttttcgtcgctagcgagaactcctacaatgatgacaacattaataccccgaaagagattgatgacaccgtcacgagcaataacaattacgaaaatgacctggaccaggtcattctgaacttcaatagtgaatcagcacccgggctgtcggatgaaaaacttaacctgaccattcagaacgatgcgtatattccaaagtatgacagtaacggcaccagcgacattgaacagcatgatgtcaatgaactcaatgtgttcttttacctggatgcacagaaagtgccggaaggggaaaacaatgtcaacctgacctccagtattgacaccgcgctgcttgaacagcccaaaatctatacctttttctcatcggaattcatcaacaatgtaaataagccagtacaagcggccctgtttgtttcctggatccaacaggtgcttgtagacttcacgaccgaagcgaaccagaaaagcaccgtcgataagatcgccgatatttcgatcgtcgtgccatacatcggcctggcgttgaacatcggcaatgaggctcaaaagggcaactttaaggatgctctggaattactgggcgccggtattctgctcgaatttgagccggagttattgatcccgaccatcttagtgtttacgattaaatcgttcttgggcagttccgataataaaaataaggtcatcaaagccattaataacgcgctgaaagaacgcgatgagaaatggaaagaggtgtactcttttatcgttagcaactggatgaccaaaatcaatacccagttcaataaacgcaaagaacagatgtatcaggccctgcaaaatcaggtcaacgccatcaaaaccattatcgaaagcaaatataatagctacacgctggaagagaaaaatgagctgaccaataaatacgatatcaagcaaattgagaatgagttaaaccagaaagtaagcattgccatgaacaatattgaccgttttctgaccgagagtagcatttcctatctcatgaaacttatcaacgaggtcaaaattaacaaactgcgcgaatatgatgaaaatgtgaagacgtacctgttaaactatattatccagcatggctctattctgggggaaagccaacaggaattaaactcgatggttaccgacaccctgaataacagcattcctttcaaactgtcttcatataccgatgacaagattctgatctcctattttaataaatttttcaagcgtatcaaaagttccagcgtgctgaatatgcggtataaaaatgataaatacgtggacaccagcggttatgactcgaatattaatattaacggtgatgtttataagtacccgacgaacaaaaatcagtttggcatttataacgataagctcagtgaagttaacatctcacagaatgattacatcatttatgataataaatataaaaacttttctatttccttctgggttcgtattccgaactacgataataaaattgtaaacgttaacaatgaatataccattatcaattgtatgcgtgataataactcgggctggaaagtgtcccttaaccacaacgaaatcatttggacgctgcaggacaacgcgggcattaaccaaaaattagcctttaactatgggaacgccaacgggatttctgattatatcaacaaatggatctttgtcacgattacgaacgaccgcttgggtgactccaaattatacattaacggtaatctgattgaccaaaaaagcatcttgaacttgggcaacattcacgtttctgataatatcctgtttaaaatcgtcaattgttcatatacccgttatattggcattcgttatttcaacattttcgataaagaactggacgagacggagattcaaacgctgtatagcaacgaaccgaacacgaacatccttaaagatttttggggtaactacttgttatatgataaagagtactatctgcttaacgtgctgaagccgaacaatttcatcgatcggcgcaaagatagtacgctgagcatcaacaatattcgttccaccattctgttagccaatcgcctctacagcggcattaaagtcaagattcagcgcgtgaataactcgtccaccaacgataacctggttcgcaaaaatgaccaagtgtatattaacttcgtggcgagcaaaacccatctgtttccgctgtacgcggataccgccaccacgaacaaagaaaaaaccattaaaatttcgtcttcgggcaaccggttcaatcaggtcgttgtgatgaacagtgttgggaataactgcaccatgaattttaaaaacaataacggtaataacattgggctgctgggttttaaagcggataccgtggtagcatctacctggtactatacgcatatgcgcgaccacaccaattctaacggctgcttttggaatttcatctccgaagaacatggctggcaagaaaagtaa

SEQ ID NO: 35 - DNA, 3759 bp, Artificial Sequence
  mat_peptide (1)...(3756): BoNT/E, P. pastoris-modified 1
atgccaaaaattaactccttcaattacaatgatcctgtaaacgatagaactattttgtatattaaaccaggcggttgtcaagagttttacaaatcatttaatatcatgaaaaacatttggataatcccagagcgcaatgtgataggtacgactccacaagatttccacccgcccactagcttaaaaaacggagactcttcatattacgatcctaactacctgcaaagtgacgaagagaaggatagattcctgaaaattgttacaaagattttcaacaggataaacaataacttgagtggcggtatccttttggaggaactttctaaagcaaatccttatctgggcaacgacaacaccccagacaaccaattccacatcggtgacgcctccgctgtagaaattaaattctctaatggatctcaggatatcttgctaccaaatgtgatcattatgggagcagagccagacctgttcgaaacaaactcttccaatatatcccttagaaataactacatgccttctaatcatggatttgggagcatcgcaatcgtgaccttttctcccgagtattcttttaggttcaatgacaactcaatgaacgaatttatccaggaccctgctctaacattgatgcacgaattgattcattctttacatggtttatatggagcaaagggaatcacaactaaatacacaattactcagaagcaaaatcctttgatcacaaatattagaggtacaaacatcgaagagtttttgacgtttgggggtacagatttaaatatcattacatccgcacaatctaacgatatttacacaaacctattggcggactataaaaagattgcttctaaactgtcgaaggtacaagttagtaatcccttgttaaacccttacaaggacgttttcgaggctaaatacggtttggacaaagatgcttcaggcatctatagcgttaatattaataaattcaacgatatctttaagaaattgtattccttcactgaatttgatttggccacgaagtttcaagttaaatgtcgacagacgtatattggccaatacaaatattttaagctcagcaacctcttgaacgactcaatatataatatttccgagggttataacattaacaatctgaaggttaacttcagaggacaaaatgctaacctaaacccaagaataattaccccaatcactggtcgtggtttggttaaaaagattatccgtttttgcaaaaatattgtgtctgttaagggtattcgaaagtcgatctgtattgaaatcaacaatggtgaattgtttttcgtagcctccgaaaactcctacaacgatgacaacattaacactccgaaggagattgatgacactgtcacgtctaacaataactacgagaatgacttagaccaagttatactaaatttcaactctgagagcgcccctgggttatctgatgaaaaattgaaccttactattcaaaacgacgcttatattcctaaatatgattctaatggaacgtctgatatcgaacaacatgacgtcaatgaattgaacgtctttttctatctagacgcacaaaaggtcccagagggagagaataacgtcaatttaacaagttcgattgataccgctttgctagaacagccaaagatctacacctttttctctagtgaattcataaacaatgttaacaagccagttcaagccgctttatttgtgtcgtggattcagcaagtccttgttgatttcactaccgaagccaatcagaagtcaaccgttgataagatagccgatatttctattgtcgtaccttacattgggttggctctgaatattggaaacgaagcacaaaaaggtaactttaaagacgcattagaactcctgggtgctggaatcttgctggagttcgagccagagctgttgattcccacaatcttggtgttcacaattaagtcctttctaggatcttcagataataaaaacaaagtgatcaaggcaattaataacgctttgaaagaaagggacgaaaaatggaaggaagtttacagctttatcgtcagtaactggatgaccaagattaacacccaattcaataagagaaaggaacagatgtaccaggctttgcaaaatcaggtgaacgctataaagactattatcgagtctaaatacaactcttacacactggaggaaaagaatgagctgactaacaaatatgacattaaacaaattgaaaacgaactcaatcagaaggttagtatcgctatgaataacatagatagattcttgaccgagtctagtatttcttacttaatgaaattgataaatgaggttaagataaacaaattaagagaatacgatgaaaacgttaagacttacttacttaattacattatacaacacggttctatacttggtgagtctcaacaggagctgaattctatggttactgacacccttaacaattcaataccctttaagcttagttcctatactgatgacaagatactaattagttacttcaataagtttttcaagagaattaaatcatcctcagttcttaacatgcgatacaaaaacgataaatatgttgatactagtggttacgattccaacataaacatcaatggggatgtttataagtatccgactaacaagaaccagtttggaatttataatgataagctatcagaggttaatatctcacaaaatgattatattatctacgacaataagtataagaatttcagtatttcattctgggtccgcatccctaactacgacaacaagattgttaacgtaaacaatgagtacactattataaactgtatgagagataacaattccggttggaaggtctcgctgaatcacaacgaaattatatggacgcttcaggataatgctggtatcaaccaaaagttagcatttaattatggtaatgccaacggaatttcagattacattaataagtggatctttgttactattaccaatgatagacttggcgatagtaaattgtatattaacggaaacctaattgatcaaaaaagcattctgaatctcggtaatatccatgtctccgacaatattttgttcaagattgttaactgctcatatactaggtacatcggtattcggtattttaatatatttgataaggaacttgacgaaacagaaatccagaccctttatagtaacgagcctaatacgaacattttaaaagacttttgggggaactacttgctgtacgataaggagtactatctcttgaacgtcctaaagccaaacaattttatcgacagacgtaaagactctactttgtcaataaacaatatacggagtaccatcctccttgctaaccgtttgtactcaggaattaaagtgaaaattcaaagggtaaataactcgtccacaaacgataatctcgttcgtaagaatgatcaggtctacattaactttgtcgcgtccaaaactcatttgttccccctttatgctgataccgccacaactaataaagaaaagactatcaaaattagctcatcgggtaatagatttaatcaagtcgtagtgatgaattcggtgggcaataactgtaccatgaattttaagaacaataacggcaacaatatcggtttacttggatttaaggccgatactgtagtggcctccacttggtactacacccatatgagagatcatactaattccaacggatgcttctggaactttatcagcgaagaacacggttggcaggaaaagtaa

SEQ ID NO: 36 - DNA, 3759 bp, Artificial Sequence
  mat_peptide (1)...(3756): BoNT/E, P. pastoris-modified 2
atgccaaagatcaactcgttcaactataacgaccctgttaatgatcgtaccatcctatatattaagcctggtgggtgtcaggaattttataagtcattcaatattatgaagaatatttggattataccggagagaaatgtcattgggaccactccccaagactttcatcctcccactagtctaaaaaatggtgactcatcctactatgaccctaattacctccaatccgatgaagagaaggatagatttctgaagattgtcaccaaaatctttaacagaattaataacaatttgtctggtggcattctgttggaagagctgagtaaagccaacccgtacctcggtaatgacaatacgccagataaccaatttcacataggtgacgcatcagcggtagagattaaatttagcaacggttcacaggatatcctgttgcctaatgttataattatgggagctgaaccagatcttttcgaaactaactcatccaatatctccttaaggaacaattatatgccatcgaatcacggatttggctcgattgctattgttacattcagcccagagtactcattcaggttcaacgacaactccatgaacgaatttatccaagatccagcattgacgctgatgcatgaacttattcatagcttgcacggcctttacggagccaagggtattacaactaaatataccattactcaaaagcagaaccctttgattactaacatccgtggaactaacatagaggaattcctaaccttcggtggaacggaccttaatataatcacctccgctcaatcaaacgatatttatacaaatttgctagcagattataagaaaattgcctccaaattgagcaaagtacaagtctcaaaccctttgcttaacccatataaagacgttttcgaggctaagtacggtctagataaagacgccagcggtatttattcggttaatattaataagtttaatgatatatttaaaaagttatacagctttacagagtttgatctggcaaccaaattccaggtgaagtgtagacaaacctacatcggtcagtataagtacttcaaactgtcaaacctcttgaacgactcaatctataatatttctgaaggatataacataaataacttgaaagttaacttccgaggacagaacgctaatttgaatcctagaattatcacacctatcaccggccggggactggtgaaaaagattatcagattttgcaagaacatcgtttccgttaaaggaataagaaaaagtatttgcatcgaaatcaataacggcgaactcttctttgttgcttctgaaaactcatacaacgacgataatatcaatacgcccaaagagattgacgatactgttaccagtaataacaattacgagaatgacctggatcaagtcatcctaaattttaacagtgagtctgctccagggttgtcagacgaaaagcttaacttgacgatacagaatgatgcttatattcctaaatacgattccaatggtacttctgatattgaacaacatgacgttaacgaattgaacgttttcttttatttggacgcccaaaaggttcccgaaggagaaaacaatgtgaacttgacatcctctattgatacagcccttttggaacaaccaaaaatttacacatttttctcgtctgaattcatcaataacgtcaacaaacctgtgcaagcggctttatttgtgtcttggatacagcaagttctggtagatttcacaactgaggctaaccaaaagagtactgttgataagatagctgatatctccatcgttgtcccctacattggtctagctttgaacattggtaacgaagctcagaaaggtaactttaaggatgctttagaattacttggtgcaggaattctcttggagttcgagccagaactacttattccgacgatcttagtgttcacaattaagagtttccttgggtcatctgataataaaaacaaggttattaaggccattaacaatgctttaaaggaaagagatgaaaaatggaaggaggtttactcttttatcgtgtcaaattggatgactaaaattaatactcagtttaataagcggaaggaacaaatgtaccaggcattacaaaaccaagtcaatgccattaaaactataatcgagtccaagtacaattcttatacacttgaggaaaaaaacgaacttaccaataaatacgatattaaacaaatcgagaacgagttgaatcaaaaagtctctatagcaatgaataacattgacaggttcttgactgaatcttccatctcttatctgatgaaattgattaacgaagtcaaaattaacaagttgcgtgagtacgatgagaatgttaagacatatcttttgaattatataattcaacatggtagcattttaggtgaatctcagcaagagttaaactccatggtaactgacacgttgaataacagcataccttttaaattgagttcttatactgacgataagatcctgatttcgtatttcaataagttctttaaacgcatcaagtctagttctgtccttaatatgaggtacaagaacgacaaatacgtcgatacttctggatatgattctaatattaacattaatggcgatgtctataagtacccaaccaataagaatcaatttggtatctacaatgacaaactttccgaagttaatatatctcaaaatgactacattatatacgacaacaagtataaaaactttagtataagtttttgggttagaatccccaactatgacaacaagattgtcaacgtaaataacgagtacactattatcaactgtatgagagataataactctggctggaaggtttcgctcaaccataacgaaattatatggacactgcaggataatgcaggaattaaccagaagcttgccttcaattacggtaacgccaacggaatctccgattacatcaacaagtggatttttgtgactattaccaatgatagactgggggactcgaaactctatattaacggtaaccttatagaccagaagtctatcctaaatttgggtaacatccatgtttcagataatattctatttaagatcgttaactgtagttacactagatatattggtatcagatattttaacatatttgacaaggaattggatgaaactgagattcaaaccttgtacagcaacgaaccaaacactaacatactcaaggatttttggggaaactacttactatatgataaggagtactatttattgaacgtcttaaagccaaacaattttattgatagaaggaaggactctactttatccattaataacattcgatctaccattctgttagccaaccgcttgtactccggtatcaaggtgaaaatccaaagagtaaacaattctagtacaaacgacaatttggttcgtaaaaatgatcaagtatacatcaacttcgtggcatcaaagactcacttattcccactatacgctgatactgcaaccacaaacaaggagaaaaccataaaaattagttcaagtgggaatcgttttaaccaggtggtagttatgaattctgtcggaaataactgtacaatgaatttcaagaataacaatggtaataacatcggactgttgggcttcaaagctgatacagtggtagcttctacttggtactacactcacatgcgagaccacacgaattccaatggttgcttctggaattttatttcagaggaacatggatggcaggagaaataa

SEQ ID NO: 37 - DNA, 3759 bp, Artificial Sequence
  mat_peptide (1)...(3756): BoNT/E, P. pastoris-modified 3
atgccaaagatcaatagtttcaactacaacgatccggttaatgacagaactattctgtacattaagcctggtggctgtcaggagttctataaaagctttaacattatgaagaatatttggattatacccgagcgaaatgtgattggaacaactccacaagatttccatccgccaacatccttgaaaaatggtgattcttcctactatgatccaaactacctgcaatcagacgaagagaaggaccggtttctaaagatcgttaccaagatatttaatagaatcaataacaatctttcaggcggtatcttgttagaggaactgtctaaagccaatccctacttgggtaatgacaacacaccagacaatcagttccatatcggtgatgcttccgctgttgaaattaagttctccaacgggtcccaagatattcttctgcccaatgtgattatcatgggagctgaaccagacctttttgaaactaactcgtctaacatatctctacgtaacaattacatgccaagtaatcatggttttggaagtatcgcaatcgttacttttagtcctgagtatagttttagatttaatgataactctatgaacgagtttatccaagacccagctttgacgttgatgcacgaattaattcactctttacacggattgtatggcgctaaaggaatcacaactaaatacacaattactcaaaaacagaaccctttgataacgaatatacgtggcactaacattgaggaatttttaacatttggtggaactgatcttaatatcattacctctgcccaatccaacgacatatacactaatttgctcgcagattacaagaaaatcgcctctaagctttctaaagtgcaggtatcaaatcctttgctaaacccttataaggatgtatttgaggctaagtatggtttggacaaagacgccagcggtatttattccgtgaatataaacaagtttaatgatattttcaaaaagttatactccttcacagagttcgatctagcaacaaagtttcaggtcaagtgtagacaaacttatatcggacagtataagtacttcaaactatcaaacttacttaatgattccatatataacatttcagagggttacaacattaacaatcttaaggtaaacttcagaggacaaaatgcaaacttaaaccctagaattatcactcctattaccggcagagggctggttaagaaaatcattcgcttctgcaaaaacattgtatcggttaagggtattaggaaaagtatttgcatcgaaatcaacaatggtgaattgttctttgtggcttctgagaactcatacaacgatgacaacattaatactcctaaggaaatcgatgacactgtcacctccaataacaattatgagaatgacctcgatcaagtgatattaaactttaattcagaaagcgctccaggattatcagacgagaagttaaatcttactatacagaacgatgcttacatacccaaatacgacagtaacggtacttcagacattgagcaacatgatgttaatgaactgaacgtctttttctatttagacgcccaaaaggttcctgagggtgaaaataacgtcaatttgacgagctcaatcgatactgctttactggaacaaccaaagatttacacctttttctcttccgaattcattaataacgtcaataaacccgttcaagcagctctattcgtttcatggattcaacaggttttggtcgatttcactacagaggccaatcaaaaatcaactgtcgataagatagccgacatttcgattgtggtaccatacattggtttggccttaaacatcggtaatgaggctcaaaagggaaacttcaaagatgcacttgaacttttgggtgcgggaattcttttagaatttgagcctgagctgttgattccaaccattcttgtattcactattaaatcgtttctgggatctagcgataataaaaacaaggtcatcaaggcaattaataacgcattaaaagaaagagatgaaaagtggaaagaggtttacagctttatcgtgtcgaattggatgacaaaaatcaacacacagttcaacaagagaaaagaacaaatgtatcaagccctacaaaatcaagtcaatgccattaaaacaattatcgaatcgaagtacaactcttacactctagaggaaaaaaacgaactgaccaacaaatatgacattaagcagatcgaaaatgaattgaaccaaaaagtctctatcgcaatgaataacattgatagatttctgactgaatcttcgatatcttaccttatgaaacttataaacgaggttaagattaataaactaagagaatatgatgaaaatgtcaagacatatttgctgaactacataattcaacacggttcaatcctcggggaatctcagcaagaactaaactctatggtcacggataccctcaacaatagtataccctttaagttgtccagctacaccgacgataagattctaataagctattttaataagttctttaagagaatcaagtcctcatctgttttgaacatgaggtacaagaacgataaatacgtagacacttcgggatacgactcaaacattaatatcaacggcgatgtttataagtaccctacaaataagaaccagttcggtatctataatgataagttgtcagaagtgaatatctctcagaacgactatattatctacgataacaagtataaaaattttagtatcagtttctgggtgcgaataccaaactacgataacaagatagtcaacgtcaacaatgagtacaccattatcaactgtatgagagacaacaattcaggatggaaggtgagtttgaatcacaacgagattatctggacgctgcaggataacgcaggtatcaatcaaaaactcgcttttaactacggtaatgctaacggtatttccgattatattaataaatggatatttgttacgattactaacgataggctaggagattctaaattgtacattaatggtaacttgatcgaccaaaaatccattctgaacttgggaaacattcatgtttccgataatatcttgttcaagattgttaactgttcctataccagatatattggtataaggtacttcaacattttcgacaaagaattggacgagactgaaatacagaccctttattccaacgaaccgaacaccaacatattgaaggatttttggggtaattatttgctgtatgataaggagtattaccttttgaatgttttgaaacctaacaattttattgaccgacggaaggattctactttgtctattaacaatattagaagcactattttactggcgaaccgtttgtattctggaattaaagtcaagattcagagggtgaataactcttccacaaatgataatcttgtacgcaagaacgaccaagtttacatcaacttcgttgccagtaaaacacatttgttcccactctacgctgatactgctacgacaaataaagaaaaaaccatcaaaatttctagttctggtaaccgttttaatcaagttgtagttatgaactcagttggcaataactgtactatgaatttcaaaaataacaatggaaataacattgggctcctcgggtttaaggctgacaccgttgttgctagtacgtggtattacacccacatgcgtgaccataccaactctaatggctgcttttggaattttatcagtgaagagcatgggtggcaggagaagtaa

SEQ ID NO: 38 - DNA, 3759 bp, Artificial Sequence
  mat_peptide (1)...(3756): BoNT/E, S. cerevisiae-modified 1
atgccaaaaattaattcatttaactacaatgaccctgtaaatgacagaacaatcttgtacattaaacctggtgggtgtcaagagttttacaaaagtttcaacatcatgaagaatatttggattataccggaaaggaatgttataggtactacaccacaagattttcatccacctacttccctaaaaaacggggattcatcgtattacgaccctaactatttacaaagcgacgaggaaaaggataggttccttaagatcgttacaaaaatctttaatagaatcaacaataacttgagcggaggtatcttgttagaagagcttagtaaagcaaatccatacttaggtaacgataatactccagataaccaatttcatattggtgatgcatccgctgttgaaattaagttctctaatggttcacaagatatactcttaccaaatgttattatcatgggcgctgaaccagatctattcgaaaccaactcctctaacatctcactgagaaataactacatgccgtcgaatcacggctttggttctattgcgatcgtgacgttttcacccgaatactcgttcagatttaacgataatagcatgaatgaatttattcaagatcctgctttaactttaatgcacgagttaatacatagtttgcatggcttgtacggagctaaaggcattaccacaaagtatactataactcaaaagcaaaatccgttgattaccaatataagaggtaccaatatagaggaattcttaacatttggtggaactgatttgaatattataacttcagcccagtctaacgatatttatactaatttactggctgattacaaaaagatcgcttctaaattgagtaaggtgcaagtctctaacccactattgaacccctacaaagacgtttttgaggccaaatacggattggacaaggatgcaagtggcatctactctgttaatattaacaagttcaatgatatttttaagaaactatactcctttacagaatttgatttggcaacaaaatttcaagtaaaatgtagacaaacgtatatcggtcaatataaatacttcaaattatctaacttactaaacgattctatctacaatatcagcgaaggatataatataaacaatttaaaagtaaatttccgaggacagaatgcaaacttaaacccaagaatcataactcccattacgggtagaggtctagtcaaaaagatcattcgattctgtaagaatattgtgagcgttaaaggtatacggaagagtatctgcatagaaattaataacggcgaattatttttcgttgcttctgagaattcatacaacgacgataacattaatactccaaaagaaattgatgacacagtcacaagcaacaataactatgaaaatgacttagaccaagtaatcctaaatttcaattcggaatcagctccaggtttatccgatgaaaaactgaacctaacaatacagaatgatgcgtacataccaaaatacgattctaatggcacatcagacattgagcagcatgacgtcaatgagctaaatgtatttttctatttggatgctcaaaaggtcccagaaggtgaaaataacgttaacctaaccagttcaatagacaccgcgcttttagaacaacctaaaatctatactttctttagttctgagtttatcaataacgtcaataagcctgtccaagccgcactgtttgttagttggatccaacaggtgttggttgattttaccacggaagcaaaccagaagtcgacagttgacaaaattgccgatatatcaatagtagttccctatattggattagctctcaatataggaaatgaagctcaaaagggtaattttaaagacgctttggaacttttgggcgctggtatattacttgaatttgaaccagagttgctgattccgactatcctggtctttaccataaaatcttttttaggatctagtgataacaaaaataaagtaattaaggcaattaataacgcactaaaagaaagggatgaaaaatggaaggaagtgtattcattcatcgtgtccaattggatgactaaaataaatacccaattcaacaagcgcaaggaacaaatgtatcaggccttgcagaaccaagtgaatgcgataaaaacaattatcgagtctaaatataattcgtacactttggaggaaaaaaatgaactgactaataaatacgatatcaagcagattgaaaatgaattaaaccaaaaagtgagtatagccatgaataacatcgataggtttttgaccgaatcttccatttcctatttgatgaaattgattaatgaggttaaaattaataaattgagagaatatgatgagaacgttaaaacgtatctattaaactacattatacaacatggctccatcttgggtgaatctcaacaggagctgaatagcatggtcacagatacactgaacaattcaatacccttcaagttgtcgtcatacacggacgataagatccttatttcctacttcaacaagtttttcaagagaatcaaaagtagctcagtcttaaatatgcgctataaaaatgataagtatgtagacacttctggatatgactctaatattaacatcaatggtgacgtgtataagtaccctacgaacaagaaccagttcggcatttataacgacaagttaagcgaagttaatataagtcaaaatgactatattatatacgacaacaaatataaaaatttttcgatatctttctgggttaggattcctaactatgataataagatcgtgaatgtaaataacgaatatacaattataaactgtatgcgtgataacaattcgggttggaaggtgagtctaaaccataacgaaattatatggacactccaggataacgcagggattaatcaaaaattggcatttaattacgggaatgccaacggcatttctgattatattaataagtggattttcgtaacaattactaacgatagactgggtgattcaaaattatatattaatgggaatctcattgaccaaaaaagtattttgaatcttggtaatatccacgtaagcgacaatatcctttttaagatagttaattgctcttataccagatatattggtattcgttacttcaacatttttgataaggagttggacgagaccgaaattcaaacgctctactcaaatgaacctaatacgaacattctgaaggatttttggggtaattatttgctttatgataaagaatactatttgttaaacgttctcaaaccaaacaatttcatagatagaaggaaagactccactctatctataaataacattcgttctaccattttgcttgccaatcgtctttattcaggaattaaagttaaaattcaaagggttaataactcctctacaaatgataaccttgtcagaaagaatgatcaggtttatattaattttgtggcatcaaaaactcaccttttccctttatatgccgatactgctactaccaataaagagaagacgataaagatttcctcaagcgggaacagatttaaccaagtcgttgtaatgaattccgttggtaataactgtactatgaattttaagaacaataacggaaataacatcggtctattagggttcaaagcggatacagtagtcgcttctacctggtattatactcatatgcgtgatcacacaaattccaatggatgcttttggaattttatatccgaagaacatggttggcaggaaaaataa

SEQ ID NO: 39 - DNA, 3759 bp, Artificial Sequence
  mat_peptide (1)...(3756): BoNT/E, S. cerevisiae-modified 2
atgccaaaaattaattcttttaattataatgatccagttaatgatagaactattttgtatattaaaccaggtggttgtcaagaattttataaatcttttaatattatgaaaaatatttggattattccagaaagaaatgttattggtactactccacaagattttcatccaccaacttctttgaaaaatggtgattcttcttattatgatccaaattatttgcaatctgatgaagaaaaagatagatttttgaaaattgttactaaaatttttaatagaattaataataatttgtctggtggtattttgttggaagaattgtctaaagctaatccatatttgggtaatgataatactccagataatcaatttcatattggtgatgcttctgctgttgaaattaaattttctaatggttctcaagatattttgttgccaaatgttattattatgggtgctgaaccagatttgtttgaaactaattcttctaatatttctttgagaaataattatatgccatctaatcatggttttggttctattgctattgttactttttctccagaatattcttttagatttaatgataattctatgaatgaatttattcaagatccagctttgactttgatgcatgaattgattcattctttgcatggtttgtatggtgctaaaggtattactactaaatatactattactcaaaaacaaaatccattgattactaatattagaggtactaatattgaagaatttttgacttttggtggtactgatttgaatattattacttctgctcaatctaatgatatttatactaatttgttggctgattataaaaaaattgcttctaaattgtctaaagttcaagtttctaatccattgttgaatccatataaagatgtttttgaagctaaatatggtttggataaagatgcttctggtatttattctgttaatattaataaatttaatgatatttttaaaaaattgtattcttttactgaatttgatttggctactaaatttcaagttaaatgtagacaaacttatattggtcaatataaatattttaaattgtctaatttgttgaatgattctatttataatatttctgaaggttataatattaataatttgaaagttaattttagaggtcaaaatgctaatttgaatccaagaattattactccaattactggtagaggtttggttaaaaaaattattagattttgtaaaaatattgtttctgttaaaggtattagaaaatctatttgtattgaaattaataatggtgaattgttttttgttgcttctgaaaattcttataatgatgataatattaatactccaaaagaaattgatgatactgttacttctaataataattatgaaaatgatttggatcaagttattttgaattttaattctgaatctgctccaggtttgtctgatgaaaaattgaatttgactattcaaaatgatgcttatattccaaaatatgattctaatggtacttctgatattgaacaacatgatgttaatgaattgaatgtttttttttatttggatgctcaaaaagttccagaaggtgaaaataatgttaatttgacttcttctattgatactgctttgttggaacaaccaaaaatttatacttttttttcttctgaatttattaataatgttaataaaccagttcaagctgctttgtttgtttcttggattcaacaagttttggttgattttactactgaagctaatcaaaaatctactgttgataaaattgctgatatttctattgttgttccatatattggtttggctttgaatattggtaatgaagctcaaaaaggtaattttaaagatgctttggaattgttgggtgctggtattttgttggaatttgaaccagaattgttgattccaactattttggtttttactattaaatcttttttgggttcttctgataataaaaataaagttattaaagctattaataatgctttgaaagaaagagatgaaaaatggaaagaagtttattcttttattgtttctaattggatgactaaaattaatactcaatttaataaaagaaaagaacaaatgtatcaagctttgcaaaatcaagttaatgctattaaaactattattgaatctaaatataattcttatactttggaagaaaaaaatgaattgactaataaatatgatattaaacaaattgaaaatgaattgaatcaaaaagtttctattgctatgaataatattgatagatttttgactgaatcttctatttcttatttgatgaaattgattaatgaagttaaaattaataaattgagagaatatgatgaaaatgttaaaacttatttgttgaattatattattcaacatggttctattttgggtgaatctcaacaagaattgaattctatggttactgatactttgaataattctattccatttaaattgtcttcttatactgatgataaaattttgatttcttattttaataaattttttaaaagaattaaatcttcttctgttttgaatatgagatataaaaatgataaatatgttgatacttctggttatgattctaatattaatattaatggtgatgtttataaatatccaactaataaaaatcaatttggtatttataatgataaattgtctgaagttaatatttctcaaaatgattatattatttatgataataaatataaaaatttttctatttctttttgggttagaattccaaattatgataataaaattgttaatgttaataatgaatatactattattaattgtatgagagataataattctggttggaaagtttctttgaatcataatgaaattatttggactttgcaagataatgctggtattaatcaaaaattggcttttaattatggtaatgctaatggtatttctgattatattaataaatggatttttgttactattactaatgatagattgggtgattctaaattgtatattaatggtaatttgattgatcaaaaatctattttgaatttgggtaatattcatgtttctgataatattttgtttaaaattgttaattgttcttatactagatatattggtattagatattttaatatttttgataaagaattggatgaaactgaaattcaaactttgtattctaatgaaccaaatactaatattttgaaagatttttggggtaattatttgttgtatgataaagaatattatttgttgaatgttttgaaaccaaataattttattgatagaagaaaagattctactttgtctattaataatattagatctactattttgttggctaatagattgtattctggtattaaagttaaaattcaaagagttaataattcttctactaatgataatttggttagaaaaaatgatcaagtttatattaattttgttgcttctaaaactcatttgtttccattgtatgctgatactgctactactaataaagaaaaaactattaaaatttcttcttctggtaatagatttaatcaagttgttgttatgaattctgttggtaataattgtactatgaattttaaaaataataatggtaataatattggtttgttgggttttaaagctgatactgttgttgcttctacttggtattatactcatatgagagatcatactaattctaatggttgtttttggaattttatttctgaagaacatggttggcaagaaaaataa

SEQ ID NO: 40 - DNA, 3759 bp, Artificial Sequence
  mat_peptide (1)...(3756): BoNT/E, S. cerevisiae-modified 3
atgccaaagataaattcatttaactataatgatcccgtgaatgatcgtacaatactttatattaaaccaggaggttgccaagagttttacaagtctttcaatataatgaagaatatctggataatcccagaaagaaatgtgattggaactacacctcaggattttcatccacccacatcacttaagaatggtgattcttcatattacgatccaaattatttgcaaagcgacgaagagaaggacaggttcttaaaaatagtcactaaaatatttaatagaattaataacaatttgagcggtggaatattactagaagagttgtccaaggctaatccatatttgggtaacgataataccccagataatcaatttcacattggcgatgcttccgctgtggaaatcaagttctcgaacggttctcaagatatactattgcctaatgtgatcattatgggtgctgagccagatttgttcgaaactaatagttctaatatcagtctaaggaataactatatgccatcaaatcatggtttcggttctatcgcaattgtaaccttctcccctgaatattcatttagatttaacgataattcaatgaatgaatttattcaggaccccgccttgacactgatgcatgagttgattcattctttgcacggtctgtacggtgcaaaaggtatcactaccaagtatacaataacgcaaaaacaaaatcctttaattaccaacattagaggtaccaacatcgaagagttcttgacctttggcggaacggatttaaacatcattacgagcgcacaatcgaatgatatttacacaaatctacttgctgattacaagaaaatcgcttcgaagttgagcaaagttcaagtttctaacccattgctgaatccatataaggatgtattcgaggccaaatatggtttagacaaagacgcatccggtatctattcagtcaacattaacaagtttaacgatatttttaagaaattgtattccttcacggagtttgaccttgctacaaagtttcaagtcaagtgcagacagacatatataggccaatataaatacttcaaattgtctaacctattaaatgactctatttacaatatttctgaaggctacaacataaacaatttgaaagttaatttcagaggtcaaaacgcaaatttaaatcccaggatcataacgccaatcacgggacgtggtctggttaaaaagattatcagattttgtaaaaatattgtttctgttaaaggtataagaaaatcaatctgtattgaaataaacaatggtgaactgtttttcgtcgctagtgaaaactcttataatgacgataatataaacacgcctaaagaaattgacgatactgtaacttcgaacaataactatgaaaacgatctagatcaagtgatcctaaacttcaactcggaaagtgctcctggattgtccgacgaaaagttaaaccttacaattcagaacgatgcctatatccctaaatatgactcaaacggaacttcagacatagaacaacatgatgtaaacgaacttaatgtatttttctaccttgacgcacaaaaggttccagagggcgaaaataacgtgaacttaacctcatcgattgataccgcattgcttgaacaaccaaaaatctacacattcttttcttccgagtttattaacaatgttaacaagccagtccaagctgccctattcgtttcttggattcagcaagtgctagttgatttcactacagaggctaatcaaaaatctaccgtagataagatcgccgatatttcaattgtagtcccatatataggacttgccctaaacattggtaacgaagcacaaaaaggtaattttaaggacgccctagagttactgggtgcaggtattttgttagaattcgaaccagaattattgattccaactatattggtctttacgataaagagttttcttggaagcagtgataacaagaataaagttatcaaagctataaataacgccttaaaggaaagggatgaaaaatggaaagaagtgtacagtttcattgtgagcaattggatgactaagattaatactcaatttaataagagaaaagaacagatgtaccaagcattacagaatcaggtaaatgctattaagactataattgaatccaaatacaatagttataccctggaggaaaaaaatgagcttactaacaaatatgatatcaaacagattgaaaatgaattaaaccaaaaagtttccatcgcaatgaataacatagatagattcttaaccgaatcgtctatctcctacctaatgaaacttataaatgaagttaagataaacaaattacgtgaatatgacgaaaacgtcaaaacctacttgctgaactatataatccaacacggttcaatcttgggagaaagccaacaggagttgaattctatggtaaccgacactttgaacaatagtattccttttaaattatcctcttacactgacgataagattttaatctcttattttaacaagtttttcaagagaattaaatcgtcttcggttttaaatatgagatacaaaaatgataaatatgtcgatacgagtggctatgattccaatatcaatataaacggtgatgtatacaaatacccaactaataaaaatcagttcggtatttataatgacaaactgtctgaagtaaatatttcacagaacgattacataatctatgataataagtataagaacttttccatatcattttgggtaaggattcctaattatgacaacaaaatagtgaatgtaaataacgagtacacaatcataaattgcatgagagataataactccggctggaaagtcagtttgaaccataacgaaatcatatggacattgcaggataacgctggcattaatcaaaagttggcctttaactatggtaatgctaatggaatctcagactacattaataagtggatatttgttacaattactaatgatagactgggcgattctaaattgtacataaacggtaatttgattgatcaaaaaagcattttgaacttaggtaacattcacgtttcagataatatattatttaaaattgttaattgtagctacacacgttacatcggtataaggtacttcaatattttcgacaaagaattagacgaaactgagatccaaacactatactctaacgagcccaatacaaatattctaaaggatttttggggtaattacttattgtatgacaaggaatactatttattgaatgttttaaaacctaacaattttattgatagaaggaaagactctacactttccattaataacattagaagtactatcttactggctaacagactatatagtggaattaaagttaagattcagagagtcaataactccagtaccaatgataatttagtgagaaaaaatgaccaagtttatattaacttcgttgcatcaaagactcatttgttccctttgtatgctgatacggctacaaccaataaggaaaagactattaaaattagtagctctggcaatcgttttaatcaagttgtcgtgatgaattcagttggaaataactgtacaatgaactttaaaaataacaatggcaataacattggtttgttgggttttaaagctgatactgtcgttgcttctacttggtattatacacatatgagagaccacactaattcaaatggttgtttttggaattttattagcgaggaacatggttggcaagaaaaataa

SEQ ID NO: 41 - DNA, 3759 bp, Artificial Sequence
  mat_peptide (1)...(3756): BoNT/E, S. pombe-modified 1
atgcccaagataaactcgtttaattataacgacccggttaatgaccgtaccatactttatatcaaaccaggaggttgtcaagaattttataaaagttttaatattatgaaaaatatatggataattccagaacgcaacgttataggtaccacgcctcaagactttcatccccctacgtcgttaaagaacggtgattcatcctattacgatccaaattatctccaatctgatgaagagaaggatcgattcctaaaaattgttactaaaatttttaatcgtattaacaataacctatctggaggtattcttttagaggaattgtctaaagcaaacccttatttaggaaacgacaacacgcctgacaaccagtttcacataggcgatgcctccgcagtggagataaaatttagcaatggatctcaagacatcttacttccgaatgtaataattatgggagccgagccagatttatttgaaactaattcaagtaatatctctttaagaaataactatatgccatccaatcatggctttggttcgatagcaatagttacttttagccctgaatactcattcagatttaacgacaattcaatgaacgaatttattcaggacccagcgttgactttaatgcacgaacttattcactcccttcatggcctctatggagcaaaggggattacaactaaatatacaatcacacaaaagcagaaccccttaattactaacatcaggggtactaatattgaagagttccttactttcggcggtaccgatctaaatattataactagtgctcaaagcaacgatatctatactaatcttctcgccgattataaaaagatcgcatctaaattatccaaagtacaagttagtaatcctcttttgaatccttacaaggacgtatttgaggctaaatatgggctcgataaagatgctagtggaatttattccgttaatataaacaaatttaatgatatttttaagaaactatactctttcactgaatttgatttagccacaaaatttcaagtcaagtgccgtcaaacttatatcggtcaatacaagtattttaaactctctaatttactcaatgattcgatttataatatttctgaaggttacaatattaataacctgaaagttaattttaggggtcaaaatgctaatcttaaccctcgcatcataactcctataactggacgagggttggtcaagaaaataattcgtttttgtaaaaatatcgtttccgttaaaggaattcgtaaatctatttgtatagaaattaacaatggagaattatttttcgtggctagcgagaattcttataatgacgataatattaacacacctaaggaaatcgacgatactgtcacttctaataacaattatgagaacgaccttgatcaagtgatactaaattttaactcagaatctgcacctggattgagtgatgagaagttaaatcttactatacaaaacgatgcttatatcccgaaatatgatagcaatggaacctctgatattgagcagcatgatgtgaacgaattgaatgtgtttttctatttagacgctcaaaaagtacctgaaggtgagaataacgtaaacttaacctcttcgattgataccgctttgcttgaacaacctaaaatttatacatttttcagttcagaattcattaacaatgttaataagcctgttcaagcagctcttttcgtatcatggattcaacaggtccttgtggattttaccactgaggctaaccaaaaatcaacagtagataagattgctgacattagcatagtcgtaccatacatcggccttgcgcttaatattggtaatgaggcacagaaaggaaatttcaaggatgcccttgaattattgggcgctgggattctgttagagtttgaacccgaactgcttattccaaccattcttgtcttcaccatcaaatcttttctaggttcttcagataataagaacaaagttattaaagctataaataacgcattaaaagaacgtgatgaaaaatggaaggaggtgtatagtttcattgtttcaaattggatgacaaagattaatactcaatttaataaaagaaaagaacagatgtaccaagctcttcaaaatcaagttaatgctattaagacaataattgaatctaaatataactcatatacactggaggaaaagaatgaattgactaataaatatgatattaaacaaatcgaaaacgaattaaatcaaaaagttagtattgctatgaataacatagatcgctttttgactgaatctagtatttcctatttaatgaagttaattaatgaggttaagatcaacaaattacgagagtatgatgaaaatgtcaagacgtacttgcttaattatattatccaacatgggtccatccttggtgagtctcagcaagaattgaactcaatggttactgatacattaaataactctatccctttcaaacttagctcatatactgacgataaaattctgatttcttattttaataaatttttcaaacgtattaaaagttcgtcagttcttaatatgcgatacaagaatgataaatacgtcgacacatcgggctatgattcaaatattaacattaatggtgacgtgtataaatatccaactaataaaaaccaatttggtatatacaatgataagttgtctgaggtcaatatttctcagaatgattacatcatttacgacaacaaatacaaaaatttttccatctctttttgggttcgtatcccaaactacgataacaaaatagtcaatgttaataacgaatatacaataattaactgtatgcgagataataactcaggttggaaggtatccctaaatcataacgaaattatctggactttgcaggacaacgctggaattaatcaaaagctcgcttttaattatggtaatgcgaatggtataagtgattacattaataaatggatctttgtaaccattacaaatgacagattaggcgattctaagctttatatcaatggaaatctaattgatcagaaaagtattttgaatcttggtaatattcatgtcagcgataacattttgttcaagattgttaattgctcctacactaggtatattggaatacgttactttaacatctttgataaagagttggatgaaactgaaatacaaacgttatatagcaatgaacctaatacgaatattttgaaagacttttggggcaactacctgttgtatgataaagaatattacttgctaaatgttttgaagcccaacaattttattgatagacggaaagattctaccttgtcgattaataacattcggtctactattctcttagccaatagattgtacagtggaattaaagttaaaattcaaagagttaataactcctctactaatgataatttagttcgcaagaatgatcaagtatatattaattttgttgctagcaagacccacttgttccccctgtacgcagacacggcgacgacaaacaaagaaaagaccatcaaaatttcatcttcaggcaatagatttaatcaggttgtagttatgaactcagtaggtaataactgcacaatgaattttaagaataataatggtaataatattggattattgggttttaaggctgatacagttgttgcctctacttggtattatacccatatgcgtgatcatacaaatagtaatggttgtttttggaatttcatttctgaagaacatggttggcaagaaaagtaa

SEQ ID NO: 42 - DNA, 3759 bp, Artificial Sequence
  mat_peptide (1)...(3756): BoNT/E, S. pombe-modified 2
atgcctaaaattaattcttttaattataatgatcctgttaatgatcgtactattttatatattaaacctggtggttgtcaagaattttataaatcttttaatattatgaaaaatatttggattattcctgaacgtaatgttattggtactactcctcaagattttcatcctcctacttctttaaaaaatggtgattcttcttattatgatcctaattatttacaatctgatgaagaaaaagatcgttttttaaaaattgttactaaaatttttaatcgtattaataataatttatctggtggtattttattagaagaattatctaaagctaatccttatttaggtaatgataatactcctgataatcaatttcatattggtgatgcttctgctgttgaaattaaattttctaatggttctcaagatattttattacctaatgttattattatgggtgctgaacctgatttatttgaaactaattcttctaatatttctttacgtaataattatatgccttctaatcatggttttggttctattgctattgttactttttctcctgaatattcttttcgttttaatgataattctatgaatgaatttattcaagatcctgctttaactttaatgcatgaattaattcattctttacatggtttatatggtgctaaaggtattactactaaatatactattactcaaaaacaaaatcctttaattactaatattcgtggtactaatattgaagaatttttaacttttggtggtactgatttaaatattattacttctgctcaatctaatgatatttatactaatttattagctgattataaaaaaattgcttctaaattatctaaagttcaagtttctaatcctttattaaatccttataaagatgtttttgaagctaaatatggtttagataaagatgcttctggtatttattctgttaatattaataaatttaatgatatttttaaaaaattatattcttttactgaatttgatttagctactaaatttcaagttaaatgtcgtcaaacttatattggtcaatataaatattttaaattatctaatttattaaatgattctatttataatatttctgaaggttataatattaataatttaaaagttaattttcgtggtcaaaatgctaatttaaatcctcgtattattactcctattactggtcgtggtttagttaaaaaaattattcgtttttgtaaaaatattgtttctgttaaaggtattcgtaaatctatttgtattgaaattaataatggtgaattattttttgttgcttctgaaaattcttataatgatgataatattaatactcctaaagaaattgatgatactgttacttctaataataattatgaaaatgatttagatcaagttattttaaattttaattctgaatctgctcctggtttatctgatgaaaaattaaatttaactattcaaaatgatgcttatattcctaaatatgattctaatggtacttctgatattgaacaacatgatgttaatgaattaaatgtttttttttatttagatgctcaaaaagttcctgaaggtgaaaataatgttaatttaacttcttctattgatactgctttattagaacaacctaaaatttatacttttttttcttctgaatttattaataatgttaataaacctgttcaagctgctttatttgtttcttggattcaacaagttttagttgattttactactgaagctaatcaaaaatctactgttgataaaattgctgatatttctattgttgttccttatattggtttagctttaaatattggtaatgaagctcaaaaaggtaattttaaagatgctttagaattattaggtgctggtattttattagaatttgaacctgaattattaattcctactattttagtttttactattaaatcttttttaggttcttctgataataaaaataaagttattaaagctattaataatgctttaaaagaacgtgatgaaaaatggaaagaagtttattcttttattgtttctaattggatgactaaaattaatactcaatttaataaacgtaaagaacaaatgtatcaagctttacaaaatcaagttaatgctattaaaactattattgaatctaaatataattcttatactttagaagaaaaaaatgaattaactaataaatatgatattaaacaaattgaaaatgaattaaatcaaaaagtttctattgctatgaataatattgatcgttttttaactgaatcttctatttcttatttaatgaaattaattaatgaagttaaaattaataaattacgtgaatatgatgaaaatgttaaaacttatttattaaattatattattcaacatggttctattttaggtgaatctcaacaagaattaaattctatggttactgatactttaaataattctattccttttaaattatcttcttatactgatgataaaattttaatttcttattttaataaattttttaaacgtattaaatcttcttctgttttaaatatgcgttataaaaatgataaatatgttgatacttctggttatgattctaatattaatattaatggtgatgtttataaatatcctactaataaaaatcaatttggtatttataatgataaattatctgaagttaatatttctcaaaatgattatattatttatgataataaatataaaaatttttctatttctttttgggttcgtattcctaattatgataataaaattgttaatgttaataatgaatatactattattaattgtatgcgtgataataattctggttggaaagtttctttaaatcataatgaaattatttggactttacaagataatgctggtattaatcaaaaattagcttttaattatggtaatgctaatggtatttctgattatattaataaatggatttttgttactattactaatgatcgtttaggtgattctaaattatatattaatggtaatttaattgatcaaaaatctattttaaatttaggtaatattcatgtttctgataatattttatttaaaattgttaattgttcttatactcgttatattggtattcgttattttaatatttttgataaagaattagatgaaactgaaattcaaactttatattctaatgaacctaatactaatattttaaaagatttttggggtaattatttattatatgataaagaatattatttattaaatgttttaaaacctaataattttattgatcgtcgtaaagattctactttatctattaataatattcgttctactattttattagctaatcgtttatattctggtattaaagttaaaattcaacgtgttaataattcttctactaatgataatttagttcgtaaaaatgatcaagtttatattaattttgttgcttctaaaactcatttatttcctttatatgctgatactgctactactaataaagaaaaaactattaaaatttcttcttctggtaatcgttttaatcaagttgttgttatgaattctgttggtaataattgtactatgaattttaaaaataataatggtaataatattggtttattaggttttaaagctgatactgttgttgcttctacttggtattatactcatatgcgtgatcatactaattctaatggttgtttttggaattttatttctgaagaacatggttggcaagaaaaataa

SEQ ID NO: 43 - DNA, 3759 bp, Artificial Sequence
  mat_peptide (1)...(3756): BoNT/E, S. pombe-modified 3
atgcctaaaatcaactcatttaactacaatgatccagtgaacgatagaactatcttatatataaaaccaggtggatgtcaagaattttataagtcgttcaatataatgaagaatatatggattatccctgaacgtaatgttattggaaccacaccacaagactttcaccctccaacttctttaaaaaatggcgattcctcatactatgatcctaattatttgcaatccgatgaagagaaagaccgcttcctaaaaatagttactaaaatttttaatagaattaacaataacttaagtggaggtattcttttggaggaattgagtaaagctaacccttatcttggaaatgataatacacccgataatcagtttcatatcggtgatgctagtgcagtggaaattaagttctctaacggatctcaagatatcttgcttcccaatgttataattatgggagctgagccagacttatttgaaaccaactcctctaatataagcttacgtaataactatatgccttccaaccatggtttcggtagcatcgctattgttacattttcacccgaatatagttttcgttttaatgataatagtatgaatgaatttatccaagatccagccttgactttaatgcatgagttaattcattcattacatggtttatacggtgcaaagggcattactacaaagtacactattacccaaaaacaaaatcccttgattactaatattagaggtactaatattgaggaatttttaacatttggtggcactgatttgaacatcattacttcagctcaatcaaatgatatttatactaatttgttagcagactataaaaagattgcttctaaattaagtaaagtacaagtttccaatcctcttttaaatccttataaagatgtctttgaagctaagtatggtttggataaagatgcctctggaatttactctgttaatataaacaagtttaatgatatttttaagaaattatattcgttcactgaattcgatctagcaactaaatttcaagttaaatgtaggcaaacttatattggtcaatataaatattttaagttatctaatttattgaatgactcaatatataacatatctgagggttataacataaataacttgaaagtcaactttcgtggacaaaacgcaaatctaaatcctagaataattactccaattactggtcgtggacttgtgaagaaaattatccgattttgcaagaacattgtaagcgttaagggtatccgtaagtcgatatgcattgagataaataacggtgagctttttttcgtcgcctctgaaaacagttataacgatgacaatataaatacacctaaagagatagatgacactgtgactagcaacaataactacgaaaacgacttagatcaggttattttaaactttaattcggaatctgctccaggattatctgatgaaaaacttaatcttactatacagaacgatgcctacattcctaagtatgactcgaatggtacctccgacatcgagcagcacgatgttaatgaattaaatgtttttttctatcttgatgcacaaaaagttcccgaaggagaaaataacgtcaatttgactagctctattgacactgctttgctagagcaacctaagatttatacctttttcagttcagaatttattaataacgttaataaacctgtacaagccgcattatttgtttcttggattcagcaagtgttggtagactttactaccgaagcaaaccaaaaatctacagttgacaagatcgcagatatttcaattgttgtaccatatattggtttagctttgaatattggaaatgaagctcaaaaaggaaattttaaagacgcccttgagttattgggcgcaggtattttattggaatttgagcctgaattacttatccctactattttagtttttacaataaaaagcttccttggatcttcagataataagaataaagtcattaaagccatcaataacgctttaaaggaaagagacgaaaaatggaaagaagtctactcatttatagtgagtaattggatgactaagattaacactcaattcaataaacgcaaagaacaaatgtatcaagctttacagaatcaggtaaatgctattaagactatcattgagtccaaatataattcttatacacttgaagagaaaaatgaattgactaataagtacgacatcaaacaaatcgaaaacgagttgaatcaaaaggtttccattgctatgaataacattgatcgtttccttacagaatcctctatctcatacttgatgaaattaatcaatgaggtaaaaatcaataagcttcgtgaatatgatgaaaacgtcaaaacttatcttctaaattatattatccagcatggttcaattttaggtgagtcccaacaggagcttaatagcatggtcaccgacactcttaacaatagcattccttttaagttatcatcttataccgacgataaaattttaatttcatatttcaacaagtttttcaaaaggattaaatcaagttctgttttgaatatgagatataaaaatgataaatacgttgatacaagtggttatgattctaatattaacattaatggcgatgtttataaatatcctaccaataaaaatcaatttggcatttataatgataaactttccgaagtaaatatttctcaaaatgattacattatctacgataataagtataaaaatttcagtatttccttttgggtaaggattccaaattacgataataaaattgttaacgtaaacaatgagtataccataattaattgtatgcgtgacaacaatagtggttggaaagtttcgctaaatcacaatgaaataatttggactttacaagataatgctggaataaatcaaaagttagcttttaactacggtaatgctaatggtatatctgattacatcaacaaatggatttttgtgacaattacaaatgatcgattgggcgattcaaaattatacattaacggtaacctaattgatcagaagagcattttaaaccttggtaacattcatgtcagtgataatatactatttaaaatagtaaattgctcttatacacgttatattggaattcgttacttcaatatattcgataaagaattagatgaaacagaaatccagactttatattctaacgaacccaacaccaatatcttgaaggatttttggggaaattaccttttatatgataaggaatactatcttttaaatgtgcttaagcctaataacttcattgatagacgaaaggactctacacttagtattaataacattcgatcaaccattcttttagctaatcgactatattctggtatcaaagttaagattcaacgcgttaataactcttcgactaacgataatttggtaagaaaaaatgatcaagtctatattaattttgttgcttcgaagactcatctttttcctttatacgctgacactgctacaaccaataaagaaaaaactatcaagatatctagctctggtaatcgctttaatcaggtagttgtcatgaattctgttggtaataactgtactatgaattttaaaaacaataacggaaataacataggtttgttaggctttaaggctgatactgttgttgcttccacatggtactacacccacatgcgtgaccatactaattctaatggctgtttttggaactttatatcagaagagcatggctggcaagaaaaataa

SEQ ID NO: 44 - DNA, 3759 bp, Artificial Sequence
  mat_peptide (1)...(3756): BoNT/E, Y. lipolytica-modified 1
atgcccaagatcaacagttttaactacaacgatcctgttaacgaccgaactattctctacatcaaacccggtggctgtcaggagttttacaagtctttcaatattatgaagaacatctggatcattcccgagagaaacgtgattgggaccacaccgcaggatttccacccccctacctcactgaagaacggagactctagctactatgaccccaactaccttcagagcgacgaggaaaaggatcggtttctcaagatcgtgaccaagatctttaaccgtattaacaataacttgtcaggtgggattctcctagaggaattgagcaaggctaacccttatcttggtaatgacaacactcccgacaaccagtttcacatcggagacgcttctgccgttgagattaagttttctaacggctcccaggatattctccttcctaacgtcatcattatgggcgccgagcctgatctgtttgagactaattcttcgaacatttcactccgtaacaattacatgccctccaaccacggcttcgggtcaatagccatcgtaaccttttcaccagagtactctttccgattcaacgacaactccatgaacgagttcatccaggatccagccctgaccctcatgcatgagctgatccattcacttcacggtctgtacggtgcgaaaggcatcactaccaagtacacaatcacccagaaacaaaatcctctcattaccaatattcgaggaaccaacattgaggaattccttacattcggtggcaccgatttgaatattatcaccagtgcccagtcgaacgacatctacacgaacctgctcgctgactacaagaaaatcgcttccaagctttcgaaagttcaggtgagcaaccctctactgaatccctacaaggacgtctttgaagccaagtatggcttggacaaggatgcatctggcatttacagcgttaatatcaataagttcaacgatattttcaagaaactttactcttttactgagtttgatttggccaccaagtttcaggtcaagtgtcgacagacgtacatcggacaatataagtacttcaaactctcgaacctccttaacgatagtatttacaacatttccgaaggctacaacatcaataacctcaaggtgaacttcagaggtcagaacgcgaacctcaacccccggatcattacccctattacaggccggggccttgtcaagaaaattatacgattttgcaagaacatcgtcagcgtgaaaggcattcgtaagtccatctgcatagagattaacaatggcgagttattctttgtcgcctccgagaactcgtacaatgacgataacatcaacactcccaaggaaatcgatgacacagtgacatctaacaataactacgaaaacgacctggatcaggttatcctgaacttcaattccgagtctgctcccggtctgtctgatgagaagctcaaccttactattcagaatgatgcctacatcccaaagtacgactcgaacggaacctcggacatcgaacagcacgacgtgaacgagctgaatgtctttttctacctcgacgcgcagaaggtcccggagggagaaaacaatgtgaaccttacgtccagcatcgatactgcacttttggagcaacccaagatctatacttttttctctagcgagttcattaacaatgttaacaaacccgtccaagctgccctgtttgtgtcctggattcagcaagtactcgtcgactttaccactgaggcaaaccaaaaatcgaccgtggacaagatcgctgacatttccattgtggtcccttatattggactggctctcaacattggaaacgaagcgcagaagggaaactttaaggacgctttggagctgctcggagcaggaatcctcctggaatttgaaccagagctactgattcctacaatcctcgtattcaccatcaaaagtttcttaggctcctctgacaacaaaaacaaggtgatcaaggctatcaacaatgcactgaaagagcgggatgagaagtggaaggaagtttactcgttcattgtgtccaactggatgacaaagattaacacacaatttaacaagcgcaaggagcagatgtaccaagctctgcagaatcaggtgaacgcgatcaagaccattatcgagtcaaagtataactcttataccctggaggaaaaaaacgagctcaccaacaagtacgacatcaagcagattgagaacgagctgaaccagaaggtctccattgccatgaacaatattgaccgattcttgaccgagtcttcgatctcctacctcatgaagctgatcaacgaggtcaaaattaacaagctgcgggaatatgacgaaaacgttaagacttacttgctgaactacattatccagcatggttccatcctgggcgagtcccagcaagagctgaactccatggtgaccgacactcttaataactctattcctttcaagctgtcttcctacacagacgataagatcctgatctcgtacttcaacaaatttttcaagagaattaagtcctctagtgtcttgaatatgcgctacaagaacgacaagtacgtcgacactagcggctacgattctaacattaacattaacggagacgtgtacaagtaccccactaataagaaccagttcggcatctacaatgacaagctctctgaagtgaacatctcgcaaaacgactacatcatttacgacaacaagtacaagaatttttctatcagcttctgggttcgcatcccgaactacgataataagatcgtgaatgtcaacaatgagtatacgatcataaactgtatgcgagacaacaattccggatggaaggtgtcactcaaccacaacgagatcatttggaccttgcaggacaacgccggtattaaccagaagctagccttcaactatgggaacgccaacggaatttccgattacattaacaagtggatattcgttaccatcacgaacgatagactgggcgactcaaaactgtacatcaacggaaacctaatcgatcagaagtccattctcaacctgggtaatattcatgtctctgacaacatccttttcaagatcgtcaactgctcttatacgagatacatcggtatccgatactttaatattttcgataaggagctggatgagaccgagattcagactctctactcgaacgagcccaacaccaatatcctgaaggacttctggggcaactatctgctttatgataaggagtactatctgctcaacgttctcaaaccaaacaatttcattgaccgtcgaaaggacagcacactgagcatcaacaatattcgatcgaccatcctgttggcaaacaggctgtactcgggaatcaaggttaagattcagcgagtgaacaactccagtacgaacgacaaccttgtgcgaaagaacgaccaggtttacattaacttcgtggcttctaagacccacctattcccgctgtacgccgacaccgctacgactaacaaggagaaaacgatcaagatttcttcgagtggaaaccgattcaaccaggtcgttgtaatgaactctgtgggaaacaactgtacgatgaactttaagaacaacaacggcaacaacattggtctgctgggttttaaggccgacacagtggtcgccagtacttggtactacactcatatgcgagaccacactaactcgaacggatgcttctggaacttcatttcggaggaacatggttggcaggagaagtaa

SEQ ID NO: 45 - DNA, 3759 bp, Artificial Sequence
  mat_peptide (1)...(3756): BoNT/E, Y. lipolytica-modified 2
atgcccaagatcaactctttcaactacaacgaccccgtgaacgaccgaaccatcctgtacatcaagcccggcggctgtcaggagttctacaagtctttcaacatcatgaagaacatctggatcatccccgagcgaaacgtgatcggcaccaccccccaggacttccacccccccacctctctgaagaacggcgactcttcttactacgaccccaactacctgcagtctgacgaggagaaggaccgattcctgaagatcgtgaccaagatcttcaaccgaatcaacaacaacctgtctggcggcatcctgctggaggagctgtctaaggccaacccctacctgggcaacgacaacacccccgacaaccagttccacatcggcgacgcctctgccgtggagatcaagttctctaacggctctcaggacatcctgctgcccaacgtgatcatcatgggcgccgagcccgacctgttcgagaccaactcttctaacatctctctgcgaaacaactacatgccctctaaccacggcttcggctctatcgccatcgtgaccttctctcccgagtactctttccgattcaacgacaactctatgaacgagttcatccaggaccccgccctgaccctgatgcacgagctgatccactctctgcacggcctgtacggcgccaagggcatcaccaccaagtacaccatcacccagaagcagaaccccctgatcaccaacatccgaggcaccaacatcgaggagttcctgaccttcggcggcaccgacctgaacatcatcacctctgcccagtctaacgacatctacaccaacctgctggccgactacaagaagatcgcctctaagctgtctaaggtgcaggtgtctaaccccctgctgaacccctacaaggacgtgttcgaggccaagtacggcctggacaaggacgcctctggcatctactctgtgaacatcaacaagttcaacgacatcttcaagaagctgtactctttcaccgagttcgacctggccaccaagttccaggtgaagtgtcgacagacctacatcggccagtacaagtacttcaagctgtctaacctgctgaacgactctatctacaacatctctgagggctacaacatcaacaacctgaaggtgaacttccgaggccagaacgccaacctgaacccccgaatcatcacccccatcaccggccgaggcctggtgaagaagatcatccgattctgtaagaacatcgtgtctgtgaagggcatccgaaagtctatctgtatcgagatcaacaacggcgagctgttcttcgtggcctctgagaactcttacaacgacgacaacatcaacacccccaaggagatcgacgacaccgtgacctctaacaacaactacgagaacgacctggaccaggtgatcctgaacttcaactctgagtctgcccccggcctgtctgacgagaagctgaacctgaccatccagaacgacgcctacatccccaagtacgactctaacggcacctctgacatcgagcagcacgacgtgaacgagctgaacgtgttcttctacctggacgcccagaaggtgcccgagggcgagaacaacgtgaacctgacctcttctatcgacaccgccctgctggagcagcccaagatctacaccttcttctcttctgagttcatcaacaacgtgaacaagcccgtgcaggccgccctgttcgtgtcttggatccagcaggtgctggtggacttcaccaccgaggccaaccagaagtctaccgtggacaagatcgccgacatctctatcgtggtgccctacatcggcctggccctgaacatcggcaacgaggcccagaagggcaacttcaaggacgccctggagctgctgggcgccggcatcctgctggagttcgagcccgagctgctgatccccaccatcctggtgttcaccatcaagtctttcctgggctcttctgacaacaagaacaaggtgatcaaggccatcaacaacgccctgaaggagcgagacgagaagtggaaggaggtgtactctttcatcgtgtctaactggatgaccaagatcaacacccagttcaacaagcgaaaggagcagatgtaccaggccctgcagaaccaggtgaacgccatcaagaccatcatcgagtctaagtacaactcttacaccctggaggagaagaacgagctgaccaacaagtacgacatcaagcagatcgagaacgagctgaaccagaaggtgtctatcgccatgaacaacatcgaccgattcctgaccgagtcttctatctcttacctgatgaagctgatcaacgaggtgaagatcaacaagctgcgagagtacgacgagaacgtgaagacctacctgctgaactacatcatccagcacggctctatcctgggcgagtctcagcaggagctgaactctatggtgaccgacaccctgaacaactctatccccttcaagctgtcttcttacaccgacgacaagatcctgatctcttacttcaacaagttcttcaagcgaatcaagtcttcttctgtgctgaacatgcgatacaagaacgacaagtacgtggacacctctggctacgactctaacatcaacatcaacggcgacgtgtacaagtaccccaccaacaagaaccagttcggcatctacaacgacaagctgtctgaggtgaacatctctcagaacgactacatcatctacgacaacaagtacaagaacttctctatctctttctgggtgcgaatccccaactacgacaacaagatcgtgaacgtgaacaacgagtacaccatcatcaactgtatgcgagacaacaactctggctggaaggtgtctctgaaccacaacgagatcatctggaccctgcaggacaacgccggcatcaaccagaagctggccttcaactacggcaacgccaacggcatctctgactacatcaacaagtggatcttcgtgaccatcaccaacgaccgactgggcgactctaagctgtacatcaacggcaacctgatcgaccagaagtctatcctgaacctgggcaacatccacgtgtctgacaacatcctgttcaagatcgtgaactgttcttacacccgatacatcggcatccgatacttcaacatcttcgacaaggagctggacgagaccgagatccagaccctgtactctaacgagcccaacaccaacatcctgaaggacttctggggcaactacctgctgtacgacaaggagtactacctgctgaacgtgctgaagcccaacaacttcatcgaccgacgaaaggactctaccctgtctatcaacaacatccgatctaccatcctgctggccaaccgactgtactctggcatcaaggtgaagatccagcgagtgaacaactcttctaccaacgacaacctggtgcgaaagaacgaccaggtgtacatcaacttcgtggcctctaagacccacctgttccccctgtacgccgacaccgccaccaccaacaaggagaagaccatcaagatctcttcttctggcaaccgattcaaccaggtggtggtgatgaactctgtgggcaacaactgtaccatgaacttcaagaacaacaacggcaacaacatcggcctgctgggcttcaaggccgacaccgtggtggcctctacctggtactacacccacatgcgagaccacaccaactctaacggctgtttctggaacttcatctctgaggagcacggctggcaggagaagtaa

SEQ ID NO: 46 - DNA, 3759 bp, Artificial Sequence
  mat_peptide (1)...(3756): BoNT/E, Y. lipolytica-modified 3
atgcctaagattaactctttcaactacaacgatcccgttaacgaccgcactatcctgtacatcaagccaggtggatgccaggagttctacaagtcgtttaacatcatgaagaacatctggatcattcccgagcgaaacgtgattggcactacgcctcaggacttccacccacctacgtccctcaagaacggcgattcttcgtactacgaccctaactacctgcagagcgatgaggaaaaggaccgctttctcaagatcgttactaagatctttaaccgcatcaacaacaacctgtcgggcggaatccttctggaggaactgagcaaggcaaacccttacctcggaaacgacaacacccccgacaaccagttccacatcggagacgcatccgctgtcgaaattaagttttcaaacggatcgcaggacattctgcttcccaacgtgatcattatgggtgccgaacccgacttgtttgagactaactcttccaacatctccctgagaaacaactacatgccctctaaccacggattcggatctatcgccattgtgacgttctcgcctgagtactcgttccgatttaacgacaactctatgaacgagtttatccaggaccccgcattgactctgatgcatgagcttattcattccctgcatggcctttacggcgcaaagggtattacgactaagtacactattactcagaagcagaaccccctgatcaccaacatccggggcactaacatcgaggaattcctgaccttcggaggcaccgacctcaacattatcacatccgcccagtctaacgacatctacacaaacctcctggctgattacaagaagatcgcttctaagctgtctaaggttcaggtgtctaaccccctgttgaacccctacaaggacgtgttcgaggccaagtacggcttggataaggacgccagcggtatttactccgtcaacatcaacaagtttaacgatatttttaagaagctctactctttcacagagttcgacctcgccactaagtttcaggtgaagtgccgtcagacctacattggtcagtacaagtacttcaagctgtctaacctgcttaacgactctatctacaacatttctgagggctacaacattaacaacctcaaggttaacttccgtggccagaacgcaaaccttaacccacgaatcattacccctatcacgggacgaggcctggtgaagaagatcattcggttttgtaagaacatcgtctctgtgaagggtattcggaagtcaatctgcattgagatcaacaacggtgaacttttctttgtggcctctgaaaactcttacaacgacgataacattaacaccccgaaggagattgacgatactgtcacatccaacaacaactacgagaacgacctggatcaggtcatcttgaacttcaactctgaatccgccccgggcctctctgatgagaagcttaacctgaccatccagaacgatgcctacattcctaagtacgattctaacggcacctcagatattgagcagcacgatgtcaacgaactcaacgtcttcttttacctcgacgctcagaaggtgcctgaaggtgagaacaacgtcaaccttacgtcgagcatcgataccgcccttctcgagcagcccaagatttacacctttttctcctcggagttcatcaacaacgtgaacaagcccgtgcaggccgctctgttcgtgtcttggattcagcaggttctggtcgactttacaaccgaggccaaccagaagtcaaccgtggacaagatcgccgatatctctatcgtcgttccttacatcggactggctcttaacattggcaacgaggctcagaagggaaacttcaaggatgctttggaactcctgggtgccggaatcctgctcgagtttgagcctgagttgctcatccccaccatcctggtttttacaatcaagagcttccttggcagctctgataacaagaacaaggttatcaaggccattaacaacgctcttaaggaacgggacgagaagtggaaggaggtgtactcttttattgtttcgaactggatgacaaagattaacacccagtttaacaagagaaaggagcagatgtaccaggcactgcagaaccaggttaacgccatcaagacgatcattgagagcaagtacaactcttacaccctcgaggaaaagaacgagctgaccaacaagtacgacattaagcagatcgaaaacgagctcaaccagaaggtctccattgctatgaacaacattgaccgattcctgactgaatcttcgatcagctaccttatgaagctcatcaacgaggtcaagatcaacaagctgcgagagtacgacgagaacgtcaagacatacctcctgaactacatcattcagcatggctcaattctgggagagtcgcagcaggaactcaactccatggtcacagacacactcaacaacagcatcccattcaagctgtcgtcctacacggacgataagatcctgatttcctacttcaacaagttctttaagagaatcaagtcgtcctctgtgttgaacatgcggtacaagaacgacaagtacgtggatacctcaggctacgattctaacatcaacattaacggtgacgtttacaagtaccccactaacaagaaccagttcggtatctacaacgacaagctgtcggaagtgaacatctcgcagaacgattacatcatttacgacaacaagtacaagaacttctcaattagcttctgggtgcgaatcccgaactacgacaacaagattgtgaacgtcaacaacgaatacactattatcaactgtatgcgagataacaactctggatggaaggtgtcactgaaccataacgagattatctggaccctgcaggacaacgccggtattaaccagaagctggccttcaactacggcaacgctaacggtatctccgattacattaacaagtggatttttgtgacgatcaccaacgaccgactcggcgattctaagctgtacattaacggcaacctgattgaccagaagtcgattctgaacctgggcaacattcacgtttccgacaacatcttgtttaagatcgtcaactgttcctacaccagatacatcggaatccgatacttcaacattttcgacaaggagctggacgagactgagattcagacgctgtactccaacgagccaaacacaaacattctgaaggacttctggggaaactacctgctttacgacaaggagtactacctcctgaacgtcctcaagccgaacaactttatcgaccgaagaaaggactccaccctttcgatcaacaacatccgatctaccattttgctggccaaccgtctctacagcggcattaaggtgaagattcagcgagtgaacaactcctctactaacgataacttggtgcgaaagaacgaccaggtgtacattaactttgtcgcctccaagacacacttgtttcccctgtacgctgatactgctaccactaacaaggagaagaccattaagatctcatcgtccggcaaccgatttaaccaggttgtggtcatgaactctgtcggaaacaactgtaccatgaacttcaagaacaacaacggaaacaacatcggcctcctcggattcaaggccgacaccgttgtggccagcacctggtactacactcatatgcgtgaccacaccaactctaacggttgcttctggaacttcatttccgaggagcacggttggcaggagaagtaa

SEQ ID NO: 47 - DNA, 3759 bp, Artificial Sequence
  mat_peptide (1)...(3756): BoNT/E, D. discoideum-modified 1
atgccaaaaattaattcttttaactataatgatcctgttaatgatagaacaatattatatattaaaccaggtggctgtcaagaattttataaatcattcaatattatgaaaaatatatggattataccagaaagaaatgttatcggtacaacgccacaagatttccatcctccaacctcattaaagaatggtgattcaagttattacgatccaaattatttacaatcagatgaggaaaaagatagatttttgaaaattgttacaaagatttttaatagaattaataacaatttaagtggtggcatattgcttgaagagttatctaaagccaatccatatttaggtaatgataatacaccagacaatcaatttcatattggtgatgcatcagctgtagaaattaaattctctaatggtagccaagatatcttattgccaaatgttataattatgggtgcagaacccgatttatttgaaacaaattcttcaaatatatcattaagaaataactacatgcctagtaatcatggatttggttctattgctattgtgacatttagtccagagtattcttttcgttttaatgataattcaatgaatgaattcattcaggatccagcacttactttaatgcatgaattgattcatagcttacatggtttatatggagctaaaggtatcactacaaaatatacaattacccaaaaacaaaatccattaatcacaaatattagaggaactaatattgaagagtttctaacctttggtggaacagatctaaatataattacttctgctcaatcaaatgatatttatacaaatctattagctgattataaaaagatcgcatctaaattatcaaaagtacaagtttcaaatccattacttaatccatataaggatgtattcgaagcaaaatatggtttagataaagatgcttcaggtatttattcagttaatattaataaatttaatgatatttttaagaaattatactctttcaccgaatttgatctagcaacaaaatttcaagttaaatgtagacaaacttatattggacaatataaatattttaaattgtcaaatttattgaatgattcaatatataatattagtgaaggttataatattaataacttaaaggtcaattttagaggtcaaaatgccaatttaaatccaagaataattactccaattacaggcagaggactagtaaaaaagattatacgtttctgtaaaaatattgtctctgttaaaggtataagaaaatcaatttgtattgaaattaacaatggagaattatttttcgttgcttcagaaaattcatataatgatgacaatatcaatactcctaaagaaattgatgacacagtaacttcaaataacaattatgaaaatgatcttgatcaagtgattcttaattttaattcggaatctgcaccaggattatcagatgaaaaattaaatcttacaattcaaaatgatgcctatattccaaaatatgatagtaatggtacaagtgatatagaacaacatgatgttaatgaattaaatgttttcttttacttagatgcacagaaagtcccagaaggtgaaaataacgttaatttgacatcaagtatcgatacagcattattggaacaaccaaaaatatatacatttttctcctcagaattcattaataacgtaaataaaccagttcaagcagctttatttgtttcatggatccaacaagtactcgttgattttacaaccgaggctaatcaaaaatcaactgttgataaaatagcagacattagtatagttgtaccatacattggtttagctttaaatattggtaatgaagcccaaaagggtaattttaaagatgcattagagttgttaggtgcaggtattttacttgagtttgaacctgaacttttaattccaactattctcgttttcacaataaaaagttttttaggttcaagtgataataaaaataaagttattaaagccataaataacgcattaaaagaaagagatgaaaagtggaaagaagtttattcatttattgtatcaaattggatgactaaaattaacacacaattcaataaacgtaaagaacaaatgtatcaagcattgcaaaatcaagtaaatgctattaaaaccatcattgaatcaaaatataattcttatacacttgaagagaaaaatgaattgacaaataaatacgatattaaacaaattgaaaatgaattaaatcaaaaagtttcaattgctatgaataacatcgatagattcttaactgaatcgagtatttcatatttaatgaagttaattaatgaagttaagattaataaacttagagaatatgatgaaaatgtaaaaacttatttattgaattatataatccaacatggtagtatcttaggtgaaagtcaacaagaattaaattcaatggttactgatacattaaataactcaattccatttaaattatctagttatactgatgacaaaattcttatatcatacttcaataaatttttcaaacgtatcaaatcaagctcagtattaaatatgagatataagaatgataaatatgttgatacatccggttacgattcaaatattaatattaatggtgatgtatataaatatccaacaaataaaaatcaatttggtatttataatgataagttatccgaggtcaatattagtcaaaatgattatattatatatgataataaatataaaaatttttctatttccttctgggtaagaatcccaaattatgataataaaatagttaacgtaaataacgaatataccattataaattgtatgagagataataactcaggttggaaagtgtcattaaatcataatgaaataatttggactttacaagataatgctgggattaatcaaaaattagcatttaattatggtaatgcaaatggtatcagtgattacattaataaatggatatttgttactattacaaatgatagattaggtgatagtaaactatatattaatggtaatttaattgaccaaaaaagtattctcaatttaggtaatattcatgtttcagataatattttatttaagattgtaaattgctcatataccagatatattggtatcagatatttcaatatatttgataaagaattagatgaaacagaaatacaaacattatattctaatgaaccaaatactaatattttaaaagatttttggggtaattatttgttatatgataaagaatattaccttttaaatgttttaaaaccaaataacttcattgatagacgtaaagattcaacattatcaattaataacatacgttcaactatattattggcgaataggttatatagtggaattaaagttaagattcaacgtgttaataattcaagtacaaatgataatctcgtacgtaaaaatgatcaagtttatattaattttgtcgcaagtaaaacacatttatttccattatatgcagatactgcaaccactaataaagagaaaaccatcaaaatatcttcatcaggtaatcgattcaatcaagttgtagttatgaattcagttggaaataattgtacaatgaattttaagaataataatggtaataatatcggtctcttaggttttaaagcagatactgtagttgcttcaacttggtattatactcacatgagagatcacactaattcaaatggttgtttttggaattttatatcagaagaacatggttggcaagaaaaataa

SEQ ID NO: 48 - DNA, 3759 bp, Artificial Sequence
  mat_peptide (1)...(3756): BoNT/E, D. discoideum-modified 2
atgccaaaaattaattcatttaattataatgatccagttaatgatagaacaattttatatattaaaccaggtggttgtcaagaattttataaatcatttaatattatgaaaaatatttggattattccagaaagaaatgttattggtacaacaccacaagattttcatccaccaacatcattaaaaaatggtgattcatcatattatgatccaaattatttacaatcagatgaagaaaaagatagatttttaaaaattgttacaaaaatttttaatagaattaataataatttatcaggtggtattttattagaagaattatcaaaagcaaatccatatttaggtaatgataatacaccagataatcaatttcatattggtgatgcatcagcagttgaaattaaattttcaaatggttcacaagatattttattaccaaatgttattattatgggtgcagaaccagatttatttgaaacaaattcatcaaatatttcattaagaaataattatatgccatcaaatcatggttttggttcaattgcaattgttacattttcaccagaatattcatttagatttaatgataattcaatgaatgaatttattcaagatccagcattaacattaatgcatgaattaattcattcattacatggtttatatggtgcaaaaggtattacaacaaaatatacaattacacaaaaacaaaatccattaattacaaatattagaggtacaaatattgaagaatttttaacatttggtggtacagatttaaatattattacatcagcacaatcaaatgatatttatacaaatttattagcagattataaaaaaattgcatcaaaattatcaaaagttcaagtttcaaatccattattaaatccatataaagatgtttttgaagcaaaatatggtttagataaagatgcatcaggtatttattcagttaatattaataaatttaatgatatttttaaaaaattatattcatttacagaatttgatttagcaacaaaatttcaagttaaatgtagacaaacatatattggtcaatataaatattttaaattatcaaatttattaaatgattcaatttataatatttcagaaggttataatattaataatttaaaagttaattttagaggtcaaaatgcaaatttaaatccaagaattattacaccaattacaggtagaggtttagttaaaaaaattattagattttgtaaaaatattgtttcagttaaaggtattagaaaatcaatttgtattgaaattaataatggtgaattattttttgttgcatcagaaaattcatataatgatgataatattaatacaccaaaagaaattgatgatacagttacatcaaataataattatgaaaatgatttagatcaagttattttaaattttaattcagaatcagcaccaggtttatcagatgaaaaattaaatttaacaattcaaaatgatgcatatattccaaaatatgattcaaatggtacatcagatattgaacaacatgatgttaatgaattaaatgtttttttttatttagatgcacaaaaagttccagaaggtgaaaataatgttaatttaacatcatcaattgatacagcattattagaacaaccaaaaatttatacatttttttcatcagaatttattaataatgttaataaaccagttcaagcagcattatttgtttcatggattcaacaagttttagttgattttacaacagaagcaaatcaaaaatcaacagttgataaaattgcagatatttcaattgttgttccatatattggtttagcattaaatattggtaatgaagcacaaaaaggtaattttaaagatgcattagaattattaggtgcaggtattttattagaatttgaaccagaattattaattccaacaattttagtttttacaattaaatcatttttaggttcatcagataataaaaataaagttattaaagcaattaataatgcattaaaagaaagagatgaaaaatggaaagaagtttattcatttattgtttcaaattggatgacaaaaattaatacacaatttaataaaagaaaagaacaaatgtatcaagcattacaaaatcaagttaatgcaattaaaacaattattgaatcaaaatataattcatatacattagaagaaaaaaatgaattaacaaataaatatgatattaaacaaattgaaaatgaattaaatcaaaaagtttcaattgcaatgaataatattgatagatttttaacagaatcatcaatttcatatttaatgaaattaattaatgaagttaaaattaataaattaagagaatatgatgaaaatgttaaaacatatttattaaattatattattcaacatggttcaattttaggtgaatcacaacaagaattaaattcaatggttacagatacattaaataattcaattccatttaaattatcatcatatacagatgataaaattttaatttcatattttaataaattttttaaaagaattaaatcatcatcagttttaaatatgagatataaaaatgataaatatgttgatacatcaggttatgattcaaatattaatattaatggtgatgtttataaatatccaacaaataaaaatcaatttggtatttataatgataaattatcagaagttaatatttcacaaaatgattatattatttatgataataaatataaaaatttttcaatttcattttgggttagaattccaaattatgataataaaattgttaatgttaataatgaatatacaattattaattgtatgagagataataattcaggttggaaagtttcattaaatcataatgaaattatttggacattacaagataatgcaggtattaatcaaaaattagcatttaattatggtaatgcaaatggtatttcagattatattaataaatggatttttgttacaattacaaatgatagattaggtgattcaaaattatatattaatggtaatttaattgatcaaaaatcaattttaaatttaggtaatattcatgtttcagataatattttatttaaaattgttaattgttcatatacaagatatattggtattagatattttaatatttttgataaagaattagatgaaacagaaattcaaacattatattcaaatgaaccaaatacaaatattttaaaagatttttggggtaattatttattatatgataaagaatattatttattaaatgttttaaaaccaaataattttattgatagaagaaaagattcaacattatcaattaataatattagatcaacaattttattagcaaatagattatattcaggtattaaagttaaaattcaaagagttaataattcatcaacaaatgataatttagttagaaaaaatgatcaagtttatattaattttgttgcatcaaaaacacatttatttccattatatgcagatacagcaacaacaaataaagaaaaaacaattaaaatttcatcatcaggtaatagatttaatcaagttgttgttatgaattcagttggtaataattgtacaatgaattttaaaaataataatggtaataatattggtttattaggttttaaagcagatacagttgttgcatcaacatggtattatacacatatgagagatcatacaaattcaaatggttgtttttggaattttatttcagaagaacatggttggcaagaaaaataa

SEQ ID NO: 49 - DNA, 3759 bp, Artificial Sequence
  mat_peptide (1)...(3756): BoNT/E, D. discoideum-modified 3
atgcctaaaattaattcattcaattataatgatccagttaatgatagaacaattttatatattaaaccaggaggttgtcaagaattttataaaagttttaatattatgaaaaatatatggattataccagaaagaaatgttattggtactacaccacaagatttccatccacctacttcacttaaaaatggtgattcatcttattatgatccaaattatttgcaatcagatgaagaaaaagatagatttttaaaaattgttacaaaaattttcaatcgtattaataataatttatcaggtggaatattattggaagaattatcaaaagcaaatccatatttaggtaatgataatacaccagataatcaatttcatattggtgatgcatcagcagtagaaattaaattcagtaatggttctcaagatatattacttccaaatgttataattatgggtgcagaaccagatttgttcgaaacaaattcatctaatatatcattaagaaataattatatgccatcaaatcatggttttggtagtattgctattgttacattctcaccagaatattcttttagattcaatgataattcaatgaatgaatttattcaagatccagctttgactttaatgcatgaattaattcattcattacatggtttatatggtgctaaaggaattactacaaaatatactattacccaaaaacaaaatccacttattacaaatattcgtggtaccaatattgaagaatttttaacttttggaggtactgatttaaatattataacatcagcacaatctaatgatatttatactaatttattggcagattataaaaaaattgcatcaaaattgagtaaagtacaagtttcaaatccattacttaatccatataaagatgtttttgaagcaaaatatggtttagataaagatgcatcaggaatttatagtgtaaatattaataaatttaatgatatttttaaaaaattgtatagttttaccgaatttgatcttgctactaaatttcaagttaaatgtagacaaacttatattggtcaatataaatattttaaactttcaaatttattgaatgattcaatttataatatttctgaaggatataatattaataatttaaaagttaattttagaggtcaaaatgcaaatttgaatcctcgtattataactcctattactggtcgtggtttagtaaaaaaaataattagattttgtaaaaatattgtttcagtaaaaggtataagaaaatcaatttgtatagaaataaataatggtgaattatttttcgttgcaagtgaaaattcttataatgatgataatattaatacaccaaaagaaattgatgatactgtaactagtaataataattatgaaaatgatttagatcaagttattttaaattttaatagtgaatcagcaccaggtttatcagatgaaaaacttaatttaacaattcaaaatgatgcttatataccaaaatatgattctaatggtacatcagatattgaacaacatgatgttaatgaattaaatgttttcttttatttagatgcacaaaaagtaccagaaggtgaaaataatgttaatttgacatcaagtattgatacagcacttttggaacaaccaaaaatttatacttttttctcaagtgaatttataaataatgttaataaaccagttcaagctgcattattcgtttcatggatacaacaagtattagttgattttacaactgaagctaatcaaaaatcaaccgttgataaaattgctgatatttcaattgttgtaccatatattggattagcattgaatataggtaatgaagcacaaaaaggtaattttaaagatgcattagaattattgggtgcaggtattttacttgaattcgaaccagaattattgattccaactattttagtatttaccattaaaagtttcttaggttcatctgataataaaaataaagttattaaagcaattaataatgctttaaaagaacgtgatgaaaaatggaaagaagtttattctttcattgtttcaaattggatgactaaaattaatactcaatttaataaaagaaaagaacaaatgtatcaagctttacaaaatcaagtaaatgcaattaaaacaataattgaatcaaaatataattcatatacattagaagaaaaaaatgaattaacaaataaatatgatattaaacaaattgaaaatgaattaaatcaaaaagtatcaattgcaatgaataatattgatagattcttaaccgaaagttcaatatcatatttaatgaaattaataaatgaagttaaaataaataaattaagagaatatgatgaaaatgttaaaacttatttacttaattatattatacaacatggttctattttaggtgaatcacaacaagaattaaattcaatggttactgatactttaaataatagtattccatttaaattatcatcttatacagatgataaaattttaatttcatatttcaataaattctttaaaagaattaaatcaagttcagtattaaatatgcgttataaaaatgataaatatgtagatacctcaggttatgattcaaatattaatattaatggtgatgtatataaatatccaacaaataaaaatcaatttggtatttataatgataaattaagtgaagttaatatatcacaaaatgattatattatatatgataataaatataaaaatttttcaatttcattttgggttagaattccaaattatgataataaaattgttaatgttaataatgaatatacaattataaattgtatgagagataataattctggttggaaagtttcattaaatcataatgaaattatatggactttacaagataatgcaggtataaatcaaaaattagcttttaattatggtaatgctaatggtatttcagattatataaataaatggatttttgttacaattacaaatgatagattaggagatagtaaattatatattaatggtaatttaattgatcaaaaaagtatattaaatttaggtaatattcatgtatcagataatatattatttaaaattgttaattgtagttatactagatatattggtattagatattttaatatttttgataaagaattagatgaaacagaaattcaaacattatatagtaatgaaccaaataccaatattttaaaagatttctggggtaattatcttttatatgataaagaatattatttacttaatgtattaaaaccaaataatttcattgatagacgtaaagattctacattatcaataaataatattagatcaacaattttattagcaaatcgtttatatagtggtattaaagttaaaattcaaagagtaaataattcatctaccaatgataatttagttagaaaaaatgatcaagtatatattaattttgttgcttcaaaaactcatttatttcctttatatgcagatacagctacaaccaataaagaaaaaacaattaaaatttcttcaagtggtaatagatttaatcaagttgttgttatgaattcagttggtaataattgtacaatgaattttaaaaataataatggtaataatattggattattaggtttcaaagcagatacagttgttgcatcaacatggtattatacacatatgagagatcatacaaattcaaatggatgtttttggaattttatttcagaagaacatggttggcaagaaaaataa

SEQ ID NO: 50 - DNA, 3759 bp, Artificial Sequence
  mat_peptide (1)...(3756): BoNT/E, Z. mays-modified 1
atgccgaagataaactcatttaactacaacgatcccgtgaatgacaggaccatcctgtacatcaagccgggtggctgtcaggaattctacaaatctttcaacatcatgaaaaacatatggatcattccagaacggaatgtgatcggtactaccccccaggactttcatcctcccacaagccttaagaatggtgacagctcatattacgatccgaactacctgcaatccgatgaggaaaaggaccggtttcttaagatcgtgacgaaaattttcaaccgtatcaacaataacctttctggaggcatcctactggaggaactttctaaggcgaacccatacctcggcaacgacaatacccctgataaccagttccacataggtgatgcatcggcggttgaaattaagtttagcaatgggagccaggacatccttctgccaaacgtcattataatgggggccgagccagacctctttgagactaactcaagcaatatttcgttgcgcaataactacatgccctcaaaccacggctttgggtccatagcaatagtgactttcagccccgaatactccttcagattcaacgataactcaatgaacgaattcattcaggacccggcgctcacgctgatgcacgagctcatccattccctccatggactctacggcgcgaaaggcatcactaccaagtacacaattacacagaagcaaaacccgctcatcacaaacatccgtgggactaatattgaggaatttctgacgttcggcggtaccgatctcaacattatcacgtctgcgcagtctaatgatatctacactaacctgttagcagactacaaaaagattgcctccaagttgtcaaaagtgcaggtatccaaccctctcttaaacccttataaggatgtcttcgaggccaagtatgggttggataaagatgcctctggtatttattccgttaacataaacaagttcaatgatattttcaagaaactttattctttcacagagttcgacttagctaccaagtttcaggtgaagtgtcggcagacttatattggccaatacaaatacttcaaacttagcaacctcctgaacgactcaatttacaacatttccgagggctacaacataaataacctcaaggtgaattttcgcgggcagaatgccaacttgaaccccaggatcataactcctatcaccgggagggggctagtcaaaaagatcattcgcttctgcaagaacattgtatccgtcaaggggatccgaaagagcatctgcatagagatcaataacggcgagttatttttcgtggcgtccgagaattcctataacgatgacaacatcaataccccgaaggagatagacgatacggttacgagcaataacaattatgagaatgatttggaccaggtgatattgaacttcaactcagaatccgctcctggactcagcgatgagaagctcaatctgaccatccaaaacgatgcttacatcccaaagtacgacagtaacggaacatcggatatcgagcagcacgatgtcaacgagcttaatgtttttttctacctcgacgcccagaaagtcccagagggagagaacaatgtgaatctgactagcagtatcgacaccgctctgctcgaacagccgaagatttacacctttttctcgtctgagttcattaacaatgtcaacaagccggtacaagctgcccttttcgtgtcatggattcagcaagtgctggtggacttcaccacggaggcaaaccaaaagtcaactgttgacaagatcgcagacatatccatcgttgtcccttatatcggtcttgccctgaacatcggcaacgaggctcagaagggaaacttcaaggacgcgctagagctgcttggagccggcatactgctcgaattcgaacccgagctactcatcccgaccatcctcgtgtttaccattaagtcattcctcggcagctccgacaacaaaaacaaagtcatcaaagctatcaacaatgcgttgaaggagcgcgacgagaagtggaaggaagtgtacagcttcatcgtttcgaattggatgacgaaaatcaatacccagttcaacaagagaaaggaacaaatgtaccaggccctgcagaatcaagtaaacgcgattaagacgatcattgaatctaaatacaactcttacacgttggaggaaaaaaacgagctaactaataaatacgacatcaagcaaattgaaaatgagttgaaccaaaaggtttctatcgctatgaacaatatcgatcggtttctgacggagtcgtccattagctatcttatgaagctcataaacgaggtcaagataaataaactgagggagtatgacgagaatgtaaagacatatctgcttaattacatcattcagcacggctcaatcttgggggagagtcagcaagagctgaattcgatggtcaccgacaccctaaacaatagcattcccttcaaactttctagctacacagatgacaagatactgatctcatatttcaacaagttctttaaaagaatcaaaagctcctctgtcttgaacatgcggtacaagaatgacaagtacgtcgacacttcggggtacgacagtaacattaacatcaacggtgacgtttataagtatcctacaaacaagaaccagtttgggatttacaacgacaagctctcggaagtgaatatatcgcaaaatgattacataatctacgataacaagtataaaaacttttccattagcttttgggtgcgtatcccaaactacgataataagatagtgaacgtcaacaatgagtacacaattataaactgcatgagggacaacaattctggatggaaggtaagtctaaatcacaacgagattatctggaccctgcaggacaatgcgggcatcaaccagaaattggccttcaactacggaaacgcaaacggcattagcgactacattaataagtggattttcgtcaccattacaaatgatagactgggcgatagtaagctgtatatcaatggcaacctgattgatcaaaagtccattttgaatctcggtaacattcatgtttctgacaacatcttgttcaagatcgttaattgctcatacacgcgctacatcggaataagatactttaatatctttgacaaggagctcgatgagacagaaatccaaactctctattcgaacgaaccaaatacaaatatcttgaaggatttctggggcaactacttactttacgacaaggagtactatctgctaaacgtgttaaagccaaacaatttcatcgaccgtaggaaggacagcactctctctatcaacaatatcaggagtacaatccttctcgccaacaggctctactccggcattaaggtgaagatccagcgcgttaataactcgagtaccaacgataatctcgtccgcaagaacgaccaagtctatatcaacttcgtggccagcaaaacccatcttttcccgctgtacgcagacaccgccacaacgaataaggagaagacgataaagatttcctcaagtggtaatcgattcaaccaggttgtggtcatgaatagcgtcggaaacaattgcactatgaattttaagaataacaatggcaacaacatcggtcttctgggcttcaaagctgataccgtggttgctagtacttggtattatacgcacatgcgcgaccataccaactcgaacggctgtttctggaactttatctccgaggaacacggttggcaggagaagtaa

SEQ ID NO: 51 - DNA, 3759 bp, Artificial Sequence
  mat_peptide (1)...(3756): BoNT/E, Z. mays-modified 2
atgccgaagatcaacagcttcaactacaacgacccggtgaacgacaggaccatcctgtacatcaagccgggcggctgccaggagttctacaagagcttcaacatcatgaagaacatctggatcatcccggagaggaacgtgatcggcaccaccccgcaggacttccacccgccgaccagcctgaagaacggcgacagcagctactacgacccgaactacctgcagagcgacgaggagaaggacaggttcctgaagatcgtgaccaagatcttcaacaggatcaacaacaacctgagcggcggcatcctgctggaggagctgagcaaggccaacccgtacctgggcaacgacaacaccccggacaaccagttccacatcggcgacgccagcgccgtggagatcaagttcagcaacggcagccaggacatcctgctgccgaacgtgatcatcatgggcgccgagccggacctgttcgagaccaacagcagcaacatcagcctgaggaacaactacatgccgagcaaccacggcttcggcagcatcgccatcgtgaccttcagcccggagtacagcttcaggttcaacgacaacagcatgaacgagttcatccaggacccggccctgaccctgatgcacgagctgatccacagcctgcacggcctgtacggcgccaagggcatcaccaccaagtacaccatcacccagaagcagaacccgctgatcaccaacatcaggggcaccaacatcgaggagttcctgaccttcggcggcaccgacctgaacatcatcaccagcgcccagagcaacgacatctacaccaacctgctggccgactacaagaagatcgccagcaagctgagcaaggtgcaggtgagcaacccgctgctgaacccgtacaaggacgtgttcgaggccaagtacggcctggacaaggacgccagcggcatctacagcgtgaacatcaacaagttcaacgacatcttcaagaagctgtacagcttcaccgagttcgacctggccaccaagttccaggtgaagtgcaggcagacctacatcggccagtacaagtacttcaagctgagcaacctgctgaacgacagcatctacaacatcagcgagggctacaacatcaacaacctgaaggtgaacttcaggggccagaacgccaacctgaacccgaggatcatcaccccgatcaccggcaggggcctggtgaagaagatcatcaggttctgcaagaacatcgtgagcgtgaagggcatcaggaagagcatctgcatcgagatcaacaacggcgagctgttcttcgtggccagcgagaacagctacaacgacgacaacatcaacaccccgaaggagatcgacgacaccgtgaccagcaacaacaactacgagaacgacctggaccaggtgatcctgaacttcaacagcgagagcgccccgggcctgagcgacgagaagctgaacctgaccatccagaacgacgcctacatcccgaagtacgacagcaacggcaccagcgacatcgagcagcacgacgtgaacgagctgaacgtgttcttctacctggacgcccagaaggtgccggagggcgagaacaacgtgaacctgaccagcagcatcgacaccgccctgctggagcagccgaagatctacaccttcttcagcagcgagttcatcaacaacgtgaacaagccggtgcaggccgccctgttcgtgagctggatccagcaggtgctggtggacttcaccaccgaggccaaccagaagagcaccgtggacaagatcgccgacatcagcatcgtggtgccgtacatcggcctggccctgaacatcggcaacgaggcccagaagggcaacttcaaggacgccctggagctgctgggcgccggcatcctgctggagttcgagccggagctgctgatcccgaccatcctggtgttcaccatcaagagcttcctgggcagcagcgacaacaagaacaaggtgatcaaggccatcaacaacgccctgaaggagagggacgagaagtggaaggaggtgtacagcttcatcgtgagcaactggatgaccaagatcaacacccagttcaacaagaggaaggagcagatgtaccaggccctgcagaaccaggtgaacgccatcaagaccatcatcgagagcaagtacaacagctacaccctggaggagaagaacgagctgaccaacaagtacgacatcaagcagatcgagaacgagctgaaccagaaggtgagcatcgccatgaacaacatcgacaggttcctgaccgagagcagcatcagctacctgatgaagctgatcaacgaggtgaagatcaacaagctgagggagtacgacgagaacgtgaagacctacctgctgaactacatcatccagcacggcagcatcctgggcgagagccagcaggagctgaacagcatggtgaccgacaccctgaacaacagcatcccgttcaagctgagcagctacaccgacgacaagatcctgatcagctacttcaacaagttcttcaagaggatcaagagcagcagcgtgctgaacatgaggtacaagaacgacaagtacgtggacaccagcggctacgacagcaacatcaacatcaacggcgacgtgtacaagtacccgaccaacaagaaccagttcggcatctacaacgacaagctgagcgaggtgaacatcagccagaacgactacatcatctacgacaacaagtacaagaacttcagcatcagcttctgggtgaggatcccgaactacgacaacaagatcgtgaacgtgaacaacgagtacaccatcatcaactgcatgagggacaacaacagcggctggaaggtgagcctgaaccacaacgagatcatctggaccctgcaggacaacgccggcatcaaccagaagctggccttcaactacggcaacgccaacggcatcagcgactacatcaacaagtggatcttcgtgaccatcaccaacgacaggctgggcgacagcaagctgtacatcaacggcaacctgatcgaccagaagagcatcctgaacctgggcaacatccacgtgagcgacaacatcctgttcaagatcgtgaactgcagctacaccaggtacatcggcatcaggtacttcaacatcttcgacaaggagctggacgagaccgagatccagaccctgtacagcaacgagccgaacaccaacatcctgaaggacttctggggcaactacctgctgtacgacaaggagtactacctgctgaacgtgctgaagccgaacaacttcatcgacaggaggaaggacagcaccctgagcatcaacaacatcaggagcaccatcctgctggccaacaggctgtacagcggcatcaaggtgaagatccagagggtgaacaacagcagcaccaacgacaacctggtgaggaagaacgaccaggtgtacatcaacttcgtggccagcaagacccacctgttcccgctgtacgccgacaccgccaccaccaacaaggagaagaccatcaagatcagcagcagcggcaacaggttcaaccaggtggtggtgatgaacagcgtgggcaacaactgcaccatgaacttcaagaacaacaacggcaacaacatcggcctgctgggcttcaaggccgacaccgtggtggccagcacctggtactacacccacatgagggaccacaccaacagcaacggctgcttctggaacttcatcagcgaggagcacggctggcaggagaagtaa

SEQ ID NO: 52 - DNA, 3759 bp, Artificial Sequence
  mat_peptide (1)...(3756): BoNT/E, Z. mays-modified 3
atgccaaagattaattcatttaattacaacgatccggtcaacgacaggaccatcctctacatcaagcccggcggatgccaggaattctacaagtcattcaacatcatgaagaatatttggataattcccgagcgaaacgtaattggaactaccccgcaggactttcacccgccaacgtcgttaaaaaacggcgactcaagttactatgatcccaactacctccaaagtgatgaggaaaaagacagattcttaaaaatcgttacgaaaatattcaaccgcataaacaataacctgtctgggggaatcctcttggaagagctatctaaggcaaacccttatctgggcaacgataataccccagacaaccaattccatatcggcgacgcgtctgcagtcgagattaaattctccaacggaagccaggatatcctgctccccaatgttattatcatgggcgccgaaccagacctctttgagactaatagttctaatatatcccttcggaacaattatatgccatcgaaccacggtttcggctctattgctattgttacgttcagtccggagtattccttccggtttaatgataatagtatgaatgagttcattcaggacccagcacttacacttatgcatgaattaatccattctctgcacgggctttatggcgcaaagggaattaccacgaagtacactatcacccaaaagcaaaatccactgatcacgaatatcagaggaaccaacattgaagagttcctcacttttggcggtacggacctgaacattatcacatcggcccagtcaaacgacatttataccaacctcctggccgactacaaaaagatcgcttccaagctcagcaaagtccaggtttctaatcccttacttaatccgtataaggacgtgtttgaggccaagtatggtctggacaaagatgcaagtgggatatactctgtgaacatcaacaagtttaacgacatttttaaaaagctctactccttcacagagtttgacctcgctaccaagttccaggtgaagtgtcgtcagacgtacattgggcaatacaagtactttaagctcagcaacctactgaacgactcaatctacaatatatctgaaggctataatattaacaatctgaaggtcaactttcgcgggcagaatgccaacctcaatcctcgtattataacccctatcacaggccgcggcttggtgaaaaagatcattaggttttgtaaaaacatcgtttcggttaagggtataaggaagtccatatgcatcgagataaataacggagagctattctttgtggcttcagaaaactcgtacaacgacgataatatcaacaccccgaaggagatcgatgacacggtgacttctaacaataactacgaaaatgacctcgatcaagtcatcctcaacttcaattccgagagcgccccgggtcttagcgacgagaagctaaacctcaccatacagaatgacgcctatattcccaagtatgacagtaacgggactagcgacatagagcagcatgacgtcaacgagctaaatgtattcttttatctagacgcgcagaaggtgcctgaaggcgaaaacaatgtcaaccttacctcgtctatcgacactgccctcctggagcaaccgaagatctacacattcttttcatccgagttcatcaataacgtcaacaagcccgtccaggccgctctgttcgtctcctggattcagcaagtcctggttgatttcactacggaggcgaatcagaaatcaaccgttgacaagatcgccgacatttctattgtcgtgccatacatcggcctagcgctcaatattggaaacgaggcacaaaaaggcaacttcaaggacgctcttgagctgcttggtgcgggtatccttctcgaattcgagcccgagcttctgataccaactatcctggtctttaccattaagagctttctcggttcatccgataataaaaacaaggttatcaaggctattaacaatgcgttgaaggagcgcgacgaaaaatggaaggaggtgtactccttcatcgtttcaaactggatgactaagatcaatactcagttcaacaaaagaaaggaacaaatgtatcaggccctccaaaaccaagtcaacgctataaagaccatcatagagtcgaagtacaatagctacactctggaggaaaaaaacgagcttacgaacaagtacgacatcaaacagattgagaacgaactcaatcagaaggtctcgatcgccatgaacaatatcgacaggtttcttacagaatcctcgatttcctatctcatgaagctcatcaatgaagttaagatcaacaagctgcgggagtacgatgagaatgtgaagacatacctgttgaactacatcatacagcatggctcgatcttgggcgaatcgcagcaagaactgaacagtatggtgaccgataccctaaataactcaataccttttaagttgagttcttatacggatgacaagatcctcataagttatttcaacaagttctttaagcgtattaagtcttcaagcgtactcaacatgcgatacaaaaatgataagtacgtggacacatctggttacgatagcaatatcaacataaacggcgatgtgtacaaataccctacaaataagaaccagttcggcatttataatgataagctttccgaggtgaacatttcacaaaatgattacatcatttacgataacaagtacaagaacttctccatcagcttctgggtcaggatcccgaactacgataataagattgtgaacgtaaacaatgagtacaccatcattaactgcatgcgggataacaatagcggctggaaggtgtcattgaatcacaacgagataatttggaccctccaagataacgccggcatcaatcagaagctggcgttcaattatggaaacgctaatggcatctcagactacatcaacaaatggatattcgttacaatcactaacgatcgcctgggggatagcaagctttatattaacgggaacttgattgatcagaagtccattttgaacctgggcaacatccacgtcagcgacaacatcctgttcaagatcgtgaactgtagctacacccgctacatcggtatccgttacttcaatattttcgataaggagctcgacgagacggagatccagactctgtactcgaacgagccgaacacaaacatcttaaaagacttctgggggaactacttgctttatgataaagagtattacctgttgaacgtgttgaagcctaataacttcattgaccgcaggaaggactcgacattatccattaacaatattaggagcaccatcctgttggcgaatagactctactccgggatcaaggtgaagatccagcgggtaaacaattccagcaccaacgacaacttggtcagaaagaacgaccaggtgtacatcaacttcgtggcgagcaaaacacatttgttccctctgtacgccgataccgcaacaacgaacaaggagaagacaatcaaaataagctccagcggcaacaggttcaaccaagtcgtggtaatgaattccgtgggaaacaattgcacgatgaatttcaagaacaataacgggaacaatatagggcttcttggttttaaagcggatacggttgttgcttctacctggtactatactcacatgcgcgaccacactaacagcaacggttgcttttggaatttcatcagcgaggaacacggatggcaggagaaataa

SEQ ID NO: 53 - DNA, 3759 bp, Artificial Sequence
  mat_peptide (1)...(3756): BoNT/E, A. thaliana-modified 1
atgcccaaaatcaactcttttaattataacgatcctgttaacgatcgcacgattttatacatcaagccaggaggttgtcaagagttctataaaagttttaatattatgaaaaatatatggattataccggagcgtaacgttatcggaacgactcctcaggacttccatccaccgacaagtctgaaaaatggtgattccagttactatgatcccaattatctacaatctgacgaggaaaaagaccgttttctaaagatcgtcaccaaaatttttaacaggatcaataacaatttaagcggcgggatcctgctcgaggaattatcgaaagctaacccttatctgggcaacgataatactccggataatcaatttcatatcggagatgcatcagccgtcgagatcaaattctcgaatggttctcaagatatactcttgccaaacgttatcataatgggcgcagagccagacttatttgagacaaattcctcgaatatttccctccgcaacaattatatgccatcgaaccatggttttggttcgatagctattgtgacattctccccagaatattcattcagatttaacgataactcgatgaacgagtttatacaagaccctgctcttacacttatgcatgaactcattcacagcctgcatggactttatggtgctaaggggattactacaaagtacacgattactcaaaaacagaatcctttgataacaaatattcggggtacaaacatcgaagagttcttaacattcggaggcactgatctaaacataatcacatcagcgcaaagtaatgatatctatactaacctccttgcagattacaaaaagatagcttctaaactatcaaaggtgcaagtgtcgaacccattgctgaatccttacaaagacgttttcgaagctaaatatggattggataaagatgctagcggaatatacagtgtgaacattaataagttcaatgacatctttaagaaactatatagtttcacggagtttgacctagcgacaaagttccaagtgaaatgccgtcagacctacataggtcagtacaagtacttcaagctgtctaacctgttaaatgactctatttacaacatctctgaaggatacaatattaataacttaaaggttaattttcggggacagaacgctaaccttaacccaaggattataaccccaataacgggaaggggtctcgttaaaaagatcattagattctgcaaaaacattgtgtcggttaaaggtattaggaagagcatttgtatagagattaataacggagaactgtttttcgttgcttcagagaattcttataatgatgacaatatcaatactcccaaggaaatcgatgacactgtcacaagcaataacaattacgagaatgatctggaccaagttattttgaactttaacagcgaatccgctccgggtttgtccgatgaaaagctaaatttaactattcaaaacgacgcatacatccctaaatatgatagtaacggcactagcgatatcgagcaacatgatgttaacgagctcaatgtttttttctatcttgatgctcaaaaggtccctgaaggagagaacaatgtaaatttaactagctccatcgacacggcgcttctagagcagccaaagatatatactttctttagcagtgagttcatcaacaatgtgaacaaaccagttcaagctgcactttttgtttcatggattcagcaagtcctagtggactttaccacagaagctaaccaaaagagtactgtagacaagattgccgacatatcgatcgttgtcccatacataggtctcgctttgaatattggaaatgaagcgcagaaaggcaacttcaaggatgcacttgagctcctaggggcaggaatcctgttggaatttgaacctgagttactgattccgacaatattagtgttcactatcaagagttttctcggaagctctgataataagaacaaagtgatcaaagcgatcaacaatgctctcaaggaaagagatgagaaatggaaagaggtttattcatttatcgtctcaaactggatgaccaaaattaatacacaatttaacaagcgtaaggagcaaatgtatcaggccctccaaaatcaagtaaatgcaatcaagaccatcattgaaagcaagtataactcgtacacccttgaagagaagaacgagctgacgaacaaatatgatattaagcaaattgaaaatgaattaaaccagaaggtttccattgcaatgaacaatatcgaccggttcctaaccgagtcatccatttcttatctaatgaaactcataaacgaagtgaaaatcaacaagttgcgagaatatgacgaaaacgtaaaaacttaccttctaaactatatcattcagcatggttccatacttggagaatcccagcaagaattgaactcaatggttacagacactctgaacaattcaataccttttaagttgagttcatacactgatgacaagatattaattagctattttaataaatttttcaaaagaatcaagtcttcatctgtactaaatatgagatataaaaatgataaatacgtcgacacctcgggctatgatagtaacattaatattaacggtgatgtatacaagtatcctaccaacaagaatcagttcggaatttataatgataagctctctgaggttaatatctcccagaatgactacattatctacgataacaaatacaagaacttctccataagcttctgggttagaatacctaattacgataacaagatcgtcaatgtgaataacgagtacactattatcaattgtatgagagataataactccggttggaaagtgtctttaaaccacaatgaaataatttggacgctacaggataatgcaggaatcaaccagaagctggcatttaactacgggaacgccaatggcattagtgattacattaataagtggatatttgttacgattacaaacgatcgtctgggggatagtaaactttatataaacggtaatttgattgatcaaaagtctattttgaatttgggtaacattcacgtctcagataacattctgttcaaaatcgtcaattgttcatacactcgatatatcggtatcagatacttcaacatctttgataaagagctcgatgaaaccgagatccagacattatattccaatgaaccgaacaccaatattctcaaggatttttggggaaactacttgctttatgacaaggaatactatttgctaaatgttcttaaacctaataacttcatcgatcgaaggaaggattcaacattatccatcaataacattagaagcacaattctacttgctaatcgattatatagtgggataaaggtaaaaattcagagggttaacaattcgagcaccaatgataatttagttaggaaaaatgatcaggtgtacattaactttgtggcctcgaagacccacctgttccccctctatgcggatactgctactacaaacaaagagaaaactataaaaataagttcgtctgggaatagattcaatcaagtcgtggtaatgaattcggtagggaataactgcacaatgaactttaaaaacaataacggaaataatattgggttgcttggtttcaaggctgatacggttgttgccagcacttggtactatacgcacatgagagatcatactaatagtaatggttgtttctggaattttatatcggaagaacatggatggcaagaaaagtaa

SEQ ID NO: 54 - DNA, 3759 bp, Artificial Sequence
  CDS (1)...(3759): BoNT/E, A. thaliana-modified 2
atgcctaagattaatagttttaattataatgatcctgttaatgatagaactattttatatattaagcctggaggatgtcaagaattttataagagttttaatattatgaagaatatttggattattcctgaaagaaatgttattggaactactcctcaagattttcatcctcctactagtttaaagaatggagatagtagttattatgatcctaattatttacaaagtgatgaagaaaaggatagatttttaaagattgttactaagatttttaatagaattaataataatttaagtggaggaattttattagaagaattaagtaaggctaatccttatttaggaaatgataatactcctgataatcaatttcatattggagatgctagtgctgttgaaattaagtttagtaatggaagtcaagatattttattacctaatgttattattatgggagctgaacctgatttatttgaaactaatagtagtaatattagtttaagaaataattatatgcctagtaatcatggatttggaagtattgctattgttacttttagtcctgaatatagttttagatttaatgataatagtatgaatgaatttattcaagatcctgctttaactttaatgcatgaattaattcatagtttacatggattatatggagctaagggaattactactaagtatactattactcaaaagcaaaatcctttaattactaatattagaggaactaatattgaagaatttttaacttttggaggaactgatttaaatattattactagtgctcaaagtaatgatatttatactaatttattagctgattataagaagattgctagtaagttaagtaaggttcaagttagtaatcctttattaaatccttataaggatgtttttgaagctaagtatggattagataaggatgctagtggaatttatagtgttaatattaataagtttaatgatatttttaagaagttatatagttttactgaatttgatttagctactaagtttcaagttaagtgtagacaaacttatattggacaatataagtattttaagttaagtaatttattaaatgatagtatttataatattagtgaaggatataatattaataatttaaaggttaattttagaggacaaaatgctaatttaaatcctagaattattactcctattactggaagaggattagttaagaagattattagattttgtaagaatattgttagtgttaagggaattagaaagagtatttgtattgaaattaataatggagaattattttttgttgctagtgaaaatagttataatgatgataatattaatactcctaaggaaattgatgatactgttactagtaataataattatgaaaatgatttagatcaagttattttaaattttaatagtgaaagtgctcctggattaagtgatgaaaagttaaatttaactattcaaaatgatgcttatattcctaagtatgatagtaatggaactagtgatattgaacaacatgatgttaatgaattaaatgtttttttttatttagatgctcaaaaggttcctgaaggagaaaataatgttaatttaactagtagtattgatactgctttattagaacaacctaagatttatactttttttagtagtgaatttattaataatgttaataagcctgttcaagctgctttatttgttagttggattcaacaagttttagttgattttactactgaagctaatcaaaagagtactgttgataagattgctgatattagtattgttgttccttatattggattagctttaaatattggaaatgaagctcaaaagggaaattttaaggatgctttagaattattaggagctggaattttattagaatttgaacctgaattattaattcctactattttagtttttactattaagagttttttaggaagtagtgataataagaataaggttattaaggctattaataatgctttaaaggaaagagatgaaaagtggaaggaagtttatagttttattgttagtaattggatgactaagattaatactcaatttaataagagaaaggaacaaatgtatcaagctttacaaaatcaagttaatgctattaagactattattgaaagtaagtataatagttatactttagaagaaaagaatgaattaactaataagtatgatattaagcaaattgaaaatgaattaaatcaaaaggttagtattgctatgaataatattgatagatttttaactgaaagtagtattagttatttaatgaagttaattaatgaagttaagattaataagttaagagaatatgatgaaaatgttaagacttatttattaaattatattattcaacatggaagtattttaggagaaagtcaacaagaattaaatagtatggttactgatactttaaataatagtattccttttaagttaagtagttatactgatgataagattttaattagttattttaataagttttttaagagaattaagagtagtagtgttttaaatatgagatataagaatgataagtatgttgatactagtggatatgatagtaatattaatattaatggagatgtttataagtatcctactaataagaatcaatttggaatttataatgataagttaagtgaagttaatattagtcaaaatgattatattatttatgataataagtataagaattttagtattagtttttgggttagaattcctaattatgataataagattgttaatgttaataatgaatatactattattaattgtatgagagataataatagtggatggaaggttagtttaaatcataatgaaattatttggactttacaagataatgctggaattaatcaaaagttagcttttaattatggaaatgctaatggaattagtgattatattaataagtggatttttgttactattactaatgatagattaggagatagtaagttatatattaatggaaatttaattgatcaaaagagtattttaaatttaggaaatattcatgttagtgataatattttatttaagattgttaattgtagttatactagatatattggaattagatattttaatatttttgataaggaattagatgaaactgaaattcaaactttatatagtaatgaacctaatactaatattttaaaggatttttggggaaattatttattatatgataaggaatattatttattaaatgttttaaagcctaataattttattgatagaagaaaggatagtactttaagtattaataatattagaagtactattttattagctaatagattatatagtggaattaaggttaagattcaaagagttaataatagtagtactaatgataatttagttagaaagaatgatcaagtttatattaattttgttgctagtaagactcatttatttcctttatatgctgatactgctactactaataaggaaaagactattaagattagtagtagtggaaatagatttaatcaagttgttgttatgaatagtgttggaaataattgtactatgaattttaagaataataatggaaataatattggattattaggatttaaggctgatactgttgttgctagtacttggtattatactcatatgagagatcatactaatagtaatggatgtttttggaattttattagtgaagaacatggatggcaagaaaagtaa

SEQ ID NO: 55 - DNA, 3759 bp, Artificial Sequence
  mat_peptide (1)...(3756): BoNT/E, A. thaliana-modified 3
atgccgaagatcaacagttttaattataacgatcctgtgaacgaccgtacaattttatacataaagccgggcggttgccaggaattctacaagagtttcaacattatgaaaaacatttggataattccggaaagaaatgtgattggtactacacctcaggactttcacccaccgactagtttaaaaaacggggatagtagttactatgatcctaattacttacaaagtgatgaggaaaaggacaggttcttaaagattgttacaaagatattcaaccgtatcaataacaatttaagtggagggatattattagaggaattaagtaaagcgaatccatatttaggcaatgataatactccagacaaccagtttcatatcggagacgctagtgctgttgagattaaatttagtaacggtagtcaagacatattattaccaaacgttattataatgggagctgaacctgatttattcgagaccaacagtagtaacataagtttacgaaataactatatgcctagtaatcatggtttcggaagtattgccattgtcacctttagtcctgagtatagttttaggtttaatgataatagtatgaatgaattcatccaggacccagctttaacattaatgcatgagttaatccatagtttacacggattatacggagcgaaaggtatcactacaaagtatactattacacagaaacaaaacccattaattaccaacatcagagggacaaacattgaggaatttttaacgttcggaggcacggatttaaatattatcaccagtgctcagagtaatgacatttataccaatttattagcggattataagaaaattgccagtaaattaagtaaagttcaagtgagtaacccattattaaacccttacaaagacgtgttcgaggcgaagtacggattagacaaggatgctagtgggatatacagtgttaatattaacaaattcaacgatattttcaagaaattatatagttttactgagttcgatttagctactaagtttcaggttaagtgtagacagacctacataggtcagtataagtattttaagttaagtaacttattaaatgatagtatttataacatcagtgagggttataatattaacaatttaaaggtaaatttccggggacagaacgctaatttaaacccaagaattatcactcctatcactgggagaggattagtcaagaaaattatcagattttgcaaaaacattgtaagtgttaagggaattcgaaagagtatttgtatcgaaattaataacggtgaattatttttcgtcgcaagtgagaacagttataacgatgacaatattaacacgccgaaggagatagacgataccgttacaagtaataacaattatgaaaacgacttagatcaggtgatattaaacttcaatagtgagagtgctccaggattaagtgatgagaaattaaatttaacgatccagaatgacgcctacattccaaaatatgatagtaatgggacgagtgatatcgaacaacatgatgttaatgaattaaacgtttttttctatttagatgcacaaaaagtgcctgaaggagaaaataacgtcaatttaactagtagtatagatacagcattattagagcagcctaaaatatacactttctttagtagtgagttcattaacaatgttaataaacctgtacaagcagctttatttgtgagttggatccaacaggttttagttgatttcactacggaagcgaatcaaaaaagtaccgtggataaaatagctgatattagtatagtagtgccttacattgggttagccttaaatattggtaacgaagcacaaaagggaaacttcaaagatgctttagagttattaggagctggtatattattagaattcgagcctgaattattaatacctaccatattagtttttactataaaaagttttttagggagtagtgataacaaaaataaagtgattaaggcaataaataacgcattaaaggaacgtgatgaaaaatggaaagaggtgtatagttttatcgtcagtaactggatgacgaagattaatacacaatttaacaagaggaaagagcaaatgtatcaagccttacagaatcaggtcaatgctattaagactattatcgaaagtaaatacaacagttacacattagaggaaaaaaacgagttaacaaacaagtacgatatcaaacaaattgaaaatgaattaaatcaaaaagtcagtatcgcaatgaataacatagatcgattcttaactgagagtagtatcagttatttaatgaaattaattaatgaggtaaagattaataaattacgggagtacgacgaaaatgtcaaaacctacttattaaattacattatacagcatggtagtattttaggcgagagtcaacaggaattaaacagtatggtaacagacacgttaaacaatagtatcccatttaaattaagtagttatacagacgataagatcttaataagttattttaataaatttttcaaacgaattaaaagtagtagtgttttaaacatgcggtacaagaatgacaagtatgtcgatacaagtggttacgatagtaatatcaatatcaacggagatgtatataagtacccaactaataaaaatcaattcggcatatataatgacaaattaagtgaagtgaacattagtcaaaatgactacataatttatgataataaatataagaatttcagtatcagtttttgggtaagaattcctaactacgataacaagatcgtgaatgtaaacaatgaatacaccattataaattgtatgagagataataacagtggttggaaggttagtttaaatcacaacgaaatcatatggacattacaagacaatgctggtatcaatcaaaaattagctttcaactatggtaatgctaatggaattagtgattacataaacaagtggatttttgttacaattacgaacgataggttaggtgatagtaaattatacattaacgggaacttaatcgatcaaaagagtatcttaaatttaggaaatatacacgttagtgataacattttatttaagatcgtcaactgtagttacacgagatacatcggtatcagatattttaacatctttgataaggagttagatgaaacagagatccaaactttatatagtaatgagcctaacacaaatatcttaaaggatttttggggcaattatttattatacgataaggagtactatttattaaatgtgttaaagcctaacaatttcatcgatcgtagaaaagatagtacgttaagtattaacaatatccgtagtaccatcttattagcaaataggttatatagtggaatcaaagtcaagatacaaagagttaacaatagtagtaccaacgataacttagttagaaagaacgaccaagtgtacatcaacttcgttgccagtaagactcacttattcccgttatacgcggatactgcaacaactaataaagaaaagacaatcaagatcagtagtagtggaaataggttcaaccaggttgtggttatgaacagtgttggtaataactgtactatgaacttcaagaacaataacggcaataacataggcttattaggttttaaggcagacaccgttgtcgctagtacttggtattatactcatatgagggaccatactaatagtaatggatgcttttggaactttatcagtgaggaacatggttggcaagaaaagtaa

SEQ ID NO: 56 - DNA, 3759 bp, Artificial Sequence
  mat_peptide (1)...(3756): BoNT/E, D. melanogaster-modified 1
atgcctaagatcaattcttttaactacaacgacccagtgaacgaccgcaccattctatacattaaaccgggcggttgtcaggaattttacaagtcgtttaacatcatgaagaacatctggatcattccggaacgaaatgtgatcggcactacaccacaagacttccatcccccaacgtcacttaagaatggcgattcttcctattacgaccccaattatctgcagtcggacgaagagaaggatcggttccttaagattgtgaccaagatttttaataggatcaataacaatctgagcggcggaattctactggaagagctgagcaaggccaacccctacctcggtaacgacaatactccggacaatcaattccatattggagacgcgtcggccgtggaaattaaattttccaatggttcccaggacatcctgttgcccaacgtgatcattatgggcgcagagcccgatctgtttgaaaccaattcgagcaacatatcgctacggaacaattacatgccgagcaaccatggatttggttcaatcgccatcgttaccttcagccctgagtacagctttcgcttcaacgacaatagcatgaacgagttcatccaggatccggccctcactctgatgcatgagctgattcattcgctgcacggtctctacggagccaagggcataacaacgaagtataccatcacacagaagcagaacccgcttatcacaaatatccgtggaactaacatcgaggaattcctcacattcggcggaaccgatctgaatatcattacgagcgcccagagtaatgatatctacactaacctgcttgcagactataagaaaattgcctcgaagcttagcaaggtgcaagtgtcaaatcctttgttaaacccatacaaagatgtttttgaggccaagtacggactggacaaggatgcaagcggaatctactccgtcaacataaacaaattcaacgatattttcaagaaattatactcctttaccgagtttgatctggccacaaaattccaggtgaagtgcagacagacatatattgggcaatacaaatatttcaagctgagcaatctattgaatgactcgatatacaatatcagcgagggctacaatataaataacttgaaggtgaacttccggggccagaatgctaatctgaacccgaggatcattacgccaatcaccggccgcggtttagtaaagaaaattatacgcttctgcaaaaatatcgtgtcggttaaggggataaggaaatcgatctgcatcgagattaataacggtgagttgttctttgtcgcgtccgaaaattcctacaacgacgataacattaacacgcccaaggaaatcgacgatactgtcaccagtaataacaattatgagaatgatctggatcaggtcatcctgaactttaacagcgaatcagcaccagggcttagcgatgaaaaactcaatctaaccattcaaaatgacgcttacattcctaagtacgattcgaatggcacaagtgacatcgagcaacacgatgtaaacgagctcaacgttttcttttatttagatgcgcagaaagttcccgagggtgagaacaatgtgaacttgacgtcctctatcgataccgctctcttggagcaaccaaagatctacacgttctttagctcggagtttatcaataacgtaaacaagccggttcaagccgcgctgtttgtgagctggatccagcaagtgcttgtcgacttcaccacggaggctaaccagaagagcaccgtggataaaatcgccgacatcagcattgttgtgccctacataggactggccttgaacatcggcaacgaggcgcaaaagggaaatttcaaagacgcgctggagctcttgggagctggaatcttgctcgagttcgaaccagaactgctaattccgaccatcctggtcttcacgattaagtcttttttgggcagtagcgataacaagaacaaggttattaaggcaatcaataacgccctcaaggagagagatgagaaatggaaggaggtctacagcttcattgtctctaactggatgactaaaataaatacacagttcaacaagcgaaaggagcagatgtaccaggcattgcagaaccaggtcaatgccatcaagaccataattgagtcgaagtacaactcctacaccctggaagagaagaatgaactgacaaacaagtatgacatcaagcagatcgagaatgaactgaaccagaaggtaagtatagccatgaacaatatcgatcgtttcctgacggagagtagcattagttatcttatgaagctgattaatgaagtgaaaatcaataagttgcgtgagtacgacgagaacgtcaaaacctacctcctgaattatattatccagcatggcagtattctcggtgagtcgcagcaagagctgaactcgatggtgaccgacacactgaataactccatcccattcaaactgagttcctatacggatgacaagatccttatttcctactttaacaagttctttaagcgaatcaagtccagctcggtcttgaatatgcgctacaagaatgacaagtacgttgacacttcaggctacgactccaatatcaacataaacggagacgtgtataagtatccgaccaacaagaaccaattcggcatatacaacgataagctaagtgaagtaaatatctcacagaacgattatattatatacgataataaatataagaatttctcaataagtttctgggttcgaattcccaactacgataataagatcgtgaatgtgaacaatgaatataccataattaattgtatgcgcgacaacaattccggttggaaggtgtctttgaaccacaatgaaatcatttggaccctgcaggacaacgccgggatcaaccagaaactggctttcaactatggcaacgccaatggcatctcggattacatcaacaagtggattttcgtcacgataacgaacgaccggctgggtgattccaaactgtacattaatggcaacttgatcgatcagaagagtatactgaacctgggcaacattcacgtgtccgacaacattttgttcaagatcgtgaattgctcctacacgaggtatattggcatacgatacttcaacatcttcgacaaagaactggatgagaccgagatacagacgctgtactctaacgagcccaacacgaacattctcaaggacttctggggaaattacctgctctatgataaggagtactatctgttgaatgtgctaaagcccaacaatttcatagatcgtcgcaaggattccaccctgtccatcaacaatatacgcagtaccattctcttagccaatcgcctctattccggcataaaagtcaaaatccagcgtgtgaacaattctagcaccaatgataacctagtacggaaaaatgatcaggtgtacatcaactttgtagcttccaaaacacacctgtttcccctgtatgcggatactgcaactacgaataaggagaaaacgataaagatttcctcgtcaggaaaccgcttcaaccaagtggttgtcatgaatagcgtgggcaataactgcacaatgaacttcaagaacaataacggcaacaatataggcttgcttggtttcaaggcggatacagtcgtggcttcgacttggtattatacccacatgagagaccacactaactcaaacggatgcttttggaactttatctcggaggagcacggatggcaggagaagtaa

SEQ ID NO: 57 - DNA, 3759 bp, Artificial Sequence
  mat_peptide (1)...(3756): BoNT/E, D. melanogaster-modified 2
atgccaaagataaactctttcaactacaatgacccagtgaacgataggacaattttgtatatcaagccgggaggttgtcaggaattttataagtccttcaatattatgaagaacatttggattatacccgaacgaaacgtcatcggcacgaccccacaagacttccacccacccacgagtctcaagaacggtgactccagctactatgatcctaattacttacagtccgatgaggaaaaggataggttcctcaagatagtgacaaaaattttcaaccgaatcaacaataaccttagcggcggaatcctgttggaggaactgtccaaggctaatccctacttaggcaacgataacacaccagacaatcagttccacataggcgacgctagcgccgtggaaatcaagttttccaacggctcgcaggatatcttgctgccgaacgtgatcataatgggtgctgagccggacctgttcgagacaaactcgtctaatattagtcttcgcaataactacatgccctcgaaccatggattcggcagcatcgccatagttaccttctcgcctgagtacagttttcgtttcaatgataattcgatgaatgagttcatacaagatcccgcactgactttgatgcatgagctcatccatagcttacatggcttgtatggtgccaagggtatcacaaccaagtacaccatcacccagaaacaaaatccactcatcacaaatatccgcggaacaaatatcgaggaattcttgaccttcggaggcacagatcttaacataatcacatcagcgcagagtaacgatatttatactaatctgcttgcggattataagaaaatcgcgagtaagctgtcaaaagtccaggtgagtaatccgctgctcaacccgtataaggatgtttttgaagctaagtatggcctggacaaggacgccagcgggatctactcagtgaacattaacaagtttaacgacatttttaagaaactgtacagtttcaccgagtttgacctggccaccaagttccaagttaaatgccggcagacctacataggacaatacaaatacttcaagttgagtaatctgttaaatgattcgatatataacatctccgagggctacaatatcaacaatctcaaggttaactttcgcggacagaatgccaacctgaacccccgcatcattacacccattacaggcaggggcctggtcaaaaagatcatacgattctgcaaaaacatcgtcagcgtgaagggcataagaaagagcatctgcatcgaaatcaacaatggtgagctatttttcgtggccagcgagaatagctacaacgatgacaatattaacaccccgaaagagatagacgataccgtcaccagcaataacaattacgagaatgatctggaccaggtcattttgaatttcaactctgagtcagcccctggcctgtcggatgagaagctcaatctcacgatacagaacgacgcatatatcccgaagtacgactccaatggcacttccgacatcgaacagcatgatgtgaacgaacttaacgtctttttctacctagacgcccagaaggtccccgagggggagaataacgtcaatctcacgagcagtatagatacggctctgttggagcagccgaagatctacacctttttcagcagtgagttcatcaataacgttaataagccggttcaggcggccctgttcgtgtcgtggatccagcaagtgcttgtggatttcacgaccgaagcgaatcaaaagtcgactgtggacaagattgcagacatctcgattgtggtcccatatatcggactggcactaaatatcgggaacgaagcccagaaaggtaattttaaggatgcattggagctactgggagcaggaatactactggagttcgaaccggaactgttgattcccaccatactcgtgttcaccatcaaatcctttctgggttccagcgacaataagaacaaggtgataaaagcgattaacaatgccttgaaggagcgcgacgaaaagtggaaggaggtctatagttttatagtgtcaaattggatgaccaagatcaacacccaatttaacaagcggaaggagcagatgtaccaggcactgcagaaccaagtgaacgctatcaaaaccatcattgagtccaagtacaattcgtacactttggaagagaaaaatgagctgacgaacaaatacgatatcaagcaaatcgagaatgagctgaaccagaaagtgagtattgcgatgaataacatcgaccgtttcctcacggaaagctccatctcgtacctgatgaagcttattaatgaggtgaagattaacaagctgcgcgagtacgacgagaacgtaaagacgtacttgctgaactatattatccaacacggttcaatcctgggagagtcccagcaagagctgaatagcatggtgactgatacactcaacaatagtatcccattcaagctctcatcctacaccgatgacaaaattctaatcagctatttcaataaattctttaaacggattaaaagctcatcggtcctcaatatgcgctacaaaaacgacaagtatgtagatacctccggatatgatagcaacatcaacataaatggcgacgtgtataagtaccccaccaacaagaatcagtttggaatttataacgacaagctgtccgaggtcaacatcagccagaatgattacattatctatgacaacaagtacaagaacttctctatttcgttctgggtacgcattcctaattacgataataagatcgtaaacgtgaacaatgaatatacgatcattaattgtatgcgtgataacaattccgggtggaaggtctcgctgaaccacaatgaaattatctggacgctgcaggacaacgctggtattaaccagaagctggccttcaattacggaaacgccaatggcattagcgattacattaacaaatggatttttgtgacaatcaccaatgatcgactaggcgattctaaattgtacattaatggcaatcttattgatcagaagtctatcttgaacctcggcaatatccacgtctccgacaacatacttttcaaaatagtgaactgctcctacaccagatacattggcatccgttactttaatatcttcgataaggagctggacgagactgagattcagaccctgtattccaacgagccaaacacaaacatactaaaagacttctggggcaattatttgctgtacgacaaggaatactatcttctgaacgtgttgaagcccaacaattttatagatcggaggaaggattcgactctgtcaattaacaatattagatcgacgatcctcctggcgaaccgcttgtattctggtatcaaagttaaaatccagcgtgttaacaattcctcgactaacgacaacttagtacggaagaacgaccaagtgtatattaatttcgtggcctccaagacccacctatttcccctgtacgctgatacggccacgactaacaaggagaagacgataaagatttcgtctagcggtaatcgctttaaccaggttgtagtgatgaatagcgtaggaaacaattgcactatgaactttaaaaacaataacggaaacaacatcggactgctgggcttcaaggccgatactgttgttgcctcgacgtggtactacacgcacatgcgagatcatacgaacagcaacggctgcttttggaacttcatttccgaagagcacggctggcaggagaagtaa

SEQ ID NO: 58 - DNA, 3759 bp, Artificial Sequence
  mat_peptide (1)...(3756): BoNT/E, D. melanogaster-modified 3
atgcctaagatcaatagcttcaactacaatgaccctgtcaacgacagaacaatcttgtacatcaagcccgggggctgccaggaattttacaaaagcttcaatattatgaaaaacatctggataatcccagagcggaacgttatcggcaccacgccacaggactttcatccgccaacttccctgaagaacggagattcgtcatattacgacccgaactatttgcagtccgacgaagagaaggatagatttctaaagatcgttactaagatatttaaccgaattaacaataacctaagcggcggaatcctgcttgaggaactgagtaaggctaacccgtacctaggcaacgataatacccccgacaaccagttccatatcggcgatgcgagcgccgttgaaataaagttttctaacggttctcaggacatacttttgccaaatgtgattataatgggcgcggagcccgacctctttgaaacgaattccagcaacattagcttgcgaaacaattatatgccttccaaccacggcttcggttccatcgcaattgtgaccttttcgccagagtattcgtttcgcttcaacgacaactcgatgaatgagttcatccaggaccccgctctgacgctgatgcacgaactgattcatagcctccacggtctttacggggccaaggggatcaccacaaaatacaccataacacagaagcagaacccgctgattacgaacattaggggtaccaacatagaagagttcctcaccttcggagggaccgatcttaatatcattactagcgcacagagcaatgatatttataccaacttgctggccgactacaaaaagatcgcctccaagctgagcaaagtgcaggtaagcaatccgctgctaaacccctataaagacgtcttcgaggccaaatacggactggataaggacgctagtggcatatactcggtcaatattaataaattcaacgatatcttcaagaaactgtattctttcacggagtttgatctggctacgaaatttcaggtgaagtgccgacagacatatatcggtcaatacaagtacttcaaactgagcaacctcttgaatgacagtatctataatatctccgagggatacaacattaataacctgaaggttaatttcaggggccaaaacgcaaatctgaacccacgcataatcacgcccattacaggacggggtctagtcaaaaagataatccgcttctgcaagaatatcgtgagtgtgaaaggcattaggaagtccatctgcattgagataaacaatggcgagttgtttttcgtggcttccgagaacagttataatgatgacaacatcaatacaccgaaagagatcgatgacaccgtgacctccaataacaattacgaaaatgacctggaccaggtcatcctgaatttcaactccgagagcgccccaggtctctccgatgaaaagctgaatctaacaatacagaacgatgcctacatccccaagtacgatagcaatggcacctccgatattgagcaacatgatgttaatgagctcaatgtattcttttatctcgatgcgcagaaagtgccggaaggcgagaataacgtcaacctaacctcgtctattgacaccgcactgttagaacaaccaaagatctatacttttttcagcagtgagttcattaataacgtgaacaagccggtccaagccgcattgttcgtgagctggatacagcaagtgctcgtggactttaccacggaggcaaaccagaagtccaccgtcgataagatcgctgatatatccattgtggtaccatacatcggcttggccctgaatattggaaatgaggcccagaagggaaactttaaggacgcgcttgagctgctcggcgccggcatcctgttggagttcgagccggaactgttgatccccaccatcctcgtgttcacgattaagtccttcctgggatcgtcagacaacaagaataaagtaatcaaggccattaataacgccttaaaggagcgcgatgagaaatggaaggaagtttactcgttcatagtttcgaactggatgacgaaaattaatacgcaatttaacaaacgaaaggaacaaatgtaccaagccctccagaatcaggtcaatgcgatcaagactataatcgaaagcaagtataactcgtacaccctggaggaaaaaaacgagttgacaaacaagtacgatattaaacagatcgagaatgagttgaaccagaaggtctcaattgcgatgaacaatatagacagattcttaaccgaaagctccatcagttaccttatgaagttgattaacgaagtgaagattaataagctgcgtgaatacgatgagaacgtgaaaacttacctgcttaattatatcatacaacacggaagtattctgggcgagagccagcaagagctgaatagtatggtgacggataccctgaacaattcaataccttttaagctttcttcctataccgatgacaagatcctcatcagttactttaacaagttctttaagaggattaagtcttcctcagtgctgaatatgcgctataagaacgataaatacgtggatacttccggatacgactcaaatatcaacatcaacggagacgtgtataaatatcccacgaataagaatcagttcggtatttacaatgataaactgtcggaggtaaacattagccaaaacgactatatcatatatgacaataaatataagaacttctcaatttcgttttgggtacgcataccgaactacgataataagatagttaatgtcaacaatgagtacacaatcattaactgcatgcgcgataataactccggatggaaagtatcgctgaatcacaacgagatcatatggacacttcaagataacgctggcatcaaccagaagttggcctttaactacggcaacgccaacggcattagtgattacattaacaagtggatcttcgtgacgatcactaacgatcgcctgggtgactcgaagctgtacattaatggcaacctaatcgaccagaagtcgattctgaatctgggaaacatccacgttagcgacaacatcctgttcaagattgtcaattgtagctacactcgctacataggtatccgttattttaatattttcgataaggagcttgacgagacagaaatccagaccctctacagtaacgaacccaacacgaatatactaaaggatttctggggcaattacttgctgtacgataaggagtactatctcctgaacgttttgaagcccaataacttcatcgatcggcgtaaggacagcactttaagtatcaacaatatccggagcaccatcctcttggcaaatcgtctgtactcgggtatcaaggttaaaattcagcgggtgaacaattcttcgaccaatgataatttagtgcgtaagaacgaccaggtgtatatcaacttcgtggcgtcgaagacgcacctctttcccctgtacgccgatacggctacaactaacaaggagaagaccattaaaatctcctcgtcaggtaatcgcttcaatcaggtcgtggtcatgaactcggtcggcaacaattgtaccatgaacttcaagaacaataacggcaacaatattggattgctgggctttaaggccgacacggtggtggcgtcaacatggtactatacacacatgcgagatcatactaactctaacggatgcttctggaacttcatttccgaggagcatggatggcaggagaagtaa

SEQ ID NO: 59 - DNA, 3759 bp, Artificial Sequence
  mat_peptide (1)...(3756): BoNT/E, S. frugiperda-modified 1
atgcctaaaatcaactcgttcaactacaacgaccccgtgaatgatcgaacgatcctgtacatcaagcccgggggctgccaggaattctacaaatcttttaacatcatgaagaacatctggatcattccagagcggaatgtcatcggtaccacaccgcaagacttccacccgcccacctctctaaagaacggtgacagttcgtactatgatccaaactacttgcagtccgacgaagagaaagaccgctttctcaagatcgtgacgaaaatttttaacaggatcaataacaatctgtcgggtggaatacttttggaggaactctccaaagccaacccctacctgggaaacgacaacacaccagacaaccagtttcatatcggcgacgctagcgccgtagagattaagttctccaacggatcgcaggacatcttgctgccaaacgtgatcattatgggtgcggagcccgacctgttcgaaacaaactcctctaacatctcattacgtaataactatatgccaagtaatcacggcttcggttctatcgctatcgtgactttcagtccagagtattcatttcgcttcaatgacaattccatgaacgagttcatccaggatcccgccttaactctgatgcacgagcttattcattctctgcatggcctgtacggtgccaagggtatcaccactaagtacaccattacacagaagcaaaaccccctaattaccaatatccggggaaccaacatagaagagtttctgacttttggaggtaccgacttgaatatcattacatcagcccagtctaacgatatctataccaatctgttggctgactacaagaaaatcgcatccaaactctcaaaggtgcaggtaagtaaccccctgctcaacccttacaaggatgtgttcgaggcaaagtacggcctcgataaggacgccagtggtatttattccgtcaacattaacaagttcaacgacatcttcaaaaagctttattcttttactgagtttgacttagctacaaagttccaagtgaagtgcaggcagacgtacattggtcagtacaagtacttcaagctgagtaacctgcttaatgactcaatttataacatctcggagggatacaacatcaacaatctcaaagtcaacttccgtggccagaatgcaaacttaaacccgcgtatcataactcctatcactggcagaggacttgtgaagaaaatcattaggttctgtaaaaacattgtaagcgttaaggggatccgtaagtcgatttgtattgaaatcaacaatggagaattattctttgtggcatccgagaattcatacaacgacgataacataaatacgcctaaggagattgacgatactgtcacttcgaataacaattatgagaacgacttggatcaggtgattctaaatttcaattctgaatcggctcctggcttgagcgacgaaaagctgaatctgacaatacagaatgatgcctacatcccgaaatacgattcaaacggcacttctgacatagaacaacacgacgtaaacgagctcaacgtcttcttttacttggatgcacaaaaagtccctgagggtgaaaacaatgttaaccttactagctcaatcgatacagctttgctggagcaaccaaagatctacaccttcttttcttcagagttcatcaataacgtcaacaagcctgttcaagcggccttgttcgtgagctggattcagcaagtcctcgtcgatttcaccacagaggctaatcaaaagtccaccgtggataaaatcgcggacatttccatcgttgtgccctatatcggactggctttgaacataggcaacgaagctcaaaaaggaaactttaaggacgccctagaacttctgggtgcaggaatcctcctggaattcgaaccagagctgttgatccccactattctggtgttcactatcaagagttttctgggctcttcggataacaaaaataaagttattaaagctatcaacaatgcgctcaaggagcgtgatgaaaagtggaaagaggtctattctttcattgtgtcaaattggatgactaagattaacacgcaatttaacaagagaaaggagcagatgtaccaggcattgcagaaccaggttaacgctattaagaccatcatagagagcaagtataactcatacacattggaagagaagaatgagttgacgaataaatatgacatcaaacaaatcgaaaacgagctaaaccagaaggtcagcatcgcgatgaacaatatcgaccgtttcctaacggagtccagcatctcttacttgatgaagctcatcaacgaggtaaagataaacaagttacgcgagtacgatgaaaacgtgaaaacgtacttgctcaactacatcatacagcatggttctattctgggtgagagccaacaggaattgaactccatggtcaccgacacccttaacaattccattccgttcaagcttagctcttatacggacgataaaatcctcattagctacttcaacaagttctttaagagaatcaagagctccagtgtgctaaacatgaggtacaagaacgataagtacgtcgacacctccggatatgattccaatatcaatatcaatggcgacgtttacaagtaccctaccaacaagaaccaattcggtatctacaacgacaagctttccgaggtaaatatcagtcaaaacgactacattatctacgacaacaaatacaagaacttctcgatctccttctgggtgcgcatccctaactacgacaacaagatcgtaaacgttaataacgagtacaccataatcaactgcatgagagacaataactccggctggaaggtctcgttgaatcacaatgaaatcatttggactttgcaggataatgctggcatcaaccagaaactcgccttcaactacggtaacgctaacggcattagcgactacatcaataagtggatcttcgttaccattaccaacgatcgcctcggagattcaaagctctatatcaacggtaacctcatcgaccaaaagagcattctaaacctcggaaacatccacgtatccgacaacatcctgttcaagatagttaactgctcatacactaggtacattggcatcaggtacttcaacatcttcgacaaggaactcgacgaaacggaaatacaaactctgtatagtaacgaacccaacaccaacatcctcaaggacttctggggcaactaccttctctacgacaaagagtactatctcctgaatgtccttaaaccgaacaatttcatcgaccgccgtaaggattccacactgtccatcaacaatattagatcaacaatcctgttagcgaaccgtctgtacagcggtattaaggttaagatacagcgcgtgaataactcatctacgaacgataaccttgtccgaaagaacgaccaggtgtacatcaatttcgtagcctccaagacccacctgttccctctctacgccgacactgctacaaccaacaaggagaaaaccatcaaaatatcgagtagcggtaacaggttcaatcaggtggtcgttatgaactctgtcggaaacaactgtactatgaacttcaagaacaacaacgggaacaacattggtctgctgggattcaaggcggatacagtcgtggctagcacatggtactacacccacatgcgcgaccataccaactccaacggttgcttctggaacttcatttctgaagagcacggctggcaggagaaataa

SEQ ID NO: 60 - DNA, 3759 bp, Artificial Sequence
  mat_peptide (1)...(3756): BoNT/E, S. frugiperda-modified 2
atgcccaagatcaactccttcaactacaacgaccccgtgaacgaccgtaccatcctgtacatcaagcccggtggttgccaggagttctacaagtccttcaacatcatgaagaacatctggatcatccccgagcgtaacgtgatcggtaccaccccccaggacttccacccccccacctccctgaagaacggtgactcctcctactacgaccccaactacctgcagtccgacgaggagaaggaccgtttcctgaagatcgtgaccaagatcttcaaccgtatcaacaacaacctgtccggtggtatcctgctggaggagctgtccaaggctaacccctacctgggtaacgacaacacccccgacaaccagttccacatcggtgacgcttccgctgtggagatcaagttctccaacggttcccaggacatcctgctgcccaacgtgatcatcatgggtgctgagcccgacctgttcgagaccaactcctccaacatctccctgcgtaacaactacatgccctccaaccacggtttcggttccatcgctatcgtgaccttctcccccgagtactccttccgtttcaacgacaactccatgaacgagttcatccaggaccccgctctgaccctgatgcacgagctgatccactccctgcacggtctgtacggtgctaagggtatcaccaccaagtacaccatcacccagaagcagaaccccctgatcaccaacatccgtggtaccaacatcgaggagttcctgaccttcggtggtaccgacctgaacatcatcacctccgctcagtccaacgacatctacaccaacctgctggctgactacaagaagatcgcttccaagctgtccaaggtgcaggtgtccaaccccctgctgaacccctacaaggacgtgttcgaggctaagtacggtctggacaaggacgcttccggtatctactccgtgaacatcaacaagttcaacgacatcttcaagaagctgtactccttcaccgagttcgacctggctaccaagttccaggtgaagtgccgtcagacctacatcggtcagtacaagtacttcaagctgtccaacctgctgaacgactccatctacaacatctccgagggttacaacatcaacaacctgaaggtgaacttccgtggtcagaacgctaacctgaacccccgtatcatcacccccatcaccggtcgtggtctggtgaagaagatcatccgtttctgcaagaacatcgtgtccgtgaagggtatccgtaagtccatctgcatcgagatcaacaacggtgagctgttcttcgtggcttccgagaactcctacaacgacgacaacatcaacacccccaaggagatcgacgacaccgtgacctccaacaacaactacgagaacgacctggaccaggtgatcctgaacttcaactccgagtccgctcccggtctgtccgacgagaagctgaacctgaccatccagaacgacgcttacatccccaagtacgactccaacggtacctccgacatcgagcagcacgacgtgaacgagctgaacgtgttcttctacctggacgctcagaaggtgcccgagggtgagaacaacgtgaacctgacctcctccatcgacaccgctctgctggagcagcccaagatctacaccttcttctcctccgagttcatcaacaacgtgaacaagcccgtgcaggctgctctgttcgtgtcctggatccagcaggtgctggtggacttcaccaccgaggctaaccagaagtccaccgtggacaagatcgctgacatctccatcgtggtgccctacatcggtctggctctgaacatcggtaacgaggctcagaagggtaacttcaaggacgctctggagctgctgggtgctggtatcctgctggagttcgagcccgagctgctgatccccaccatcctggtgttcaccatcaagtccttcctgggttcctccgacaacaagaacaaggtgatcaaggctatcaacaacgctctgaaggagcgtgacgagaagtggaaggaggtgtactccttcatcgtgtccaactggatgaccaagatcaacacccagttcaacaagcgtaaggagcagatgtaccaggctctgcagaaccaggtgaacgctatcaagaccatcatcgagtccaagtacaactcctacaccctggaggagaagaacgagctgaccaacaagtacgacatcaagcagatcgagaacgagctgaaccagaaggtgtccatcgctatgaacaacatcgaccgtttcctgaccgagtcctccatctcctacctgatgaagctgatcaacgaggtgaagatcaacaagctgcgtgagtacgacgagaacgtgaagacctacctgctgaactacatcatccagcacggttccatcctgggtgagtcccagcaggagctgaactccatggtgaccgacaccctgaacaactccatccccttcaagctgtcctcctacaccgacgacaagatcctgatctcctacttcaacaagttcttcaagcgtatcaagtcctcctccgtgctgaacatgcgttacaagaacgacaagtacgtggacacctccggttacgactccaacatcaacatcaacggtgacgtgtacaagtaccccaccaacaagaaccagttcggtatctacaacgacaagctgtccgaggtgaacatctcccagaacgactacatcatctacgacaacaagtacaagaacttctccatctccttctgggtgcgtatccccaactacgacaacaagatcgtgaacgtgaacaacgagtacaccatcatcaactgcatgcgtgacaacaactccggttggaaggtgtccctgaaccacaacgagatcatctggaccctgcaggacaacgctggtatcaaccagaagctggctttcaactacggtaacgctaacggtatctccgactacatcaacaagtggatcttcgtgaccatcaccaacgaccgtctgggtgactccaagctgtacatcaacggtaacctgatcgaccagaagtccatcctgaacctgggtaacatccacgtgtccgacaacatcctgttcaagatcgtgaactgctcctacacccgttacatcggtatccgttacttcaacatcttcgacaaggagctggacgagaccgagatccagaccctgtactccaacgagcccaacaccaacatcctgaaggacttctggggtaactacctgctgtacgacaaggagtactacctgctgaacgtgctgaagcccaacaacttcatcgaccgtcgtaaggactccaccctgtccatcaacaacatccgttccaccatcctgctggctaaccgtctgtactccggtatcaaggtgaagatccagcgtgtgaacaactcctccaccaacgacaacctggtgcgtaagaacgaccaggtgtacatcaacttcgtggcttccaagacccacctgttccccctgtacgctgacaccgctaccaccaacaaggagaagaccatcaagatctcctcctccggtaaccgtttcaaccaggtggtggtgatgaactccgtgggtaacaactgcaccatgaacttcaagaacaacaacggtaacaacatcggtctgctgggtttcaaggctgacaccgtggtggcttccacctggtactacacccacatgcgtgaccacaccaactccaacggttgcttctggaacttcatctccgaggagcacggttggcaggagaagtaa

SEQ ID NO: 61 - DNA, 3759 bp, Artificial Sequence
  mat_peptide (1)...(3756): BoNT/E, S. frugiperda-modified 3
atgccgaagatcaattccttcaactacaacgaccccgtcaatgatcgcaccatcctatacatcaagcctggcggatgtcaggagttttacaaatcattcaacatcatgaagaacatttggattatcccagagcgtaacgtaattggtactacccctcaggatttccaccccccaacctcattgaaaaacggcgactcttcgtattacgaccccaactacctacaatccgacgaagagaaggaccgtttcttgaagatcgtcaccaagattttcaacagaattaacaataacttgtctggaggtatactacttgaggaattatcgaaggccaatccgtatttgggtaacgacaacactcccgacaaccaattccacatcggagacgcgtcagcagtggaaatcaagttctctaacggctcccaagacatccttctgccgaacgtgataatcatgggagccgaacctgacctgttcgagaccaactcgtctaatatcagtctccgtaataactacatgccttcaaaccacggctttggaagcatcgccattgtcaccttctcacctgaatattcattccgctttaacgacaacagcatgaatgagttcattcaggaccccgctttgaccttgatgcacgagctcatccatagtttgcatggtctgtacggtgcaaagggaataacgacaaaatatacaatcacccagaagcagaaccctctgatcactaacatcaggggaactaatattgaagagttcctaaccttcggcggtaccgacctgaacattatcaccagcgctcaaagcaacgatatttatacgaatctgctcgctgattacaagaaaatcgcttcgaagttgagtaaggtccaagtttcaaatccgctgcttaacccttacaaagatgttttcgaggccaagtacgggttggacaaggacgctagtggcatctactccgtgaacattaacaagttcaatgatatcttcaagaaactttactcatttaccgagttcgatctggcgacaaaattccaggtcaaatgtagacagacttatattggccaatacaaatactttaaactttcgaatctactgaatgatagtatctacaacatttctgagggttacaatataaataacctgaaagttaactttcgcggtcagaacgcaaacctgaacccacgtatcattactcctataactggcagaggtttggtgaaaaagattatccgattctgcaaaaatatcgtctcggtcaaaggcatccgcaagtctatctgcatcgagatcaacaatggagaactgttctttgtcgcgtctgaaaacagctacaacgatgacaacataaacacaccaaaggaaattgacgatacagtaacgtctaacaataactacgagaatgaccttgatcaggtgatcctgaacttcaattctgagtccgcgccaggcctctcggacgagaagttgaatctgacgattcagaacgacgcctacatcccgaagtatgactcgaacggcacatccgacatcgagcaacacgatgtgaacgagctgaacgttttcttttacctggacgcacagaaggtgcccgagggtgaaaataacgtgaacttaacatccagcattgataccgccctgttggagcaacctaagatctacacttttttctctagcgaatttattaacaatgtaaataaacccgtccaagctgcactattcgtctcatggatccaacaggtgttagtcgatttcacaaccgaggccaatcagaagtccactgttgataaaatcgctgacatctccatcgtcgttccctacatcggtctcgctctgaacattggcaacgaagcccagaagggaaatttcaaggatgccctggaactgttaggggccggcatcttactcgagtttgaacccgaactgttaattccaacaattctcgtctttaccattaaatccttcctcggtagttcagacaacaaaaacaaagtaataaaggctattaacaatgcattgaaggaacgtgatgagaaatggaaggaggtatattccttcattgtgtctaactggatgaccaagatcaatactcagttcaacaaaaggaaagaacagatgtatcaggcgttgcagaaccaggtgaacgccatcaagacaattatcgaaagcaaatataactcctacacactggaggaaaaaaacgaacttactaacaagtacgacattaagcaaatcgagaacgagctcaaccaaaaggtctccatcgctatgaacaatatcgatcgtttcctgactgagtcctctatttcctaccttatgaagctcatcaacgaggtaaagatcaataagctgcgcgaatacgatgagaacgtcaagacgtatttgcttaactatatcatacagcacggtagcatcctcggtgagagtcaacaggaactcaacagcatggtaaccgacactttgaacaattccatcccattcaaactctccagctacactgatgacaagatcctcatcagctacttcaacaagttctttaaaagaattaagtcctcttcggtgctcaatatgcggtacaagaacgacaagtacgtagacacatcaggatacgacagtaacataaacattaatggcgacgtttacaagtaccccacaaacaaaaaccagttcggtatctataacgacaagctctccgaagtgaatatctctcagaatgattacataatctacgacaacaagtacaagaacttcagcatttctttctgggtcaggattcctaactatgacaacaagatcgtgaacgttaacaatgaatacactataatcaactgcatgcgtgacaataactcaggctggaaggtcagcctaaaccataacgaaatcatttggaccttgcaggacaacgctggcatcaaccaaaagcttgcgttcaactacggaaatgctaatgggataagcgactacattaacaagtggatcttcgtgactataaccaacgatcgcttgggagatagcaagctgtacatcaacggaaacctaatagatcaaaagtccatccttaacctgggtaacatccacgtgtcagacaacattcttttcaagattgttaactgcagttataccaggtacatcggaatccgctacttcaacattttcgacaaggagctggatgagaccgagatccaaacattgtactctaacgagccaaacactaacatcctgaaggatttctggggtaactatctgctctacgataaagagtattacctcctgaacgtactgaagccgaacaatttcatcgaccgaaggaaggactcgacactctctatcaacaatatacgcagcacgatcctcttggctaacaggctgtactccggtatcaaggtgaagatccagagagttaacaatagttccactaacgacaatctcgtgcgtaagaatgaccaggtttacatcaatttcgtcgcttcaaaaacccatttattccccctttacgcagacacggcgactacgaataaggagaagacgattaagatctcgtcttccggaaacaggtttaatcaggttgtggtcatgaactcggtgggcaacaattgtacgatgaactttaagaacaataacggtaacaacatcggtctcctcggcttcaaggccgacacggtggtggcttccacctggtactacacccacatgcgggaccataccaacagtaacggttgcttttggaacttcatctcagaagagcacggatggcaagagaaataa

SEQ ID NO: 62 - DNA, 3759 bp, Artificial Sequence
  mat_peptide (1)...(3756): BoNT/E, D. rerio-modified 1
atgccaaagatcaactcgtttaactacaatgacccagttaacgatcgcactatcctttatatcaagcccggtggatgccaggagttttacaaatccttcaatatcatgaagaatatttggatcattcctgagcgtaacgtcattggaactacaccgcaggatttccaccctcccacgtctctgaaaaatggcgattcaagttactatgatccaaactatctgcagtctgatgaggaaaaagataggtttcttaaaatcgtgaccaagatcttcaacagaatcaacaataacctcagtggtggcattctgctagaagagcttagcaaagctaatccctacttggggaacgacaatacgccggataaccaatttcatataggcgacgcctctgcagtggagattaaattctcaaatggtagccaggatatattgctgcctaacgttattataatgggcgcggagccggacctatttgagacgaactcgtccaatatttccctccggaacaattatatgccgagtaaccacgggtttggcagtatagctattgtgacatttagtccagaatattcatttaggtttaacgacaactctatgaacgaattcatccaagatccagcattgactttgatgcatgagctgattcattcattgcacggcctgtatggtgccaaaggcattaccactaaatacactatcacccagaagcagaacccgctgattacaaacatcaggggcaccaacatagaagagttcctgacattcggaggtaccgaccttaacattatcacttcagcacagagtaacgacatctacaccaacctgcttgccgactataaaaagatcgcctcaaaactttccaaagttcaggtcagtaacccactgctaaatccatataaggatgtgttcgaagccaagtatggattggacaaggacgccagtggaatctactctgtaaatattaataaattcaacgacatatttaagaaactgtattctttcaccgagttcgacctggctactaagtttcaggtcaaatgtagacagacatacatcggccaatacaagtacttcaagctgtcaaacctgcttaacgatagtatttacaacattagcgaggggtataatatcaacaatctaaaggtgaacttccgtgggcagaacgccaacctcaacccacgcataatcacgcccatcactggtagagggctcgtcaaaaagatcattagattttgtaaaaacatcgtctctgttaagggtatccgcaaaagcatatgcatcgagatcaataacggcgagctcttctttgtggccagcgagaactcctataacgacgataatataaacacacctaaggaaattgatgacacagtcacctccaacaataactatgaaaacgacctggatcaggtcattctcaacttcaattcggaaagtgcgcctggactcagcgacgagaagctaaacctgactatccagaacgatgcatacattcccaagtatgattcaaacggtacatccgatatcgagcagcatgatgtgaatgaactgaatgtgttcttttatctggatgcgcaaaaggtgccagagggtgagaataacgtgaacctgacttctagcatcgatacggctctcctggagcagccaaaaatctacacatttttctcatcggagtttattaacaatgtcaataagcctgtgcaggcagcgctgttcgtctcatggatccaacaggtactcgtggactttaccacggaagctaatcagaaatctactgttgacaagattgcagacatctcaatcgtggttccttatatcggattggcactgaacattggtaacgaggcacaaaaaggcaacttcaaggacgctttggagttactcggagctggcatcctgttagagtttgaacccgagctcttgatccccacaatactggtgttcaccataaagtcattccttggcagctcagacaacaagaacaaggtaataaaagccattaacaatgcgctgaaggagcgagacgagaagtggaaagaagtctattcctttatagtcagcaattggatgaccaaaatcaacacccaattcaacaaacggaaagaacagatgtaccaggccttacagaatcaagttaacgctattaaaaccatcatagagagcaaatataactcgtataccctcgaggaaaaaaatgaactgactaataaatacgatattaaacagatcgaaaatgagctcaaccagaaagtgtctatcgctatgaataacatcgaccgctttctcactgagtcttccatctcatatctgatgaagctcattaatgaagttaaaatcaataagcttagagagtacgacgagaacgtgaagacctacttgctgaactacatcattcaacacggcagcatcctgggagagtcccagcaagaactaaactctatggtaaccgacacgctgaataacagcatacccttcaaactatcctcttacacagatgacaaaatcctgatcagctacttcaataagttctttaagcgaattaaatcctcaagtgtgctgaacatgcggtacaaaaacgacaagtacgtggacacatctgggtacgacagcaatatcaatattaatggagatgtttataagtaccccacaaacaagaaccaattcggaatctacaatgataagctttctgaggtgaatatctctcagaacgactatatcatatatgataacaaatacaaaaacttttctatcagcttctgggtgcgtatccctaactacgacaataagatcgttaacgtgaacaatgagtacacgatcattaactgcatgagagacaataacagtggttggaaggtttccttgaatcataatgaaatcatttggacgctgcaggataatgcagggataaatcagaagcttgcttttaattacggaaacgctaatggaatttccgactatataaacaagtggatcttcgtaactatcacaaacgaccgattaggagatagtaaactgtacatcaacgggaatctcattgatcagaaaagcatcctcaacctgggaaatattcacgtctcggacaatatcctttttaagatcgtgaattgtagctacacaagatacattggaattagatattttaacatctttgataaggaattggacgagaccgaaatccagactctttacagcaatgagcctaacaccaatattttgaaggatttttggggaaattacctgttatacgacaaggagtactatttactgaacgtgctgaaacctaacaattttattgatcgacgtaaggattccactctcagcattaacaatatccgcagcaccattctgcttgctaacaggctctattcgggaataaaagtcaagatccagagggtgaacaattctagtactaatgacaacctggtgcgcaagaatgatcaggtgtacattaacttcgtggcttctaagacccacttattccctctgtacgccgacactgcaacaaccaacaaagagaagacaattaaaatcagctccagtggcaacaggtttaatcaggtagtcgttatgaattccgtcgggaacaattgtacgatgaacttcaaaaacaacaacggtaacaacattggactgctgggattcaaagccgacacagttgtggcctctacatggtattacacacatatgcgggatcacacaaacagcaacggttgcttctggaacttcatctccgaggaacacggctggcaggagaagtaa

SEQ ID NO: 63 - DNA, 3759 bp, Artificial Sequence
  mat_peptide (1)...(3756): BoNT/E, D. rerio-modified 2
atgcctaagatcaacagcttcaactacaacgaccctgtgaacgacagaacaatcctgtacatcaagcctggaggatgtcaggagttctacaagagcttcaacatcatgaagaacatctggatcatccctgagagaaacgtgatcggaacaacacctcaggacttccaccctcctacaagcctgaagaacggagacagcagctactacgaccctaactacctgcagagcgacgaggagaaggacagattcctgaagatcgtgacaaagatcttcaacagaatcaacaacaacctgagcggaggaatcctgctggaggagctgagcaaggctaacccttacctgggaaacgacaacacacctgacaaccagttccacatcggagacgctagcgctgtggagatcaagttcagcaacggaagccaggacatcctgctgcctaacgtgatcatcatgggagctgagcctgacctgttcgagacaaacagcagcaacatcagcctgagaaacaactacatgcctagcaaccacggattcggaagcatcgctatcgtgacattcagccctgagtacagcttcagattcaacgacaacagcatgaacgagttcatccaggaccctgctctgacactgatgcacgagctgatccacagcctgcacggactgtacggagctaagggaatcacaacaaagtacacaatcacacagaagcagaaccctctgatcacaaacatcagaggaacaaacatcgaggagttcctgacattcggaggaacagacctgaacatcatcacaagcgctcagagcaacgacatctacacaaacctgctggctgactacaagaagatcgctagcaagctgagcaaggtgcaggtgagcaaccctctgctgaacccttacaaggacgtgttcgaggctaagtacggactggacaaggacgctagcggaatctacagcgtgaacatcaacaagttcaacgacatcttcaagaagctgtacagcttcacagagttcgacctggctacaaagttccaggtgaagtgtagacagacatacatcggacagtacaagtacttcaagctgagcaacctgctgaacgacagcatctacaacatcagcgagggatacaacatcaacaacctgaaggtgaacttcagaggacagaacgctaacctgaaccctagaatcatcacacctatcacaggaagaggactggtgaagaagatcatcagattctgtaagaacatcgtgagcgtgaagggaatcagaaagagcatctgtatcgagatcaacaacggagagctgttcttcgtggctagcgagaacagctacaacgacgacaacatcaacacacctaaggagatcgacgacacagtgacaagcaacaacaactacgagaacgacctggaccaggtgatcctgaacttcaacagcgagagcgctcctggactgagcgacgagaagctgaacctgacaatccagaacgacgcttacatccctaagtacgacagcaacggaacaagcgacatcgagcagcacgacgtgaacgagctgaacgtgttcttctacctggacgctcagaaggtgcctgagggagagaacaacgtgaacctgacaagcagcatcgacacagctctgctggagcagcctaagatctacacattcttcagcagcgagttcatcaacaacgtgaacaagcctgtgcaggctgctctgttcgtgagctggatccagcaggtgctggtggacttcacaacagaggctaaccagaagagcacagtggacaagatcgctgacatcagcatcgtggtgccttacatcggactggctctgaacatcggaaacgaggctcagaagggaaacttcaaggacgctctggagctgctgggagctggaatcctgctggagttcgagcctgagctgctgatccctacaatcctggtgttcacaatcaagagcttcctgggaagcagcgacaacaagaacaaggtgatcaaggctatcaacaacgctctgaaggagagagacgagaagtggaaggaggtgtacagcttcatcgtgagcaactggatgacaaagatcaacacacagttcaacaagagaaaggagcagatgtaccaggctctgcagaaccaggtgaacgctatcaagacaatcatcgagagcaagtacaacagctacacactggaggagaagaacgagctgacaaacaagtacgacatcaagcagatcgagaacgagctgaaccagaaggtgagcatcgctatgaacaacatcgacagattcctgacagagagcagcatcagctacctgatgaagctgatcaacgaggtgaagatcaacaagctgagagagtacgacgagaacgtgaagacatacctgctgaactacatcatccagcacggaagcatcctgggagagagccagcaggagctgaacagcatggtgacagacacactgaacaacagcatccctttcaagctgagcagctacacagacgacaagatcctgatcagctacttcaacaagttcttcaagagaatcaagagcagcagcgtgctgaacatgagatacaagaacgacaagtacgtggacacaagcggatacgacagcaacatcaacatcaacggagacgtgtacaagtaccctacaaacaagaaccagttcggaatctacaacgacaagctgagcgaggtgaacatcagccagaacgactacatcatctacgacaacaagtacaagaacttcagcatcagcttctgggtgagaatccctaactacgacaacaagatcgtgaacgtgaacaacgagtacacaatcatcaactgtatgagagacaacaacagcggatggaaggtgagcctgaaccacaacgagatcatctggacactgcaggacaacgctggaatcaaccagaagctggctttcaactacggaaacgctaacggaatcagcgactacatcaacaagtggatcttcgtgacaatcacaaacgacagactgggagacagcaagctgtacatcaacggaaacctgatcgaccagaagagcatcctgaacctgggaaacatccacgtgagcgacaacatcctgttcaagatcgtgaactgtagctacacaagatacatcggaatcagatacttcaacatcttcgacaaggagctggacgagacagagatccagacactgtacagcaacgagcctaacacaaacatcctgaaggacttctggggaaactacctgctgtacgacaaggagtactacctgctgaacgtgctgaagcctaacaacttcatcgacagaagaaaggacagcacactgagcatcaacaacatcagaagcacaatcctgctggctaacagactgtacagcggaatcaaggtgaagatccagagagtgaacaacagcagcacaaacgacaacctggtgagaaagaacgaccaggtgtacatcaacttcgtggctagcaagacacacctgttccctctgtacgctgacacagctacaacaaacaaggagaagacaatcaagatcagcagcagcggaaacagattcaaccaggtggtggtgatgaacagcgtgggaaacaactgtacaatgaacttcaagaacaacaacggaaacaacatcggactgctgggattcaaggctgacacagtggtggctagcacatggtactacacacacatgagagaccacacaaacagcaacggatgtttctggaacttcatcagcgaggagcacggatggcaggagaagtaa

SEQ ID NO: 64 - DNA, 3759 bp, Artificial Sequence
  mat_peptide (1)...(3756): BoNT/E, D. rerio-modified 3
atgcccaagatcaattcctttaactacaacgaccctgtcaatgaccggacaattttgtacatcaagcctggagggtgccaggagttctataagagcttcaacattatgaaaaatatttggatcattcctgaaagaaacgtcatcggcaccacaccgcaggatttccaccctcccactagcctgaagaacggcgattctagctattacgaccctaattaccttcagtcagacgaggaaaaggatcgttttcttaagatcgtgacaaagatctttaaccgaatcaacaataacttgtcaggcggtatccttctggaggaactgtctaaggcgaacccatatctgggcaacgataacactccagataaccagtttcatatcggcgatgctagcgctgtggaaattaaattcagtaacggctcccaggacattctcctgcctaatgtgatcattatgggtgctgagcctgacctcttcgagaccaatagctcaaacatttctctgagaaacaattacatgccatcaaatcacgggttcggaagtattgctatcgtcacgttcagcccggagtattcatttcgatttaacgacaatagtatgaacgagttcatccaggaccctgctttgacactcatgcatgagcttatccactctcttcacggattgtacggggcaaaaggtatcactacaaagtacaccatcacgcagaagcagaatccactgatcaccaacatcagaggcacgaatattgaagagttccttacctttggagggacagacctgaacatcattacttctgcccagagcaacgacatctatactaacctgttggcagattacaagaaaatcgcaagtaagctgagtaaagtgcaggtctcaaaccccctgctcaacccatataaggacgtcttcgaggccaaatatggactggataaggacgcatcaggaatctacagcgtgaatatcaacaaatttaacgatattttcaagaaactgtattcctttacagagtttgaccttgctaccaagttccaggtgaagtgcaggcagacgtacatcggtcagtacaagtacttcaaattgagcaatctgttgaatgactcaatctataatattagcgagggctataacatcaataacctgaaagtcaattttcgtggtcagaacgccaatctcaaccccaggattatcacaccaatcactggccggggactcgtgaagaaaatcattcgcttctgtaagaacatcgtgtctgtgaagggaatccgtaaatccatttgcatcgagatcaataacggcgaactgttctttgtggctagtgagaattcctacaacgacgataacatcaacacgccgaaagaaatcgacgatactgttacatccaacaataactatgagaatgatctggaccaggttattcttaacttcaactctgagtcagcaccaggactgagcgatgagaaactcaaccttacaatccagaacgatgcatacattcccaaatacgactctaacggaacctccgacattgaacagcacgacgtgaatgaactgaatgtgttcttttacctggatgctcagaaggtgccagaaggagagaacaatgtgaacctgacgagctccatcgacacagccctcctggagcagcccaagatctacacattctttagcagtgagttcatcaataacgttaacaagccggtccaggccgctctctttgtgtcttggatccagcaggtcctggttgactttactacagaagcaaatcagaagtccactgttgataaaatcgcggacatcagcatcgttgtcccttacatcggactggccctgaatatcggaaacgaggctcagaaggggaactttaaagacgcgctggagctgttgggcgctggtatcctgctcgagtttgagcccgagctgttgatccctactattcttgtgtttacgatcaaatcctttctgggtagctcagataataagaataaagttatcaaggctattaacaatgccctcaaagaaagagacgagaagtggaaggaggtttattctttcatcgtgagtaattggatgaccaagattaacacacagtttaataaacgaaaggagcagatgtaccaggccctccagaaccaggtcaacgccatcaagaccattatcgagagcaagtacaactcttatacactggaagagaaaaacgagctgacaaacaagtatgatattaaacagatcgagaatgagctgaaccagaaggtttctatcgcgatgaacaatatcgacagatttctgacggaaagctctatctcctacctgatgaaactgatcaatgaagttaaaatcaataagctgagggagtatgacgaaaacgtcaaaacctacctcctgaattatattatccagcatggaagcattttgggagaatcacagcaggaactgaactccatggtgaccgacactttgaacaatagcatcccattcaagctcagcagttacacagatgacaagattctgattagttatttcaacaagtttttcaagagaattaagtcctctagcgtgctgaacatgagatacaagaatgataaatacgttgacacatctggctacgatagtaacatcaacattaacggggacgtctacaaataccccaccaataaaaaccagttcggcatctacaacgataaactgtcagaggtgaacatcagtcagaacgattatatcatttacgataataaatataaaaatttctccatcagtttctgggtcagaatccctaattacgataataagattgtgaatgtgaataacgaatataccattatcaactgcatgagggacaataactctggctggaaagtgagcctcaaccataacgagatcatttggactctgcaggataacgccggaatcaatcagaagctcgccttcaattacgggaatgcaaatgggattagcgattacatcaacaaatggatttttgttacaatcaccaacgaccggcttggtgactctaagctgtatatcaatggtaacctgattgatcagaaatccatcctgaaccttgggaatatccacgtgtcagataacattctgtttaagatcgtgaattgttcttacaccaggtacatcggtatcaggtatttcaatattttcgataaagagctggatgagaccgaaattcagaccctgtattcaaacgagccaaacactaacattctgaaagacttttggggcaactacttgctgtacgacaaggaatactatctgttgaacgtcctcaagccgaataacttcatcgatcgccgtaaagatagcaccctgtccattaacaatatccgctccacaatcctgcttgcgaatcgactgtactctggtattaaagtgaaaatccagcgggtgaacaatagctccaccaacgacaacctggtccgcaagaacgatcaggtgtatatcaactttgtggcatctaaaacccaccttttccccctgtatgccgacacagctacgactaacaaagaaaaaacgatcaagatcagctcaagtggtaatcgcttcaatcaggtggttgtgatgaacagtgtgggaaacaattgtactatgaacttcaaaaacaataacggcaacaacatcggactcctcggtttcaaagcagacactgtggtggccagcacttggtattatactcatatgcgcgaccataccaacagcaacggatgtttttggaactttatcagcgaggaacacggatggcaggagaaataa

SEQ ID NO: 65 - DNA, 3759 bp, Artificial Sequence
  mat_peptide (1)...(3756): BoNT/E, X. laevis-modified 1
atgccaaaaattaactctttcaattataacgacccagtgaacgatcggactatcttgtacatcaaacctgggggatgtcaggagttttataaatccttcaacattatgaagaatatatggattatccctgaaaggaatgtaataggtactacaccccaggactttcacccaccgacctccctaaagaatggagattctagctattacgatcctaactacctacagagtgacgaagagaaagataggtttctgaagattgtcacaaagatcttcaacaggataaataacaatctaagcgggggcattcttttggaggaactgagcaaggccaatccctatctgggaaacgacaatactcccgataaccagtttcatataggagacgctagcgctgtggagatcaagttctcaaatggatcccaagatattctcttaccaaacgttattatcatgggtgccgagccggatctcttcgaaactaacagctcaaacatatctctgaggaacaattatatgccatctaaccacgggtttggaagtattgctatcgtgaccttttcacctgaatatagttttagatttaatgacaattctatgaatgagttcatccaagaccccgcactaactctcatgcacgagttaattcactcattacatggcctgtatggagcaaaggggataaccacgaagtacaccatcacccagaaacagaacccactcatcactaacatccgaggaaccaatattgaagagttcctgaccttcggagggaccgatctgaacatcattaccagtgctcaatctaacgatatttatacaaatctgctagctgattacaaaaagatagccagtaagttaagcaaggtgcaggttagtaacccactacttaatccgtacaaagacgtgttcgaggctaaatacggtttagataaagacgcatccggtatttactcggttaacatcaataagttcaatgatattttcaagaaactttatagcttcacagagtttgacttggctaccaaatttcaggtgaaatgccgacagacgtatatcggacagtataagtacttcaaattgtccaacttgctcaatgactccatttacaacattagtgaaggctataatatcaacaatttgaaagttaactttaggggccaaaatgccaacctgaaccctagaataatcacacctattacaggccgcggccttgttaagaaaatcattagattttgtaaaaatattgtatctgttaagggaatccgcaaatccatttgcattgaaattaataacggagaactcttctttgtcgcttctgagaactcctataacgatgacaacatcaacacgccaaaagagatagacgatacagtcacgtctaataacaattacgagaatgacttggaccaagtaatcttgaattttaatagtgaatcggcacctggtttgtctgatgagaaacttaatctgacaatacagaacgatgcatacataccaaaatacgactccaatgggactagtgacattgagcagcacgatgtgaacgaacttaacgtttttttctatctagacgcgcagaaggtccccgaaggtgaaaataacgtgaatctcacatcatctatagatacagcacttttagaacagccaaaaatctacacatttttctcatcggaatttatcaataacgtgaataagcctgtgcaagccgcactctttgtctcttggatacaacaggtgttagttgatttcactacagaagctaatcaaaaaagtacagtggacaagatagccgacatttcgattgtagttccctatatcggactggcactcaatatcggtaatgaggctcagaaaggaaatttcaaggacgccctggagcttttgggggccggcattttgctcgaatttgaaccagagctgctaattcctactattctggtcttcacaattaagtcctttctaggcagttcagataacaagaataaagtgattaaggcaattaataacgcccttaaagaaagagatgagaaatggaaggaggtgtacagcttcatcgtctcaaactggatgactaagatcaacactcaatttaataagcgaaaagagcaaatgtatcaggctcttcaaaatcaagtgaacgccatcaagactatcattgaatctaaatataattcatacacactggaggaaaagaacgaattaaccaataaatacgacatcaaacaaatcgagaatgaactcaaccagaaggtaagcatcgcaatgaacaatattgatcgttttcttactgagtcttccatttcatatttaatgaaactgatcaacgaagtgaagatcaataagctccgggaatacgatgaaaacgtcaaaacttatctgttgaactacataattcagcacggctcaatccttggagaatctcagcaagagttaaatagcatggttactgacacacttaataacagcatacccttcaaattgagttcctacacagacgataagattcttatttcctattttaataaatttttcaagcgtataaagtcatcctcagtactgaacatgcgctacaaaaatgataagtacgtagacacatctggttatgattctaatataaatatcaatggagatgtctacaagtacccaactaacaaaaatcagtttggcatttataatgacaaactttcggaggtaaacatttcccaaaacgactacataatttacgataataaatataagaacttttccattagcttttgggtcagaatacccaactacgataataaaatcgttaatgtcaacaatgaatataccattataaattgtatgcgtgataataactccggttggaaggtttcactcaatcacaatgaaattatctggacattgcaggataacgctggaattaaccagaaactggcctttaactacggaaacgcaaacggtattagcgattatatcaataaatggatattcgtgactataaccaatgatcggcttggcgacagcaagctgtatataaatgggaaccttatcgatcagaagtctatccttaatctggggaatatccatgtgagcgacaacatcctgttcaagatagtgaactgcagctacacacggtatataggcatcaggtattttaacattttcgataaagaattagatgaaaccgagattcagacactgtactcaaatgaaccaaacaccaatatattgaaagatttttgggggaactatctgctatacgacaaagagtattacctgctcaatgtgctgaaacctaataactttatcgacagaaggaaggatagtactcttagcattaacaatattagaagtaccatcctgctcgcaaacagattgtatagtggtattaaagtcaaaatacagcgcgtaaataactcttcaactaacgataatctggtgcgaaaaaatgatcaagtatatatcaattttgtagcgagcaagacacatctgttccctttgtacgctgataccgcgacaaccaataaagagaagacgataaagatttctagttctggcaaccgttttaaccaggtggttgtgatgaattctgttgggaacaattgtactatgaactttaagaataacaatggaaacaatattggtctgctggggtttaaggcagatacggttgttgcttccacttggtattatacccatatgagagaccatacaaacagcaacggctgcttctggaatttcattagtgaggaacatggatggcaggagaagtaa

SEQ ID NO: 66 - DNA, 3759 bp, Artificial Sequence
  mat_peptide (1)...(3756): BoNT/E, X. laevis-modified 2
atgccaaaaattaattcttttaattataatgatccagtgaatgatagaacaattctgtatattaaaccaggaggatgtcaggaattttataaatcttttaatattatgaaaaatatttggattattccagaaagaaatgtgattggaacaacaccacaggattttcatccaccaacatctctgaaaaatggagattcttcttattatgatccaaattatctgcagtctgatgaagaaaaagatagatttctgaaaattgtgacaaaaatttttaatagaattaataataatctgtctggaggaattctgctggaagaactgtctaaagctaatccatatctgggaaatgataatacaccagataatcagtttcatattggagatgcttctgctgtggaaattaaattttctaatggatctcaggatattctgctgccaaatgtgattattatgggagctgaaccagatctgtttgaaacaaattcttctaatatttctctgagaaataattatatgccatctaatcatggatttggatctattgctattgtgacattttctccagaatattcttttagatttaatgataattctatgaatgaatttattcaggatccagctctgacactgatgcatgaactgattcattctctgcatggactgtatggagctaaaggaattacaacaaaatatacaattacacagaaacagaatccactgattacaaatattagaggaacaaatattgaagaatttctgacatttggaggaacagatctgaatattattacatctgctcagtctaatgatatttatacaaatctgctggctgattataaaaaaattgcttctaaactgtctaaagtgcaggtgtctaatccactgctgaatccatataaagatgtgtttgaagctaaatatggactggataaagatgcttctggaatttattctgtgaatattaataaatttaatgatatttttaaaaaactgtattcttttacagaatttgatctggctacaaaatttcaggtgaaatgtagacagacatatattggacagtataaatattttaaactgtctaatctgctgaatgattctatttataatatttctgaaggatataatattaataatctgaaagtgaattttagaggacagaatgctaatctgaatccaagaattattacaccaattacaggaagaggactggtgaaaaaaattattagattttgtaaaaatattgtgtctgtgaaaggaattagaaaatctatttgtattgaaattaataatggagaactgttttttgtggcttctgaaaattcttataatgatgataatattaatacaccaaaagaaattgatgatacagtgacatctaataataattatgaaaatgatctggatcaggtgattctgaattttaattctgaatctgctccaggactgtctgatgaaaaactgaatctgacaattcagaatgatgcttatattccaaaatatgattctaatggaacatctgatattgaacagcatgatgtgaatgaactgaatgtgtttttttatctggatgctcagaaagtgccagaaggagaaaataatgtgaatctgacatcttctattgatacagctctgctggaacagccaaaaatttatacatttttttcttctgaatttattaataatgtgaataaaccagtgcaggctgctctgtttgtgtcttggattcagcaggtgctggtggattttacaacagaagctaatcagaaatctacagtggataaaattgctgatatttctattgtggtgccatatattggactggctctgaatattggaaatgaagctcagaaaggaaattttaaagatgctctggaactgctgggagctggaattctgctggaatttgaaccagaactgctgattccaacaattctggtgtttacaattaaatcttttctgggatcttctgataataaaaataaagtgattaaagctattaataatgctctgaaagaaagagatgaaaaatggaaagaagtgtattcttttattgtgtctaattggatgacaaaaattaatacacagtttaataaaagaaaagaacagatgtatcaggctctgcagaatcaggtgaatgctattaaaacaattattgaatctaaatataattcttatacactggaagaaaaaaatgaactgacaaataaatatgatattaaacagattgaaaatgaactgaatcagaaagtgtctattgctatgaataatattgatagatttctgacagaatcttctatttcttatctgatgaaactgattaatgaagtgaaaattaataaactgagagaatatgatgaaaatgtgaaaacatatctgctgaattatattattcagcatggatctattctgggagaatctcagcaggaactgaattctatggtgacagatacactgaataattctattccatttaaactgtcttcttatacagatgataaaattctgatttcttattttaataaattttttaaaagaattaaatcttcttctgtgctgaatatgagatataaaaatgataaatatgtggatacatctggatatgattctaatattaatattaatggagatgtgtataaatatccaacaaataaaaatcagtttggaatttataatgataaactgtctgaagtgaatatttctcagaatgattatattatttatgataataaatataaaaatttttctatttctttttgggtgagaattccaaattatgataataaaattgtgaatgtgaataatgaatatacaattattaattgtatgagagataataattctggatggaaagtgtctctgaatcataatgaaattatttggacactgcaggataatgctggaattaatcagaaactggcttttaattatggaaatgctaatggaatttctgattatattaataaatggatttttgtgacaattacaaatgatagactgggagattctaaactgtatattaatggaaatctgattgatcagaaatctattctgaatctgggaaatattcatgtgtctgataatattctgtttaaaattgtgaattgttcttatacaagatatattggaattagatattttaatatttttgataaagaactggatgaaacagaaattcagacactgtattctaatgaaccaaatacaaatattctgaaagatttttggggaaattatctgctgtatgataaagaatattatctgctgaatgtgctgaaaccaaataattttattgatagaagaaaagattctacactgtctattaataatattagatctacaattctgctggctaatagactgtattctggaattaaagtgaaaattcagagagtgaataattcttctacaaatgataatctggtgagaaaaaatgatcaggtgtatattaattttgtggcttctaaaacacatctgtttccactgtatgctgatacagctacaacaaataaagaaaaaacaattaaaatttcttcttctggaaatagatttaatcaggtggtggtgatgaattctgtgggaaataattgtacaatgaattttaaaaataataatggaaataatattggactgctgggatttaaagctgatacagtggtggcttctacatggtattatacacatatgagagatcatacaaattctaatggatgtttttggaattttatttctgaagaacatggatggcaggaaaaataa

SEQ ID NO: 67 - DNA, 3759 bp, Artificial Sequence
  mat_peptide (1)...(3756): BoNT/E, X. laevis-modified 3
atgcctaaaatcaatagttttaactacaatgatcctgttaatgaccgcacaatcctgtacatcaaacccgggggatgtcaagaattttacaaaagtttcaatattatgaagaatatctggatcattcccgaaagaaatgtgattggtacaacccctcaggactttcacccacccacctctctgaaaaatggcgactcttcatactatgaccctaactaccttcaaagtgatgaggaaaaggatcgattcttgaaaatcgttacaaaaatttttaacaggataaataacaatctgagcggtgggatccttctagaggaacttagtaaggccaacccctacctgggcaacgacaacacaccagataatcagttccacattggagatgcttcagcagtggagatcaagttctctaatggctctcaggatatactgcttccaaacgttattataatgggagccgagcctgatttatttgagaccaattctagtaacattagcttgagaaataactatatgccaagcaaccatggatttggctctatcgccatcgtgacattctccccagaatactcattcaggtttaatgataactctatgaatgagttcattcaagaccctgctctgactctgatgcacgaactaatacactctctgcacgggttgtacggagctaagggaattaccactaaatatactataacacaaaaacagaatccattgattaccaacatccgcggtaccaatatagaggaatttttaactttcggcggtaccgatctgaacataatcacaagcgctcaatccaatgacatctacacaaatcttttggctgattacaaaaagattgcaagtaaattatcaaaggtgcaggtctccaatccactgcttaacccttataaggatgtctttgaggctaaatatggcctggacaaagacgcttccggtatctattccgtgaatataaacaagtttaacgatatatttaaaaagctatactctttcaccgagtttgatctggccactaagttccaggtgaaatgcaggcaaacatatattgggcagtataaatatttcaagttgtctaatcttctgaatgatagtatttataacatctcagaagggtataacatcaataacctaaaagtgaattttcgcggacagaatgccaacctcaacccacgaatcattactcctatcacaggccgggggcttgttaagaaaattatcaggttttgtaagaatatcgtcagtgtaaaaggaattagaaaaagtatatgcattgaaatcaacaatggtgaactcttctttgtggctagtgaaaatagctacaacgacgataatataaatactccaaaagagatagatgacactgtgacatcaaataacaattatgagaacgatttggaccaggttatcctcaactttaattctgaatcagcaccaggactttctgacgagaagctcaacctgactattcaaaatgatgcatacattccaaagtatgattcaaacggcacaagcgacatcgaacagcatgacgtaaatgagttaaatgtgttcttttatcttgatgcccagaaggtgccagaaggagaaaacaatgtaaatttgacatcttccattgatacagccttgttagagcagcctaagatttatacattctttagttctgaatttattaataacgtgaacaagccagtgcaggccgctttgtttgtatcttggatccaacaggtgcttgttgactttactacagaagcaaatcagaaaagcacagtcgataagatcgctgatatttccattgtagttccatacattggactggctctaaatatcggcaacgaagcacagaaggggaacttcaaagacgccttggagctattaggggctggtatactgctcgaattcgaacccgagctgcttatccccactatcttagtgtttaccattaagagtttcctgggtagctcagacaataagaacaaagttataaaggctattaataacgccttgaaagagagagatgaaaagtggaaggaggtatattcctttattgtgtctaattggatgactaaaataaatacccagtttaataaacgcaaggagcaaatgtaccaggctctgcaaaatcaggttaacgcaataaaaacaattatagaatcaaaatataactcttacaccctggaagagaaaaacgagcttactaataaatacgacattaaacagattgagaatgagctgaaccaaaaagtgagcattgctatgaataacattgatcggttcctgaccgaaagcagtatcagctaccttatgaaactcatcaatgaagtaaaaatcaataagctccgtgaatacgatgagaacgtcaaaacctatttactaaattacataattcaacatggatcaattttaggagaatctcagcaagagttaaattccatggtcactgacaccctcaataactctattcctttcaaattaagctcttatactgacgataagatcctgatatcatacttcaacaagtttttcaaacgaattaagagctcctctgtgctaaatatgcgttataaaaacgacaagtatgttgacacatctggatacgatagtaatatcaatataaatggagatgtttacaaatatccaacaaataagaaccagttcggtatctataacgacaaattgagcgaagtcaacattagtcaaaacgattacattatatacgacaacaagtacaagaacttctccattagcttttgggtcagaatacccaactacgataacaaaattgtcaacgttaacaatgaatacaccatcattaactgtatgagagataataacagcggctggaaagtgtccctcaaccacaacgagatcatttggacacttcaggataacgcaggaatcaatcagaagctggcatttaactatggcaatgctaacggcatttctgattatatcaacaaatggatctttgtgactataacaaacgatagattgggcgacagtaagctttatataaatggaaatctgatagatcaaaagtctatcctcaacttggggaatatccatgtatccgacaatattctctttaagattgtgaactgctcttatactagatacattggtatccgatacttcaacatttttgataaggagcttgacgaaacagagatacagaccctatactccaacgaaccaaatacaaatattctgaaggatttctggggaaactatctgttgtacgacaaggagtattacctactgaacgtcctgaagcctaataactttattgaccgtcggaaggattcaacactttcaatcaataacatcagatcaactatactccttgcaaatcggctgtatagcgggatcaaagttaaaatccagcgtgtgaataactcatccacaaacgacaatctcgttaggaagaacgatcaggtgtatattaattttgttgcatctaagactcatctgttccccctctatgcagacaccgcaactaccaataaggaaaaaacaattaaaataagttccagcgggaacaggtttaaccaagtagttgtcatgaactccgtaggtaataactgtacaatgaatttcaagaataacaatggaaataacataggtctcttgggattcaaagccgatactgtcgtagcttccacctggtattatacacatatgagggatcacacaaattctaatgggtgcttttggaattttattagcgaggaacatggctggcaggagaaataa

SEQ ID NO: 68 - DNA, 3759 bp, Artificial Sequence
  mat_peptide (1)...(3756): BoNT/E, X. tropicalis-modified 1
atgccaaagatcaatagtttcaattacaacgatcctgttaatgatcgtacaatactgtacattaagcctgggggctgccaggagttctataaatcgtttaatataatgaaaaacatatggattatccctgagaggaatgtgattggaactacgccacaggatttccacccacctacgtctttgaagaacggtgatagtagctattacgatcctaattaccttcagtctgatgaggaaaaagatagattcctgaagatagtcacaaaaatcttcaacagaattaataacaatctctctgggggaatattgctagaagagcttagcaaagcaaatccatacctggggaatgataatacaccagataatcaattccatatcggagatgctagcgcagtagaaattaagttcagcaacggttctcaggatattttgttaccgaatgtaatcataatgggggcagagccagatctctttgaaaccaattccagtaatatctcacttaggaacaattatatgcccagcaaccatggattcggatccatcgccattgtcacattttcacccgaatatagttttagatttaatgataattcgatgaacgagttcatccaagatccagcactgactttaatgcacgagctcattcattcattacatggtctctatggcgccaaggggattaccacaaagtataccatcacccagaaacagaaccccttgattaccaacatccgagggactaatattgaggaattcctgacgtttggtggaacggacttaaacattataacgtctgcccagagtaacgacatttacactaaccttttggctgactataaaaagattgcgtcaaagctctcaaaggtacaggtatcaaaccctcttctaaatccatacaaagatgtttttgaagctaagtatggcttggacaaggacgcttccggaatctactcagtgaatataaacaaatttaatgatatcttcaagaaactatactcatttacagaattcgatctggcaactaagttccaagtcaaatgtcgtcagacatatatcggtcagtataaatactttaaactttctaacctcttaaacgactccatctacaacatatctgagggctacaacataaacaatcttaaggttaatttccgcggccaaaatgccaatttgaaccccagaatcattacaccaatcacaggccgtggactggtgaaaaagattatccgattctgcaagaacattgtgtccgttaagggcataaggaaaagcatctgtattgagataaacaatggcgagctatttttcgttgcttctgagaactcgtacaatgacgataatatcaacacccccaaagagatagacgatacagtaaccagtaataacaattacgaaaatgatcttgatcaggtcattttaaattttaacagcgaaagtgcaccgggcctatctgacgagaagctgaatttaaccatccaaaacgatgcctacattcctaaatatgattccaacggaactagtgatattgagcagcacgacgttaacgaactgaatgtctttttctatcttgatgcccagaaggtacctgaaggagagaataacgttaatctaactagctccattgacactgccctgttggaacagcccaagatttacacattcttttcctctgaattcattaacaatgtgaataagcccgtccaggctgccctatttgtttcctggatacagcaagtcctggtagactttactacggaggcaaaccagaagagcaccgtcgacaagatagctgatatcagcatagtggtcccttatatcggcctggccctcaatatcgggaacgaagcccagaaagggaactttaaggacgcgcttgagctgcttggagctgggatcttgctggaattcgaaccagagttgctcattccgacaatcctggtttttactattaagagtttcctcggatcatccgacaataagaataaggtgataaaagcaataaacaatgctcttaaggaacgcgatgagaagtggaaagaggtgtattcttttatagtgagcaactggatgactaaaataaacacccagtttaacaaacggaaagagcaaatgtatcaggctctgcaaaaccaggtgaatgcgatcaaaaccattatcgaaagtaaatacaattcgtatacattagaagagaaaaacgaacttacaaataaatacgacattaaacagatcgaaaatgaactgaaccagaaggtgagcattgcaatgaataacattgaccgcttccttactgaaagctccatatcctatctgatgaagctgatcaatgaggtaaagatcaacaaactgagagaatacgacgagaacgttaaaacatatctacttaactacattatacaacatggcagcatcctgggtgaaagtcaacaggagttgaacagtatggtcacagacacacttaataactctatcccctttaaactctcatcttataccgacgataagatactaattagttatttcaataaatttttcaaaagaatcaagtcttccagcgtgctcaacatgaggtataaaaatgataaatacgttgatacaagcggatacgatagcaacattaacatcaatggggatgtttacaagtatccaacaaataaaaaccagtttggtatatataatgacaagttgagcgaagtgaatatttcccaaaatgactatataatctacgataataaatataaaaacttttctatatcgttttgggtgagaatcccaaattacgataataagatcgtgaacgtgaataacgaatatactataattaactgtatgagggataataactccggatggaaagtgagtttaaaccacaatgaaattatctggacactccaggataatgccggaattaaccaaaaattggcatttaattacggaaacgctaatggtatctctgactatattaataaatggatctttgtgaccattactaacgaccggctgggagactctaaattgtacattaacgggaatctgattgatcaaaaatccatccttaacctcggcaatattcacgtgtcagataatatcctgtttaagatcgtaaattgcagttacactcggtacattggtattcgctattttaatatttttgacaaagagttggacgaaaccgaaatccagaccctgtatagcaacgagccaaacactaatattctgaaggacttctggggcaactacctgctctatgacaaggagtactatttgctgaacgtcttaaagcctaataacttcatcgatcgaagaaaagattcaacactgtctataaacaatattcggtctaccatcctacttgcaaacaggctgtattccgggattaaagttaagattcagcgagtaaataactcatctaccaacgataacctggtaaggaagaatgatcaggtttatattaattttgtggcttcaaagactcacctcttccctctgtacgctgacaccgccacaactaacaaagagaagaccattaagatatcatcttccgggaaccgtttcaatcaagtggttgtgatgaatagcgttggtaacaattgcactatgaattttaagaataacaatggaaacaacattggtctgcttggatttaaggcagacactgtcgtggcaagtacgtggtactacacacatatgcgcgaccacacaaactccaacggctgtttctggaactttatttctgaggaacatggttggcaagagaagtaa

SEQ ID NO: 69 - DNA, 3759 bp, Artificial Sequence
  mat_peptide (1)...(3756): BoNT/E, X. tropicalis-modified 2
atgccaaagattaattcttttaattacaatgatccagtgaatgatagaacaattctgtacattaagccaggaggatgtcaggaattttacaagtcttttaatattatgaagaatatttggattattccagaaagaaatgtgattggaacaacaccacaggattttcatccaccaacatctctgaagaatggagattcttcttactacgatccaaattacctgcagtctgatgaagaaaaggatagatttctgaagattgtgacaaagatttttaatagaattaataataatctgtctggaggaattctgctggaagaactgtctaaggcaaatccatacctgggaaatgataatacaccagataatcagtttcatattggagatgcatctgcagtggaaattaagttttctaatggatctcaggatattctgctgccaaatgtgattattatgggagcagaaccagatctgtttgaaacaaattcttctaatatttctctgagaaataattacatgccatctaatcatggatttggatctattgcaattgtgacattttctccagaatactcttttagatttaatgataattctatgaatgaatttattcaggatccagcactgacactgatgcatgaactgattcattctctgcatggactgtacggagcaaagggaattacaacaaagtacacaattacacagaagcagaatccactgattacaaatattagaggaacaaatattgaagaatttctgacatttggaggaacagatctgaatattattacatctgcacagtctaatgatatttacacaaatctgctggcagattacaagaagattgcatctaagctgtctaaggtgcaggtgtctaatccactgctgaatccatacaaggatgtgtttgaagcaaagtacggactggataaggatgcatctggaatttactctgtgaatattaataagtttaatgatatttttaagaagctgtactcttttacagaatttgatctggcaacaaagtttcaggtgaagtgtagacagacatacattggacagtacaagtactttaagctgtctaatctgctgaatgattctatttacaatatttctgaaggatacaatattaataatctgaaggtgaattttagaggacagaatgcaaatctgaatccaagaattattacaccaattacaggaagaggactggtgaagaagattattagattttgtaagaatattgtgtctgtgaagggaattagaaagtctatttgtattgaaattaataatggagaactgttttttgtggcatctgaaaattcttacaatgatgataatattaatacaccaaaggaaattgatgatacagtgacatctaataataattacgaaaatgatctggatcaggtgattctgaattttaattctgaatctgcaccaggactgtctgatgaaaagctgaatctgacaattcagaatgatgcatacattccaaagtacgattctaatggaacatctgatattgaacagcatgatgtgaatgaactgaatgtgtttttttacctggatgcacagaaggtgccagaaggagaaaataatgtgaatctgacatcttctattgatacagcactgctggaacagccaaagatttacacatttttttcttctgaatttattaataatgtgaataagccagtgcaggcagcactgtttgtgtcttggattcagcaggtgctggtggattttacaacagaagcaaatcagaagtctacagtggataagattgcagatatttctattgtggtgccatacattggactggcactgaatattggaaatgaagcacagaagggaaattttaaggatgcactggaactgctgggagcaggaattctgctggaatttgaaccagaactgctgattccaacaattctggtgtttacaattaagtcttttctgggatcttctgataataagaataaggtgattaaggcaattaataatgcactgaaggaaagagatgaaaagtggaaggaagtgtactcttttattgtgtctaattggatgacaaagattaatacacagtttaataagagaaaggaacagatgtaccaggcactgcagaatcaggtgaatgcaattaagacaattattgaatctaagtacaattcttacacactggaagaaaagaatgaactgacaaataagtacgatattaagcagattgaaaatgaactgaatcagaaggtgtctattgcaatgaataatattgatagatttctgacagaatcttctatttcttacctgatgaagctgattaatgaagtgaagattaataagctgagagaatacgatgaaaatgtgaagacatacctgctgaattacattattcagcatggatctattctgggagaatctcagcaggaactgaattctatggtgacagatacactgaataattctattccatttaagctgtcttcttacacagatgataagattctgatttcttactttaataagttttttaagagaattaagtcttcttctgtgctgaatatgagatacaagaatgataagtacgtggatacatctggatacgattctaatattaatattaatggagatgtgtacaagtacccaacaaataagaatcagtttggaatttacaatgataagctgtctgaagtgaatatttctcagaatgattacattatttacgataataagtacaagaatttttctatttctttttgggtgagaattccaaattacgataataagattgtgaatgtgaataatgaatacacaattattaattgtatgagagataataattctggatggaaggtgtctctgaatcataatgaaattatttggacactgcaggataatgcaggaattaatcagaagctggcatttaattacggaaatgcaaatggaatttctgattacattaataagtggatttttgtgacaattacaaatgatagactgggagattctaagctgtacattaatggaaatctgattgatcagaagtctattctgaatctgggaaatattcatgtgtctgataatattctgtttaagattgtgaattgttcttacacaagatacattggaattagatactttaatatttttgataaggaactggatgaaacagaaattcagacactgtactctaatgaaccaaatacaaatattctgaaggatttttggggaaattacctgctgtacgataaggaatactacctgctgaatgtgctgaagccaaataattttattgatagaagaaaggattctacactgtctattaataatattagatctacaattctgctggcaaatagactgtactctggaattaaggtgaagattcagagagtgaataattcttctacaaatgataatctggtgagaaagaatgatcaggtgtacattaattttgtggcatctaagacacatctgtttccactgtacgcagatacagcaacaacaaataaggaaaagacaattaagatttcttcttctggaaatagatttaatcaggtggtggtgatgaattctgtgggaaataattgtacaatgaattttaagaataataatggaaataatattggactgctgggatttaaggcagatacagtggtggcatctacatggtactacacacatatgagagatcatacaaattctaatggatgtttttggaattttatttctgaagaacatggatggcaggaaaagtaa

SEQ ID NO: 70 - DNA, 3759 bp, Artificial Sequence
  mat_peptide (1)...(3756): BoNT/E, X. tropicalis-modified 3
atgcccaagattaatagcttcaactacaacgatccagtgaatgacagaacaatactgtatattaagcctggcggatgtcaggagttctataagtccttcaacattatgaaaaacatctggattatccccgaacggaatgtcatagggactacaccccaggacttccaccctccaactagtctcaaaaatggggattcttcctactatgatccaaactatcttcagtccgacgaggaaaaggatcgttttctgaaaatcgttaccaagatatttaacagaataaacaataacctgtctggcggaattttgctggaggaattgagtaaagctaacccataccttggaaatgacaataccccagacaaccaatttcatattggagatgcttctgccgtcgaaatcaagttttctaacggttcacaagatatcctactgcctaatgttataattatgggagctgaacctgatttattcgaaacaaacagttctaacatatccctcaggaataactatatgcccagtaaccacggcttcgggtcaattgccatcgtcacattttccccagaatatagttttcggtttaacgacaactccatgaatgaattcattcaggacccagcattgacacttatgcacgaactgattcattcactgcatggcctttacggagcaaagggaatcactaccaaatatactattacccagaaacagaacccactcattacaaatatcagaggcaccaacattgaagagtttctaactttcggagggactgatctcaatattatcacaagcgctcagtccaacgacatctacacaaacttactggctgattacaaaaagatagcttcaaaactgagcaaagtgcaggtgtccaacccacttctgaatccttataaagatgttttcgaagcaaagtacggcctggataaagatgcttcagggatatattctgtaaacattaataagttcaacgacatcttcaaaaagctttattctttcacagaatttgatttagcaaccaagtttcaagtcaagtgccgtcaaacctatattgggcagtacaagtattttaaactttctaatttgcttaatgacagcatatataatatttcagaaggttataacataaacaatttaaaagtgaatttccgcggacagaatgccaatctgaatcctaggattatcaccccaataaccggaagggggctggttaagaaaattatccgtttttgcaagaacattgtaagcgtcaaaggtatccggaaatctatttgcatcgagattaataacggggagttgttctttgttgcatctgagaatagttataatgacgataatatcaacactcccaaggagattgatgacactgttacttctaacaataactatgagaatgatttggaccaagtcatacttaattttaattccgaatcagcccccggcctctccgatgagaagctgaatttaacaatacagaatgatgcatacatcccaaagtatgacagcaatggtacatctgatatcgaacagcacgatgttaacgaactgaatgtattcttttacctggacgcacagaaagtgcccgagggagagaataacgtgaacctgacaagcagtattgatacagcccttttggagcagccaaaaatatatacttttttcagctctgagttcatcaataacgtaaataagcctgtgcaagctgcactatttgtgagctggattcagcaagtcttagtggattttacaaccgaagctaaccagaaatctacagttgacaaaatagcagatatttctattgtagttccatatattggcttggcattgaacatcggaaacgaagcacaaaagggcaacttcaaggatgccctcgagctgttgggagctggtattttgctagaatttgaaccagagctcctgatccctacaatcctggtatttacaattaaaagctttttaggtagttctgacaacaagaacaaagtaatcaaggctatcaataacgctcttaaagaaagagatgagaagtggaaagaagtgtattcatttatcgtgagcaattggatgactaagattaatacccaatttaataagcgaaaggagcagatgtaccaggccctgcagaaccaggtaaatgccataaagacaattatcgagtctaaatacaactcctacacccttgaggaaaaaaacgagctcacaaataagtacgatataaagcagatagagaatgagcttaaccaaaaagtgtccattgccatgaacaatatagatcgcttcctgactgagagttccatcagttacttgatgaaactaatcaatgaagtcaaaatcaataaactgagagaatatgacgagaacgtcaaaacttacctactgaattacataattcagcatgggtctattttgggtgagagtcaacaggaactgaattctatggttactgacacactaaacaatagcattcctttcaaactctcaagttatacagatgacaagattctgatttcttactttaataaatttttcaagcgcattaaatcctctagcgtccttaacatgagatataaaaacgataagtacgtggacacaagtggctacgattccaatattaacattaatggggacgtgtacaaataccccaccaataagaatcagtttggaatttataatgataaacttagcgaagtgaatatatcacaaaatgattacatcatttacgacaacaaatataaaaatttttccatctcattctgggttaggattcctaactacgataataagattgtgaacgtcaacaatgagtatacaatcattaactgcatgagagacaataactccgggtggaaggtttccttgaaccataatgagatcatatggaccctccaggataatgctggaataaaccaaaagctggcattcaactatgggaacgcaaatggtatctcagattacatcaacaagtggatattcgttactattactaatgatcgactgggtgattctaagctttacataaacggcaatctgatcgaccaaaagtctattctgaatctggggaatattcacgtctcagacaatatactctttaagatcgtaaactgttcttataccagatacatcggcatccgatactttaatattttcgacaaggaactggacgagaccgaaattcagactctatatagcaacgagcctaacacaaatatcttaaaggacttttggggaaactatctcctttacgataaagaatactatctacttaatgtgttgaaaccaaacaatttcattgataggcggaaggatagcacattgagcataaacaatatccgctcaacaatattactagcaaaccgattatattctggaattaaggttaagatccagagggtgaataactccagtacaaacgataacctcgtacgtaagaacgaccaggtgtatatcaacttcgtggcctctaaaacacatctgtttccactttacgcagacactgccacaactaataaggagaagacaatcaaaatcagtagctcaggtaacaggtttaaccaggtagtggttatgaacagcgtgggaaacaattgtaccatgaacttcaaaaacaataacggcaataatataggcctcctcggttttaaagccgacaccgttgtggcaagtacatggtactacacccacatgcgcgatcacactaattctaatggttgtttttggaatttcatcagcgaagagcatggatggcaagaaaaataa

SEQ ID NO: 71 - DNA, 3759 bp, Artificial Sequence
  mat_peptide (1)...(3756): BoNT/E, G. gallus-modified 1
atgcccaagatcaatagttttaactacaacgacccagtgaacgaccgcacaatactgtacataaagccaggtggctgtcaggaattttataaatcattcaacatcatgaaaaatatctggatcattcccgaaagaaatgtcatcggaacaactccacaagacttccatccacccacttcactcaaaaacggagatagctcatactatgatcctaactatctgcagtcagacgaggaaaaggaccggtttctgaaaatcgtaactaagattttcaatcggatcaacaataacttatcgggcggaatcctgttggaggaattgtcaaaggcaaacccgtacctaggaaacgacaacacaccagacaaccaattccacattggggacgcctctgcggtcgagattaaatttagcaacggctcccaggatatcctgttacctaatgttatcattatgggggccgaacccgatctgtttgagacaaacagctcaaacatatctctgagaaacaattacatgccaagcaatcacggctttgggtctatcgcaatcgtgacatttagccccgaatatagtttcaggttcaacgacaattctatgaacgaattcattcaagatcccgcccttaccttaatgcatgagctaatccacagtcttcacggcctgtatggggctaagggaataacgacaaagtacacaatcacccagaaacagaatccgctgatcacgaacatccgcggaacaaatattgaagagttccttaccttcggagggacagacctgaacattataacgagcgcacagtctaacgacatttacactaacttactggctgattataaaaagattgcttcaaagctgagtaaggtccaggtcagtaatcccctcctgaacccctataaggacgtgtttgaggccaagtatggcttggataaagacgcctccggaatctacagtgtcaacatcaataaatttaacgatatctttaagaaactgtactctttcactgagttcgatctggcaacaaaattccaggtcaagtgtagacaaacctatattggccagtataaatactttaaactaagtaacctcctgaacgactctatctacaacattagcgagggatacaatattaataacctgaaagtgaattttcgaggacagaatgctaaccttaacccgaggataatcaccccaatcactggacggggcctggttaagaaaatcataaggttctgcaagaacattgttagtgttaaaggaattcgtaagtcgatctgcattgaaattaacaatggtgagctcttctttgtggcatctgaaaactcctacaacgatgacaacattaacacacctaaagaaatcgatgacactgtgactagcaataacaattacgagaacgaccttgaccaggtcattctcaacttcaactccgagtctgcccccgggctgtccgatgaaaagctgaacctgacgatacaaaatgacgcttacatcccaaaatacgatagtaacgggacaagcgacattgagcagcatgatgtgaatgagctcaatgtttttttctatctggacgcccagaaggtgccagagggggaaaacaatgtcaacctcacttccagcatagataccgcgctgcttgaacagccaaagatctacacctttttctccagtgagttcatcaataacgtaaataaacctgtccaggccgcattgttcgtgtcgtggattcaacaggtcctcgtagatttcaccacggaggcaaaccagaagtcaacagtggataagattgcagacatctctatcgtagtgccctacatcgggctggccttgaacatcggcaatgaggcacagaagggcaactttaaggacgctctggagcttctgggtgcgggaattctgttagaatttgaaccggagctcctaatccctacaatcctggtattcacaatcaaatcctttttgggttccagtgacaataagaataaagtgataaaggctattaacaatgccctgaaagaacgcgatgagaagtggaaggaggtttactcatttatcgtttctaactggatgaccaaaataaacacccaatttaataaaagaaaagagcagatgtatcaggcgctgcaaaatcaggtgaacgctattaaaacaattatagaatccaagtacaattcttacactctggaagagaaaaatgagcttactaataagtacgatataaagcagatcgagaacgagttgaatcagaaggttagcattgcgatgaataacattgacaggttcctcacggagagctcgattagctatctgatgaagcttattaacgaggttaaaattaacaagcttagagaatacgatgaaaacgtgaagacatatttgttaaattacatcatacaacatggttccattctaggggaatcacagcaagaactgaattctatggtcacggacacgttaaacaattcgattcctttcaagctgagctcctacaccgatgacaagattcttatctcctactttaataaatttttcaagcggatcaaaagttccagcgttctgaacatgcgctataaaaatgataaatacgttgatacctcaggctacgattctaatatcaatatcaatggcgacgtgtacaaatatccgactaataaaaaccagtttggtatttataatgataagctgagcgaggttaatatctcacagaacgattacatcatatatgataacaagtacaagaacttttcaatctccttttgggtgcgtatccctaattacgacaataagatagtgaatgtgaacaatgagtataccatcattaactgcatgcgagacaacaattcaggctggaaagtgtccctgaatcacaacgagattatctggacacttcaagataacgcagggatcaatcagaaactggctttcaactatgggaatgccaatggtatttccgattatataaacaagtggatattcgtaaccattacgaacgatagactcggtgactcgaaactttacataaatggtaacttgatagatcagaagagcatactcaacttgggaaacatccatgtgtccgataacatactgtttaagatcgtgaattgcagctacactaggtatattggtattaggtatttcaatatcttcgacaaggagctggacgaaaccgaaatccagacgctctatagcaacgaacccaacaccaacatcctcaaagatttctggggaaattacctcttgtatgataaggagtactatctccttaatgtgctcaagcctaacaatttcatcgaccgacggaaagacagtactttgagcattaacaatattagaagcaccatattgctcgctaataggctatactccggaatcaaagtcaagatccagcgcgtgaacaattctagcaccaatgacaacctggtgcggaagaacgatcaggtttacatcaacttcgtagcatccaagactcacctgttccctttatacgctgatactgctacaaccaacaaagagaaaaccattaagatcagcagttctggcaaccgcttcaaccaagtggtagtgatgaacagcgtcgggaataactgcaccatgaacttcaagaacaataacggcaataacatcgggctgttgggctttaaagccgacaccgtggtggcctcgacttggtattacacacatatgcgtgatcacacaaactctaatggctgtttttggaactttattagcgaagagcacggctggcaggaaaagtaa

SEQ ID NO: 72 - DNA, 3759 bp, Artificial Sequence
  mat_peptide (1)...(3756): BoNT/E, G. gallus-modified 2
atgcccaagatcaacagcttcaactacaacgatcccgtgaacgatagaaccatcctgtacatcaagcccggcggctgccaggagttctacaagagcttcaacatcatgaagaacatctggatcatccccgagagaaacgtgatcggcaccaccccccaggatttccacccccccaccagcctgaagaacggcgatagcagctactacgatcccaactacctgcagagcgatgaggagaaggatagattcctgaagatcgtgaccaagatcttcaacagaatcaacaacaacctgagcggcggcatcctgctggaggagctgagcaaggccaacccctacctgggcaacgataacacccccgataaccagttccacatcggcgatgccagcgccgtggagatcaagttcagcaacggcagccaggatatcctgctgcccaacgtgatcatcatgggcgccgagcccgatctgttcgagaccaacagcagcaacatcagcctgagaaacaactacatgcccagcaaccacggcttcggcagcatcgccatcgtgaccttcagccccgagtacagcttcagattcaacgataacagcatgaacgagttcatccaggatcccgccctgaccctgatgcacgagctgatccacagcctgcacggcctgtacggcgccaagggcatcaccaccaagtacaccatcacccagaagcagaaccccctgatcaccaacatcagaggcaccaacatcgaggagttcctgaccttcggcggcaccgatctgaacatcatcaccagcgcccagagcaacgatatctacaccaacctgctggccgattacaagaagatcgccagcaagctgagcaaggtgcaggtgagcaaccccctgctgaacccctacaaggatgtgttcgaggccaagtacggcctggataaggatgccagcggcatctacagcgtgaacatcaacaagttcaacgatatcttcaagaagctgtacagcttcaccgagttcgatctggccaccaagttccaggtgaagtgcagacagacctacatcggccagtacaagtacttcaagctgagcaacctgctgaacgatagcatctacaacatcagcgagggctacaacatcaacaacctgaaggtgaacttcagaggccagaacgccaacctgaaccccagaatcatcacccccatcaccggcagaggcctggtgaagaagatcatcagattctgcaagaacatcgtgagcgtgaagggcatcagaaagagcatctgcatcgagatcaacaacggcgagctgttcttcgtggccagcgagaacagctacaacgatgataacatcaacacccccaaggagatcgatgataccgtgaccagcaacaacaactacgagaacgatctggatcaggtgatcctgaacttcaacagcgagagcgcccccggcctgagcgatgagaagctgaacctgaccatccagaacgatgcctacatccccaagtacgatagcaacggcaccagcgatatcgagcagcacgatgtgaacgagctgaacgtgttcttctacctggatgcccagaaggtgcccgagggcgagaacaacgtgaacctgaccagcagcatcgataccgccctgctggagcagcccaagatctacaccttcttcagcagcgagttcatcaacaacgtgaacaagcccgtgcaggccgccctgttcgtgagctggatccagcaggtgctggtggatttcaccaccgaggccaaccagaagagcaccgtggataagatcgccgatatcagcatcgtggtgccctacatcggcctggccctgaacatcggcaacgaggcccagaagggcaacttcaaggatgccctggagctgctgggcgccggcatcctgctggagttcgagcccgagctgctgatccccaccatcctggtgttcaccatcaagagcttcctgggcagcagcgataacaagaacaaggtgatcaaggccatcaacaacgccctgaaggagagagatgagaagtggaaggaggtgtacagcttcatcgtgagcaactggatgaccaagatcaacacccagttcaacaagagaaaggagcagatgtaccaggccctgcagaaccaggtgaacgccatcaagaccatcatcgagagcaagtacaacagctacaccctggaggagaagaacgagctgaccaacaagtacgatatcaagcagatcgagaacgagctgaaccagaaggtgagcatcgccatgaacaacatcgatagattcctgaccgagagcagcatcagctacctgatgaagctgatcaacgaggtgaagatcaacaagctgagagagtacgatgagaacgtgaagacctacctgctgaactacatcatccagcacggcagcatcctgggcgagagccagcaggagctgaacagcatggtgaccgataccctgaacaacagcatccccttcaagctgagcagctacaccgatgataagatcctgatcagctacttcaacaagttcttcaagagaatcaagagcagcagcgtgctgaacatgagatacaagaacgataagtacgtggataccagcggctacgatagcaacatcaacatcaacggcgatgtgtacaagtaccccaccaacaagaaccagttcggcatctacaacgataagctgagcgaggtgaacatcagccagaacgattacatcatctacgataacaagtacaagaacttcagcatcagcttctgggtgagaatccccaactacgataacaagatcgtgaacgtgaacaacgagtacaccatcatcaactgcatgagagataacaacagcggctggaaggtgagcctgaaccacaacgagatcatctggaccctgcaggataacgccggcatcaaccagaagctggccttcaactacggcaacgccaacggcatcagcgattacatcaacaagtggatcttcgtgaccatcaccaacgatagactgggcgatagcaagctgtacatcaacggcaacctgatcgatcagaagagcatcctgaacctgggcaacatccacgtgagcgataacatcctgttcaagatcgtgaactgcagctacaccagatacatcggcatcagatacttcaacatcttcgataaggagctggatgagaccgagatccagaccctgtacagcaacgagcccaacaccaacatcctgaaggatttctggggcaactacctgctgtacgataaggagtactacctgctgaacgtgctgaagcccaacaacttcatcgatagaagaaaggatagcaccctgagcatcaacaacatcagaagcaccatcctgctggccaacagactgtacagcggcatcaaggtgaagatccagagagtgaacaacagcagcaccaacgataacctggtgagaaagaacgatcaggtgtacatcaacttcgtggccagcaagacccacctgttccccctgtacgccgataccgccaccaccaacaaggagaagaccatcaagatcagcagcagcggcaacagattcaaccaggtggtggtgatgaacagcgtgggcaacaactgcaccatgaacttcaagaacaacaacggcaacaacatcggcctgctgggcttcaaggccgataccgtggtggccagcacctggtactacacccacatgagagatcacaccaacagcaacggctgcttctggaacttcatcagcgaggagcacggctggcaggagaagtaa

SEQ ID NO: 73 - DNA, 3759 bp, Artificial Sequence
  mat_peptide (1)...(3756): BoNT/E, G. gallus-modified 3
atgccgaagataaattcatttaattataacgacccagttaacgaccggaccatactttacatcaagccagggggctgccaagagttttacaaaagctttaacatcatgaagaatatttggattataccggaacgcaatgtgattggaactacgcctcaggatttccaccctccaacaagtctgaagaacggtgatagttcctactatgatcccaactaccttcagtctgacgaagagaaggataggttccttaagattgtgacgaaaattttcaatcgtatcaataacaatctcagcggcggtattctgttggaggaacttagcaaagccaatccgtacctcggtaacgataacactccggataaccagttccatatcggtgacgcgtctgcagtcgagatcaaattttcaaatggctctcaggacatactgctcccaaacgtgatcattatgggcgcagagcctgacctgtttgaaaccaattctagcaacatatccctccgcaataactacatgccctccaaccacggatttggaagtatcgctattgtgactttcagccctgagtatagctttcgatttaacgacaacagcatgaatgaatttattcaggaccccgctctgacactgatgcatgagctgatacatagccttcacggactgtatggggctaaggggattacgacaaaatataccatcacgcagaaacagaaccccctgatcaccaacatcagaggcactaatatcgaggaattccttacattcggcggaaccgacctgaatataattacatccgctcaaagcaacgatatctatacaaacctgcttgcagattacaaaaagatcgcatcaaaactgagcaaagtccaggttagtaaccccctgctcaatccatacaaagacgttttcgaggccaaatatggtctggacaaggatgccagcgggatttacagtgtgaacattaataaatttaacgacattttcaagaaactgtacagcttcacggagtttgacctggctactaagtttcaagttaagtgcagacagacgtacatcgggcaatacaagtacttcaaactcagcaacctgctcaacgacagtatctacaacattagtgagggatacaatatcaataacttgaaggtgaacttcaggggtcagaatgccaacctgaacccaagaattatcactcctattacggggcgcggattggttaagaaaatcattaggttctgcaagaacatcgtcagcgtgaaaggcattaggaagtctatctgtatcgagataaacaatggagaactgtttttcgtggcttccgaaaatagctacaacgacgataacattaatacccctaaagagatcgacgatacagtgacatcaaataacaattacgagaacgatctggaccaagttatcttgaactttaactccgaatctgcaccaggtctgagcgacgaaaagctgaatctgacgattcagaatgacgcgtacattcccaagtatgattccaatggaacctcagacatcgagcagcacgatgtgaacgaactgaacgtgttcttttacctggatgctcagaaagtgcctgaaggagaaaataacgtgaacctgacttcttccatagacaccgccttgctggagcagcccaaaatctataccttcttttctagtgaattcatcaataacgtgaacaagcccgtccaggcggctctctttgtgtcctggatccagcaagtgctggtggattttacaaccgaagctaaccagaaatcaactgtcgataagattgctgatatctcaatagtggttccttacattggcctggccctgaatatagggaacgaggctcagaagggcaactttaaagacgcactggagctgttgggagccgggatccttctggaatttgagccagagctgctcatacccaccattctggtgttcacaataaaatcatttctgggatccagcgataataaaaacaaagtcatcaaagcgattaacaatgccctcaaggagcgcgacgagaagtggaaggaggtgtactccttcattgtgagcaattggatgactaagatcaacactcagttcaataagcgaaaagaacaaatgtatcaggccctgcaaaaccaagtgaacgcaatcaagacaataatcgagtcaaaatataattcctacacactggaggaaaagaacgagttgaccaacaagtacgatattaagcaaattgagaacgagctcaaccagaaggtgagtattgccatgaacaatattgaccgctttttgaccgagtcctctatcagctacctgatgaagcttattaacgaagtgaagattaacaaactcagggaatatgacgagaacgtgaagacttatctcttgaattatataatccaacacgggtctatccttggagaaagtcagcaagaacttaatagcatggttaccgacactctgaataacagcattccattcaaactgagctcctataccgacgataaaatcctcatctcatatttcaacaaattctttaaacggatcaagagcagttctgtcctgaatatgcgttacaagaatgataaatacgtggacacgagcggctacgatagcaacatcaacatcaatggagacgtgtacaagtatccgacgaacaaaaatcagttcggcatttataatgataagctgagcgaggtgaatatctctcagaacgattacattatctacgataataaatacaagaatttctctatatccttctgggtgagaattcccaattatgataacaaaatcgttaacgtgaataacgagtacactataatcaactgtatgagggataacaattccggttggaaagtttctcttaaccacaacgagatcatatggacactgcaggataatgcaggcatcaaccagaagttggcattcaactacggcaatgcaaatgggattagcgactacatcaacaagtggattttcgtcaccatcaccaatgatcgtcttggcgactcaaaactgtatattaacggcaacttgatcgaccagaaaagcattctgaacctgggaaacatccacgtctcagacaatatcttgttcaaaatcgtcaactgctcttatactcggtatatcggcatcagatattttaatatttttgataaggaactcgatgaaacagagattcagacactgtatagcaacgaacctaacactaatatactcaaggacttttgggggaactaccttctgtatgataaagaatactatttgctgaatgtgctgaagccaaacaatttcatcgatcggcgcaaggactcaaccctgtctatcaataacattagatccaccatcctcctggctaaccggttgtattcagggattaaggtcaagatacagagagtgaacaatagttccacaaatgataacctggtgcggaagaacgaccaggtgtacataaacttcgttgcctccaagactcatctgttccccctctacgcagacaccgccactacaaataaggaaaaaacaatcaagatcagctccagcggcaataggttcaaccaggttgtcgtgatgaacagtgttggtaataactgcacaatgaactttaagaataacaatgggaacaatataggactgctgggctttaaggcggataccgtcgtggcctctacctggtactacacacatatgcgagatcacacaaatagtaatggttgtttctggaattttataagcgaggaacatgggtggcaggagaaataa

SEQ ID NO: 74 - DNA, 3759 bp, Artificial Sequence
  mat_peptide (1)...(3756): BoNT/E, M. musculus-modified 1
atgcccaaaataaacagctttaactataatgaccctgtgaacgatagaacaattctgtacatcaagccagggggatgccaggagttttataagagcttcaatattatgaaaaatatttggatcattcctgaacgcaacgtcatcgggaccacaccacaagatttccatcctccgacatctctgaagaatggcgactcatcctattacgacccaaactacctgcaatccgacgaagagaaagacagattcctgaaaattgtgaccaagatctttaatcgaataaacaataacctgtctggtggcatcctcctggaggaactgtcaaaggccaatccttacctggggaatgacaatacacccgataaccagttccacattggcgatgcctccgccgtcgaaataaagtttagcaacggaagccaagacattttgcttccaaacgttattatcatgggcgcagagccagatctgttcgagactaattcaagtaacatcagtctgcgaaacaattacatgccttccaaccacgggtttggcagtatcgctattgtgacgttttctcccgagtactcttttcgttttaatgacaacagcatgaacgagttcatccaggatcccgccctcactttgatgcacgagcttattcacagtctacatggactttatggcgctaagggtattacgaccaaatacacgatcacacaaaaacagaacccactcatcactaatatcagagggacaaatatcgaagagttcttaacgttcggcggaacggatcttaacatcattacctccgcacagtccaatgacatttatactaacctgctagcagactacaaaaagatcgcaagcaagctgagcaaagttcaggtctctaatccattgctgaatccctacaaagatgtgtttgaggctaagtatggtctagataaggatgcttccggtatctattctgtaaatattaacaaatttaacgacatcttcaaaaagctttacagcttcactgagtttgacctcgccacaaagttccaggtaaagtgtaggcagacatacataggccagtacaagtactttaaactgtccaacctattgaatgattcgatatataacatttccgaaggatataacataaataacctcaaggtaaatttccgcggccagaacgccaatctgaatccccgcattatcacacccataactggcaggggactcgtcaaaaagatcattaggttctgtaagaatattgtgtcggtgaaaggcattcgaaaaagtatctgcattgagattaacaatggagaactcttctttgttgcctcagaaaactcctacaatgacgataacatcaatacacccaaagaaatcgatgacaccgttacttccaacaataactatgagaacgacctggaccaggtgatccttaatttcaactctgagagcgccccaggattgtccgatgagaagcttaatctgaccattcagaacgacgcctacattcctaagtatgattccaatggcacaagtgatatcgaacagcatgacgtgaacgagttaaacgtgttcttttacttagatgcacaaaaagtccctgaaggagagaacaatgtgaacttaacctctagcattgacaccgccctcttggaacagcccaaaatatacactttcttttcttcagagtttataaacaatgtaaacaagcccgtgcaggctgcgctgttcgtgtcttggatccagcaagtgctggtggatttcacaaccgaagccaaccagaagtcaaccgtggacaagatagccgacatctccattgtcgtgccttatatcggcctcgctctgaacatcggaaacgaggcgcagaagggtaacttcaaggacgccctcgagctcctgggcgcgggtatcctgctcgagttcgagccggaattgctaatccctaccatccttgtgttcaccatcaaaagtttcctggggtcctctgacaacaagaacaaagttataaaggctatcaacaatgctttgaaagaacgcgatgagaagtggaaggaagtgtacagctttatcgtgtccaactggatgacaaagattaatacccagtttaacaagcggaaagagcagatgtatcaagcactgcagaatcaggtcaacgctattaagaccataattgagagcaagtacaatagttatactctggaggaaaagaacgaactgaccaacaaatacgatatcaagcagatcgaaaacgagttaaaccagaaggtgagcattgcaatgaataacatcgataggtttctcacagagtcttcaatctcttacttgatgaagttgattaatgaggtgaagattaacaaactgagagagtacgatgagaacgttaaaacttacctactgaattacataattcagcacgggtctatcctgggcgaatcccaacaggagcttaacagtatggtgactgatactctgaataactcgataccatttaagctgagttcatatactgatgacaagatcttgatctcatactttaacaagttctttaaacggatcaagtcgagctcagtgctgaacatgaggtataagaacgacaagtacgtcgataccagcggctatgatagcaacatcaacatcaacggggacgtgtacaagtaccccactaataaaaaccagttcggaatctataacgataagctaagcgaggtaaacattagccagaatgactacatcatttacgacaacaagtacaaaaacttcagtatatcgttctgggttcggataccgaattacgacaataagattgttaacgtaaataacgagtatacaatcattaactgtatgcgggataataactcagggtggaaagtatcactgaaccacaacgagatcatatggaccttgcaggacaacgcaggaatcaatcaaaagcttgcctttaattacgggaatgcgaatgggatttctgattacatcaataaatggatctttgtgactattacaaacgataggctcggtgactccaaactgtatataaatggaaatctgatagaccagaagagcatcctcaatctgggtaacatccatgtctcagataatatcctcttcaagattgttaattgttcttatacccggtatatcgggatccggtattttaatattttcgacaaggaactggacgaaacagaaatccagaccctctattctaacgaacctaacaccaatattttgaaggatttttggggtaattatctactctacgacaaggagtattacctgctcaatgtgcttaaaccaaacaatttcattgaccgtagaaaggactccacactctccattaacaatatcagaagtactatcttactggctaacagactgtatagcgggatcaaggtcaaaatccaaagggtcaacaatagcagtacaaatgacaaccttgtgcgaaagaacgatcaagtctacatcaacttcgtcgccagcaagacccatcttttccctctgtacgccgacactgctaccacgaataaggagaagacgatcaaaataagttctagtggcaaccgctttaaccaggtcgttgtgatgaattctgtcgggaataactgcacaatgaatttcaaaaacaataacggaaacaatatcggcttactcggattcaaagcagacaccgtggttgcttcaacgtggtattacacccatatgcgtgaccacaccaactccaatggctgcttctggaacttcatcagcgaagagcacggttggcaggaaaaatga

SEQ ID NO: 75 - DNA, 3759 bp, Artificial Sequence
  mat_peptide (1)...(3756): BoNT/E, M. musculus-modified 2
atgcccaaaatcaattcttttaactacaacgaccctgtgaacgacagaactattctttatatcaagccaggagggtgtcaggagttctataaatcattcaatataatgaagaacatctggatcattcccgagagaaacgtgataggcaccacacctcaagactttcacccccctacatctctgaagaacggcgacagttcctattacgacccaaactatctgcaatctgacgaagagaaggaccgattcttaaagatcgtgactaagatcttcaatcgtatcaacaataacttgtcaggagggatcctgttggaggaactgagcaaggctaatccttacctcggtaatgacaatacccccgacaaccagtttcatatcggcgacgcctcagctgtggagattaagttttcaaatggctctcaggatatcctcctgcctaatgtgatcattatgggcgccgagcctgatctatttgagacaaattcctctaacattagcctcaggaacaattatatgccttccaaccatggatttgggagcatcgccatagttactttcagcccagagtatagttttcgttttaacgataactctatgaacgaatttatccaggaccccgcactgactctcatgcacgagctcattcacagcctgcacgggctctacggcgcaaaagggattacaaccaagtacaccattacccagaagcaaaatcccctgattaccaacatccgaggaacaaacattgaggaatttcttactttcgggggtacagaccttaatatcattacttccgctcagagtaatgacatatacactaatctccttgccgactataagaaaatcgcatccaagctgagcaaggtgcaggtttctaaccccctcctgaatccgtacaaggacgtcttcgaggcgaaatatggcctggacaaggacgcctccggcatttacagtgtcaatatcaacaagtttaacgatatctttaagaaactctactcgtttacggagtttgacctggccaccaaattccaggtgaagtgcaggcaaacgtacatcgggcagtacaagtatttcaaactgagtaatctactcaacgactccatctacaacatttcagagggctataacatcaacaatttaaaggtgaatttcagaggccagaacgcaaacctgaaccccagaatcataacacccattaccggacgaggactagtgaaaaagatcattagattctgtaaaaatattgtttccgtaaaaggtatcaggaagtcgatctgcatagagatcaataacggtgaactgttctttgtggcttcagagaatagctacaacgatgacaacataaacacgccaaaagagattgacgataccgtgacatctaacaataactacgaaaacgacctggaccaggttatcttgaacttcaactctgagtctgctcctggtctgagcgatgaaaaacttaaccttacaattcagaatgacgcctatatacctaagtacgattctaatggtacttctgacatcgagcaacacgacgtaaacgagcttaacgttttcttttacctggatgcacagaaggtcccggaaggagagaataacgtgaatttgacgtcaagcatagataccgcgcttttagagcagccaaagatctatactttctttagttcagaatttatcaacaatgtgaacaagcccgtgcaggccgcacttttcgtgtcttggattcagcaagtccttgtcgattttacgaccgaggccaaccagaagagcacagttgataaaattgcagacatttcaatagtagtcccatacattggtcttgctctgaacatagggaatgaagcgcaaaagggcaatttcaaggacgctttggagctcctgggggctggcattctcctagagtttgagcccgaattattgatcccaactattctcgtgttcaccatcaaatccttcttgggatccagcgataataaaaacaaggttatcaaagcaatcaacaatgctctgaaggaaagagatgaaaagtggaaagaggtctactccttcatcgtatcaaactggatgactaagatcaacacccagtttaataagcgtaaggaacaaatgtaccaggccttacagaatcaggtgaacgccattaaaacaataatcgagtcgaaatataatagttatacactagaggaaaaaaatgaactgacaaacaaatacgatatcaaacagatcgaaaatgagctcaatcaaaaggttagtattgccatgaacaatatcgataggttcctgacggaatcaagcatctcctatttgatgaagttgattaacgaagtaaaaatcaataagctgcgcgagtatgacgagaacgtgaaaacatacctcctgaattatatcatacagcacggaagtatcctgggcgagagtcagcaagaactgaattcaatggttaccgataccctaaacaattcaatccctttcaagctgagttcctataccgacgataagatattgatatcttacttcaacaagtttttcaagcggataaaatctagctctgtcctaaacatgcggtacaaaaacgataagtatgtggacacctcgggctatgatagcaatataaatattaacggggacgtgtataaatacccaaccaacaagaaccagtttggcatttacaacgataaactgagtgaggttaatatctctcagaatgattacattatctacgataacaagtacaaaaatttcagcatctccttctgggtgaggatccctaattacgataacaagatcgtgaacgtcaataacgagtataccatcattaattgtatgcgagacaataactctggctggaaagtcagccttaatcataacgagataatctggactctgcaggataacgctggaatcaaccagaagctggcctttaattacgggaacgccaacggtattagcgattacatcaacaaatggatcttcgtgactattacgaatgataggctcggtgattccaagctctacattaatggcaacctgattgatcagaaaagcatcctgaatcttggaaacattcacgtttccgataatatactctttaaaattgtaaattgcagctatacacggtatattggaatcaggtacttcaacatcttcgataaggagctcgacgaaacagaaatccagaccttatatagtaacgagccgaatacaaacattttaaaggacttctggggtaactacctcctgtacgacaaggaatactatctgttgaacgtactgaagccaaataacttcattgatcgccggaaggacagtactctgtccattaacaatatcagatccactatcctgctagctaaccgcttgtactctgggataaaagtgaagatccagcgggtgaataactcaagcacaaatgacaacctggtgcggaagaatgaccaggtctatattaatttcgtcgcttccaagacccatctcttcccactgtatgcggacaccgccacaactaacaaggaaaaaacaatcaaaatcagtagctccggcaaccgcttcaaccaggtggtcgtgatgaacagcgtcggaaataactgtactatgaattttaagaacaataacggaaacaatattgggctgttgggcttcaaggccgacaccgtggtcgcatccacgtggtactacacccatatgcgcgatcataccaactcgaatgggtgcttttggaacttcatcagcgaagagcacggatggcaagaaaagtga

SEQ ID NO: 76 - DNA, 3759 bp, Artificial Sequence
  mat_peptide (1)...(3756): BoNT/E, M. musculus-modified 3
atgcctaagattaattccttcaactataacgaccctgtgaatgaccggactattctttatatcaaacctggcggatgtcaggagttctacaaaagctttaatattatgaaaaatatctggatcattccagaacgtaacgtgatcgggaccacacctcaggatttccatccccctactagcctgaagaacggggacagttcttattacgatcctaattatctgcagtccgacgaagagaaggacaggttcctcaaaattgtgacaaaaatcttcaataggatcaacaataacctgagcggtggcatcctgctcgaggaactgagcaaggcaaatccctatctgggcaacgacaatacccccgataaccagtttcacatcggcgacgcctcagccgtggagatcaagttttccaacggaagccaggatatcctgttgcctaacgtgattatcatgggcgccgaacctgatctgttcgagaccaattccagcaatatctcactcagaaataactacatgccctctaaccacggatttggctccatcgcaattgtgactttcagccccgaatacagctttcggtttaacgataactcaatgaacgagtttatccaggatccagctctgacccttatgcatgaactcatccatagcctgcacggactgtacggcgccaaggggatcaccactaagtacaccatcacccagaaacagaacccactgattactaatatcagggggaccaatatcgaagagttcctcaccttcgggggcaccgacctcaacatcattacttctgctcagagcaacgacatttataccaatctcctggccgactataagaaaatcgctagtaagctcagtaaggtgcaggtgtcaaacccccttctgaatccatacaaagatgtgtttgaggcaaagtacggcctggacaaggacgcctccggaatctactctgtcaacatcaataagttcaacgacattttcaagaaactgtactccttcactgagttcgatttggccacaaagttccaggtgaagtgcagacagacttatatcggacagtataaatactttaaactcagtaatcttctgaacgattccatctataacatctccgaaggctacaacattaacaatctgaaggtgaatttccgcgggcagaatgccaacctgaatccacgcatcattacacctatcacagggaggggacttgtgaagaaaatcattcgattttgtaagaatatcgtcagcgtgaagggaattcggaagagcatttgcattgagatcaacaatggtgaactgttctttgtggctagcgagaattcttacaacgacgataacattaacacaccaaaagagattgacgatacagttacaagcaacaataactatgagaacgatctggaccaggtcatccttaactttaattcagagtctgctcccggtctgagcgacgaaaaactgaatctgacaatccagaacgatgcctatattcccaagtacgattcaaacggcacttctgacatcgagcagcatgatgtgaatgaactcaatgtgttcttttacctggacgcccagaaggtcccagagggcgagaacaatgtgaacctgaccagcagtatcgatacagctctgctcgagcagcccaaaatttatacattcttttcttccgaatttattaacaatgtgaacaaaccagtgcaggccgctctttttgtttcttggatccagcaggtgctggttgactttaccactgaggccaaccagaaaagtaccgtcgacaaaattgctgacatttcaattgttgtcccatacatcggactcgctctgaacatcggaaacgaggcacagaaaggaaacttcaaggacgccttggaacttttgggggctggcattctgttggagttcgaacctgagcttctgattcctactatcctcgtgttcaccattaaatcctttttgggttccagtgacaataaaaacaaggtcatcaaggcaatcaataacgccctgaaggaacgcgatgaaaagtggaaggaagtctatagctttattgtgtccaattggatgactaaaatcaacactcagttcaacaagagaaaggagcagatgtatcaggccctccagaaccaggttaacgccattaagaccatcattgaatcaaagtataattcctataccttggaggaaaaaaacgagttgactaataagtacgacatcaagcagatcgagaacgaactgaaccagaaggtgtcaatcgccatgaataacattgacagattcttgactgagagctctatttcatatctgatgaagctgatcaacgaagtgaagatcaacaagctgcgcgagtatgacgagaatgttaaaacatatttgctgaactacatcattcagcacgggagcatccttggggagtctcagcaggagctgaattctatggtgaccgataccctgaacaatagcattccattcaagctgtcctcttacacagacgataaaatcctgatctcctacttcaacaaattctttaaacggattaagagttctagtgttctgaatatgcggtacaagaatgacaagtacgtcgataccagcggatacgattctaacatcaatatcaatggagacgtctacaaatatcctaccaataagaatcagttcggcatctacaatgataagctgagcgaagtcaacatcagccagaacgactacatcatttacgataataagtacaagaactttagcatcagcttctgggttaggatccctaactacgacaacaaaatcgtgaatgttaacaatgaatacaccatcattaactgtatgagggacaacaattccggttggaaggtgtccctgaaccataatgagatcatttggacactgcaggacaacgcaggtatcaatcagaagctggcttttaactatggcaacgcaaacggcatctcagactatattaacaaatggatcttcgtgaccatcacaaacgatcgactgggggatagcaaactgtacatcaacgggaacttgatcgaccagaagagcatcctcaacctcggtaacatccacgtgagtgacaacatcctgttcaagattgtgaattgttcttacacccggtacatcggaatccgttatttcaacatctttgataaggagctggacgagaccgaaatccagacactctactctaatgagcccaataccaacatcctgaaggatttctgggggaattacctgctttacgataaggaatattacctgctcaatgtcttgaagcctaataactttatcgatcgcagaaaagattcaaccttgagtatcaacaatattagaagtaccatccttctggccaacagactctattccggcatcaaggttaaaatccagagggtcaataacagttccaccaatgataacctcgtccgaaaaaacgaccaggtgtatattaattttgtggctagtaaaacccacctgttccccctttatgcagatactgcaaccacaaacaaggagaaaacaatcaagatctccagttcaggaaatcgattcaatcaggttgtggtcatgaattcagtgggcaacaattgcaccatgaacttcaagaacaataacggcaacaatatcggtctcctggggtttaaagccgacacagtggtggcctctacctggtactacactcatatgcgtgaccacacaaatagcaatggttgcttctggaactttatctctgaagagcacggttggcaggagaagtga

SEQ ID NO: 77 - DNA, 3759 bp, Artificial Sequence
  mat_peptide (1)...(3756): BoNT/E, R. norvegicus-modified 1
atgcccaaaattaactccttcaactacaacgatcctgttaacgacaggacaattctttatatcaaaccgggcggttgccaggaattctacaagtctttcaacatcatgaaaaatatctggatcattcccgagcggaacgtgattggtactacacctcaagattttcatcccccaacctccctcaagaacggcgatagctcctactatgatccaaactatttgcagagcgacgaggaaaaggatagatttctcaagattgtcactaagattttcaacaggatcaacaataacctgtcaggtgggatcctccttgaggaactcagcaaagccaacccatacttgggaaacgacaacacccctgataaccagtttcacattggcgacgcctctgcagtagagattaagttctccaacggcagccaggacatcctactgcccaatgtcatcattatgggagccgaacccgacctgttcgaaacaaatagttctaatatctcactgcgtaacaattatatgccatccaatcacgggtttggcagcatcgcaatcgtgacctttagtcccgaatacagctttcgctttaacgacaacagtatgaatgaattcattcaggaccctgctttgacactaatgcatgaactgattcatagtctgcacggcctgtatggagcgaaaggtatcactacgaaatacacaattactcagaaacagaatcctttaattaccaacatcaggggcaccaacatcgaggaattcctgacatttggcggaacagatctgaacattatcacatcggctcagtccaatgatatctacactaacctcctggccgactacaagaaaattgccagcaaattgagcaaagtgcaagtgtctaatccgttgctgaacccgtacaaggatgtgttcgaggctaagtatgggttagataaggacgcgtcaggaatctattcagtcaacattaataaatttaacgatatcttcaagaaactctactcttttaccgagtttgatctggccacgaagtttcaagtgaaatgccggcaaacctatatcggacaatacaaatactttaaactatcgaacttgctgaacgatagcatctacaatatatctgaaggctacaatatcaacaatctgaaggtcaacttccgtggccagaacgctaatcttaacccaaggattataaccccaatcacagggcgagggctggttaagaaaattatccggttctgcaagaatatagtgtcagtgaagggaattcgcaagtctatctgcatcgaaatcaataacggtgaactgtttttcgtcgctagcgaaaactcatacaacgatgacaacataaacactccgaaggaaattgacgataccgtgaccagcaacaataactatgagaacgacctcgatcaagtgatcctgaacttcaattcagagtccgcccctggactgtctgacgaaaagttgaacctgacaattcagaatgacgcgtatatccctaaatatgactcgaacggcactagcgacatcgagcagcatgacgtgaacgagctcaacgtctttttctatcttgacgctcagaaggtacctgagggggagaataacgtcaatcttacttcctcaattgacacagccctgctcgagcagccaaagatttatacctttttcagcagtgagttcataaacaatgtaaataagcccgtgcaggcagctctgtttgtttcttggatccagcaagtactcgtggatttcaccacggaggccaaccagaagtctacggttgataaaatcgcggacatatccattgtggtcccctacatcggtctggccttgaacatcggcaatgaggcacagaagggaaacttcaaggacgcacttgagttgctgggggcaggtattctgttggaattcgagcccgagctcctgatcccaactattctcgtgttcactatcaagtctttcctcgggtcaagtgataataagaacaaggtgatcaaagccatcaataacgcactcaaggagagagacgaaaagtggaaggaggtgtactcctttatagttagtaactggatgaccaagatcaatacacaatttaataaaagaaaagagcagatgtatcaggcactgcagaatcaggttaacgccatcaagaccatcattgagagcaagtacaattcttacaccctcgaggaaaagaacgaactaactaataagtatgacattaagcagattgagaacgagctcaaccagaaggtgtccatcgctatgaataacatcgacagatttttaactgagagcagtattagctacctcatgaagctgattaatgaagttaagatcaacaagcttcgggagtatgatgagaatgttaagacctacctgcttaactacatcattcaacacggcagtattctaggagaaagccagcaagaacttaattcgatggtcacagatacactcaacaatagcattcctttcaaactatctagctacacggatgacaagatcctgatctcttactttaataaattctttaagaggatcaagagttctagcgtgctgaacatgcgctacaaaaacgacaagtacgtcgacacctccgggtatgactcaaatataaacatcaatggcgacgtatacaagtatccaaccaataaaaatcagttcggtatctacaatgacaagctgagtgaggtgaacatttctcagaacgactacattatctatgacaacaaatataaaaatttctctatctcattttgggtgcgaatccccaactacgacaataagatcgtcaacgtcaacaatgagtatacgataatcaactgtatgcgggataacaatagcggctggaaagtctccctgaatcacaacgagatcatttggaccctgcaggacaacgctggtatcaaccagaaactggccttcaactacggcaacgctaacggtatctccgactacattaacaaatggatcttcgttaccataaccaatgacagacttggggactccaagctatacatcaatggaaatttgatcgaccaaaagtccatcctgaacctggggaacattcacgtctccgacaacatactgtttaagattgtgaattgtagttacacacgatacataggaatcagatacttcaatatatttgataaggaattagacgaaaccgaaattcagactctttactctaacgagcccaataccaatatcctgaaagatttctggggcaactaccttctgtatgacaaagagtattacctgctcaacgtgttaaagcctaacaatttcatcgatcgccgtaaggattccaccctcagcataaataacatccgctccacaatcttgctcgccaaccgactctattccgggatcaaagtgaagatacagcgcgtgaacaattccagcactaacgataacctggtccgcaagaacgatcaggtctacatcaatttcgtggcctccaaaacccatctgttccctctgtatgccgataccgctaccacgaacaaggagaagacaatcaaaatctcttcgagcggaaaccggttcaaccaggtggttgtgatgaattccgtgggcaataactgtactatgaatttcaaaaacaataacgggaataacatcggcctgttgggctttaaggctgacacggtcgtagcctccacttggtattacactcacatgagggatcacaccaacagtaacggatgcttctggaacttcatctcagaggagcacggttggcaggagaagtga

SEQ ID NO: 78 - DNA, 3759 bp, Artificial Sequence
  mat_peptide (1)...(3756): BoNT/E, R. norvegicus-modified 2
atgcctaagatcaacagcttcaactacaacgaccctgtgaacgaccgcaccatcctgtacatcaagcctggcggctgccaggagttctacaagagcttcaacatcatgaagaacatctggatcatccctgagcgcaacgtgatcggcaccacccctcaggacttccaccctcctaccagcctgaagaacggcgacagcagctactacgaccctaactacctgcagagcgacgaggagaaggaccgcttcctgaagatcgtgaccaagatcttcaaccgcatcaacaacaacctgagcggcggcatcctgctggaggagctgagcaaggccaacccttacctgggcaacgacaacacccctgacaaccagttccacatcggcgacgccagcgccgtggagatcaagttcagcaacggcagccaggacatcctgctgcctaacgtgatcatcatgggcgccgagcctgacctgttcgagaccaacagcagcaacatcagcctgcgcaacaactacatgcctagcaaccacggcttcggcagcatcgccatcgtgaccttcagccctgagtacagcttccgcttcaacgacaacagcatgaacgagttcatccaggaccctgccctgaccctgatgcacgagctgatccacagcctgcacggcctgtacggcgccaagggcatcaccaccaagtacaccatcacccagaagcagaaccctctgatcaccaacatccgcggcaccaacatcgaggagttcctgaccttcggcggcaccgacctgaacatcatcaccagcgcccagagcaacgacatctacaccaacctgctggccgactacaagaagatcgccagcaagctgagcaaggtgcaggtgagcaaccctctgctgaacccttacaaggacgtgttcgaggccaagtacggcctggacaaggacgccagcggcatctacagcgtgaacatcaacaagttcaacgacatcttcaagaagctgtacagcttcaccgagttcgacctggccaccaagttccaggtgaagtgccgccagacctacatcggccagtacaagtacttcaagctgagcaacctgctgaacgacagcatctacaacatcagcgagggctacaacatcaacaacctgaaggtgaacttccgcggccagaacgccaacctgaaccctcgcatcatcacccctatcaccggccgcggcctggtgaagaagatcatccgcttctgcaagaacatcgtgagcgtgaagggcatccgcaagagcatctgcatcgagatcaacaacggcgagctgttcttcgtggccagcgagaacagctacaacgacgacaacatcaacacccctaaggagatcgacgacaccgtgaccagcaacaacaactacgagaacgacctggaccaggtgatcctgaacttcaacagcgagagcgcccctggcctgagcgacgagaagctgaacctgaccatccagaacgacgcctacatccctaagtacgacagcaacggcaccagcgacatcgagcagcacgacgtgaacgagctgaacgtgttcttctacctggacgcccagaaggtgcctgagggcgagaacaacgtgaacctgaccagcagcatcgacaccgccctgctggagcagcctaagatctacaccttcttcagcagcgagttcatcaacaacgtgaacaagcctgtgcaggccgccctgttcgtgagctggatccagcaggtgctggtggacttcaccaccgaggccaaccagaagagcaccgtggacaagatcgccgacatcagcatcgtggtgccttacatcggcctggccctgaacatcggcaacgaggcccagaagggcaacttcaaggacgccctggagctgctgggcgccggcatcctgctggagttcgagcctgagctgctgatccctaccatcctggtgttcaccatcaagagcttcctgggcagcagcgacaacaagaacaaggtgatcaaggccatcaacaacgccctgaaggagcgcgacgagaagtggaaggaggtgtacagcttcatcgtgagcaactggatgaccaagatcaacacccagttcaacaagcgcaaggagcagatgtaccaggccctgcagaaccaggtgaacgccatcaagaccatcatcgagagcaagtacaacagctacaccctggaggagaagaacgagctgaccaacaagtacgacatcaagcagatcgagaacgagctgaaccagaaggtgagcatcgccatgaacaacatcgaccgcttcctgaccgagagcagcatcagctacctgatgaagctgatcaacgaggtgaagatcaacaagctgcgcgagtacgacgagaacgtgaagacctacctgctgaactacatcatccagcacggcagcatcctgggcgagagccagcaggagctgaacagcatggtgaccgacaccctgaacaacagcatccctttcaagctgagcagctacaccgacgacaagatcctgatcagctacttcaacaagttcttcaagcgcatcaagagcagcagcgtgctgaacatgcgctacaagaacgacaagtacgtggacaccagcggctacgacagcaacatcaacatcaacggcgacgtgtacaagtaccctaccaacaagaaccagttcggcatctacaacgacaagctgagcgaggtgaacatcagccagaacgactacatcatctacgacaacaagtacaagaacttcagcatcagcttctgggtgcgcatccctaactacgacaacaagatcgtgaacgtgaacaacgagtacaccatcatcaactgcatgcgcgacaacaacagcggctggaaggtgagcctgaaccacaacgagatcatctggaccctgcaggacaacgccggcatcaaccagaagctggccttcaactacggcaacgccaacggcatcagcgactacatcaacaagtggatcttcgtgaccatcaccaacgaccgcctgggcgacagcaagctgtacatcaacggcaacctgatcgaccagaagagcatcctgaacctgggcaacatccacgtgagcgacaacatcctgttcaagatcgtgaactgcagctacacccgctacatcggcatccgctacttcaacatcttcgacaaggagctggacgagaccgagatccagaccctgtacagcaacgagcctaacaccaacatcctgaaggacttctggggcaactacctgctgtacgacaaggagtactacctgctgaacgtgctgaagcctaacaacttcatcgaccgccgcaaggacagcaccctgagcatcaacaacatccgcagcaccatcctgctggccaaccgcctgtacagcggcatcaaggtgaagatccagcgcgtgaacaacagcagcaccaacgacaacctggtgcgcaagaacgaccaggtgtacatcaacttcgtggccagcaagacccacctgttccctctgtacgccgacaccgccaccaccaacaaggagaagaccatcaagatcagcagcagcggcaaccgcttcaaccaggtggtggtgatgaacagcgtgggcaacaactgcaccatgaacttcaagaacaacaacggcaacaacatcggcctgctgggcttcaaggccgacaccgtggtggccagcacctggtactacacccacatgcgcgaccacaccaacagcaacggctgcttctggaacttcatcagcgaggagcacggctggcaggagaagtga

SEQ ID NO: 79 - DNA, 3759 bp, Artificial Sequence
  mat_peptide (1)...(3756): BoNT/E, R. norvegicus-modified 3
atgcccaaaatcaattctttcaactataacgacccagtaaacgatcgcaccatcttgtatatcaagcctgggggctgtcaggaattttataagtctttcaatattatgaaaaacatatggatcattccggagcgaaacgtgatcggaacgacaccacaggactttcatcccccaaccagcctgaaaaatggagatagttcttattacgacccgaactacctccagtcggatgaagagaaggacagattcttgaagatcgtcacaaagattttcaaccgtatcaataacaatttgagcggtggaatcttattggaagagctgagtaaggctaacccctacctggggaacgataatacccctgataaccaattccacatcggagacgccagtgccgtagagatcaaattcagcaacgggtcccaagacattttgctgcctaacgttatcattatgggggccgagcctgatcttttcgagacaaatagctctaacatcagcctgcgaaacaattacatgccctccaatcacggattcggaagcattgctattgtgacattttcacctgaatacagcttccgctttaatgacaacagtatgaacgagtttattcaggatcctgctctgactttaatgcatgagttgatccactcactacacggactttatggcgctaaggggatcactaccaagtatactatcacccagaagcagaacccgctcattaccaatatccggggcactaatatcgaggaattcctgactttcggaggtacggaccttaacattataacctcggcacagtctaacgacatctacacaaacctactggcagattacaagaaaatcgctagtaaactcagcaaggtgcaagtctccaacccccttctcaacccctacaaagacgtgtttgaggccaagtatggtctggataaggatgcgagtggcatctattcagtgaacatcaacaagttcaatgacatttttaaaaagctttatagcttcactgaattcgatctggctaccaaatttcaggttaagtgtaggcaaacttacattggccagtataagtactttaaactgtctaacctgttaaacgacagcatctacaacataagtgagggctacaacatcaataacctgaaggtgaattttcgcggtcaaaatgctaatttgaatcccagaataattacaccaattactggcagggggcttgtgaagaaaattatccgcttctgcaagaacattgtgtctgtcaagggcatccggaagtcgatctgtatcgaaatcaataacggagagctcttctttgttgcatccgagaacagctacaacgacgataacataaacaccccaaaagagatagacgataccgtgacctcaaacaataactacgagaacgatctggatcaagtgattctaaattttaattctgagtctgcacccggcttgtccgacgagaagcttaatctcaccattcagaacgacgcctatatcccaaaatacgatagcaatggaacaagtgatatcgagcagcatgacgtgaatgaactaaacgtcttcttttacctggacgcgcagaaggtgccagagggtgaaaacaatgtgaacctcacttcctcaattgacacggccctgcttgaacagcccaaaatctataccttcttttcttccgagttcattaacaatgtcaataagcctgttcaagctgccctgtttgtctcatggattcagcaagtactcgtcgactttacgaccgaggcaaaccagaagtctacggttgacaagatcgccgacattagcatcgtggttccttatataggtctggcattgaatattgggaatgaggcccagaagggcaacttcaaggacgccctggagcttctcggcgcgggcatcctgctcgaatttgaaccagagctgctcatacctaccattcttgtcttcactattaagagcttcctgggctcaagtgacaacaagaacaaggttatcaaggctattaacaatgcactgaaggaaagagatgaaaagtggaaggaagtctattcctttatcgtgtcgaactggatgaccaagattaacacacagttcaataaaagaaaggagcagatgtaccaggccctgcagaatcaggtcaacgccataaaaactatcatagagtctaaatacaattcatatacccttgaggaaaaaaatgaactcacaaacaaatacgatatcaaacaaatagaaaatgaactgaatcagaaagtgagcatcgccatgaacaatatcgatcggtttctcaccgagtccagcatctcctatctcatgaaactgatcaacgaggtgaagattaacaaactgcgggaatacgacgagaatgtcaagacatacctgttgaactacatcattcagcacggaagcatcctaggtgagtctcagcaagagctgaacagcatggtgactgacacactgaacaattctatcccgttcaaattgagttcttacaccgacgataagattctgatctcttacttcaacaagtttttcaagcggatcaagtcatcgagcgtcctgaacatgaggtacaaaaacgacaaatacgtcgataccagcgggtacgattcaaacatcaacatcaacggggatgtctacaaatatcctactaataagaaccagtttggaatttacaacgataagctttccgaagtgaatatctcccaaaacgactacatcatttacgacaacaagtacaaaaatttttccatctccttctgggtgaggatccctaactacgataacaagattgtcaatgtaaacaatgaatataccatcattaactgcatgcgggacaacaatagtggctggaaggtgtccctgaaccataacgagattatctggaccttgcaggacaacgccggtatcaaccagaagctggctttcaactatggtaatgcaaatggcatctcagactacatcaacaaatggatctttgtgaccattacaaatgaccgcctgggcgactccaaattatatatcaatgggaacctcatcgaccagaagtccatcctgaacctaggaaatatccatgtttccgacaacatcctcttcaagatagtgaactgctcttacactcgctatatcggaatccgctattttaacatcttcgacaaagagctggatgagaccgagatccagacactgtacagcaatgagccaaacacaaacatcctgaaggatttttggggtaattacctcctgtatgataaagaatactatctgttgaatgtactgaagcccaataacttcattgaccgaaggaaggactccacgctgagcattaacaatattagaagtacgattctcctagccaaccgtttatattccggcataaaggtcaagatccagcgtgttaacaattcctctaccaacgataacctcgtaaggaagaatgaccaggtgtacataaacttcgttgcttccaaaactcacctcttccccctgtatgctgatactgcgaccacgaacaaagagaagactatcaagataagtagctccggcaacagattcaaccaggtggtcgtgatgaattctgtgggtaataactgcacaatgaattttaaaaataacaatgggaacaatatcgggctcctcgggttcaaggccgacaccgtggtggccagcacatggtactacacacacatgcgagaccacaccaattccaacggctgcttctggaacttcatttcagaggaacacggctggcaggaaaaatga

SEQ ID NO: 80 - DNA, 3759 bp, Artificial Sequence
  CDS (1)...(3759): BoNT/E, C. griseus-modified 1
atgccaaaaattaacagttttaattacaatgatcccgtgaacgaccgcacaatcctttacattaaacccggtggatgtcaggagttctacaaaagctttaacatcatgaagaacatctggataatcccagaacgtaacgtgattggaacaacccctcaggacttccatccaccgacaagtttaaaaaatggcgacagctcttattacgaccccaactaccttcagagtgatgaagagaaggaccgattcctgaagatcgtcacaaaaatctttaacaggatcaacaataacctgagtggcggaatcctgttggaggaactaagtaaagcaaatccttacctcgggaacgacaacacacccgacaaccagttccacatcggcgatgccagcgctgtcgaaatcaaatttagcaacgggagccaggacatcctgttgcctaacgtcatcattatgggtgctgagccagaccttttcgaaaccaactccagcaacatcagcctcaggaacaattacatgccgagcaatcacggctttggctctattgccatcgtgacgttttcgcccgagtacagcttcagatttaatgacaacagcatgaacgaattcattcaagatccagctctcacactcatgcatgaactcattcacagcctgcacgggctctacggcgctaagggcataactaccaagtatactatcactcagaagcaaaacccactgatcacaaatatccggggcaccaacatcgaggaattcctcacattcggagggactgacttaaatatcattacgagtgctcaatccaacgatatctacactaatcttctggccgattataagaaaattgcatcgaagctcagtaaggtgcaagtgtcaaatcctctcctgaatccatataaagacgtgttcgaggcgaaatatggcctggataaggatgccagtggtatctactcggtgaatatcaacaagtttaatgatatctttaagaaactatactctttcaccgagtttgatcttgcaactaagtttcaggtcaagtgtcggcagacttacatcgggcagtataagtactttaagctgtcaaatctgttgaacgactccatctataatatctcagaaggctacaacataaacaatctgaaagtaaacttccgcggccagaacgccaacctaaacccccggatcattacgccgataaccggcagagggttagtgaaaaagatcattcgattctgcaaaaatatcgtgtctgttaaaggaatcaggaagtccatctgtatcgagattaacaatggcgaattattctttgtagccagcgagaattcttacaacgatgacaacatcaatacacctaaggagattgacgatacagttaccagcaacaataactatgagaatgatttggaccaggtgattctaaattttaacagtgaatccgcccccggtctatctgatgagaagttgaatctgaccatccagaatgatgcttatatcccaaagtacgattcaaacgggacttcggatattgagcagcatgacgtcaatgaactgaatgtgttcttttatctggacgctcagaaagtccccgagggtgagaacaatgttaatctgacatcctctatagacaccgcattgctcgaacagcctaaaatctacacctttttctccagtgaatttatcaacaatgtgaacaaacctgtacaggccgcactgttcgtttcttggattcagcaagttttggttgactttaccactgaggccaatcaaaagagtactgtggacaaaatcgccgacatctcgattgtggtcccatacataggattggcactgaacatcggaaacgaggctcagaagggtaatttcaaggacgccctcgagctgttgggagcaggcatactgcttgagttcgaacccgagctgttaattcctaccattctggttttcactatcaaatcctttctcggatcttccgacaataagaataaggtcatcaaggctataaacaatgcccttaaggagcgcgatgagaagtggaaagaggtatactcttttattgtgagtaactggatgacaaaaattaatacccagtttaataagagaaaggaacagatgtaccaagctctccagaatcaggtcaacgctataaaaaccataattgagtccaaatacaatagttatactctggaagagaagaacgagctaacaaataaatatgatatcaagcagattgagaatgaactcaatcagaaggtctcaattgccatgaacaatattgataggttcctaacagaatcatctatctcttacctcatgaagctgataaatgaggtcaagattaacaaattgcgggagtacgacgaaaatgttaaaacctaccttttgaattacataattcagcacggaagcatcctgggcgaatcacagcaagaactcaattccatggttacggatacactgaacaattccatcccattcaagttatcttcctatactgacgataagatattgatttcttacttcaataaattctttaagagaatcaagagctcctctgtgctaaatatgcgttacaagaacgataaatatgtggacacttcagggtacgattcaaacattaacatcaatggtgatgtgtataagtatcccaccaacaaaaaccaattcgggatatacaatgacaagctgagtgaggtgaacatctctcagaacgactatattatctacgacaataaatacaagaacttcagcatttctttctgggtgcgcattcctaattatgacaacaagatcgtgaatgtgaataacgagtacacaatcattaactgtatgcgcgacaataactccggctggaaggtaagtctcaaccacaacgagattatctggacccttcaggataatgcgggaattaaccagaaactggccttcaattatggcaatgcgaacggaatctctgattacatcaacaagtggatctttgtgacaataactaatgatcggctgggggacagcaagctctatatcaacgggaacctgattgatcagaagtccattttgaacctgggaaatattcatgtgtcagacaatatcctttttaagatagtcaactgcagctacacgcgttacatcggtattcgatatttcaacatttttgataaggaattggacgaaacggagatccagacactgtattcaaacgagcctaacactaacattctgaaagatttctgggggaactatcttctgtatgataaagagtattaccttctgaacgtcctgaaacctaataacttcatcgatcgcagaaaggattccaccttgtctatcaataacattaggtccaccatacttctggcaaatcgactttattctggaataaaggtcaagatccagagggtgaacaattcctcaaccaatgacaacctggtgcgtaaaaacgatcaggtgtatattaactttgtggcatccaagactcatctgttccccctctatgctgacaccgctacaaccaataaggaaaaaaccattaagattagctcatctggtaatagatttaatcaagtagtcgtaatgaatagcgttggcaacaattgcaccatgaacttcaaaaacaacaacggtaacaacatcggcctgctgggatttaaggcagacaccgttgtggcctccacctggtattacacacacatgcgggaccacactaacagcaacggttgcttttggaacttcatctccgaagagcatggttggcaggagaagtga

SEQ ID NO: 81 - DNA, 3759 bp, Artificial Sequence
  mat_peptide (1)...(3756): BoNT/E, C. griseus-modified 2
atgcctaagatcaactccttcaactacaacgaccctgtgaacgacagaaccatcctgtacatcaagcctggcggctgtcaggagttctacaagtccttcaacatcatgaagaacatctggatcatccctgagagaaacgtgatcggcaccacccctcaggacttccaccctcctacctccctgaagaacggcgactcctcctactacgaccctaactacctgcagtccgacgaggagaaggacagattcctgaagatcgtgaccaagatcttcaacagaatcaacaacaacctgtccggcggcatcctgctggaggagctgtccaaggccaacccttacctgggcaacgacaacacccctgacaaccagttccacatcggcgacgcctccgccgtggagatcaagttctccaacggctcccaggacatcctgctgcctaacgtgatcatcatgggcgccgagcctgacctgttcgagaccaactcctccaacatctccctgagaaacaactacatgccttccaaccacggcttcggctccatcgccatcgtgaccttctcccctgagtactccttcagattcaacgacaactccatgaacgagttcatccaggaccctgccctgaccctgatgcacgagctgatccactccctgcacggcctgtacggcgccaagggcatcaccaccaagtacaccatcacccagaagcagaaccctctgatcaccaacatcagaggcaccaacatcgaggagttcctgaccttcggcggcaccgacctgaacatcatcacctccgcccagtccaacgacatctacaccaacctgctggccgactacaagaagatcgcctccaagctgtccaaggtgcaggtgtccaaccctctgctgaacccttacaaggacgtgttcgaggccaagtacggcctggacaaggacgcctccggcatctactccgtgaacatcaacaagttcaacgacatcttcaagaagctgtactccttcaccgagttcgacctggccaccaagttccaggtgaagtgtagacagacctacatcggccagtacaagtacttcaagctgtccaacctgctgaacgactccatctacaacatctccgagggctacaacatcaacaacctgaaggtgaacttcagaggccagaacgccaacctgaaccctagaatcatcacccctatcaccggcagaggcctggtgaagaagatcatcagattctgtaagaacatcgtgtccgtgaagggcatcagaaagtccatctgtatcgagatcaacaacggcgagctgttcttcgtggcctccgagaactcctacaacgacgacaacatcaacacccctaaggagatcgacgacaccgtgacctccaacaacaactacgagaacgacctggaccaggtgatcctgaacttcaactccgagtccgcccctggcctgtccgacgagaagctgaacctgaccatccagaacgacgcctacatccctaagtacgactccaacggcacctccgacatcgagcagcacgacgtgaacgagctgaacgtgttcttctacctggacgcccagaaggtgcctgagggcgagaacaacgtgaacctgacctcctccatcgacaccgccctgctggagcagcctaagatctacaccttcttctcctccgagttcatcaacaacgtgaacaagcctgtgcaggccgccctgttcgtgtcctggatccagcaggtgctggtggacttcaccaccgaggccaaccagaagtccaccgtggacaagatcgccgacatctccatcgtggtgccttacatcggcctggccctgaacatcggcaacgaggcccagaagggcaacttcaaggacgccctggagctgctgggcgccggcatcctgctggagttcgagcctgagctgctgatccctaccatcctggtgttcaccatcaagtccttcctgggctcctccgacaacaagaacaaggtgatcaaggccatcaacaacgccctgaaggagagagacgagaagtggaaggaggtgtactccttcatcgtgtccaactggatgaccaagatcaacacccagttcaacaagagaaaggagcagatgtaccaggccctgcagaaccaggtgaacgccatcaagaccatcatcgagtccaagtacaactcctacaccctggaggagaagaacgagctgaccaacaagtacgacatcaagcagatcgagaacgagctgaaccagaaggtgtccatcgccatgaacaacatcgacagattcctgaccgagtcctccatctcctacctgatgaagctgatcaacgaggtgaagatcaacaagctgagagagtacgacgagaacgtgaagacctacctgctgaactacatcatccagcacggctccatcctgggcgagtcccagcaggagctgaactccatggtgaccgacaccctgaacaactccatccctttcaagctgtcctcctacaccgacgacaagatcctgatctcctacttcaacaagttcttcaagagaatcaagtcctcctccgtgctgaacatgagatacaagaacgacaagtacgtggacacctccggctacgactccaacatcaacatcaacggcgacgtgtacaagtaccctaccaacaagaaccagttcggcatctacaacgacaagctgtccgaggtgaacatctcccagaacgactacatcatctacgacaacaagtacaagaacttctccatctccttctgggtgagaatccctaactacgacaacaagatcgtgaacgtgaacaacgagtacaccatcatcaactgtatgagagacaacaactccggctggaaggtgtccctgaaccacaacgagatcatctggaccctgcaggacaacgccggcatcaaccagaagctggccttcaactacggcaacgccaacggcatctccgactacatcaacaagtggatcttcgtgaccatcaccaacgacagactgggcgactccaagctgtacatcaacggcaacctgatcgaccagaagtccatcctgaacctgggcaacatccacgtgtccgacaacatcctgttcaagatcgtgaactgttcctacaccagatacatcggcatcagatacttcaacatcttcgacaaggagctggacgagaccgagatccagaccctgtactccaacgagcctaacaccaacatcctgaaggacttctggggcaactacctgctgtacgacaaggagtactacctgctgaacgtgctgaagcctaacaacttcatcgacagaagaaaggactccaccctgtccatcaacaacatcagatccaccatcctgctggccaacagactgtactccggcatcaaggtgaagatccagagagtgaacaactcctccaccaacgacaacctggtgagaaagaacgaccaggtgtacatcaacttcgtggcctccaagacccacctgttccctctgtacgccgacaccgccaccaccaacaaggagaagaccatcaagatctcctcctccggcaacagattcaaccaggtggtggtgatgaactccgtgggcaacaactgtaccatgaacttcaagaacaacaacggcaacaacatcggcctgctgggcttcaaggccgacaccgtggtggcctccacctggtactacacccacatgagagaccacaccaactccaacggctgtttctggaacttcatctccgaggagcacggctggcaggagaagtga

SEQ ID NO: 82 - DNA, 3759 bp, Artificial Sequence
  mat_peptide (1)...(3756): BoNT/E, C. griseus-modified 3
atgcctaaaattaactccttcaattacaatgaccctgtaaacgaccgcaccatcttatacatcaaacctggagggtgccaagaattctataaatctttcaatatcatgaaaaacatttggatcattccagagcggaatgtgatcggtaccacaccccaggacttccatcctcccacgtcattgaagaacggcgactcgagttattacgatcctaactacctgcagagcgacgaggaaaaggacaggttcctcaaaattgtgaccaagatctttaacagaatcaacaataacctttcaggaggcatccttttggaggaactctccaaggccaacccatacctgggcaacgataacacgcctgataaccaatttcacattggcgatgctagcgctgtggagatcaagttttctaatggatctcaggatattttgctgccgaatgtgatcattatgggggcagagccagatctgttcgaaactaattctagcaatatctcactccggaacaattacatgcctagcaatcatggtttcggaagcattgcaatcgtcacctttagtccggagtactcattcaggttcaatgataactctatgaatgaatttattcaggaccccgccctcactctcatgcatgaattaatccatagcctgcatgggctttatggcgctaagggaattacaaccaagtacacaattactcagaagcaaaatcccctgataaccaacatcagaggaactaatatcgaagagtttctgactttcgggggaacggacctcaacatcataacctcggcccaatccaacgatatctataccaatttgctggcagactacaagaaaatagcatcgaagctgagcaaggtgcaggtgagcaatcctctactcaacccctacaaagatgtcttcgaggccaaatatggcctggataaagacgcctctggaatctattctgtgaacattaataagtttaacgacatctttaagaaactgtactcatttactgagttcgaccttgccaccaaattccaggtcaaatgcaggcagacatacattggtcagtacaagtattttaagctttccaatctgctcaatgactcaatttacaacatctccgaaggatacaacataaacaatctgaaagtcaacttccgcggccagaatgccaatctgaacccccggataattacccccatcaccggtaggggcctagtgaaaaagattatcagattttgcaaaaatatcgtttcagtaaaaggtattcggaagagtatatgtattgaaattaataacggggagctatttttcgtagcaagtgaaaattcctacaacgatgacaatatcaacactccaaaagagatcgacgataccgtcacaagcaataacaattacgagaatgatttggatcaggtgattttgaactttaacagcgaaagtgctccaggtctgagcgatgaaaagttgaatctgactattcagaatgacgcctatatccctaagtatgatagcaacggtacaagtgatatcgagcagcacgacgtgaacgaacttaacgtgttcttttacttagacgctcagaaagttcctgagggcgaaaacaatgtgaatttgacctcctcgatagatacagctttgctggaacagcctaaaatttacaccttcttttccagcgagttcattaacaatgtgaataaaccagttcaggctgcgttgtttgtttcttggatacagcaagtccttgtcgactttactaccgaggctaaccagaagagtacggtcgacaaaatagccgacattagcattgtggtcccctacataggactcgctctcaatattggcaacgaagctcagaagggaaactttaaggatgcactggagctgctaggcgcaggtatcctgttagaattcgagccagagctgttgatacccaccattttggtttttactataaagtccttcctgggatcttcggacaacaagaataaagtgatcaaagccatcaataacgctctgaaagaacgagacgagaagtggaaagaggtatactctttcatcgtgtcaaattggatgacaaagatcaacacccagtttaacaaacgaaaggagcagatgtatcaagcgctccagaaccaggttaatgctatcaagactatcattgagtctaagtacaactcctacaccctggaggaaaagaatgagctgactaacaagtacgatatcaagcaaattgagaacgaactgaaccagaaggttagcatcgccatgaacaatattgatcgctttctgaccgagagctcaatcagttacctaatgaagctgatcaatgaagtaaaaatcaacaagctgagagagtacgacgagaatgtgaagacctacctactcaactatatcatacagcacggttccatccttggcgaaagtcagcaagagctgaattccatggttacagatacccttaacaattctatcccgtttaagctaagttcatatacagatgacaaaatactcatttcttatttcaataagttctttaagcgtatcaagagttcctctgtgcttaacatgcgctacaagaacgacaagtatgtcgacacgtccgggtatgacagcaacatcaatattaacggggacgtgtataaatatcccactaacaaaaaccagttcggcatatataatgataaactgtcagaggtgaatatcagtcaaaatgactacattatctatgataacaaatacaagaatttttctatctctttttgggtaaggattccaaattacgacaacaaaatcgtgaatgtgaataacgagtatactatcattaactgcatgagggacaacaatagtggctggaaggtgtcactaaatcacaacgagatcatttggacactgcaggataacgcaggtattaaccagaagcttgcattcaattacggcaatgccaacgggatctccgactacattaataagtggatctttgtcaccataacaaacgaccggctgggtgattctaaattgtatattaatggcaatcttatcgatcagaagtcaatcttaaatctgggcaacattcatgtaagtgacaacatcctcttcaaaatagtgaattgtagctatactcgatatattggcatccgttatttcaacatcttcgataaagaattggacgagacagaaatacaaactctctactccaacgagccaaacacaaacatcctgaaggatttttgggggaactatttactgtatgataaagaatattacctcctgaatgtgcttaagccaaacaatttcattgaccgccgaaaggattccacactgtccatcaataacattcgttccactatcctgttggcgaacagactctactccggcattaaggttaagatccagcgtgtgaacaattccagcaccaatgataacctggtgcgcaagaacgaccaggtgtatatcaacttcgtggcttctaagacacacctttttcccctctacgccgataccgccaccacaaataaagagaagactatcaagatctctagctctgggaacagattcaatcaggtcgttgtcatgaacagcgtcgggaacaattgtacgatgaactttaagaacaataacggaaacaatatcgggctgctcggattcaaggcagacaccgtcgttgccagtacatggtattacacacacatgcgggatcacacaaactctaacggatgtttctggaacttcatttccgaggaacacggttggcaggagaagtga

SEQ ID NO: 83 - DNA, 3759 bp, Artificial Sequence
  mat_peptide (1)...(3756): BoNT/E, S. scrofa-modified 1
atgcccaagattaactcctttaactacaatgatccagttaatgaccgcaccattctgtacatcaagcctgggggctgccaggaattctacaagtcttttaacatcatgaagaacatttggatcattccggagaggaacgtgatcgggactacaccgcaagacttccatcccccaacatccctcaaaaatggggactcaagctactatgaccccaactacctgcagtccgacgaagagaaagatagattcctgaaaatcgtgaccaaaatattcaatagaatcaacaataacttgagtgggggaattctcctagaggaactcagcaaggccaacccctatctgggtaacgataacacccccgacaatcagttccatatcggagatgccagtgcagtggaaatcaaattttctaatgggtcccaggacattttacttccgaacgtgatcataatgggtgccgaaccagatctattcgagacaaactcctctaacatctccctccggaataactacatgcctagcaatcacggcttcgggtccatagcgattgtgaccttctctcccgagtattcattccgattcaacgacaactccatgaacgagtttatccaggaccctgcacttacattgatgcacgaacttatccatagtctccacggcctgtatggagccaagggaattactaccaaatacaccataacccagaagcagaacccactgattactaacatccgaggcacgaacatcgaagagtttctgacctttgggggcaccgaccttaacatcataacatcagctcaatcaaacgacatctacacaaacctgctcgccgactacaaaaagatagctagcaaactctctaaggtgcaagtaagtaatccgttactcaacccctacaaggatgtcttcgaggccaagtatgggctggataaggacgcgtctgggatctactctgtcaatatcaacaagttcaacgatatcttcaaaaagctttactccttcaccgagtttgacctggcaacaaaattccaggtgaaatgcaggcagacctacatcggccagtacaagtactttaaactgagcaacctgttgaatgacagcatctataacatcagtgaagggtacaacattaacaatctgaaagtgaattttcggggacaaaacgctaatctcaaccctagaatcattactcccatcaccggtcgtggcctcgtaaagaaaattataaggttctgtaagaatatcgtgtccgtcaagggcatccgcaagtctatatgtatagagatcaataacggcgagttattctttgtagcgagcgagaactcgtacaatgacgataacatcaacaccccaaaggagatcgacgataccgtgacttcgaataacaattatgagaatgatctggaccaagtgatcttaaacttcaacagtgaatcagcccctggtctaagcgatgagaaactgaatttaaccatccagaatgatgcctatatccccaagtatgacagcaatggcacgagcgatattgagcagcacgatgttaacgaactaaatgtgttcttttaccttgacgctcagaaagtccctgagggcgaaaacaatgtgaatctgacctctagcattgacaccgcgctcctggagcaacctaaaatttatacgttcttttcctcagagttcattaacaatgtaaataagcccgtccaggccgctctgtttgtgtcctggatccagcaagtcctggtggacttcacaaccgaggcaaatcagaaatcaacagtcgataaaatcgccgacatctccatagtggttccttatatcggactcgccctgaatattggtaatgaggcccagaagggtaacttcaaggacgctcttgaattgctcggcgctggcatcctgttagagtttgagccagagctgctcatcccgaccattctggtttttactataaagtcgttcctggggagctccgataacaaaaacaaggtcatcaaggccataaataacgcactcaaagagagggacgaaaagtggaaggaggtgtattcctttatcgtgtccaactggatgactaagatcaatacgcagttcaacaagcgcaaggagcagatgtaccaggccctgcaaaaccaagtcaacgcaattaagactatcattgagtctaaatacaactcctataccttggaggaaaaaaacgaactcactaacaagtatgatatcaagcaaattgagaacgaactgaaccagaaagtttcaattgccatgaataacatcgataggttcctgacagaaagttccataagctatctcatgaaactgatcaacgaagtcaagattaacaagctgcgggagtatgacgagaacgtcaaaacgtacctactgaattatatcatacagcacggctcaattcttggcgaaagccagcaggagttgaatagtatggtcaccgacaccttgaataacagtattccctttaagctgtcgtcttacaccgacgataagatcctgatctcctactttaacaaattctttaaacgaatcaagagtagctccgtcttgaacatgagatacaagaacgataaatacgtggacacgtctgggtacgacagtaatatcaacatcaatggtgatgtgtacaagtacccaactaataagaaccagtttggaatttataacgacaagctcagcgaagtgaatatttcacagaacgattatatcatttacgacaataagtacaagaacttcagcatcagcttctgggtcagaatccccaattacgataacaagattgtaaacgttaataacgagtacaccatcataaattgcatgcgggacaataactcggggtggaaggtgtctctgaaccacaatgaaatcatttggacccttcaggacaacgctggcatcaatcagaagctggcctttaattacggaaatgctaacggaatctcagattacatcaataagtggatcttcgtcacaattacgaacgaccgcctgggggactctaagctgtatattaacggtaatctaatcgatcagaaatccatcctgaaccttggcaacatccatgtgtccgacaatatcctcttcaaaatcgtgaactgctcctacacacggtatatcggaattaggtatttcaatatcttcgacaaagagctggatgagaccgagatacagacactgtacagcaatgagcctaacacgaacattctgaaggacttctggggcaactatctgttgtacgataaggagtactatctccttaacgtcctgaagccaaacaatttcattgaccgcagaaaggactccactctgagcattaataacatccgtagtaccatcctgctcgccaatcgcctctactctggcattaaggttaaaatccagagggtgaataacagctctacaaacgacaatttggttcggaagaacgatcaggtgtacattaacttcgtggcaagcaagactcatctctttcccttgtacgccgacacagcgaccactaacaaggagaagacaatcaaaatctccagctcgggcaatcggtttaaccaggtggtcgtgatgaatagcgtgggcaacaattgcacgatgaatttcaaaaacaacaacggaaacaacatcggactgctggggttcaaggccgacacggttgtggcttcaacctggtactacactcacatgcgcgaccacaccaacagcaacggatgtttttggaacttcataagcgaagagcacggctggcaggaaaaatga

SEQ ID NO: 84 - DNA, 3759 bp, Artificial Sequence
  mat_peptide (1)...(3756): BoNT/E, S. scrofa-modified 2
atgcctaaaatcaactcctttaactacaacgaccccgtaaacgaccgaaccatcttgtatataaaacccggcgggtgccaggagttctacaagagcttcaacattatgaaaaatatctggattatcccggagcggaacgtcattgggactacaccccaggacttccatcccccaacctccttaaagaatggagattcctcttattacgacccgaactatctgcagagcgatgaggaaaaggacaggttccttaagattgtgaccaagattttcaatcggatcaacaataacttatctgggggcatactcctggaagagttaagtaaagccaacccatatctcgggaatgataatacccctgacaaccaattccatattggcgacgcgtccgccgttgagatcaagttttcgaacggatcccaagatatactgctcccaaacgtgatcattatgggcgcggaacctgatctgttcgagactaactccagcaatatttccttgcgcaacaattacatgccctctaatcacggtttcggctcaatcgctatcgtcactttcagccccgagtacagttttcgcttcaacgataactccatgaacgagtttatccaagacccagccttaacactgatgcacgagctgatccacagcctgcacggcctgtacggcgctaagggcattacgacaaaatacaccatcacccaaaaacagaatccactcattactaacatcaggggcaccaacatcgaggaatttctgactttcgggggcacggatctcaacatcataacctcggctcagagtaatgatatctacactaacctgctcgccgactacaagaaaattgcatccaagctttctaaggtgcaggtgtccaacccgctgctaaacccttataaggacgtcttcgaggccaagtacggccttgacaaagacgctagcgggatctatagcgtgaatatcaataagtttaatgacatcttcaagaaactttactcattcacagagttcgacctggccaccaaattccaagtgaagtgccgacagacgtatatcgggcagtacaaatatttcaaactgagtaatctgttgaacgattcaatttacaacatctccgaaggatacaacattaataacctgaaagtgaactttcgcggccagaatgcaaacctgaatccacgaattatcactcccataacaggcaggggcctcgtaaaaaagatcatacgcttttgtaaaaacatcgtgtccgttaaaggtatccggaagtccatttgcattgagatcaataacggagagctattctttgtggcgagcgagaactcatacaatgacgataatatcaacaccccaaaagagattgatgacaccgtcacatctaacaataactatgagaatgatcttgaccaggttattctcaatttcaactccgagtcagctccgggcctaagtgacgagaagctgaacctgacaatccagaacgatgcctacattcccaagtatgattccaatggtacatctgacattgagcagcacgacgtgaacgagctcaacgtgttcttttacctcgacgcccagaaggtccctgagggtgagaataacgtcaatttaacctcgtctatcgacaccgctctgctcgaacagcctaagatctacacgtttttcagtagcgaatttatcaacaatgtgaataagccagttcaagctgccctgttcgtgagctggatccagcaagtgctcgtggatttcaccacggaggccaatcagaaaagtaccgtggacaagatagcagacatctcaatcgtcgtgccttatatcggcctcgccctgaacattgggaacgaggcccagaagggcaattttaaagacgcactggaattgctgggggctgggatcctcctggagtttgaacctgagctgcttatacccacaatcctggtgtttactattaaatcttttcttgggagctctgataacaagaacaaggtgataaaggccatcaacaatgcactcaaggagagggatgaaaaatggaaagaagtgtactcgttcatcgttagtaattggatgaccaagatcaacacgcagttcaataaacgcaaggagcagatgtaccaggccctccagaatcaggtcaacgccatcaaaaccatcatagagagcaaatacaacagctataccctggaggaaaagaatgaactgactaacaagtacgacatcaagcagatcgagaacgaacttaatcagaaagtcagcatagctatgaacaatatcgacagatttctgacagaaagtagcattagctatctcatgaagctgatcaatgaagttaagatcaacaagctcagggaatacgatgaaaatgtgaagacttacttgctgaactacatcattcagcatggatctatcctcggagaaagccagcaagagctgaattctatggtgacggacacactgaataactccatccccttcaagttgtcaagctacaccgatgacaagattcttatctcctacttcaataaatttttcaagcggatcaagagctcgagcgttctgaatatgcggtataaaaacgataagtatgtagatacgtccggatatgacagcaacatcaatattaatggggacgtgtacaagtaccccacaaacaagaaccaattcggcatttacaacgacaagctgtcggaagtgaacatctcacagaatgactacattatatacgacaacaaatacaaaaatttttcaatctcattttgggtccgcatccccaactacgataataagatcgtgaacgtgaacaatgagtataccattataaattgtatgagagacaacaatagcggatggaaggtctcccttaatcacaacgagatcatatggacgctccaggacaatgccggtatcaaccagaagttggcgtttaactatggtaacgccaatggaatctcagactatattaacaagtggatctttgtgacaatcaccaacgataggctgggtgactctaagctgtacattaacggaaaccttatcgaccaaaagtctatattgaatttggggaacatccacgtgagtgataacattctgttcaagattgtgaactgctcctacaccagatacatcggcatccgttacttcaacattttcgacaaagagctcgatgagaccgaaattcagaccttgtacagcaatgaacccaacacgaatatcctgaaagatttctggggcaactacctgctatacgacaaggagtattacctgctcaacgtgctgaagcctaacaatttcatcgaccgcagaaaggattctacactgagcattaacaatatcagaagcactattctactcgcaaacaggttgtatagtggaatcaaggtcaaaatacagcgtgtcaacaattcctcaaccaatgacaacctggttcggaaaaacgatcaggtttatatcaacttcgtagcaagcaaaactcacctatttccgttatatgccgacaccgccacaaccaacaaggagaagactatcaagatctcttcctctggaaaccggttcaaccaggtcgtagtgatgaacagtgtcggcaacaattgcactatgaatttcaaaaacaataacggtaacaacatagggctgctggggttcaaggctgacaccgtcgtcgcgtccacctggtactatacccatatgagagatcacacaaactccaacggatgtttctggaactttatttccgaagagcatggctggcaggagaagtga

SEQ ID NO: 85 - DNA, 3759 bp, Artificial Sequence
  mat_peptide (1)...(3756): BoNT/E, S. scrofa-modified 3
atgcccaaaatcaatagctttaattacaatgaccccgtgaatgacaggactatcctgtacatcaagccaggcggttgccaggagttctacaagagctttaatatcatgaaaaacatctggatcattccggagcggaacgtgatcggtactaccccgcaggactttcacccacccacaagtctgaaaaatggcgactcctcatattacgatccgaactacctgcagtccgacgaagagaaggatcgcttcctgaaaatcgtcacaaagatcttcaatagaatcaacaataacctttccggcgggatcctgctcgaagagctgtccaaggccaacccttacttggggaacgataatacgccagacaatcagttccatattggcgacgccagcgccgtcgaaattaaattcagtaatggcagccaggacatcctgcttcccaacgtgatcattatgggtgcagagcctgatttgttcgaaaccaactctagcaatatctctctgcggaacaattacatgcctagcaatcatggcttcggaagcatcgccatcgtgacctttagcccagagtacagcttcagattcaatgacaactcaatgaatgagtttatccaggatccggccctgaccctgatgcatgaactcattcactcactccacggactctacggggctaagggtattaccacgaagtacaccatcactcagaagcagaatcccctgatcaccaacattaggggcactaacatcgaggaatttctgacctttggagggacagatctgaatatcattacatccgcccagtctaacgatatctacaccaatctcctggccgattataagaaaatcgccagcaagttgagcaaagtgcaggtttccaaccctctcctgaatccttataaagacgtgtttgaggccaagtacgggctggacaaagatgccagtggcatctatagcgtcaatattaataagttcaatgacatcttcaagaaactttactcattcaccgaattcgacctcgcaactaagtttcaggtgaagtgcagacagacctacatcggacagtacaaatacttcaaactgagcaacctgctcaacgattccatttacaatatctccgaaggctataatattaacaatctcaaggttaactttcggggacagaacgctaacctgaacccccggatcattacacccatcaccggtcggggcctggtcaagaaaattatccgattctgtaagaacatcgtctccgttaaagggatccggaaatcaatttgcatcgagatcaacaatggcgagctgttctttgtggcaagcgagaactcctacaacgacgataatatcaacacacctaaggaaatcgatgacacggtgaccagcaataacaattatgagaatgatttggaccaggttatcttgaacttcaatagcgagtccgccccaggcctgtccgatgagaagctgaacctgaccatccagaacgatgcctacatccccaagtacgactcaaatggcacctctgacattgaacagcacgacgtgaacgagctgaacgtgttcttttaccttgatgcccagaaagtccccgagggagaaaataacgtgaacctcactagttccatcgacacagctctgctcgagcagcctaagatttacactttctttagtagcgagttcatcaataacgtgaacaagcctgttcaggccgcattgtttgtttcttggatccagcaggtgcttgtcgacttcacaacggaagccaatcagaaatctacagtggataaaatcgctgacattagcattgtcgtgccatacatcggcctggcactgaatatcggaaacgaggcccagaaagggaacttcaaggacgcactggaactgctcggcgcagggatcctgctcgagttcgagcccgaacttctgattccaaccatcctcgtgttcactattaagagctttcttggatcaagtgataataagaacaaggtgatcaaggctatcaacaatgccctgaaagagagagatgagaagtggaaggaagtgtacagcttcattgtctctaattggatgaccaagatcaacacccagttcaacaagagaaaagaacagatgtaccaggctctgcagaaccaggtgaatgccatcaaaaccatcattgagagcaaatataacagctataccctcgaagagaaaaacgagctgacgaacaagtatgacatcaagcagatcgagaatgaactgaatcagaaggtgtctatcgctatgaacaatattgaccgattcctgaccgagagcagtatctcttatctgatgaagttgattaacgaagtgaagatcaacaaactgcgggaatacgatgagaacgttaagacttatctgttgaactatatcattcagcacgggtctatcctcggggagtcccagcaggagctgaactccatggtgacagacacactgaacaattcaattcctttcaagctgtccagctatacagatgacaagatcctcatctcttactttaacaagtttttcaagaggattaaaagctctagtgtgctgaatatgaggtacaagaacgacaaatatgtcgatacctctggatacgactccaacatcaacatcaacggtgacgtctataagtatcccaccaataagaaccagttcggcatctacaacgacaagttgtccgaagtgaacatttctcagaacgactatattatctacgacaataagtataaaaatttctccatctctttctgggtccgcattccgaactacgacaacaagattgtgaatgtgaacaatgagtacactatcattaactgcatgcgcgacaacaatagcggctggaaagtgtcccttaaccacaacgagatcatttggaccctccaggacaacgccggcatcaatcagaagctcgcctttaactatggtaacgctaatggaatttccgattatatcaacaaatggatcttcgtcaccatcacgaacgacaggctgggggactcaaagttgtacatcaacgggaaccttatcgatcagaaatcaatcctgaacttgggaaacatccatgtcagcgacaacattctgttcaaaatcgtgaactgtagttatacaaggtatatcggcatccgatattttaacattttcgacaaggagctggatgaaacagagatccagaccctgtactccaatgaacccaataccaacatcctgaaagatttttgggggaactacctgctctacgataaggagtactatctgctcaacgtgcttaagcccaataactttatcgacagacgcaaggattccaccctgagtatcaacaatatccgcagcaccattctgctcgctaacaggctctactcaggcatcaaggtgaagatccagagggtgaacaattcttccacgaacgacaacctggttcgcaagaacgaccaggtttacatcaactttgtggccagtaaaacccacctgttcccactgtacgccgatacggccactacaaataaggagaaaactattaagatcagctccagtggaaacaggttcaaccaggtggtcgtgatgaactcagtcggcaataactgtactatgaattttaagaacaataacggaaacaatatcgggcttctggggttcaaggccgacacggtggtcgctagcacgtggtactacactcacatgcgcgaccatacaaatagcaacggctgcttctggaactttatcagcgaggagcacggttggcaggagaaatga

SEQ ID NO: 86 - DNA, 3759 bp, Artificial Sequence
  mat_peptide (1)...(3756): BoNT/E, B. tarsus-modified 1
atgcccaaaataaactcctttaactacaacgatccagtgaacgacaggactatactgtacatcaagcccggcgggtgccaagagttttataagtcatttaatatcatgaaaaacatctggataatccctgagagaaatgtgattggaacaactccccaggattttcaccccccaacctcactaaaaaatggtgacagcagttattacgatcccaactacctgcagtctgacgaggaaaaggatcggttcttaaaaatcgtcaccaaaatctttaaccggataaataacaatctctctggcgggatcctgcttgaggaactgagcaaagctaatccttatctcggaaacgacaacacaccagataatcagtttcacattggggacgcatctgctgtggagattaagttctccaacggcagccaggatatcctgctccccaacgtcatcataatgggagccgagcctgacttgtttgagacgaactccagtaacataagccttagaaacaattacatgccctcgaaccacggatttggttcaatcgccatagtgaccttcagtccagaatacagcttccgctttaatgataactcaatgaacgaattcattcaggacccagctctgacattgatgcatgagctgattcactccctccacggtctgtacggagccaagggcataactacaaagtataccatcacccaaaagcagaacccgctcattaccaatatccgcggaacgaacatcgaggaatttctcacctttggtggaaccgatctgaacattataacaagtgcccagtcgaacgacatctacaccaatttgctggccgactataaaaagatcgctagcaagctgtcaaaggtgcaggtgagcaaccccctcttgaatccttacaaggatgtgtttgaagctaaatacgggttagacaaggacgcttccggaatctacagcgtgaacatcaacaagttcaacgatatctttaagaaactgtactctttcaccgagtttgatctggcaactaaatttcaggtcaagtgccgccagacttacatcggtcaatataagtattttaaactcagcaacctgcttaacgacagcatttacaatatcagcgaagggtataacatcaacaatctgaaggtcaatttccgagggcagaacgcaaacctgaatccaaggatcattacccctatcacaggccgtggcctggtcaagaaaattatcaggttctgtaagaacatcgtctctgtaaagggcatccgaaagtccatctgtatcgaaattaataacggggagctgtttttcgttgccagcgaaaacagctacaacgatgacaacatcaacacgcccaaggaaattgacgataccgtcacttccaacaataactatgagaacgatctcgatcaggttatcctgaatttcaatagcgagtcagcaccagggctaagtgatgagaagctcaatctgactatacagaacgatgcgtacattcccaaatacgatagcaacggcaccagcgacattgaacagcatgatgttaatgagctcaacgtgttcttttatctggacgcccagaaagttccggagggtgagaacaatgtcaatctgacttcctctatcgatacagccctgcttgagcagcctaagatctacactttctttagctcggaattcatcaacaatgtgaataagccggttcaggccgcactgttcgtctcttggatacagcaagtgctggtggacttcaccactgaggccaatcagaagtctacggtcgacaagattgctgacatctctatcgtagttccttatattggcctcgccctcaacatcggcaacgaggcacagaagggcaacttcaaagatgccctggagcttctgggtgctggaattctgcttgagttcgaaccagagctcctgatccctaccatccttgtattcaccatcaagtcctttctcggcagctctgataataagaacaaggtcatcaaggccattaataacgcgctgaaagagagggacgagaaatggaaagaggtgtactccttcattgtcagcaattggatgaccaagattaatacacagttcaacaaaaggaaggagcagatgtatcaggcactccagaaccaggtgaatgcaataaagaccataatcgagtccaaatataattcgtacactcttgaagagaaaaacgaacttacgaacaagtatgacattaaacagatagagaacgagctgaatcagaaagtctcaattgcgatgaacaatatcgaccgtttcctgacagagagctccataagctacctcatgaaactaatcaatgaggtgaagatcaacaagttgcgggagtatgacgaaaacgtaaagacataccttttgaattatatcattcaacatggcagtatcttaggcgaaagccagcaagaattgaactcaatggtgaccgacaccttgaacaatagtattccgttcaagctcagttcctacacagacgataagatactgatttcatatttcaacaagttctttaaacgaattaagtccagttctgtgctgaatatgcggtacaagaacgacaaatacgtggacacctctggctacgactctaatatcaacattaacggggatgtgtataaatatcctaccaacaagaaccagtttggtatctataacgacaagttgtccgaagtgaatatcagtcagaacgattacattatctacgataacaagtacaagaatttctccatctccttttgggtgcggatacccaactacgacaataagatcgtgaacgtgaataacgaatacacaatcattaactgcatgagagacaataactcgggatggaaggtttccctcaaccacaatgagattatctggacactgcaggacaacgctggcattaaccaaaaattggccttcaactatgggaatgcgaacgggattagcgactacatcaataagtggattttcgtaactatcactaacgatcggctcggcgacagtaagctgtatatcaatggaaacctgattgaccaaaaatctattttaaacctaggtaacatccatgtctcggacaacatcctcttcaagatcgtgaactgttcttacacaagatatattgggatccgatacttcaatattttcgataaggagctcgacgagaccgaaattcaaacactgtacagcaacgaacctaacaccaatatccttaaagatttttgggggaactacttactttacgacaaagagtactatttactaaatgtgctgaagcccaacaattttatagatcgcagaaaagacagtacgctgagcatcaacaatatccgttccacaatcctgctagccaacaggctgtactcaggcattaaggttaaaatccagagggtgaataactcctcaaccaacgacaatctggtcagaaaaaatgaccaggtgtacattaatttcgtggctagtaagactcacttgttcccactgtatgccgacactgccacgacaaataaggaaaaaacgatcaaaatcagttccagtggcaaccgctttaaccaagtcgtggttatgaattctgtgggaaacaattgcaccatgaacttcaagaacaataacggaaacaatatcggtttgctcggcttcaaggccgacacagtagtggcttcaacctggtattacacccacatgcgcgaccacacgaactctaacggatgcttctggaatttcatttccgaagagcatgggtggcaggaaaaatga

SEQ ID NO: 87 - DNA, 3759 bp, Artificial Sequence
  mat_peptide (1)...(3756): BoNT/E, B. tarsus-modified 2
atgcccaagatcaacagcttcaactacaacgaccccgtgaacgacaggaccatcctgtacatcaagcccggcggctgccaggagttctacaagagcttcaacatcatgaagaacatctggatcatccccgagaggaacgtgatcggcaccaccccccaggacttccacccccccaccagcctgaagaacggcgacagcagctactacgaccccaactacctgcagagcgacgaggagaaggacaggttcctgaagatcgtgaccaagatcttcaacaggatcaacaacaacctgagcggcggcatcctgctggaggagctgagcaaggccaacccctacctgggcaacgacaacacccccgacaaccagttccacatcggcgacgccagcgccgtggagatcaagttcagcaacggcagccaggacatcctgctgcccaacgtgatcatcatgggcgccgagcccgacctgttcgagaccaacagcagcaacatcagcctgaggaacaactacatgcccagcaaccacggcttcggcagcatcgccatcgtgaccttcagccccgagtacagcttcaggttcaacgacaacagcatgaacgagttcatccaggaccccgccctgaccctgatgcacgagctgatccacagcctgcacggcctgtacggcgccaagggcatcaccaccaagtacaccatcacccagaagcagaaccccctgatcaccaacatcaggggcaccaacatcgaggagttcctgaccttcggcggcaccgacctgaacatcatcaccagcgcccagagcaacgacatctacaccaacctgctggccgactacaagaagatcgccagcaagctgagcaaggtgcaggtgagcaaccccctgctgaacccctacaaggacgtgttcgaggccaagtacggcctggacaaggacgccagcggcatctacagcgtgaacatcaacaagttcaacgacatcttcaagaagctgtacagcttcaccgagttcgacctggccaccaagttccaggtgaagtgcaggcagacctacatcggccagtacaagtacttcaagctgagcaacctgctgaacgacagcatctacaacatcagcgagggctacaacatcaacaacctgaaggtgaacttcaggggccagaacgccaacctgaaccccaggatcatcacccccatcaccggcaggggcctggtgaagaagatcatcaggttctgcaagaacatcgtgagcgtgaagggcatcaggaagagcatctgcatcgagatcaacaacggcgagctgttcttcgtggccagcgagaacagctacaacgacgacaacatcaacacccccaaggagatcgacgacaccgtgaccagcaacaacaactacgagaacgacctggaccaggtgatcctgaacttcaacagcgagagcgcccccggcctgagcgacgagaagctgaacctgaccatccagaacgacgcctacatccccaagtacgacagcaacggcaccagcgacatcgagcagcacgacgtgaacgagctgaacgtgttcttctacctggacgcccagaaggtgcccgagggcgagaacaacgtgaacctgaccagcagcatcgacaccgccctgctggagcagcccaagatctacaccttcttcagcagcgagttcatcaacaacgtgaacaagcccgtgcaggccgccctgttcgtgagctggatccagcaggtgctggtggacttcaccaccgaggccaaccagaagagcaccgtggacaagatcgccgacatcagcatcgtggtgccctacatcggcctggccctgaacatcggcaacgaggcccagaagggcaacttcaaggacgccctggagctgctgggcgccggcatcctgctggagttcgagcccgagctgctgatccccaccatcctggtgttcaccatcaagagcttcctgggcagcagcgacaacaagaacaaggtgatcaaggccatcaacaacgccctgaaggagagggacgagaagtggaaggaggtgtacagcttcatcgtgagcaactggatgaccaagatcaacacccagttcaacaagaggaaggagcagatgtaccaggccctgcagaaccaggtgaacgccatcaagaccatcatcgagagcaagtacaacagctacaccctggaggagaagaacgagctgaccaacaagtacgacatcaagcagatcgagaacgagctgaaccagaaggtgagcatcgccatgaacaacatcgacaggttcctgaccgagagcagcatcagctacctgatgaagctgatcaacgaggtgaagatcaacaagctgagggagtacgacgagaacgtgaagacctacctgctgaactacatcatccagcacggcagcatcctgggcgagagccagcaggagctgaacagcatggtgaccgacaccctgaacaacagcatccccttcaagctgagcagctacaccgacgacaagatcctgatcagctacttcaacaagttcttcaagaggatcaagagcagcagcgtgctgaacatgaggtacaagaacgacaagtacgtggacaccagcggctacgacagcaacatcaacatcaacggcgacgtgtacaagtaccccaccaacaagaaccagttcggcatctacaacgacaagctgagcgaggtgaacatcagccagaacgactacatcatctacgacaacaagtacaagaacttcagcatcagcttctgggtgaggatccccaactacgacaacaagatcgtgaacgtgaacaacgagtacaccatcatcaactgcatgagggacaacaacagcggctggaaggtgagcctgaaccacaacgagatcatctggaccctgcaggacaacgccggcatcaaccagaagctggccttcaactacggcaacgccaacggcatcagcgactacatcaacaagtggatcttcgtgaccatcaccaacgacaggctgggcgacagcaagctgtacatcaacggcaacctgatcgaccagaagagcatcctgaacctgggcaacatccacgtgagcgacaacatcctgttcaagatcgtgaactgcagctacaccaggtacatcggcatcaggtacttcaacatcttcgacaaggagctggacgagaccgagatccagaccctgtacagcaacgagcccaacaccaacatcctgaaggacttctggggcaactacctgctgtacgacaaggagtactacctgctgaacgtgctgaagcccaacaacttcatcgacaggaggaaggacagcaccctgagcatcaacaacatcaggagcaccatcctgctggccaacaggctgtacagcggcatcaaggtgaagatccagagggtgaacaacagcagcaccaacgacaacctggtgaggaagaacgaccaggtgtacatcaacttcgtggccagcaagacccacctgttccccctgtacgccgacaccgccaccaccaacaaggagaagaccatcaagatcagcagcagcggcaacaggttcaaccaggtggtggtgatgaacagcgtgggcaacaactgcaccatgaacttcaagaacaacaacggcaacaacatcggcctgctgggcttcaaggccgacaccgtggtggccagcacctggtactacacccacatgagggaccacaccaacagcaacggctgcttctggaacttcatcagcgaggagcacggctggcaggagaagtga

SEQ ID NO: 88 - DNA, 3759 bp, Artificial Sequence
  mat_peptide (1)...(3756): BoNT/E, B. tarsus-modified 3
atgcctaagatcaactcttttaattataacgatccagtaaatgacagaacaatcctgtacattaagcccggtgggtgccaggaattttacaagagttttaacattatgaaaaacatttggattataccagaacgcaacgttatcggcaccacaccccaggactttcaccctccgacttcgctgaaaaacggtgatagctcttattacgaccccaactatctgcagtccgacgaggaaaaagacagatttctgaagattgtcactaagatcttcaacagaatcaataacaatctgtctgggggaatcctcctggaggaactttcaaaggccaacccttacttgggtaacgacaacactcccgataatcaattccatataggcgacgcctctgctgtggagataaagttctcaaacggaagtcaggacatcctgcttcctaacgtaatcataatgggagccgaaccagatctcttcgagaccaatagctcaaacatcagtcttaggaataactacatgcctagcaaccacgggtttggctctattgccatagtgactttctcgcccgagtattcctttcgatttaatgataacagcatgaacgagttcatccaagatcccgcacttaccctgatgcacgagctgattcactctctgcacgggctctatggagccaaaggcattacaaccaagtacaccatcactcaaaaacagaacccacttatcacaaatatcaggggcacaaacatcgaagagtttttgaccttcggaggcacagacctgaacattatcacctccgctcaatcaaacgacatctacaccaatctcctggccgactacaagaaaatcgcatcaaagctcagcaaggttcaggtttccaatcctctgttgaatccatataaggatgtcttcgaagcaaaatacggcctagacaaggacgccagtggaatttacagtgtgaatatcaataagttcaatgatatcttcaaaaagctgtactcctttaccgagtttgacttagcgacgaagttccaagtaaaatgcaggcagacatacatcggccagtacaaatatttcaagctgtccaatcttttaaacgactcgatttataatatcagtgagggctacaatattaacaatttgaaagtaaatttccgggggcagaacgctaacctgaacccgcgaattatcacgcccataaccgggcggggtctggtgaagaaaattatacgcttttgcaaaaacatcgtgagcgtgaaggggattaggaaaagcatctgtatcgaaatcaacaatggggagctcttctttgtggcctctgagaactcgtataatgatgacaatatcaacacacccaaggagattgacgatactgtgacctctaacaataactacgagaatgacctagaccaggtgatcctcaactttaacagtgaaagtgcccccggccttagtgatgagaagttgaacttaaccattcagaatgacgcgtatataccgaagtatgacagcaatggtacgagtgatatcgaacagcatgacgtgaatgaattgaacgtgtttttctacctggatgctcaaaaagtgcccgagggcgaaaacaatgtcaatcttaccagctccattgacacagcactgctcgagcaaccaaagatttacacctttttctcctctgagtttattaacaatgtgaacaagcctgtccaggctgccctcttcgttagttggatccagcaagtgctggtggacttcacaacggaagctaaccagaaatcgaccgtggataaaattgccgacatctccatcgtcgtgccttacattggactcgctctgaacatcgggaatgaagcacagaagggcaactttaaagatgctttagagcttctgggagccgggatcctcctggagttcgaacccgagctactgatccccactatcctcgtcttcaccatcaaatcctttctgggttcctctgacaataagaataaggtcataaaggcaatcaataacgctttgaaagagcgggatgagaagtggaaagaggtctatagcttcatagtcagcaactggatgactaagattaatacccagttcaacaaacggaaggagcaaatgtaccaggccctccagaatcaagtcaatgccatcaagaccatcatagagagcaagtacaactcctatactttggaagagaagaatgagctcaccaacaaatacgacatcaaacagatcgagaacgaactgaaccagaaggtgtcaatcgctatgaacaatatcgaccgtttcctgacagagtcatccatctcatacttgatgaagctgattaacgaggtgaagatcaataagctgcgtgagtacgatgaaaacgtcaaaacatatttgctaaactatataattcagcacggatccattttaggtgagagccagcaggaactgaactctatggttaccgacaccttgaacaatagcataccattcaagctgtctagctatacagatgacaaaatactgatcagctacttcaataaattctttaaaagaatcaagtccagcagtgtgctgaatatgcgctacaagaacgataaatacgtggatacctccggatacgattcaaacattaacatcaatggcgacgtatacaagtacccaactaataagaaccagtttggaatttataatgataaacttagcgaagtgaacatctcccagaacgactacatcatttacgataacaagtataagaacttttcgatctccttttgggtcaggattcctaattacgacaacaaaatagttaacgtcaacaatgagtacacgatcattaactgcatgcgagacaataactccggctggaaggtgtcactgaaccataatgaaatcatttggacgctccaggataacgccgggatcaaccagaaacttgcgttcaactacggaaacgccaatggtatttccgactatattaacaagtggattttcgtgacgatcacgaatgacagactcggtgactctaaactgtacatcaacggcaacctcatcgaccagaagagcattcttaacctgggcaatattcatgtttccgataacatcctgttcaagatcgtgaactgttcttacacacgctacattgggatccgatactttaacattttcgataaagagctggatgagaccgaaatccagaccctgtacagtaacgaaccgaacaccaacatcttaaaagacttctggggtaactatctactgtatgataaggaatactatctgctcaacgtcctcaagccaaacaatttcatagacaggagaaaagacagcactctgtcaatcaacaatatccgtagcacgatcttgctcgccaatcgcctctactctggcataaaggtgaagatccagcgggtgaacaattctagcactaacgataacctggtccggaagaatgatcaggtttatattaatttcgtggcttccaagacacatctgtttcctctctatgccgacaccgcgactaccaacaaggagaaaacaatcaagataagctctagcgggaatcgcttcaaccaggttgtagtgatgaactcagtcggaaataactgcactatgaacttcaagaataacaatggcaacaacattggcctcctaggcttcaaggcagacacagtggtggcaagtacttggtattatacacacatgagggaccacaccaacagtaacggatgtttctggaactttatcagcgaggaacacgggtggcaggagaagtga

SEQ ID NO: 89 - DNA, 3759 bp, Artificial Sequence
  mat_peptide (1)...(3756): BoNT/E, E. caballus-modified 1
atgcccaagataaactctttcaattacaacgatcccgttaatgacagaaccatactgtacatcaagcctggaggctgccaggagttttacaaaagcttcaacataatgaagaacatctggatcattcctgagaggaatgtaatagggacaaccccgcaagacttccacccccctactagtcttaaaaacggtgactcaagttactatgatcccaactacttgcagagcgacgaggaaaaggacaggttccttaaaattgtcacaaagatattcaataggatcaataacaatctctccggcggtattctactcgaggaactgtcaaaggccaacccttacctgggcaatgataacacccctgacaatcagtttcatatcggcgatgccagcgccgtcgaaatcaagttcagtaacggcagccaggatattctgctccctaacgtgatcattatgggcgcagaacctgacttattcgagacaaatagttctaacatctcactgagaaataactacatgcctagtaatcacggtttcggctccatagcaattgtgactttctcccctgagtatagctttcgctttaacgacaactcaatgaatgagtttattcaagacccagccctaactctgatgcacgaactgatccattcgttacacggattatatggcgctaaaggaatcactacgaagtatactattacccagaaacagaacccactaatcaccaacataaggggcactaacatcgaagagtttcttaccttcgggggaaccgatttgaatattatcacctccgcccagtcaaacgacatttacacaaacctgttggccgactataagaaaatcgcgtccaaactgtccaaggtccaggtgagtaatccactgttgaacccatacaaagatgtcttcgaagctaaatacggattggataaggacgcctccggcatatacagcgtgaatattaataagtttaacgatatttttaaaaagctctactccttcactgagttcgatctggccactaagtttcaggtgaagtgccggcagacctacatagggcaatataaatatttcaagctctcaaatctcctgaacgatagtatctacaacatcagcgagggatataacatcaataacctgaaggtgaactttcgcgggcagaacgccaacctaaatccgcgaatcattactccaatcacaggtagaggactggttaaaaagataatccggttctgcaagaacattgtcagcgtgaagggaatcaggaaaagcatttgtatcgagatcaataacggagaattatttttcgtggcatcagaaaacagctacaatgatgacaacatcaacacccccaaggagatcgatgacactgtgacgtctaacaataactacgagaatgatttggatcaggtcatcttaaacttcaacagcgagtcagcccccgggctcagcgacgagaaattgaacctgaccatacaaaatgacgcctacatacccaaatatgattcaaacggcacctctgacatcgagcagcatgatgtgaatgagctgaacgtcttcttttatctggacgcccaaaaggtcccagaaggagagaacaatgtcaatctcacttctagtatcgataccgccctgctcgaacagccgaaaatttataccttctttagctccgaattcatcaataacgtcaacaagcccgtgcaggctgcacttttcgtgagttggattcagcaagtgctcgtagactttaccactgaggccaatcagaaatccaccgttgataaaattgctgatatctctatcgtggtcccctacatcggcctggctcttaacataggcaacgaggcacagaaagggaacttcaaggacgcgctggagctgctcggagccgggatcctgctcgaattcgagccagaactgttaataccgacgatccttgtattcacaattaagtcatttctcggctcctctgacaataaaaataaggtgatcaaagccatcaataacgcactcaaggagagagatgagaagtggaaggaagtctactcgtttatcgtgtccaactggatgaccaagattaacacacagtttaacaagcgcaaggaacagatgtaccaggctctgcagaaccaggtcaacgctattaagactatcattgagtcaaagtacaacagctacaccctggaggaaaagaacgaactcacgaacaagtacgatatcaagcaaattgagaatgagctgaatcaaaaggtttccatcgctatgaacaatatagaccggttcctcaccgaatcctctatttcctatctgatgaagttgattaatgaagttaagattaacaagctgcgggagtatgacgagaacgtgaagacatatctgcttaattatattatccaacatgggagtattctgggggaatcacaacaggagctgaattctatggtaacagacaccctgaacaatagtatcccatttaagctcagctcctatacagatgacaagattcttatctcttactttaacaagtttttcaagcgtatcaagagcagttcagttctaaacatgcgctacaagaacgacaagtatgtggacacaagtggttatgactcgaacatcaatatcaacggcgacgtgtacaaataccccacgaacaagaaccagttcggcatttacaacgacaaactgagcgaggtgaatatcagccagaatgactacattatctatgacaataaatataaaaacttctccattagcttttgggttagaatccccaattatgataataaaatagtgaacgttaacaatgagtacaccatcattaattgcatgagggataacaattctgggtggaaggtgtctttgaatcacaacgagatcatttggactctgcaggacaacgcaggaatcaaccagaagctggctttcaattatgggaatgctaatggcatatctgactacattaacaaatggatcttcgtgacaatcaccaacgacagactgggggattctaaactctacatcaacgggaaccttatcgatcagaagtcgattctgaaccttggaaacatccacgtgtccgacaacatactgttcaagatcgtgaattgtagctacacgcgttacatcggcatcaggtacttcaatatcttcgacaaagagctcgacgagaccgagatccagacgctctactccaatgaacctaacaccaatatcctgaaggacttctggggaaactacttgctgtatgacaaggagtactatctcttgaatgtgctgaaacccaacaatttcatcgaccgacggaaagacagcacgctctctatcaataacatccgctctaccattctgctagcgaaccgtctgtactccggcatcaaagtaaagatccagcgggtgaataacagtagcacaaacgataacctggtcagaaaaaacgatcaggtgtacatcaacttcgtcgccagcaaaacacatctttttcctctttatgcggacactgcgactaccaataaagaaaagaccattaaaatctcctcttccggcaaccgatttaatcaagtggtcgtgatgaatagcgtgggtaataactgtactatgaattttaagaataacaatggtaataacattggtttgctggggtttaaggcagatacggttgtagcctcaacatggtactacacacacatgcgcgaccacaccaattccaatggctgtttctggaactttatctcggaagagcatggttggcaggagaaatga

SEQ ID NO: 90 - DNA, 3759 bp, Artificial Sequence
  mat_peptide (1)...(3756): BoNT/E, E. caballus-modified 2
atgcccaagatcaacagcttcaactacaacgaccccgtgaacgacagaaccatcctgtacatcaagcccggcggctgtcaggagttctacaagagcttcaacatcatgaagaacatctggatcatccccgagagaaacgtgatcggcaccaccccccaggacttccacccccccaccagcctgaagaacggcgacagcagctactacgaccccaactacctgcagagcgacgaggagaaggacagattcctgaagatcgtgaccaagatcttcaacagaatcaacaacaacctgagcggcggcatcctgctggaggagctgagcaaggccaacccctacctgggcaacgacaacacccccgacaaccagttccacatcggcgacgccagcgccgtggagatcaagttcagcaacggcagccaggacatcctgctgcccaacgtgatcatcatgggcgccgagcccgacctgttcgagaccaacagcagcaacatcagcctgagaaacaactacatgcccagcaaccacggcttcggcagcatcgccatcgtgaccttcagccccgagtacagcttcagattcaacgacaacagcatgaacgagttcatccaggaccccgccctgaccctgatgcacgagctgatccacagcctgcacggcctgtacggcgccaagggcatcaccaccaagtacaccatcacccagaagcagaaccccctgatcaccaacatcagaggcaccaacatcgaggagttcctgaccttcggcggcaccgacctgaacatcatcaccagcgcccagagcaacgacatctacaccaacctgctggccgactacaagaagatcgccagcaagctgagcaaggtgcaggtgagcaaccccctgctgaacccctacaaggacgtgttcgaggccaagtacggcctggacaaggacgccagcggcatctacagcgtgaacatcaacaagttcaacgacatcttcaagaagctgtacagcttcaccgagttcgacctggccaccaagttccaggtgaagtgtagacagacctacatcggccagtacaagtacttcaagctgagcaacctgctgaacgacagcatctacaacatcagcgagggctacaacatcaacaacctgaaggtgaacttcagaggccagaacgccaacctgaaccccagaatcatcacccccatcaccggcagaggcctggtgaagaagatcatcagattctgtaagaacatcgtgagcgtgaagggcatcagaaagagcatctgtatcgagatcaacaacggcgagctgttcttcgtggccagcgagaacagctacaacgacgacaacatcaacacccccaaggagatcgacgacaccgtgaccagcaacaacaactacgagaacgacctggaccaggtgatcctgaacttcaacagcgagagcgcccccggcctgagcgacgagaagctgaacctgaccatccagaacgacgcctacatccccaagtacgacagcaacggcaccagcgacatcgagcagcacgacgtgaacgagctgaacgtgttcttctacctggacgcccagaaggtgcccgagggcgagaacaacgtgaacctgaccagcagcatcgacaccgccctgctggagcagcccaagatctacaccttcttcagcagcgagttcatcaacaacgtgaacaagcccgtgcaggccgccctgttcgtgagctggatccagcaggtgctggtggacttcaccaccgaggccaaccagaagagcaccgtggacaagatcgccgacatcagcatcgtggtgccctacatcggcctggccctgaacatcggcaacgaggcccagaagggcaacttcaaggacgccctggagctgctgggcgccggcatcctgctggagttcgagcccgagctgctgatccccaccatcctggtgttcaccatcaagagcttcctgggcagcagcgacaacaagaacaaggtgatcaaggccatcaacaacgccctgaaggagagagacgagaagtggaaggaggtgtacagcttcatcgtgagcaactggatgaccaagatcaacacccagttcaacaagagaaaggagcagatgtaccaggccctgcagaaccaggtgaacgccatcaagaccatcatcgagagcaagtacaacagctacaccctggaggagaagaacgagctgaccaacaagtacgacatcaagcagatcgagaacgagctgaaccagaaggtgagcatcgccatgaacaacatcgacagattcctgaccgagagcagcatcagctacctgatgaagctgatcaacgaggtgaagatcaacaagctgagagagtacgacgagaacgtgaagacctacctgctgaactacatcatccagcacggcagcatcctgggcgagagccagcaggagctgaacagcatggtgaccgacaccctgaacaacagcatccccttcaagctgagcagctacaccgacgacaagatcctgatcagctacttcaacaagttcttcaagagaatcaagagcagcagcgtgctgaacatgagatacaagaacgacaagtacgtggacaccagcggctacgacagcaacatcaacatcaacggcgacgtgtacaagtaccccaccaacaagaaccagttcggcatctacaacgacaagctgagcgaggtgaacatcagccagaacgactacatcatctacgacaacaagtacaagaacttcagcatcagcttctgggtgagaatccccaactacgacaacaagatcgtgaacgtgaacaacgagtacaccatcatcaactgtatgagagacaacaacagcggctggaaggtgagcctgaaccacaacgagatcatctggaccctgcaggacaacgccggcatcaaccagaagctggccttcaactacggcaacgccaacggcatcagcgactacatcaacaagtggatcttcgtgaccatcaccaacgacagactgggcgacagcaagctgtacatcaacggcaacctgatcgaccagaagagcatcctgaacctgggcaacatccacgtgagcgacaacatcctgttcaagatcgtgaactgtagctacaccagatacatcggcatcagatacttcaacatcttcgacaaggagctggacgagaccgagatccagaccctgtacagcaacgagcccaacaccaacatcctgaaggacttctggggcaactacctgctgtacgacaaggagtactacctgctgaacgtgctgaagcccaacaacttcatcgacagaagaaaggacagcaccctgagcatcaacaacatcagaagcaccatcctgctggccaacagactgtacagcggcatcaaggtgaagatccagagagtgaacaacagcagcaccaacgacaacctggtgagaaagaacgaccaggtgtacatcaacttcgtggccagcaagacccacctgttccccctgtacgccgacaccgccaccaccaacaaggagaagaccatcaagatcagcagcagcggcaacagattcaaccaggtggtggtgatgaacagcgtgggcaacaactgtaccatgaacttcaagaacaacaacggcaacaacatcggcctgctgggcttcaaggccgacaccgtggtggccagcacctggtactacacccacatgagagaccacaccaacagcaacggctgtttctggaacttcatcagcgaggagcacggctggcaggagaagtga

SEQ ID NO: 91 - DNA, 3759 bp, Artificial Sequence
  mat_peptide (1)...(3756): BoNT/E, E. caballus-modified 3
atgcccaagataaactcctttaactataacgatcccgtgaacgaccgaacgatattgtacattaagccaggcgggtgtcaggagttctacaaatcattcaacataatgaaaaacatctggattatccccgagagaaacgtgattggcactacacctcaggacttccatcccccaacgagtcttaagaacggagacagctcttattacgaccccaattacctgcaatcggacgaggaaaaagatagatttctgaagatcgtgacgaagatttttaatcggatcaataacaatttatctggagggatcctcctggaggaacttagtaaggcaaatccatatttggggaacgataacacccctgataaccaattccatatcggcgatgccagtgccgtggagattaagttcagtaacggatcccaggatatcctcttgcccaacgtaatcattatgggtgcggagccagacctgttcgagactaacagttctaacatttcactgagaaacaattacatgccttccaatcacggatttgggagcattgccatcgttaccttctcgcccgaatattccttccgatttaacgacaatagtatgaacgagttcattcaggaccccgctttgactctcatgcatgagcttatccactctctgcacggactctacggcgctaagggtattaccactaagtacaccatcacccagaagcagaaccccctgattacaaatatacggggaacaaatattgaggaattcctgacgttcgggggcacagatctcaacatcattaccagcgctcagagcaatgacatttataccaatctgctcgcagattacaaaaagatagcctccaagctgtctaaggtccaggtgtccaatccgcttctaaatccttataaggatgtcttcgaggccaagtacggtctggacaaagacgccagcggcatttatagcgtgaacattaacaagtttaacgacatcttcaagaaactctactccttcaccgagtttgaccttgctacaaaattccaggtgaagtgtagacagacatacatcgggcaatataagtattttaagttaagcaatcttctgaacgactcaatttacaacatctccgaggggtacaatattaacaatctgaaggtgaactttcgcggccaaaacgcgaatcttaaccctcgtatcataactccgattaccggtcgcggcctggtgaaaaagataatcaggttctgcaaaaacatcgtgtctgtgaagggcatccgaaaatccatttgcatcgagattaacaatggcgaattgtttttcgtggcgagtgaaaattcttataacgacgataatatcaacactcctaaagaaatcgacgatactgttacatctaacaataactacgagaatgacctcgaccaggtcatcctgaacttcaactccgagagtgccccaggactctccgatgaaaaactcaacctgaccatccagaacgatgcatacatccctaaatatgattctaacggcacaagtgacatcgagcaacacgatgtgaacgagctgaatgtgtttttctacctagatgcgcagaaagtccccgagggggaaaacaatgtgaacttgacctcttcaatcgacacggcactcttagagcagcccaaaatctacaccttctttagctcagagtttatcaacaatgttaacaagcccgtccaggccgcattattcgtcagctggattcaacaggtactggtcgattttaccacagaggccaaccagaagtctacggtggacaaaattgccgacatctccatcgtcgtaccatacatcggcttggcactgaacatcgggaacgaggcccaaaaaggtaacttcaaggatgcgttggagttgttaggtgcaggaatcctgctagaatttgaaccggaactcctgatccctaccatactcgtcttcactatcaaatctttcctagggtcatccgacaacaagaataaggtgataaaggccatcaataacgctctgaaagagcgtgacgagaaatggaaagaggtgtacagcttcatagtctcgaactggatgaccaaaattaacacgcaattcaacaagaggaaagaacagatgtatcaggccctgcagaaccaggtaaacgccataaagacaataatcgaatccaaatacaattcctacaccctcgaagagaagaacgagctgactaacaagtacgacatcaaacagatcgagaatgaactgaatcaaaaggtgagcatcgctatgaacaatattgatcggtttctgaccgaatcttccatctcctacctgatgaagctcatcaatgaggttaagataaataaactgcgggagtatgacgagaacgtgaagacgtacctgctcaattatatcattcagcatggatcaatcctcggcgagtcccagcaagaactgaactcaatggtaaccgacactcttaataacagcataccgttcaagctcagctcatacaccgacgataaaatcttgatcagttattttaacaagtttttcaagcgcattaagagctcatccgtccttaatatgagatacaaaaatgacaaatacgtggacacaagtgggtacgactccaacatcaatattaatggtgacgtttataagtatcctacaaataagaaccagtttgggatctataatgacaagctctccgaagtcaatatatcacagaacgactacatcatttacgacaataaatataaaaacttctcgatttcattttgggtgcgcatcccaaactacgataataagatcgttaacgtgaataacgagtataccattataaactgtatgcgcgataacaatagcggatggaaggtgagcctcaatcacaacgagatcatttggacactgcaggataatgccggtattaatcagaagctggccttcaactatggaaacgctaacgggattagcgactacatcaataagtggatctttgtgacaataaccaacgaccggcttggagacagtaagctgtatattaatggcaatctgatcgaccagaaatctatcctgaatctgggcaacattcatgtcagcgataatatcctattcaaaatagtgaactgttcttacacgagatacattggtatcaggtacttcaacatcttcgataaggaactggacgagactgaaatccagaccttgtacagcaatgaacctaatactaatatcctgaaggacttttggggcaactacctactttacgataaggaatactatctcttaaacgtgctcaaacctaataactttatcgataggcgtaaggacagcacactgtcaatcaataacatcaggagtaccatcctgttggctaatagactgtatagcggcatcaaagtgaagatccagcgcgtcaacaattcatcgactaacgacaacctggtgaggaaaaacgatcaggtttacatcaacttcgtcgccagtaagactcatctgttcccactgtacgcagatactgctaccactaataaggagaaaaccattaagatctccagctctgggaataggtttaatcaggtggttgtaatgaacagcgtgggcaataactgcaccatgaactttaagaacaataacggcaataacattggacttctgggatttaaggctgataccgtcgttgcctccacttggtactatacacacatgcgggaccacaccaacagcaatggctgcttctggaatttcatctctgaggaacacggctggcaagagaagtga

SEQ ID NO: 92 - DNA, 3759 bp, Artificial Sequence
  mat_peptide (1)...(3756): BoNT/E, C. aethiops-modified 1
atgccaaaaattaatagcttcaactataatgacccggtaaacgaccgtaccatcttgtatatcaaacccgggggctgccaggagttttacaagagttttaatatcatgaaaaacatctggatcattcctgaacggaacgtgattgggacaaccccgcaggacttccaccccccaacaagtcttaagaacggtgacagttcctattacgatcccaactacctgcagtcagatgaagagaaagatagattcctgaagatcgttacaaagatctttaataggatcaacaataacttatctggcgggatactcctggaggaactatccaaggccaatccatacttggggaacgacaatacccccgacaatcaatttcacattggtgatgcgagtgctgtggagataaagtttagtaatggaagccaagacatactactgcctaatgtgatcataatgggggcggaaccggatctgttcgagactaactctagcaacatcagcttgagaaacaattacatgccttctaatcacggctttgggtccattgctatcgtgactttctcgccggaatactcatttcgctttaacgacaattcaatgaacgagtttatacaggatccagccttaactctgatgcatgaacttatccactcgctgcatggtctgtacggagcaaaaggcattacaaccaaatacacgatcacacaaaagcaaaacccactcattaccaacatccgtgggacgaacattgaggaattcctcacattcggcggaacggatctgaatatcataacaagtgcccagtcgaatgatatctataccaatcttctggctgattacaagaaaatcgcaagcaagctctccaaggtgcaagtctctaaccctttacttaacccttataaggacgtctttgaagccaagtacggactcgacaaggatgcttccggcatttattctgtcaacatcaataaattcaacgatatctttaaaaagctgtattcctttacagagttcgatctggccaccaagttccaggttaaatgccggcagacctacatcggccagtacaagtatttcaagcttagtaatcttttgaatgacagtatttacaacatcagcgagggctataacattaacaatctgaaggtgaacttcagaggacaaaatgctaaccttaacccaaggatcattacaccaatcacgggcagaggcttggtgaagaaaattatcagattttgcaaaaacatcgtcagtgtgaaagggatccggaagtcaatttgcatcgaaatcaataacggcgagctgttctttgtggcttccgaaaactcgtataacgacgataacattaacacacccaaggagatcgacgatacggtcactagcaacaataactatgagaatgatctggatcaggtgattctgaatttcaacagtgaaagcgcccccggcctctctgatgagaaattgaatctcacgatccagaacgacgcctacatcccaaagtatgattccaacgggactagcgacatagaacagcatgacgtgaatgagctcaatgtgttcttttatctggatgcccagaaagtacctgagggggaaaataacgttaatcttacttcttcaattgatacggccctcctggaacagcccaagatttataccttcttttcctctgagttcatcaataacgtgaacaagcctgtccaggcggccctgttcgtctcttggattcagcaagtgctcgtcgacttcaccacagaagcaaaccagaagagcaccgttgataagatagctgatatctctattgtggtaccctacataggcttggcgctgaatattggaaatgaggcccaaaaaggaaacttcaaagacgcactggagctgttgggggcaggcatcctactcgaattcgagcctgagttgctgatccctactatcctggttttcacaattaaaagttttctgggttcttcagacaacaagaacaaagtgatcaaagcaatcaataacgccctgaaggaacgagacgagaaatggaaagaagtgtatagcttcattgtttccaattggatgaccaaaataaacacccagtttaacaaaaggaaagaacagatgtaccaggctctgcagaatcaggttaatgccattaaaactattatcgagtctaaatataacagttataccctggaggaaaagaacgagttgaccaataaatacgacatcaagcaaatcgagaacgagctgaaccagaaggtttctatcgcaatgaataacatagatcgctttcttactgagagctccattagttatctcatgaagctaatcaacgaggtcaaaatcaacaaactgagggagtatgatgagaatgtgaagacttacttgctgaattacataatccagcatggctccattctgggtgagtcccagcaagaactaaattccatggtaacggacaccctgaacaattccatcccattcaagcttagtagctacacagacgataagattcttattagctactttaataaattctttaagcggatcaagtcctcaagcgttctcaacatgcgatacaaaaacgataagtacgtagacacatccggatacgactcaaacattaatataaacggtgacgtgtataagtaccccacgaacaagaaccagtttggaatctataatgataaacttagcgaagtgaacatctctcaaaacgactacatcatttatgacaataaatacaaaaatttctcaatctcattttgggtacggattcccaactatgataacaaaatcgtcaatgtgaacaatgaatacactatcattaattgtatgcgagataataacagcggctggaaagtgagcctcaaccacaacgagataatttggaccctgcaagacaacgcaggaatcaaccaaaagttagcttttaattatggcaacgccaacggtatttctgactacatcaataaatggatattcgttaccataacaaacgaccgcctcggagactccaagctgtacatcaatggaaacctcattgaccagaagagcatactcaatctggggaacattcatgtgagcgacaacatccttttcaagatcgtcaattgctcatacacaagatacataggtatccgttacttcaacattttcgataaggagttagacgagacggaaatccagactctttattccaatgagccaaacactaacatcttaaaagacttctggggaaattacctcttgtatgacaaagaatattacttacttaacgtcctgaagcccaacaatttcatcgaccgccggaaggattccaccctgtctattaataacatcagatctactattctcctggccaatcgcctttattctggcataaaggtcaaaattcagcgagtgaataactcatcgacgaacgataacctcgttaggaagaacgaccaggtgtatatcaacttcgtggcttctaagacgcatctatttccactgtacgctgataccgctactacaaacaaggagaagaccatcaagattagctcaagcggaaatcgctttaaccaggtcgtggtcatgaattccgttggcaacaattgtacaatgaattttaagaacaataacgggaataatattggtttgctagggttcaaagccgacaccgtcgtcgcaagcacttggtattatacacacatgagggatcacacaaattctaatgggtgtttctggaatttcatctcagaggaacacggctggcaggagaaatga

SEQ ID NO: 93 - DNA, 3759 bp, Artificial Sequence
  mat_peptide (1)...(3756): BoNT/E, C. aethiops-modified 2
atgccaaagatcaacagcttcaactacaacgacccagtgaacgacagaaccatcctgtacatcaagccaggcggctgccaggagttctacaagagcttcaacatcatgaagaacatctggatcatcccagagagaaacgtgatcggcaccaccccacaggacttccacccaccaaccagcctgaagaacggcgacagcagctactacgacccaaactacctgcagagcgacgaggagaaggacagattcctgaagatcgtgaccaagatcttcaacagaatcaacaacaacctgagcggcggcatcctgctggaggagctgagcaaggccaacccatacctgggcaacgacaacaccccagacaaccagttccacatcggcgacgccagcgccgtggagatcaagttcagcaacggcagccaggacatcctgctgccaaacgtgatcatcatgggcgccgagccagacctgttcgagaccaacagcagcaacatcagcctgagaaacaactacatgccaagcaaccacggcttcggcagcatcgccatcgtgaccttcagcccagagtacagcttcagattcaacgacaacagcatgaacgagttcatccaggacccagccctgaccctgatgcacgagctgatccacagcctgcacggcctgtacggcgccaagggcatcaccaccaagtacaccatcacccagaagcagaacccactgatcaccaacatcagaggcaccaacatcgaggagttcctgaccttcggcggcaccgacctgaacatcatcaccagcgcccagagcaacgacatctacaccaacctgctggccgactacaagaagatcgccagcaagctgagcaaggtgcaggtgagcaacccactgctgaacccatacaaggacgtgttcgaggccaagtacggcctggacaaggacgccagcggcatctacagcgtgaacatcaacaagttcaacgacatcttcaagaagctgtacagcttcaccgagttcgacctggccaccaagttccaggtgaagtgcagacagacctacatcggccagtacaagtacttcaagctgagcaacctgctgaacgacagcatctacaacatcagcgagggctacaacatcaacaacctgaaggtgaacttcagaggccagaacgccaacctgaacccaagaatcatcaccccaatcaccggcagaggcctggtgaagaagatcatcagattctgcaagaacatcgtgagcgtgaagggcatcagaaagagcatctgcatcgagatcaacaacggcgagctgttcttcgtggccagcgagaacagctacaacgacgacaacatcaacaccccaaaggagatcgacgacaccgtgaccagcaacaacaactacgagaacgacctggaccaggtgatcctgaacttcaacagcgagagcgccccaggcctgagcgacgagaagctgaacctgaccatccagaacgacgcctacatcccaaagtacgacagcaacggcaccagcgacatcgagcagcacgacgtgaacgagctgaacgtgttcttctacctggacgcccagaaggtgccagagggcgagaacaacgtgaacctgaccagcagcatcgacaccgccctgctggagcagccaaagatctacaccttcttcagcagcgagttcatcaacaacgtgaacaagccagtgcaggccgccctgttcgtgagctggatccagcaggtgctggtggacttcaccaccgaggccaaccagaagagcaccgtggacaagatcgccgacatcagcatcgtggtgccatacatcggcctggccctgaacatcggcaacgaggcccagaagggcaacttcaaggacgccctggagctgctgggcgccggcatcctgctggagttcgagccagagctgctgatcccaaccatcctggtgttcaccatcaagagcttcctgggcagcagcgacaacaagaacaaggtgatcaaggccatcaacaacgccctgaaggagagagacgagaagtggaaggaggtgtacagcttcatcgtgagcaactggatgaccaagatcaacacccagttcaacaagagaaaggagcagatgtaccaggccctgcagaaccaggtgaacgccatcaagaccatcatcgagagcaagtacaacagctacaccctggaggagaagaacgagctgaccaacaagtacgacatcaagcagatcgagaacgagctgaaccagaaggtgagcatcgccatgaacaacatcgacagattcctgaccgagagcagcatcagctacctgatgaagctgatcaacgaggtgaagatcaacaagctgagagagtacgacgagaacgtgaagacctacctgctgaactacatcatccagcacggcagcatcctgggcgagagccagcaggagctgaacagcatggtgaccgacaccctgaacaacagcatcccattcaagctgagcagctacaccgacgacaagatcctgatcagctacttcaacaagttcttcaagagaatcaagagcagcagcgtgctgaacatgagatacaagaacgacaagtacgtggacaccagcggctacgacagcaacatcaacatcaacggcgacgtgtacaagtacccaaccaacaagaaccagttcggcatctacaacgacaagctgagcgaggtgaacatcagccagaacgactacatcatctacgacaacaagtacaagaacttcagcatcagcttctgggtgagaatcccaaactacgacaacaagatcgtgaacgtgaacaacgagtacaccatcatcaactgcatgagagacaacaacagcggctggaaggtgagcctgaaccacaacgagatcatctggaccctgcaggacaacgccggcatcaaccagaagctggccttcaactacggcaacgccaacggcatcagcgactacatcaacaagtggatcttcgtgaccatcaccaacgacagactgggcgacagcaagctgtacatcaacggcaacctgatcgaccagaagagcatcctgaacctgggcaacatccacgtgagcgacaacatcctgttcaagatcgtgaactgcagctacaccagatacatcggcatcagatacttcaacatcttcgacaaggagctggacgagaccgagatccagaccctgtacagcaacgagccaaacaccaacatcctgaaggacttctggggcaactacctgctgtacgacaaggagtactacctgctgaacgtgctgaagccaaacaacttcatcgacagaagaaaggacagcaccctgagcatcaacaacatcagaagcaccatcctgctggccaacagactgtacagcggcatcaaggtgaagatccagagagtgaacaacagcagcaccaacgacaacctggtgagaaagaacgaccaggtgtacatcaacttcgtggccagcaagacccacctgttcccactgtacgccgacaccgccaccaccaacaaggagaagaccatcaagatcagcagcagcggcaacagattcaaccaggtggtggtgatgaacagcgtgggcaacaactgcaccatgaacttcaagaacaacaacggcaacaacatcggcctgctgggcttcaaggccgacaccgtggtggccagcacctggtactacacccacatgagagaccacaccaacagcaacggctgcttctggaacttcatcagcgaggagcacggctggcaggagaagtga

SEQ ID NO: 94 - DNA, 3759 bp, Artificial Sequence
  mat_peptide (1)...(3756): BoNT/E, C. aethiops-modified 3
atgccaaaaattaactcatttaattacaacgatccggtgaatgacaggacaattttgtacattaagccaggcgggtgccaggagttctacaaatccttcaacattatgaagaacatctggattatccccgaaagaaatgtgattggcacgacaccacaggacttccacccacctacttccctgaaaaacggggatagttcttactatgatcccaactatctgcagtctgacgaagagaaagacagatttttgaagatagtgacaaagatttttaaccgaattaataacaatctgtccggcggaatacttctagaggaactgtcaaaagccaacccctatttggggaatgataatactcccgacaaccagttccacatcggtgatgcctctgctgtagagattaagttttctaacggcagccaagatattctgctccccaatgtcattatcatgggcgcagaacctgacctgttcgagacaaacagcagtaatatctctctcagaaataactatatgccaagtaaccacggctttgggtcaattgctatcgttacgttctcccctgaatattcatttcgattcaatgacaatagcatgaacgagttcatacaagatcccgctcttacgctgatgcacgagctgatccactcactccatggtctgtatggggccaaaggtataactacgaagtacaccataacccaaaagcagaaccccctcataacgaacatccgaggcaccaacatcgaggaattcctgaccttcgggggcaccgatctgaacatcattacgagcgctcagagcaacgacatttacacaaacctcctggccgattataaaaagatcgcgagcaagctaagtaaggtccaggtgtccaatccccttttaaatccttacaaagatgtgttcgaagccaagtacggcttggataaagatgcgtcaggcatttacagcgttaacataaacaagttcaatgatatcttcaagaaactttatagttttacagagtttgaccttgctactaaattccaggttaaatgtaggcaaacttacatcggccagtataaatacttcaaactgtccaatctgttgaatgattcaatttacaatatcagcgaaggttacaacataaacaatttgaaagtgaatttcaggggccaaaatgcaaacttgaatccaaggatcataactccaattaccgggcggggcctggttaaaaagattatccgattttgcaagaatatcgtgtctgtgaaaggaattagaaaatctatatgcatcgagatcaacaatggcgagttatttttcgtggcaagcgagaactcttataacgacgataatattaacacccctaaggagattgatgacaccgtgacgtcgaataacaattacgagaatgatttggaccaggtgatccttaattttaactccgagtctgccccaggacttagtgacgagaagctgaacctaacaatccagaatgacgcatatattcccaagtatgattcaaatggaacatctgacatcgagcagcacgatgtaaatgagctaaacgttttcttttacctggacgcccagaaggttccggagggcgagaacaatgtcaatctgactagctccatcgacactgctctcctggaacaacccaaaatctatacttttttctcaagtgagttcatcaacaatgttaataagcctgtccaggccgcactcttcgtctcgtggattcagcaagttctcgttgacttcactacagaagcaaaccagaagtcgaccgtcgacaagattgccgacattagcatcgtagtcccttacatagggctggcgctgaatatcggaaacgaggcccagaaggggaactttaaagacgcgctagaactgctcggggccggaatactcttggagttcgagcccgaactcctgataccgaccatcctggtgtttacaatcaagagcttcctgggtagctccgataataagaataaggtgatcaaggctattaataacgcacttaaggaacgggacgagaagtggaaagaggtttacagcttcattgtgagtaactggatgacaaaaatcaacactcagtttaacaagcgtaaagagcaaatgtatcaggcactccagaaccaggtaaatgcaattaaaactatcattgaaagcaagtataatagctatacactggaggaaaagaatgagctgaccaacaagtacgacatcaaacaaatagaaaacgaattgaaccagaaagtctctatcgccatgaacaatattgaccggttcttaaccgaatcctctattagctacctcatgaagctcatcaatgaggtcaagatcaataagctgcgtgagtacgatgagaacgtcaaaacatatttactgaactatatcattcagcatggttcaatcctcggcgaatcccagcaagaactgaattctatggtcacggacactcttaataactccatacctttcaagctcagcagttacaccgatgacaaaatcctcatctcctactttaacaaatttttcaagcgcattaagagtagctctgtgctgaatatgagatataagaatgacaagtacgtggatacctctgggtacgattctaacataaatatcaatggagacgtgtacaagtaccctacaaataagaatcagtttggtatctataatgataaactcagtgaggttaacatctcccaaaacgattacattatctatgataacaaatacaagaattttagcatctccttttgggtacggatcccgaattatgacaataaaattgttaacgtaaacaatgaatatacgatcattaactgcatgcgcgataataactcgggatggaaggtgtctcttaaccacaatgagatcatttggactctgcaggacaatgctggcattaaccaaaaactggcttttaactatggaaacgctaacggaatctcagattatattaacaaatggatctttgtgactatcaccaacgatcggctgggagacagcaagctatacattaatggcaatttaatcgatcaaaagagcatcctaaatttgggaaacatccatgtcagtgacaacatccttttcaaaatcgtgaactgttcgtataccaggtacatcgggatccgctactttaacattttcgataaagaactggacgagacggagatacagacactttactccaacgaaccaaataccaacatcctcaaggacttttgggggaattatcttctctatgacaaagaatattaccttttaaacgtgctgaagccaaacaatttcatcgaccgtagaaaggacagcacactgagtataaataacatccgctctaccatcttgctggccaaccgcctttactccggtatcaaagtgaagattcagagagtcaacaattcatccacaaacgacaacttagtgcgcaaaaacgaccaggtgtatatcaatttcgtggcctccaaaacacacttgtttccactgtatgctgataccgccacgaccaacaaggagaagaccataaagatctcctcatctggaaacaggttcaaccaggtcgtggtcatgaacagtgtggggaacaattgcaccatgaatttcaaaaacaacaacggtaacaacatcggcctgcttggctttaaagctgatacagtcgtcgcctccacttggtactacacgcatatgcgggaccatacaaactctaacggatgtttctggaacttcatttcagaagagcatgggtggcaggaaaaatga

SEQ ID NO: 95 - DNA, 3759 bp, Artificial Sequence
  mat_peptide (1)...(3756): BoNT/E, H. sapiens-modified 1
atgccaaagataaacagctttaattacaacgatcccgtgaacgaccgtaccatcctgtatatcaagcccggcgggtgtcaagagttctataagagctttaatatcatgaaaaacatttggatcatacccgaaaggaacgtgatcggaacaaccccccaagatttccatcccccgacaagcctgaagaacggagatagcagctattacgatcccaactatctgcagagcgacgaggaaaaggatcggtttctgaagatagttacgaagattttcaataggattaacaataacctgagcggagggattctgctggaggaactgagcaaggcaaacccatatctgggtaatgataacaccccggataaccagttccatatcggtgatgccagcgcagtcgaaataaaatttagcaatggcagccaggatatcctgctgccaaatgtaatcattatgggggcagagcctgacctgttcgaaacgaacagcagcaacattagcctgcggaataactacatgccaagcaatcacggatttggtagcattgcgattgtcacctttagccctgaatatagcttcagatttaatgacaacagcatgaacgagtttatccaggatcctgctctgactctgatgcacgagctgatccatagcctgcacggcctgtacggcgccaaaggtatcacgacaaagtacaccattacacagaaacagaatcctctgatcaccaacatccgcgggaccaacatcgaggaattcctgacattcggcgggaccgatctgaatattatcactagcgctcagagcaatgacatctacaccaatctgctggctgactacaaaaagattgccagcaagctgagcaaagtgcaggtgagcaacccactgctgaacccctataaggatgtgtttgaggccaaatacgggctggacaaagacgccagcggcatttatagcgtgaacatcaacaaattcaacgatattttcaagaaactgtacagctttacggagtttgacctggcaacaaaatttcaggtcaagtgtcggcagacgtacataggccagtacaaatattttaagctgagcaatctgctgaatgatagcatctacaatatcagcgagggctacaatatcaataacctgaaagttaattttcgcggccaaaatgcaaacctgaaccctcggattatcacccctatcacagggcgcggtctggttaagaaaattatcaggttttgcaagaacatcgttagcgttaagggcattagaaaaagcatttgtatagagatcaacaatggagagctgttctttgtggctagcgaaaatagctataatgacgataacatcaacaccccaaaggagatagacgataccgtcactagcaataacaattacgagaatgacctggaccaagtcatcctgaattttaacagcgagagcgctccaggcctgagcgacgagaagctgaacctgaccatccaaaacgatgcctatatccctaagtatgatagcaatggcactagcgatattgaacagcacgacgtaaacgaactgaatgtctttttctatctggacgcacaaaaagtgccagaaggagagaacaatgtgaatctgactagcagcatcgatactgccctgctggagcagcctaagatctacaccttctttagcagcgagttcattaacaatgttaacaagcccgttcaggccgctctgttcgtgagctggattcagcaagtgctggtcgactttacaactgaggctaatcaaaagagcacggttgataaaatagcagatatcagcattgttgtgccgtatatcggactggctctgaacatcggcaatgaggcccagaaaggaaacttcaaggacgccctggaactgctgggggcgggcatcctgctggagttcgaacccgaactgctgatcccaacgattctggtgttcactattaagagctttctgggaagcagcgacaacaagaacaaggtcattaaggcaataaataacgccctgaaagaaagggatgaaaagtggaaggaggtgtacagcttcattgtcagcaactggatgaccaagatcaatacacagttcaataagcggaaagaacagatgtaccaggccctgcagaaccaggtgaatgcgattaagactattatcgagagcaaatacaacagctacaccctggaagagaagaacgaactgactaataaatatgacattaagcagattgagaacgagctgaaccagaaagtgagcatcgcaatgaataacatagaccgattcctgacagagagcagcataagctatctgatgaaactgatcaacgaggtgaaaatcaataagctgcgtgaatatgacgagaacgtgaagacttatctgctgaactatatcatacagcacggcagcattctgggagaaagccaacaggagctgaacagcatggtgaccgataccctgaataacagcataccctttaagctgagcagctacactgacgataaaatactgataagctactttaacaaattctttaagagaataaaaagcagcagcgttctgaatatgcgctacaaaaatgataagtatgtggatacaagcggttacgacagcaatattaatattaatggcgacgtgtataagtacccgacaaacaaaaatcaattcggtatctataatgacaaactgagcgaagtgaacatcagccagaacgactatatcatatatgacaataaatacaagaatttcagcatcagcttctgggtgagaatacctaactacgacaataagattgtaaacgtgaataacgagtacacaatcattaactgcatgagagacaacaatagcgggtggaaagtcagcctgaaccataatgagattatctggactctgcaagacaacgccggaatcaatcagaaactggccttcaactacggaaacgctaatggtattagcgattatatcaataaatggatatttgtaaccatcacaaatgatcgactgggggacagcaagctgtacattaacggtaacctgatcgaccagaagagcatactgaacctgggaaacatccacgtaagcgataacatcctgtttaaaattgtcaactgcagctacactaggtatatcgggatcagatacttcaatattttcgacaaagaactggatgagacagaaattcagaccctgtacagcaacgagccaaacaccaacatcctgaaagacttctggggcaattatctgctgtacgacaaggaatactatctgctgaacgtactgaagcctaataactttatcgacaggcgtaaggacagcactctgagcatcaataacattaggagcactattctgctggctaaccgcctgtacagcggcattaaagtaaagattcagcgggtgaacaatagcagcacaaatgacaatctggtgcgaaaaaacgatcaggtctatattaatttcgtcgccagcaagacacatctgttccccctgtacgccgacacagctacaaccaataaggaaaaaaccattaagataagcagcagcgggaaccgcttcaatcaggtcgttgtgatgaacagcgtcgggaataactgtacaatgaattttaaaaataacaatggaaataacatcggcctgctgggctttaaggccgataccgtggtggcgagcacttggtactacacgcatatgagagatcacaccaatagcaatggttgcttctggaatttcatcagcgaggaacacgggtggcaggagaagtga

SEQ ID NO: 96 - DNA, 3759 bp, Artificial Sequence
  mat_peptide (1)...(3756): BoNT/E, H. sapiens-modified 2
atgccgaagatcaacagctttaactataatgacccagttaacgacagaacaattctgtacattaagcctgggggatgtcaggagttctacaaaagctttaacattatgaagaatatttggataatccccgaaaggaatgtgattgggactacaccacaagacttccatccacccaccagcctgaaaaatggcgatagcagctattacgaccctaattatctgcaaagcgatgaggaaaaagacaggttcctgaagattgtgacaaagatcttcaacagaattaacaataacctgagcggaggcatactgctggaggaactgagcaaggcaaatccctacctgggcaatgacaacacgccagataatcagtttcatatcggagatgccagcgctgtggagatcaaatttagcaacgggagccaggatatcctgctgccaaatgtaattatcatgggtgctgagcctgacctgtttgaaaccaatagcagcaacataagcctgaggaacaattacatgcccagcaaccacgggtttggtagcattgccattgtcaccttcagccctgagtatagctttcgcttcaacgataacagcatgaatgaattcattcaggaccctgctctgaccctgatgcacgagctgatacacagcctgcacgggctgtatggcgctaagggaatcacaaccaaatacactattacacaaaagcagaatccactgataaccaatattcgcgggacaaatatcgaggaattcctgacttttggcggaaccgacctgaacatcataacaagcgcccagagcaatgacatctatactaatctgctggccgattataaaaagattgcgagcaagctgagcaaagtgcaggtgagcaaccctctgctgaatccatataaagacgtcttcgaagctaagtacggtctggacaaagacgctagcggcatctacagcgtgaacattaacaaattcaacgatatctttaaaaagctgtacagctttactgagttcgacctggccaccaagtttcaggttaagtgtcgccagacttacattggacagtacaagtatttcaagctgagcaacctgctgaacgacagcatttataacatcagcgaggggtacaacattaacaatctgaaagtgaacttcagaggccagaacgctaacctgaaccctcgaatcattactccaatcacaggccgcggcctggtgaaaaagattatacgcttctgtaaaaacattgttagcgtaaaaggaatccgtaagagcatatgcattgagatcaacaatggggaactgtttttcgtggccagcgagaatagctacaatgatgacaatatcaacacccccaaagaaatcgatgacaccgtcaccagcaataacaattacgagaacgatctggatcaggtgatcctgaatttcaacagcgagagcgcacccggactgagcgatgaaaagctgaacctgacaatccagaatgacgcatacattccaaaatatgacagcaatggaactagcgatatcgaacagcacgacgtaaatgagctgaacgtcttcttttacctggatgcccagaaggtgccggagggtgagaacaatgtgaatctgactagcagcatcgacaccgctctgctggaacagcccaaaatctacacttttttcagcagcgagttcattaataacgtgaacaagcccgttcaagccgctctgttcgttagctggattcagcaagtgctggtagatttcactacagaggcgaaccagaaaagcaccgttgataagatcgccgacatcagcattgtcgtgccttacatcgggctggccctgaatattggtaacgaggcacagaagggtaattttaaagacgccctggaactgctgggcgccggcatcctgctggagtttgagcccgaactgctgattccgacaatcctggtgttcacaatcaaaagcttcctgggcagcagcgataacaagaacaaggtcataaaagcaatcaataacgctctgaaggaacgggatgagaagtggaaagaagtgtatagcttcatcgtgagcaattggatgaccaaaataaatacacagttcaataagagaaaggagcaaatgtaccaggccctgcaaaaccaggtgaatgccataaaaacaataatcgaaagcaagtataatagctacacactggaagagaagaatgagctgaccaataaatatgacataaaacaaattgagaacgagctgaatcagaaggtcagcatcgcaatgaataacatagacaggttcctgaccgagagcagcatcagctacctgatgaagctgatcaatgaagtgaaaattaataagctgagagagtatgacgagaatgtcaaaacttacctgctgaactatattatccagcacggcagcatcctgggtgaaagccagcaagagctgaatagcatggtgacggatacgctgaataacagcattccctttaagctgagcagctacacagacgataaaattctgatcagctacttcaacaagtttttcaagcgaatcaagagcagcagcgtgctgaacatgcggtacaaaaatgataaatacgtcgacaccagcggctacgacagcaatatcaacatcaatggcgacgtgtacaagtatcccacaaacaaaaatcaatttggcatatacaacgacaagctgagcgaggtgaacattagccagaacgattatattatctacgacaacaagtataaaaattttagcatcagcttttgggtgcgtattcctaattacgataacaagatagttaatgtaaacaatgagtataccattatcaactgcatgagggacaacaatagcgggtggaaggtgagcctgaatcataacgaaattatctggaccctgcaggacaacgcaggaatcaaccagaagctggcatttaactacggcaacgcgaatggtataagcgattatatcaacaaatggatattcgtcactattacgaatgatcgtctgggagatagcaaactgtatatcaatggcaacctgatcgatcagaagagcatcctgaacctgggcaacatccacgtcagcgataatattctgttcaagattgtgaactgcagctacactcggtatatcgggatcaggtattttaacatcttcgataaggaactggacgaaaccgaaatccaaaccctgtatagcaacgagcctaatacgaatatactgaaggacttttgggggaattacctgctgtatgacaaggagtattacctgctgaatgtcctgaagccgaataactttatagatcggcgcaaggatagcacgctgagcatcaataacattcggagcactatcctgctggcaaatcggctgtacagcgggattaaagtgaaaattcagcgagtcaataacagcagcaccaacgataacctggttagaaagaacgaccaggtctacattaattttgttgccagcaaaacacatctgtttccactgtacgccgatactgcgaccacgaataaggaaaagaccatcaagataagcagcagcggaaatagattcaaccaggtagtggtcatgaacagcgttggaaacaattgcacaatgaactttaaaaacaataacggaaataacatcggtctgctggggttcaaagctgacacggtagttgccagcacttggtattatacacatatgagggaccataccaacagcaacggctgtttctggaactttattagcgaagagcacggttggcaggaaaaatga

SEQ ID NO: 97 - DNA, 3759 bp, Artificial Sequence
  mat_peptide (1)...(3756): BoNT/E, H. sapiens-modified 3
atgcctaaaattaacagtttcaattacaacgatccagtgaacgataggaccattctgtacatcaaacccggcgggtgtcaggagttctataaaagctttaatatcatgaaaaacatctggatcataccagaaaggaatgtcattggcactacccctcaggactttcacccacctacttcactcaaaaatggggacagctcatattacgatcctaattacctacaatccgacgaggaaaaggaccgcttcctgaagatcgtgacgaaaatttttaacaggattaacaataacttgtctggcggaatcctgctcgaagagctttccaaagctaatccttatcttggtaatgacaacactccagacaaccagtttcatattggtgatgcttctgctgtcgagataaaattttcgaacggtagtcaagatatcctgctcccaaacgttatcataatgggtgcggaaccggacttgttcgagaccaacagcagtaatatatccctgaggaacaattatatgccgtcaaatcacggctttggatccattgccattgttacatttagccccgagtattcctttcgtttcaacgacaatagcatgaatgagtttatccaggatcccgcactgaccttaatgcatgaacttatacatagccttcatggactgtacggcgccaagggtatcaccacaaaatatacaatcacccagaaacaaaatccccttatcaccaacattaggggcacaaatatagaggaattcttgacatttggtgggacagatcttaacatcataacctccgcccagagcaatgacatttataccaacttgttagcggattacaagaaaattgcctcaaagttgtcaaaggttcaggtgagtaaccccctgcttaacccgtataaggacgtgttcgaggctaagtatggcttagacaaggacgcttctgggatatactctgtgaacatcaataaatttaatgatatttttaagaaactgtattccttcactgaattcgacttggctaccaagttccaggtgaaatgcagacagacttatatcggccagtataagtatttcaagctgagcaatctcctaaacgacagcatctacaatatttctgagggatacaacatcaataacctaaaagtaaatttccgtggccagaatgctaatctcaacccaaggatcattacccccataaccgggagaggcctcgttaagaaaatcattcggttttgtaagaacatcgtcagtgtgaagggaatccgaaaaagcatttgtattgagatcaataacggtgagctgtttttcgtggcctctgagaattcttataatgatgacaacatcaatacgcctaaagaaattgatgacaccgtcacatcgaataacaattacgagaacgatttggaccaggtgatcctaaatttcaactcagaatccgcccctggcctgagtgatgaaaaactgaacctgacaattcagaacgatgcctatattccgaagtatgactccaatgggacgtcggacatcgaacagcacgatgtgaatgaactaaacgttttcttttacctcgacgcccaaaaggtccctgagggcgaaaacaatgttaatctgacatcttccatagatactgctttactggagcagcctaagatttacaccttcttttcgagtgagttcatcaacaatgtcaacaaacccgttcaagccgcactgtttgtcagctggatacagcaagtgctggtggacttcaccacagaagccaatcaaaagtcaacagtggacaagatcgcggatatcagcatcgtggttccctacataggacttgccctgaacatcggaaacgaggcacagaaagggaactttaaagatgcacttgaactgctaggagcagggatccttctcgagtttgagccagagctcctgatccctacgattctggttttcactattaagtctttcctgggcagttccgataacaagaacaaagtcattaaggccattaataacgctctgaaagagcgcgatgaaaagtggaaggaagtgtactctttcatcgtgtccaattggatgactaagatcaatacacagtttaataagcgaaaagagcagatgtaccaggccttgcagaatcaggtgaatgccattaaaaccatcattgaaagcaaatacaatagttacacactcgaagagaaaaacgaactgactaataagtatgatatcaagcaaatcgagaacgagctcaaccagaaggtcagtattgcaatgaacaatattgaccgctttctaacagagagctccattagttacctgatgaaactgatcaatgaggtaaaaataaacaagctgagagaatacgatgagaacgtgaaaacctacctcttgaactacatcattcagcatgggtcaatcctgggagaatctcaacaggagcttaattcgatggtcacagacacccttaacaattccatcccattcaagctttctagttacacggacgataaaatcctgatatcctatttcaacaagttctttaaacggattaagagttccagtgtcttaaacatgagatataaaaatgataagtacgtggacacctctgggtatgactctaatatcaatatcaatggcgatgtctacaagtaccccacaaacaaaaaccagttcggaatctataacgataagctctccgaagtaaacatcagccagaatgactacataatctacgacaacaagtacaagaactttagcatttcattctgggtgcggattcccaattatgacaacaagattgtcaacgtaaataacgagtatacaatcattaattgcatgcgagataataactccggttggaaggtgtccctgaaccacaatgagataatttggactctgcaagacaacgcgggtattaaccagaaactggcatttaactatggaaacgccaatgggattagcgactacatcaataagtggatatttgtgactatcacaaatgaccgcctcggcgatagcaagctgtacatcaatggaaacctgatcgatcagaagtccattctgaatttgggtaacattcacgtatcagacaatattctgtttaagattgtgaactgttcttatactcggtacatcggaatacggtatttcaacatatttgacaaagagctggatgagacagaaatccagaccttgtactccaacgagcccaataccaacatcctgaaggatttctgggggaactacttgctctatgacaaggaatactatttactcaacgttctgaagccaaataacttcattgatagacgcaaggattcaaccctctctattaataacatccgttctaccatccttctcgccaacagattgtactctgggatcaaagtgaaaatacagagagtcaacaatagctcaacgaacgacaatttggttaggaagaacgaccaggtgtatatcaacttcgtggcttctaaaactcacttgtttccactctacgccgatactgctaccacgaataaggagaaaaccataaagatttcaagttctggcaaccgcttcaatcaggtggtcgtaatgaactctgtaggcaataactgcacgatgaatttcaaaaacaataacggcaacaatatcggactactgggctttaaggcagacactgtggtggcaagcacttggtactacactcatatgcgggaccacacaaattccaatggctgcttctggaacttcatcagcgaagagcacgggtggcaagaaaagtga

SEQ ID NO: 98 - DNA, 3811 bp, Artificial Sequence
  mat_peptide (1)...(3811): BoNT/E, E. coli-modified inactive
gatatcggatccgaattcgagctcccatatgcctaaaatcaattcgttcaactataatgacccggttaacgatcgcacgatcctgtatatcaagccaggtggatgtcaagaattttataaatcattcaacatcatgaaaaatatttggattatcccggaacgcaacgtgatcggcacgacgcctcaagattttcacccgccgacctccctgaaaaatggcgacagttcctactatgacccgaattatttacaatcggatgaagaaaaagatcgtttcctcaagatcgtcacgaaaattttcaaccgcatcaataacaatctgtccggtggcatcttacttgaggaattatctaaagctaatccgtatctggggaacgataataccccggataatcagttccacattggcgatgcgagcgctgtggaaattaaattcagcaacggcagtcaagatattcttctcccaaacgtgattatcatgggggctgaacctgatcttttcgaaactaatagttccaatatttcactgcgcaataattatatgccgtcgaaccatcgctttggctcaatcgcaattgtgacgttctcacctgaatatagttttcgttttaacgacaactgcatgaatgaatttatccaagacccggcgctgactttgatgcatgaactgatccatagcttgcacggcctgtatggcgctaaaggcatcactaccaaatacacgattacgcaaaaacaaaatcccttaatcaccaacatccgcggcaccaacattgaagaatttctgaccttcggcggaacggatctgaacatcattacatctgcccaaagcaacgacatctataccaatctgttagcagattataagaaaatcgccagcaaattatctaaagttcaggtcagcaatccgctgttaaacccgtataaagatgtgttcgaagcgaaatacggcttggacaaagacgctagtggcatctattccgtcaatattaataaatttaacgatattttcaaaaaattatattccttcaccgaatttgatttgcgcaccaaattccaggtcaaatgtcgtcaaacctatattggccaatacaaatattttaaactgagcaacctgcttaatgattccatctacaatattagtgaaggttacaatattaataacctgaaagttaactttcgtgggcaaaatgcgaatctgaacccccgcatcattacacccatcacgggccgtgggttggtcaaaaaaattattcgcttttgtaagaatatcgtgagcgtgaagggtattcgcaaaagtatctgtatcgaaatcaataatggcgaactgtttttcgtcgcatctgaaaactcgtataacgatgacaatatcaacacaccgaaagaaattgatgacactgtcacttctaacaacaattacgaaaacgacctggaccaggtgatcctcaatttcaatagcgaaagcgcacccggcctgagcgatgaaaaacttaatctcacgattcagaacgacgcctacattccaaaatacgatagtaatggtacatctgatattgaacagcatgatgtcaacgaattaaatgttttcttttacctcgatgcccagaaagtgccggaaggtgagaacaacgtaaatctgacctcttcgattgatacggcattattagaacagccgaaaatttatactttcttttcgtccgaatttattaacaatgttaacaaaccggttcaagcggcgttattcgtttcctggattcagcaagttcttgtagattttacaaccgaggctaatcagaagagcacggtggataagatcgccgacatcagcatcgtcgtgccctacattggtttggcattaaacattggtaatgaggcgcaaaaggggaactttaaagacgccctggaattattaggagcaggtattctgctggagttcgaacctgagctgctgattccgactattttagtgttcaccattaaatccttcttaggctctagtgacaacaaaaataaagtgattaaagcgatcaataatgcccttaaagaacgtgatgagaaatggaaagaagtctactccttcattgtctcaaattggatgacgaaaatcaacacgcagtttaataaacgcaaagaacagatgtatcaggcgctgcaaaaccaggttaatgcgatcaagacaattattgaatctaagtacaactcgtacaccctggaggagaaaaatgaactgactaataagtacgatattaaacaaatcgaaaacgaattgaatcagaaagtctccatcgctatgaacaatatcgatcgctttctgaccgaaagctctatttcctatttgatgaaaattatcaatgaagtcaaaatcaacaaacttcgcgaatatgatgagaacgtaaaaacgtacctgctcaattatattattcaacatgggtcgattctgggcgagtctcaacaagaattgaactcgatggtgacggatactttgaataactcgattccgtttaaattatcgtcatacaccgatgataaaattcttatctcgtacttcaacaaattctttaagcggatcaaaagcagcagcgtccttaatatgcgctataaaaacgataagtacgtagatacgtctggatacgacagtaacattaatattaatggggacgtctataaatatccgacaaataaaaaccaattcgggatttataatgataaactttcggaggtgaacatcagccagaacgattatattatttacgataataaatacaaaaacttcagcatttctttttgggtgcgtatcccaaattacgacaacaaaattgtgaacgtgaataacgaatacacgatcattaattgcatgcgcgataacaattctggttggaaagttagcctgaatcacaatgagattatctggacttttgaggacaatcgtggtatcaaccaaaaattagcgttcaactacggtaatgccaacggtatttctgactacatcaataagtggatctttgtgaccatcaccaatgaccgcctcggcgatagcaagctgtacattaacggtaacctgatcgaccagaaatctattctgaacctgggtaacattcacgtaagtgacaacatcctttttaaaattgtcaattgctcgtatactcgttatatcggcattcgctatttcaatattttcgacaaagaactggatgagacggaaatccagactctgtattctaacgaaccgaacaccaacatcctgaaggacttttgggggaattatcttctctacgataaagagtactaccttcttaacgtgttgaagccgaacaacttcattgatcgtcgtaaggatagcaccttgagcattaacaacattcgtagcaccattttactggcaaaccgcctgtacagcggcattaaagtcaaaattcagcgtgtcaataactccagtacgaatgacaatctggtgcggaaaaatgaccaagtctatattaactttgtcgcaagcaaaactcacctctttccattatatgcggatacagctaccaccaataaagaaaaaactattaaaatctcctcttccgggaaccgctttaatcaggtggtagttatgaactcggtcggcaattgtactatgaattttaaaaataataacggcaataacatcggcctgctgggcttcaaagctgatacagttgtggccagcacctggtattacacccacatgcgtgatcataccaatagtaatggctgcttttggaattttatttctgaagagcacggctggcaagaaaaataagtcgacaagcttgcggccgcactcgag

SEQ ID NO: 99 - DNA, 40 bp, Artificial Sequence
  primer_bind (1)...(40): Synthetic DNA oligonucleotide used as sense primer for R177G site-directed mutagenesis
tatatgccgtcgaaccatggctttggctcaatcgcaattg

SEQ ID NO: 100 - DNA, 40 bp, Artificial Sequence
  primer_bind (1)...(40): Synthetic DNA oligonucleotide used as antisense primer for R177G site-directed mutagenesis
caattgcgattgagccaaagccatggttcgacggcatata

SEQ ID NO: 101 - DNA, 34 bp, Artificial Sequence
  primer_bind (1)...(34): Synthetic DNA oligonucleotide used as sense primer for C198S site-directed mutagenesis
cgttttaacgacaacagcatgaatgaatttatcc

SEQ ID NO: 102 - DNA, 34 bp, Artificial Sequence
  primer_bind (1)...(34): Synthetic DNA oligonucleotide used as antisense primer for C198S site-directed mutagenesis
ggataaattcattcatgctgttgtcgttaaaacg

SEQ ID NO: 103 - DNA, 40 bp, Artificial Sequence
  primer_bind (1)...(40): Synthetic DNA oligonucleotide used as sense primer for R340A site-directed mutagenesis
ccttcaccgaatttgatttggccaccaaattccaggtcaa

SEQ ID NO: 104 - DNA, 40 bp, Artificial Sequence
  primer_bind (1)...(40): Synthetic DNA oligonucleotide used as antisense primer for R340A site-directed mutagenesis
ttgacctggaatttggtggccaaatcaaattcggtgaagg

SEQ ID NO: 105 - DNA, 38 bp, Artificial Sequence
  primer_bind (1)...(38): Synthetic DNA oligonucleotide used as sense primer for I773L site-directed mutagenesis
ctatttcctatttgatgaaacttatcaatgaagtcaaa

SEQ ID NO: 106 - DNA, 38 bp, Artificial Sequence
  primer_bind (1)...(38): Synthetic DNA oligonucleotide used as antisense primer for I773L site-directed mutagenesis
tttgacttcattgataagtttcatcaaataggaaatag

SEQ ID NO: 107 - DNA, 45 bp, Artificial Sequence
  primer_bind (1)...(45): Synthetic DNA oligonucleotide used as sense primer for F963L/E964Q/R967A site-directed mutagenesis
tatctggactcttcaggacaatgctggtatcaaccaaaaattagc

SEQ ID NO: 108 - DNA, 45 bp, Artificial Sequence
  primer_bind (1)...(45): Synthetic DNA oligonucleotide used as antisense primer for F963L/E964Q/R967A site-directed mutagenesis
gctaatttttggttgataccagcattgtcctgaagagtccagata

SEQ ID NO: 109 - DNA, 36 bp, Artificial Sequence
  primer_bind (1)...(36): Synthetic DNA oligonucleotide used as sense primer for +N1196 site-directed mutagenesis
gttatgaactcggtcggcaacaattgtactatgaat

SEQ ID NO: 110 - DNA, 36 bp, Artificial Sequence
  primer_bind (1)...(36): Synthetic DNA oligonucleotide used as antisense primer for +N1196 site-directed mutagenesis
attcatagtacaattgttgccgaccgagttcataac

SEQ ID NO: 111 - DNA, 43 bp, Artificial Sequence
  primer_bind (1)...(43): Synthetic DNA oligonucleotide used as sense primer for H216Y site-directed mutagenesis
gctgactttgatgcatgaactgatctatagcttgcacggcctg

SEQ ID NO: 112 - DNA, 43 bp, Artificial Sequence
  primer_bind (1)...(43): Synthetic DNA oligonucleotide used as antisense primer for H216Y site-directed mutagenesis
caggccgtgcaagctatagatcagttcatgcatcaaagtcagc

SEQ ID NO: 113 - DNA, 43 bp, Artificial Sequence
  primer_bind (1)...(43): Synthetic DNA oligonucleotide used as sense primer for E213Q site-directed mutagenesis
gctgactttgatgcatcaactgatccatagcttgcacggcctg

SEQ ID NO: 114 - DNA, 43 bp, Artificial Sequence
  primer_bind (1)...(43): Synthetic DNA oligonucleotide used as antisense primer for E213Q site-directed mutagenesis
caggccgtgcaagctatggatcagttgatgcatcaaagtcagc

SEQ ID NO: 115 - DNA, 43 bp, Artificial Sequence
  primer_bind (1)...(43): Synthetic DNA oligonucleotide used as sense primer for Q213E site-directed mutagenesis
gctgactttgatgcatgaactgatccatagcttgcacggcctg

SEQ ID NO: 116 - DNA, 43 bp, Artificial Sequence
  primer_bind (1)...(43): Synthetic DNA oligonucleotide used as antisense primer for Q213E site-directed mutagenesis
caggccgtgcaagctatggatcagttcatgcatcaaagtcagc

SEQ ID NO: 117 - DNA, 3814 bp, Artificial Sequence
  mat_peptide (1)...(3814): BoNT/E, E. coli-modified active
gatatcggatccgaattcgagctcccatatgcctaaaatcaattcgttcaactataatgacccggttaacgatcgcacgatcctgtatatcaagccaggtggatgtcaagaattttataaatcattcaacatcatgaaaaatatttggattatcccggaacgcaacgtgatcggcacgacgcctcaagattttcacccgccgacctccctgaaaaatggcgacagttcctactatgacccgaattatttacaatcggatgaagaaaaagatcgtttcctcaagatcgtcacgaaaattttcaaccgcatcaataacaatctgtccggtggcatcttacttgaggaattatctaaagctaatccgtatctggggaacgataataccccggataatcagttccacattggcgatgcgagcgctgtggaaattaaattcagcaacggcagtcaagatattcttctcccaaacgtgattatcatgggggctgaacctgatcttttcgaaactaatagttccaatatttcactgcgcaataattatatgccgtcgaaccatggctttggctcaatcgcaattgtgacgttctcacctgaatatagttttcgttttaacgacaacagcatgaatgaatttatccaagacccggcgctgactttgatgcatgaactgatccatagcttgcacggcctgtatggcgctaaaggcatcactaccaaatacacgattacgcaaaaacaaaatcccttaatcaccaacatccgcggcaccaacattgaagaatttctgaccttcggcggaacggatctgaacatcattacatctgcccaaagcaacgacatctataccaatctgttagcagattataagaaaatcgccagcaaattatctaaagttcaggtcagcaatccgctgttaaacccgtataaagatgtgttcgaagcgaaatacggcttggacaaagacgctagtggcatctattccgtcaatattaataaatttaacgatattttcaaaaaattatattccttcaccgaatttgatttggccaccaaattccaggtcaaatgtcgtcaaacctatattggccaatacaaatattttaaactgagcaacctgcttaatgattccatctacaatattagtgaaggttacaatattaataacctgaaagttaactttcgtgggcaaaatgcgaatctgaacccccgcatcattacacccatcacgggccgtgggttggtcaaaaaaattattcgcttttgtaagaatatcgtgagcgtgaagggtattcgcaaaagtatctgtatcgaaatcaataatggcgaactgtttttcgtcgcatctgaaaactcgtataacgatgacaatatcaacacaccgaaagaaattgatgacactgtcacttctaacaacaattacgaaaacgacctggaccaggtgatcctcaatttcaatagcgaaagcgcacccggcctgagcgatgaaaaacttaatctcacgattcagaacgacgcctacattccaaaatacgatagtaatggtacatctgatattgaacagcatgatgtcaacgaattaaatgttttcttttacctcgatgcccagaaagtgccggaaggtgagaacaacgtaaatctgacctcttcgattgatacggcattattagaacagccgaaaatttatactttcttttcgtccgaatttattaacaatgttaacaaaccggttcaagcggcgttattcgtttcctggattcagcaagttcttgtagattttacaaccgaggctaatcagaagagcacggtggataagatcgccgacatcagcatcgtcgtgccctacattggtttggcattaaacattggtaatgaggcgcaaaaggggaactttaaagacgccctggaattattaggagcaggtattctgctggagttcgaacctgagctgctgattccgactattttagtgttcaccattaaatccttcttaggctctagtgacaacaaaaataaagtgattaaagcgatcaataatgcccttaaagaacgtgatgagaaatggaaagaagtctactccttcattgtctcaaattggatgacgaaaatcaacacgcagtttaataaacgcaaagaacagatgtatcaggcgctgcaaaaccaggttaatgcgatcaagacaattattgaatctaagtacaactcgtacaccctggaggagaaaaatgaactgactaataagtacgatattaaacaaatcgaaaacgaattgaatcagaaagtctccatcgctatgaacaatatcgatcgctttctgaccgaaagctctatttcctatttgatgaaacttatcaatgaagtcaaaatcaacaaacttcgcgaatatgatgagaacgtaaaaacgtacctgctcaattatattattcaacatgggtcgattctgggcgagtctcaacaagaattgaactcgatggtgacggatactttgaataactcgattccgtttaaattatcgtcatacaccgatgataaaattcttatctcgtacttcaacaaattctttaagcggatcaaaagcagcagcgtccttaatatgcgctataaaaacgataagtacgtagatacgtctggatacgacagtaacattaatattaatggggacgtctataaatatccgacaaataaaaaccaattcgggatttataatgataaactttcggaggtgaacatcagccagaacgattatattatttacgataataaatacaaaaacttcagcatttctttttgggtgcgtatcccaaattacgacaacaaaattgtgaacgtgaataacgaatacacgatcattaattgcatgcgcgataacaattctggttggaaagttagcctgaatcacaatgagattatctggactcttcaggacaatgctggtatcaaccaaaaattagcgttcaactacggtaatgccaacggtatttctgactacatcaataagtggatctttgtgaccatcaccaatgaccgcctcggcgatagcaagctgtacattaacggtaacctgatcgaccagaaatctattctgaacctgggtaacattcacgtaagtgacaacatcctttttaaaattgtcaattgctcgtatactcgttatatcggcattcgctatttcaatattttcgacaaagaactggatgagacggaaatccagactctgtattctaacgaaccgaacaccaacatcctgaaggacttttgggggaattatcttctctacgataaagagtactaccttcttaacgtgttgaagccgaacaacttcattgatcgtcgtaaggatagcaccttgagcattaacaacattcgtagcaccattttactggcaaaccgcctgtacagcggcattaaagtcaaaattcagcgtgtcaataactccagtacgaatgacaatctggtgcggaaaaatgaccaagtctatattaactttgtcgcaagcaaaactcacctctttccattatatgcggatacagctaccaccaataaagaaaaaactattaaaatctcctcttccgggaaccgctttaatcaggtggtagttatgaactcggtcggcaacaattgtactatgaattttaaaaataataacggcaataacatcggcctgctgggcttcaaagctgatacagttgtggccagcacctggtattacacccacatgcgtgatcataccaatagtaatggctgcttttggaattttatttctgaagagcacggctggcaagaaaaataagtcgacaagcttgcggccgcactcgag

SEQ ID NO: 118 - DNA, 3814 bp, Artificial Sequence
  mat_peptide (1)...(3814): BoNT/E, E.coli-modified, H216
gatatcggatccgaattcgagctcccatatgcctaaaatcaattcgttcaactataatgacccggttaacgatcgcacgatcctgtatatcaagccaggtggatgtcaagaattttataaatcattcaacatcatgaaaaatatttggattatcccggaacgcaacgtgatcggcacgacgcctcaagattttcacccgccgacctccctgaaaaatggcgacagttcctactatgacccgaattatttacaatcggatgaagaaaaagatcgtttcctcaagatcgtcacgaaaattttcaaccgcatcaataacaatctgtccggtggcatcttacttgaggaattatctaaagctaatccgtatctggggaacgataataccccggataatcagttccacattggcgatgcgagcgctgtggaaattaaattcagcaacggcagtcaagatattcttctcccaaacgtgattatcatgggggctgaacctgatcttttcgaaactaatagttccaatatttcactgcgcaataattatatgccgtcgaaccatggctttggctcaatcgcaattgtgacgttctcacctgaatatagttttcgttttaacgacaacagcatgaatgaatttatccaagacccggcgctgactttgatgcatgaactgatctatagcttgcacggcctgtatggcgctaaaggcatcactaccaaatacacgattacgcaaaaacaaaatcccttaatcaccaacatccgcggcaccaacattgaagaatttctgaccttcggcggaacggatctgaacatcattacatctgcccaaagcaacgacatctataccaatctgttagcagattataagaaaatcgccagcaaattatctaaagttcaggtcagcaatccgctgttaaacccgtataaagatgtgttcgaagcgaaatacggcttggacaaagacgctagtggcatctattccgtcaatattaataaatttaacgatattttcaaaaaattatattccttcaccgaatttgatttggccaccaaattccaggtcaaatgtcgtcaaacctatattggccaatacaaatattttaaactgagcaacctgcttaatgattccatctacaatattagtgaaggttacaatattaataacctgaaagttaactttcgtgggcaaaatgcgaatctgaacccccgcatcattacacccatcacgggccgtgggttggtcaaaaaaattattcgcttttgtaagaatatcgtgagcgtgaagggtattcgcaaaagtatctgtatcgaaatcaataatggcgaactgtttttcgtcgcatctgaaaactcgtataacgatgacaatatcaacacaccgaaagaaattgatgacactgtcacttctaacaacaattacgaaaacgacctggaccaggtgatcctcaatttcaatagcgaaagcgcacccggcctgagcgatgaaaaacttaatctcacgattcagaacgacgcctacattccaaaatacgatagtaatggtacatctgatattgaacagcatgatgtcaacgaattaaatgttttcttttacctcgatgcccagaaagtgccggaaggtgagaacaacgtaaatctgacctcttcgattgatacggcattattagaacagccgaaaatttatactttcttttcgtccgaatttattaacaatgttaacaaaccggttcaagcggcgttattcgtttcctggattcagcaagttcttgtagattttacaaccgaggctaatcagaagagcacggtggataagatcgccgacatcagcatcgtcgtgccctacattggtttggcattaaacattggtaatgaggcgcaaaaggggaactttaaagacgccctggaattattaggagcaggtattctgctggagttcgaacctgagctgctgattccgactattttagtgttcaccattaaatccttcttaggctctagtgacaacaaaaataaagtgattaaagcgatcaataatgcccttaaagaacgtgatgagaaatggaaagaagtctactccttcattgtctcaaattggatgacgaaaatcaacacgcagtttaataaacgcaaagaacagatgtatcaggcgctgcaaaaccaggttaatgcgatcaagacaattattgaatctaagtacaactcgtacaccctggaggagaaaaatgaactgactaataagtacgatattaaacaaatcgaaaacgaattgaatcagaaagtctccatcgctatgaacaatatcgatcgctttctgaccgaaagctctatttcctatttgatgaaacttatcaatgaagtcaaaatcaacaaacttcgcgaatatgatgagaacgtaaaaacgtacctgctcaattatattattcaacatgggtcgattctgggcgagtctcaacaagaattgaactcgatggtgacggatactttgaataactcgattccgtttaaattatcgtcatacaccgatgataaaattcttatctcgtacttcaacaaattctttaagcggatcaaaagcagcagcgtccttaatatgcgctataaaaacgataagtacgtagatacgtctggatacgacagtaacattaatattaatggggacgtctataaatatccgacaaataaaaaccaattcgggatttataatgataaactttcggaggtgaacatcagccagaacgattatattatttacgataataaatacaaaaacttcagcatttctttttgggtgcgtatcccaaattacgacaacaaaattgtgaacgtgaataacgaatacacgatcattaattgcatgcgcgataacaattctggttggaaagttagcctgaatcacaatgagattatctggactcttcaggacaatgctggtatcaaccaaaaattagcgttcaactacggtaatgccaacggtatttctgactacatcaataagtggatctttgtgaccatcaccaatgaccgcctcggcgatagcaagctgtacattaacggtaacctgatcgaccagaaatctattctgaacctgggtaacattcacgtaagtgacaacatcctttttaaaattgtcaattgctcgtatactcgttatatcggcattcgctatttcaatattttcgacaaagaactggatgagacggaaatccagactctgtattctaacgaaccgaacaccaacatcctgaaggacttttgggggaattatcttctctacgataaagagtactaccttcttaacgtgttgaagccgaacaacttcattgatcgtcgtaaggatagcaccttgagcattaacaacattcgtagcaccattttactggcaaaccgcctgtacagcggcattaaagtcaaaattcagcgtgtcaataactccagtacgaatgacaatctggtgcggaaaaatgaccaagtctatattaactttgtcgcaagcaaaactcacctctttccattatatgcggatacagctaccaccaataaagaaaaaactattaaaatctcctcttccgggaaccgctttaatcaggtggtagttatgaactcggtcggcaacaattgtactatgaattttaaaaataataacggcaataacatcggcctgctgggcttcaaagctgatacagttgtggccagcacctggtattacacccacatgcgtgatcataccaatagtaatggctgcttttggaattttatttctgaagagcacggctggcaagaaaaataagtcgacaagcttgcggccgcactcgag

SEQ ID NO: 119 - DNA, 3814 bp, Artificial Sequence
  mat_peptide (1)...(3814): BoNT/E E.coli-modified, E213
gatatcggatccgaattcgagctcccatatgcctaaaatcaattcgttcaactataatgacccggttaacgatcgcacgatcctgtatatcaagccaggtggatgtcaagaattttataaatcattcaacatcatgaaaaatatttggattatcccggaacgcaacgtgatcggcacgacgcctcaagattttcacccgccgacctccctgaaaaatggcgacagttcctactatgacccgaattatttacaatcggatgaagaaaaagatcgtttcctcaagatcgtcacgaaaattttcaaccgcatcaataacaatctgtccggtggcatcttacttgaggaattatctaaagctaatccgtatctggggaacgataataccccggataatcagttccacattggcgatgcgagcgctgtggaaattaaattcagcaacggcagtcaagatattcttctcccaaacgtgattatcatgggggctgaacctgatcttttcgaaactaatagttccaatatttcactgcgcaataattatatgccgtcgaaccatggctttggctcaatcgcaattgtgacgttctcacctgaatatagttttcgttttaacgacaacagcatgaatgaatttatccaagacccggcgctgactttgatgcatcaactgatccatagcttgcacggcctgtatggcgctaaaggcatcactaccaaatacacgattacgcaaaaacaaaatcccttaatcaccaacatccgcggcaccaacattgaagaatttctgaccttcggcggaacggatctgaacatcattacatctgcccaaagcaacgacatctataccaatctgttagcagattataagaaaatcgccagcaaattatctaaagttcaggtcagcaatccgctgttaaacccgtataaagatgtgttcgaagcgaaatacggcttggacaaagacgctagtggcatctattccgtcaatattaataaatttaacgatattttcaaaaaattatattccttcaccgaatttgatttggccaccaaattccaggtcaaatgtcgtcaaacctatattggccaatacaaatattttaaactgagcaacctgcttaatgattccatctacaatattagtgaaggttacaatattaataacctgaaagttaactttcgtgggcaaaatgcgaatctgaacccccgcatcattacacccatcacgggccgtgggttggtcaaaaaaattattcgcttttgtaagaatatcgtgagcgtgaagggtattcgcaaaagtatctgtatcgaaatcaataatggcgaactgtttttcgtcgcatctgaaaactcgtataacgatgacaatatcaacacaccgaaagaaattgatgacactgtcacttctaacaacaattacgaaaacgacctggaccaggtgatcctcaatttcaatagcgaaagcgcacccggcctgagcgatgaaaaacttaatctcacgattcagaacgacgcctacattccaaaatacgatagtaatggtacatctgatattgaacagcatgatgtcaacgaattaaatgttttcttttacctcgatgcccagaaagtgccggaaggtgagaacaacgtaaatctgacctcttcgattgatacggcattattagaacagccgaaaatttatactttcttttcgtccgaatttattaacaatgttaacaaaccggttcaagcggcgttattcgtttcctggattcagcaagttcttgtagattttacaaccgaggctaatcagaagagcacggtggataagatcgccgacatcagcatcgtcgtgccctacattggtttggcattaaacattggtaatgaggcgcaaaaggggaactttaaagacgccctggaattattaggagcaggtattctgctggagttcgaacctgagctgctgattccgactattttagtgttcaccattaaatccttcttaggctctagtgacaacaaaaataaagtgattaaagcgatcaataatgcccttaaagaacgtgatgagaaatggaaagaagtctactccttcattgtctcaaattggatgacgaaaatcaacacgcagtttaataaacgcaaagaacagatgtatcaggcgctgcaaaaccaggttaatgcgatcaagacaattattgaatctaagtacaactcgtacaccctggaggagaaaaatgaactgactaataagtacgatattaaacaaatcgaaaacgaattgaatcagaaagtctccatcgctatgaacaatatcgatcgctttctgaccgaaagctctatttcctatttgatgaaacttatcaatgaagtcaaaatcaacaaacttcgcgaatatgatgagaacgtaaaaacgtacctgctcaattatattattcaacatgggtcgattctgggcgagtctcaacaagaattgaactcgatggtgacggatactttgaataactcgattccgtttaaattatcgtcatacaccgatgataaaattcttatctcgtacttcaacaaattctttaagcggatcaaaagcagcagcgtccttaatatgcgctataaaaacgataagtacgtagatacgtctggatacgacagtaacattaatattaatggggacgtctataaatatccgacaaataaaaaccaattcgggatttataatgataaactttcggaggtgaacatcagccagaacgattatattatttacgataataaatacaaaaacttcagcatttctttttgggtgcgtatcccaaattacgacaacaaaattgtgaacgtgaataacgaatacacgatcattaattgcatgcgcgataacaattctggttggaaagttagcctgaatcacaatgagattatctggactcttcaggacaatgctggtatcaaccaaaaattagcgttcaactacggtaatgccaacggtatttctgactacatcaataagtggatctttgtgaccatcaccaatgaccgcctcggcgatagcaagctgtacattaacggtaacctgatcgaccagaaatctattctgaacctgggtaacattcacgtaagtgacaacatcctttttaaaattgtcaattgctcgtatactcgttatatcggcattcgctatttcaatattttcgacaaagaactggatgagacggaaatccagactctgtattctaacgaaccgaacaccaacatcctgaaggacttttgggggaattatcttctctacgataaagagtactaccttcttaacgtgttgaagccgaacaacttcattgatcgtcgtaaggatagcaccttgagcattaacaacattcgtagcaccattttactggcaaaccgcctgtacagcggcattaaagtcaaaattcagcgtgtcaataactccagtacgaatgacaatctggtgcggaaaaatgaccaagtctatattaactttgtcgcaagcaaaactcacctctttccattatatgcggatacagctaccaccaataaagaaaaaactattaaaatctcctcttccgggaaccgctttaatcaggtggtagttatgaactcggtcggcaacaattgtactatgaattttaaaaataataacggcaataacatcggcctgctgggcttcaaagctgatacagttgtggccagcacctggtattacacccacatgcgtgatcataccaatagtaatggctgcttttggaattttatttctgaagagcacggctggcaagaaaaataagtcgacaagcttgcggccgcactcgag

SEQ ID NO: 120 - DNA, 35 bp, Artificial Sequence
  primer_bind (1)...(35): Synthetic DNA oligonucleotide used as sense primer for  CtermHis
ccgccagcttgtcgactttttcttgccagccgtgc

SEQ ID NO: 121 - DNA, 35 bp, Artificial Sequence
  primer_bind (1)...(35): Synthetic DNA oligonucleotide used as antisense primer for CtermHis
gcacggctggcaagaaaaagtcgacaagctggcgg

SEQ ID NO: 122 - DNA, 3819 bp, Artificial Sequence
  mat_peptide (1)...(3816): Active His-BoNT/E
atgggcagcagccatcatcatcatcatcacagcagcggcctggtgccgcgcggcagccatatgcctaaaatcaattcgttcaactataatgacccggttaacgatcgcacgatcctgtatatcaagccaggtggatgtcaagaattttataaatcattcaacatcatgaaaaatatttggattatcccggaacgcaacgtgatcggcacgacgcctcaagattttcacccgccgacctccctgaaaaatggcgacagttcctactatgacccgaattatttacaatcggatgaagaaaaagatcgtttcctcaagatcgtcacgaaaattttcaaccgcatcaataacaatctgtccggtggcatcttacttgaggaattatctaaagctaatccgtatctggggaacgataataccccggataatcagttccacattggcgatgcgagcgctgtggaaattaaattcagcaacggcagtcaagatattcttctcccaaacgtgattatcatgggggctgaacctgatcttttcgaaactaatagttccaatatttcactgcgcaataattatatgccgtcgaaccatggctttggctcaatcgcaattgtgacgttctcacctgaatatagttttcgttttaacgacaacagcatgaatgaatttatccaagacccggcgctgactttgatgcatgaactgatccatagcttgcacggcctgtatggcgctaaaggcatcactaccaaatacacgattacgcaaaaacaaaatcccttaatcaccaacatccgcggcaccaacattgaagaatttctgaccttcggcggaacggatctgaacatcattacatctgcccaaagcaacgacatctataccaatctgttagcagattataagaaaatcgccagcaaattatctaaagttcaggtcagcaatccgctgttaaacccgtataaagatgtgttcgaagcgaaatacggcttggacaaagacgctagtggcatctattccgtcaatattaataaatttaacgatattttcaaaaaattatattccttcaccgaatttgatttggccaccaaattccaggtcaaatgtcgtcaaacctatattggccaatacaaatattttaaactgagcaacctgcttaatgattccatctacaatattagtgaaggttacaatattaataacctgaaagttaactttcgtgggcaaaatgcgaatctgaacccccgcatcattacacccatcacgggccgtgggttggtcaaaaaaattattcgcttttgtaagaatatcgtgagcgtgaagggtattcgcaaaagtatctgtatcgaaatcaataatggcgaactgtttttcgtcgcatctgaaaactcgtataacgatgacaatatcaacacaccgaaagaaattgatgacactgtcacttctaacaacaattacgaaaacgacctggaccaggtgatcctcaatttcaatagcgaaagcgcacccggcctgagcgatgaaaaacttaatctcacgattcagaacgacgcctacattccaaaatacgatagtaatggtacatctgatattgaacagcatgatgtcaacgaattaaatgttttcttttacctcgatgcccagaaagtgccggaaggtgagaacaacgtaaatctgacctcttcgattgatacggcattattagaacagccgaaaatttatactttcttttcgtccgaatttattaacaatgttaacaaaccggttcaagcggcgttattcgtttcctggattcagcaagttcttgtagattttacaaccgaggctaatcagaagagcacggtggataagatcgccgacatcagcatcgtcgtgccctacattggtttggcattaaacattggtaatgaggcgcaaaaggggaactttaaagacgccctggaattattaggagcaggtattctgctggagttcgaacctgagctgctgattccgactattttagtgttcaccattaaatccttcttaggctctagtgacaacaaaaataaagtgattaaagcgatcaataatgcccttaaagaacgtgatgagaaatggaaagaagtctactccttcattgtctcaaattggatgacgaaaatcaacacgcagtttaataaacgcaaagaacagatgtatcaggcgctgcaaaaccaggttaatgcgatcaagacaattattgaatctaagtacaactcgtacaccctggaggagaaaaatgaactgactaataagtacgatattaaacaaatcgaaaacgaattgaatcagaaagtctccatcgctatgaacaatatcgatcgctttctgaccgaaagctctatttcctatttgatgaaacttatcaatgaagtcaaaatcaacaaacttcgcgaatatgatgagaacgtaaaaacgtacctgctcaattatattattcaacatgggtcgattctgggcgagtctcaacaagaattgaactcgatggtgacggatactttgaataactcgattccgtttaaattatcgtcatacaccgatgataaaattcttatctcgtacttcaacaaattctttaagcggatcaaaagcagcagcgtccttaatatgcgctataaaaacgataagtacgtagatacgtctggatacgacagtaacattaatattaatggggacgtctataaatatccgacaaataaaaaccaattcgggatttataatgataaactttcggaggtgaacatcagccagaacgattatattatttacgataataaatacaaaaacttcagcatttctttttgggtgcgtatcccaaattacgacaacaaaattgtgaacgtgaataacgaatacacgatcattaattgcatgcgcgataacaattctggttggaaagttagcctgaatcacaatgagattatctggactcttcaggacaatgctggtatcaaccaaaaattagcgttcaactacggtaatgccaacggtatttctgactacatcaataagtggatctttgtgaccatcaccaatgaccgcctcggcgatagcaagctgtacattaacggtaacctgatcgaccagaaatctattctgaacctgggtaacattcacgtaagtgacaacatcctttttaaaattgtcaattgctcgtatactcgttatatcggcattcgctatttcaatattttcgacaaagaactggatgagacggaaatccagactctgtattctaacgaaccgaacaccaacatcctgaaggacttttgggggaattatcttctctacgataaagagtactaccttcttaacgtgttgaagccgaacaacttcattgatcgtcgtaaggatagcaccttgagcattaacaacattcgtagcaccattttactggcaaaccgcctgtacagcggcattaaagtcaaaattcagcgtgtcaataactccagtacgaatgacaatctggtgcggaaaaatgaccaagtctatattaactttgtcgcaagcaaaactcacctctttccattatatgcggatacagctaccaccaataaagaaaaaactattaaaatctcctcttccgggaaccgctttaatcaggtggtagttatgaactcggtcggcaacaattgtactatgaattttaaaaataataacggcaataacatcggcctgctgggcttcaaagctgatacagttgtggccagcacctggtattacacccacatgcgtgatcataccaatagtaatggctgcttttggaattttatttctgaagagcacggctggcaagaaaaataa

SEQ ID NO: 123 - protein, 1272 aa, Artificial Sequence
  PEPTIDE (1)...(1272): Active His-BoNT/E
MGSSHHHHHHSSGLVPRGSHMPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 124 - DNA, 3804 bp, Artificial Sequence
  mat_peptide (1)...(3801): Active BoNT/E-His
atgcctaaaatcaattcgttcaactataatgacccggttaacgatcgcacgatcctgtatatcaagccaggtggatgtcaagaattttataaatcattcaacatcatgaaaaatatttggattatcccggaacgcaacgtgatcggcacgacgcctcaagattttcacccgccgacctccctgaaaaatggcgacagttcctactatgacccgaattatttacaatcggatgaagaaaaagatcgtttcctcaagatcgtcacgaaaattttcaaccgcatcaataacaatctgtccggtggcatcttacttgaggaattatctaaagctaatccgtatctggggaacgataataccccggataatcagttccacattggcgatgcgagcgctgtggaaattaaattcagcaacggcagtcaagatattcttctcccaaacgtgattatcatgggggctgaacctgatcttttcgaaactaatagttccaatatttcactgcgcaataattatatgccgtcgaaccatggctttggctcaatcgcaattgtgacgttctcacctgaatatagttttcgttttaacgacaacagcatgaatgaatttatccaagacccggcgctgactttgatgcatgaactgatccatagcttgcacggcctgtatggcgctaaaggcatcactaccaaatacacgattacgcaaaaacaaaatcccttaatcaccaacatccgcggcaccaacattgaagaatttctgaccttcggcggaacggatctgaacatcattacatctgcccaaagcaacgacatctataccaatctgttagcagattataagaaaatcgccagcaaattatctaaagttcaggtcagcaatccgctgttaaacccgtataaagatgtgttcgaagcgaaatacggcttggacaaagacgctagtggcatctattccgtcaatattaataaatttaacgatattttcaaaaaattatattccttcaccgaatttgatttggccaccaaattccaggtcaaatgtcgtcaaacctatattggccaatacaaatattttaaactgagcaacctgcttaatgattccatctacaatattagtgaaggttacaatattaataacctgaaagttaactttcgtgggcaaaatgcgaatctgaacccccgcatcattacacccatcacgggccgtgggttggtcaaaaaaattattcgcttttgtaagaatatcgtgagcgtgaagggtattcgcaaaagtatctgtatcgaaatcaataatggcgaactgtttttcgtcgcatctgaaaactcgtataacgatgacaatatcaacacaccgaaagaaattgatgacactgtcacttctaacaacaattacgaaaacgacctggaccaggtgatcctcaatttcaatagcgaaagcgcacccggcctgagcgatgaaaaacttaatctcacgattcagaacgacgcctacattccaaaatacgatagtaatggtacatctgatattgaacagcatgatgtcaacgaattaaatgttttcttttacctcgatgcccagaaagtgccggaaggtgagaacaacgtaaatctgacctcttcgattgatacggcattattagaacagccgaaaatttatactttcttttcgtccgaatttattaacaatgttaacaaaccggttcaagcggcgttattcgtttcctggattcagcaagttcttgtagattttacaaccgaggctaatcagaagagcacggtggataagatcgccgacatcagcatcgtcgtgccctacattggtttggcattaaacattggtaatgaggcgcaaaaggggaactttaaagacgccctggaattattaggagcaggtattctgctggagttcgaacctgagctgctgattccgactattttagtgttcaccattaaatccttcttaggctctagtgacaacaaaaataaagtgattaaagcgatcaataatgcccttaaagaacgtgatgagaaatggaaagaagtctactccttcattgtctcaaattggatgacgaaaatcaacacgcagtttaataaacgcaaagaacagatgtatcaggcgctgcaaaaccaggttaatgcgatcaagacaattattgaatctaagtacaactcgtacaccctggaggagaaaaatgaactgactaataagtacgatattaaacaaatcgaaaacgaattgaatcagaaagtctccatcgctatgaacaatatcgatcgctttctgaccgaaagctctatttcctatttgatgaaacttatcaatgaagtcaaaatcaacaaacttcgcgaatatgatgagaacgtaaaaacgtacctgctcaattatattattcaacatgggtcgattctgggcgagtctcaacaagaattgaactcgatggtgacggatactttgaataactcgattccgtttaaattatcgtcatacaccgatgataaaattcttatctcgtacttcaacaaattctttaagcggatcaaaagcagcagcgtccttaatatgcgctataaaaacgataagtacgtagatacgtctggatacgacagtaacattaatattaatggggacgtctataaatatccgacaaataaaaaccaattcgggatttataatgataaactttcggaggtgaacatcagccagaacgattatattatttacgataataaatacaaaaacttcagcatttctttttgggtgcgtatcccaaattacgacaacaaaattgtgaacgtgaataacgaatacacgatcattaattgcatgcgcgataacaattctggttggaaagttagcctgaatcacaatgagattatctggactcttcaggacaatgctggtatcaaccaaaaattagcgttcaactacggtaatgccaacggtatttctgactacatcaataagtggatctttgtgaccatcaccaatgaccgcctcggcgatagcaagctgtacattaacggtaacctgatcgaccagaaatctattctgaacctgggtaacattcacgtaagtgacaacatcctttttaaaattgtcaattgctcgtatactcgttatatcggcattcgctatttcaatattttcgacaaagaactggatgagacggaaatccagactctgtattctaacgaaccgaacaccaacatcctgaaggacttttgggggaattatcttctctacgataaagagtactaccttcttaacgtgttgaagccgaacaacttcattgatcgtcgtaaggatagcaccttgagcattaacaacattcgtagcaccattttactggcaaaccgcctgtacagcggcattaaagtcaaaattcagcgtgtcaataactccagtacgaatgacaatctggtgcggaaaaatgaccaagtctatattaactttgtcgcaagcaaaactcacctctttccattatatgcggatacagctaccaccaataaagaaaaaactattaaaatctcctcttccgggaaccgctttaatcaggtggtagttatgaactcggtcggcaacaattgtactatgaattttaaaaataataacggcaataacatcggcctgctgggcttcaaagctgatacagttgtggccagcacctggtattacacccacatgcgtgatcataccaatagtaatggctgcttttggaattttatttctgaagagcacggctggcaagaaaaagtcgacaagcttgcggccgcactcgagcaccaccaccaccaccactga

SEQ ID NO: 125 - protein, 1267 aa, Artificial Sequence
  PEPTIDE (1)...(1267): Active BoNT/E-His
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEKVDKLAAALEHHHHHH

SEQ ID NO: 126 - DNA, 9 bp, Artificial Sequence
  Open reading frame
aaatactta

SEQ ID NO: 127 - DNA, 9 bp, Artificial Sequence
  Synonymous codon open reading frame
aagtatctg

SEQ ID NO: 128 - DNA, 9 bp, Artificial Sequence
  Open reading frame
aaatattta

SEQ ID NO: 129 - DNA, 9 bp, Artificial Sequence
  G+C content open reading frame
aagtacctg

SEQ ID NO: 130 - DNA, 9 bp, Artificial Sequence
  Open reading frame
aaaaaaaaa

SEQ ID NO: 131 - DNA, 9 bp, Artificial Sequence
  polyA region open reading frame
aagaagaag

SEQ ID NO: 132 - DNA, 12 bp, Artificial Sequence
  Open reading frame
acaaccaaaatg

SEQ ID NO: 133 - protein, 4 aa, Artificial Sequence
  Open reading frame
TTKM

SEQ ID NO: 134 - DNA, 12 bp, Artificial Sequence
  translational start site open reading frame
acgactaagatg

SEQ ID NO: 135 - DNA, 9 bp, Artificial Sequence
  Open reading frame
ggtaattgc

SEQ ID NO: 136 - DNA, 9 bp, Artificial Sequence
  RNase cleavage site open reading frame
ggcaactgc

SEQ ID NO: 137 - DNA, 9 bp, Artificial Sequence
  Open reading frame
ggcaactgc

SEQ ID NO: 138 - DNA, 9 bp, Artificial Sequence
  Out of frame stop codon open reading frame
ggtaactgc

SEQ ID NO: 139 - DNA, 12 bp, Artificial Sequence
  Open reading frame
gcttggccaagc

SEQ ID NO: 140 - DNA, 12 bp, Artificial Sequence
  hairpin-loop structure open reading frame
gcatggcctagc